(19) United States
(12) Patent Application Publication
(10) Pub. No.: US 2006/0276406 A1
(43) Pub. Date: Dec. 7, 2006

(54) Methods of treating hepatitis C virus

(75) Inventors: Samir K. Gupta, East Brunswick, NJ (US); Bruce A. Malcolm, Paoli, PA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/443,959

(22) Filed: May 31, 2006

Related U.S. Application Data
(60) Provisional application No. 60/686,946, filed on Jun. 2, 2005.

(51) Int. Cl. A61K 38/05; A61K 38/04; A61K 31/4709

(52) U.S. Cl. 514/18; 514/19; 514/310

(57) ABSTRACT

Methods for preventing, ameliorating or treating one or more symptoms of Hepatitis C virus (HCV), modulating HCV protease activity and/or inhibiting cathepsin activity in a subject, wherein the methods comprise administering to a subject in need of such treatment a dosage formulation containing at least one compound of Formulae I-XXVI herein, wherein the dosage formulation is capable of maintaining an average Cmin plasma concentration of the compound at or above 10 ng/ml.

### CROSS-REFERENCE TO PRIORITY APPLICATION

This application claims the benefit of priority from U.S. provisional patent application Ser. No. 60/686,946 filed Jun. 2, 2005.

### FIELD OF THE INVENTION

The present invention relates to methods for maintaining an average minimum plasma concentration of compounds that are useful for treating a wide variety of diseases or disorders associated with hepatitis C virus by inhibiting HCV protease (for example HCV NS3/NS4a serine protease), and/or diseases or disorders associated with cathepsin activity and inhibiting cathepsin activity.

### BACKGROUND OF THE INVENTION

HCV has been implicated in cirrhosis of the liver and in induction of hepatocellular carcinoma. The prognosis for patients suffering from HCV infection is currently poor. HCV infection is more difficult to treat than other forms of hepatitis due to the lack of immunity or remission associated with HCV infection. Current data indicates a less than 50% survival rate at four years post cirrhosis diagnosis. Patients diagnosed with localized resectable hepatocellular carcinoma have a five-year survival rate of 10-30%, whereas those with localized unresectable hepatocellular carcinoma have a five-year survival rate of less than 1%.

Current therapies for hepatitis C include interferon-α (INF_{α}) and combination therapy with ribavirin and interferon. See, e.g., Beremguer et al. (1998) Proc. Assoc. Am. Physicians 110(2):98-112. These therapies suffer from a low sustained response rate and frequent side effects. See, e.g., Hoofnagle et al. (1 997) N. Engl. J. Med. 336:347. Currently, no vaccine is available for HCV infection.

Hepatitis C virus (HCV) is a (+)-sense single-stranded RNA virus that has been implicated as the major causative agent in non-A, non-B hepatitis (NANBH), particularly in blood-associated NANBH (BB-NANBH)(see, International Patent Application Publication No. WO 89/04669 and European Patent Application Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

Recently, an HCV protease necessary for polypeptide processing and viral replication has been identified, cloned and expressed; (see, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3, 4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. Other chymotrypsin-like enzymes are elastase, factor Xa, thrombin, trypsin, plasmin, urokinase, tPA and PSA. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating four viral proteins during viral replication. This has made the HCV NS3 serine protease an attractive target for antiviral chemotherapy.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e., trans).

Analysis of the natural cleavage sites for HCV protease revealed the presence of cysteine at P1 and serine at P1′ and that these residues are strictly conserved in the NS4a/NS4b, NS4b/NS5a and NS5a/NS5b junctions. The NS3/NS4a junction contains a threonine at P1 and a serine at P1′. The Cys→Thr substitution at NS3/NS4a is postulated to account for the requirement of cis rather than trans processing at this junction. See, eg., Pizzi et al. (1994) Proc. Natl. Acad. Sci (USA) 91:888-892, Failla et al. (1996) Folding & Design 1:35-42. The NS3/NS4a cleavage site is also more tolerant of mutagenesis than the other sites. See, e.g., Kollykhalov et al. (1994) J. Virol. 68:7525-7533. It has also been found that acidic residues in the region upstream of the cleavage site are required for efficient cleavage. See, e. Komoda et al. (1994) J. Virol. 68:7351-7357.

Inhibitors of HCV protease that have been reported include antioxidants (see, International Patent Application Publication No. WO 98/14181), certain peptides and peptide analogs (see, International Patent Application Publication No. WO 98/17679, Landro et al. (1997) Biochem. 36:9340-9348, Ingallinella et al. (1998) Biochem. 37:8906-8914, Llinàs-Brunet et al. (1998) Bioorg. Med. Chem. Lett. 8:1713-1718), inhibitors based on the 70-amino acid polypeptide eglin c (Martin et al. (1998) Biochem. 37:11459-11468, inhibitors affinity selected from human pancreatic secretory trypsin inhibitor (hPSTI-C3) and minibody repertoires (MBip) (Dimasi et al. (1997) J. Virol. 71:7461-7469), cV_{H}E2 (a “camelized” variable domain antibody fragment) (Martin et al. (1997) Protein Eng. 10:607-614), and α1-antichymotrypsin (ACT) (Elzouki et al.) (1997) J. Hepat. 27:42-28). A ribozyme designed to selectively destroy hepatitis C virus RNA has recently been disclosed (see, BioWorld Today 9(217): 4 (Nov. 10, 1998)).

Reference is also made to the PCT Publications, No. WO 98/17679, published Apr. 30, 1998 (Vertex Pharmaceuticals Incorporated); WO 98/22496, published May 28, 1998 (F. Hoffmann-La Roche AG); and WO 99/07734, published Feb. 18, 1999 (Boehringer Ingelheim Canada Ltd.).

Pending and copending U. S. patent applications, Ser. No. 60/194,607, filed Apr. 5, 2000, and Ser. No. 60/198,204, filed Apr. 19, 2000, Ser. No. 60/220,110, filed Jul. 21, 2000, Ser. No. 60/220,109, filed Jul. 21, 2000, Ser. No. 60/220,107, filed Jul. 21, 2000, Ser. No. 60/254,869, filed Dec. 12, 2000, Ser. No. 60/220,101, filed Jul. 21, 2000, Ser. No. 60/568,721 filed May 6, 2004, and WO 2003/062265, disclose various types of peptides and/or other compounds as NS-3 serine protease inhibitors of hepatitis C virus.

There is a need for new treatments and therapies for HCV infection to provide methods of treatment useful in the treatment or prevention or amelioration of one or more symptoms of hepatitis C, methods for modulating the activity of serine proteases, particularly the HCV NS3/NS4a serine protease, and methods of modulating the processing of the HCV polypeptide using the compounds provided herein.

Another aspect of the present invention is directed to inhibiting cathepsin activity. Cathepsins (Cats) belong to the papain superfamily of lysosomal cysteine proteases. Cathepsins are involved in the normal proteolysis and turnover of target proteins and tissues as well as in initiating proteolytic cascades by proenzyme activation and in participating in MHC class II molecule expression. Baldwin (1993) Proc. Natl. Acad. Sci., 90: 6796-6800; Mixuochi (1994) Immunol. Lett., 43:189-193.

However, aberrant cathepsin expression has also been implicated in several serious human disease states. Cathepsins have been shown to be abundantly expressed in cancer cells, including breast, lung, prostate, glioblastoma and head/neck cancer cells, (Kos et al. (1998) Oncol. Rep., 5:1349-1361; Yan et al. (1998) Biol. Chem., 379:113-123; Mort et al. (1997) Int. J Biochem. Cell Biol., 29: 715-720; Friedrick et al. (1999) Eur. J Cancer, 35:138-144) and are associated with poor treatment outcome of patients with breast cancer, lung cancer, brain tumor and head/neck cancer. Kos et al, supra. Additionally, aberrant expression of cathepsin is evident in several inflammatory disease states, including rheumatoid arthritis and osteoarthritis. Keyszer (1995) Arthritis Rheum., 38:976-984.

The molecular mechanisms of cathepsin activity are not completely understood. Recently, it was shown that forced expression of cathepsin B rescued cells from serum deprivation-induced apoptotic death (Shibata et al. (1998) Biochem. Biophys. Res. Commun., 251: 199-203) and that treatment of cells with antisense oligonucleotides of cathepsin B induced apoptosis. Isahara et at. (1999) Neuroscience, 91:233-249. These reports suggest an anti-apoptotic role for the cathepsins that is contrary to earlier reports that cathepsins are mediators of apoptosis. Roberts et al (1997) Gastroenterology, 113: 1714-1726; Jones et al. (1998) Am. J Physiol., 275: G723-730.

Cathepsin K is a member of the family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J Biol. Chem. 271, 12517-12524; Drake, F. H., et al., (1996) J. Biol. Chem. 271, 12511-12516; Bromme, D., et al., (1996) J. Biol. Chem. 271, 2126-2132.

Cathepsin K has been variously denoted as cathepsin O, cathepsin X or cathepsin O2 in the literature. The designation cathepsin K is considered to be the more appropriate one (name assigned by Nomenclature Committee of the International Union of Biochemistry and Molecular Biology).

Cathepsins of the papain superfamily of cysteine proteases function in the normal physiological process of protein degradation in animals, including humans, e.g., in the degradation of connective tissue. However, elevated levels of these enzymes in the body can result in pathological conditions leading to disease. Thus, cathepsins have been implicated in various disease states, including but not limited to, infections by pneumocystis carinii, trypsanoma cruzi, trypsanoma brucei brucei, and Crithidia fusiculata; as well as in schistosomiasis malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Cathepsin K is believed to play a causative role in diseases of excessive bone or cartilage loss. Bone is composed of a protein matrix in which spindle- or plate-shaped crystals of hydroxyapatite are incorporated. Type I Collagen represents the major structural protein of bone comprising approximately 90% of the structural protein. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodeling at discrete foci throughout life. These foci, or remodeling units, undergo a cycle consisting of a bone resorption phase followed by a phase of bone replacement. Bone resorption is carried out by osteoclasts, which are multinuclear cells of hematopoietic lineage. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

There are reports in the literature of the expression of Cathepsin B and L antigen and that activity is associated with early colorectal cancer progression. Troy et al., (2004) Eur J Cancer, 40(10):1610-6. The findings suggest that cysteine proteases play an important role in colorectal cancer progression.

Cathepsin L has been shown to be an important protein mediating the malignancy of gliomas and it has been suggested that its inhibition may diminish their invasion and lead to increased tumor cell apoptosis by reducing apoptotic threshold. Levicar et al., (2003) Cancer Gene Ther., 10(2): 141-51.

Katunama et al., (2002) Arch Biochem Biophys., 397(2):305-11 reports on antihypercalcemic and antimetastatic effects of CLIK-148 in vivo, which is a specific inhibitor of cathepsin L. This reference also reports that CLIK-148 treatment reduced distant bone metastasis to the femur and tibia of melanoma A375 tumors implanted into the left ventricle of the heart.

Rousselet et al., (2004) Cancer Res., 64(1): 146-51 reports that anti-cathepsin L single chain variable fragment (ScFv) could be used to inhibit the tumorigenic and metastatic phenotype of human melanoma, depending on procathepsin L secretion, and the possible use of anti-cathepsin L ScFv as a molecular tool in a therapeutic cellular approach.

Colella et al., (2003) Biotech Histochem., 78(2):101-8 reports that the cysteine proteinases cathepsin L and B participate in the invasive ability of the PC3 prostrate cancer cell line, and the potential of using cystein protease inhibitors such as cystatins as anti-metastatic agents.

Krueger et al., (2001) Cancer Gene Ther., 8(7):522-8 reports that in human osteosarcoma cell line MNNG/HOS, cathepsin L influences cellular malignancy by promoting migration and basement membrane degradation.

Frohlich et al., (2204) Arch Dermatol Res., 295(10):411-21 reports that cathepsins B and L are involved in invasion of basal cell carcinoma (BCC) cells.

U.S. Provisional Patent Application Ser. No. Not Yet Assigned, entitled “Compounds for Inhibiting Cathepsin Activity”, filed Apr. 20, 2005, discloses various types of peptides and/or other compounds as inhibitors of cathepsin.

Cathepsins therefore are attractive targets for the discovery of novel chemotherapeutics and methods of treatment effective against a variety of diseases. There is a need for treatments useful in the inhibition of cathepsin activity and in the treatment of these disorders.

### SUMMARY OF THE INVENTION

The present invention provides a method for preventing, ameliorating or treating one or more symptoms of Hepatitis C virus (HCV) in a subject, wherein the method comprises administering to a subject in need of such treatment a dosage formulation containing at least one HCV protease inhibitor of various structural formulae set forth below in a pharmaceutically effective amount thereof, wherein the dosage formulation is capable of maintaining an average Cmin plasma concentration of the at least one HCV protease inhibitor at or above 10 ng/ml.

The present invention also provides a method for modulating the activity of Hepatitis C virus (HCV) protease in a subject, wherein the method comprises administering to a subject in need of such treatment a dosage formulation containing at least one HCV protease inhibitor in a pharmaceutically effective amount thereof, wherein the dosage form is capable of maintaining an average Cmin plasma concentration of the at least one HCV protease inhibitor at or above 10 ng/ml, comprising administering to said subject an effective amount of at least one compound of various structural formulae set forth below.

The present invention also provides a method for treating diseases or disorders associated with cathepsin activity and/or for inhibiting cathepsin activity in a subject, wherein the method comprises administering to a subject in need of such treatment at least one HCV protease inhibitor in a pharmaceutically effective amount thereof, wherein the dosage formulation is capable of maintaining an average Cmin plasma concentration of the at least one HCV protease inhibitor at or above 10 ng/ml.

In one embodiment, the HCV protease inhibitor is a compound of structural Formula I 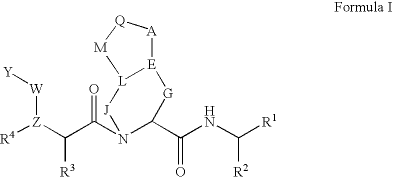
or a pharmaceutically acceptable salt, solvate or ester thereof;
wherein:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²; R¹ is COR⁵ or B(OR)₂, wherein R⁵ is H, OH, OR⁸, NR⁹R¹⁰, CF₃, C₂F₅, C₃F₇, CF₂R⁶, R⁶, or COR⁷ wherein R⁷ is H, OH, OR⁸, CHR⁹R¹⁰, or NR⁹R¹⁰, wherein R⁶, R⁸, R⁹ and R¹⁰ are independently selected from the group consisting of H, alkyl, aryl, heteroalkyl, heteroaryl, cycloalkyl, cycloalkyl, arylalkyl, heteroarylalkyl, [CH(R^{1′})]ₚCOOR¹¹, [CH(R^{1′})]ₚCONR¹²R¹³, [CH(R^{1′})]ₚSO₂R¹¹, [CH(R^{1′})]ₚCOR¹¹, [CH(R^{1′})]ₚCH(OH)RCH(R^{1′})CONHCH(R^{2′})COOR¹¹, CH(R^{1′})CONHCH(R^{2′})CONR¹²R¹³, CH(R^{1′})CONHCH(R^{2′})R′, CH(R^{1′})CONHCH(R^{2′})CONHCH(R^{3′})COOR¹¹, CH(R^{1′})CONHCH(R^{2′})CONHCH(R^{3′}) CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹ and CH(R^{1′})CONHCH(R^{2′})CONHCH(R^{3′})CONHCH(R^{4′})CONHCH(R^{5′})CONR¹²R¹³, wherein R^{1′}, R^{2′}, R^{3′}, R^{4′}, R^{5′}, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;
Z is selected from O, N, CH or CR;
W maybe present or absent, and if W is present, W is selected from C═O, C═S, C(═N—CN), or SO₂;
Q maybe present or absent, and when Q is present, Q is CH, N, P, (CH₂)ₚ, (CHR)ₚ, (CRR′)ₚ, O, NR, S, or SO₂; and when Q is absent, M may be present or absent;
when Q and M are absent, A is directly linked to L;
A is O, CH₂, (CHR)ₚ, (CHR—CHR′)ₚ, (CRR′)ₚ, NR, S, SO₂ or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;
L may be present or absent, and when L is present, L is CH, CR, O, S or NR; and when L is absent, then M may be present or absent; and if M is present with L being absent, then M is directly and independently linked to E, and J is directly and independently linked to E;
M may be present or absent, and when M is present, M is O, NR, S, SO₂, (CH₂)ₚ, (CHR)ₚ(CHR—CHR′)ₚ, or (CRR′)ₚ;
p is a number from 0 to 6; and
R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen; (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic_ring.

In another embodiment, the HCV protease inhibitor is a compound of Formula 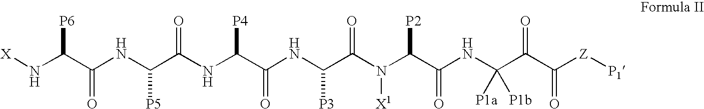
or a pharmaceutically acceptable salt, solvate or ester thereof; wherein:
Z is O, NH or NR¹²;
X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylal kylcarbonyl, arylcarbonyl, heteroarylcarbonyl, alkoxycarbonyl, heterocyclyloxycarbonyl, aryloxycarbonyl, heteroaryloxycarbonyl, alkyaminocarbonyl, heterocyclylaminocarbonyl, arylaminocarbonyl, or heteroarylaminocarbonyl moiety, with the proviso that X may be additionally optionally substituted with R¹² or R¹³;
X¹ is H; C₁-C₄ straight chain alkyl; C₁-C₄ branched alkyl or ; CH₂-aryl (substituted or unsubstituted);
R is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R may be additionally optionally substituted with R13.
R¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³.
P1a, P1b, P2, P3, P4, P5, and P6 are independently:

H; C1-C10 straight or branched chain alkyl; C2-C10 straight or branched chain alkenyl;

C3-C8 cycloalkyl, C3-C8 heterocyclic; (cycloalkyl)alkyl or (heterocyclyl)alkyl, wherein said cycloalkyl is made up of 3 to 8 carbon atoms, and zero to 6 oxygen, nitrogen, sulfur, or phosphorus atoms, and said alkyl is of 1 to 6 carbon atoms;

aryl, heteroaryl, arylalkyl, or heteroarylalkyl, wherein said alkyl is of 1 to 6 carbon atoms;

wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and
P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R13.

In another embodiment, the HCV protease inhibitor is a compound of Formula III 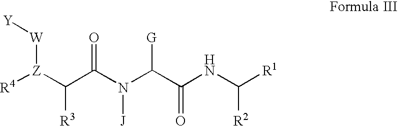
or a pharmaceutically acceptable salt, solvate or ester thereof; wherein:
G, J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe additionally optionally substituted with X¹¹ or X¹²;

X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl, CH(R^{1′})COOR¹¹, CH(R^{1′})CONR¹²R¹³, CH(R^{1′})CONHCH(R^{2′})COOR¹¹, CH(R^{1′})CONHCH(R^{2′})CONR¹²R¹³, CH(R^{1′})CONHCH(R^{2′})R′, CH(R^{1′})CONHCH(R^{2′})CONHCH(R^{3″})COOR¹¹, CH(R^{1′})CONHCH(R^{2′})CONHCH(R^{3′})CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹, and CH(R^{1′})CONHCH(R^{2′})CONHCH(R^{3′})CONHCH(R^{4′})CONHCH(R^{5′}) CONR¹²R¹³, wherein R^{1′}, R^{2′}, R^{3′}, R^{4′}, R^{5′}, R¹¹, R¹², R¹³, and R′ may be the same or different and are independently selected from a group consisting of H, alkyl, aryl, heteroalkyl, heteroaryl, cycloalkyl, alkyl-aryl, alkyl-heteroaryl, aryl-alkyl and heteroaralkyl;
is selected from O, N, or CH;
W maybe present or absent, and if W is present, W is selected from C═O, C═S, or SO₂; and
R, R′, R², R³ and R⁴ are independently selected from the group consisting of H; C1-C10 alkyl; C2-C10 alkenyl; C3-C8 cycloalkyl; C3-C8 heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate.

In another embodiment, the HCV protease inhibitor is a compound of Formula IV 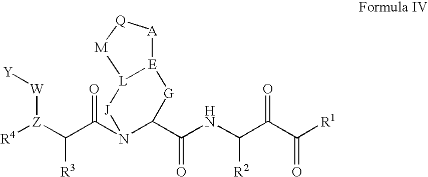
or a pharmaceutically acceptable salt, solvate or ester thereof; wherein:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ s alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is selected from the following structures:

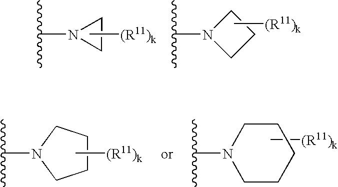

wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹≠H) maybe optionally substituted with X¹¹ or X¹²;
Z is selected from O, N, CH or CR;
W may be present or absent, and if W is present, W is selected from C═O, C═S, C(═N—CN), or S(O₂);
Q may be present or absent, and when Q is present, Q is CH, N, P, (CH₂)ₚ, (CHR)ₚ, (CRR′)ₚ, O, N(R), S, or S(O₂); and when Q is absent, M may be present or absent;
when Q and M are absent, A is directly linked to L;
A is O, CH₂, (CHR)ₚ, (CHR—CHR′)ₚ,(CRR′)ₚ, N(R), S, S(O₂) or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J may be present or absent, and when J is present, J is (CH₂)ₚ, (CHR)ₚ, or (CRR′)ₚ, S(O₂), NH, N(R) or O; and when J is absent, G is present and E is directly linked to N shown in Formula I as linked to J;
L may be present or absent, and when L is present, L is CH, C(R), O, S or N(R); and when L is absent, then M may be present or absent; and if M is present with L being absent, then M is directly and independently linked to E, and J is directly and independently linked to E;
M may be present or absent, and when M is present, M is O, N(R), S, S(O₂), (CH₂)ₚ, (CHR)ₚ(CHR—CHR′)ₚ, or (CRR′)ₚ;
p is a number from 0 to 6; and
R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered cyclic ring structure or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring.

In another embodiment, the HCV protease inhibitor is a compound of Formula V 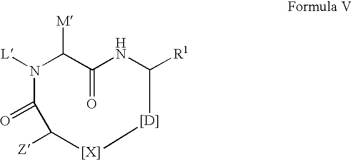
or a pharmaceutically acceptable salt, solvate or ester of said compound wherein:
(1) R¹ is —C(O)R⁵ or —B(OR)₂;
(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;
(3) R⁷ is H, —OH, —OR⁸, or —CHR⁹R¹⁰;
(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R^{1′})CH(R^{1′})C(O)OR¹¹, [CH(R^{1′})]ₚC(O)OR¹¹, —[CH(R^{1′})]ₚC(O)NR¹²R¹³, —[CH(R^{1′})]ₚS(O₂)R¹¹, —[CH(R^{1′})]ₚC(O)R¹¹, —[CH(R^{1′})]ₚS(O₂)NR¹²R¹³, CH(R^{1′})C(O)N(H)CH(R^{2′})(R′), CH(R^{1′})CH(R^{1′})C(O)NR¹²R¹³, —CH(R^{1′})CH(R^{1′})S(O₂)R¹¹, —CH(R^{1′})CH(R^{1′})S(O₂)NR¹²R¹³, —CH(R^{1′})CH(R^{1′})C(O)R¹¹, —[CH(R^{1′})]ₚCH(OH)R¹¹, —CH(R^{1′})C(O)N(H)CH(R^{2′})C(O)OR¹¹, C(O)N(H)CH(R^{2′})C(O)OR¹¹, —C(O)N(H)CH(R^{2′})C(O)R¹¹, CH(R^{1′})C(O)N(H)CH(R^{2′})C(O)NR¹²R¹³, —CH(R^{1′})C(O)N(H)CH(R^{2′})R′, CH(R^{1′})C(O)N(H)CH(R^{2′})C(O)N(H)CH(R^{3′})C(O)OR¹¹, CH(R^{1′})C(O)N(H)CH(R^{2′})C(O)CH(R^{3′})NR¹²R¹³, CH(R^{1′})C(O)N(H)CH(R^{2′})C(O)N(H)CH(R^{3′})C(O)NR¹²R¹³, CH(R^{1′})C(O)N(H)CH(R^{2′})C(O)N(H)CH(R^{3′})C(O)N(H)CH (R^{4′})C(O)OR¹¹, H(R^{1′})C(O)N(H)CH(R^{2′})C(O)N(H)CH(R^{3′})C(O)N(H)CH(R^{4′})C(O)NR¹²R¹³, CH(R^{1′})C(O)N(H)CH(R^{2′})C(O)N(H)CH(R^{3′})C(O)N(H)CH(R^{4′})C(O)N(H)CH(R^{5′})C(O)OR¹¹, and CH(R^{1′})C(O)N(H)CH(R^{2′})C(O)N(H)CH(R^{3′})C(O)N(H)CH(R^{4′})C(O)N(H)CH(R^{5′})C(O)NR¹²R¹³;
wherein R^{1′}, R^{2′}, R^{3′}, R^{4′}, R^{5′}, R¹¹, R¹² and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl; or
R¹² and R¹³ are linked together wherein the combination is cycloalkyl, heterocycloalkyl, ary or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain; or L′ and M′ are linked together to form a ring structure wherein the portion of structural Formula 1 represented by 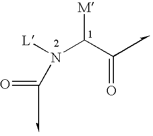
is represented by structural Formula 2: 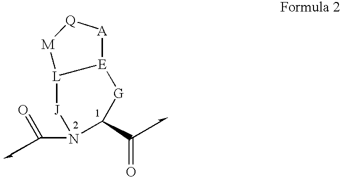
wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;

L is present or absent, and when L is present, L is C(H) or C(R); when L is absent, M is present or absent; if M is present with L being absent, then M is directly and independently linked to E, and J is directly and independently linked to E;

G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;

Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent bing selected from —OR, —CH(R)(R′), S(O)₀₋₂R or —NRR′ or (iii) absent; when both Q and M are absent, A is either directly linked to L, or A is an independent substituent on E, said independent substituent bing selected from —OR,
—CH(R)(R′), S(O)₀₋₂R or —NRR′ or A is absent;
A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, O, OR, N(R), S, S(O₂), (CH₂)ₚ, (CHR)ₚ(CHR—CHR′)ₚ, or (CRR′)ₚ; or M is linked to A to form an alicyclic, aliphatic or heteroalicyclic bridge;
(8) Z′ is represented by the structural Formula 3: 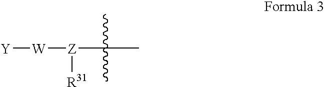
wherein in Formula 3, Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹¹ is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;

Z is O, N, C(H) or C(R);
R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstiuted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;

W may be present or absent, and if W is present, W is C(═O), C(═S), C(═N—CN), or S(O₂);
(9) X is represented by structural Formula 4: 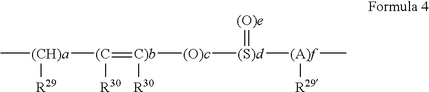
wherein in Formula 4, a is 2, 3, 4, 5, 6, 7, 8 or 9;
b, c, d, e and f are 0, 1, 2, 3, 4 or 5;

A is C, N, S or O;
R²⁹ and R^{29′}are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R²⁹ and R^{29′}are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;
(10) D is represented by structural Formula 5: 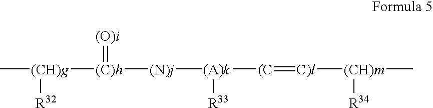
wherein in Formula 5, R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R³² and R³⁴ are linked together such that the combination forms a portion of a cycloalkyl group;

g is 1, 2, 3, 4, 5, 6, 7, 8 or 9;

h, i, j, k, l and m are 0, 1, 2, 3, 4 or 5; and

A is C, N, S or O,
(11) provided that when structural Formula 2: 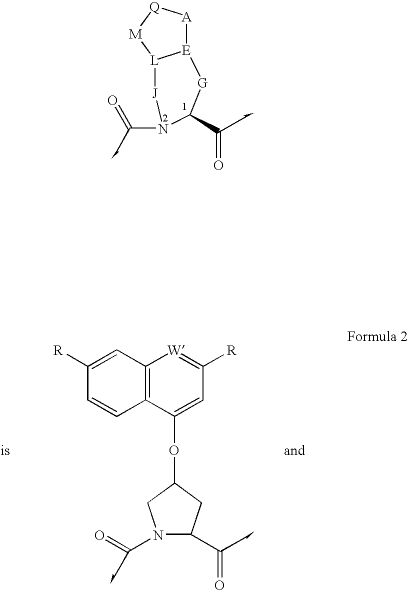
W′ is CH or N, both the following conditional exclusions (i) and (ii) apply:
conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆, alkyl or C₃₋₆ cycloalkyl; and
conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈ alkyl, C₃₋₆ cycloalkyl, C_{6 to 10} aryl or C₇₋₁₆ aralkyl.

In another embodiment, the HCV protease inhibitor is a compound of Formula VI 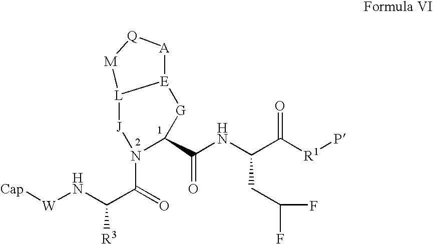
or a pharmaceutically acceptable salt, solvate or ester of said compound, wherein:
Cap and P′ are independently H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino can be unsubstituted or optionally independently substituted with one or two substituents which can be the same or different and are independently selected from X¹ and X²;
X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;
X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;
W may be present or absent, and when W is present W is C(═O), C(═S), C(═NH), C(═N—OH), C(═N—CN), S(O) or S(O₂);

Q maybe present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R′), S(O)₀₋₂R or —NRR′; when both Q and M are absent, A is either directly linked to L, or A is an independent substituent on E, selected from —OR, CH(R)(R′), —S(O)₀₋₂R or —NRR′;

A is present or absent and if present A is —O—, —O(R)CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either Q and E are connected by a bond or Q is an independent substituent on M;

E is present or absent and if present E is CH, N, C(R);

G may be present or absent, and when G is present, G is (CH₂)ₚ, (CHR)ₚ, or (CRR′)ₚ; when G is absent, J is present and E is directly connected to the carbon atom marked position 1;

J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;

L may be present or absent, and when L is present, L is CH, N, or CR; when L is absent, M is present or absent; if M is present with L being absent, then M is directly and independently linked to E, and J is directly and independently linked to E;
M may be present or absent, and when M is present, M is O, N(R), S, S(O₂), (CH₂)ₚ, (CHR)ₚ, (CHR—CHR′)ₚ, or (CRR′)ₚ;

p is a number from 0 to 6;
R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;
R and R′ in (CRR′) can be linked together such that the combination forms a cycloalkyl or heterocyclyl moiety; and

R¹ is N(R) or O.

In another embodiment, the HCV protease inhibitor is a compound of Formula VII 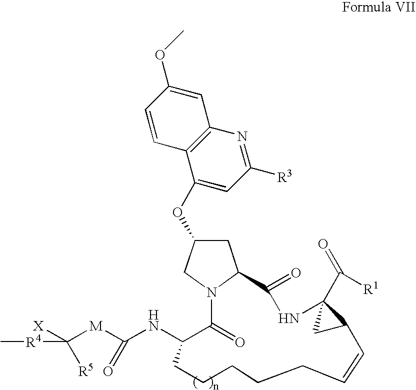
or a pharmaceutically acceptable salt, solvate or ester thereof, wherein,

M is O, N(H), or CH₂;

n is 0-4;

R¹ is —OR⁶, —NR⁶R⁷ or 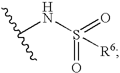
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 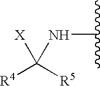
is represented by 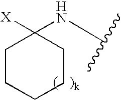
where k is 0 to 2;
X is selected from the group consisting of: 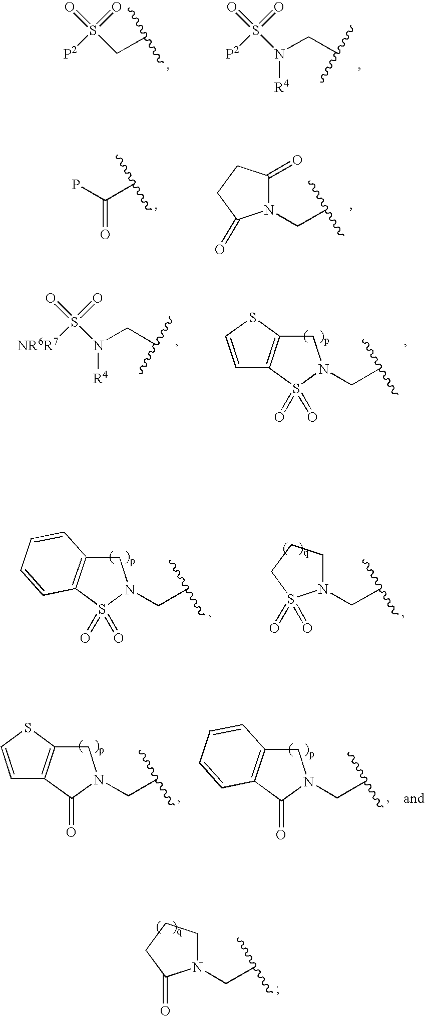
where p is 1 to 2, q is 1-3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 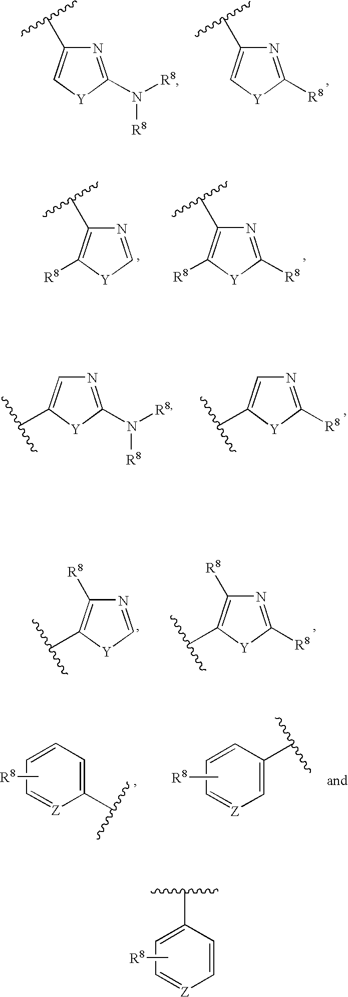
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the HCV protease inhibitor is a compound of Formula VIII: 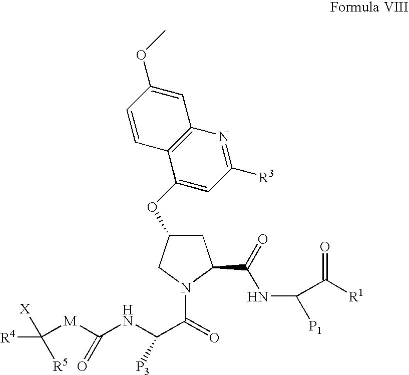
or a pharmaceutically acceptable salt, solvate or ester thereof, wherein,
M is O, N(H), or CH₂;
R¹ is —OR⁶, —NR⁶R⁷ or 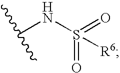
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
P₁ is selected from the group consisting of alkyl, alkenyl, alkynyl, cycloalkyl haloalkyl;
P₃ is selected from the group consisting of alkyl, cycloalkyl, aryl and cycloalkyl fused with aryl;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 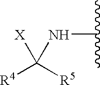
is represented by 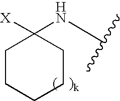
where k is 0 to 2;
X is selected from the group consisting of:

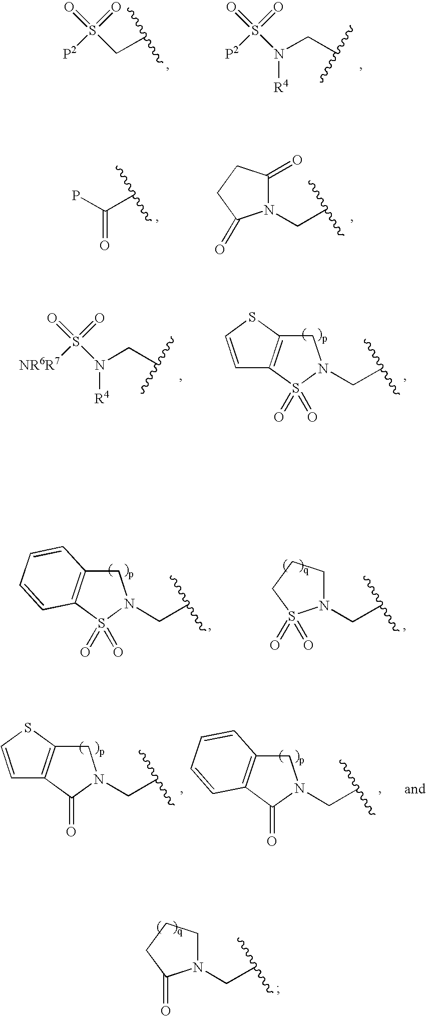

where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 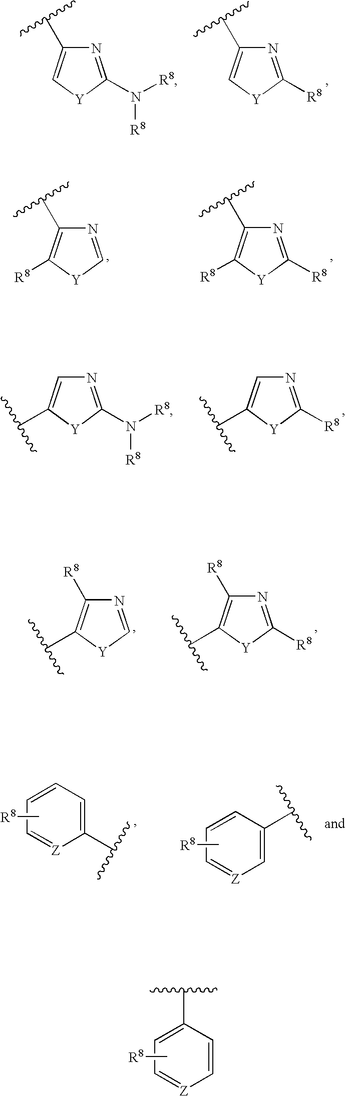
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the HCV protease inhibitor is a compound of Formula IX: 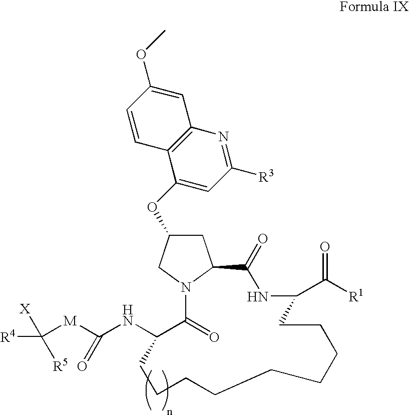
or a pharmaceutically acceptable salt, solvate or ester thereof, wherein,
M is O, N(H), or CH₂;
n is 0-4;
R¹ is —OR⁶, —NR⁶R⁷ or

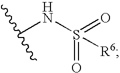

where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 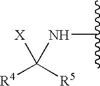
is represented by 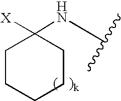
where k is 0 to 2;
X is selected from the group consisting of:

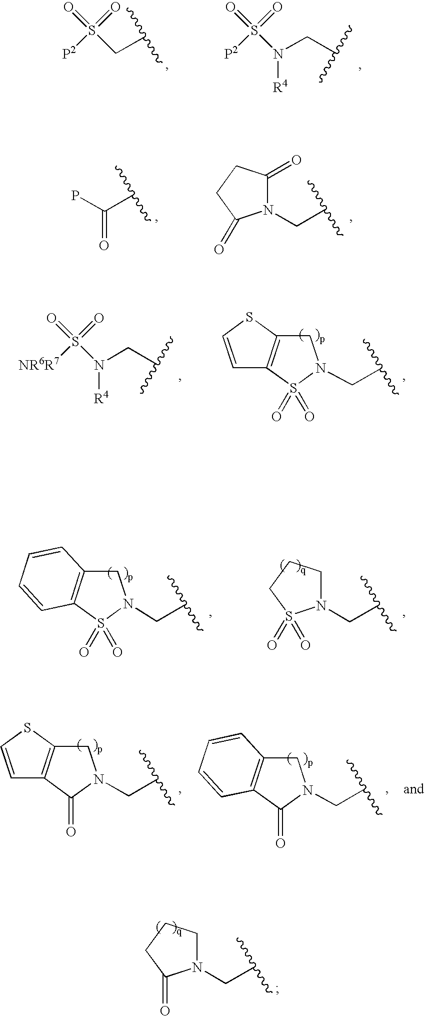

where p is 1 to 2, q is 1 to 3 and P² iS alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl,

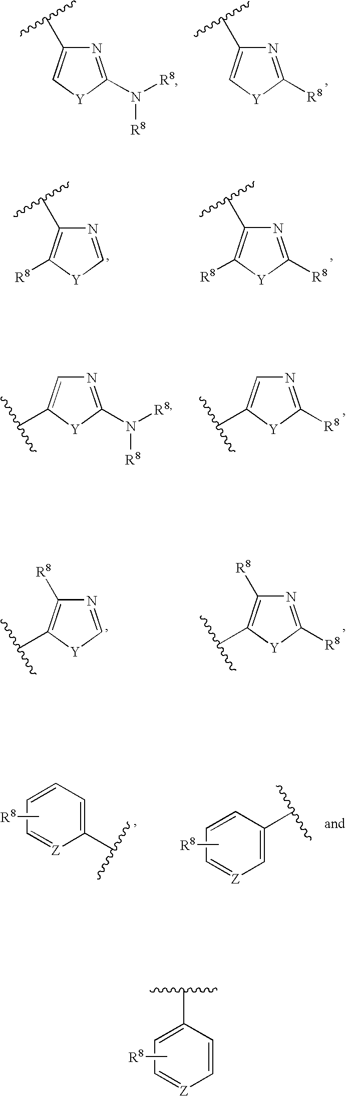

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the HCV protease inhibitor is a compound of Formula X: 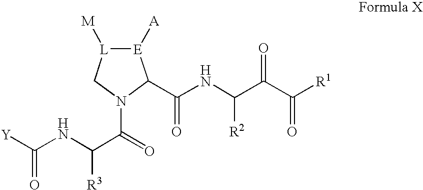
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 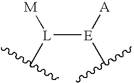
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties: 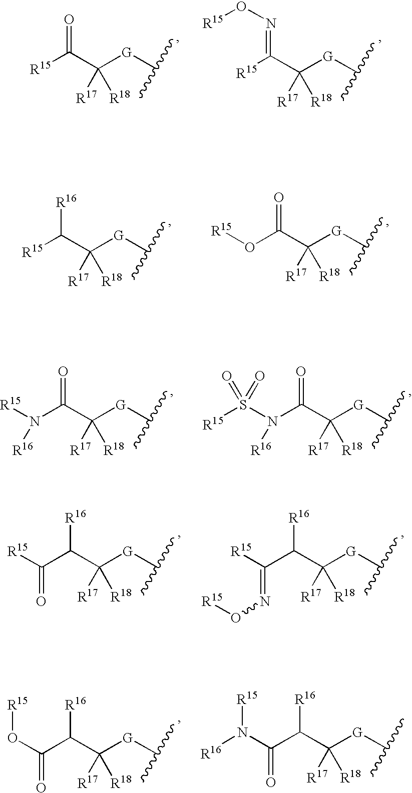
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In one embodiment, the HCV protease inhibitor is a compound of Formula XI: 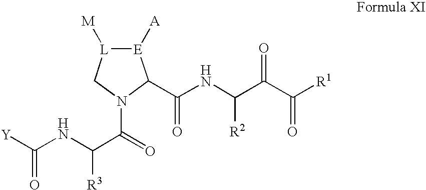
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, NR⁹R¹⁰, SR, SO₂R, and halo; or A and M are connected to each other (in other words, A-E-L-M taken together) such that the moiety: 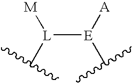
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;
Y is selected from the following moieties: 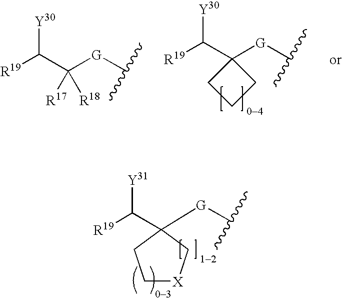
wherein Y³⁰ and Y³¹ are selected from 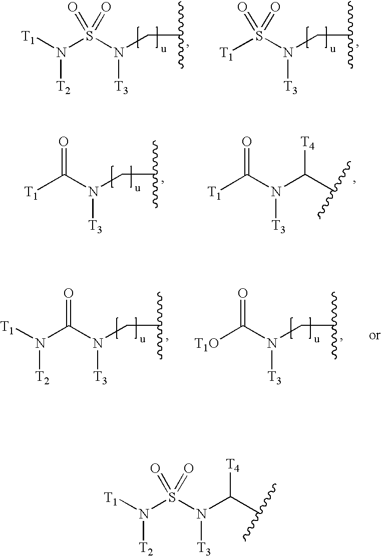
where u is a number 0-6.
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, T₃ and T₄ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XII: 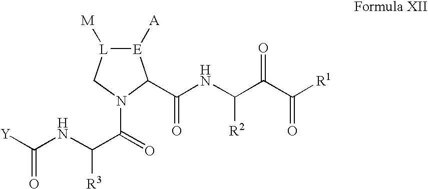
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 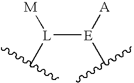
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties: 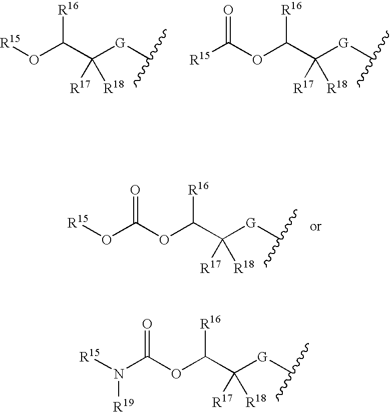
wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XIII: 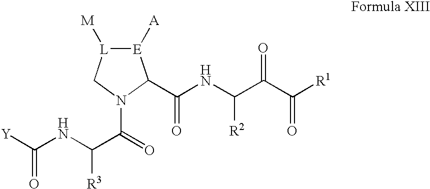
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other (in other words, A-E-L-M taken together) such that the moiety: 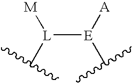
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 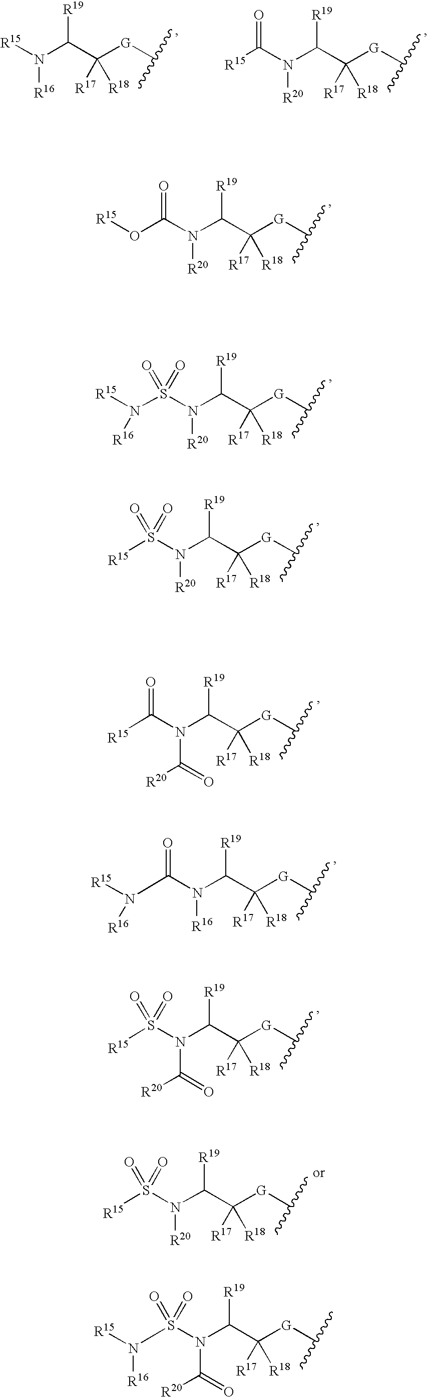
wherein G is NH or O, and R¹⁵, R¹⁶, R¹⁷, R¹⁸, R¹⁹ and R²⁰ can be the same or different, each being independently selected from the group consisting of H, C₁-C₁₀ alkyl, C_{1-C}₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XIV: 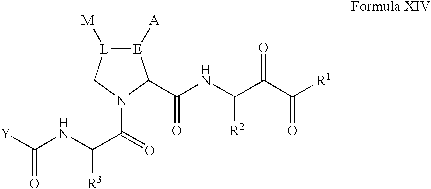
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo;
or A and M are connected to each other such that the moiety: 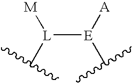
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;

and Y is selected from the following moieties: 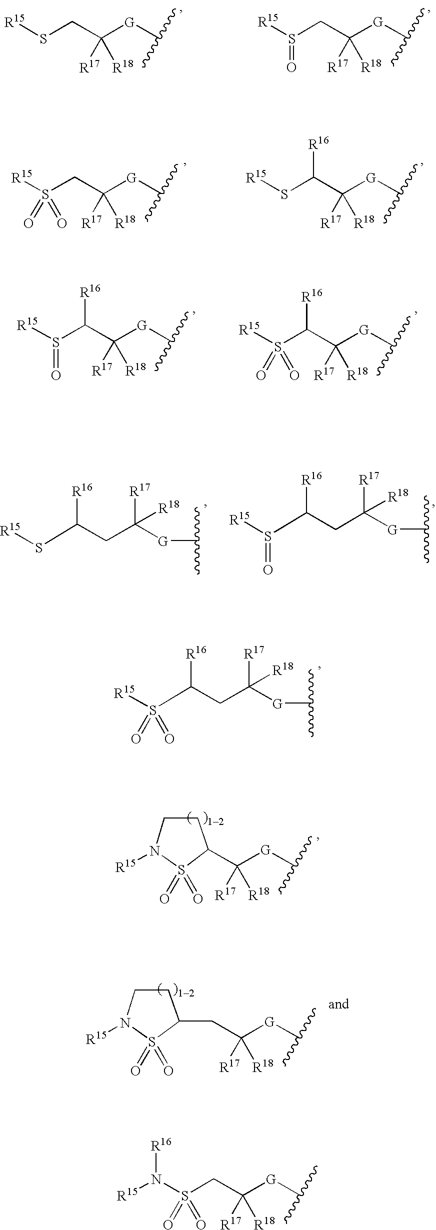
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XV: 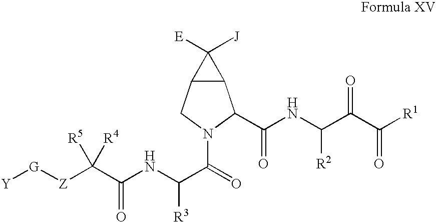
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, aryl-, heteroalkyl-, heteroaryl-, cycloalkyl-, cycloalkyl-, arylalkyl-, and heteroarylalkyl; E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;
Z is N(H), N(R), or O, with the proviso that when Z is O, G is present or absent and if
G is present with Z being O, then G is C(═O);
G maybe present or absent, and if G is present, G is C(═O) or S(O₂), and when G is absent, Z is directly connected to Y;

Y is selected from the group consisting of: 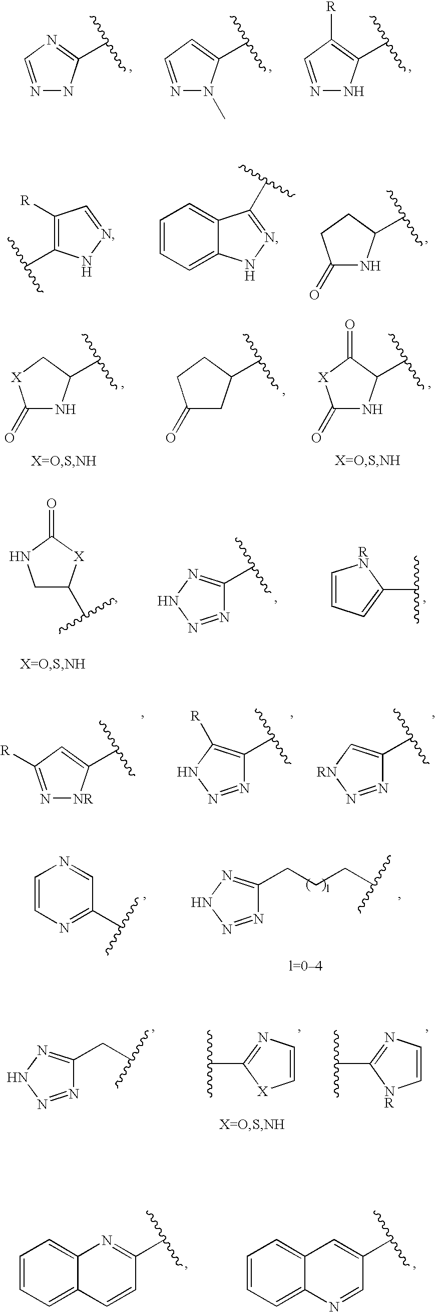 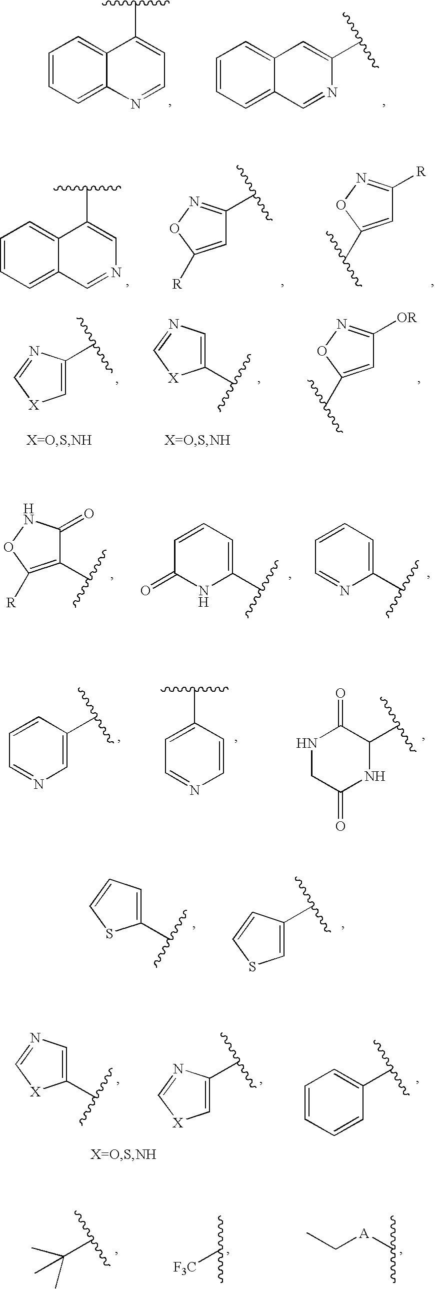 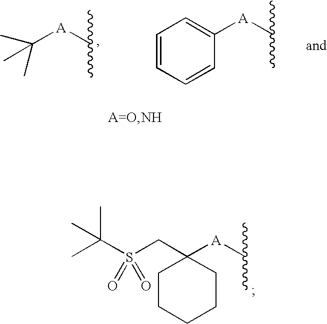
R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;
wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate.

In another embodiment, the HCV protease inhibitor is a compound of Formula XVI: 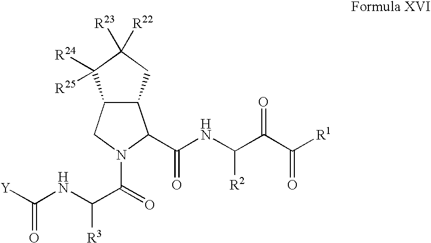
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties: 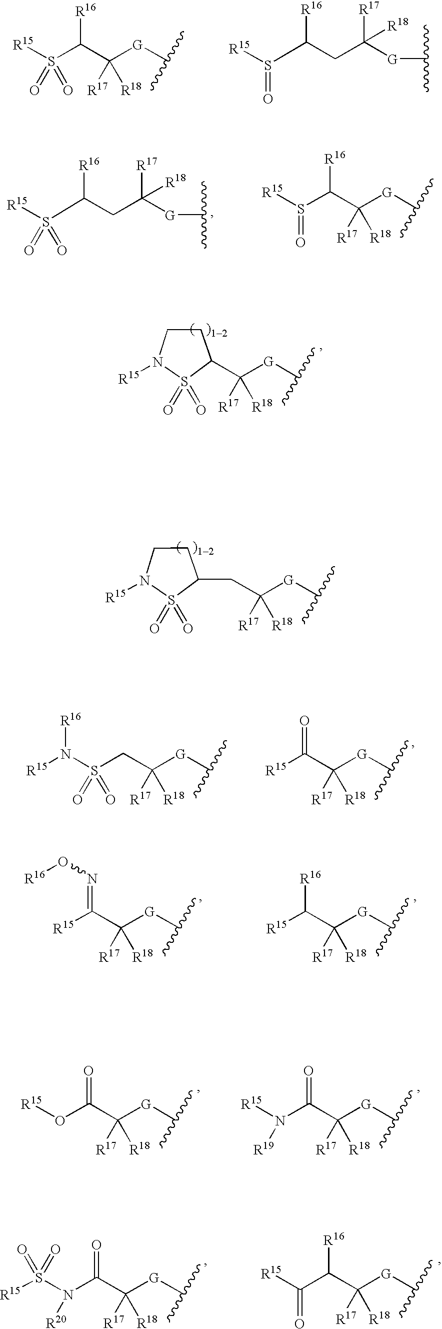 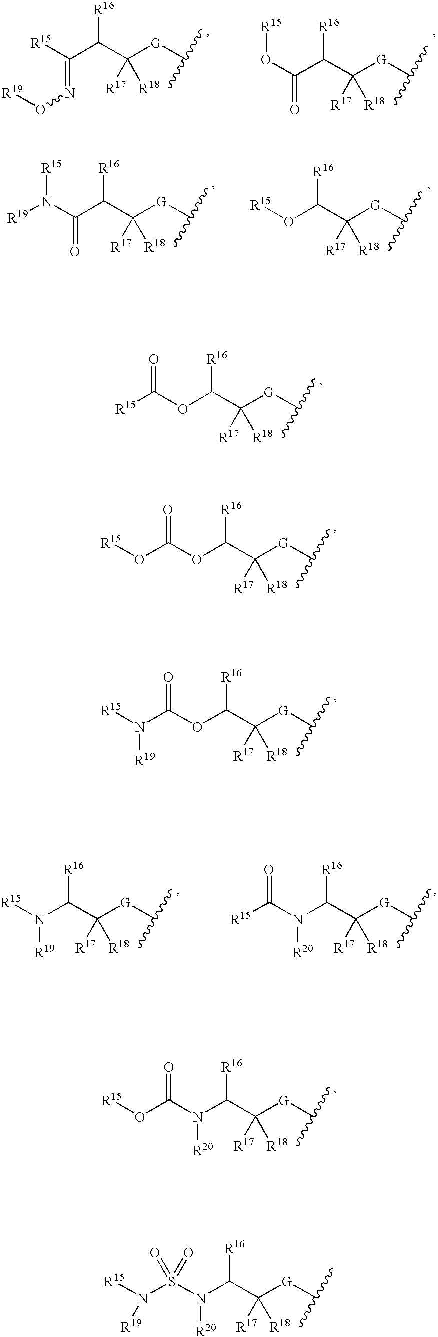 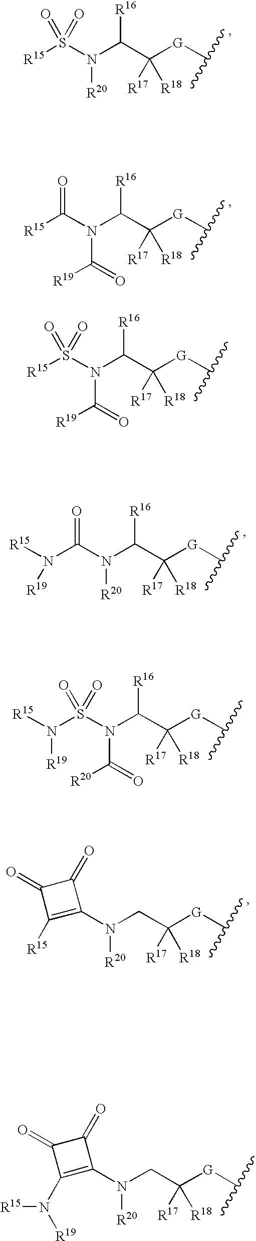
wherein G is NH or O; and R¹⁵, R¹⁶, R¹⁷, R¹⁸, R¹⁹R²⁰, R²¹, R²², R²³, R²⁴ and R²⁵ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XVII: 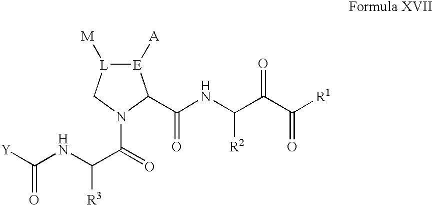
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 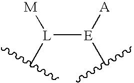
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
Y is selected from the following moieties: 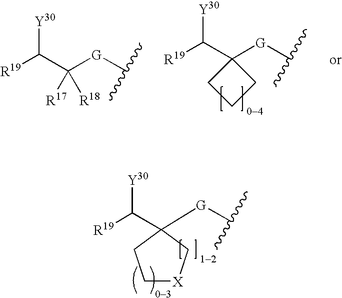
wherein Y³⁰ is selected from 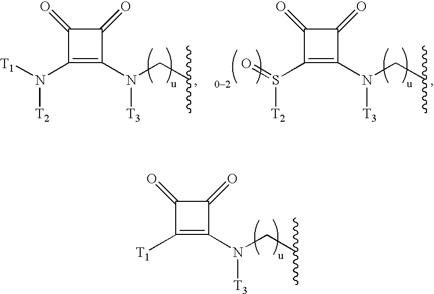
where u is a number 0-1;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XVIII: 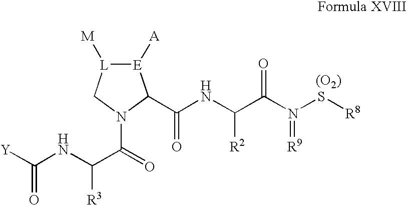
or a pharmaceutically acceptable salt, solvate or ester thereof, wherein:
R⁸ is selected from the group consisting of alkyl-, aryl-, heteroalkyl-, heteroaryl-, cycloalkyl-, heterocyclyl-, arylalkyl-, heteroarylalkyl- , and heterocyclylalkyl;
R⁹ is selected from the group consisting of H, alkyl, alkenyl, alkynyl, aryl and cycloalkyl;
A and M can be the same or different, each being independently selected from R, OR, N(H)R, N(RR′), SR, S(O₂)R, and halo; or A and M are connected to each other (in other words, A-E-L-M taken together) such that the moiety:

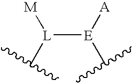

shown above in Formula I forms either a three, four, five, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl;
R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;

Y is selected from the following moieties: 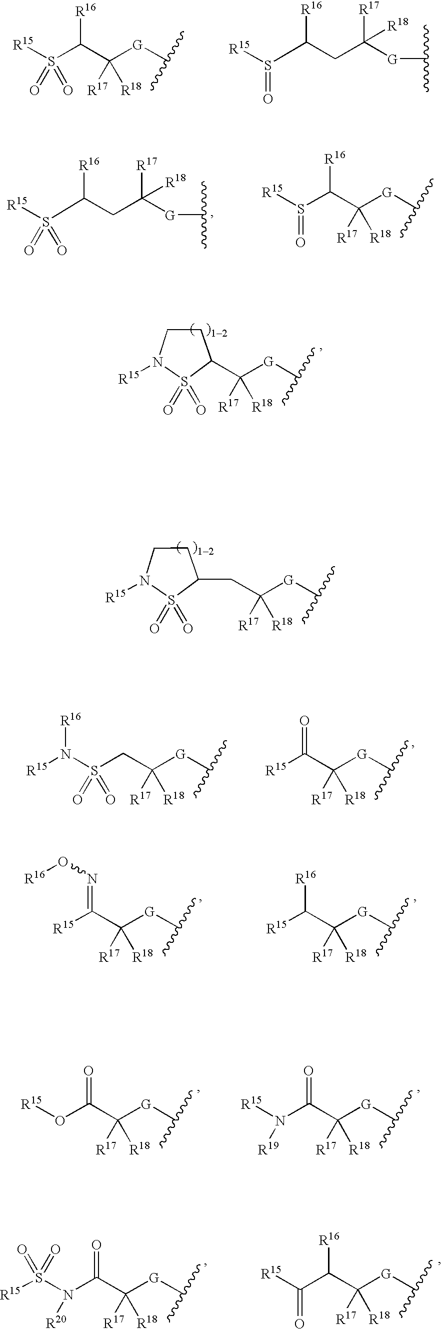 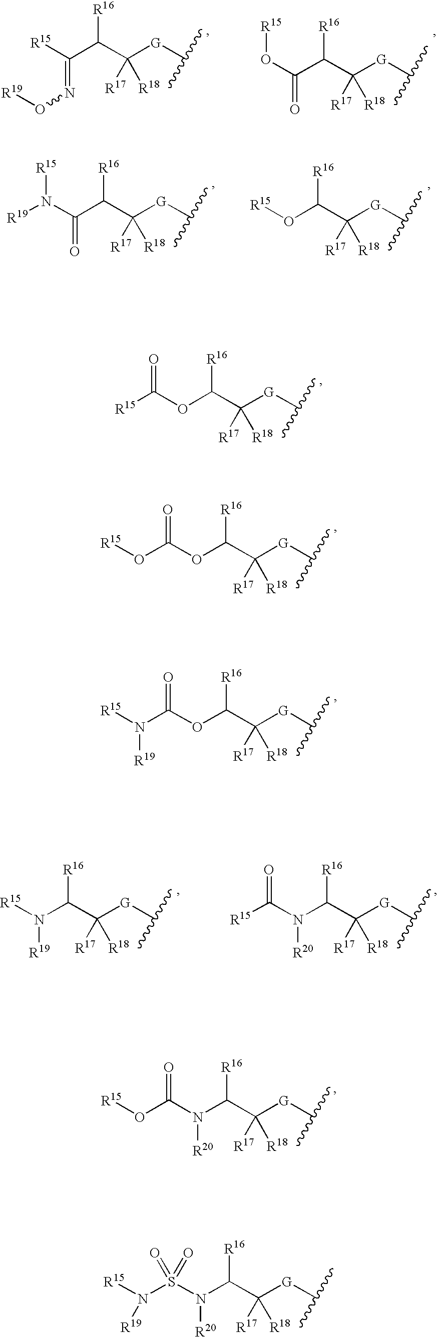 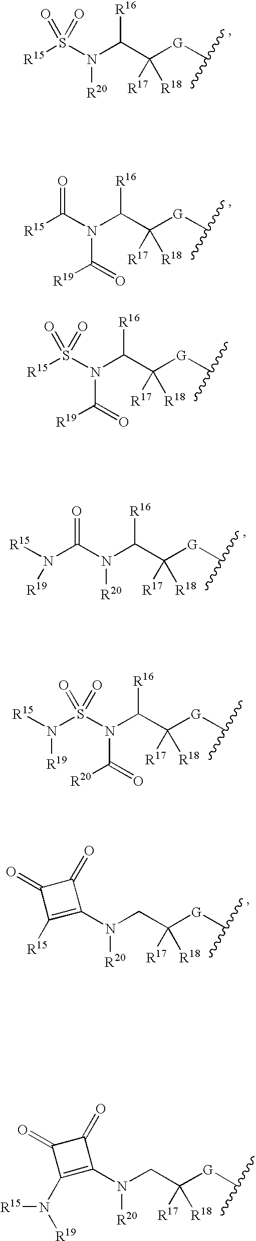
wherein G is NH or O; and R¹⁵, R¹⁶, R¹⁷, R¹⁸, R¹⁹and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl, spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XIX: 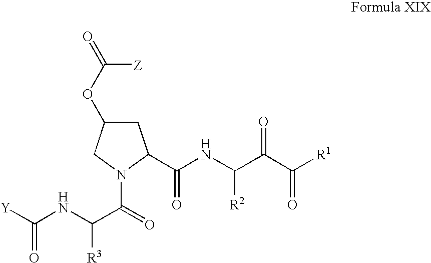
wherein:
Z is selected from the group consisting of a heterocyclyl moiety,

N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N(heteroaryl)₂;

R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties: 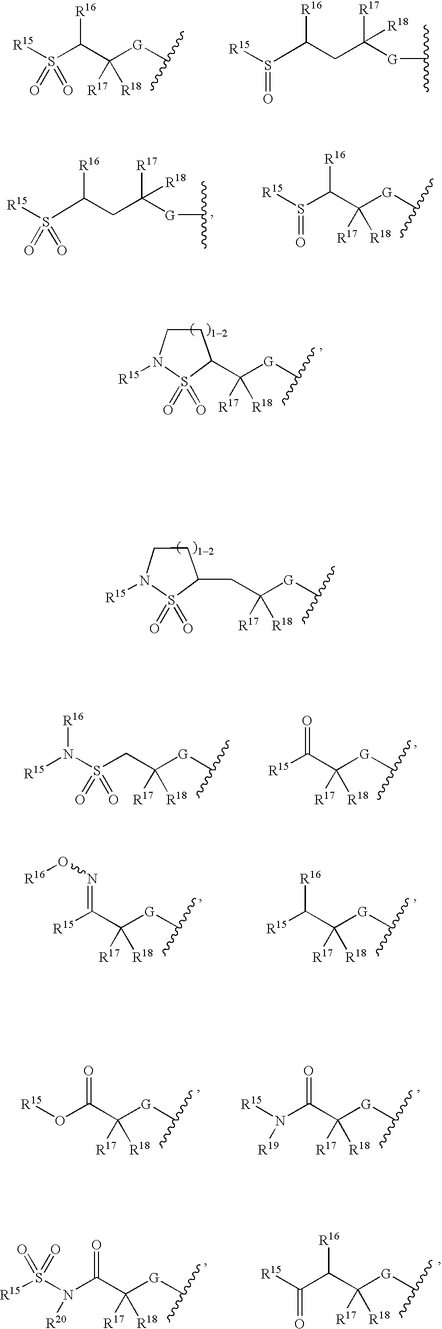 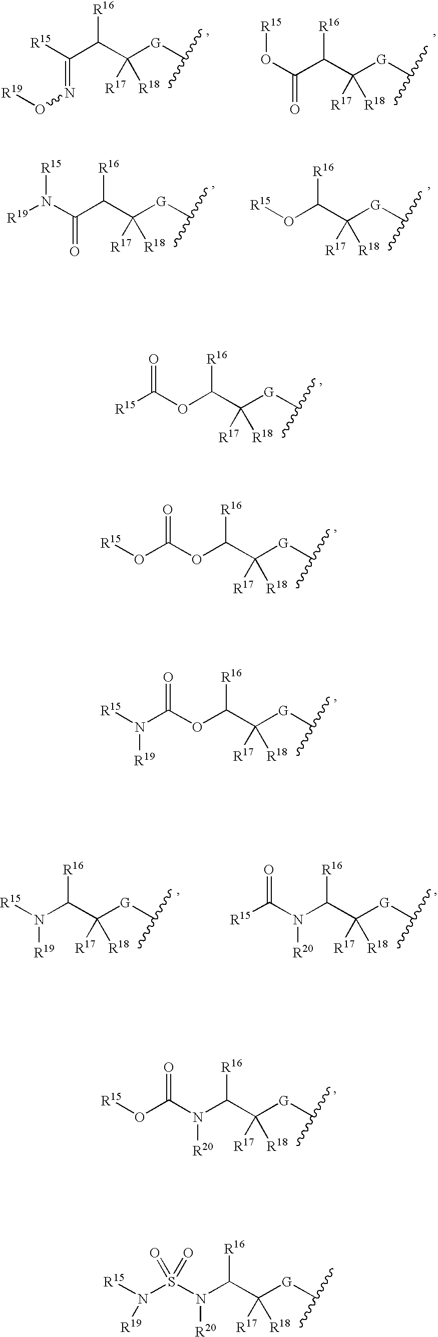 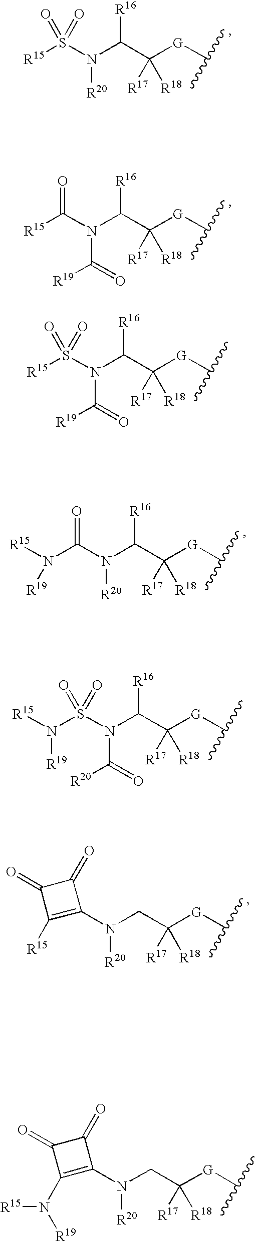
wherein G is NH or O; and R¹⁵, R¹⁶, R¹⁷, R¹⁸, R¹⁹, R²⁰ and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the HCV protease inhibitor is a compound of Formula XX 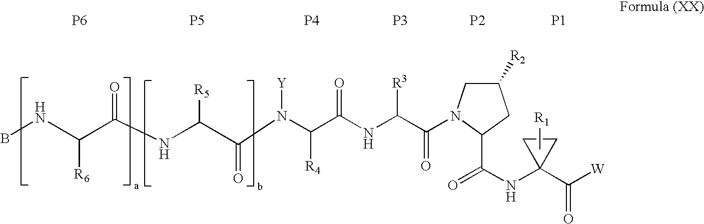
or a pharmaceutically acceptable salt, solvate or ester thereof; wherein: a is 0 or 1; b is 0 or 1; Y is H or C₁₋₆ alkyl;
B is H, an acyl derivative of formula R₇—C(O)— or a sulfonyl of formula R₇—SO2 wherein
R7 is (i) C_{I-10} alkyl optionally substituted with carboxyl, C₁₋₆ alkanoyloxy or C₁₋₆ alkoxy;
   (ii) C₃₋₇ cycloalkyl optionally substituted with carboxyl, (C_{I-6} alkoxy)carbonyl or phenylmethoxycarbonyl;
   (iii) C₆ or C₁₀ aryl or C₇₋₁₆ aralkyl optionally substituted with C₁₋₆ alkyl, hydroxy, or amino optionally substituted with C₁₋₆ alkyl; or
   (iv) Het optionally substituted with C₁₋₆ alkyl, hydroxy, amino optionally substituted with C₁₋₆ alkyl, or amido optionally substituted with C₁₋₆ alkyl;
R₆, when present, is C₁₋₆ alkyl substituted with carboxyl;
R₅, when present, is C₁₋₆ alkyl optionally substituted with carboxyl;
R₄ is C₁₋₁₀ alkyl, C₃₋₇ cycloalkyl or C₄₋₁₀ (alkylcycloalkyl);
R₃ is C₁₋₁₀ alkyl, C₃₋₇ cycloalkyl or C₄₋₁₀ (alkylcycloalkyl);
R₂ is CH₂-R₂₀, NH—R₂₀, O-R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri- substituted with R₂₁,
or R₂₀ is Het or (lower alkyl)-Het optionally mono-, di- or tri- substituted with R₂₁,
wherein each R₂₁ is independently C₁₋₆ alkyl; C₁₋₆alkoxy; amino optionally mono- or di-substituted with C₁₋₆ alkyl; sulfonyl; NO₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;
wherein R₂₂ is C₁₋₆alkyl; C₁₋₆ alkoxy; amino optionally mono- or di- substituted with C₁₋₆ alkyl; sulfonyl; NO₂; OH; SH; halo; haloalkyl; carboxyl; amide or (lower alkyl)amide;
R₁ is C₁₋₆ alkyl or C₂₋₆ alkenyl optionally substituted with halogen; and
W is hydroxy or a N-substituted amino.

In the above-shown structure of the compound of Formula XX, the terms P6, P5, P4, P3, P2 and P1 denote the respective amino acid moieties as is conventionally known to those skilled in the art.

In another embodiment, the HCV protease inhibitor is a compound of Formula XXI 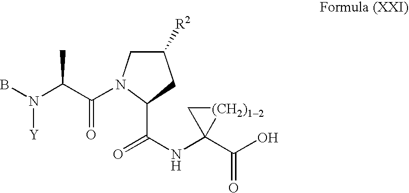
or a pharmaceutically acceptable salt, solvate or ester thereof; wherein:
B is H, a C₆ or C₁₀ aryl, C₇₋₁₆ aralkyl; Het or (lower alkyl)- Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl of formula R₄—O—C(O)—; an amide of formula R₄—N(R₅)—C(O)—; a thioamide of formula R₄—N(R₅)—C(S)—; or a sulfonyl of formula R₄—SO2 wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di- substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with C₁₋₆ alkyl;
R₅ is H or C₁₋₆ alkyl;
with the proviso that when R₄ is an amide or a thioamide, R₄ is not (ii) a cycloalkoxy;
Y is H or C₁₋₆ alkyl;
R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,
or R₂₀ is Het or (lower alkyl)-Het, both optionally mono-, di- or tri- substituted with R₂₁,
wherein each R₂₁ is independently C₁₋₆ alkyl; C₁₋₆ alkoxy; lower thioalkyl; sulfonyl; NO₂; OH; SH; halo; haloalkyl; amino optionally mono- or di- substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;
wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;
R1 is H; C₁₋₆ alkyl, C₃₋₇ cycloalkyl, C₂₋₆ alkenyl, or C₂₋₆ alkynyl, all optionally substituted with halogen.

In another embodiment, the HCV protease inhibitor is a compound of Formula XXII 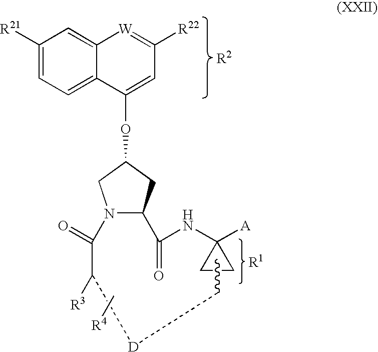
or a pharmaceutically acceptable salt, solvate or ester thereof; wherein W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂, wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C₆ or ₁₀ aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵, wherein each R²⁵ is independently: H, C₁₋₆ alkyl or C₃₋₆ cycloalkyl; or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹, wherein R⁴¹ is H, C₁₋₆ alkyl, C₃₋₆ cycloalkyl or —C(O)—R⁴², wherein R⁴² is C₁₋₆ alkyl, C₃₋₆ cycloalkyl or C_{6 or 10} aryl; R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C1-6 thioalkyl, and A is an amide of formula —C(O)—NH—R⁵, wherein R⁵ is selected from the group consisting of: C₁₋₈ alkyl, C₃₋₆ cycloalkyl, C_{6 or 10} aryl and C₇₋₁₆ aralkyl;
or A is a carboxylic acid.

In another embodiment, the HCV protease inhibitor is a compound of Formula XXIII 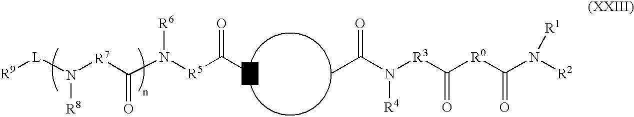
a pharmaceutically acceptable salt, solvate or ester thereof; wherein:
R⁰ is a bond or difluoromethylene;
R¹ is hydrogen, optionally substituted aliphatic group, optionally substituted cyclic group or optionally substituted aromatic group;
R² and R⁹ are each independently optionally substituted aliphatic group, optionally substituted cyclic group or optionally substituted aromatic group;
R3, R5 and R7 are each independently:

optionally substituted (1,1- or 1,2-)cycloalkylene; or

optionally substituted (1,1- or 1,2-) heterocyclylene; or

methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or;
R4, R6, R8 and R¹⁰ are each independently hydrogen or optionally substituted aliphatic group; 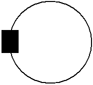
is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-(N(R⁸)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰C(O)—, —S(O)₂—, or —NR¹⁰S(O)₂—; and n is 0 or 1, provided when 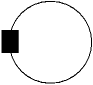
is substituted 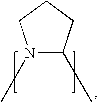
then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴ is optionally substituted aliphatic.

In another embodiment, the HCV protease inhibitor is a compound of Formula (XXIV) 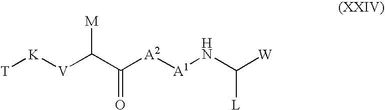
or a pharmaceutically acceptable salt, solvate or ester thereof; wherein:
W is:

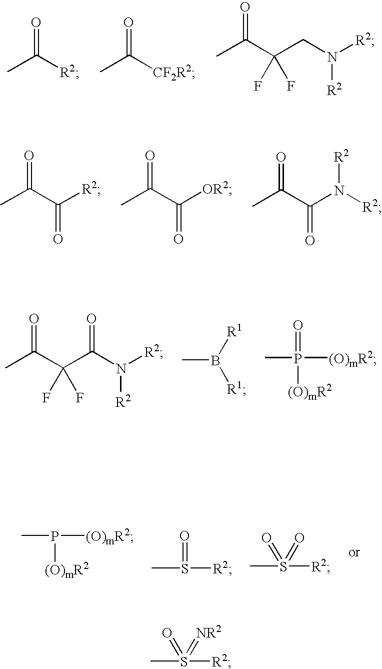

m is 0 or 1;

each R¹ is hydroxy, alkoxy, or aryloxy, or each R¹ is an oxygen atom and together with the boron, to which they are each bound, form a 5-7 membered ring, wherein the ring atoms are carbon, nitrogen, or oxygen;

each R² is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl, or two R² groups, which are bound to the same nitrogen atom, form together with that nitrogen atom, a 5-7 membered monocyclic heterocyclic ring system; wherein any R² carbon atom is optionally substituted with J;

J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;

J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoaikyl, halo, cyano, nitro, formyl, sulfonyl, or sulfonamido;

L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;

A¹ is a bond;

R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;

X is a bond, —C(H)(R7)—, —O—, —S—, or —N(R8)—;

R⁷ is hydrogen, alkyl, alkenyl, aryl, aralkyl, heterocyclyl, heterocyclylalkyl, heteroaryl, or heteroaralkyl, and is optionally substititued with 1-3 J groups;

R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R¹⁴, —SO₂R¹⁴, or carboxamido, and is optionally substititued with 1-3 J groups; or R⁸ and Z, together with the atoms to which they are bound, form a nitrogen containing mono- or bicyclic ring system optionally substituted with 1-3 J groups;

R¹⁴ is alkyl, aryl, aralkyl, heterocyclyl, heterocyclyalkyl, heteroaryl, or heteroaralkyl;

Y is a bond, —CH₂—, —C(O)—, —C(O)C(O)—, —S(O)—, —S(O)₂—, or —S(O)(NR⁷)—, wherein R⁷ is as defined above;

Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;

A² is a bond or

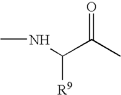

R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;

V is a bond, —CH₂—, —C(H)(R¹¹)—, —O—, —S—, or —N(R¹¹)—;

R¹¹ is hydrogen or C₁₋₃ alkyl;

K is a bond, —O—, —S—, —C(O)—, —S(O)—, —S(O)₂—, or —S(O)(NR¹¹)—, wherein R¹¹ is as defined above;

T is —R¹², -alkyl-R¹², -alkenyl-R¹², - alkynyl-R¹², —OR¹², —N(R¹²)2, —C(O)R¹², —C(═NOalkyl)R¹², or

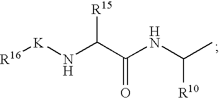

R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;

R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;

R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and

R¹⁶ is hydrogen, alkyl, aryl, heteroaryl, cycloalkyl, or heterocyclyl.

In another embodiment, the HCV protease inhibitor is a compound of Formula XXV 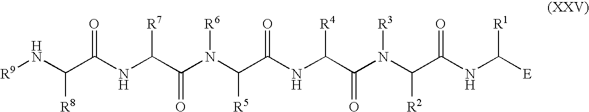
or a pharmaceutically acceptable salt, solvate or ester thereof;
wherein

E represents CHO or B(OH)2;

R¹ represents lower alkyl, halo-lower alkyl, cyano-lower alkyl, lower alkylthio-lower alkyl, aryl-lower alkylthio-lower alkyl, aryl-lower alkyl, heteroaryllower alkyl, lower alkenyl or lower alkynyl;

R² represents lower alkyl, hydroxy-lower alkyl, carboxylower alkyl, aryl- lower alkyl, aminocarbonyl-lower alkyl or lower cycloalkyl-lower alkyl; and

R³ represents hydrogen or lower alkyl;

or R² and R³ together represent di- or trimethylene optionally substituted by hydroxy;

R⁴ represents lower alkyl, hydroxy-lower alkyl, lower cycloalkyl-lower alkyl, carboxy-lower alkyl, aryllower alkyl, lower alkylthio-lower alkyl, cyano-lower alkylthio-lower alkyl, aryl-lower alkylthio-lower alkyl, lower alkenyl, aryl or lower cycloalkyl;

R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;

R⁶ represents hydrogen or lower alkyl;

R⁷ represent lower alkyl, hydroxydower alkyl, carboxylower alkyl, aryl-iower alkyl, lower cycloalkyl-lower alkyl or lower cycloalkyl;

R⁸ represents lower alkyl, hydroxy-lower alkyl, carboxylower alkyl or aryl-lower alkyl; and

R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl.

In another embodiment, the HCV protease inhibitor is a compound of Formula XXVI 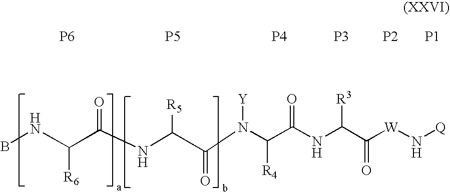
or a pharmaceutically acceptable salt, solvate or ester thereof; wherein

B is an acyl derivative of formula R₁₁—C(O)— wherein R₁₁ is Cl-10 alkyl optionally substituted with carboxyl; or R₁₁ is C₆ or C₁₀ aryl or C₇₋₁₆ aralkyl optionally substituted with a C₁₋₆ alkyl;

a is 0 or 1;

R₆, when present, is carboxy(lower)alkyl;

b is 0 or 1;

R₅, when present, is C₁₋₆ alkyl, or carboxy(lower)alkyl;

Y is H or C₁₋₆ alkyl;

R₄ is C₁₋₁₀ alkyl; C₃₋₁₀ cycloalkyl;

R₃ is C1-10 alkyl; C₃₋₁₀ cycloalkyl;

W is a group of formula:

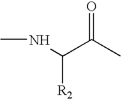

wherein R₂ is C₁₋₁₀ alkyl or C₃₋₇ cycloalkyl optionally substituted with carboxyl; C₆ or C₁₀ aryl; or C₇₋₁₆ aralkyl; or

W is a group of formula:

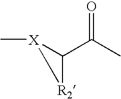

wherein X is CH or N; and

R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:

cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: O, S, and N; or

R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: O, S, and N;

said cyclic alkyl, cyclic alkenyl, aryl or aralkyl being optionally fused with a second 5-, 6-, or 7-membered ring to form a cyclic system or heterocycle, said second ring being optionally substituted with NH₂. OH, SH, halo, carboxyl or carboxy(lower)alkyl; C₆ or C₁₀ aryl, or heterocycle; said second ring optionally containing at least one heteroatom selected independently from the group consisting of: O, S, and N;

Q is a group of the formula:

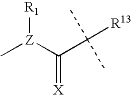

wherein Z is CH or N;

X is O or S;

R₁ is H, C₁₋₆ alkyl or C₁₋₆ alkenyl both optionally substituted with thio or halo; and

when Z is CH, then R₁₃ is H; CF₃; CF₂CF₃; CH₂—R₁₄; CH(F)—R₁₄; CF₂—R₁₄; NR₁₄R₁₄′; S—R₁₄; or CO—NH—R₁₄ wherein R₁₄ and R₁₄′ are independently hydrogen, cyclic C₃₋₁₀ alkyl or acyclic C₁₋₁₀ alkyl or cyclic C₃₋₁₀ alkenyl or acyclic C₂₋₁₀ alkenyl, said alkyl or alkenyl optionally substituted with NH₂, OH, SH, halo or carboxyl; said alkyl or alkenyl optionally containing at least one heteroatom selected independently from the group consisting of: O, S, and N; or

R₁₄ and R₁₄′ are independently C₆ or C₁₀ aryl or C₁₋₆ aralkyl optionally substituted with C16 alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: O, S, and N;

said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: O, S, and N;

or R₁₄ and R₁₄′ are independently C₁₋₄ alkyl which when joined together with N form a 3 to 6-membered nitrogen-containing ring which is optionally fused with a further C₃₋₇ cycloalkyl, C₆ or C₁₀ aryl or heterocycle;

with the proviso that when Z is CH, then R₁₃ is not an α-amino acid or an ester thereof;

when Z is N, then R₁₃ is H; carboxy; C₁₋₆ alkyl optionally substituted with carboxy; CH₂-R₁₄; CHR₁₄R₁₄′; CH(F)-R₁₄; O—R₁₄; NR₁₄R₁₄′ or S—R₁₄ wherein R₁₄ and R₁₄′ are as defined above; or

Q is a phosphonate group of the formula:

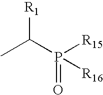

wherein R₁₅ and R₁₆ are independently C₆₋₂₀ aryloxy; and R₁ is as defined above.

In the above-shown structure of the compound of Formula XXVI, the terms P6, P5, P4, P3, P2 and P1 denote the respective amino acid moieties as is conventionally known to those skilled in the art. Thus, the actual structure of the compound of Formula XXVI is:

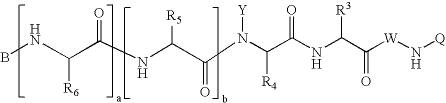

In another embodiment, the compound is selected from the group consisting of: 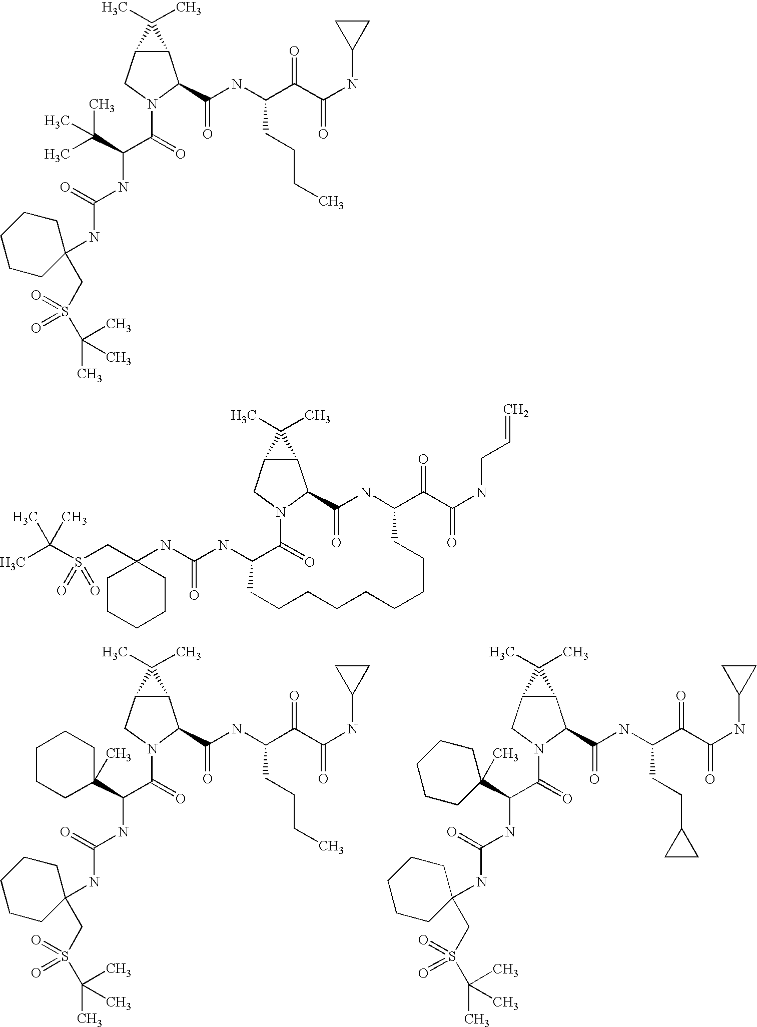 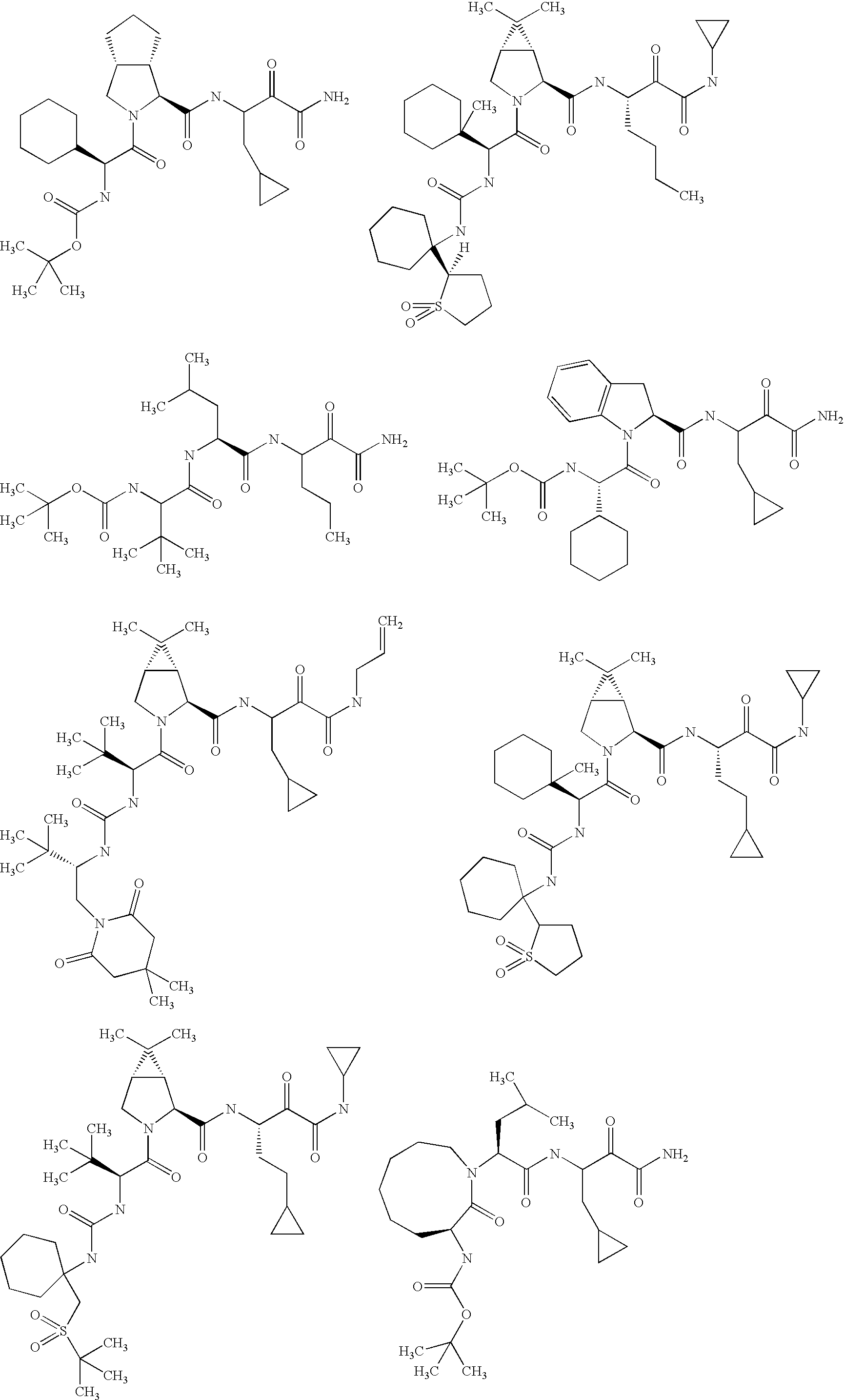 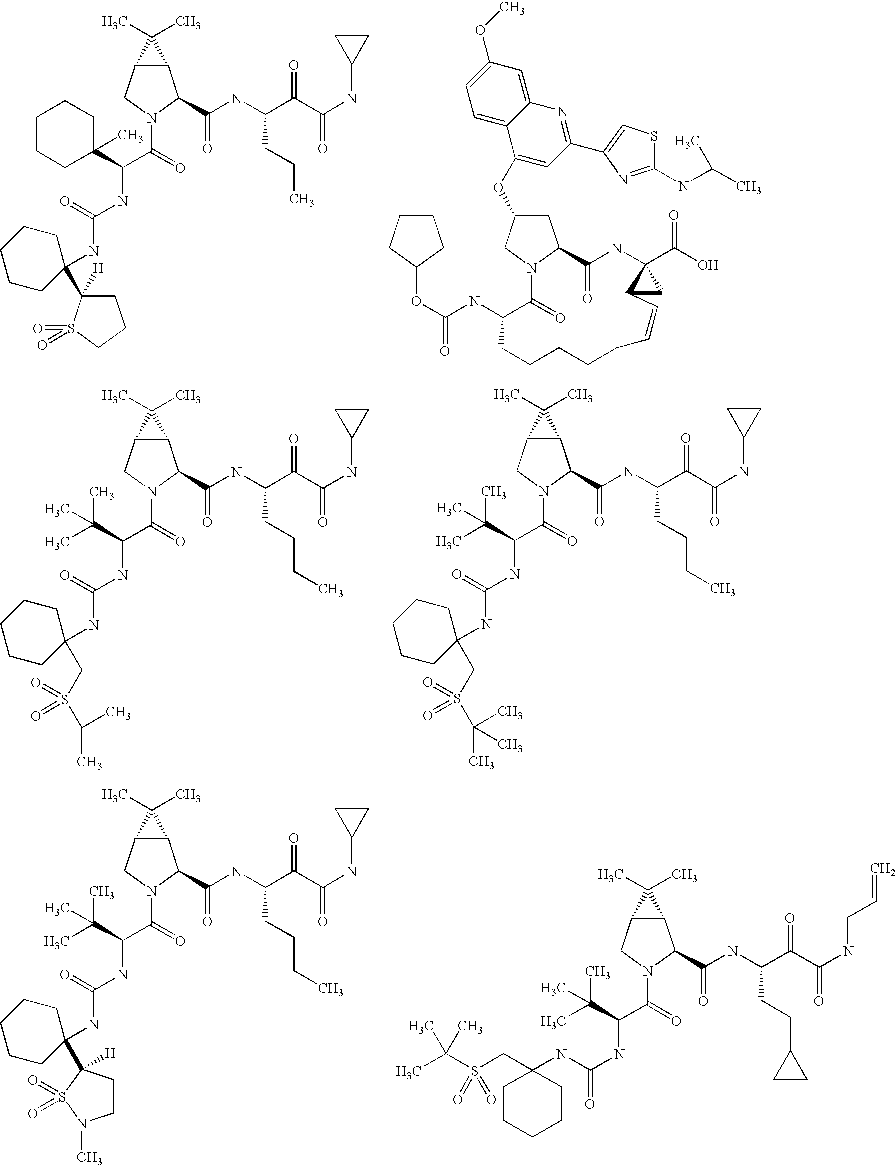 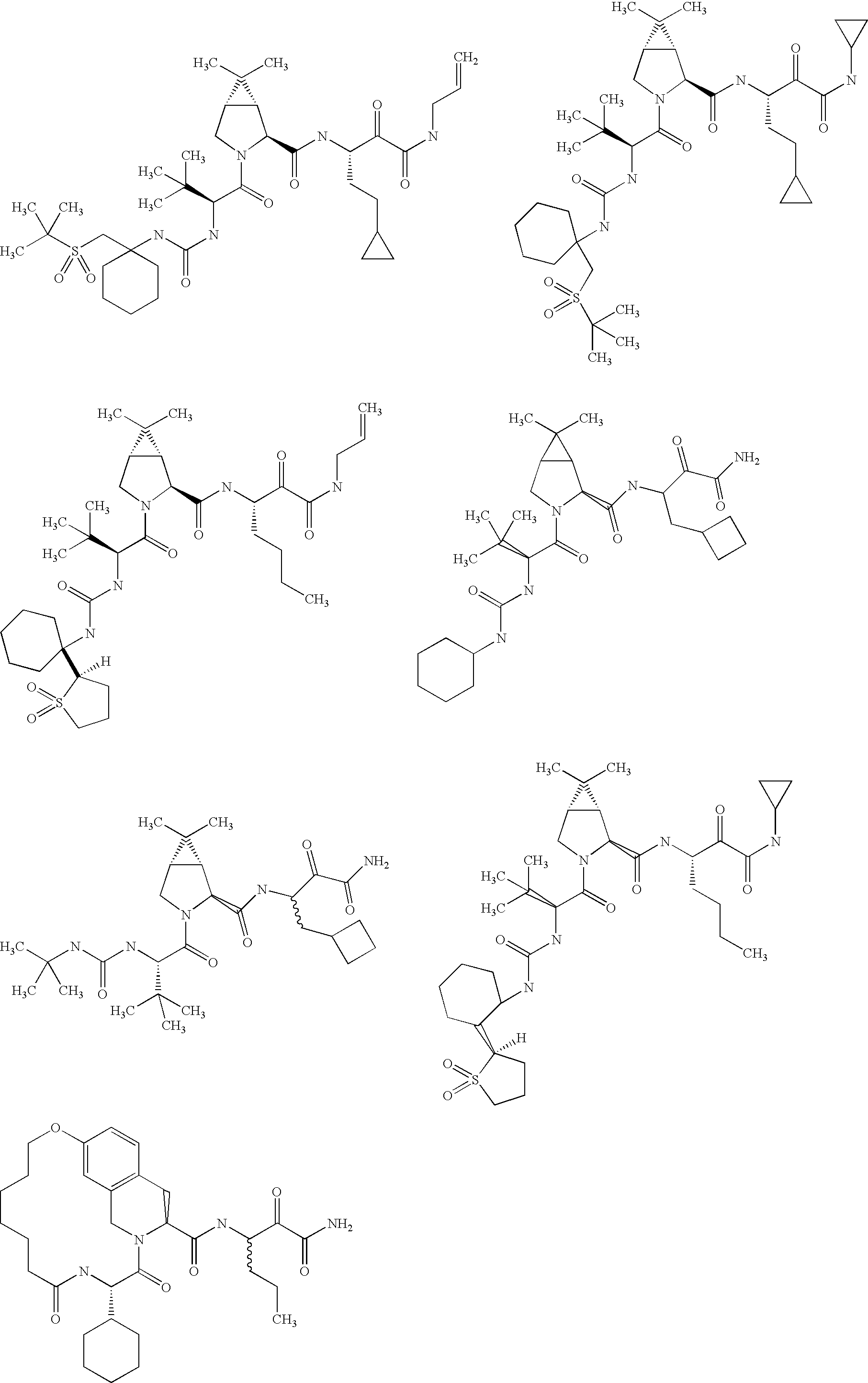 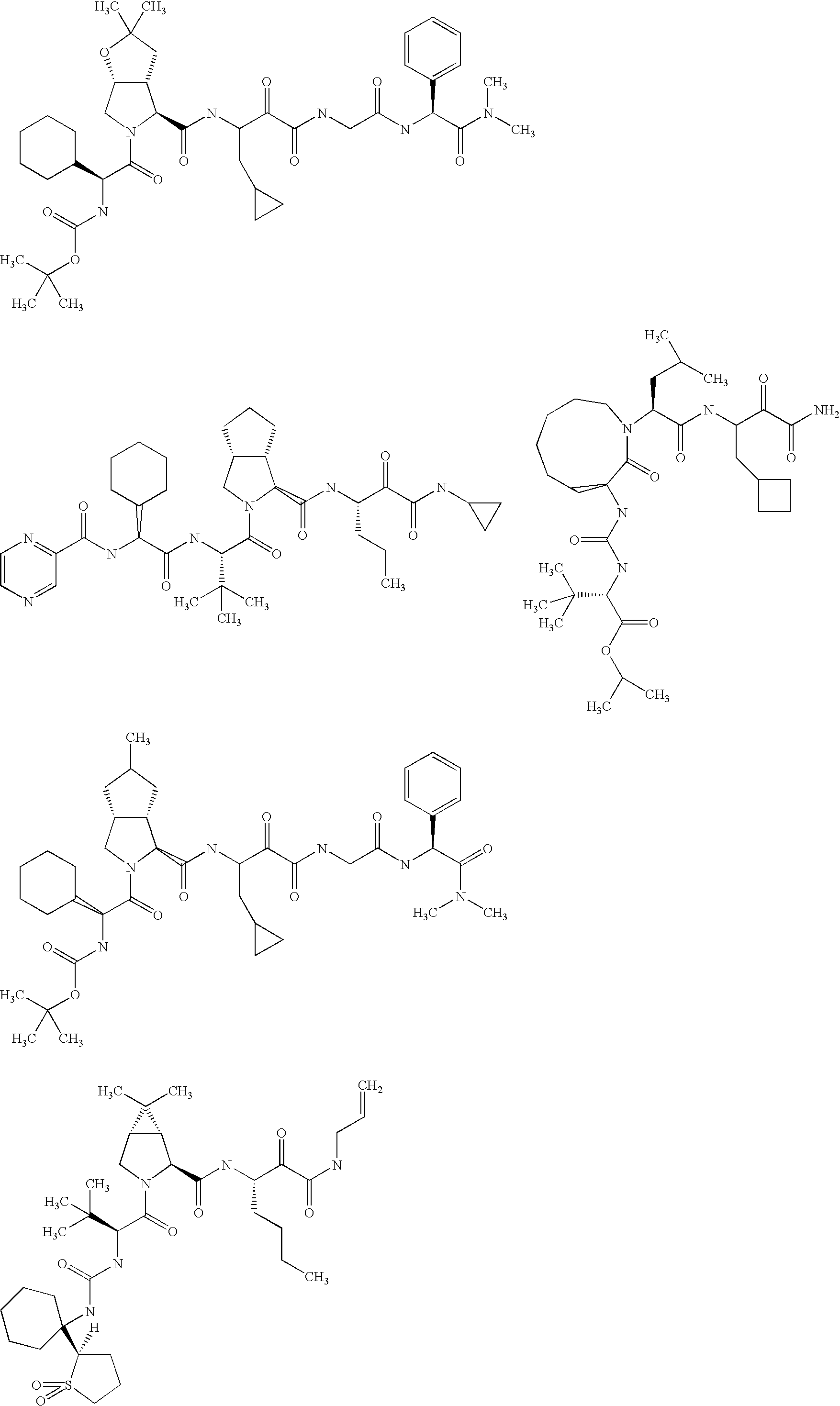 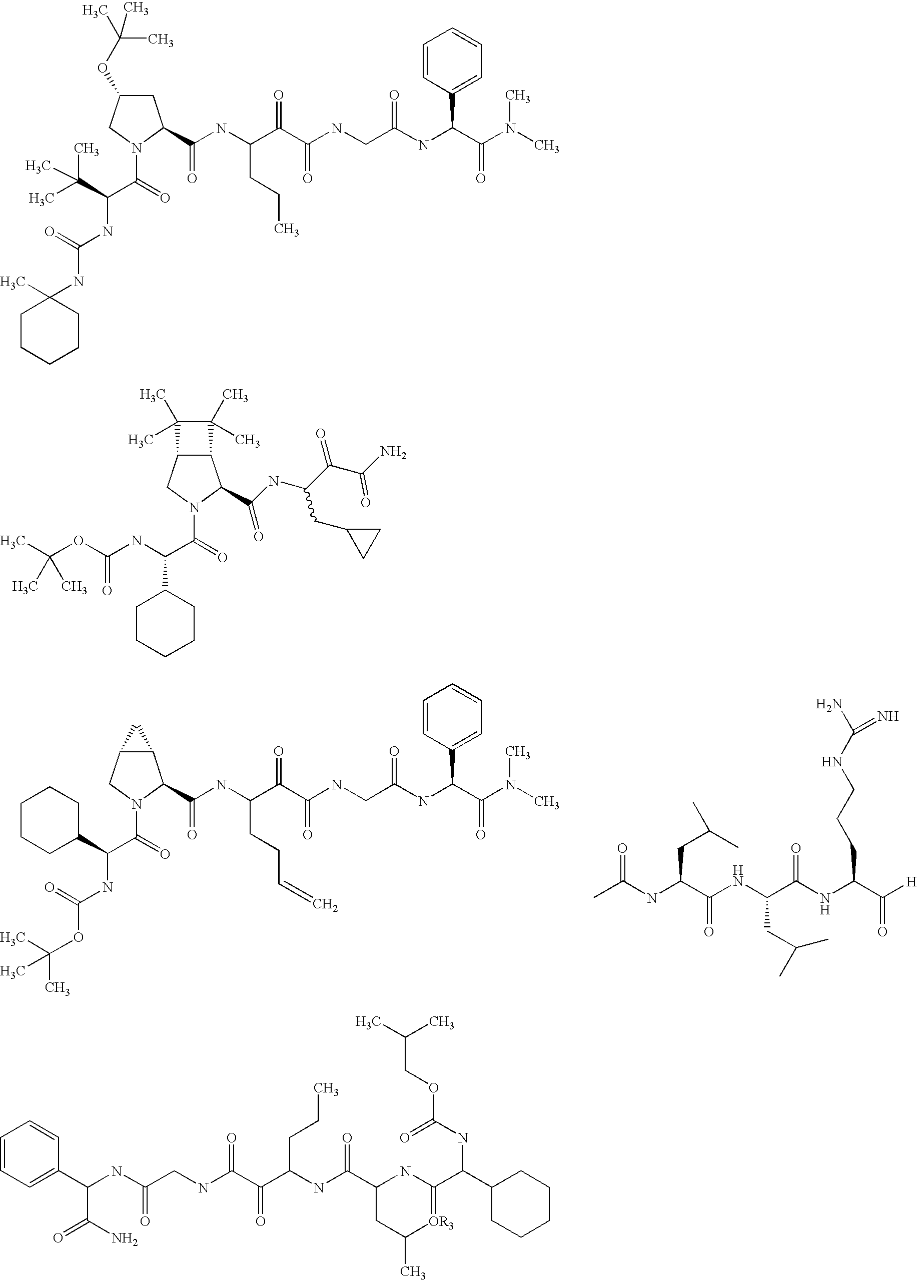 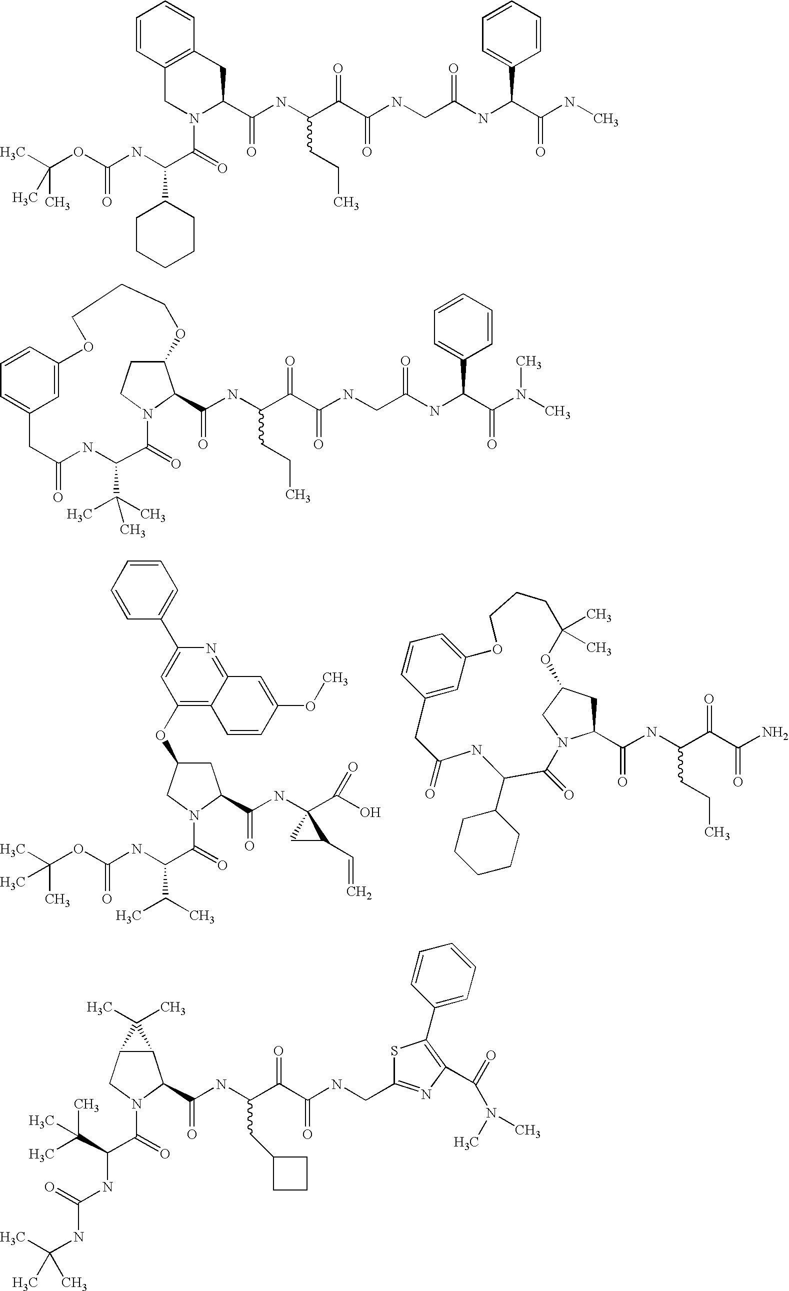 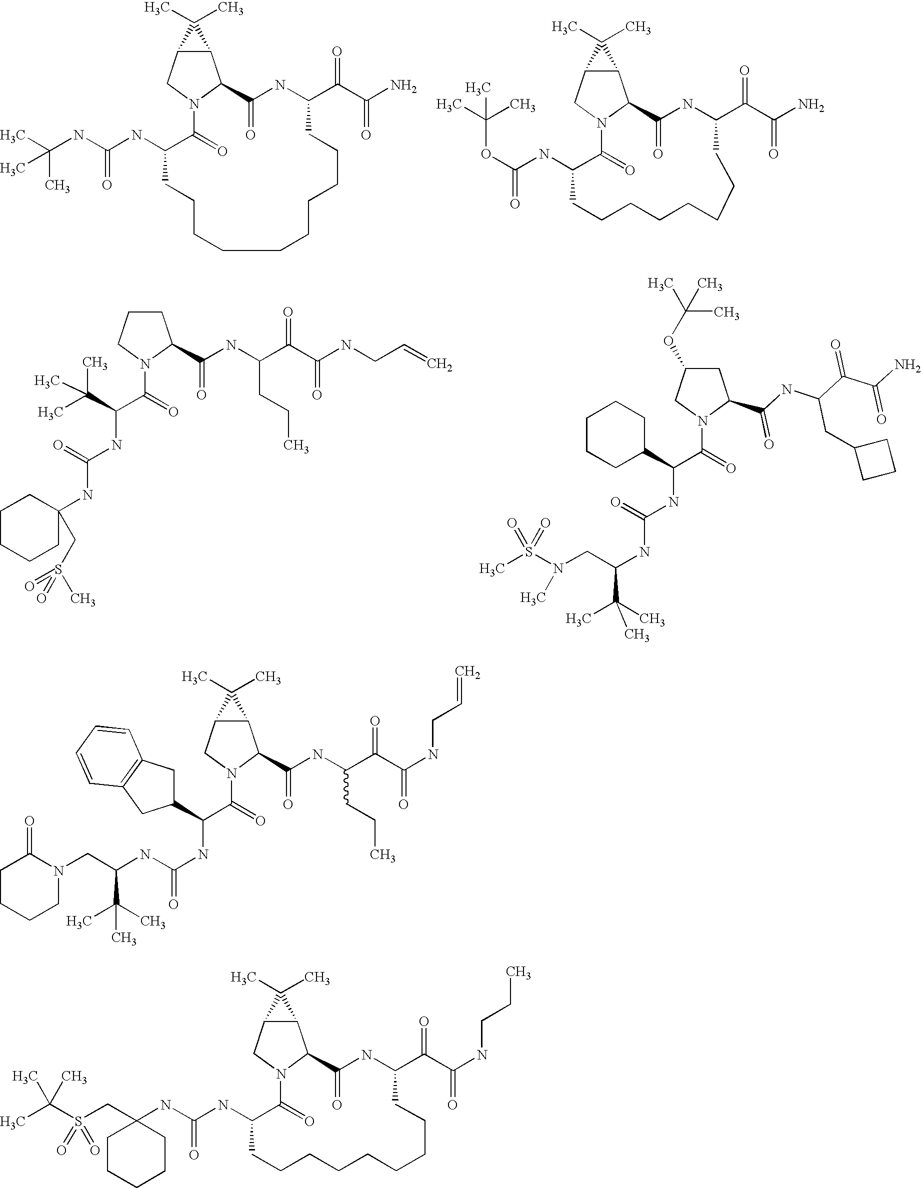 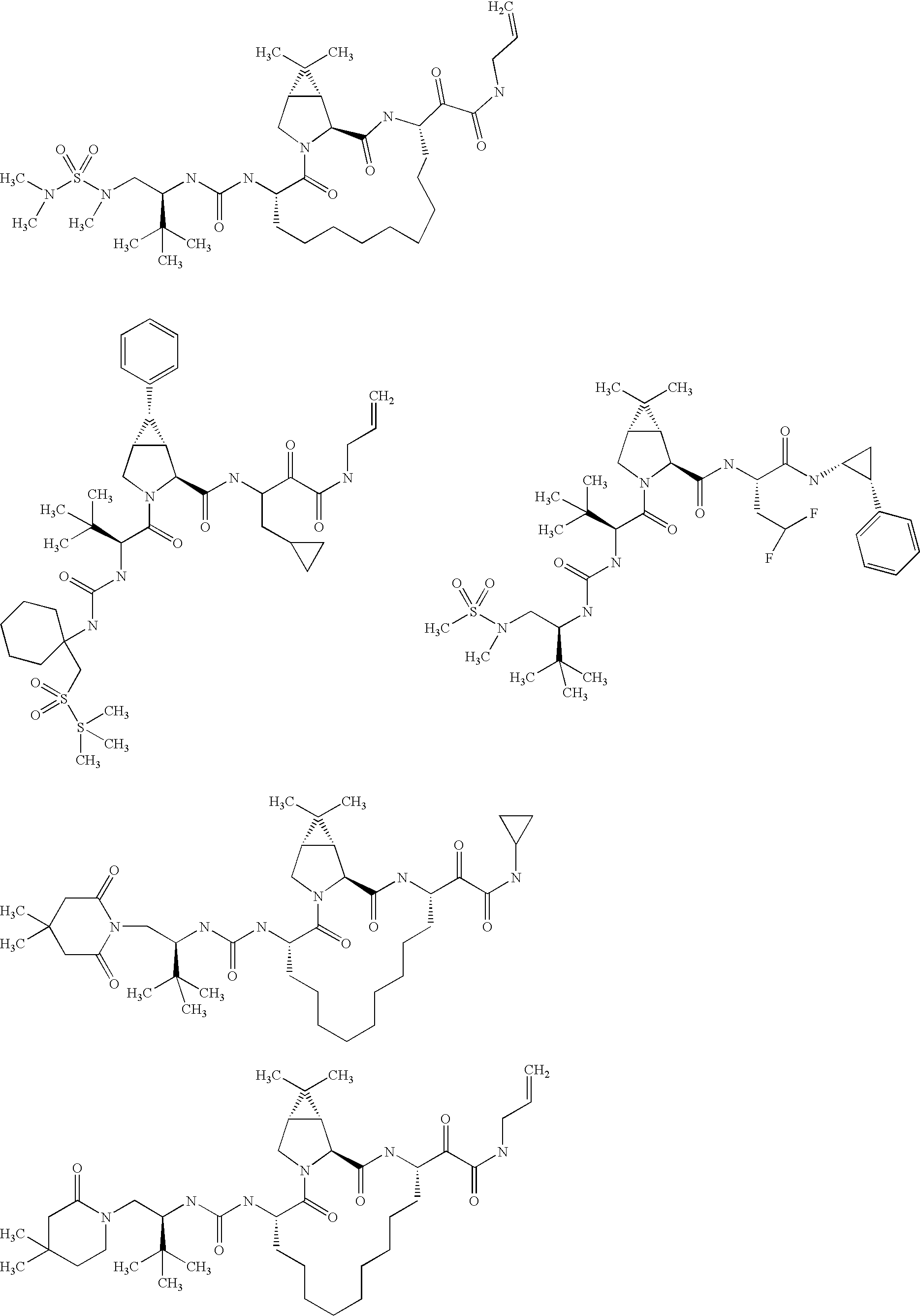 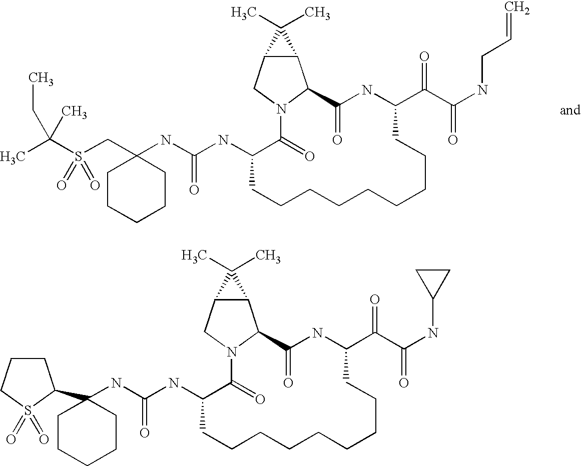
or a pharmaceutically acceptable salt, solvate or ester thereof.

Methods of treating a wide variety of diseases/disorders associated with cathepsin activity and/or for inhibiting cathepsin activity in a subject comprising administering to a subject in need of such treatment an effective amount of at least one of the inventive compounds also are provided.

One example of such disorders is proliferative diseases, such as cancer, autoimmune diseases, viral diseases, fungal diseases, neurological/neurodegenerative disorders, arthritis, inflammation, anti-proliferative (e.g., ocular retinopathy), neuronal, alopecia and cardiovascular disease. Many of these diseases and disorders are listed in U.S. Pat. No. 6,413,974, the disclosure of which is incorporated herein.

Another example of a disease that can be treated by the present compounds is an inflammatory disease, such as organ transplant rejection, graft v. host disease, arthritis, rheumatoid arthritis, inflammatory bowel disease, atopic dermatitis, psoriasis, asthma, allergies, multiple sclerosis, fixed drug eruptions, cutaneous delayed-type hypersentitivity responses, tuberculoid leprosy, type I diabetes, and viral meningitis.

Another example of a disease that can be treated by the present compounds is a cardiovascular disease.

Another example of a disease that can be treated by the present compounds is a central nervous system disease, such as depression, cognitive function disease, neurodegenerative disease such as Parkinson's disease, senile dementia such as Alzheimer's disease, and psychosis of organic origin.

Other examples of diseases that can be treated by the present compounds are diseases characterized by bone loss, such as osteoporosis; gingival diseases, such as gingivitis and periodontitis; and diseases characterized by excessive cartilage or matrix degradation, such as osteoarthritis and rheumatoid arthritis.

Other than in the operating examples, or where otherwise indicated, all numbers expressing quantities of ingredients, reaction conditions, and so forth used in the specification and claims are to be understood as being modified in all instances by the term “about.”

### BRIEF DESCRIPTION OF THE DRAWINGS

The invention is further illustrated by the following drawings in which:

FIG. 1 is a graph of mean viral load measured over time with administration of various doses and regimens of a compound of Formula Ia; and

FIG. 2 is a box plot showing trough plasma levels of a compound of Formula Ia measured on day 14 at various times when a compound of Formula Ia is administered twice per day (bid, left box) and when a compound of Formula Ia is administered three times per day (tid, right box).

### DETAILED DESCRIPTION OF THE INVENTION

The present invention is directed to methods of treatment capable of maintaining an average plasma Cmin of at least one (one or more) compounds of Formulae I to XXVI at or above about 10 ng/ml in a subject.

Suitable compounds of formula I are disclosed in PCT International publication WO03/062265 published Jul. 31, 2003. Non-limiting examples of certain compounds disclosed in this publication include: 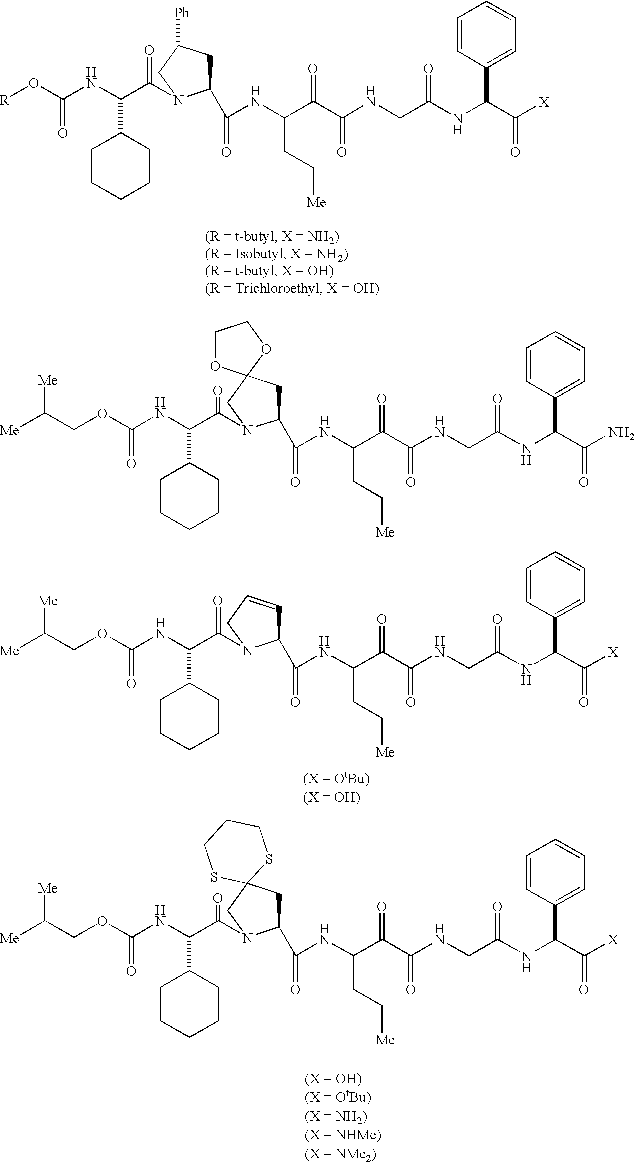 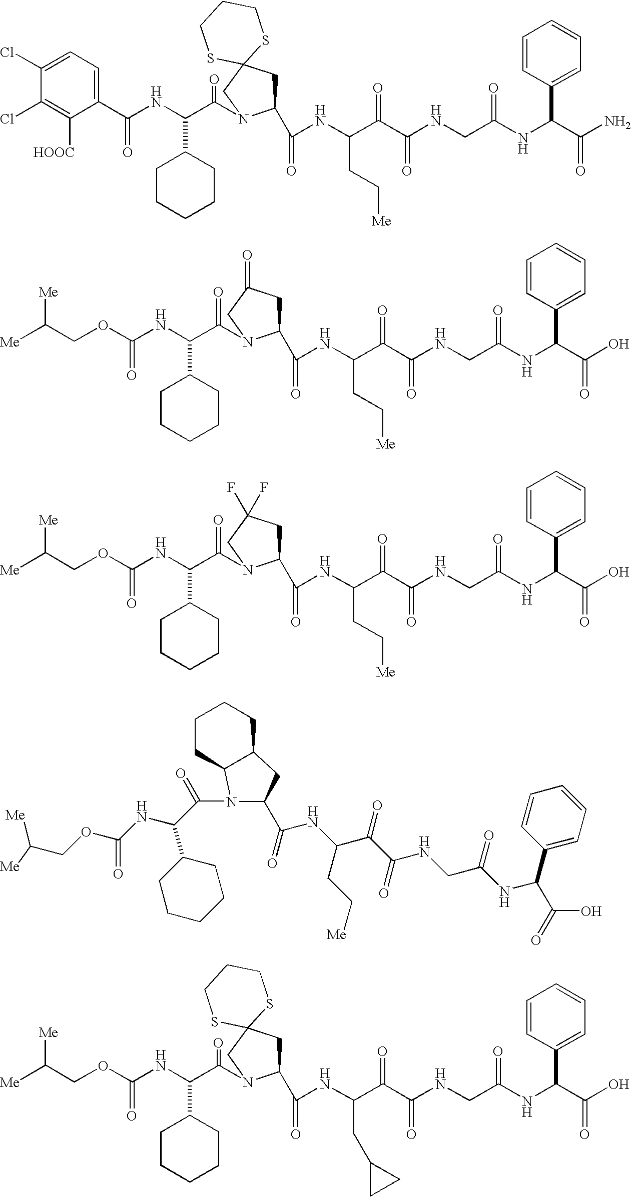 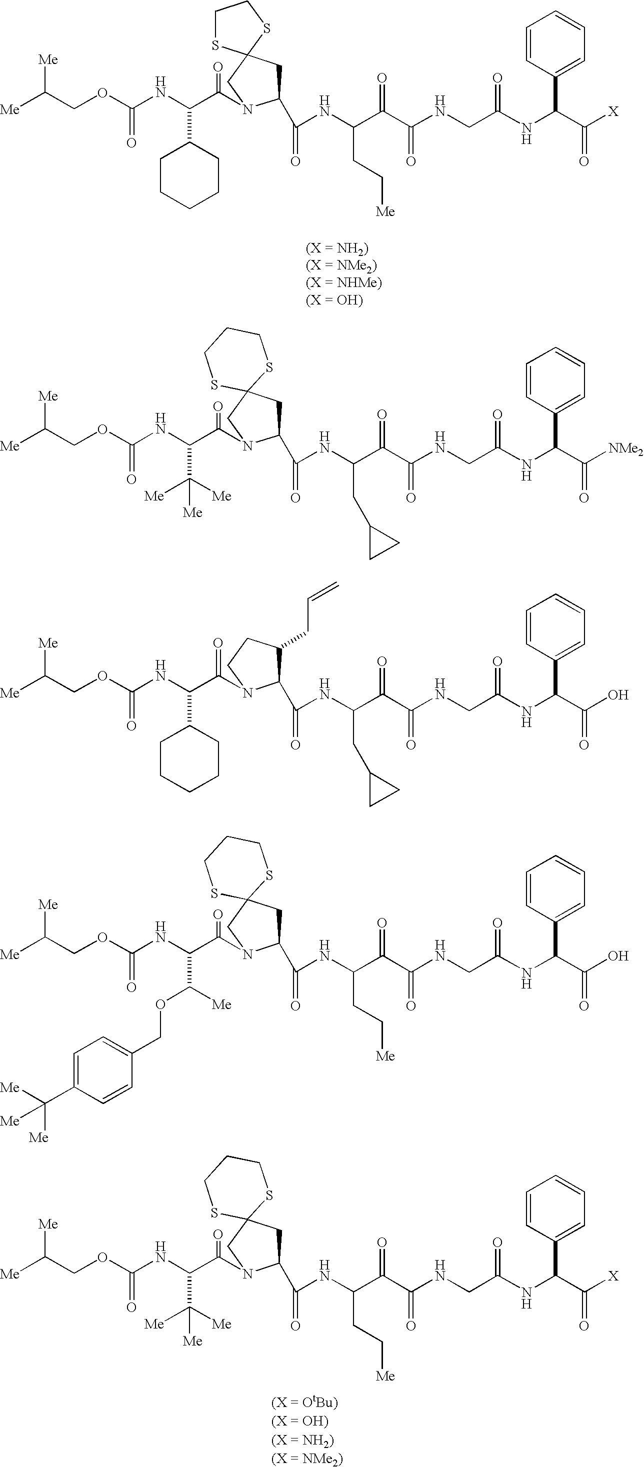 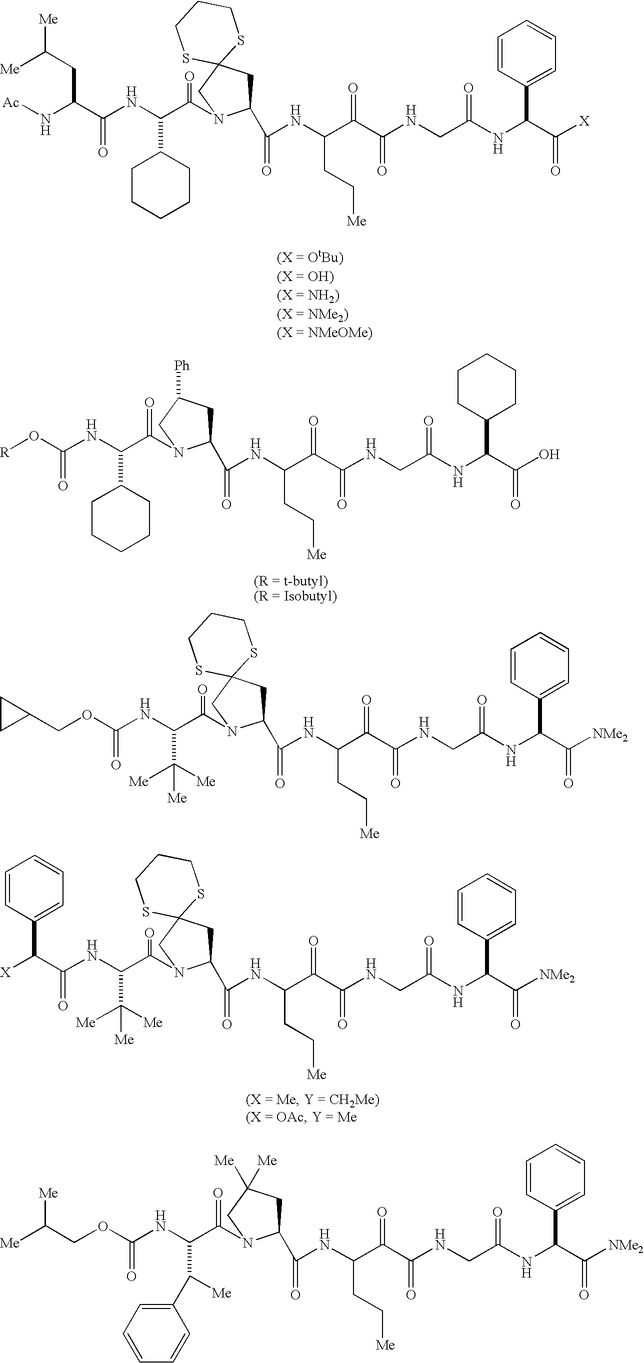 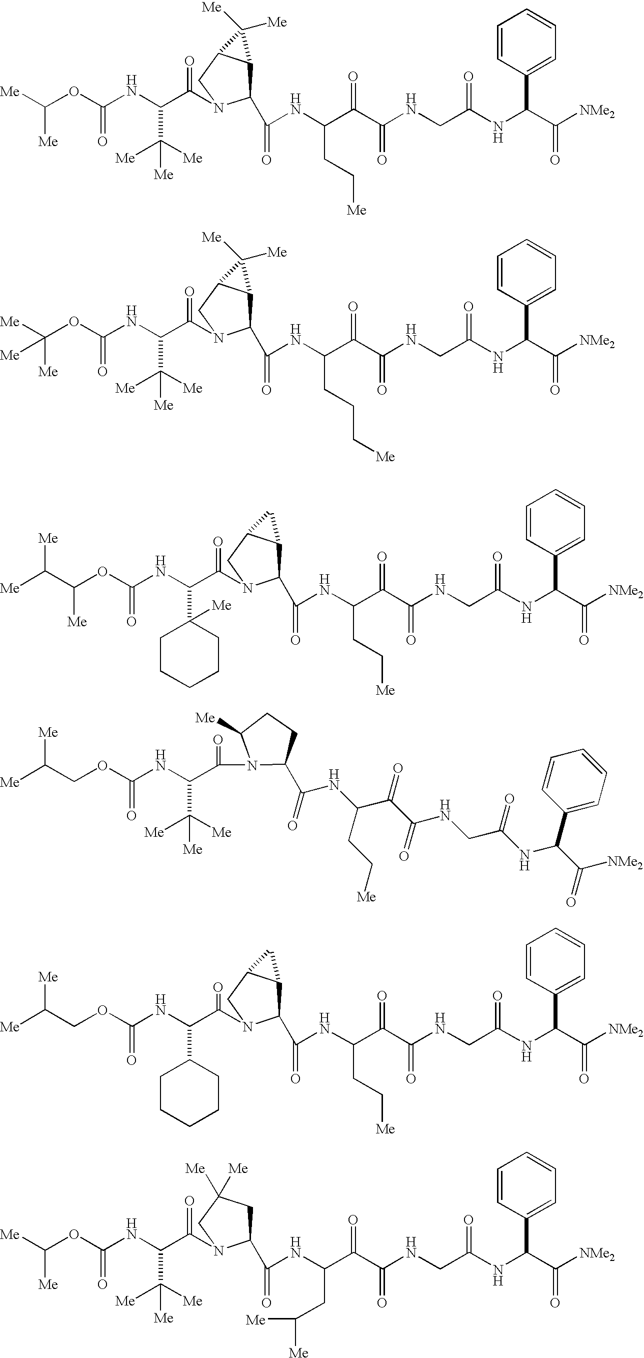 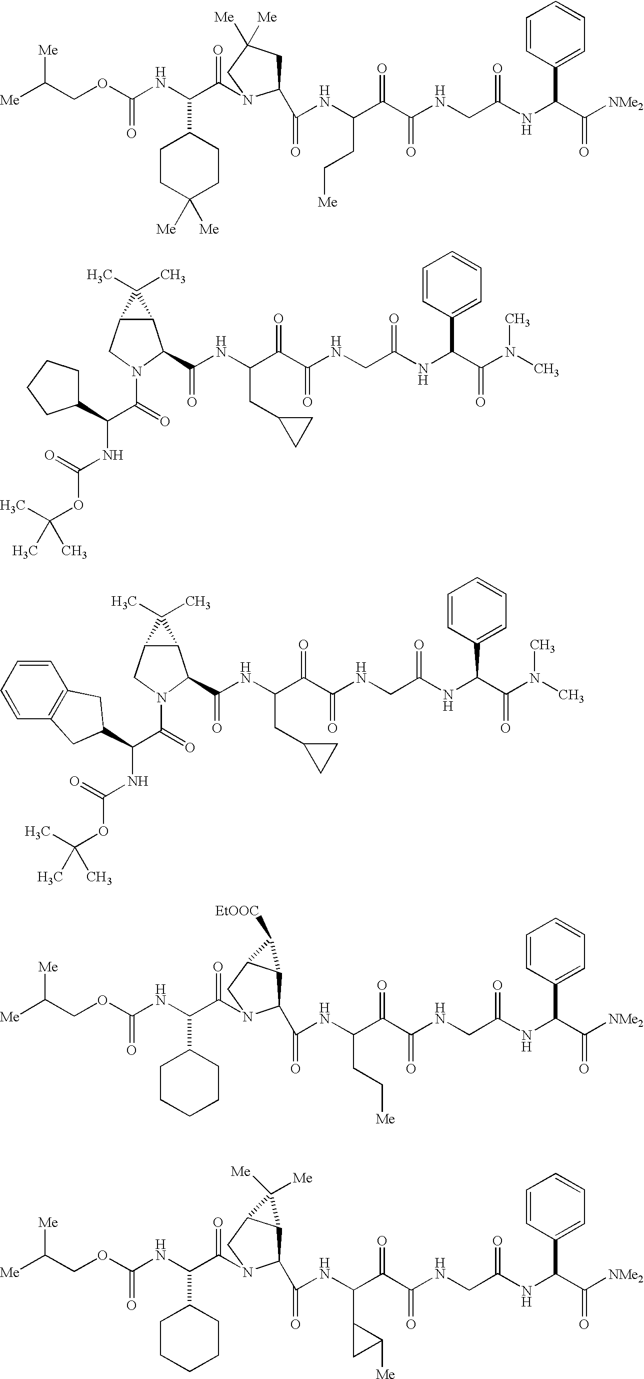 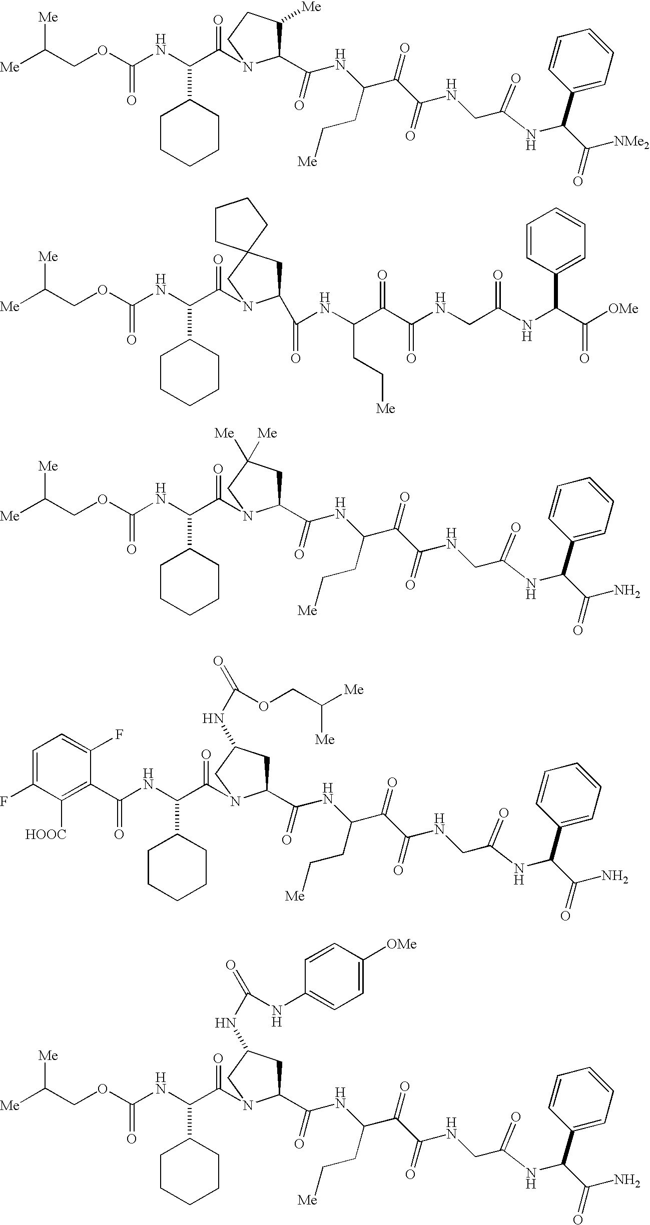 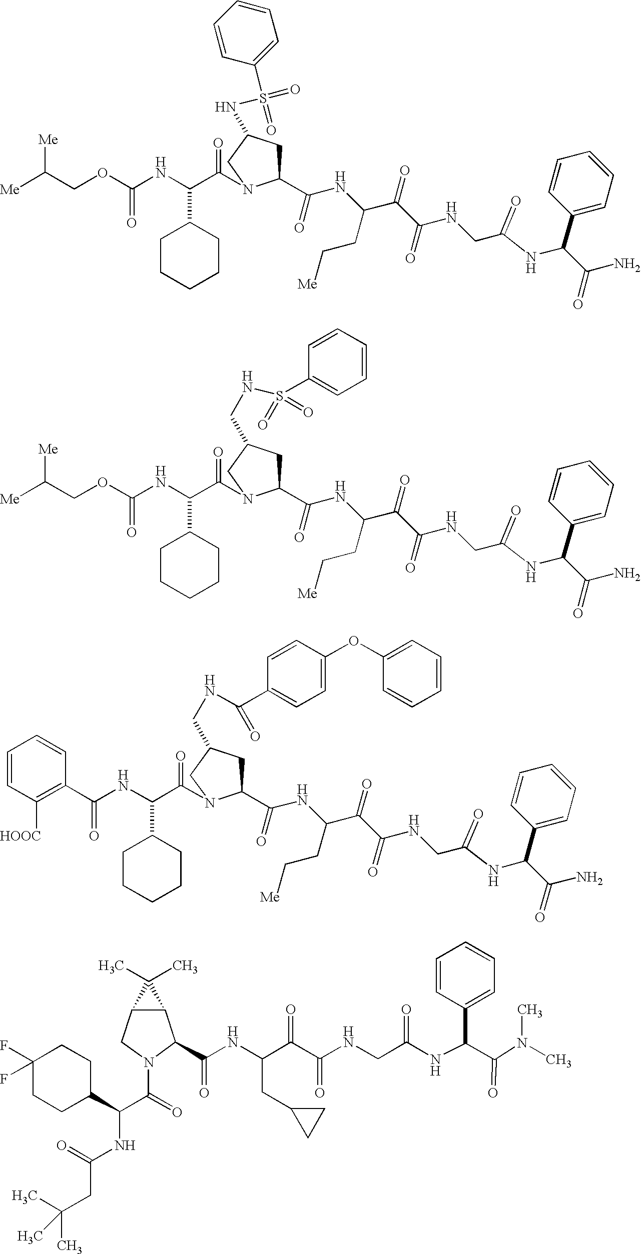 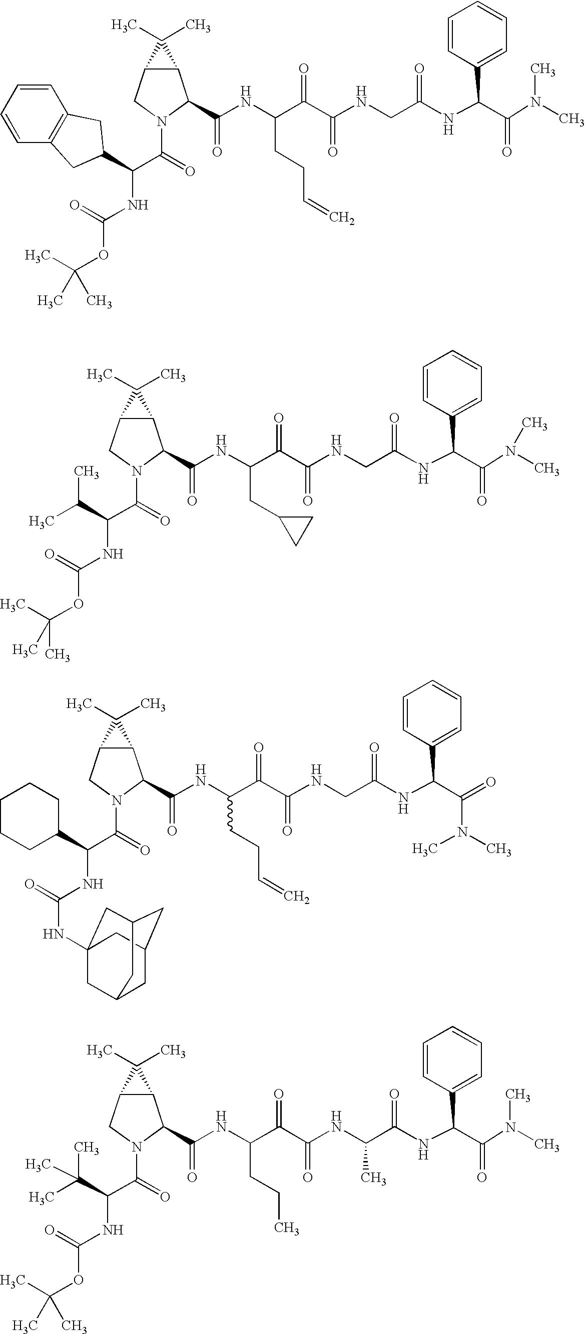 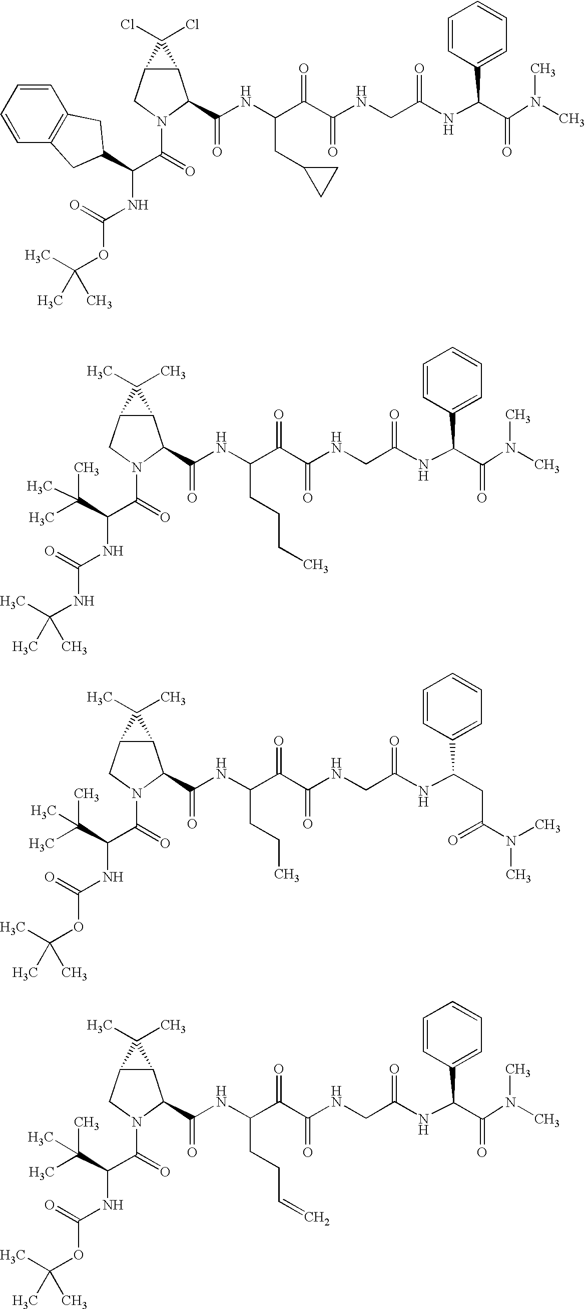 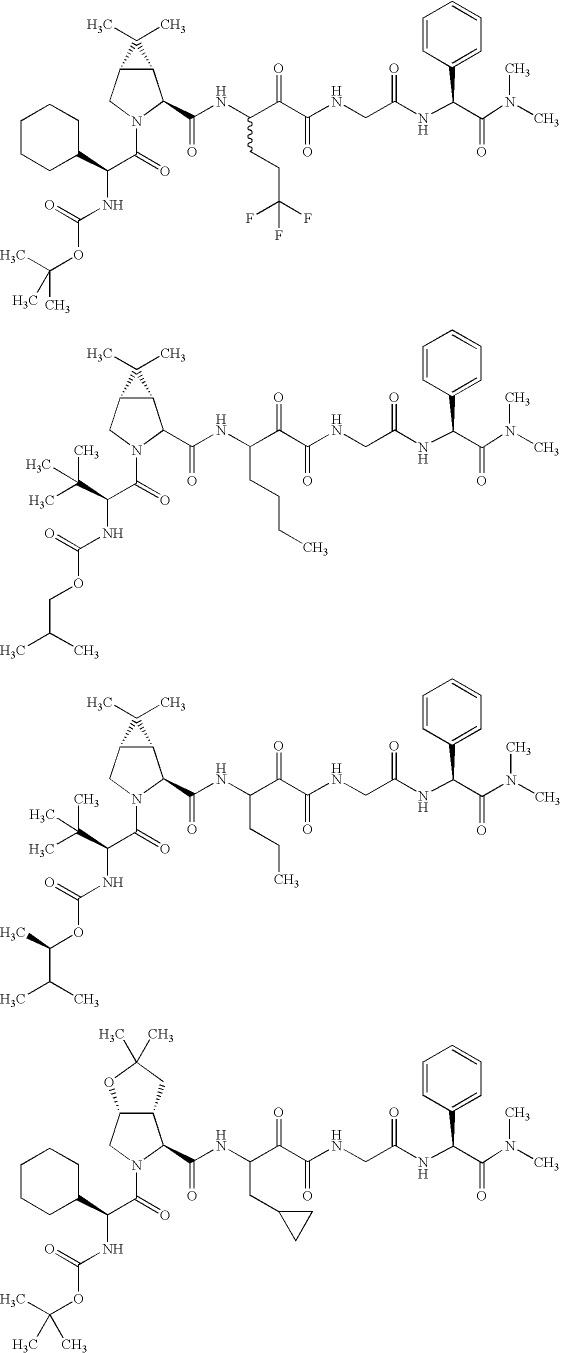 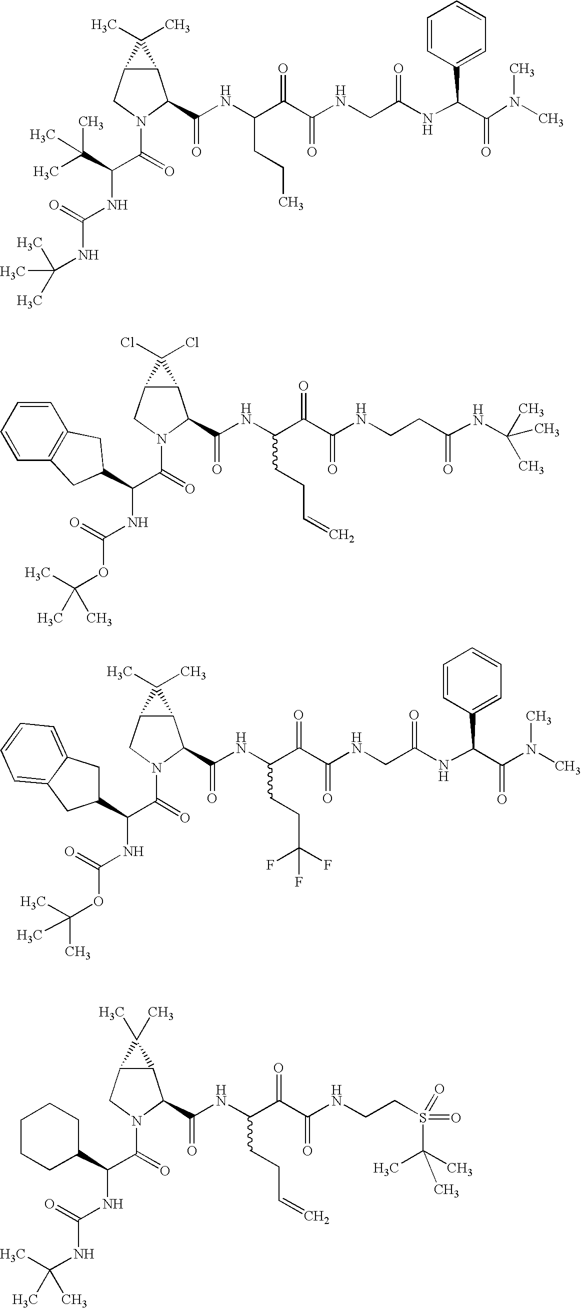 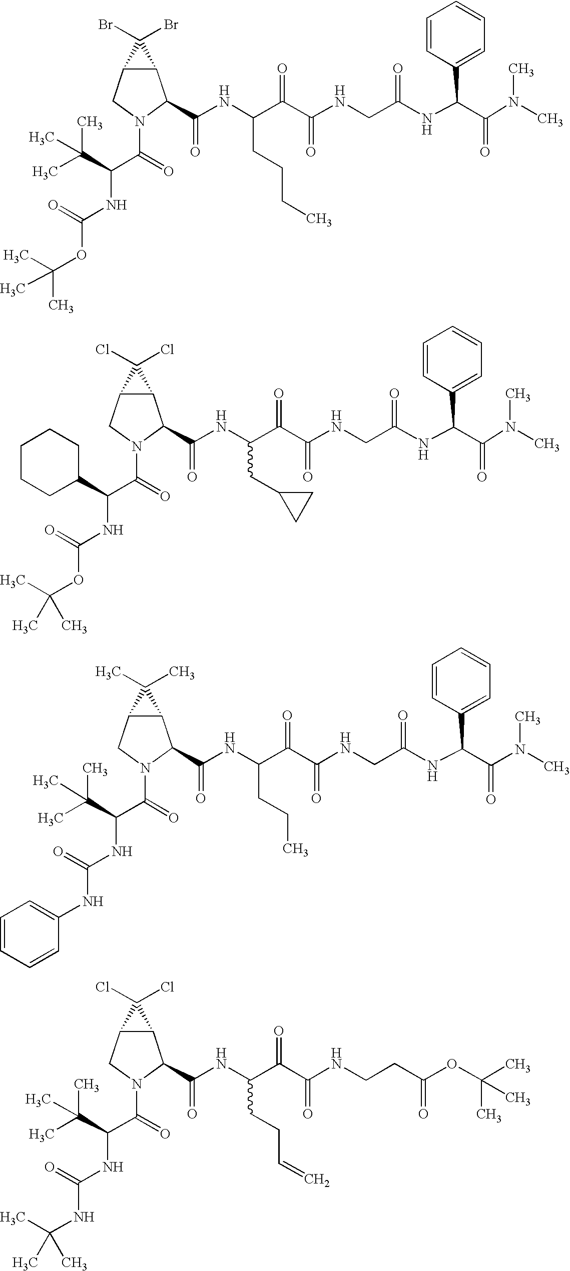 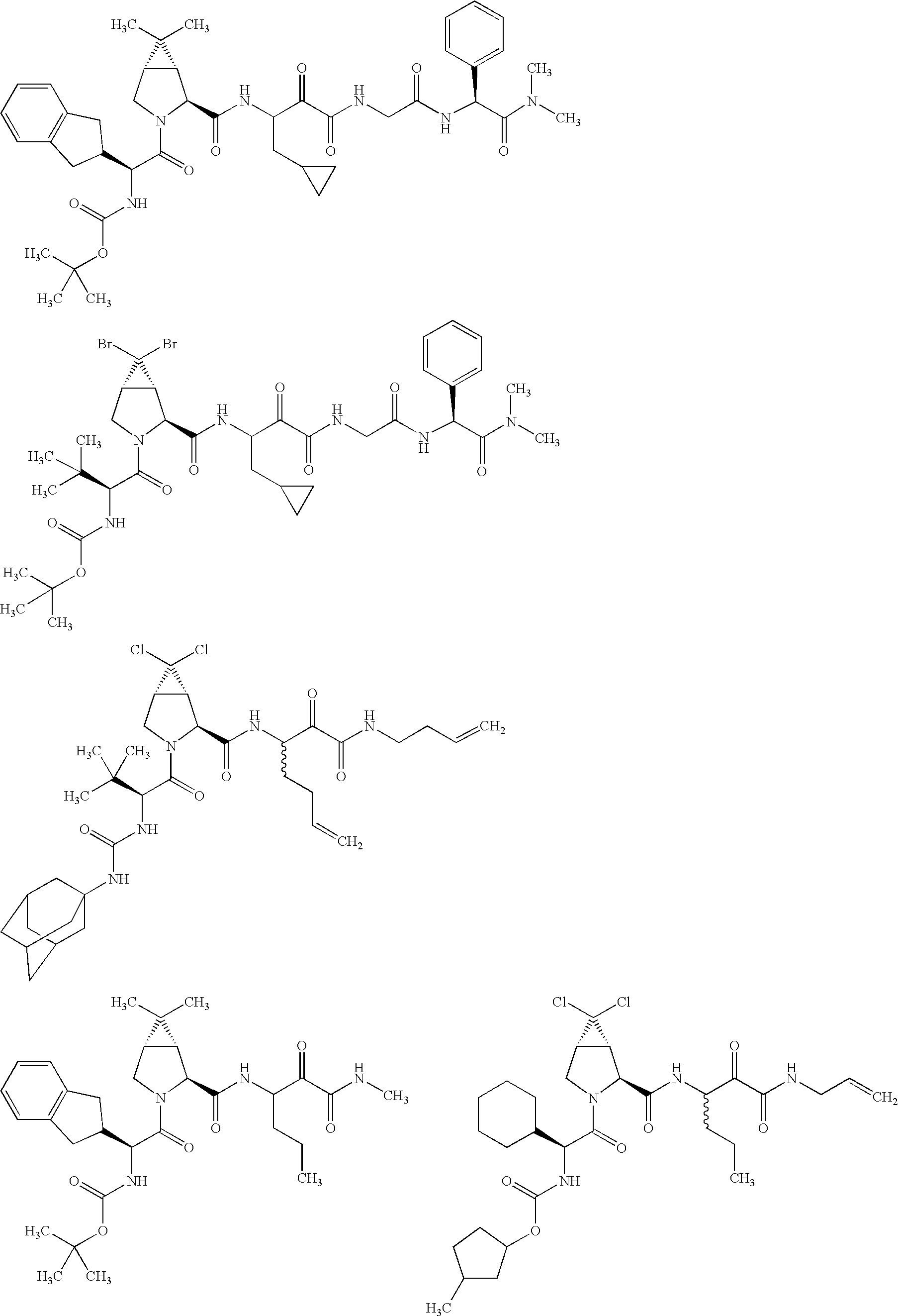 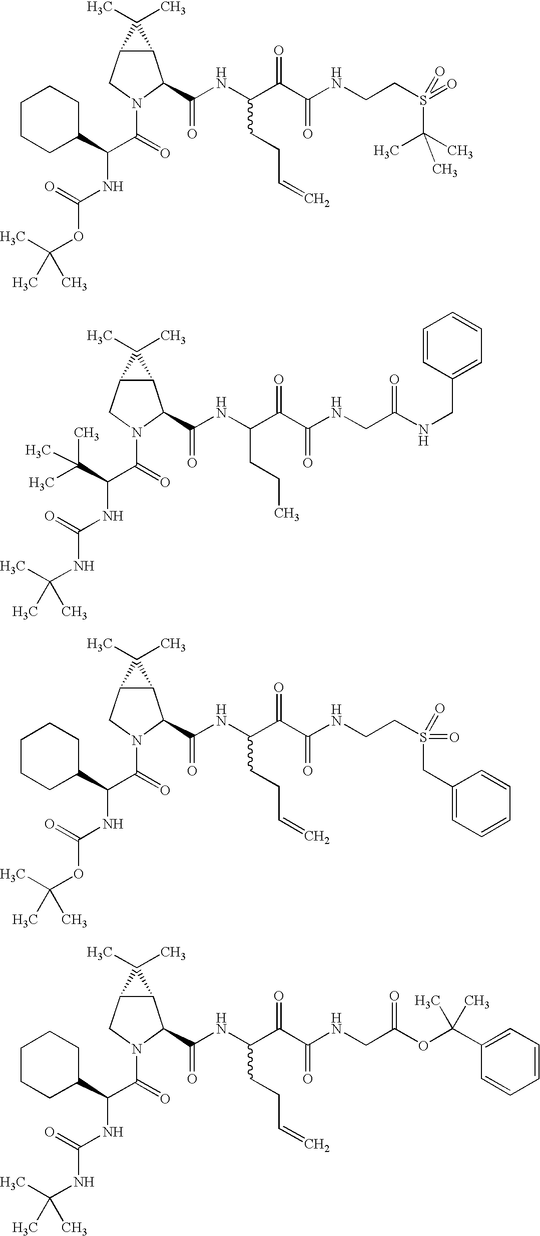 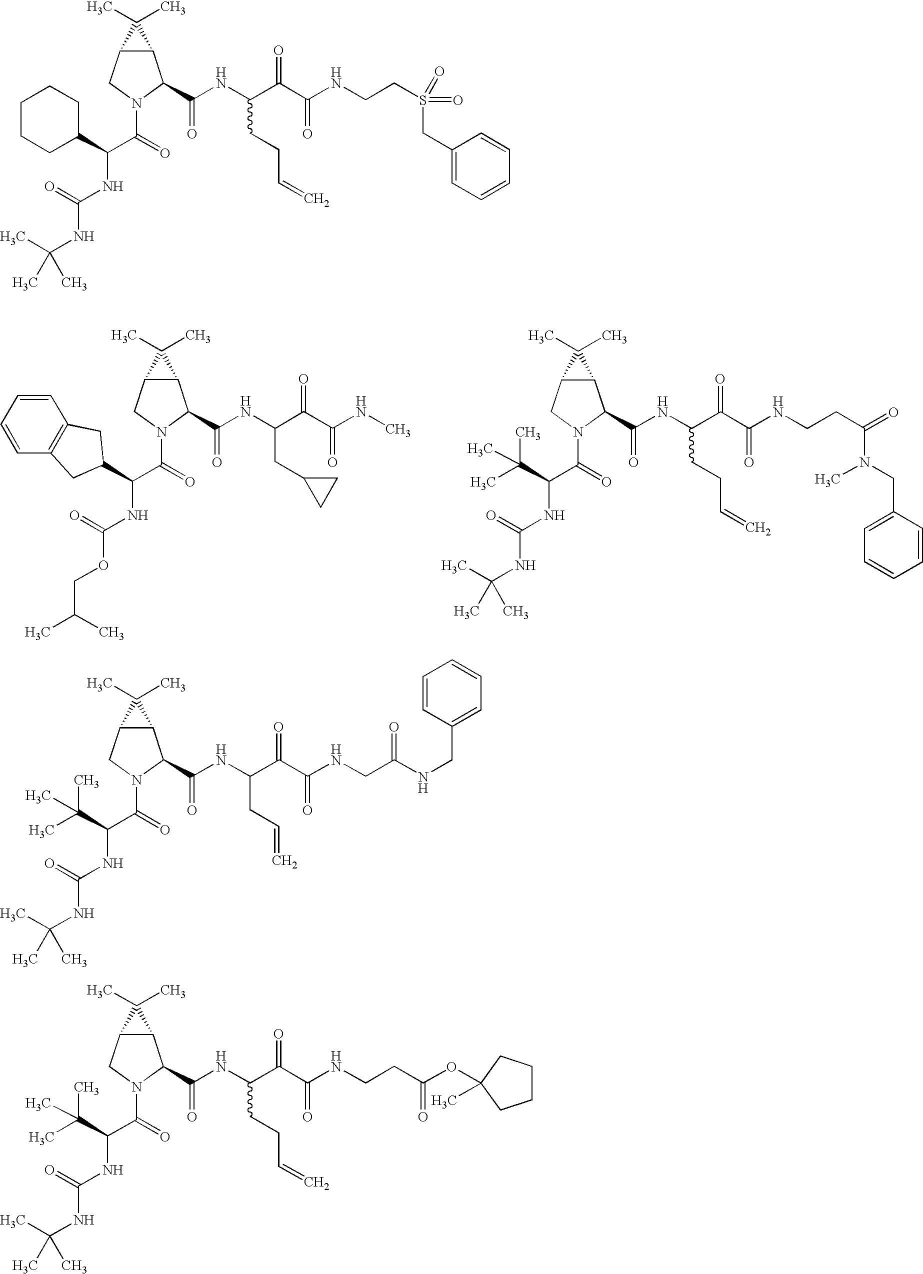 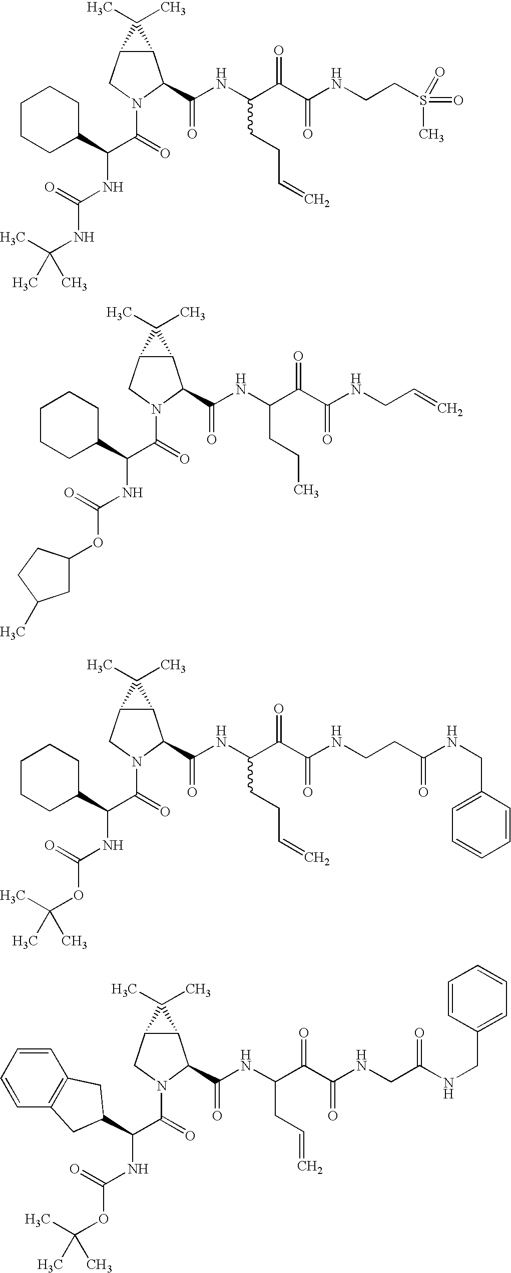 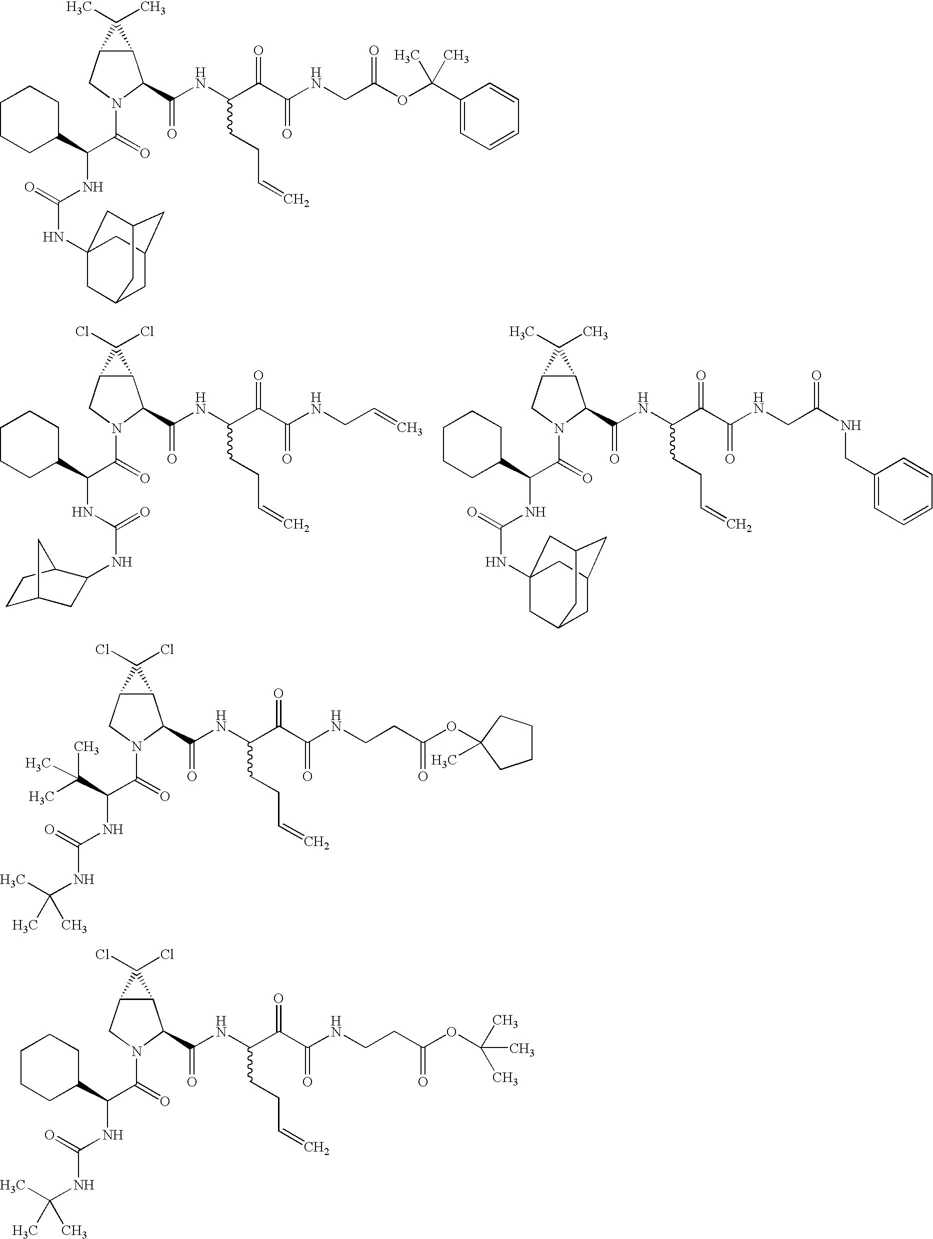 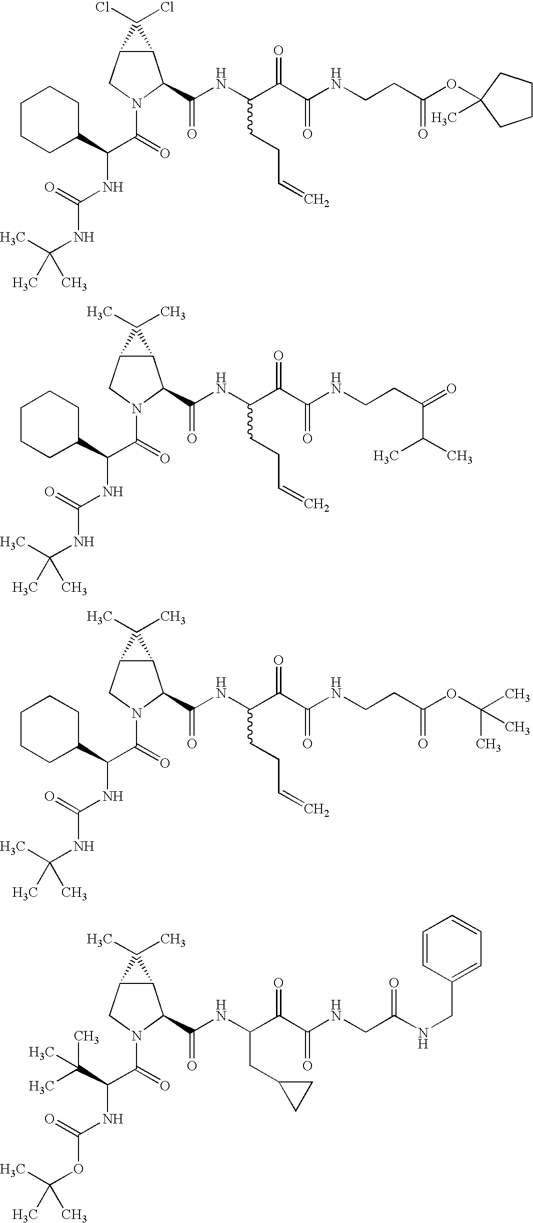 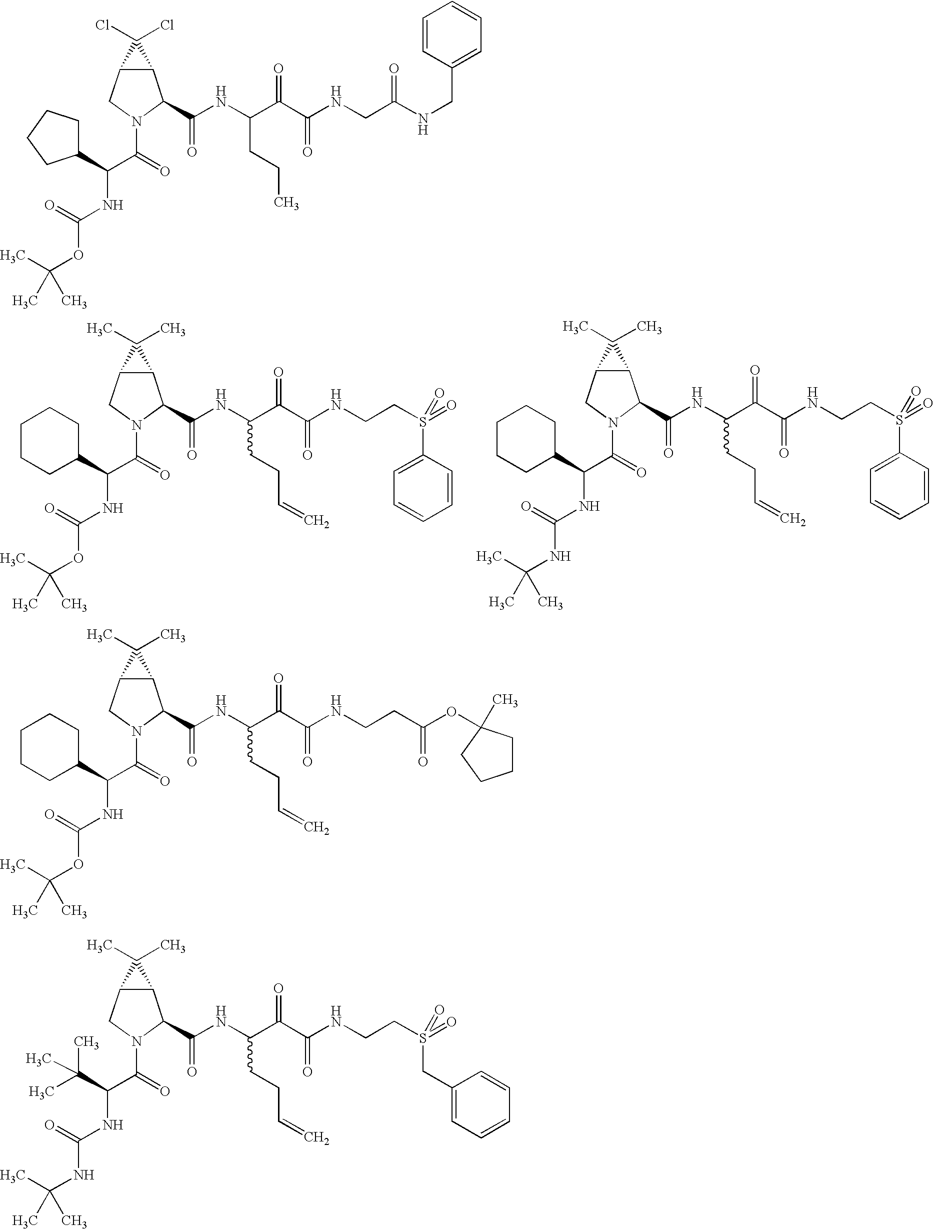 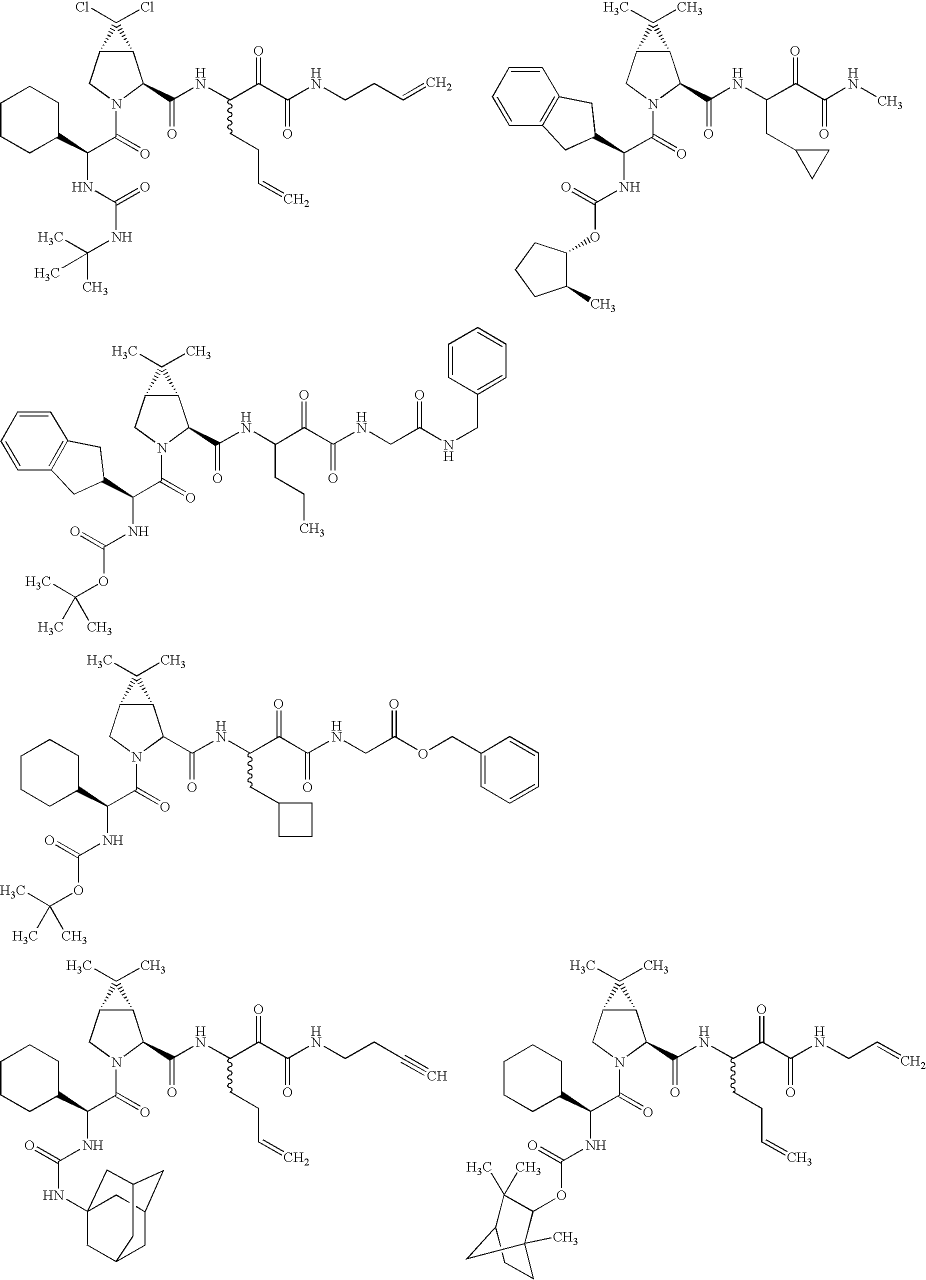 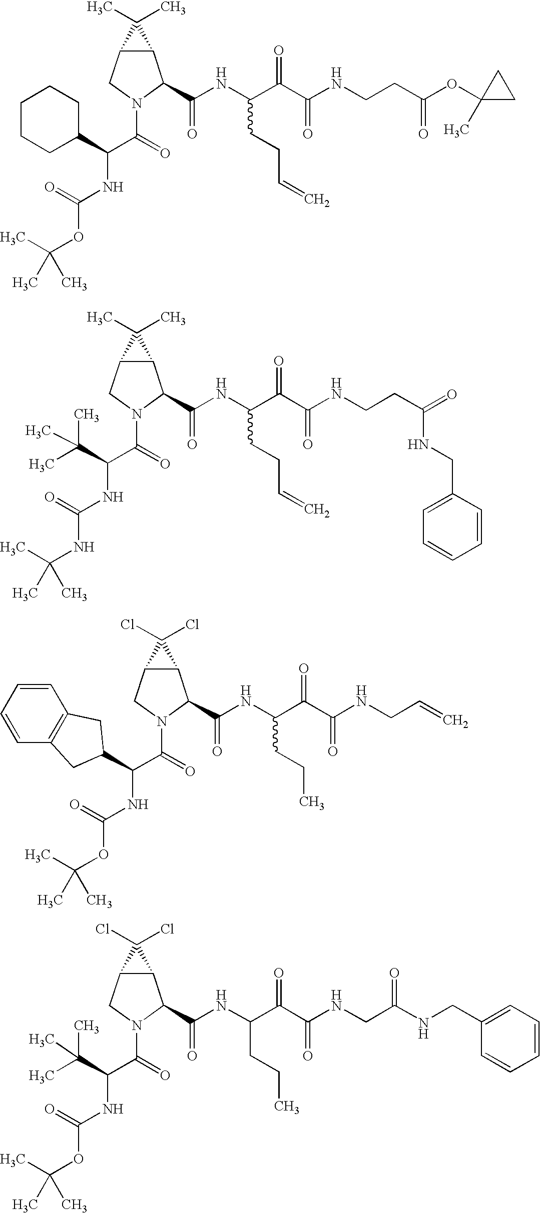 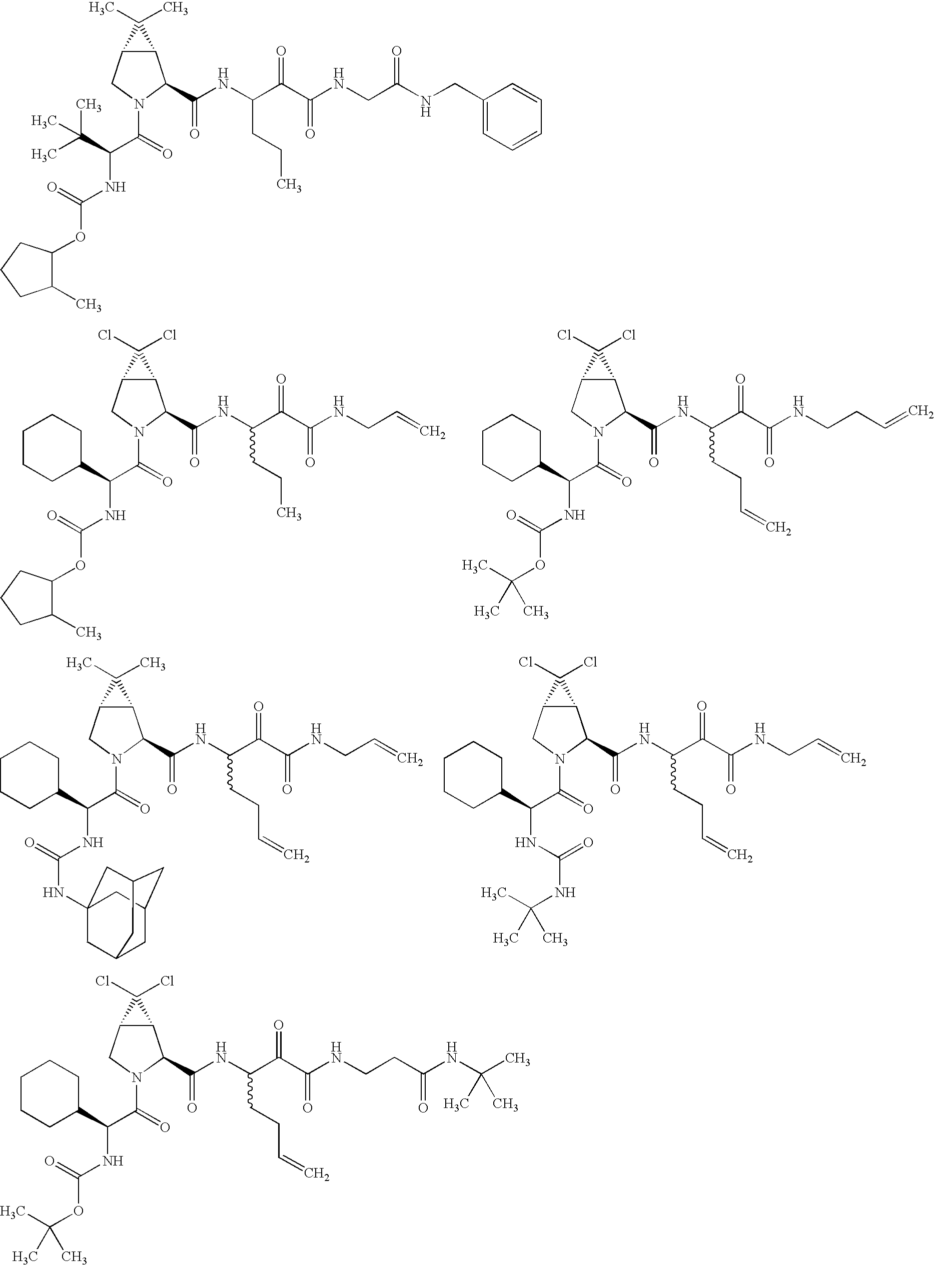 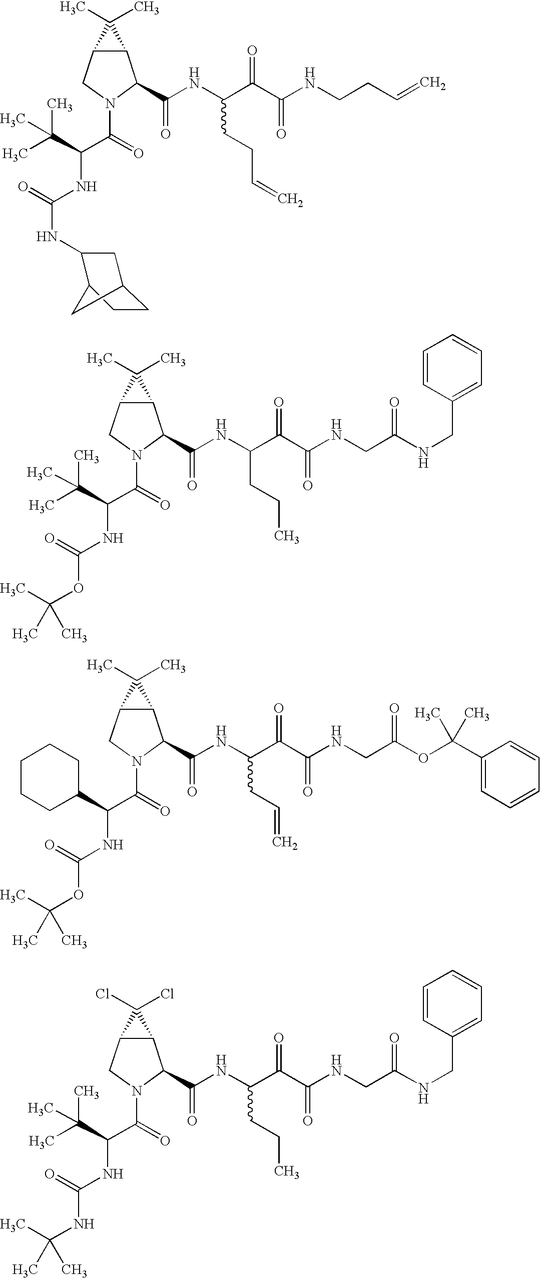 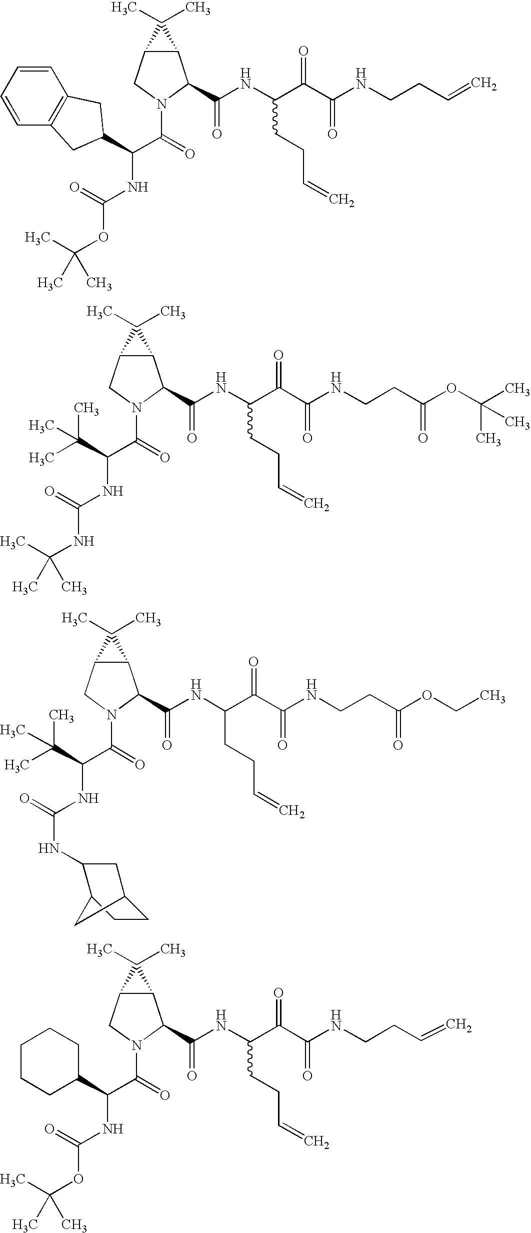 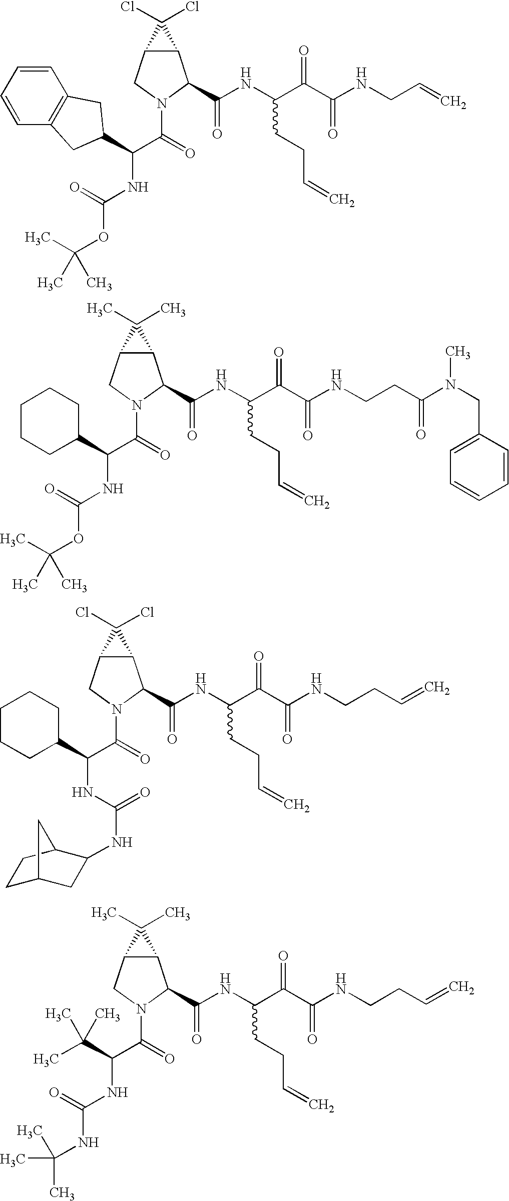 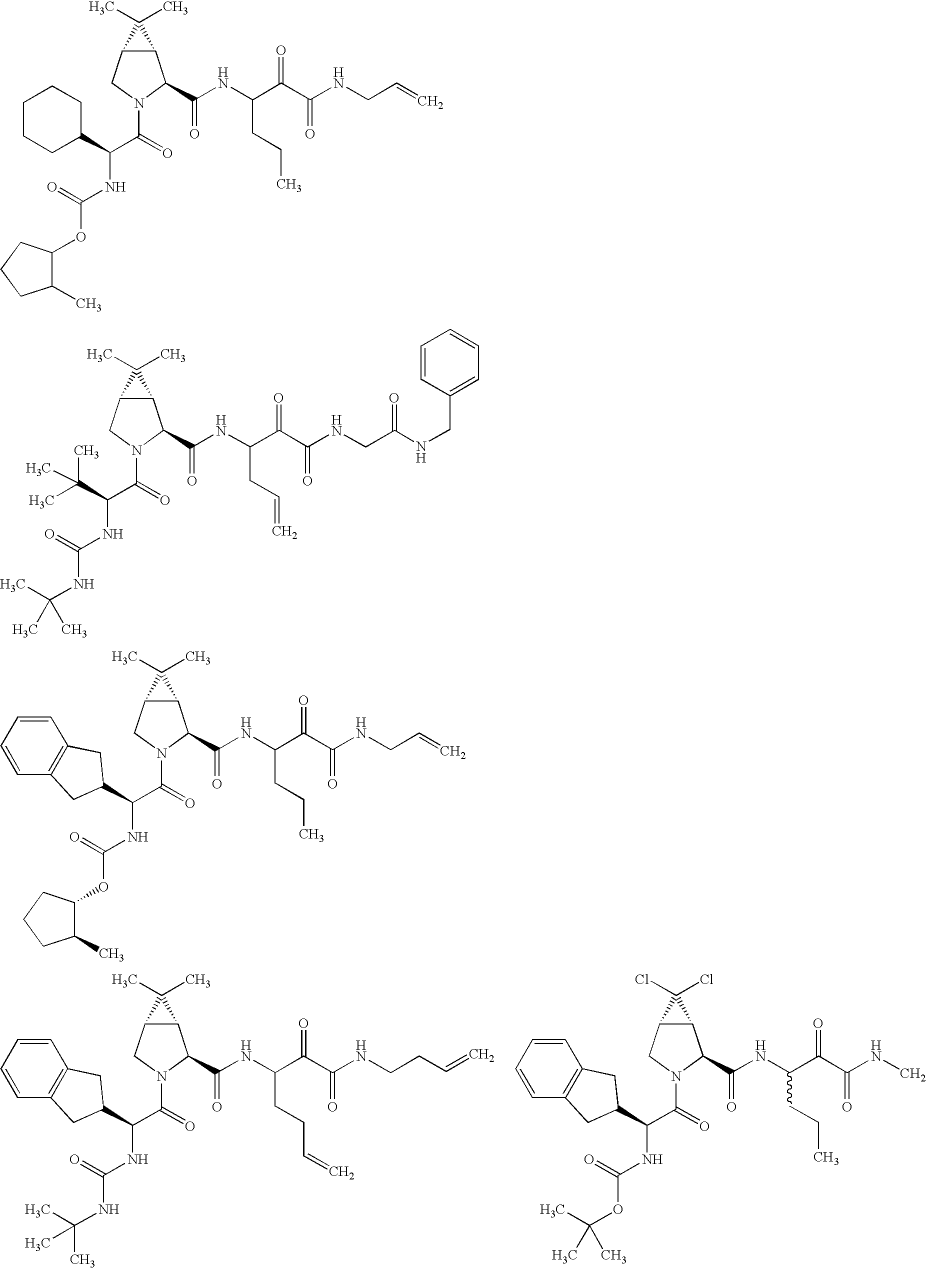 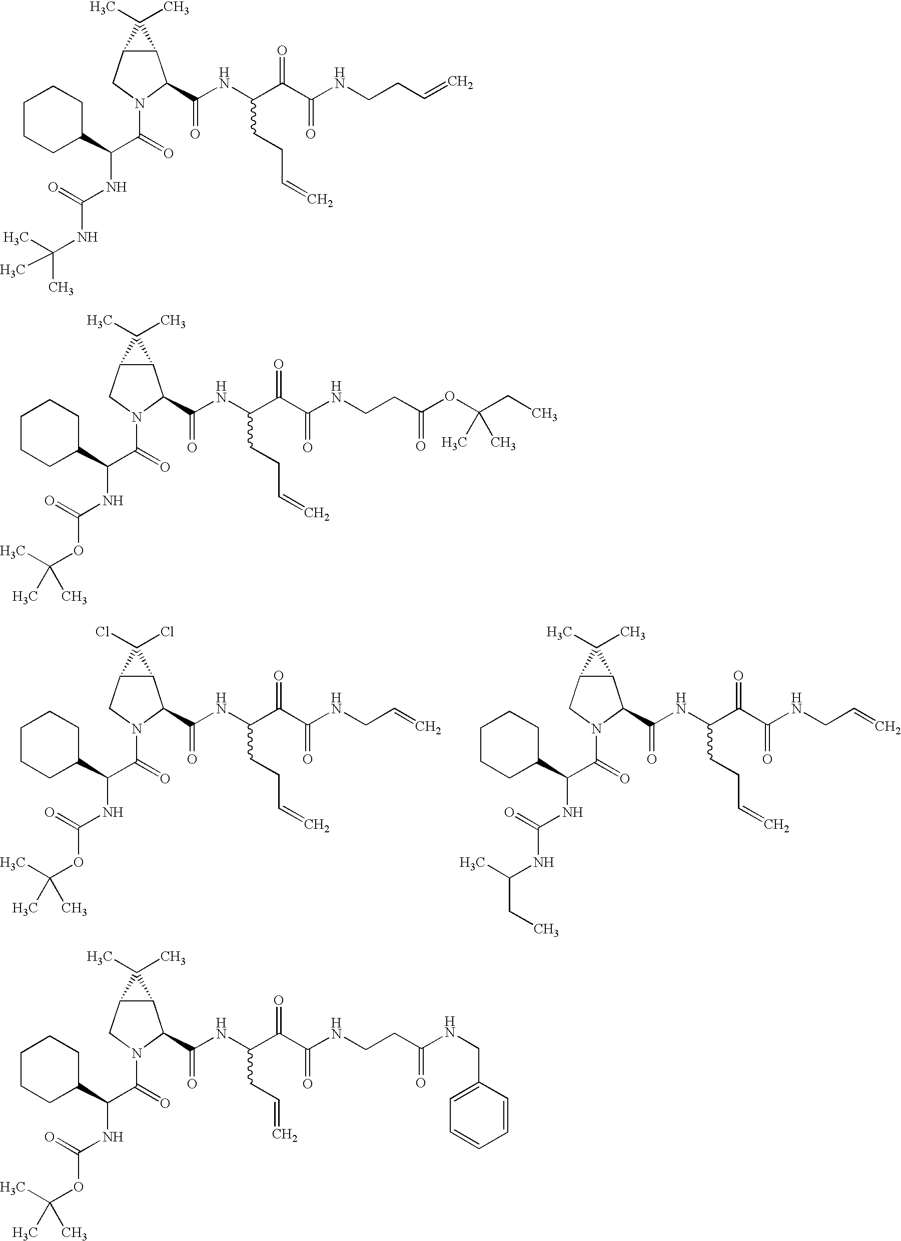 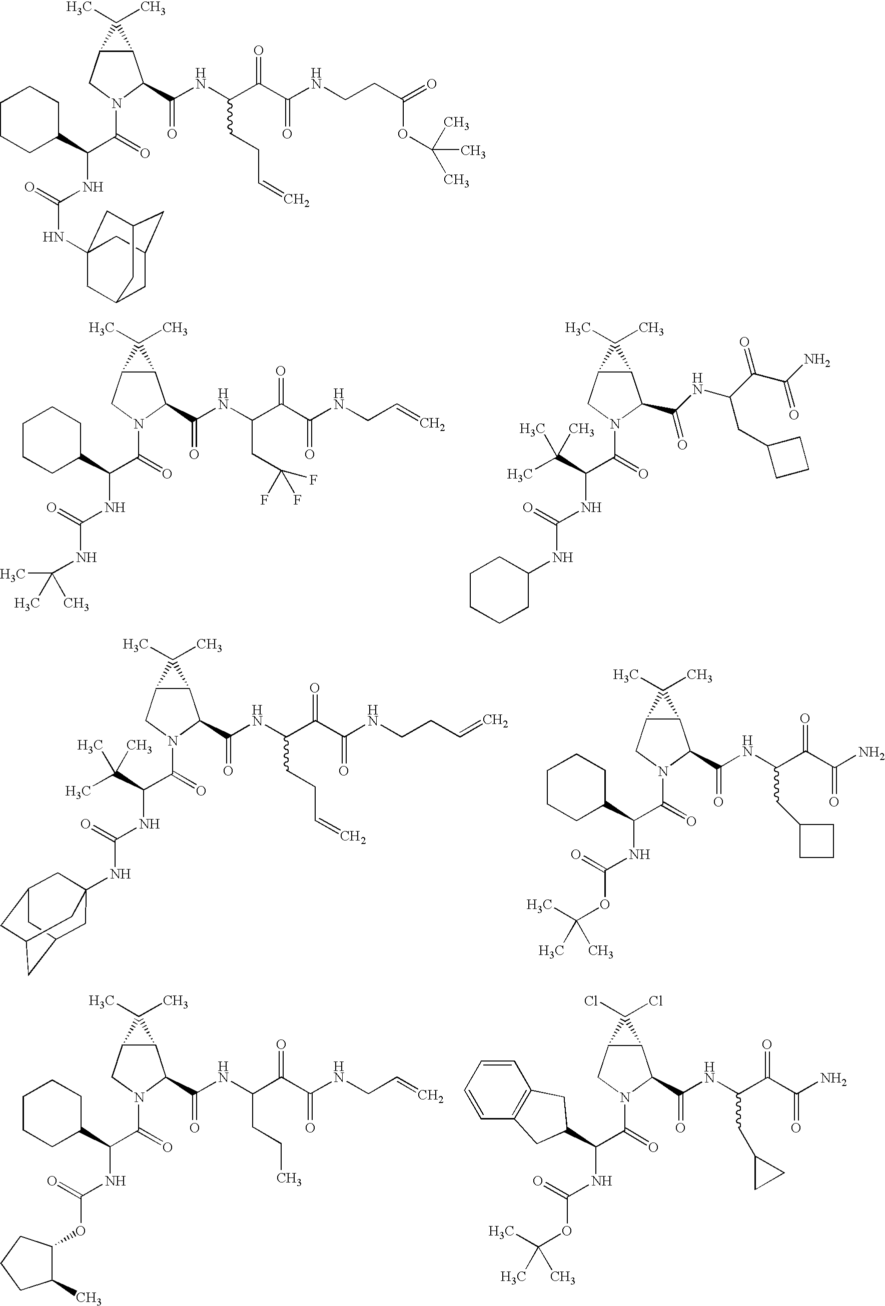 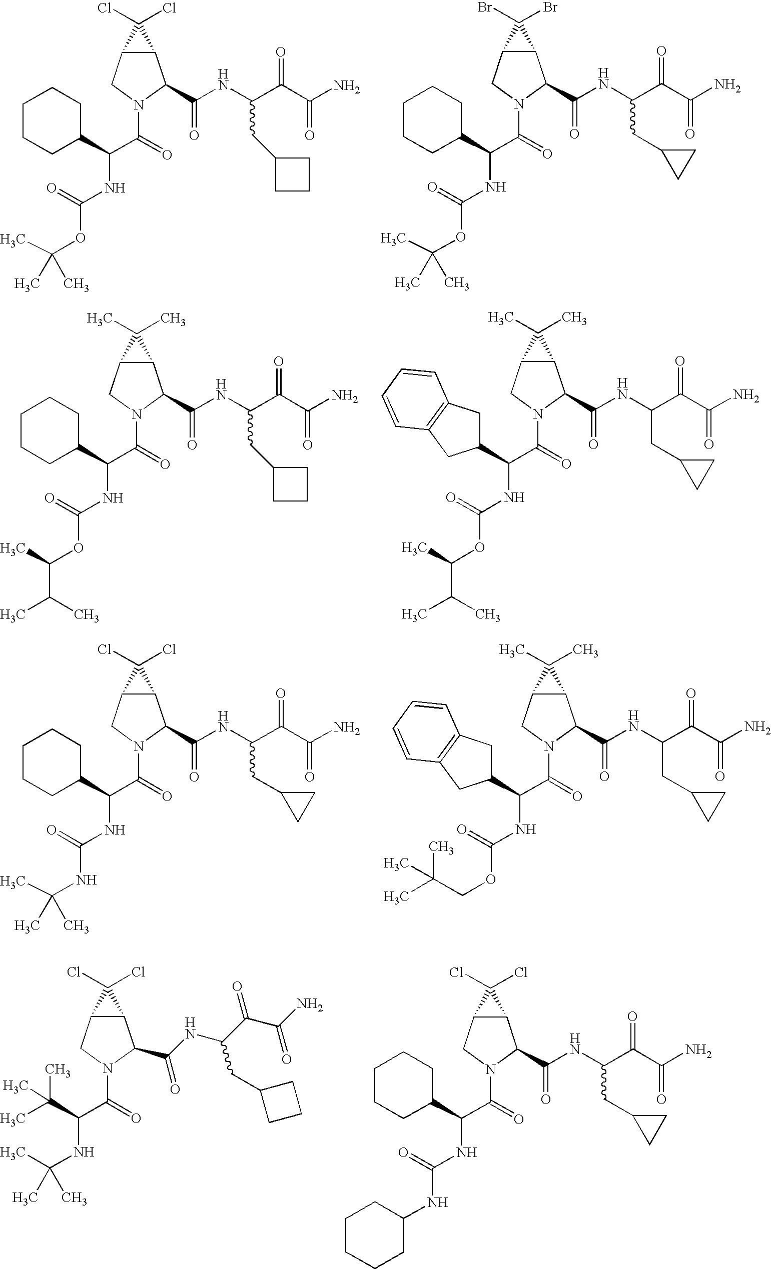 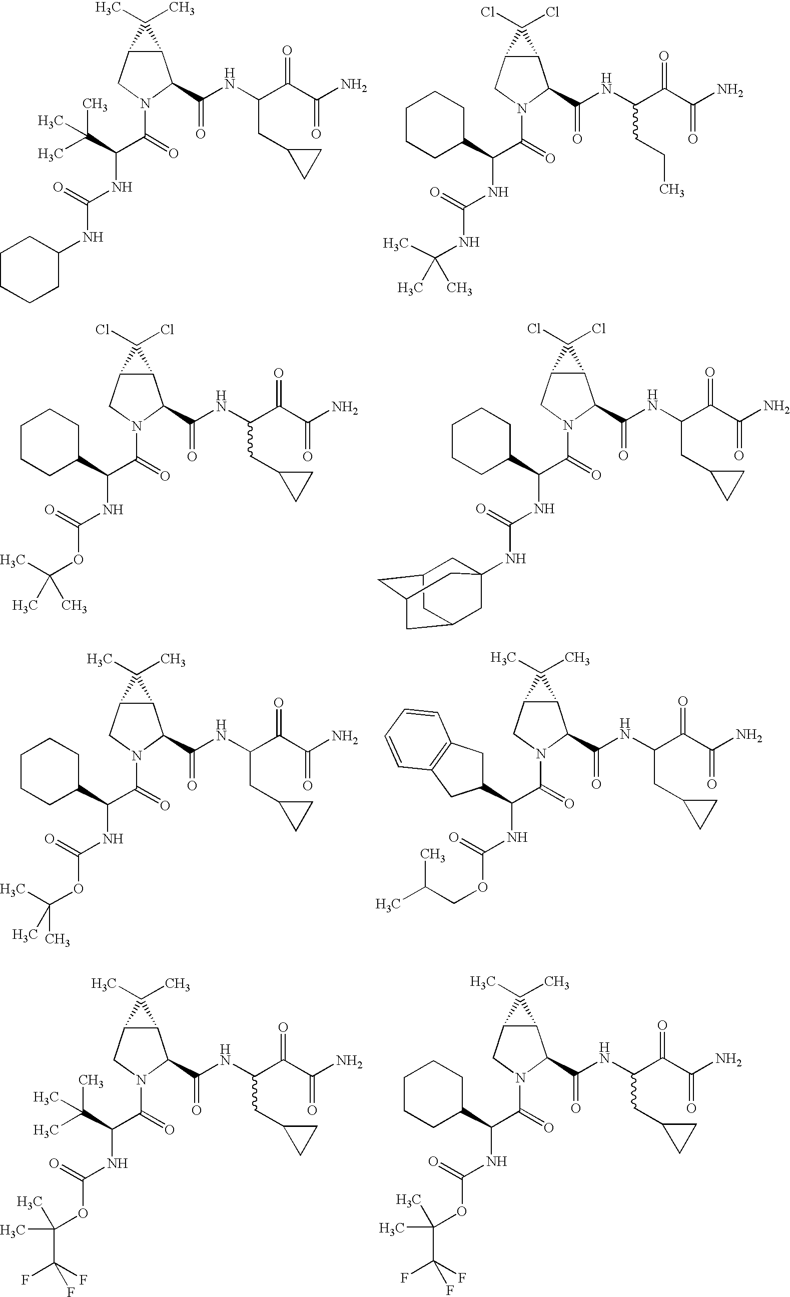 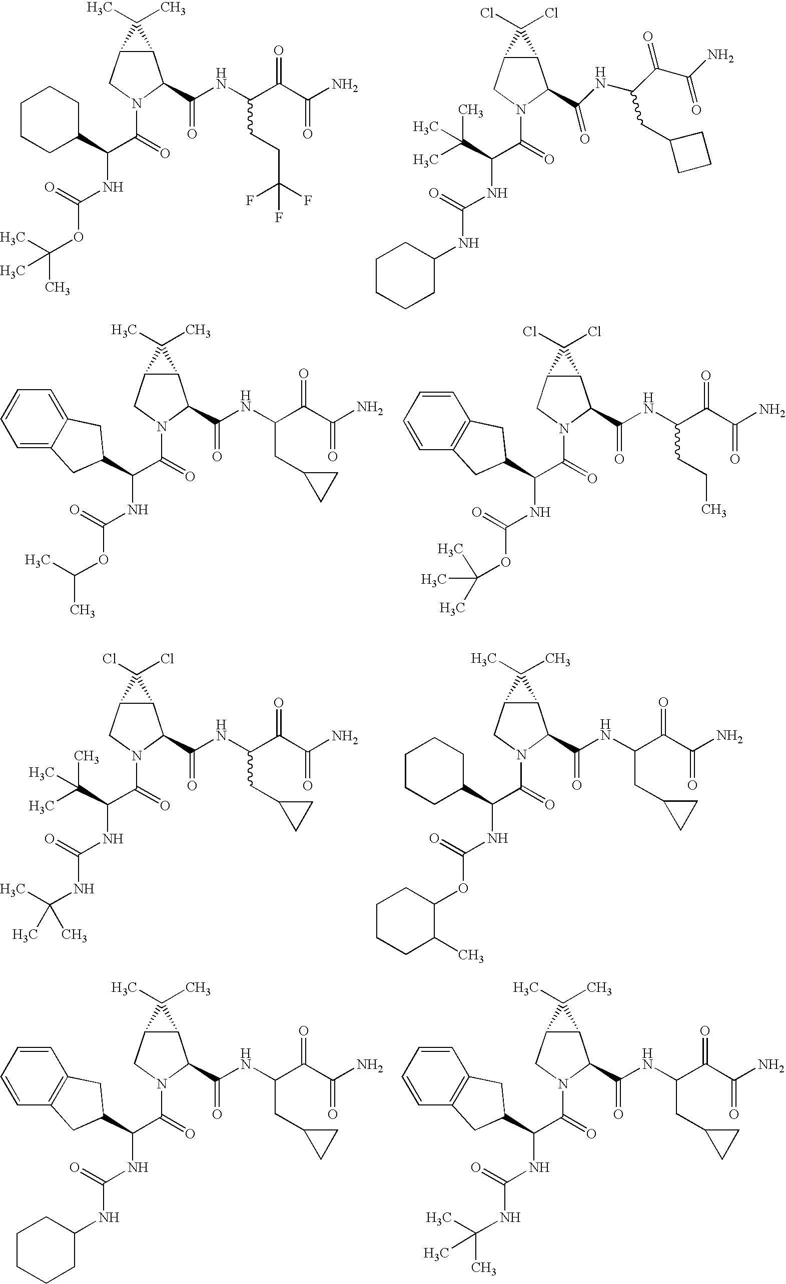 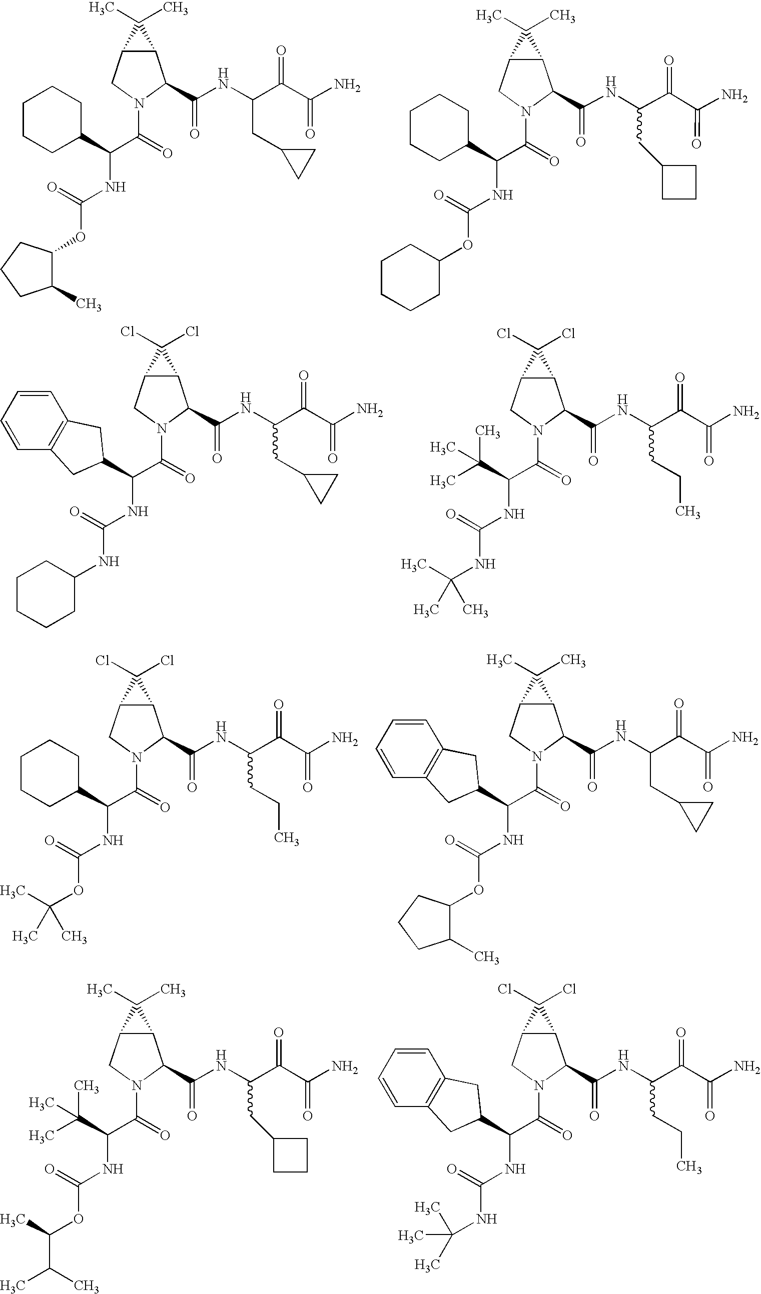 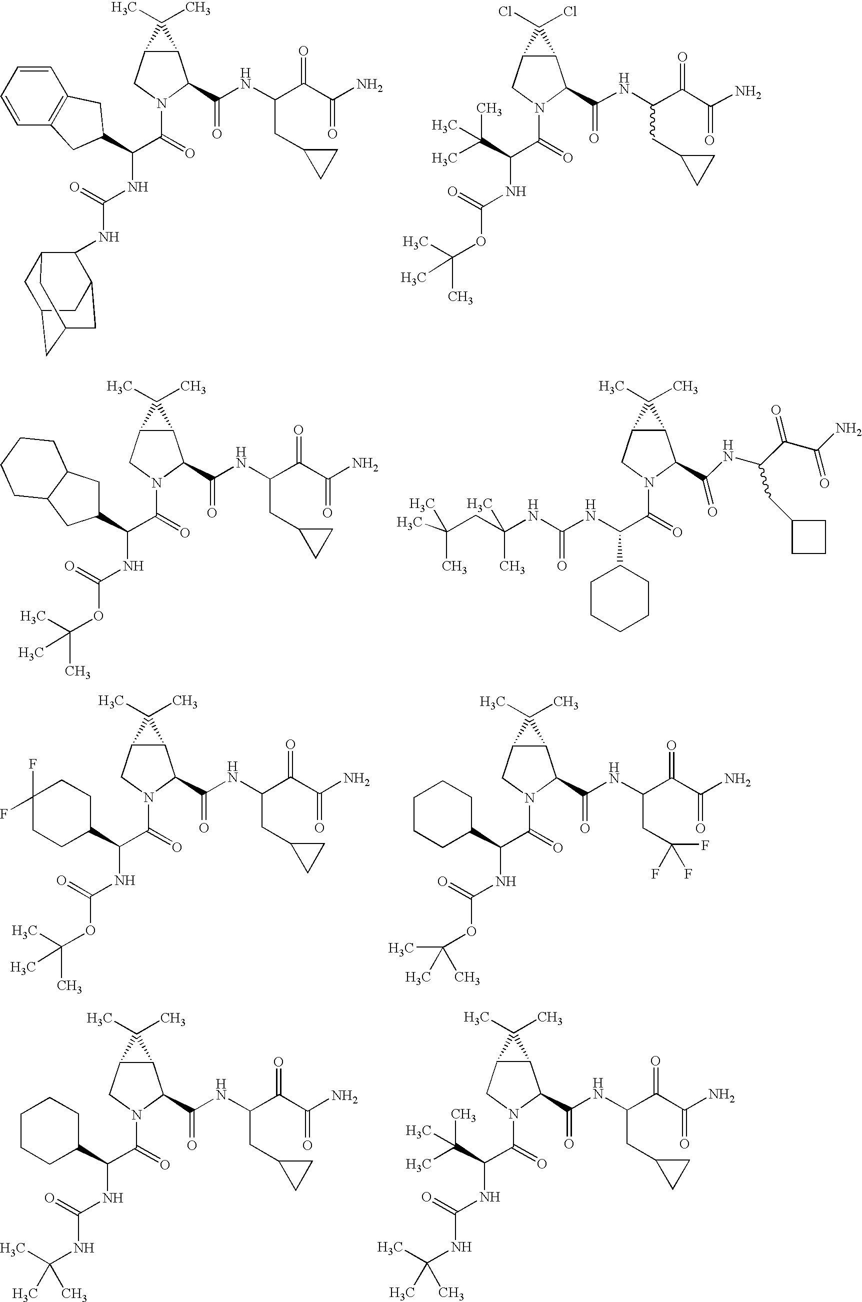 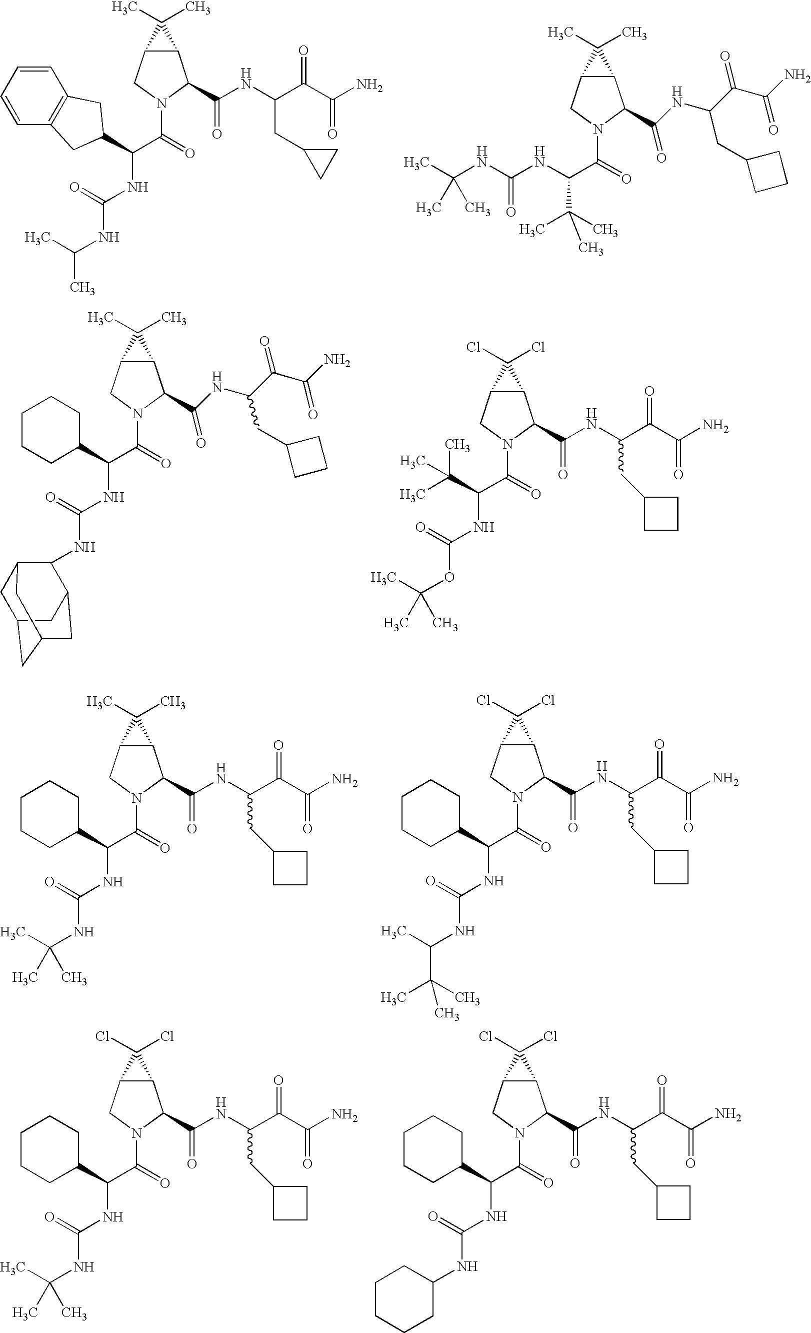 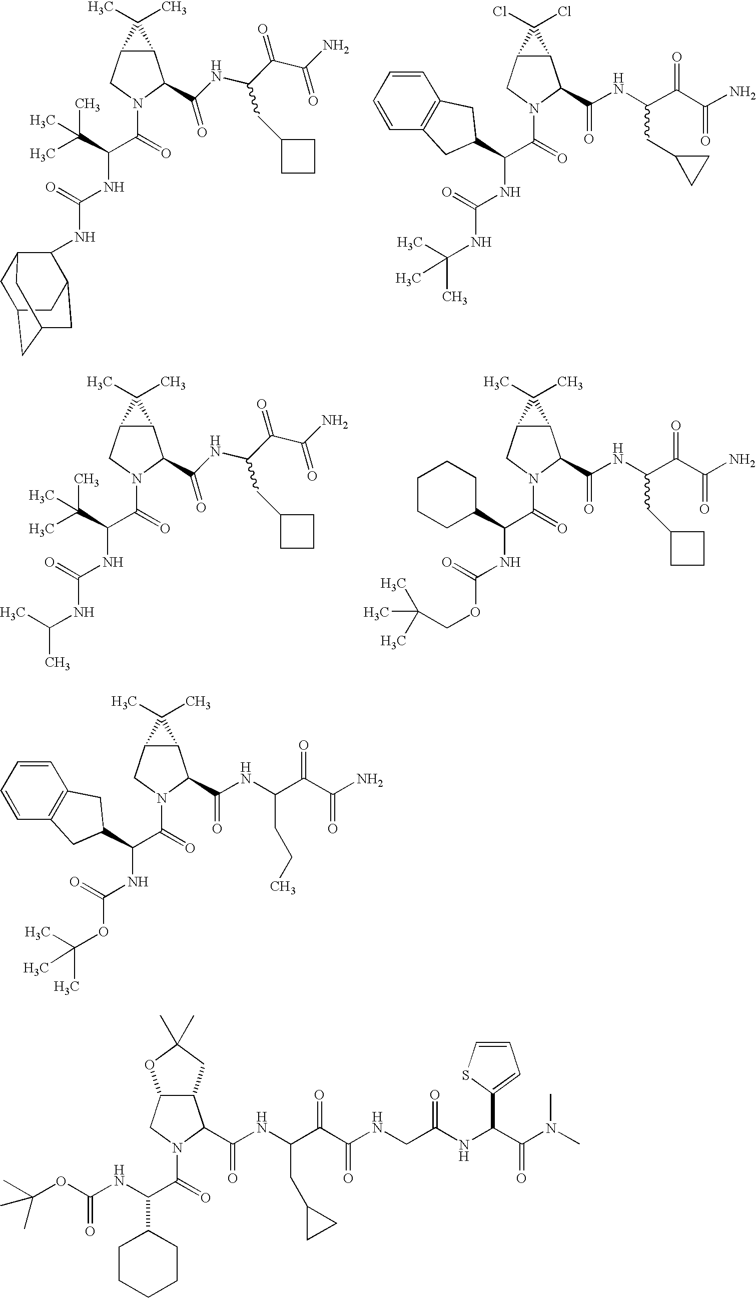 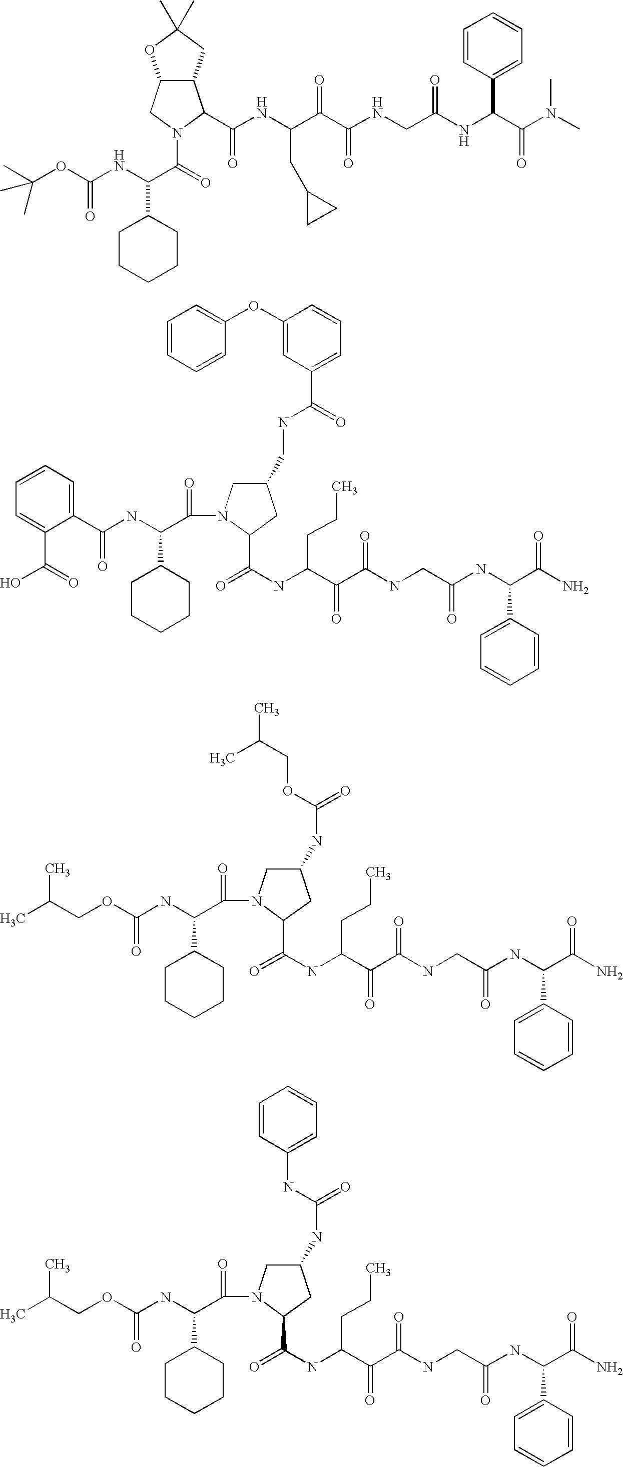 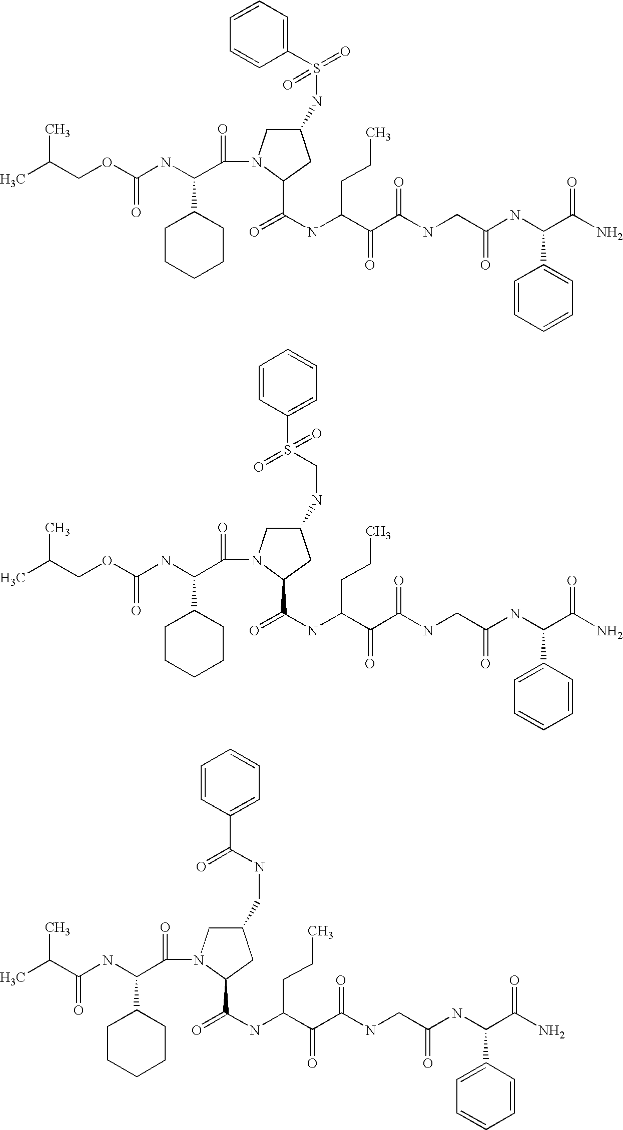 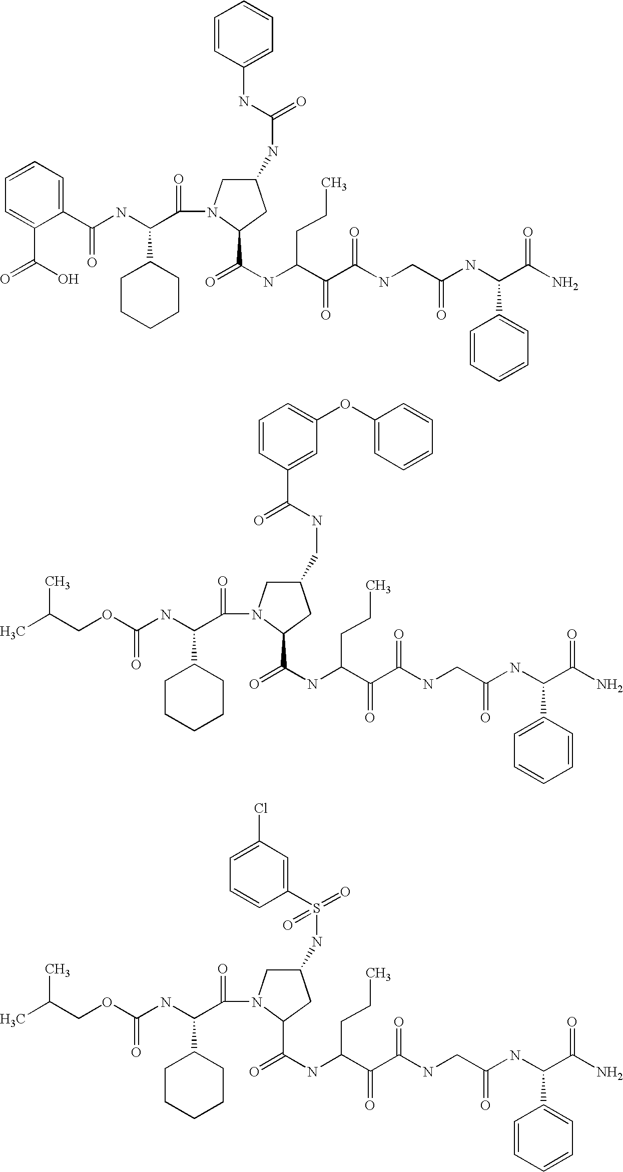 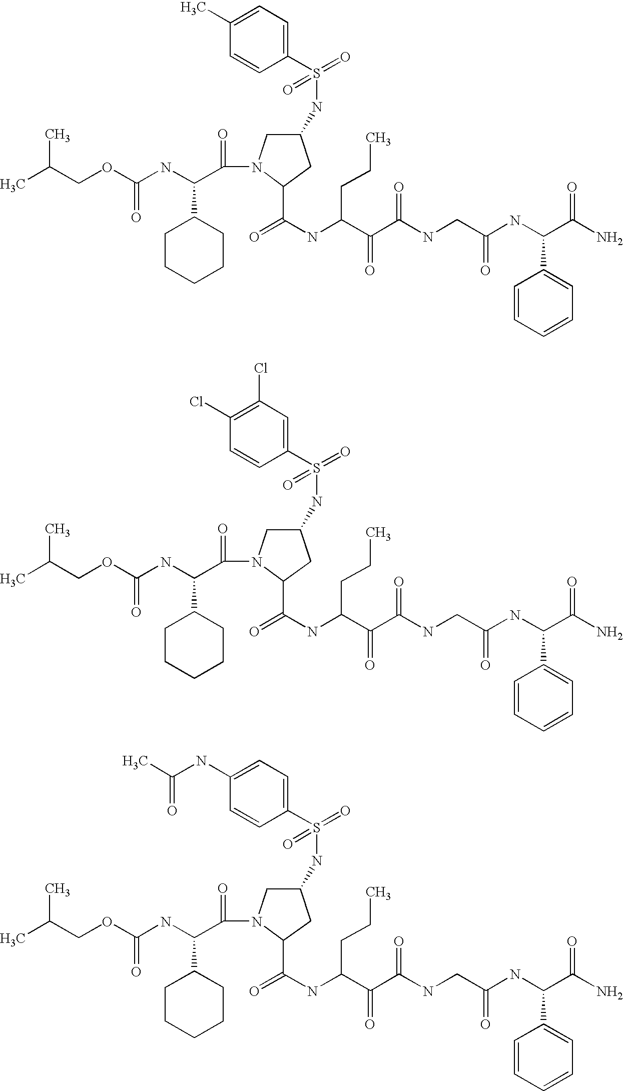 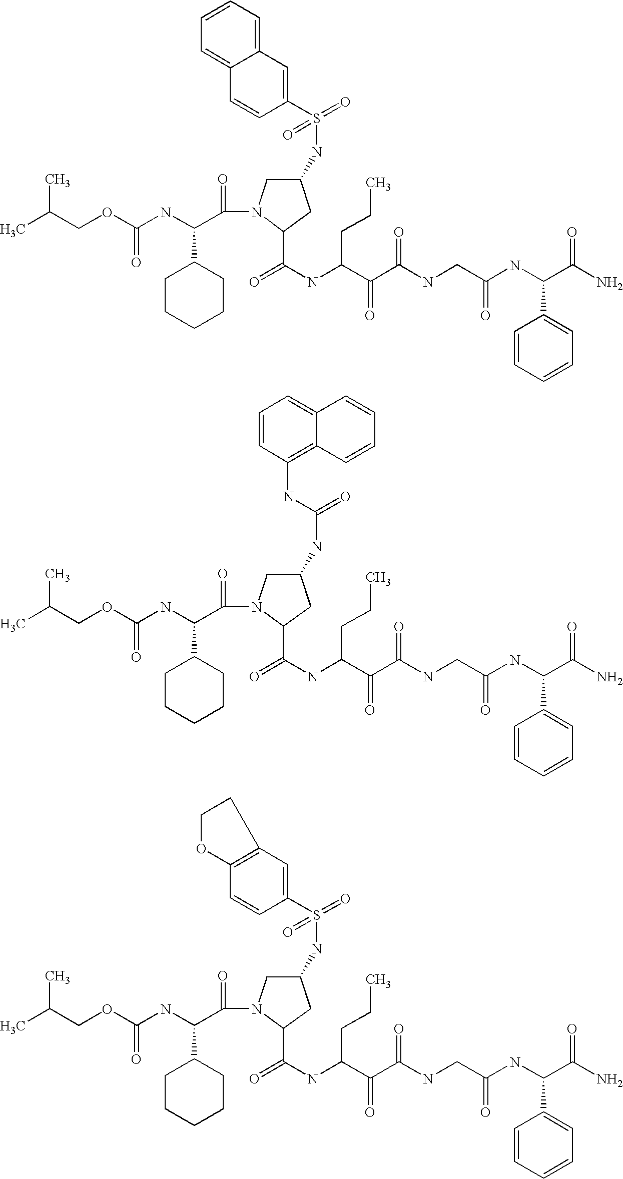 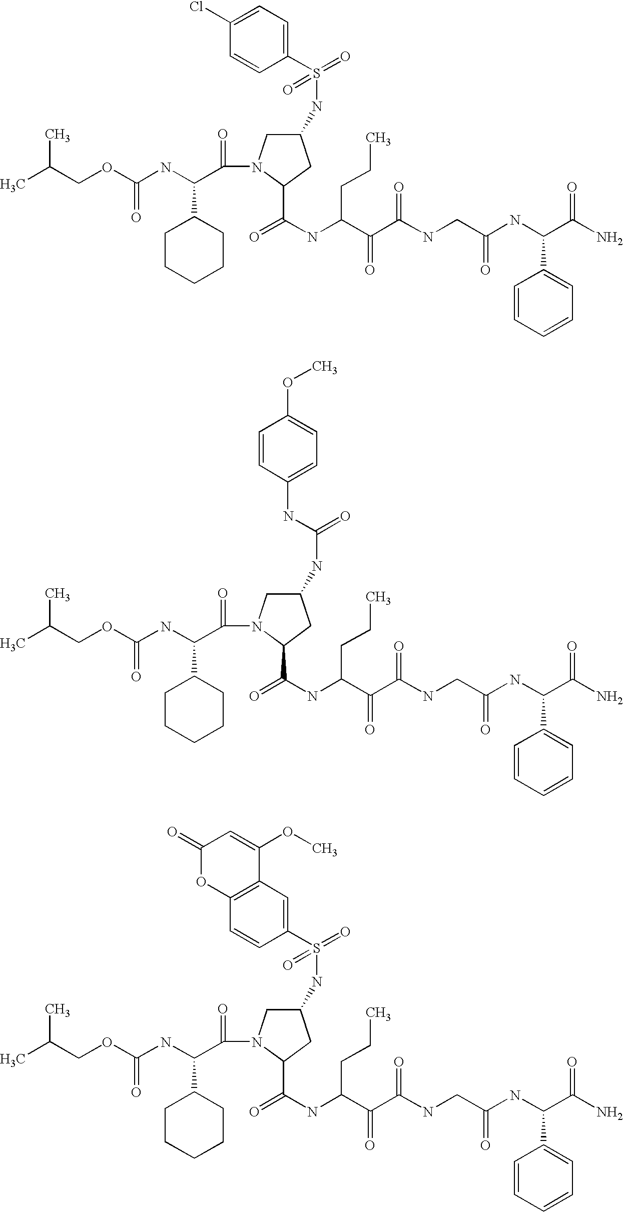 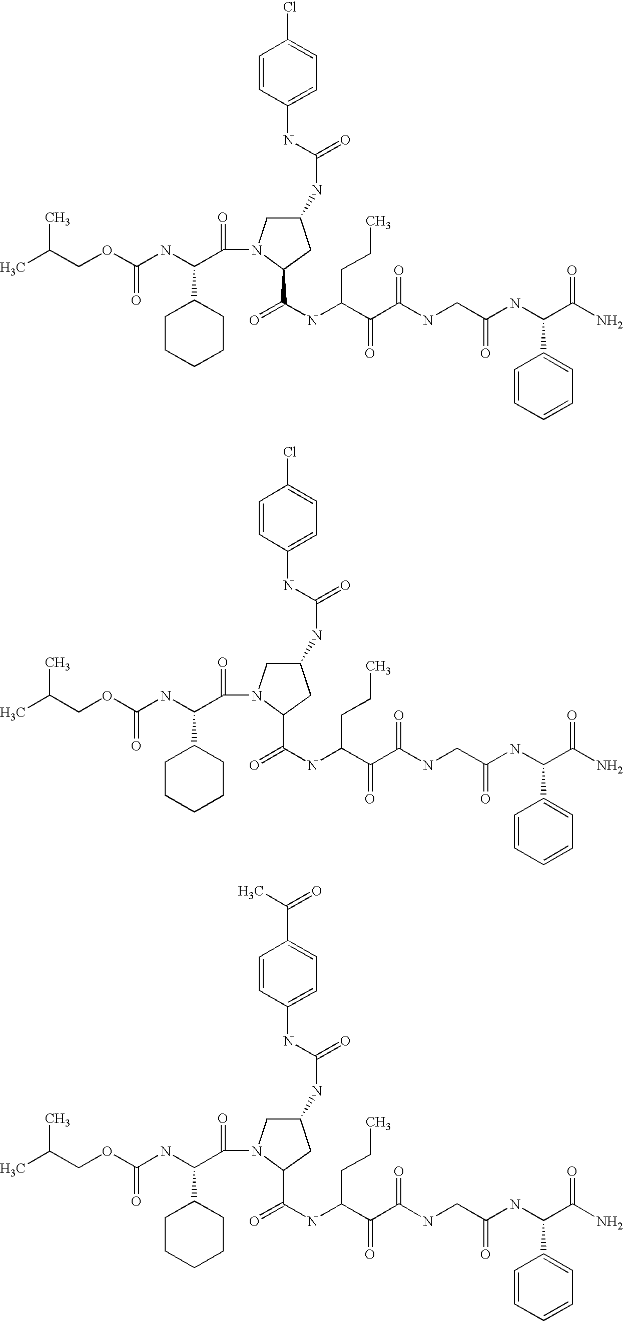 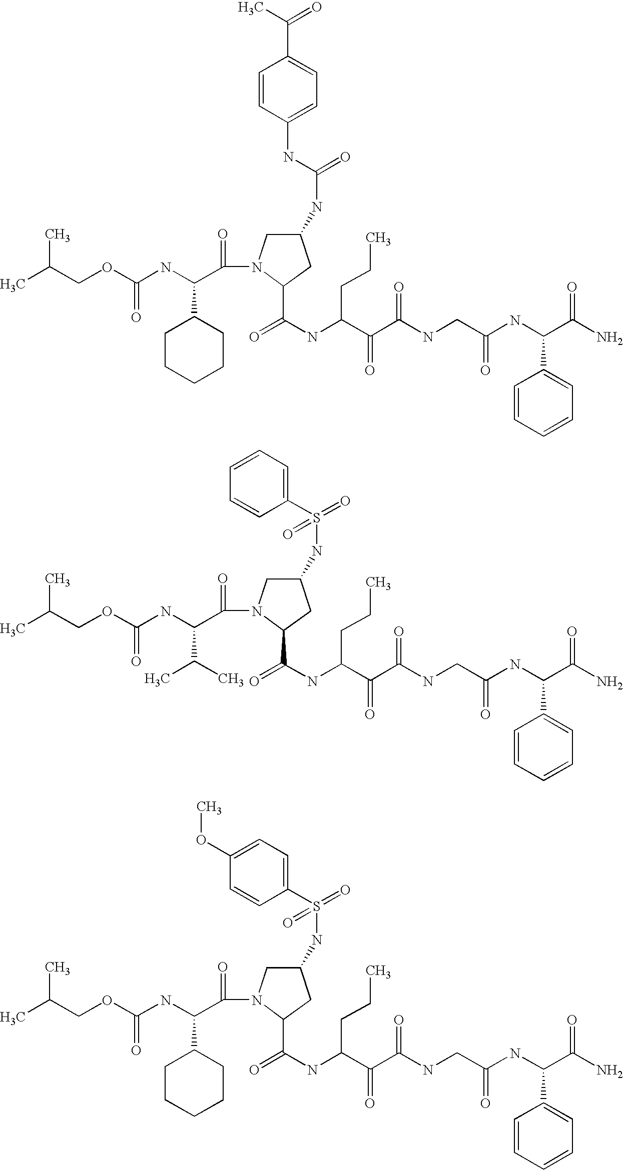 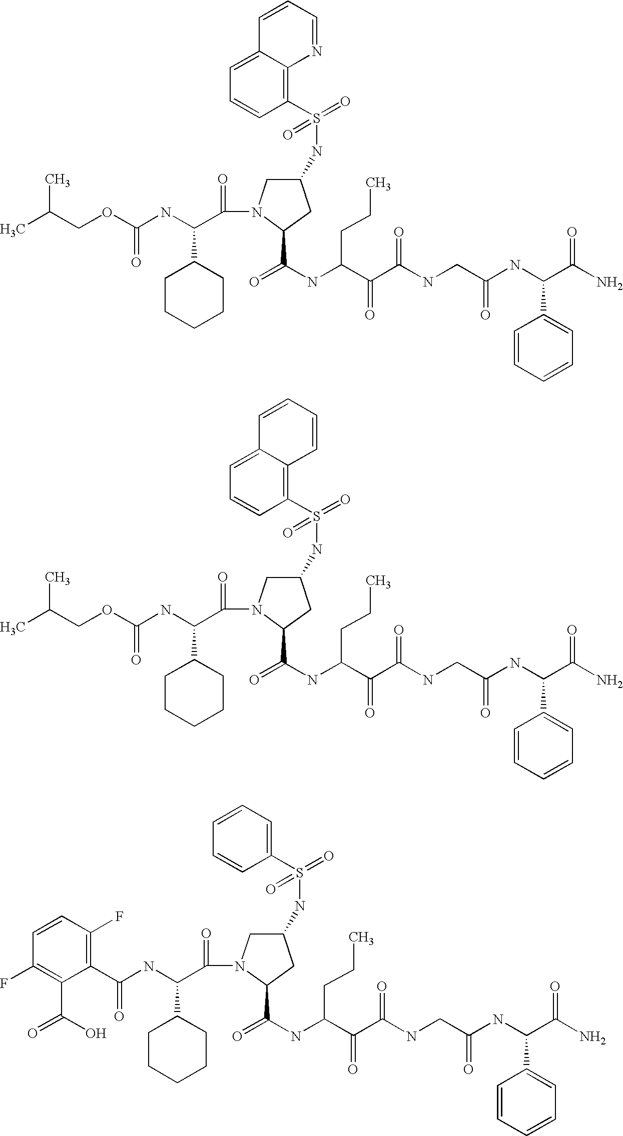 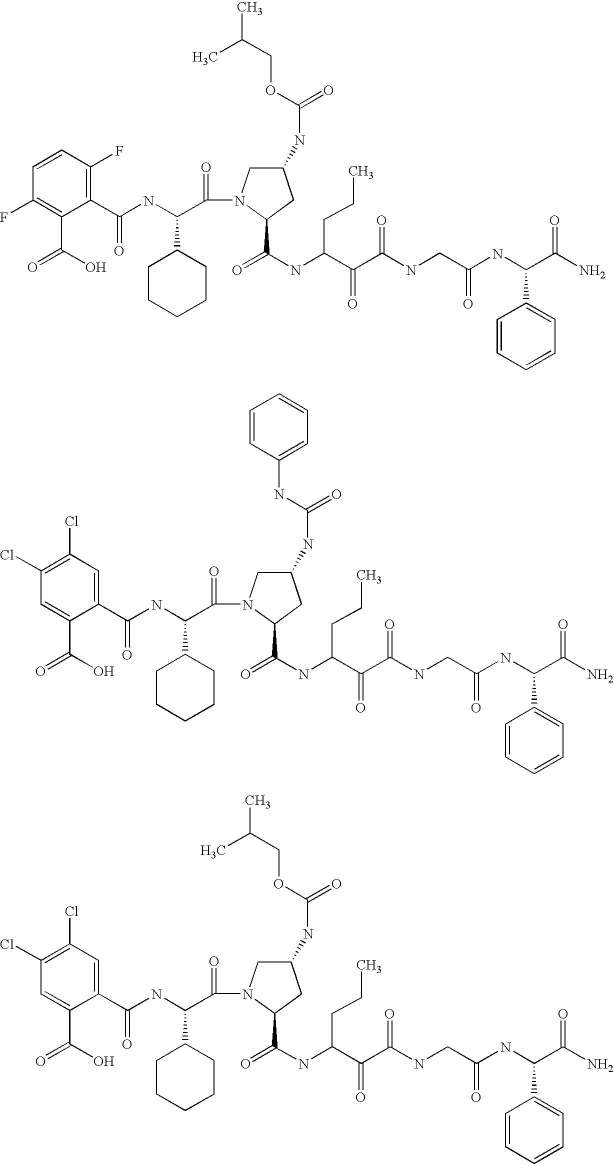 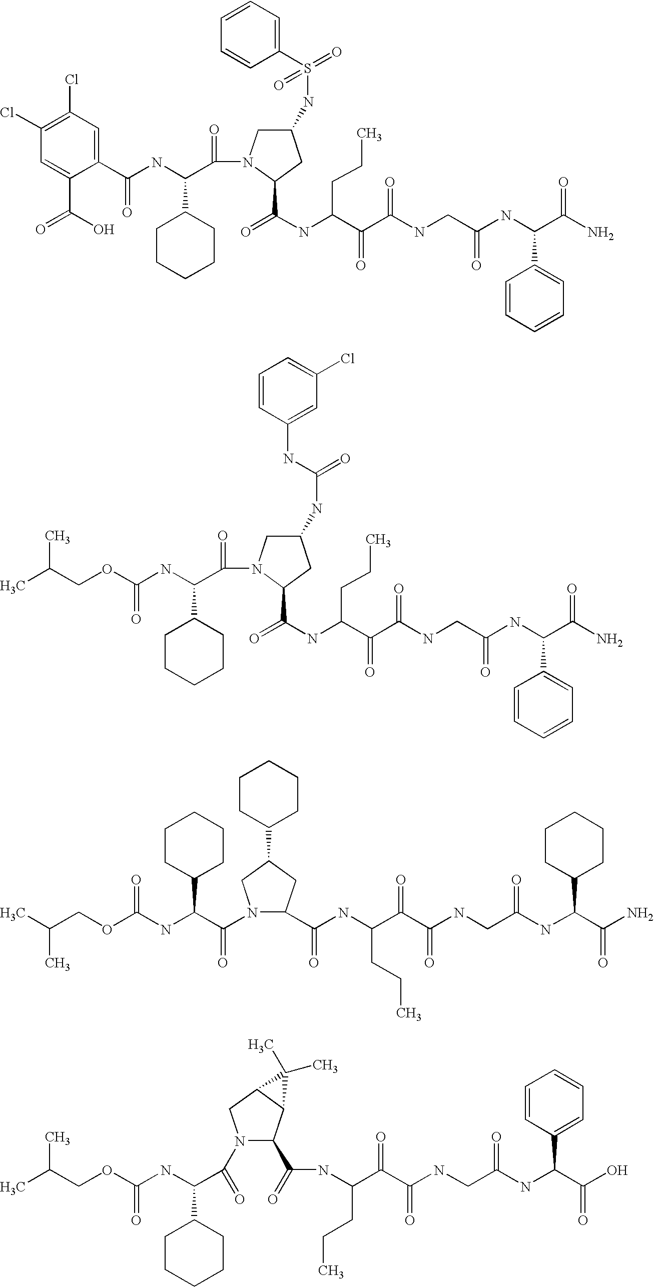 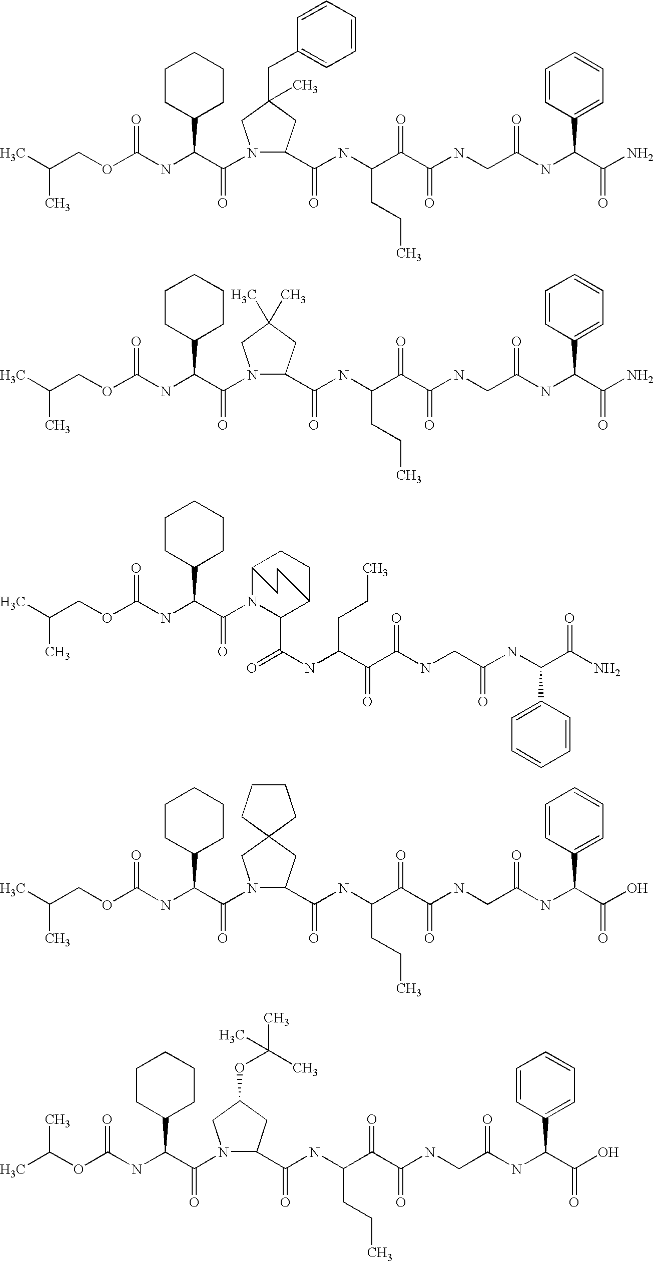 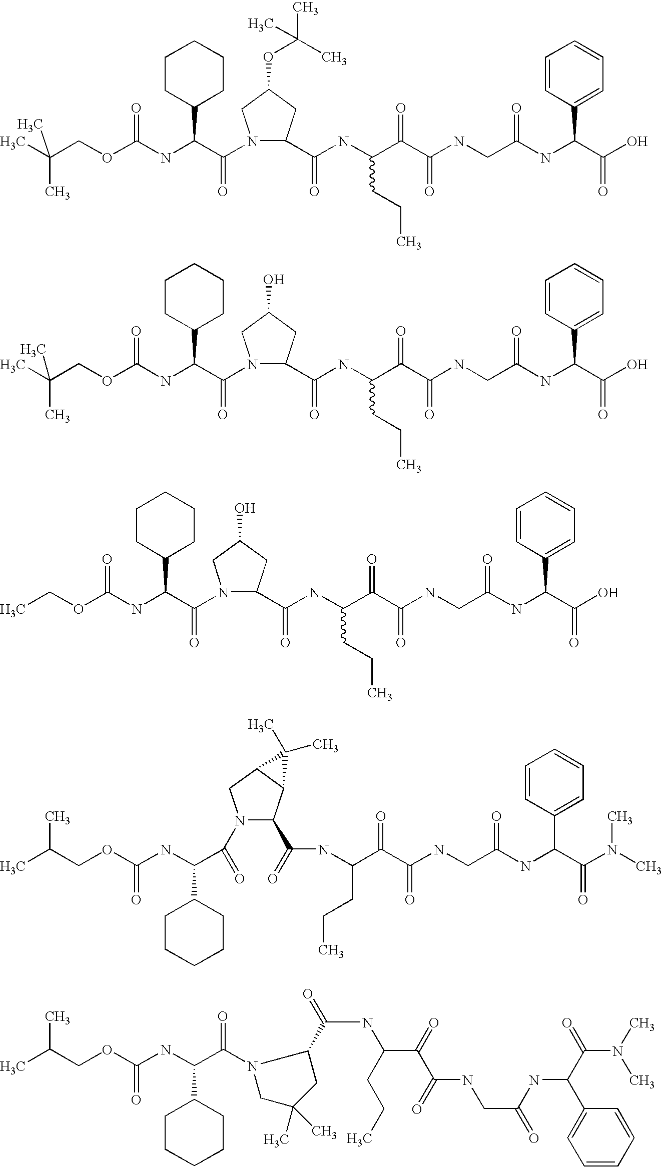 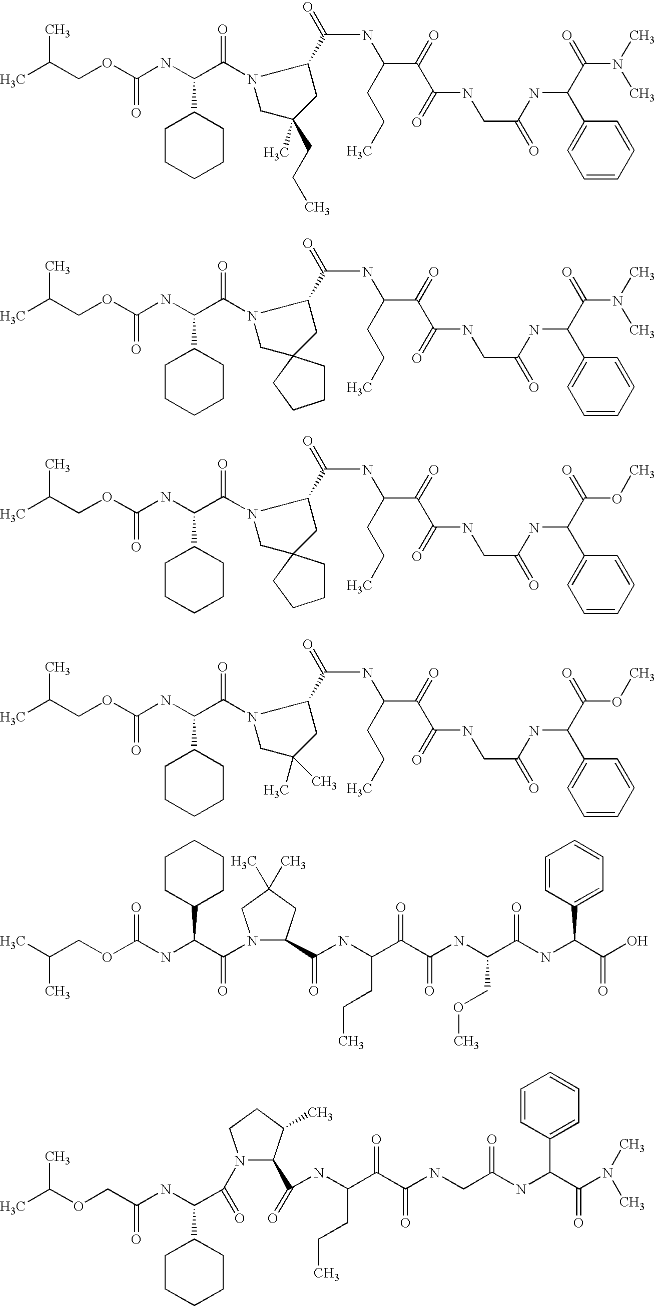 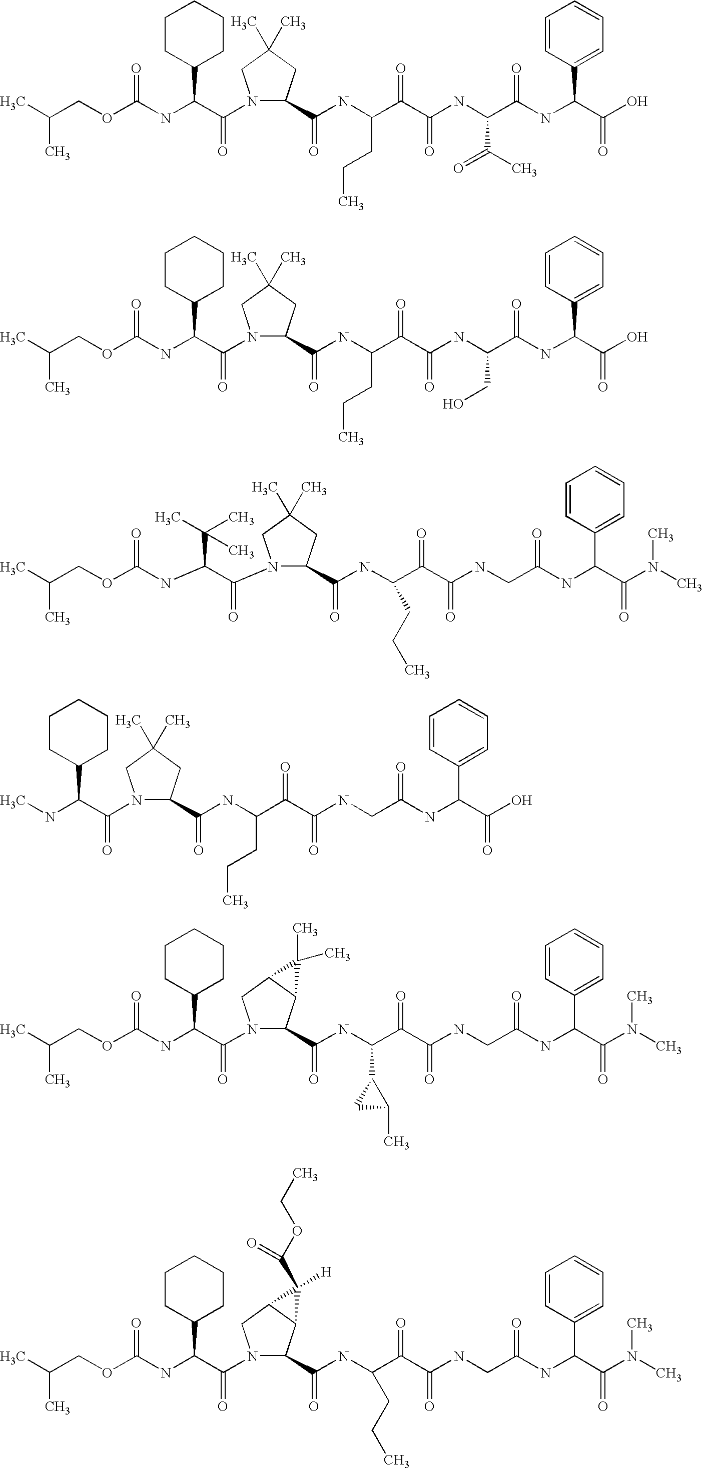 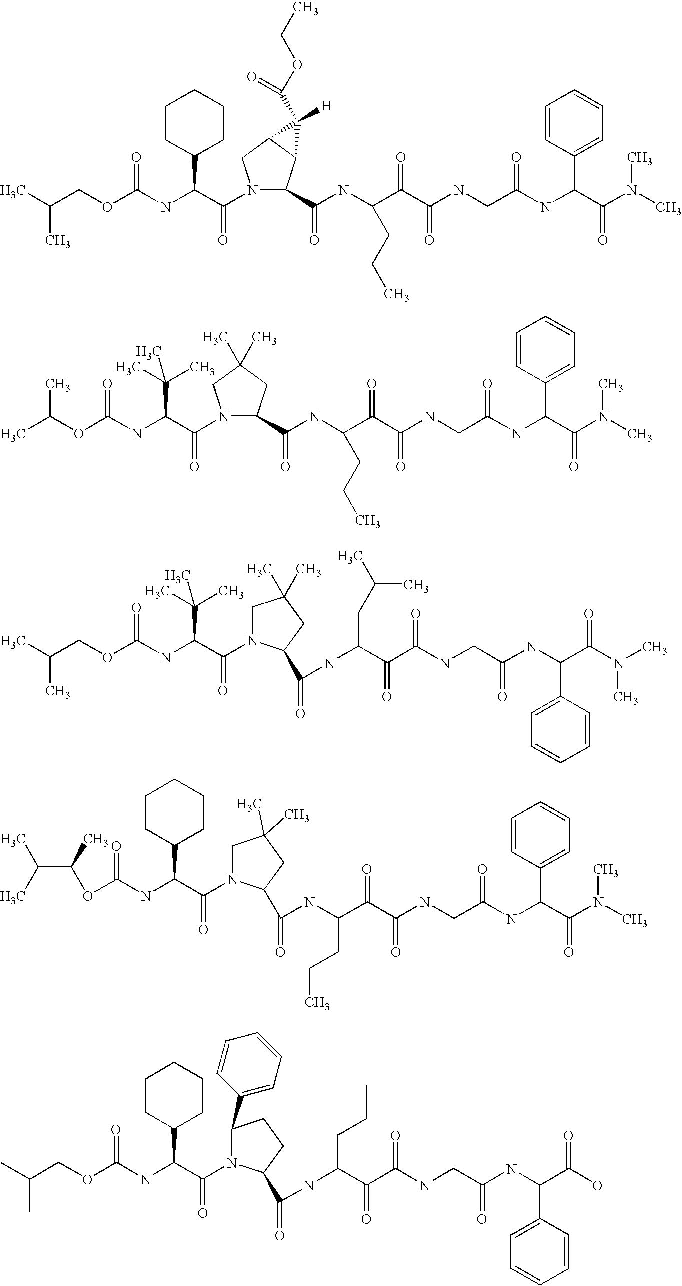 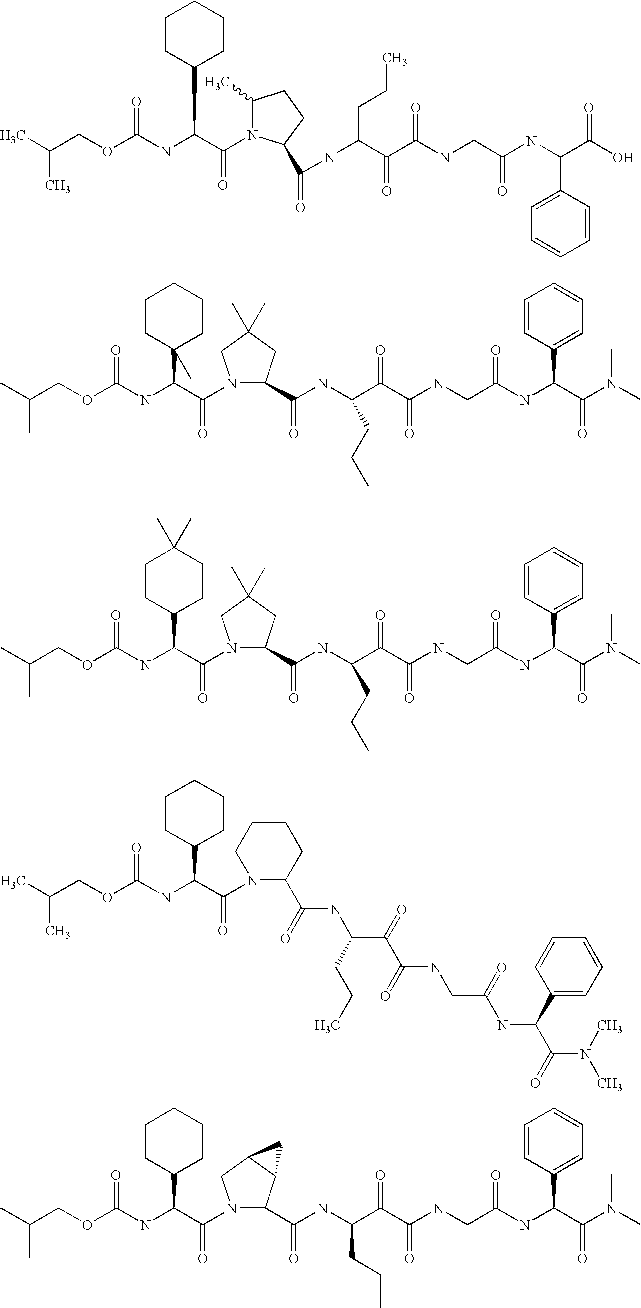 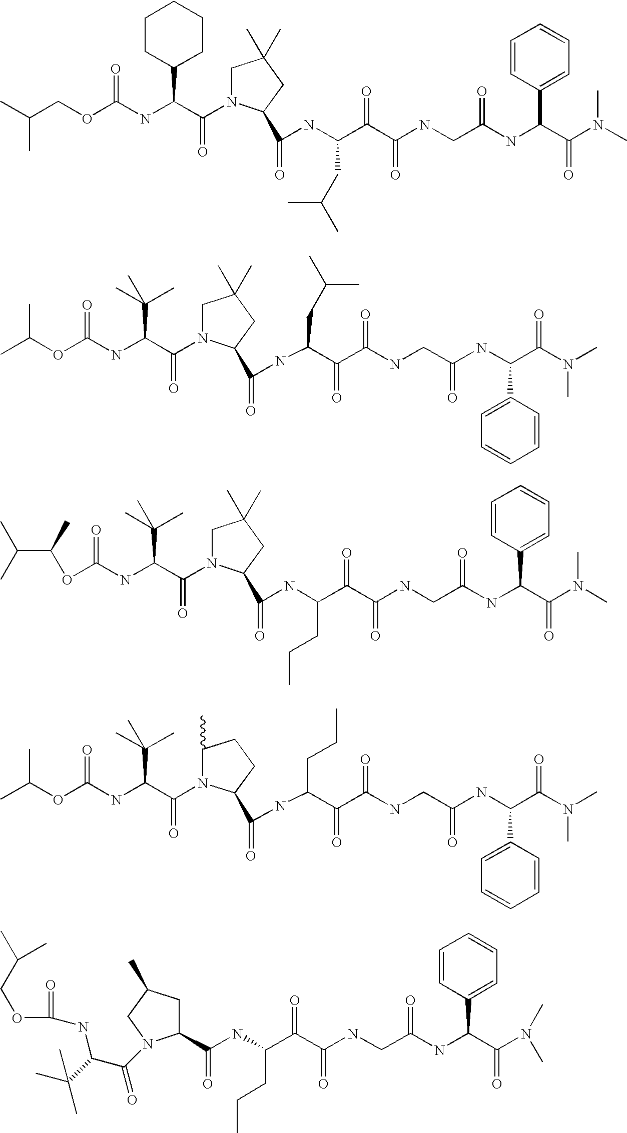 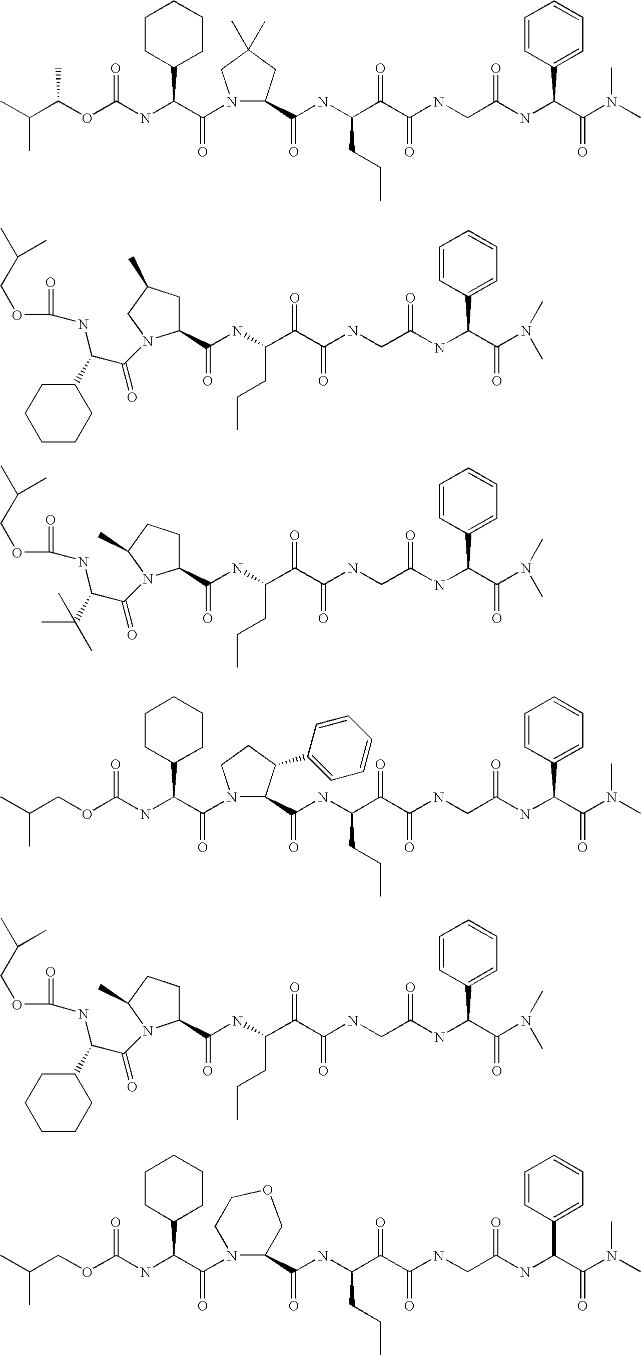 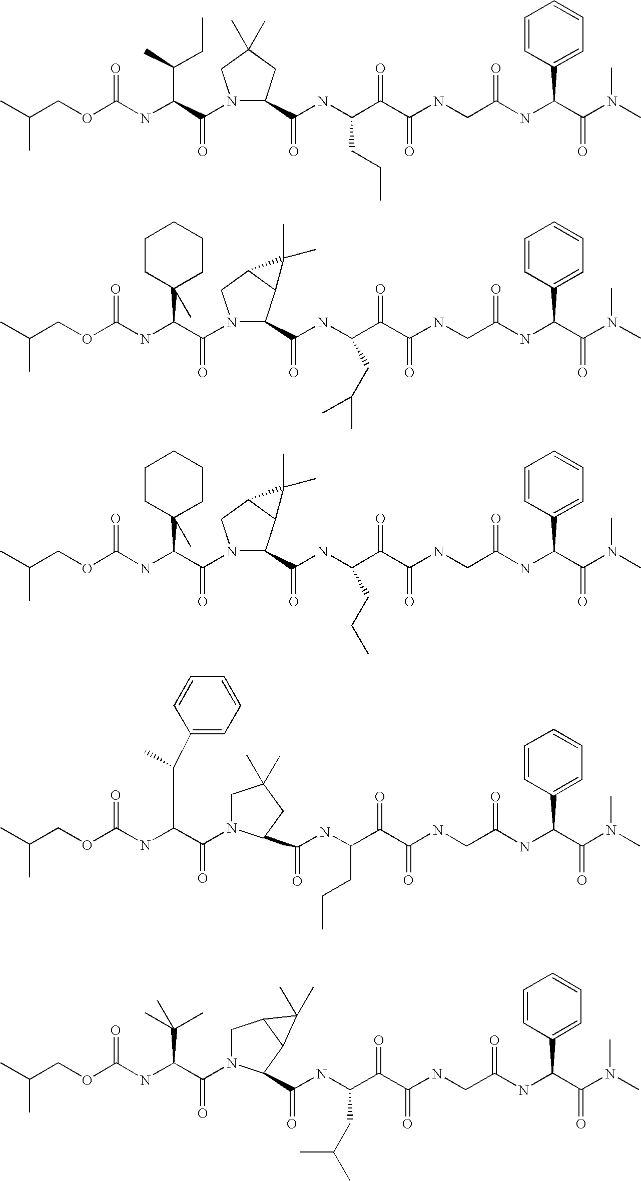 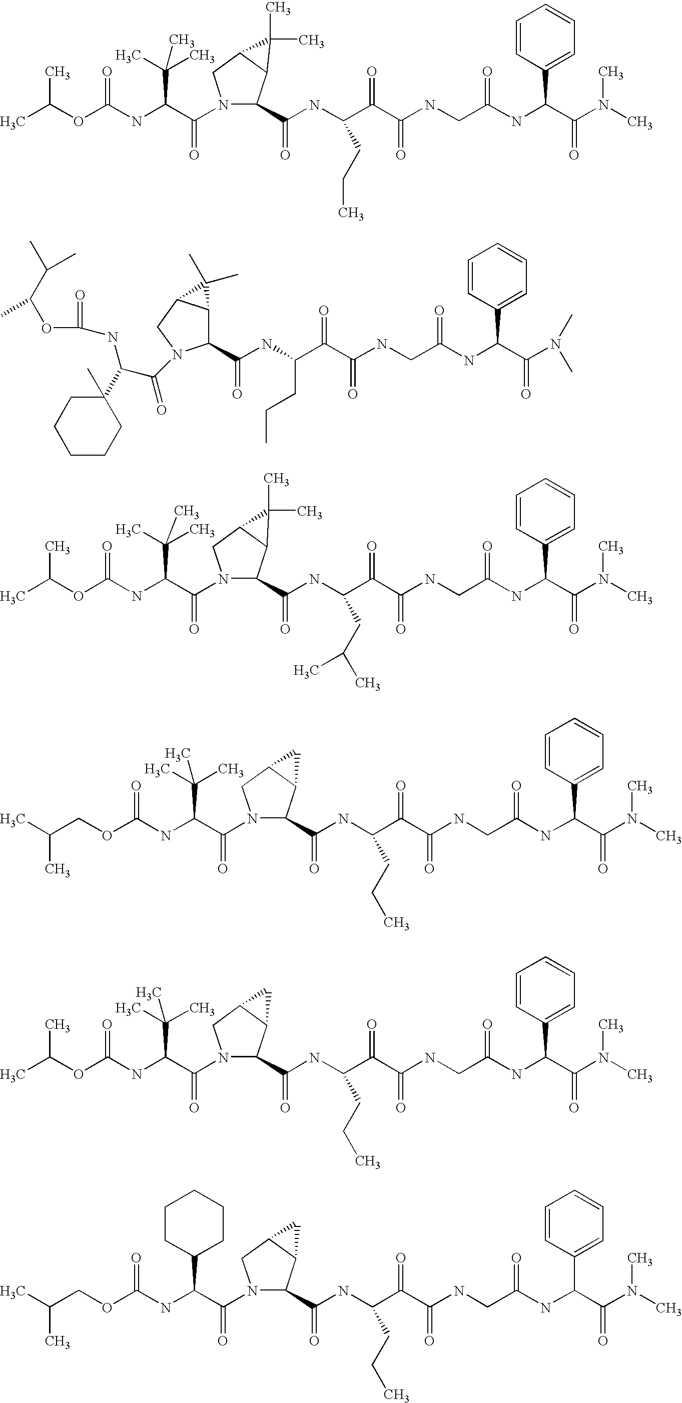 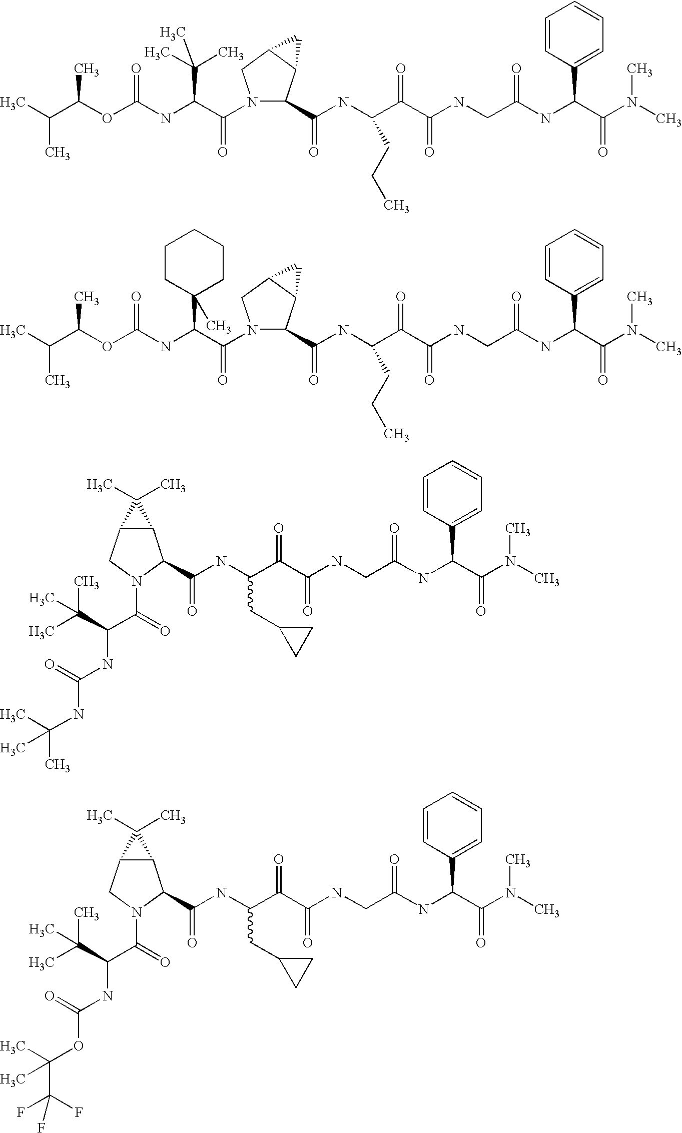 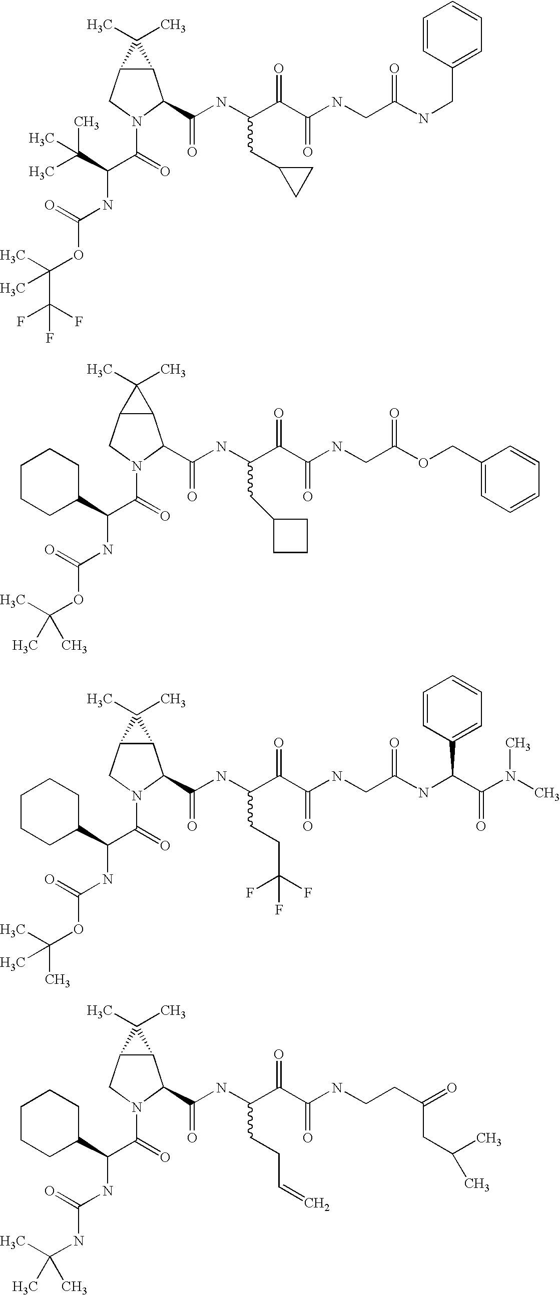 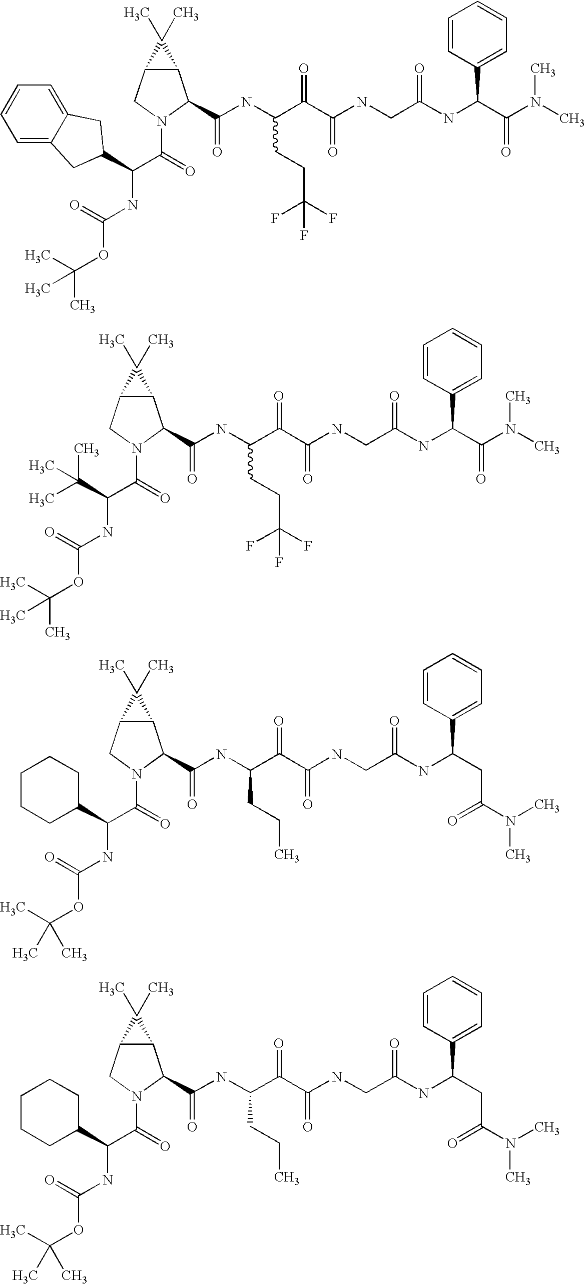 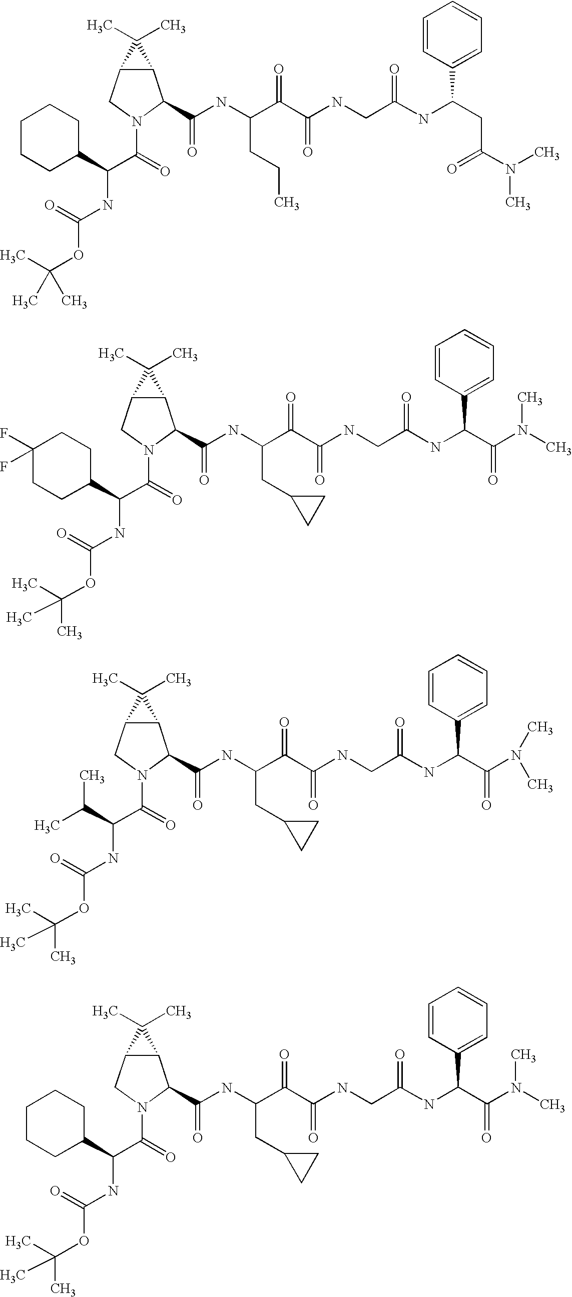 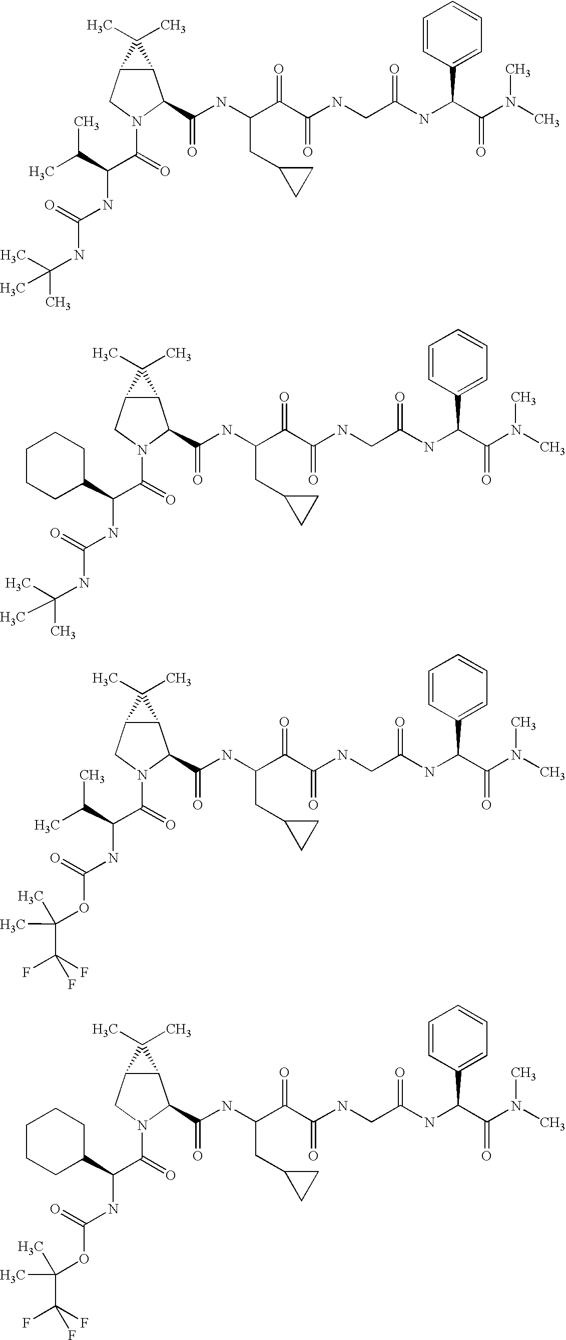 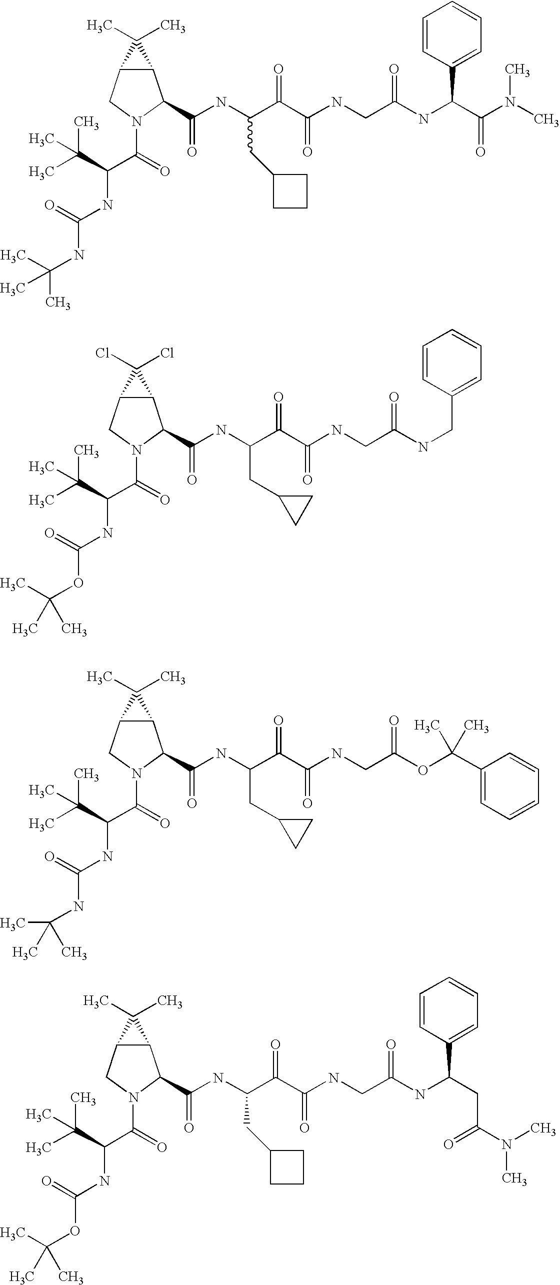 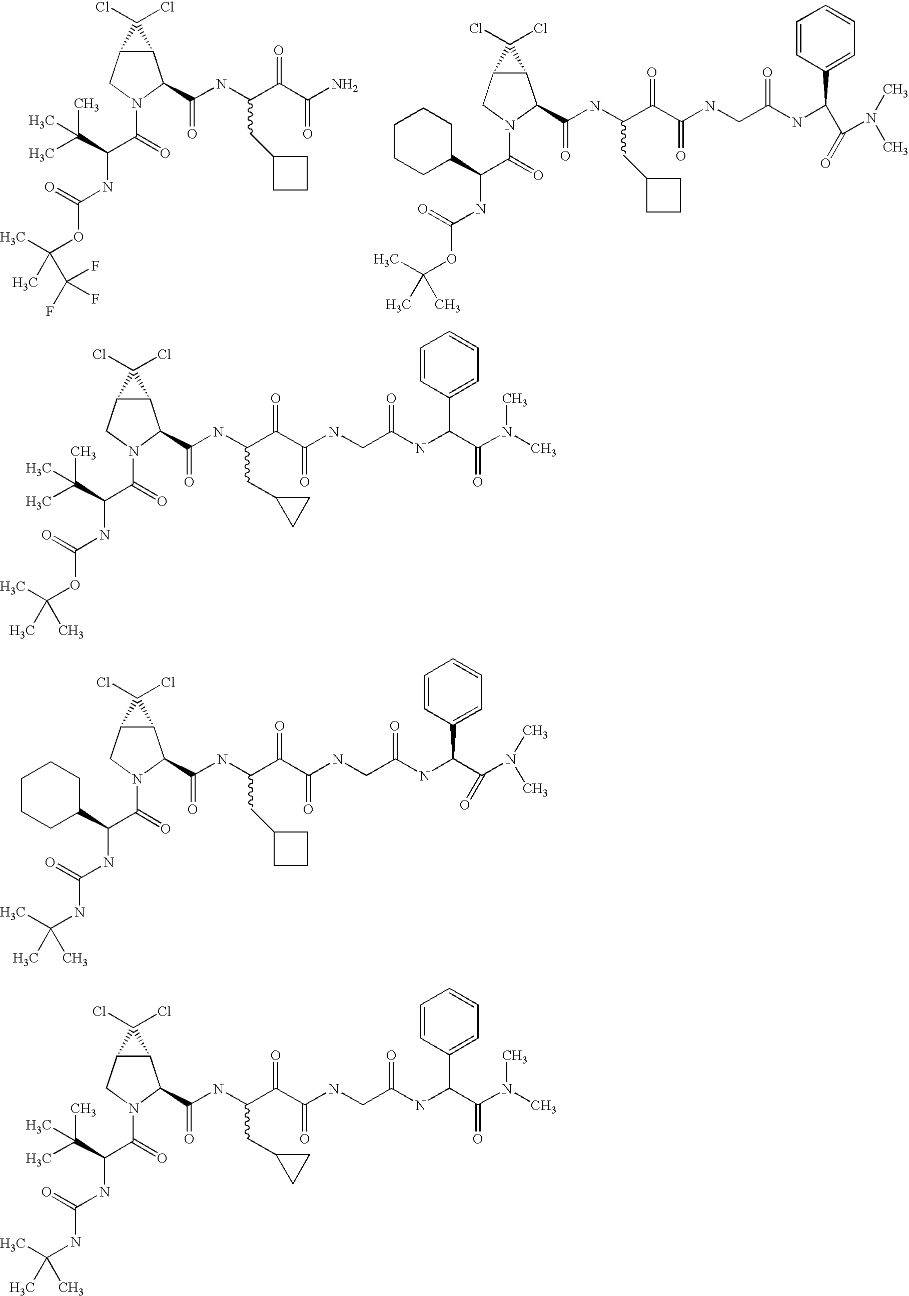 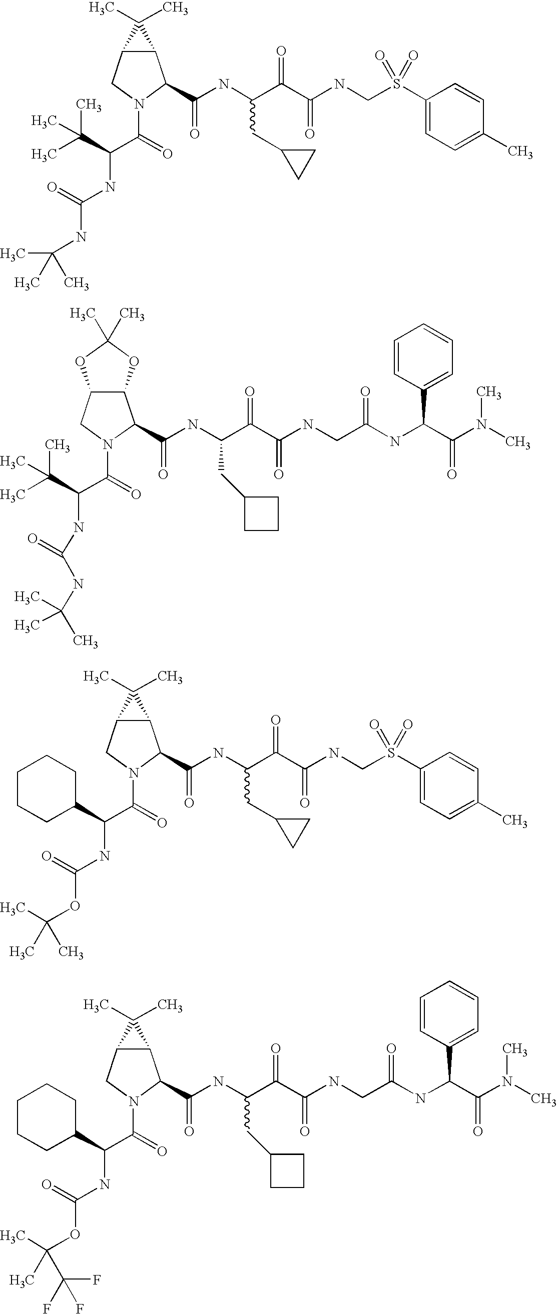 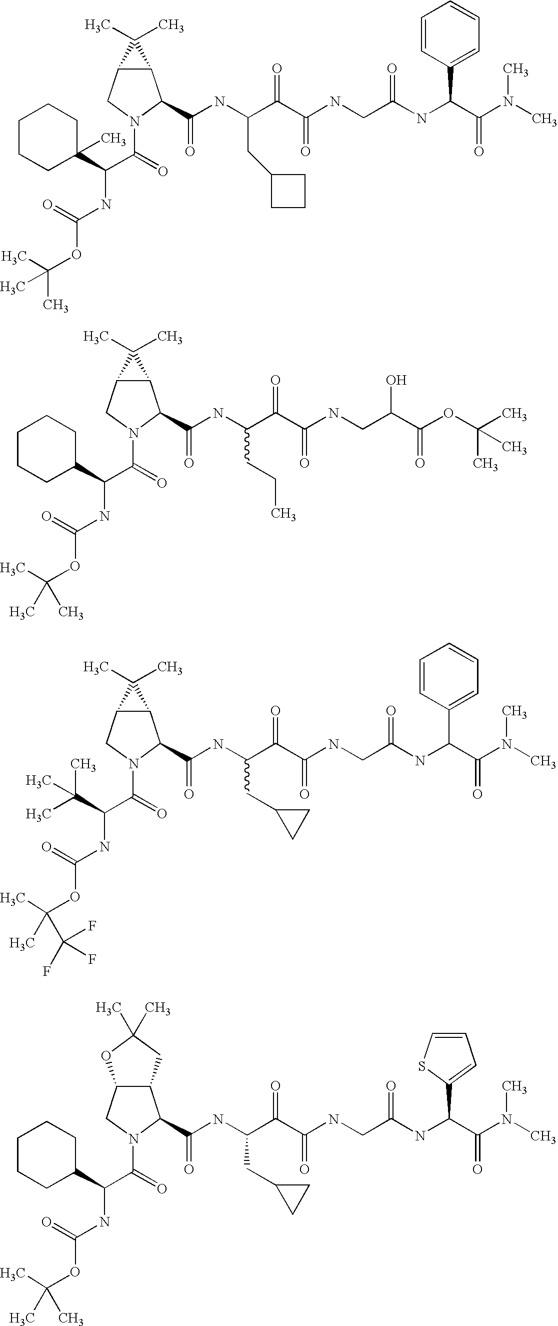 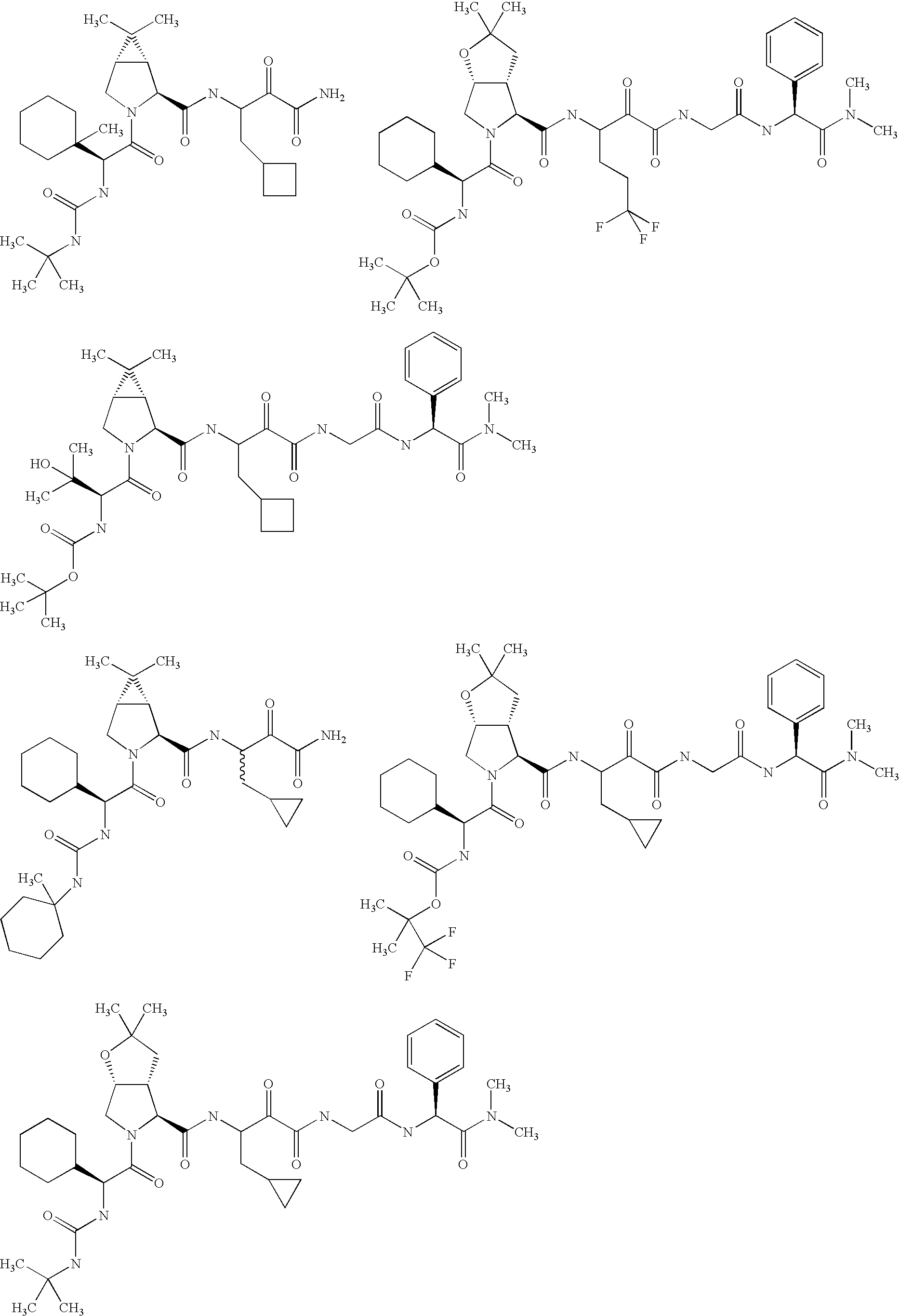 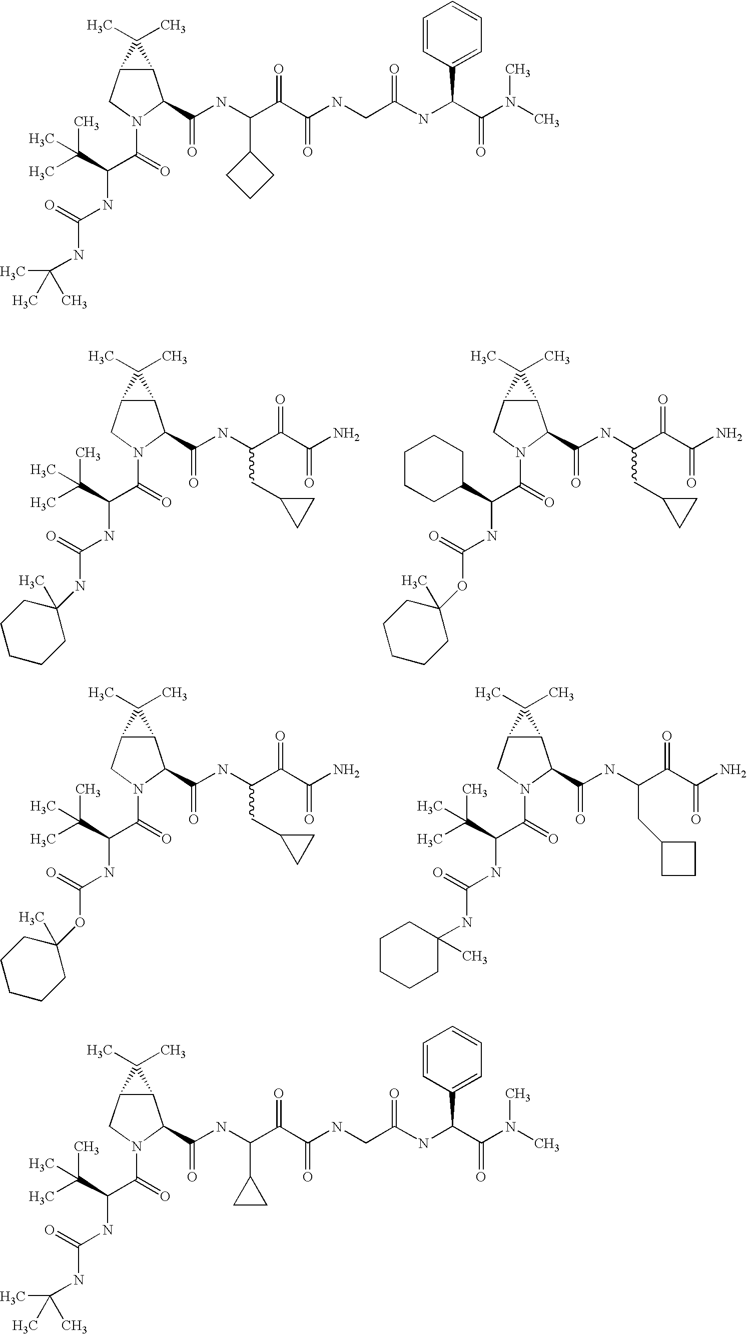 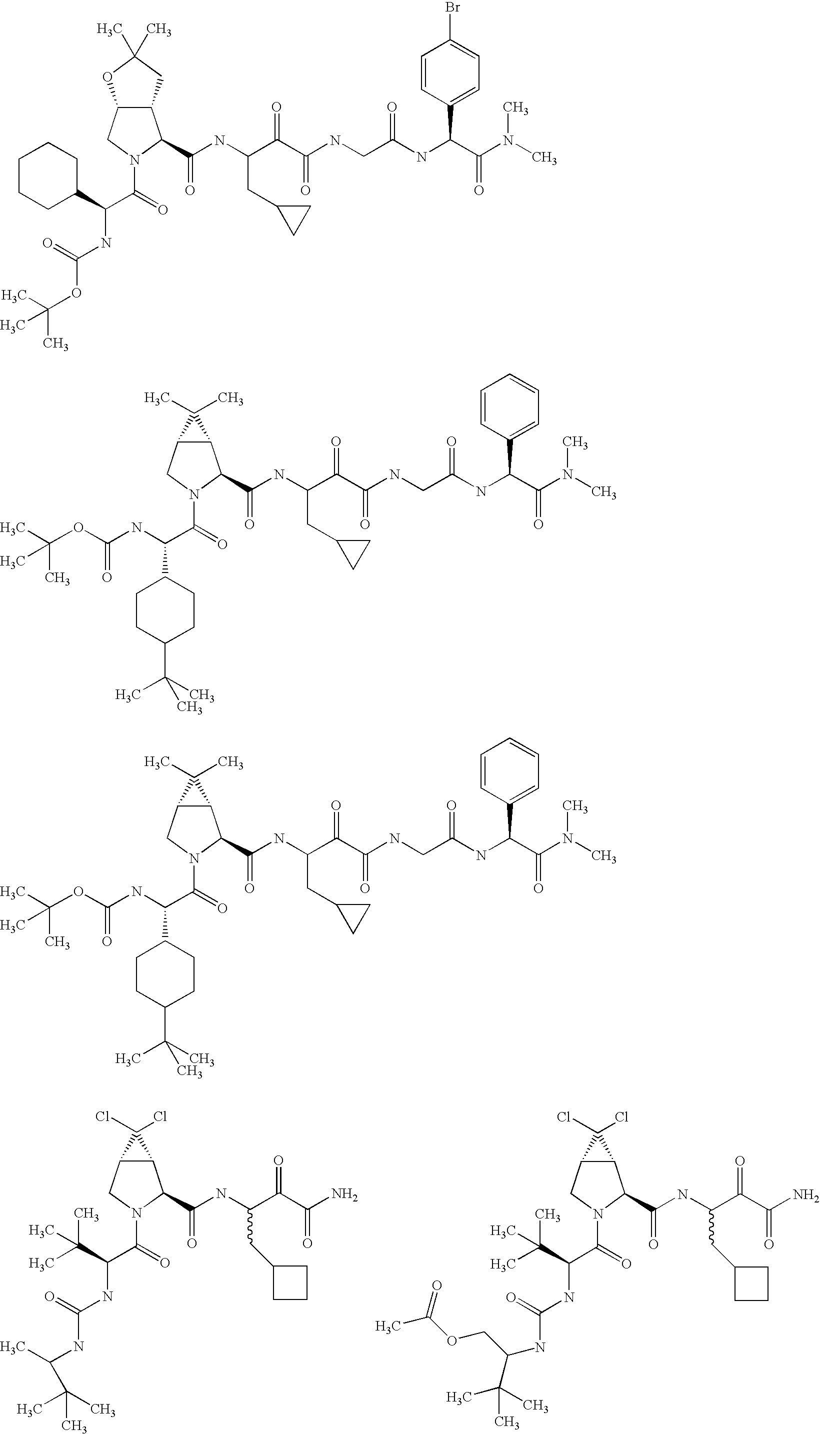 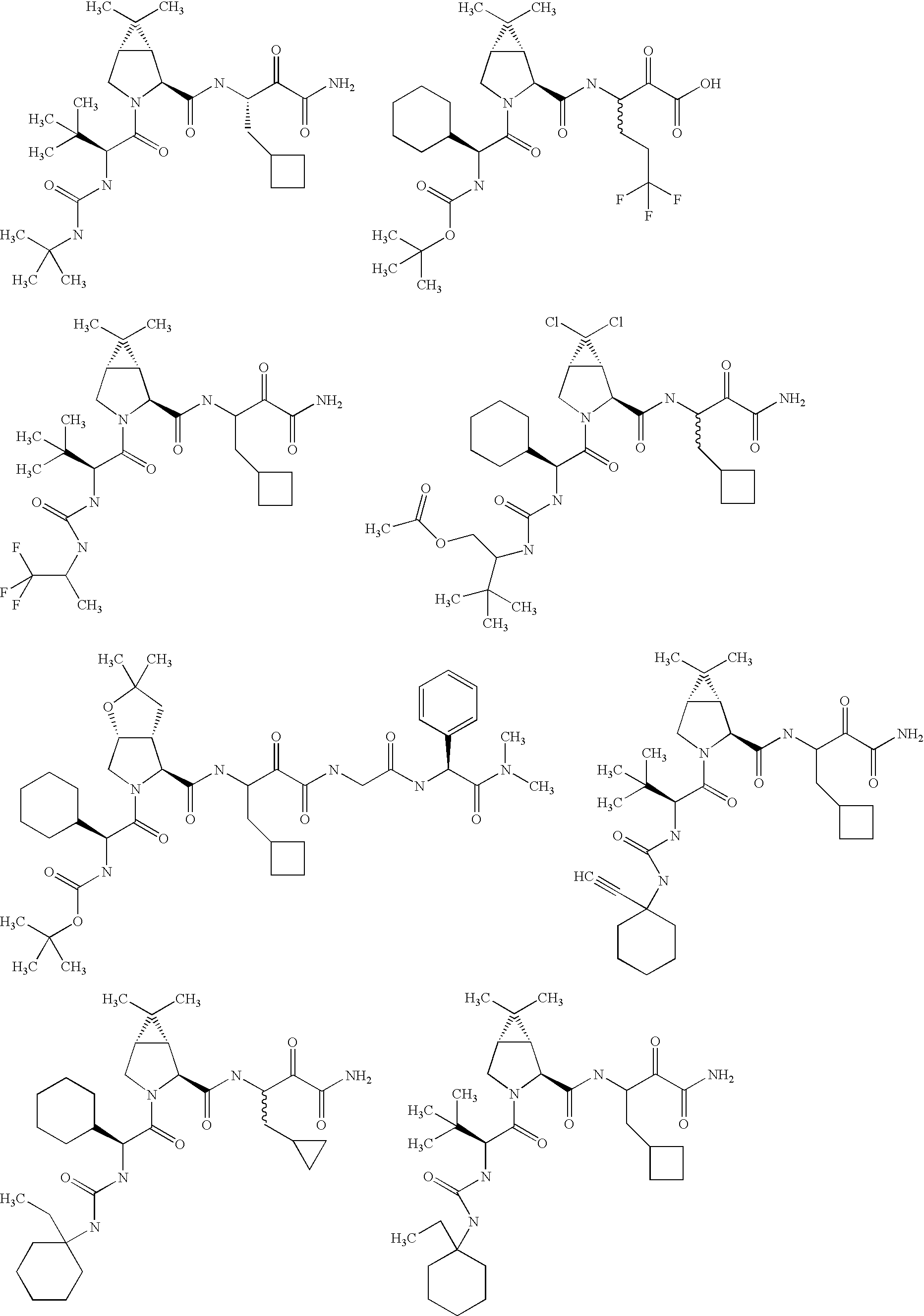 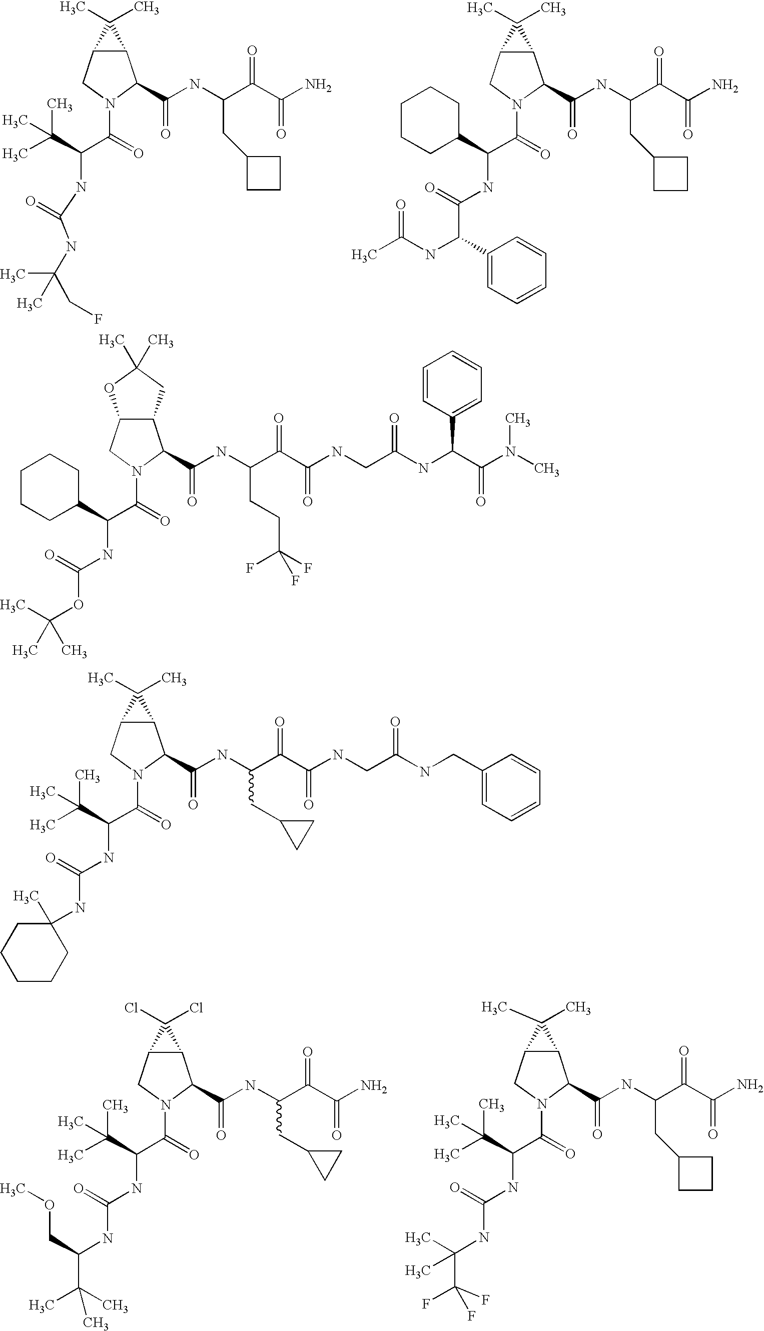 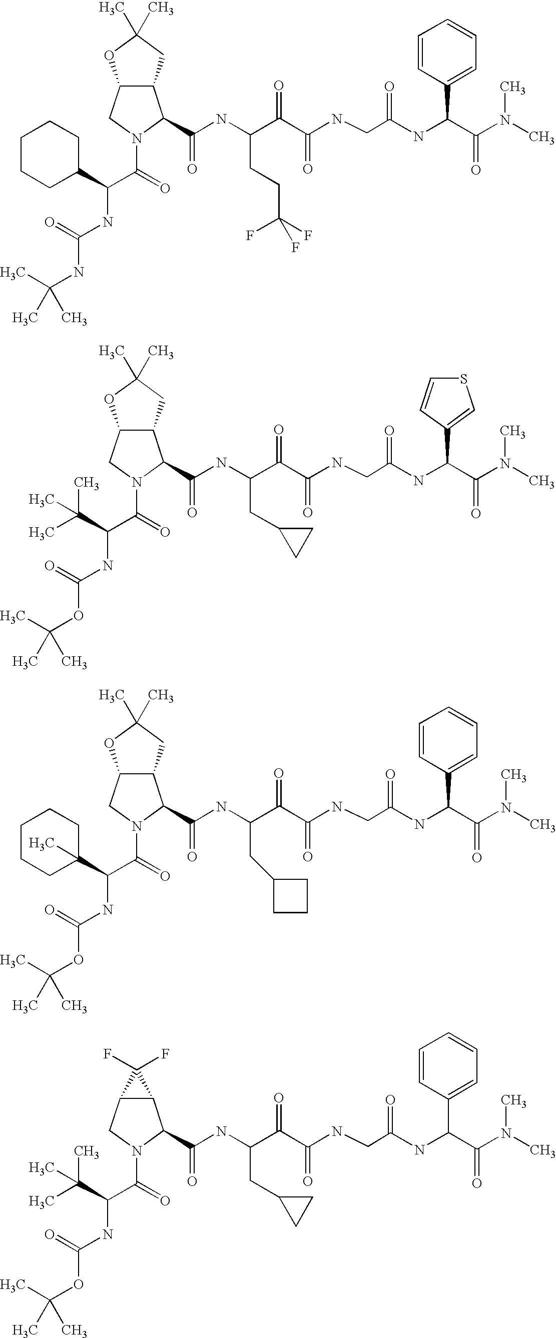 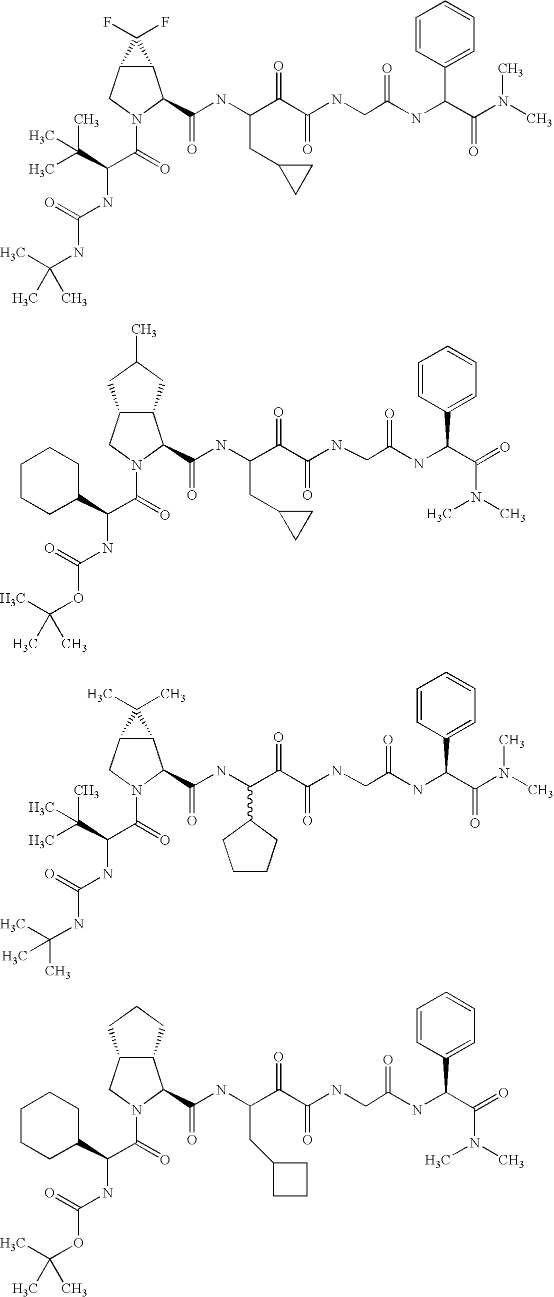 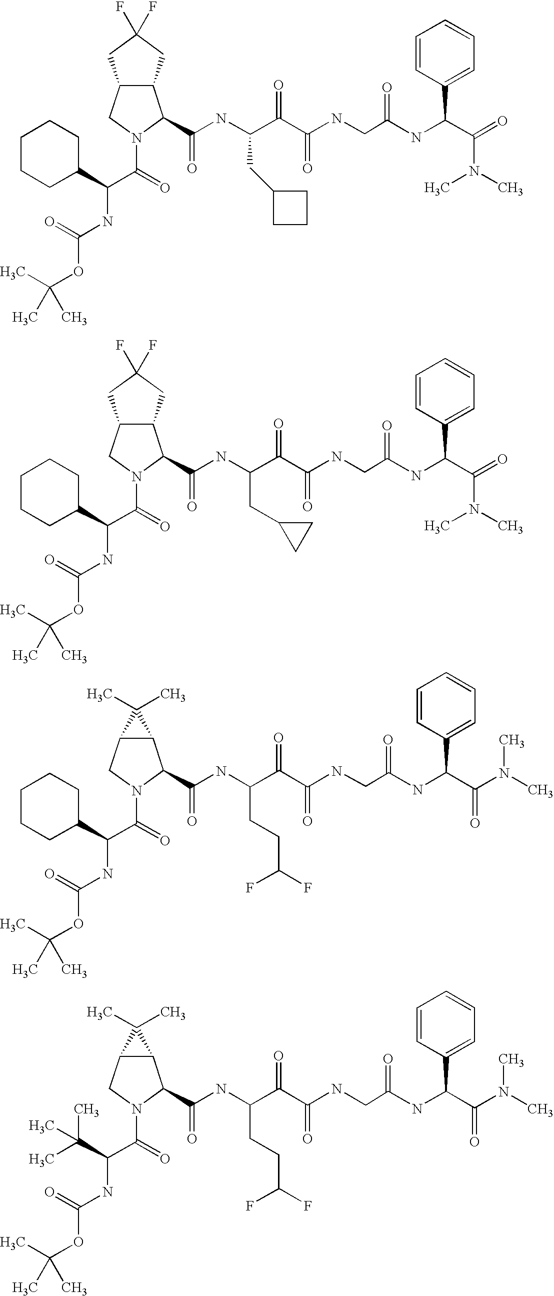 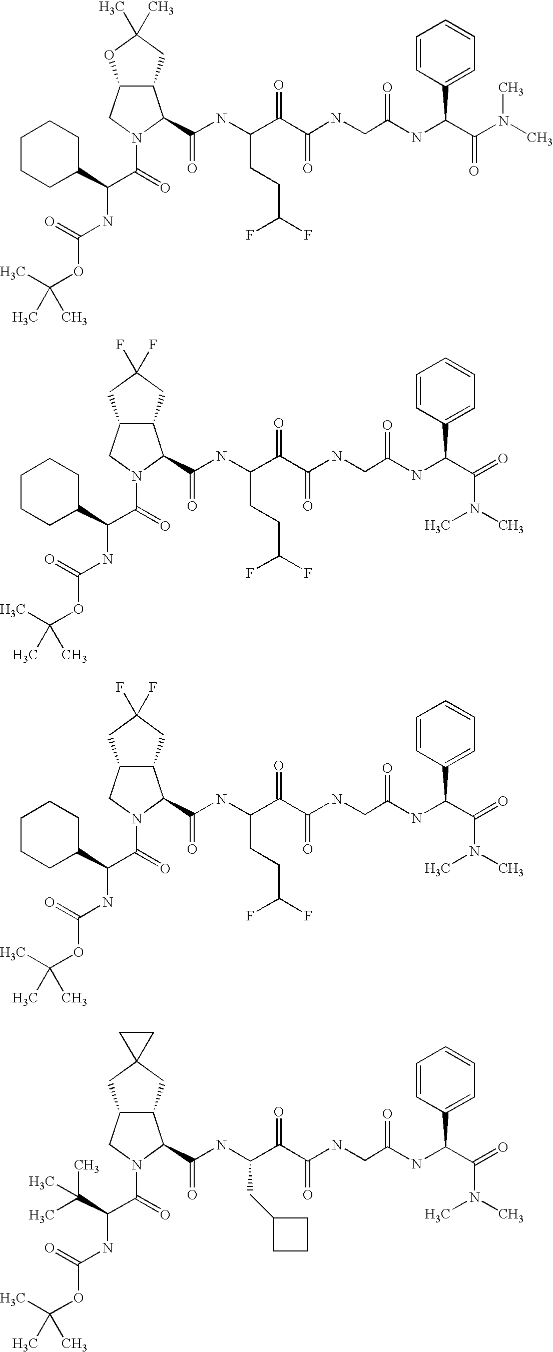 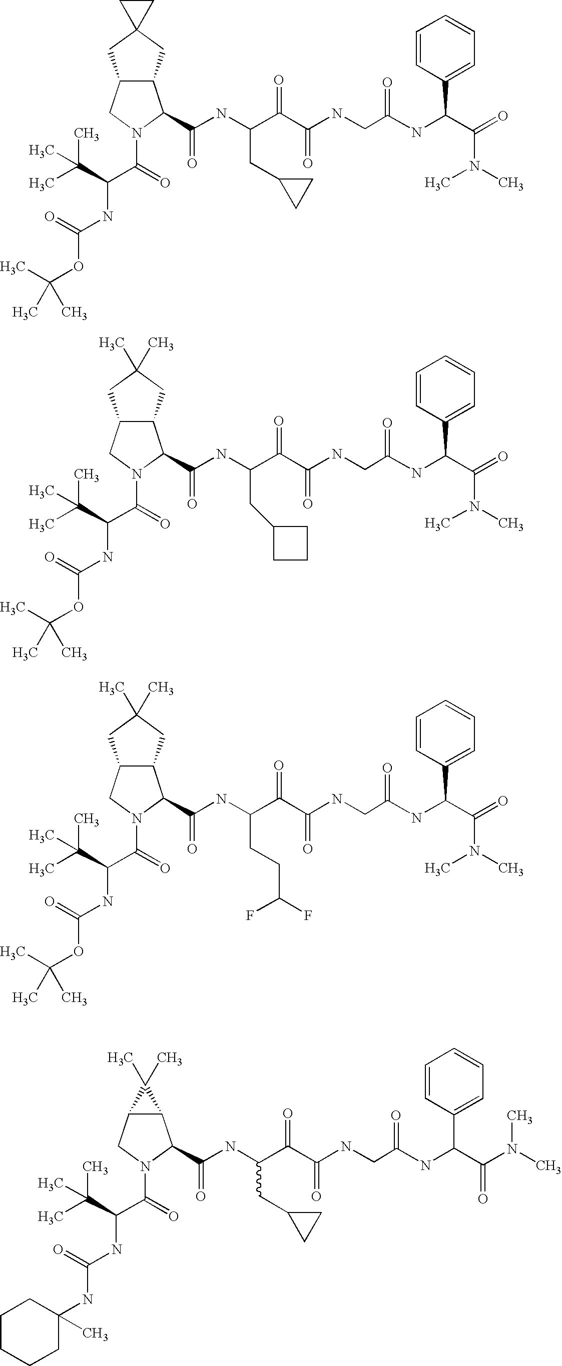 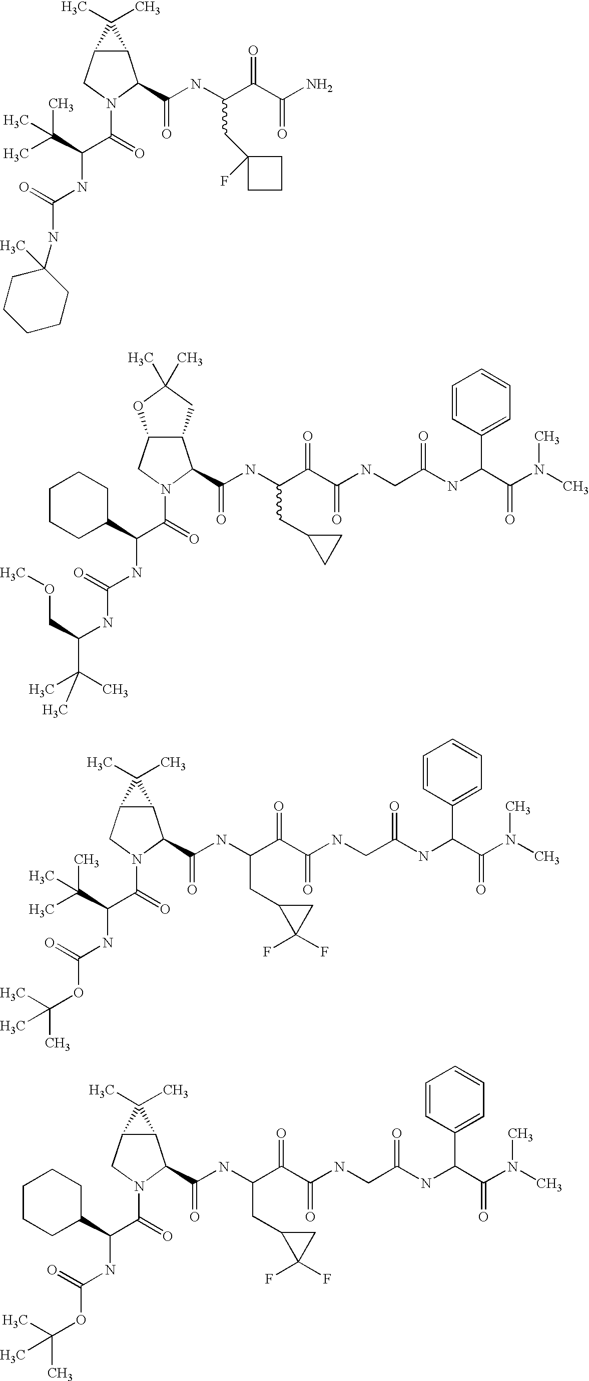 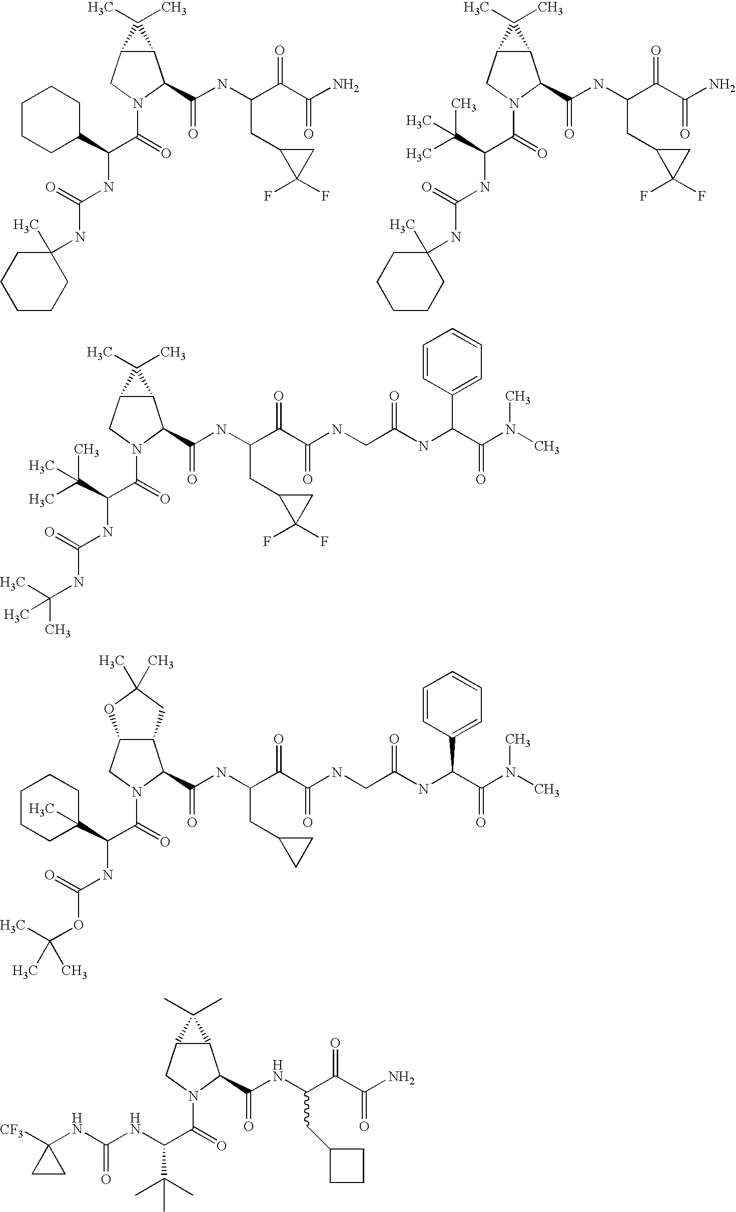
or a pharmaceutically acceptable salt, solvate or ester thereof.

In one embodiment, the HCV protease inhibitor is selected from the group consisting of 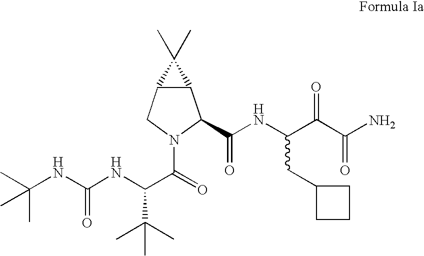
and pharmaceutically acceptable salts or solvates thereof.

The compound of formula Ia has recently been separated into its isomer/diastereomers of Formulas Ib and Ic. In one embodiment, the HCV protease inhibitor is selected from the group consisting of the compound of Formula Ic and pharmaceutically acceptable salts or solvates thereof as a potent inhibitor of HCV NS3 serine protease.

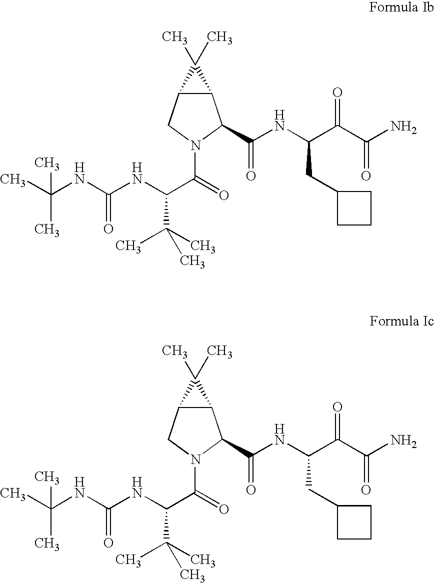

The chemical name of the compound of Formula Ic is (1R,2S,5S)-N-[(1S)-3-amino-1-(cyclobutylmethyl)-2,3-dioxopropyl]-3-[(2S)-2-[[[(1,1-dimethylethyl)amino]carbonyl]amino]-3,3-dimethyl-1-oxobutyl]-6,6-dimethyl-3-azabicyclo[3.1.0]hexane-2-carboxamide.

Processes for making compounds of Formula I are disclosed in U.S. Patent Publication Nos. 2005/0059648, 2005/0020689 and 2005/0059800, incorporated by reference herein.

Non-limiting examples of suitable compounds of formula II and methods of making the same are disclosed in WO02/08256 and in U.S. Pat. No. 6,800,434, at col. 5 through col. 247, incorporated herein by reference.

Non-limiting examples of suitable compounds of formula III and methods of making the same are disclosed in International Patent Publication WO02/08187 and in U.S. Patent Publication 2002/0160962 at page 3, paragraph 22 through page 132, incorporated herein by reference.

Non-limiting examples of suitable compounds of formula IV and methods of making the same are disclosed in International Patent Publication WO03/062228 and in U.S. Patent Publication 2003/0207861 at page 3, paragraph 25 through page 26, incorporated herein by reference.

Non-limiting examples of suitable compounds of formula V and methods of making the same are disclosed in U.S. patent application Ser. No. 10/948,367 filed Sep. 23, 2004, and the preparation of the compounds are detailed in the experimental section of this application set forth hereinbelow.

Non-limiting examples of suitable compounds of formula VI and methods of making the same are disclosed in U.S. Patent Publication Ser. No. 2005/0085425 at page 3, paragraph 0023 through page 139, incorporated herein by reference.

Compounds of formula VII-IX are disclosed in U.S. patent application Ser. No. 10/993,394 filed Nov. 19, 2004, and the preparation of the compounds are detailed in the experimental section of this application set forth hereinbelow.

Non-limiting examples of certain compounds of formula VII disclosed in U.S. patent application Ser. No. 10/993,394 are: 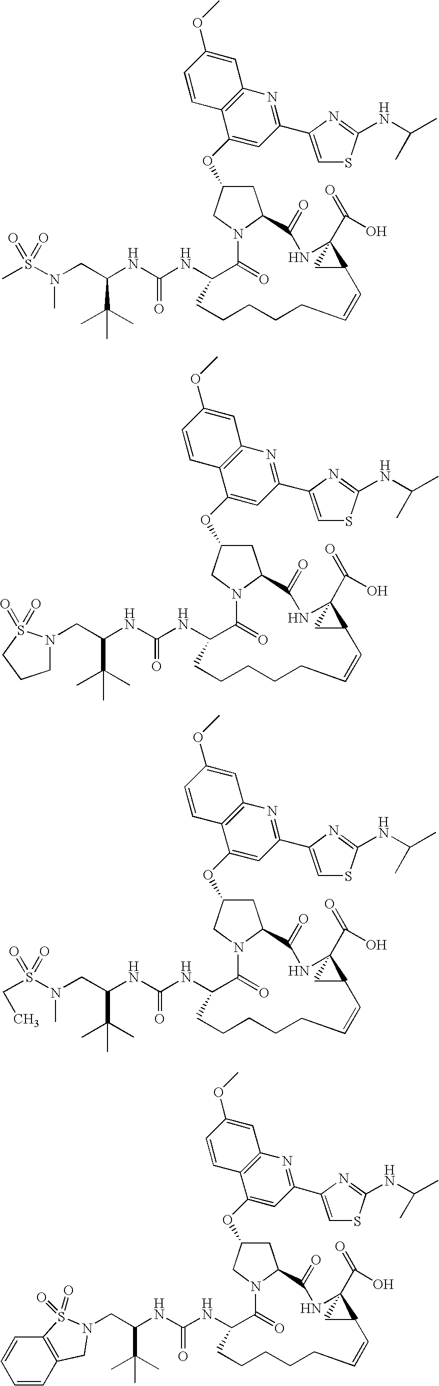 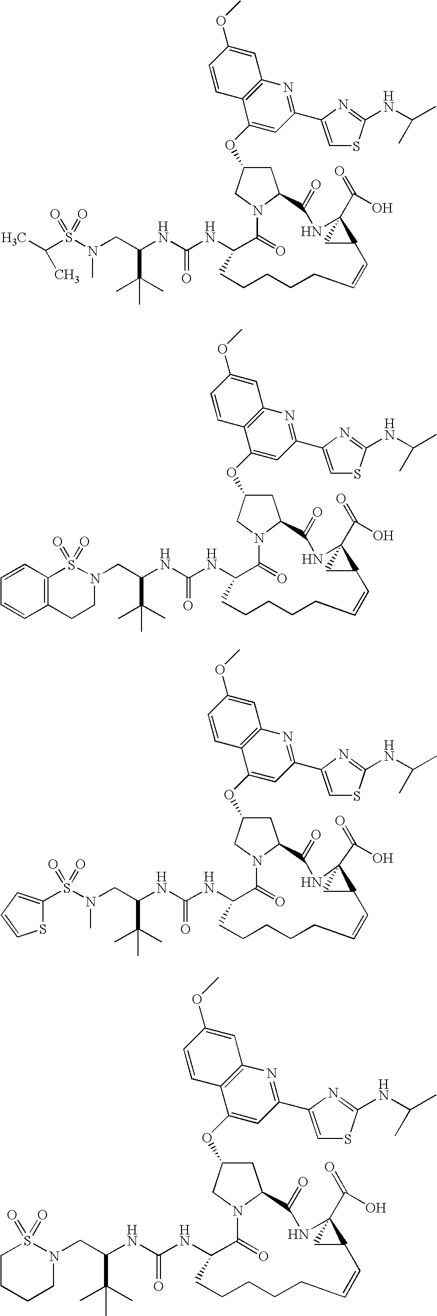 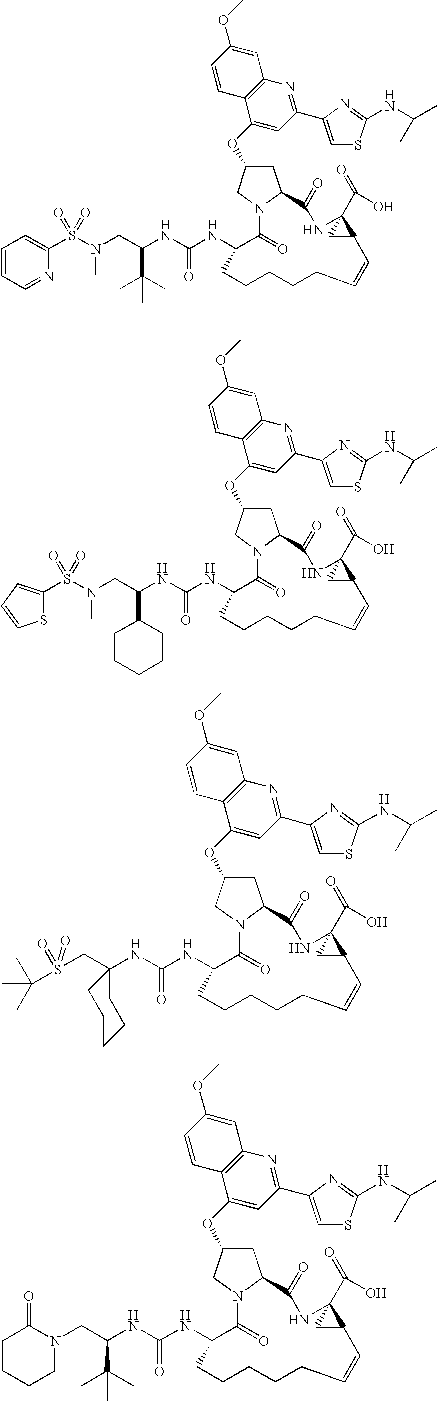 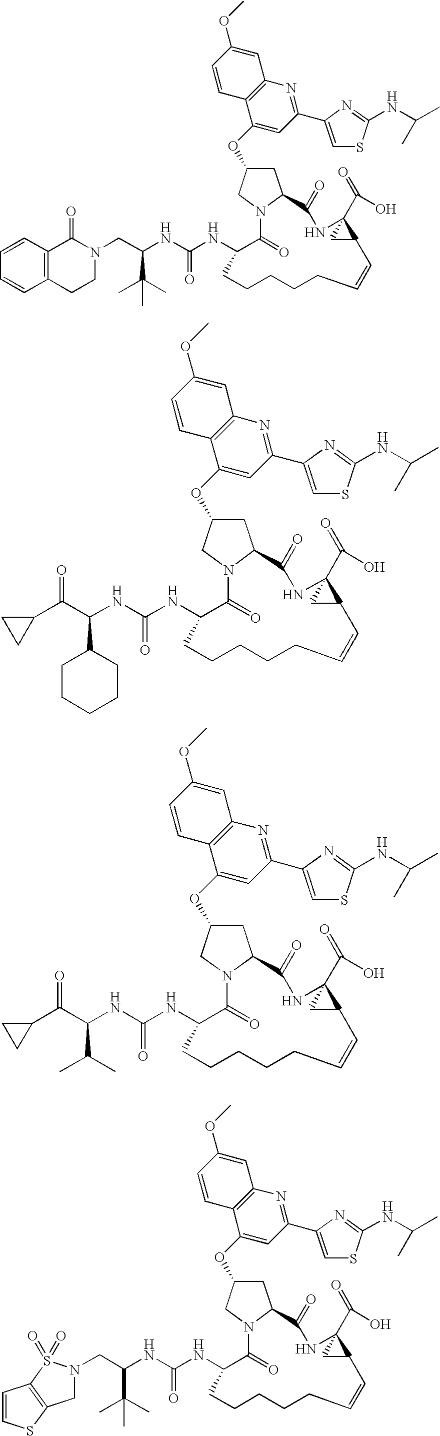 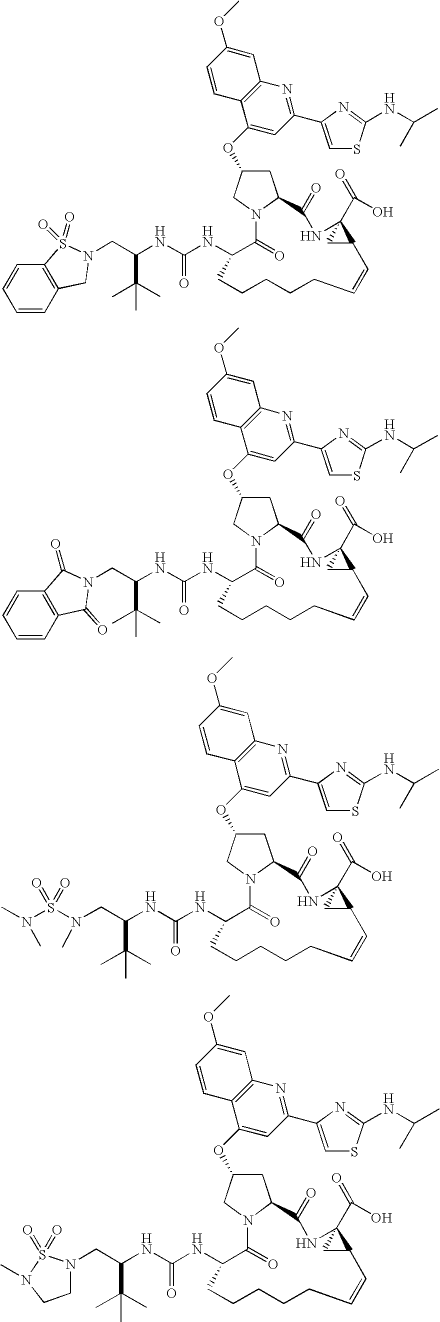 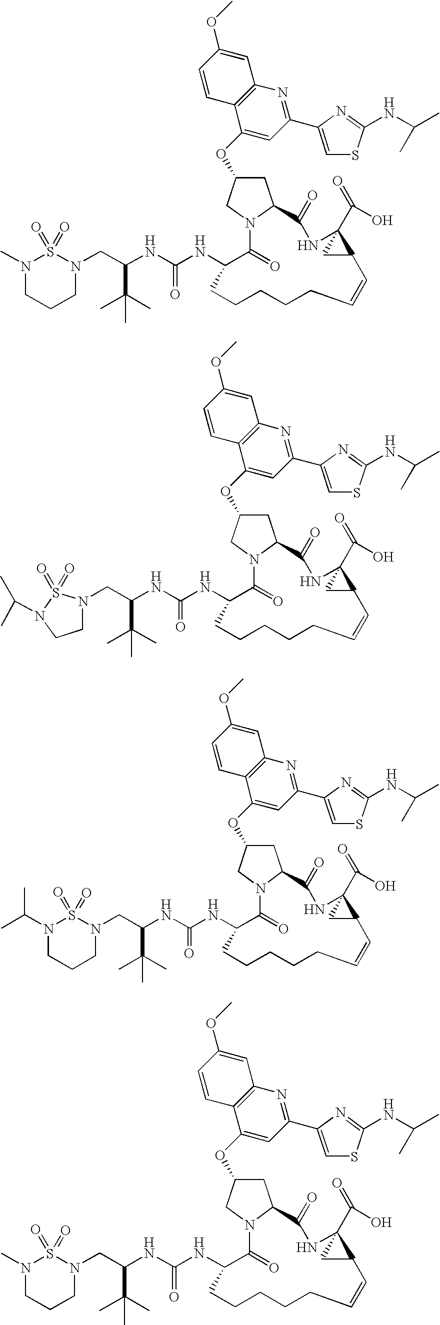 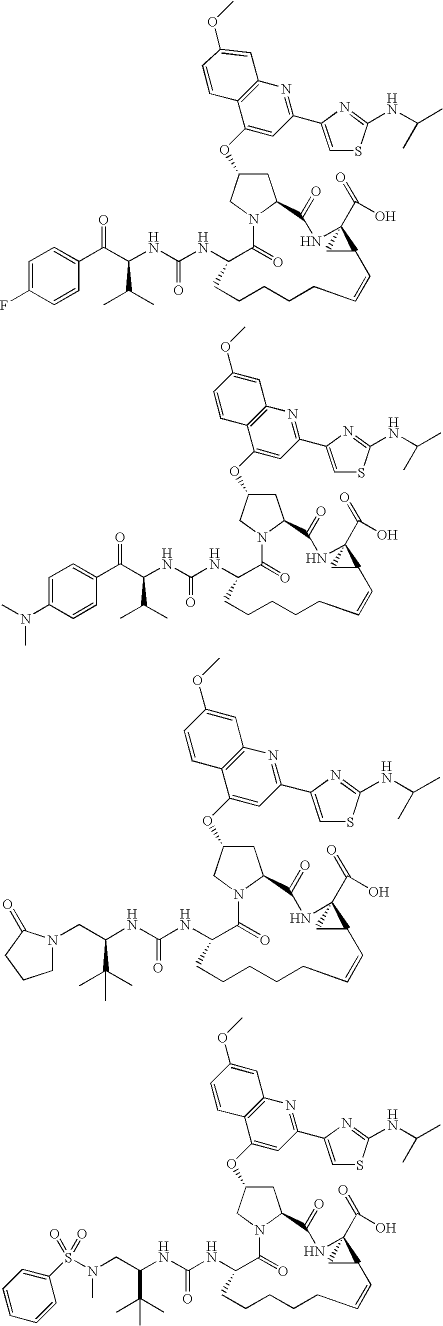 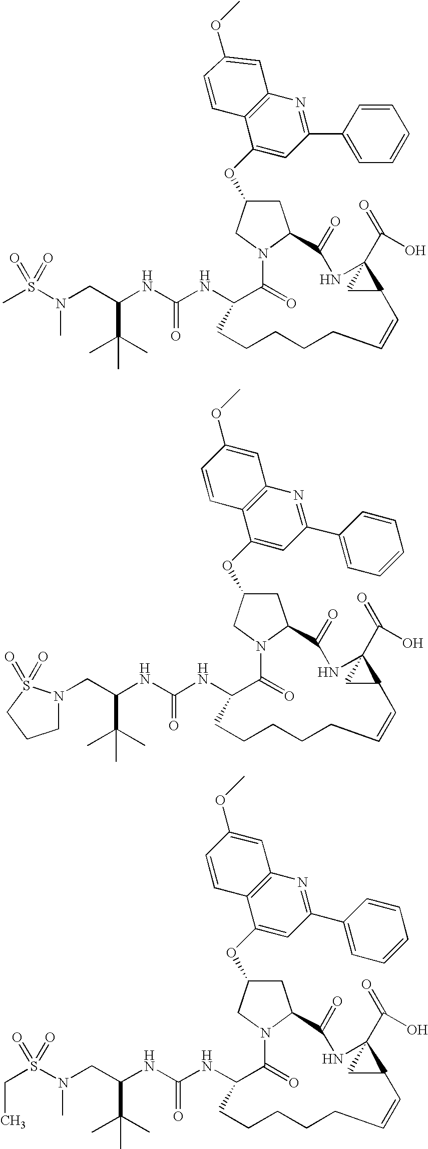 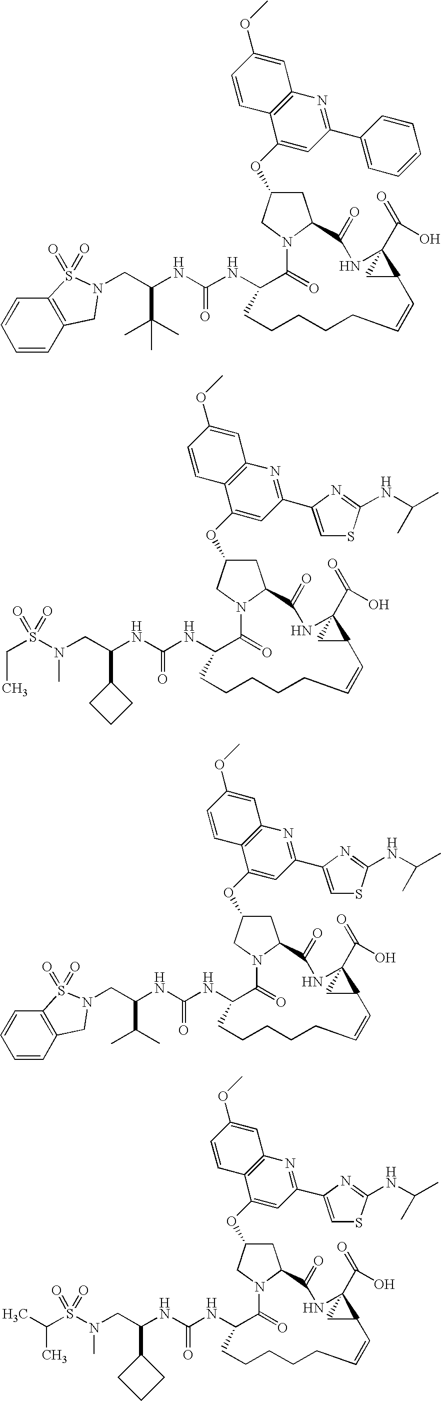 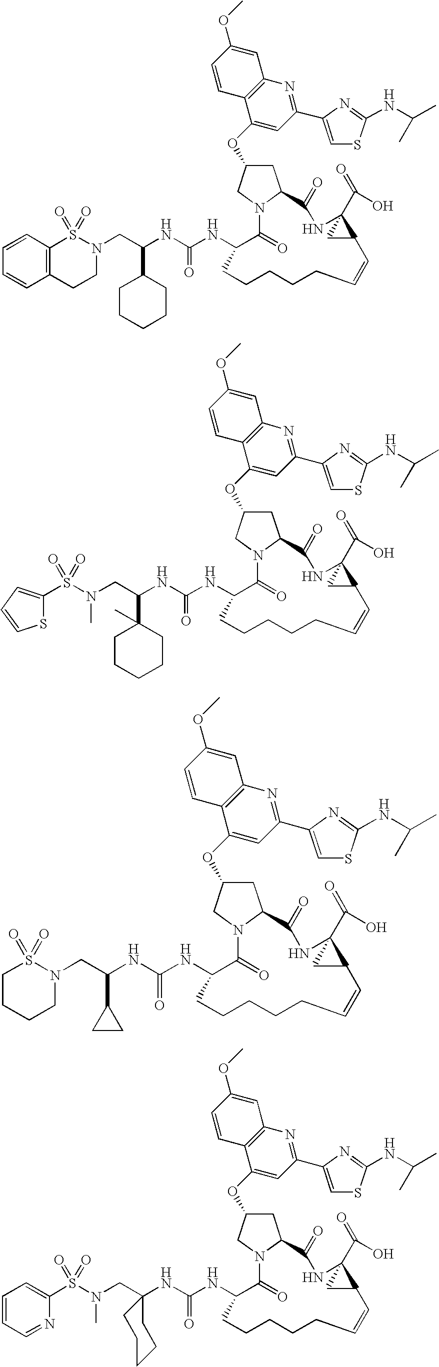 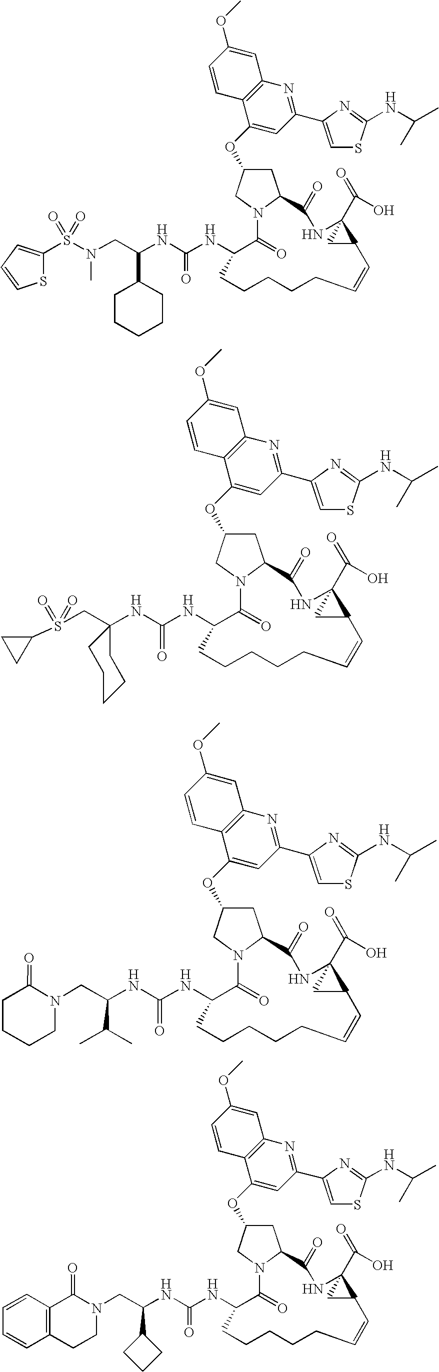 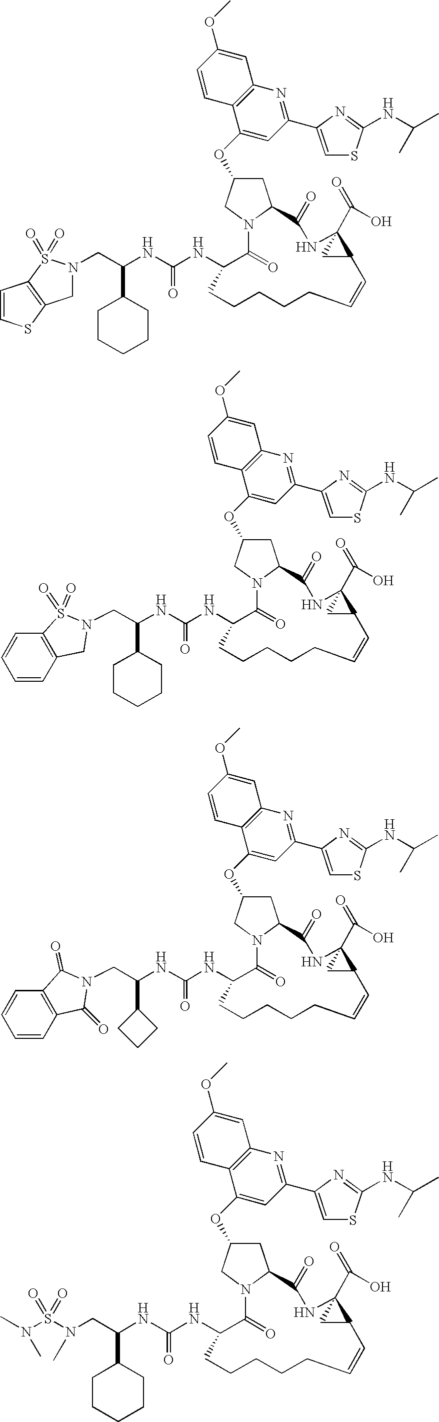 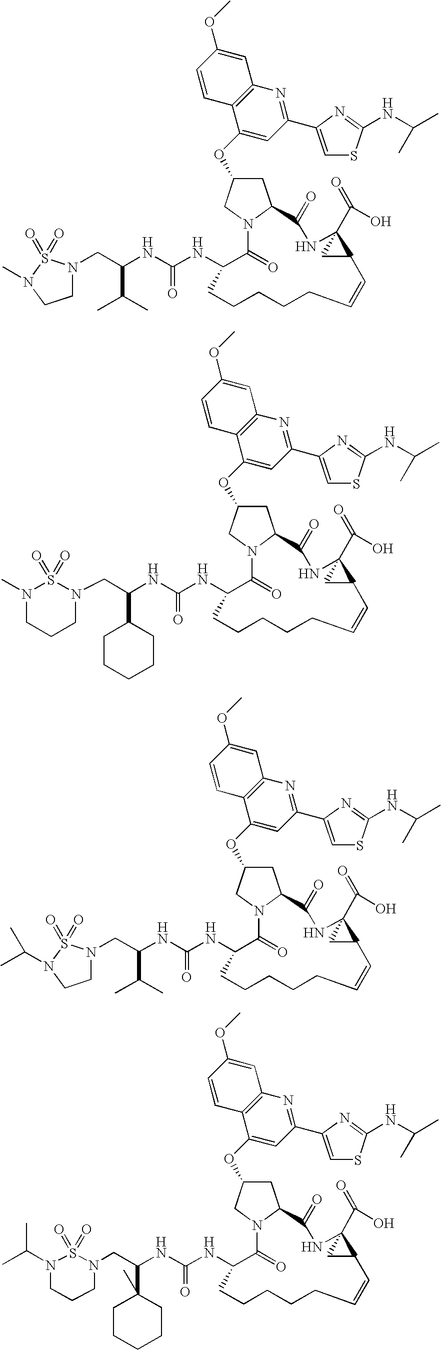 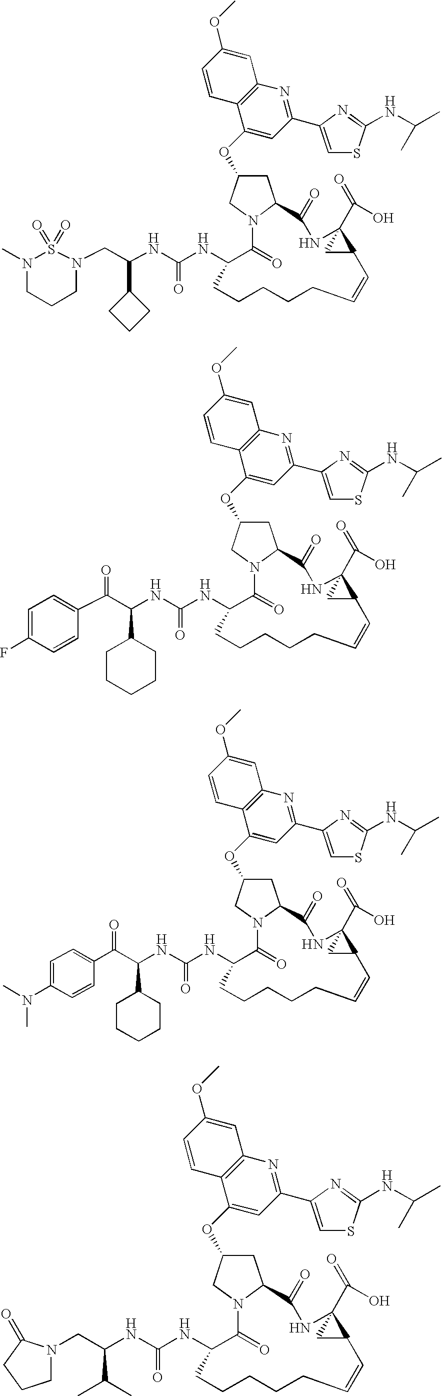 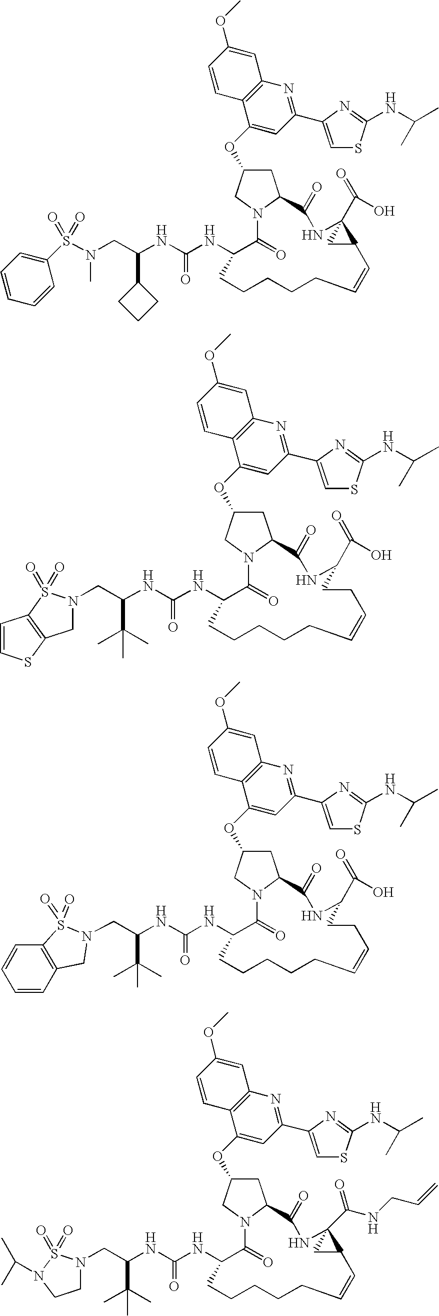 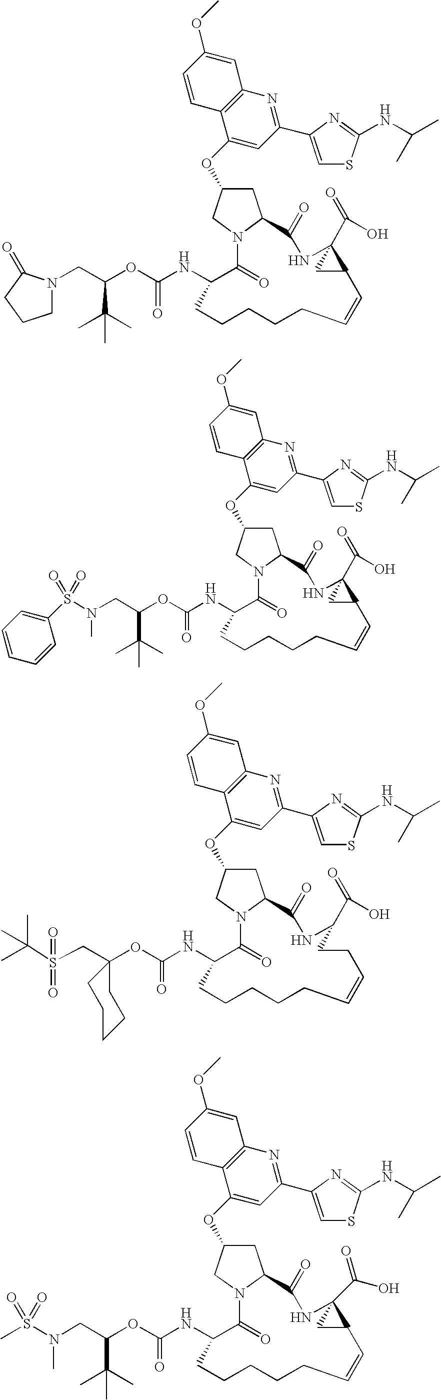 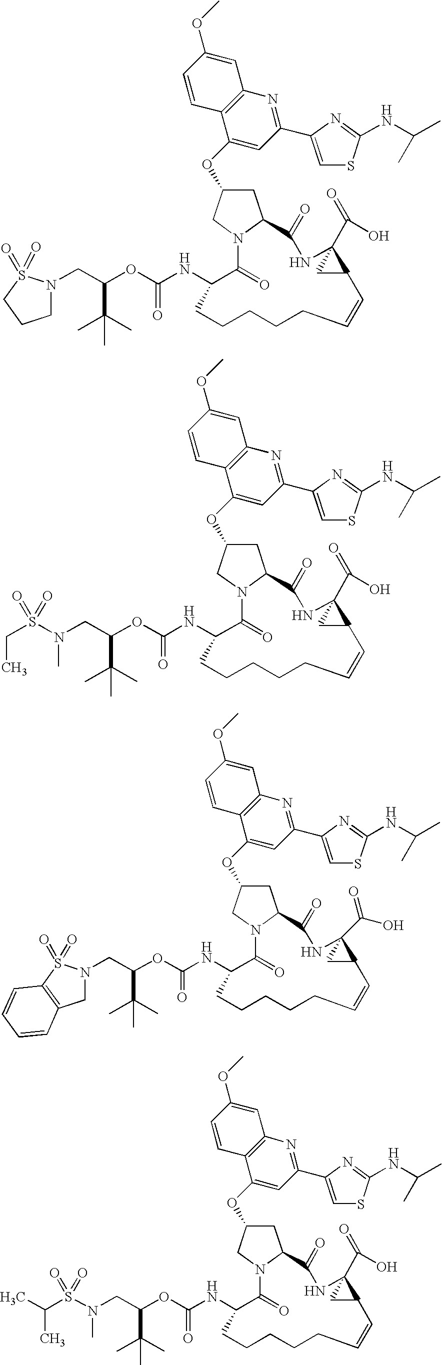 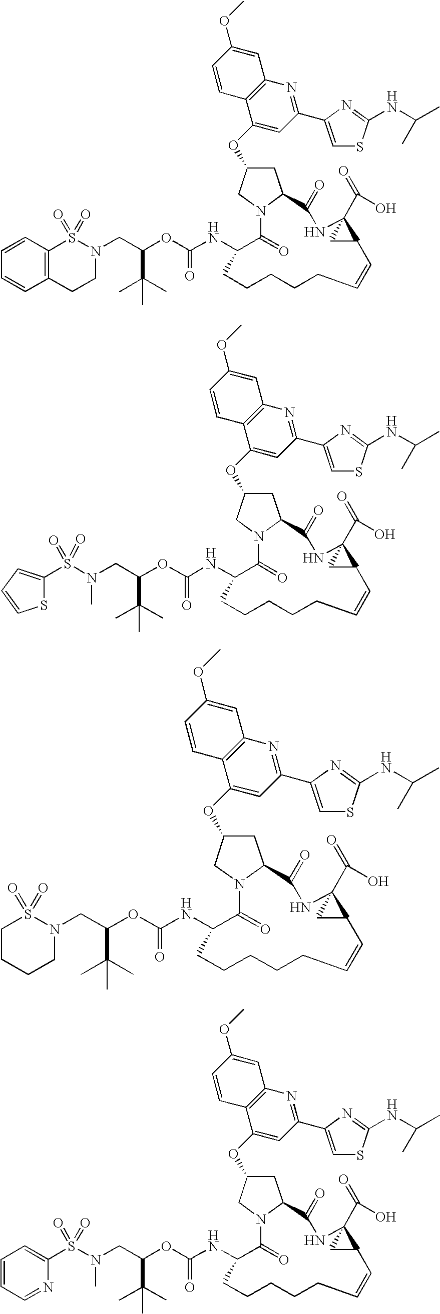 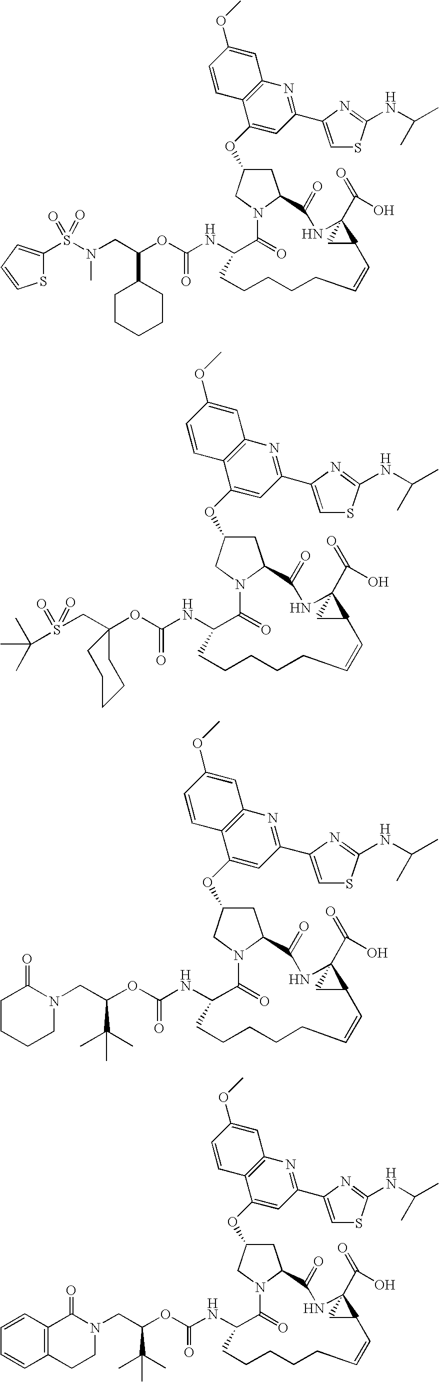 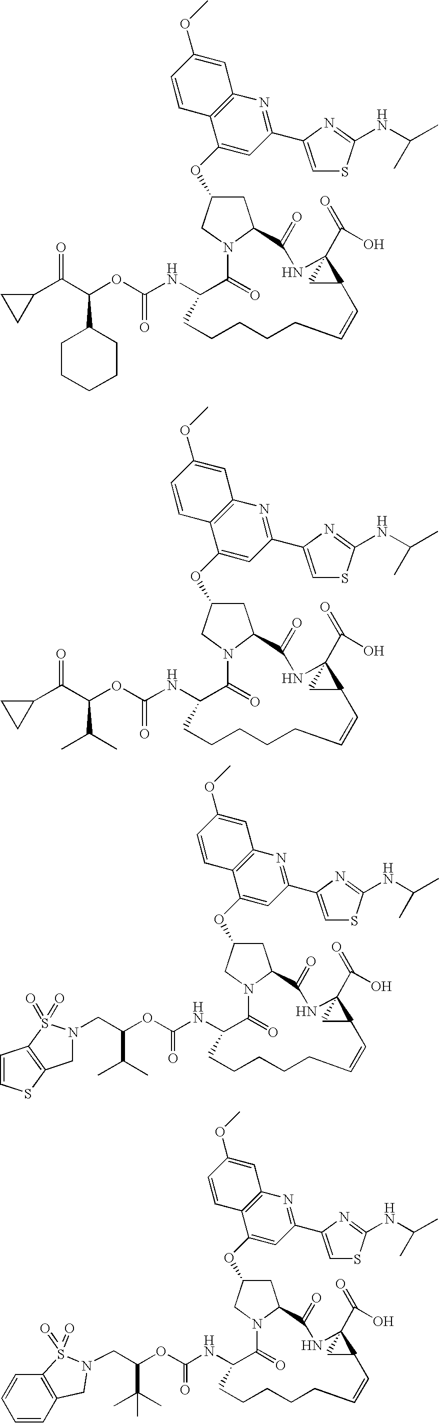 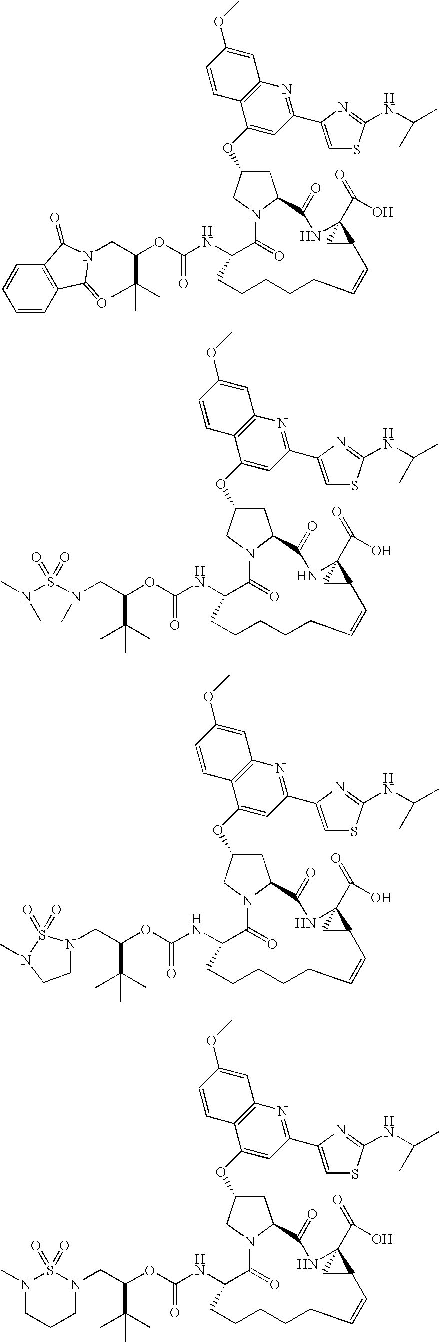 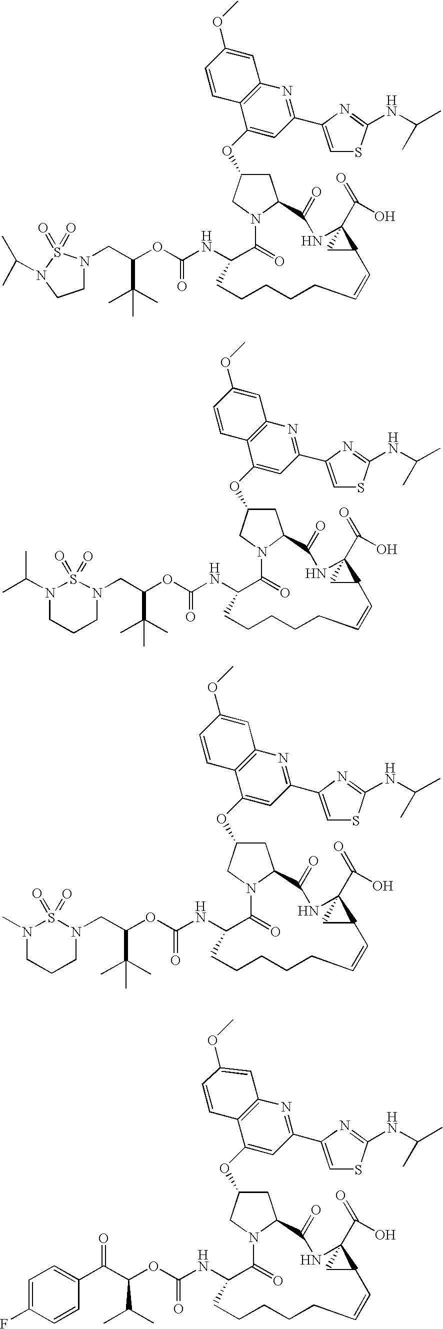 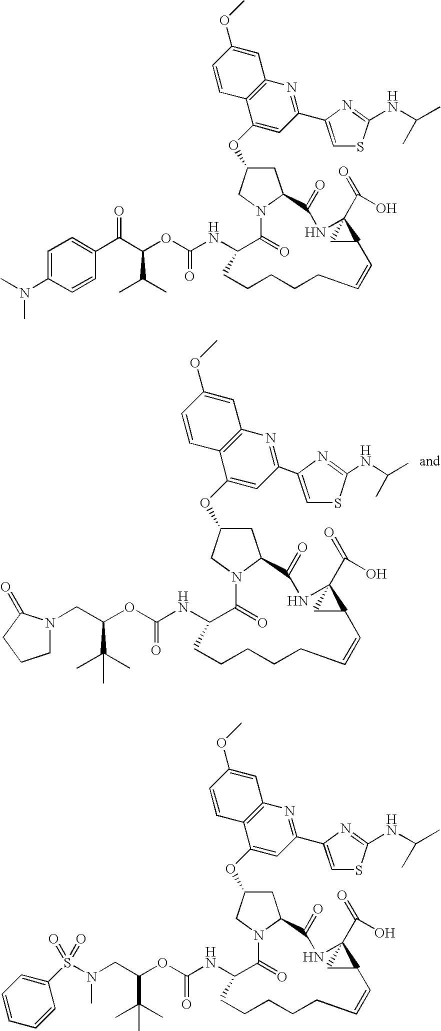
or a pharmaceutically acceptable salt, solvate or ester thereof.

Nonlimiting examples of certain compounds of formula VIII disclosed in U.S. patent application Ser. No. 10/993,394 are: 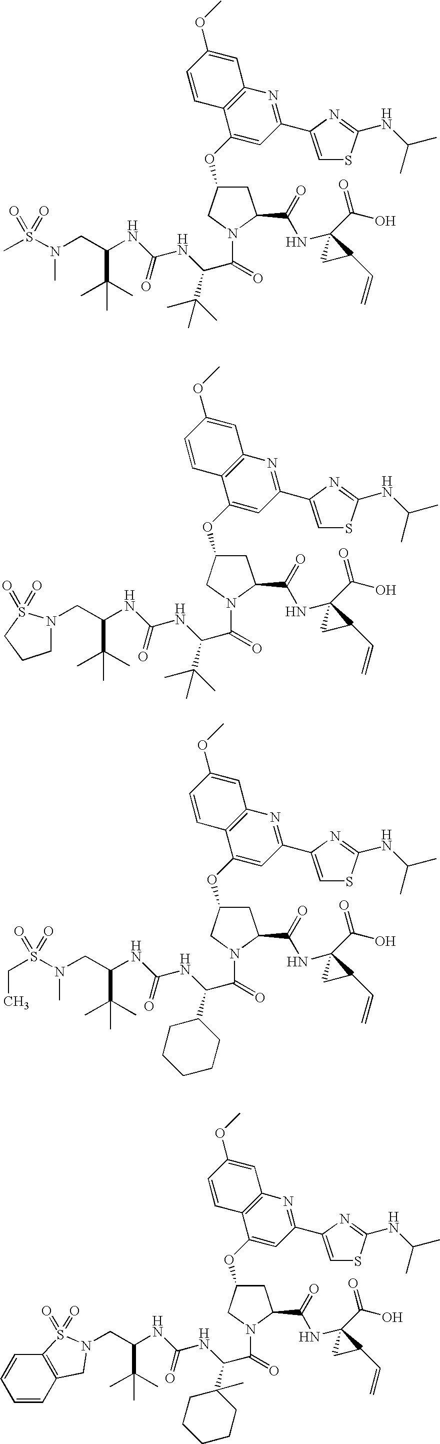 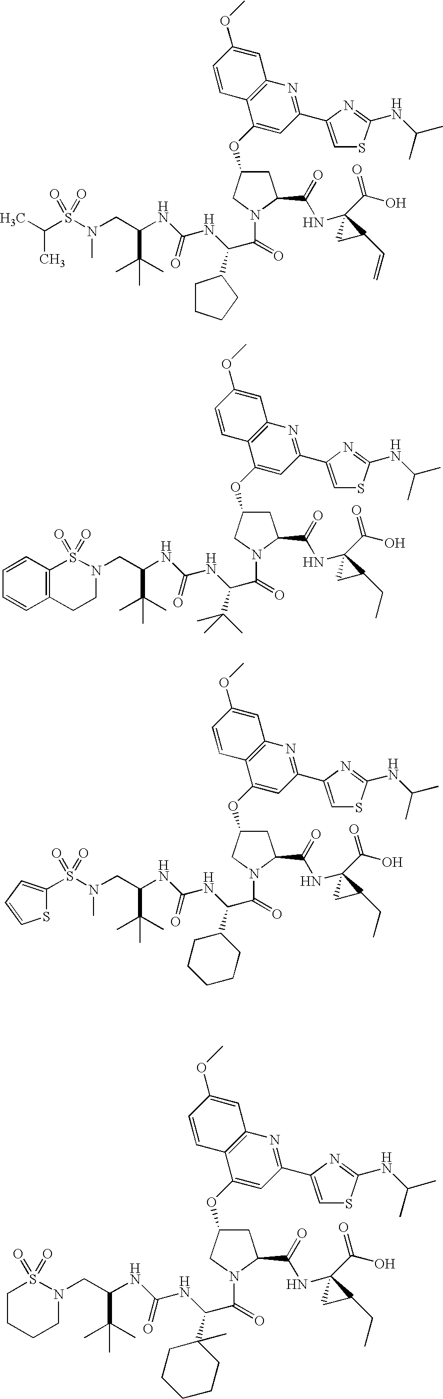 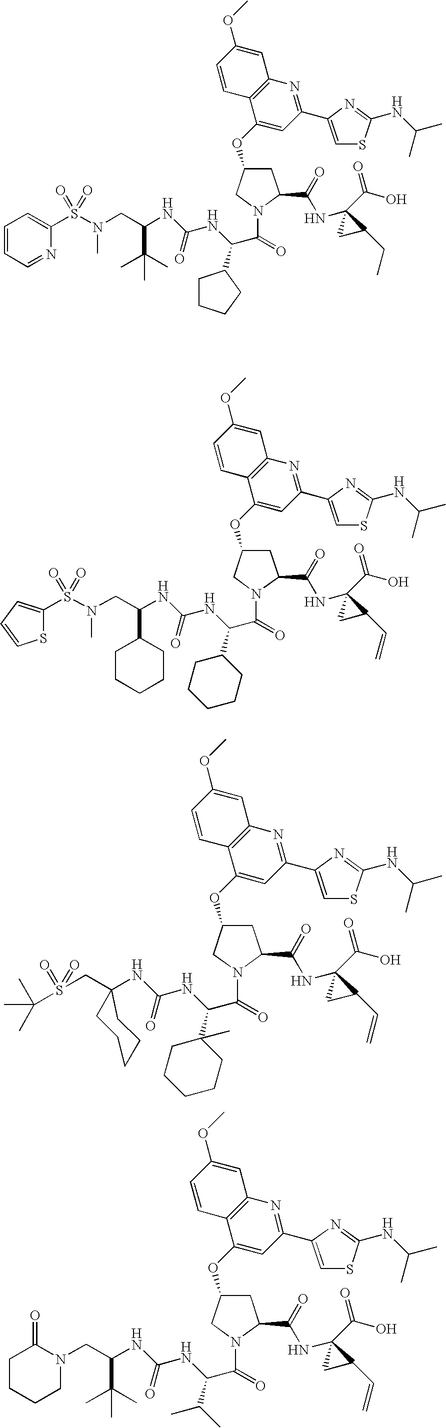 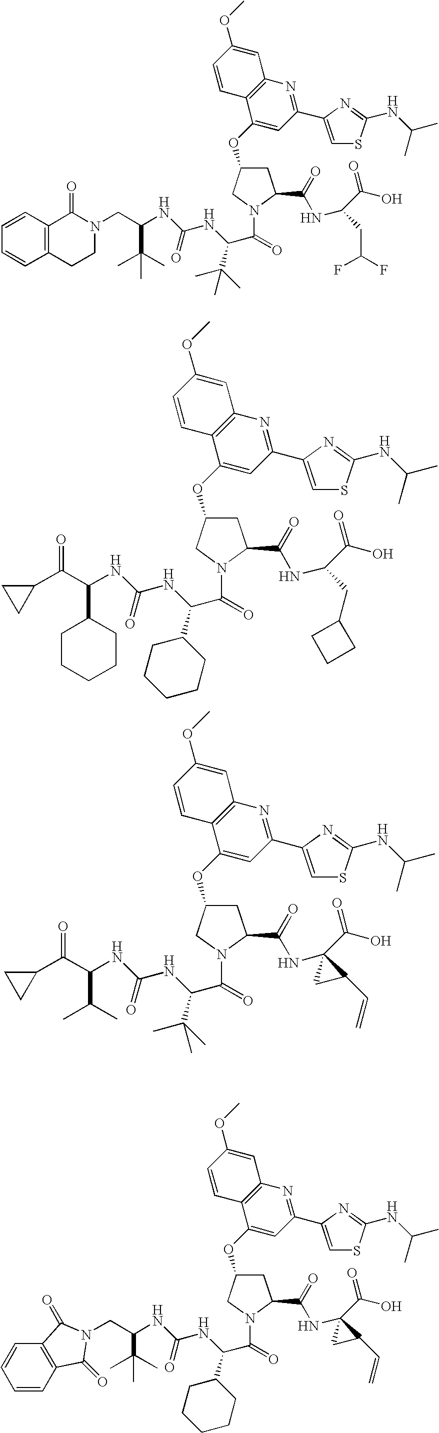 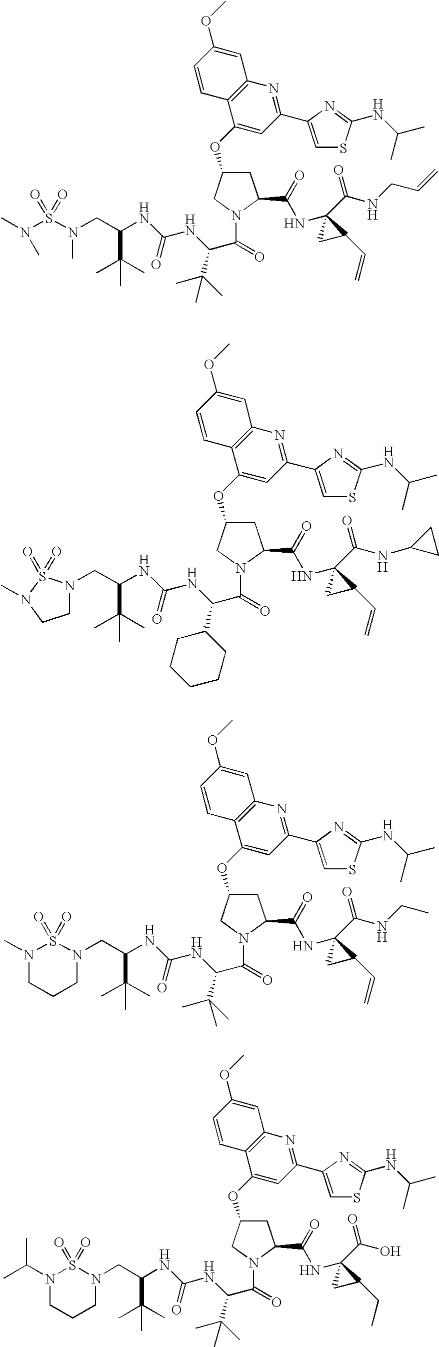 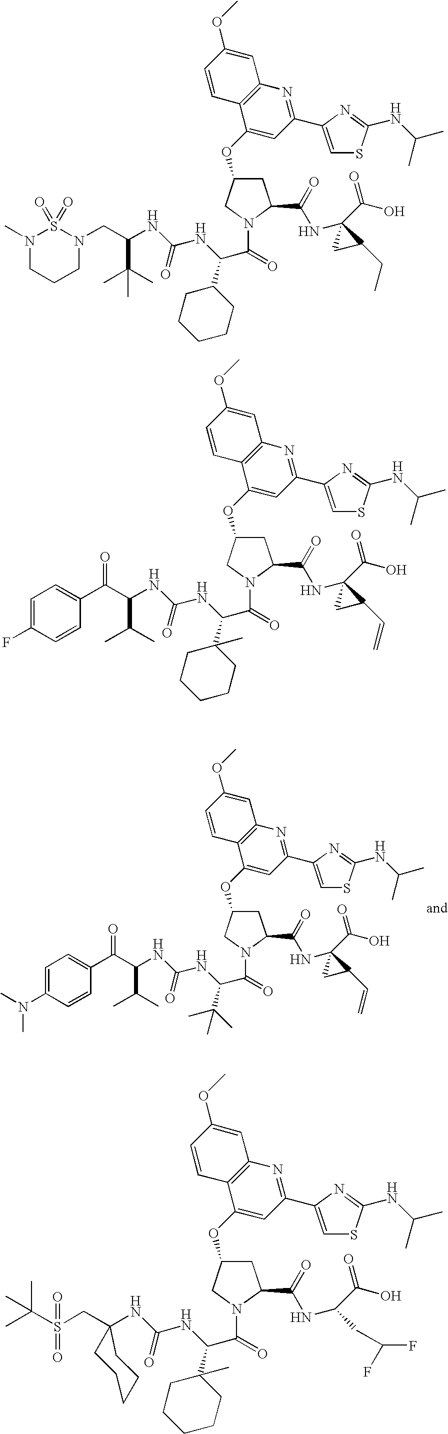
or a pharmaceutically acceptable salt, solvate or ester thereof.

Nonlimiting examples of certain compounds of formula IX disclosed in U.S. patent application Ser. No. 10/993,394 are: 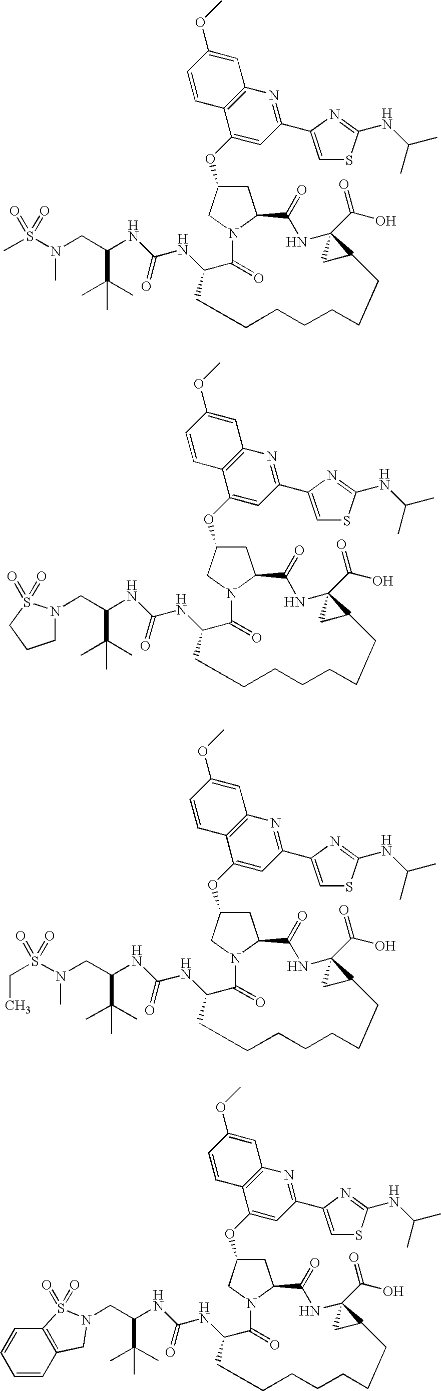 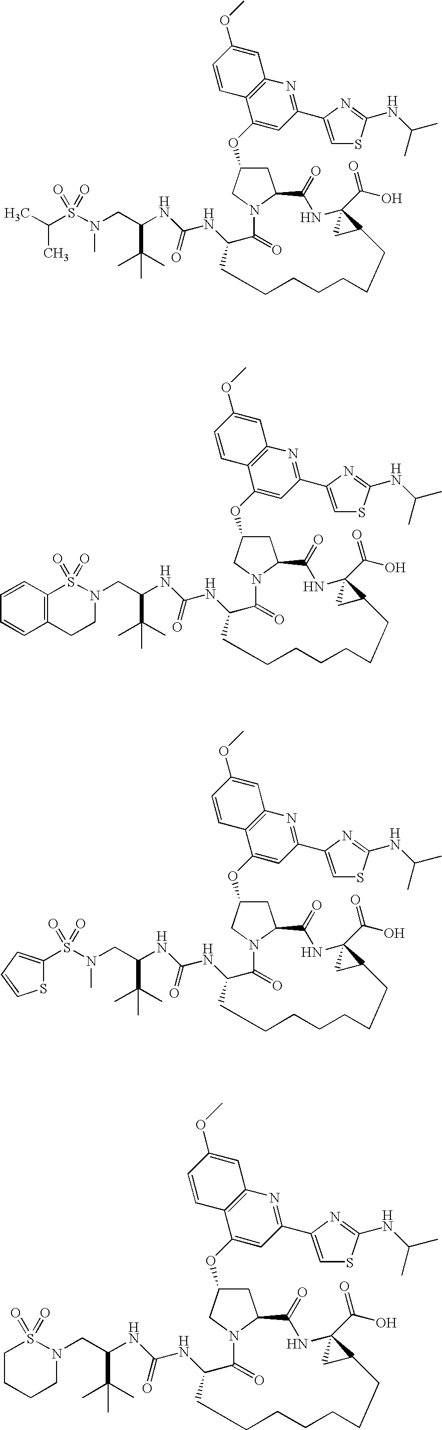 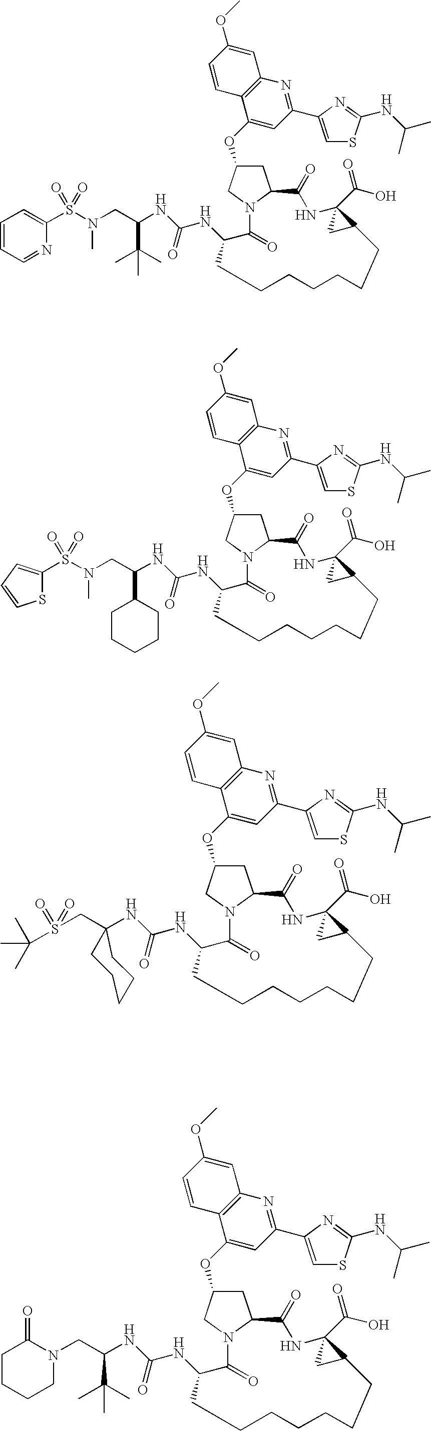 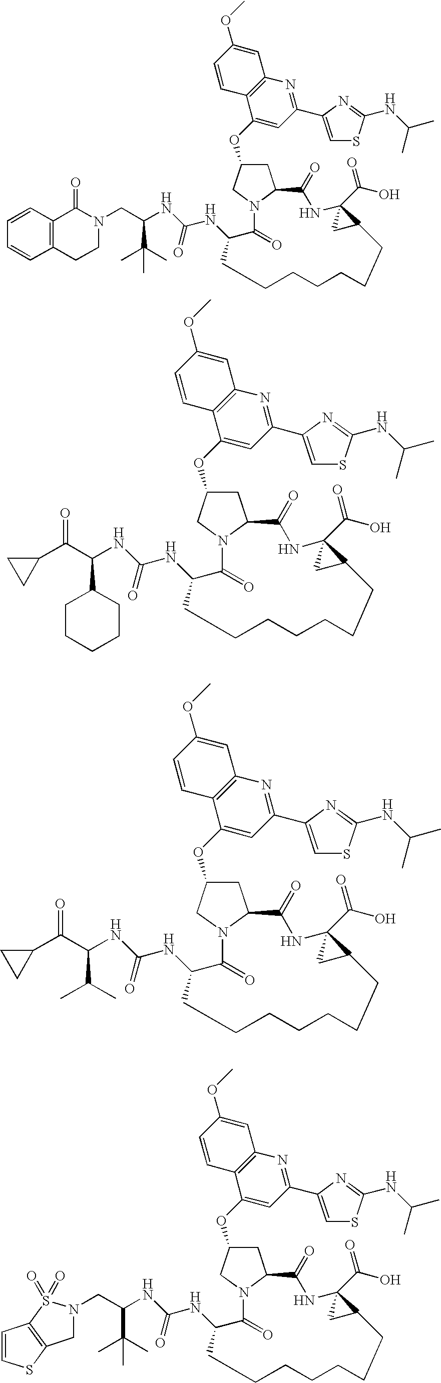 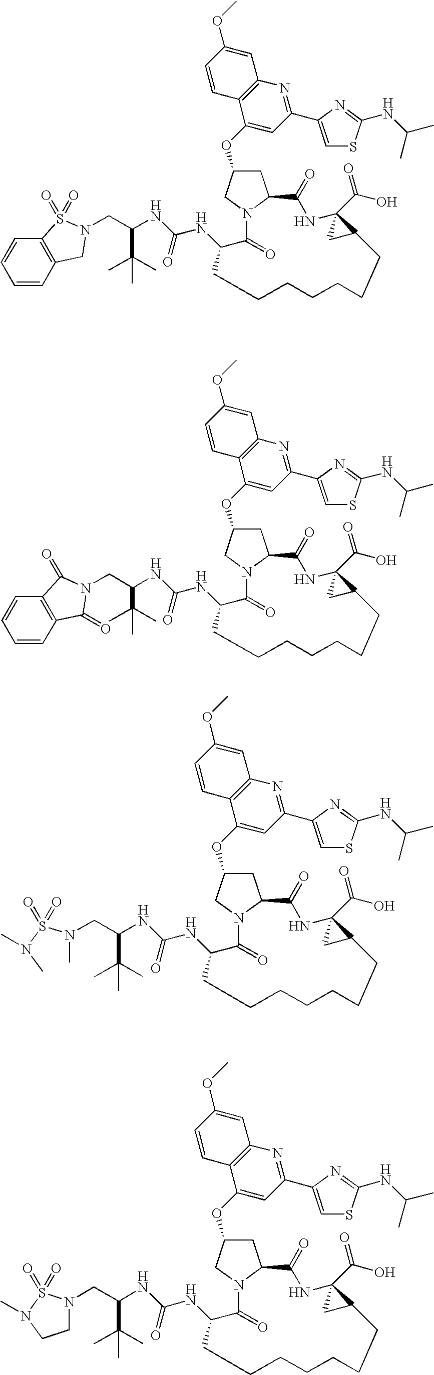 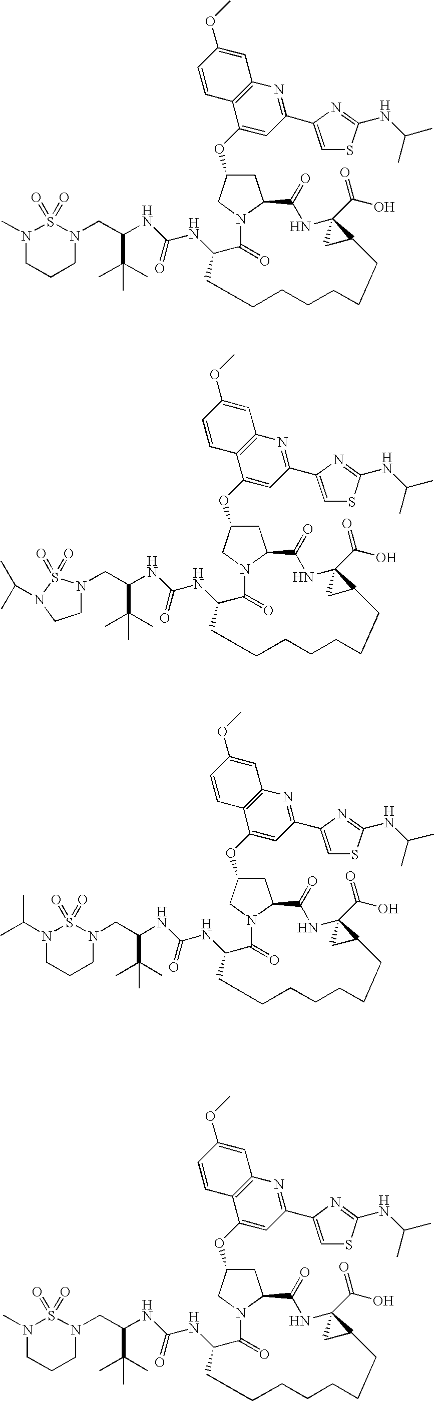 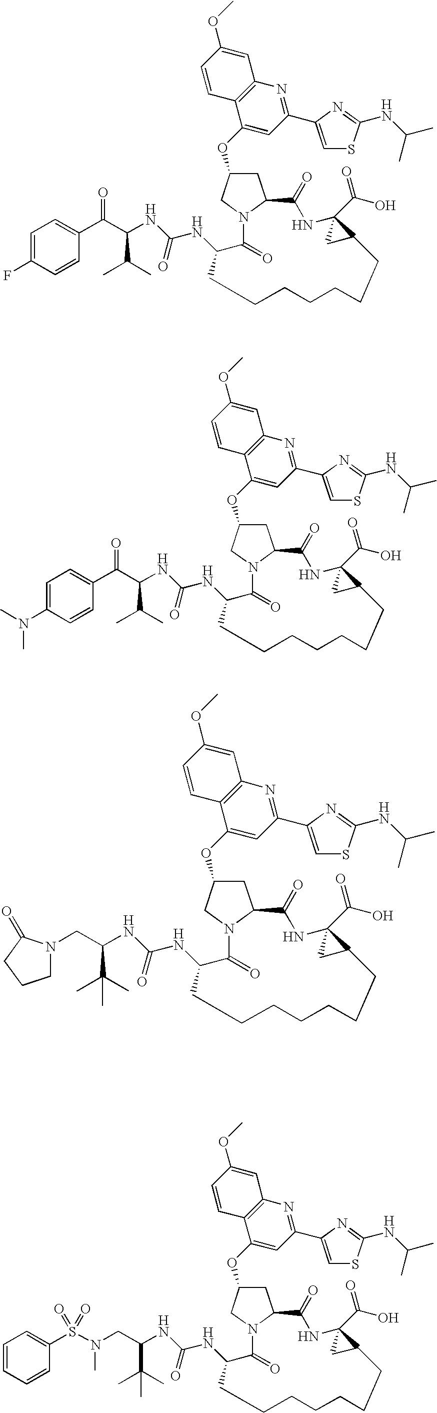 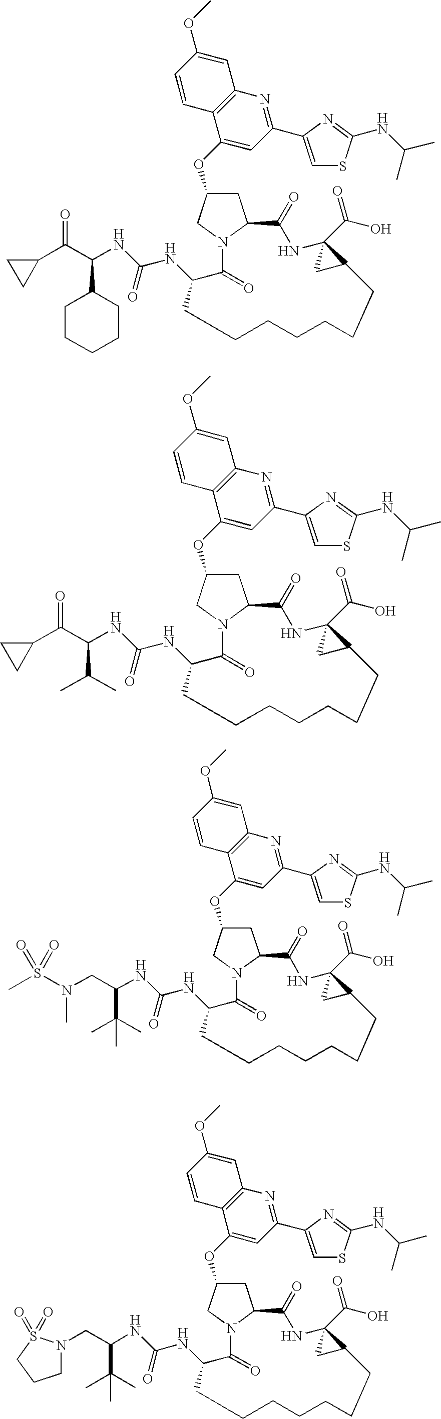 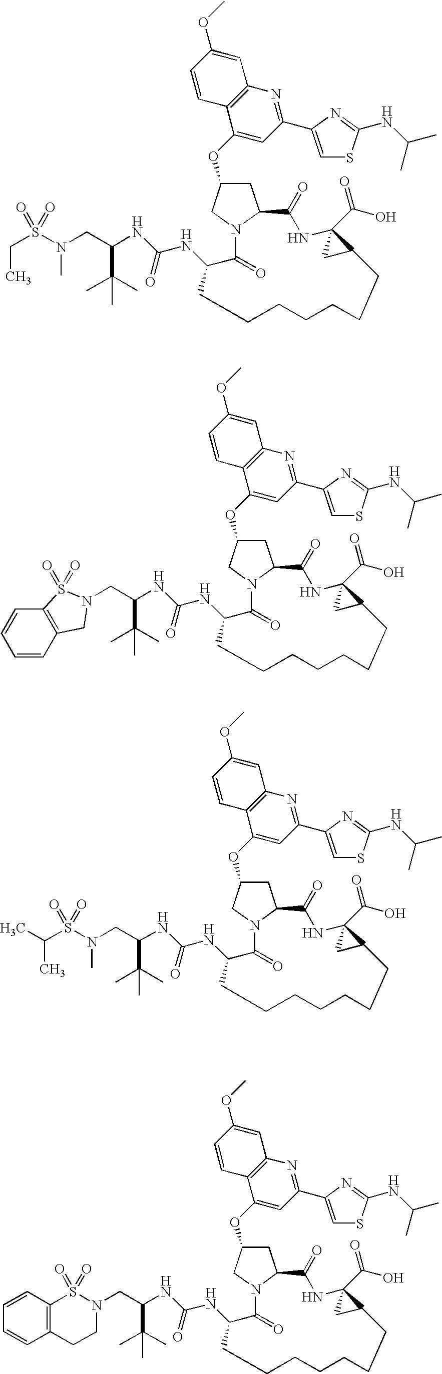 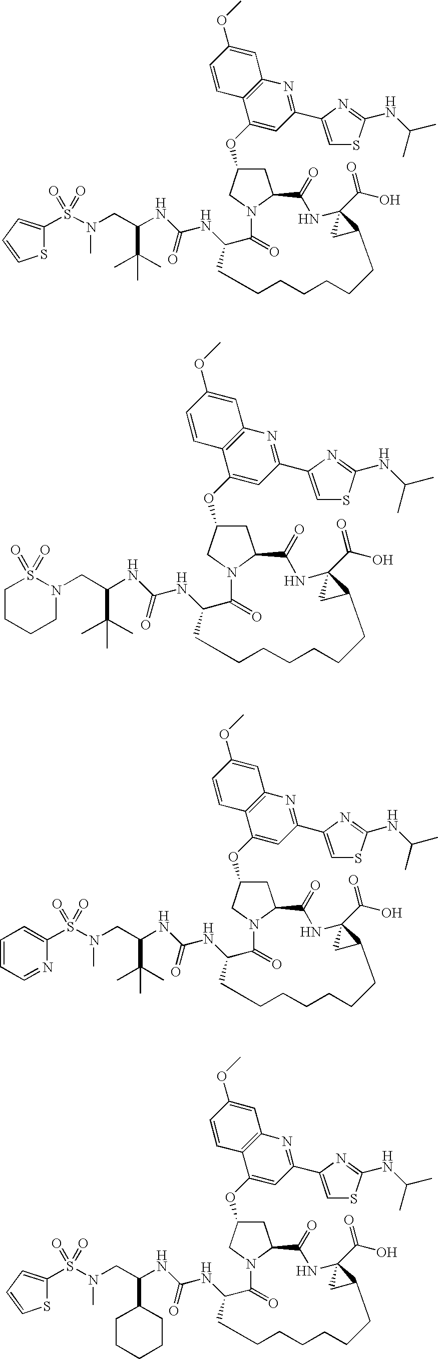 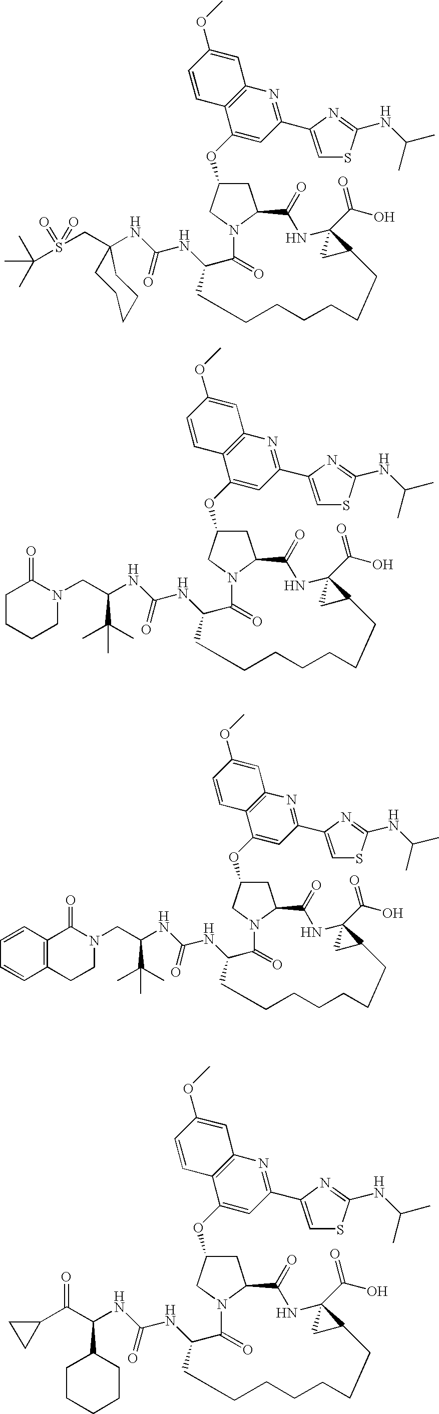 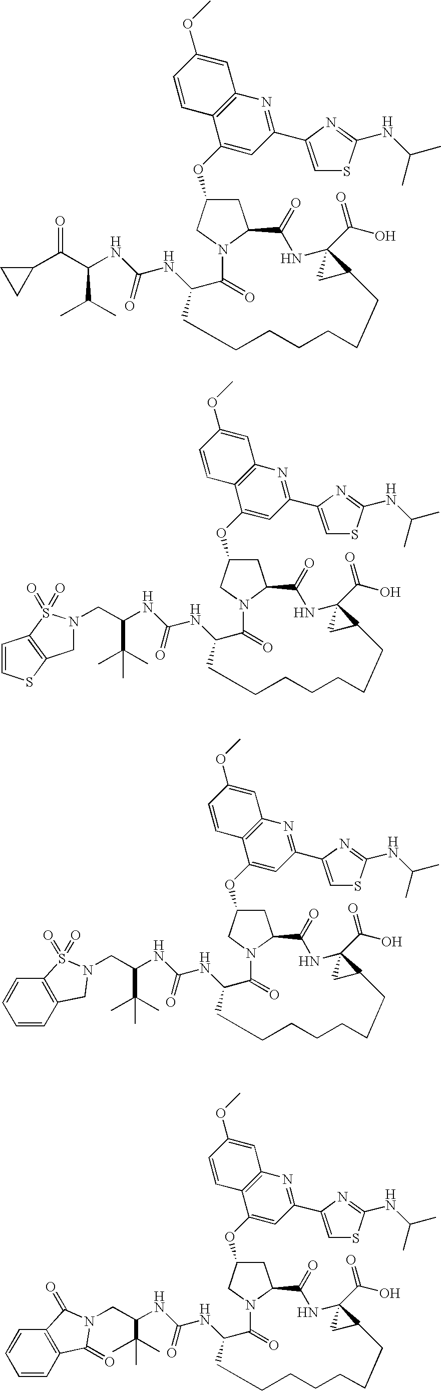 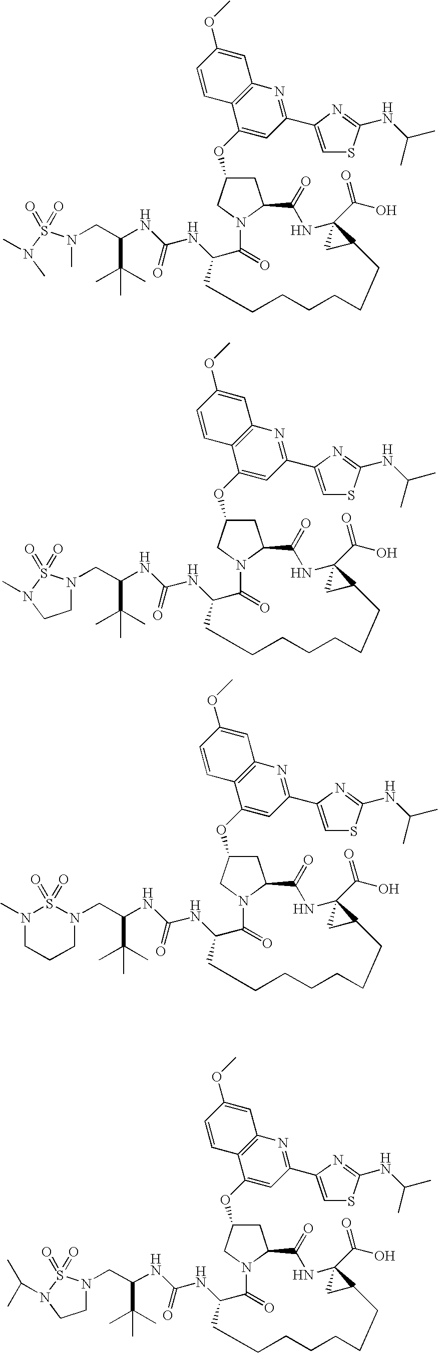 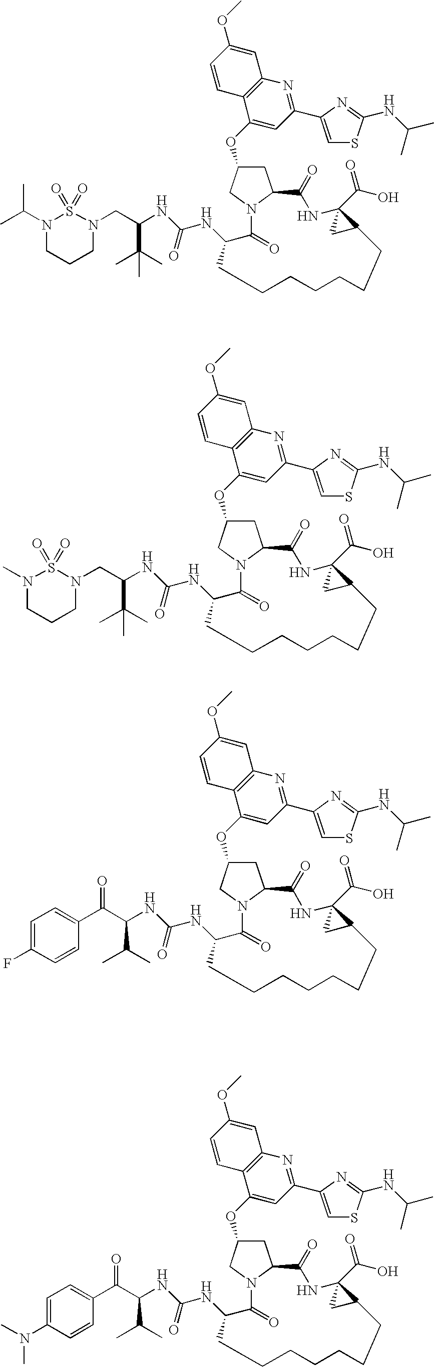 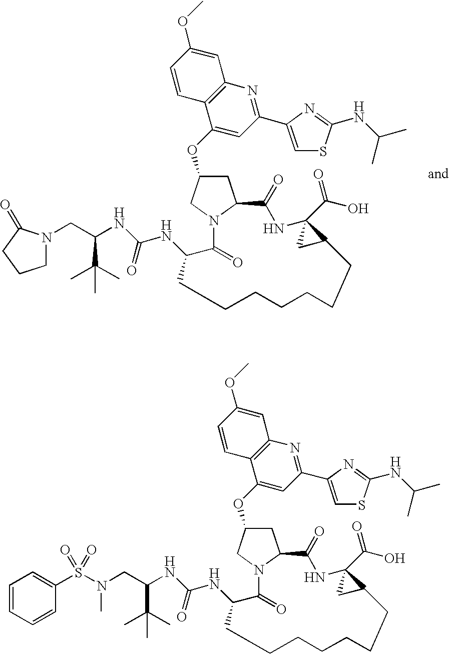
or a pharmaceutically acceptable salt, solvate or ester thereof.

Compounds of formula X are disclosed in U.S. patent application Ser. No. 11/065,572 filed Feb. 24, 2005 and the preparation of the compounds are detailed in the experimental section of this application set forth hereinbelow.

Non-limiting examples of certain compounds disclosed in U.S. patent application Ser. No. 11/065,572 filed Feb. 24, 2005 are:

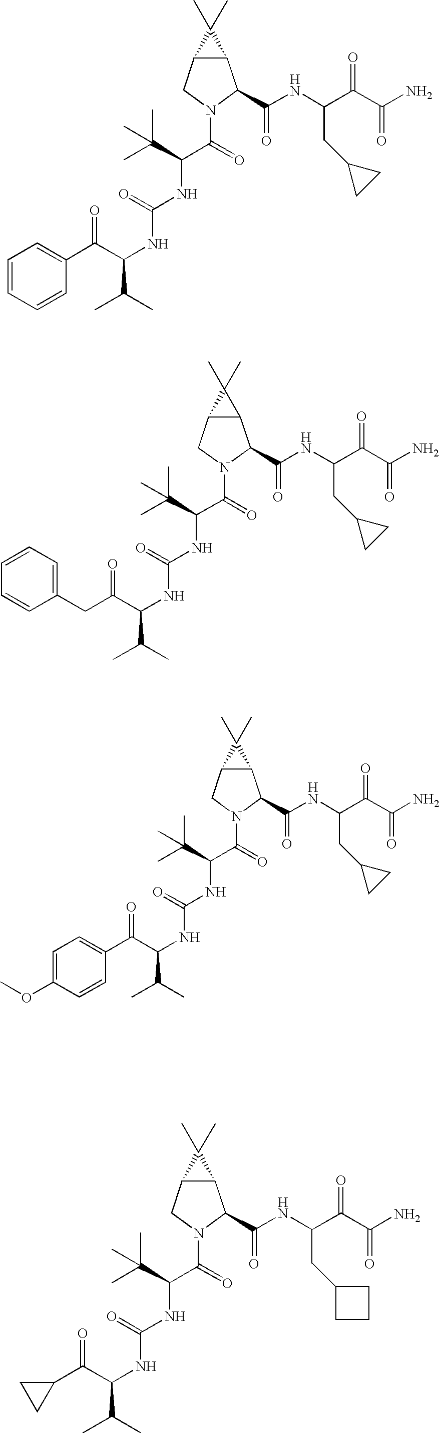

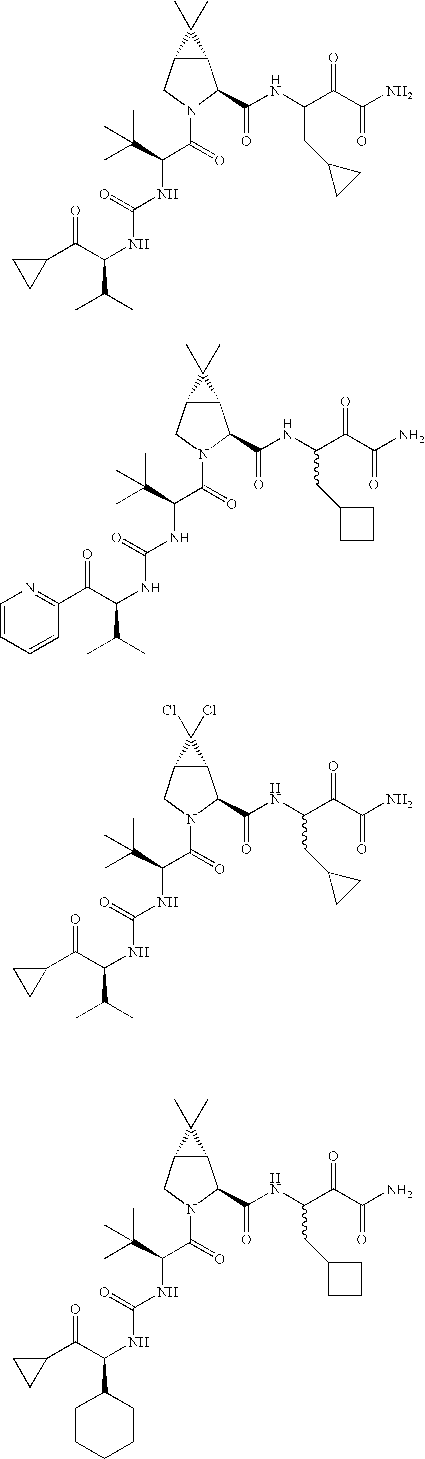

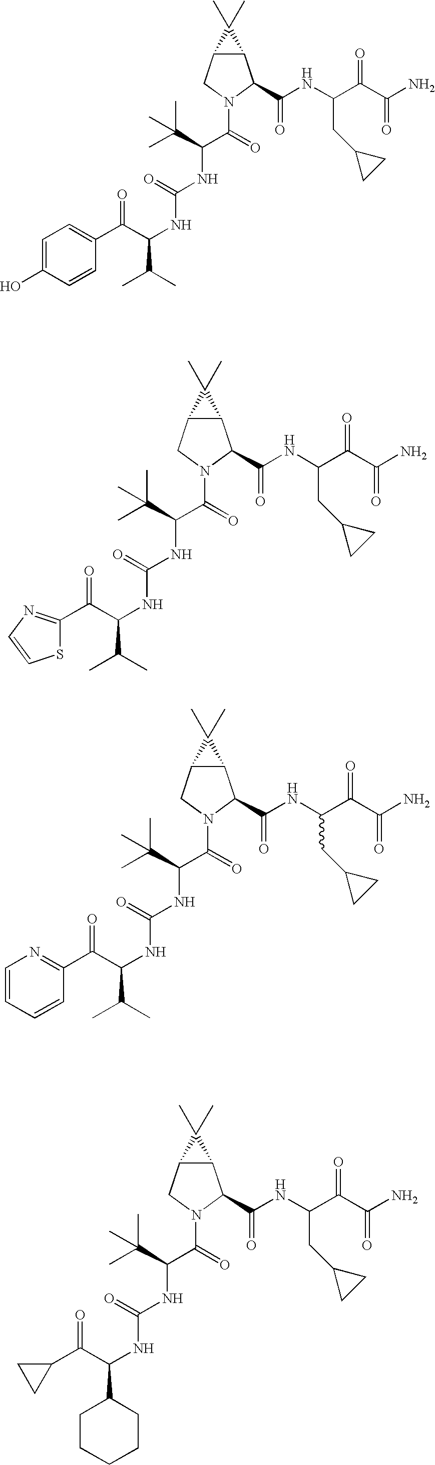

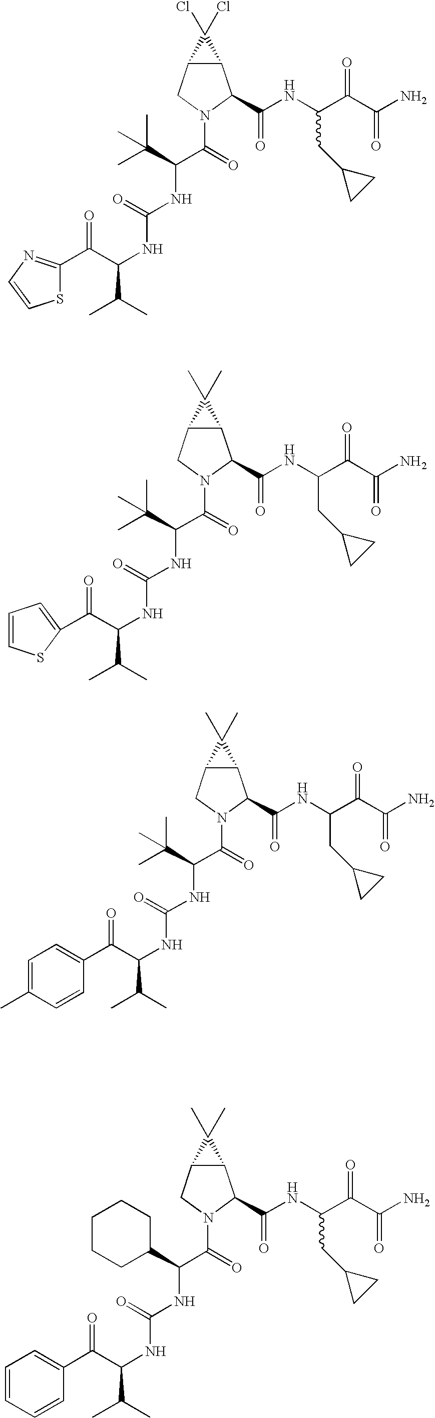

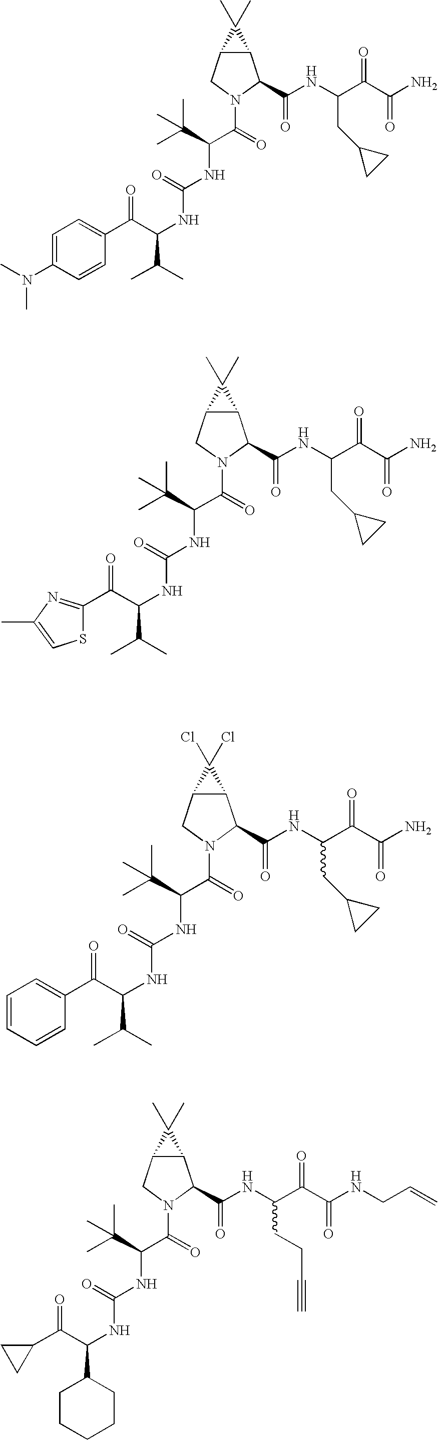

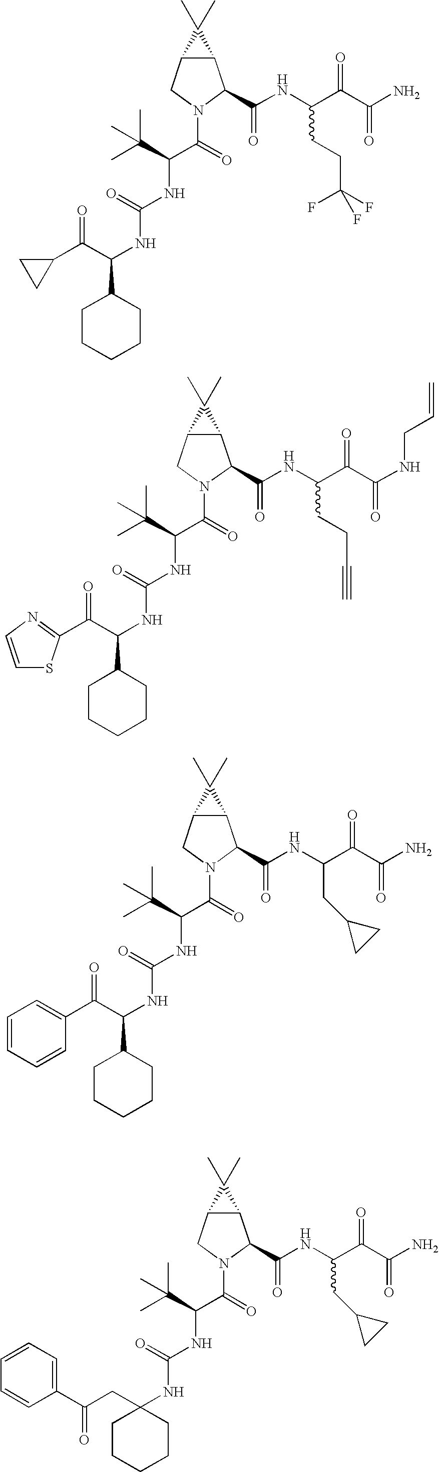

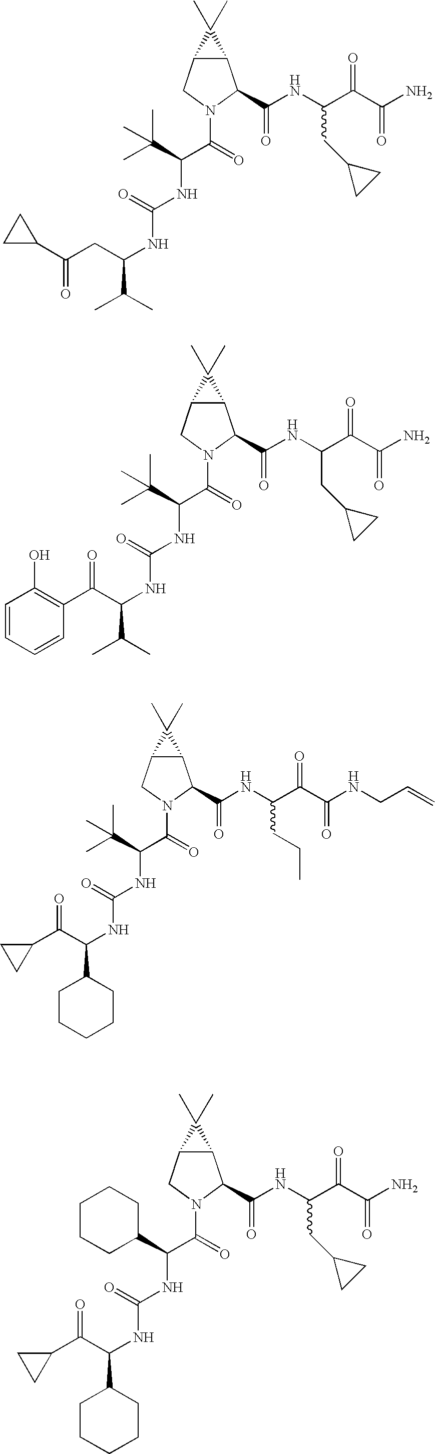

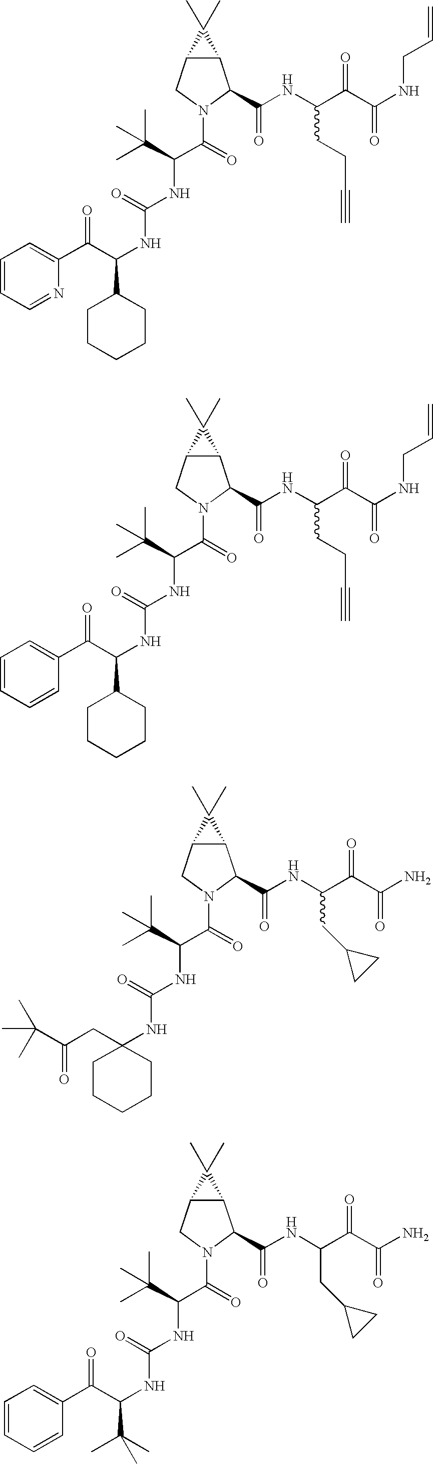

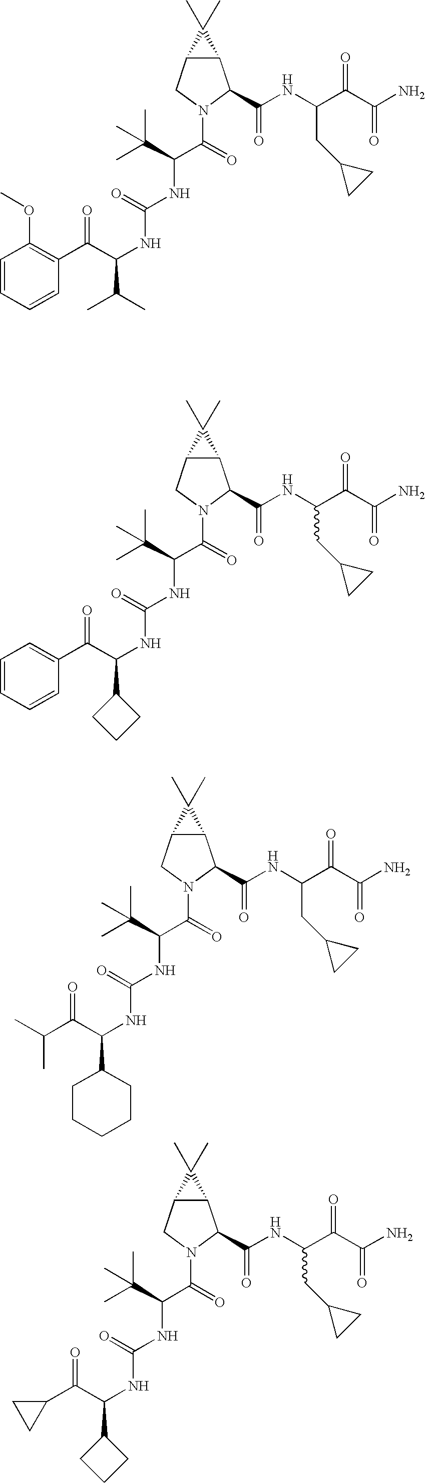

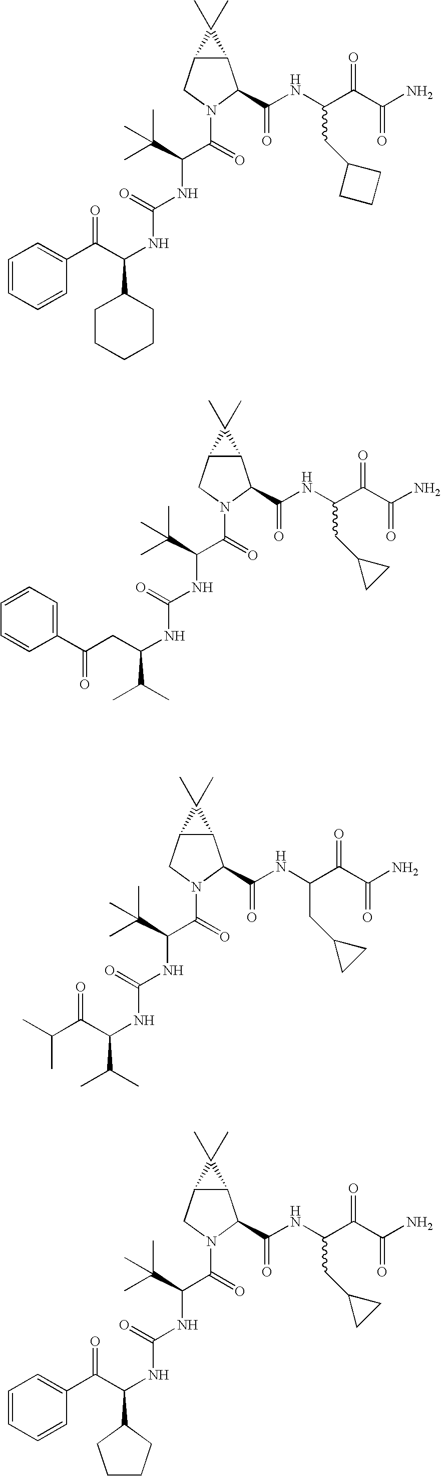

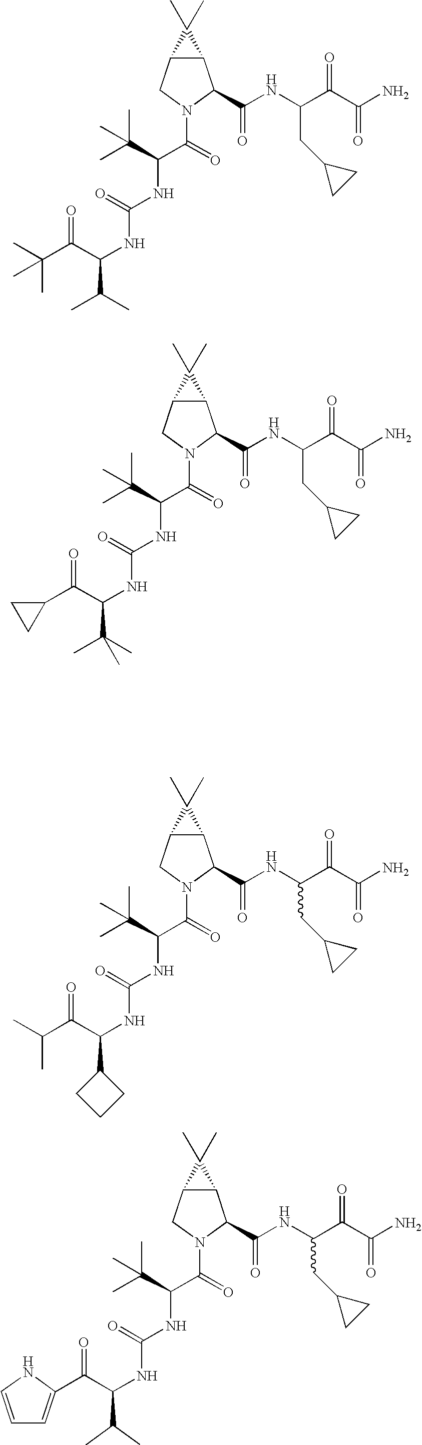

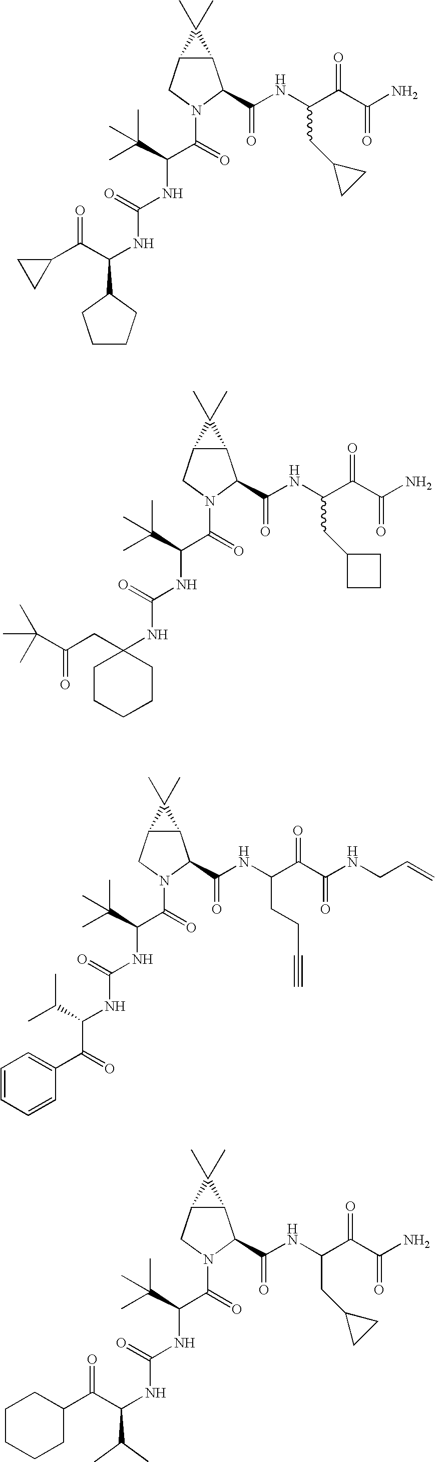

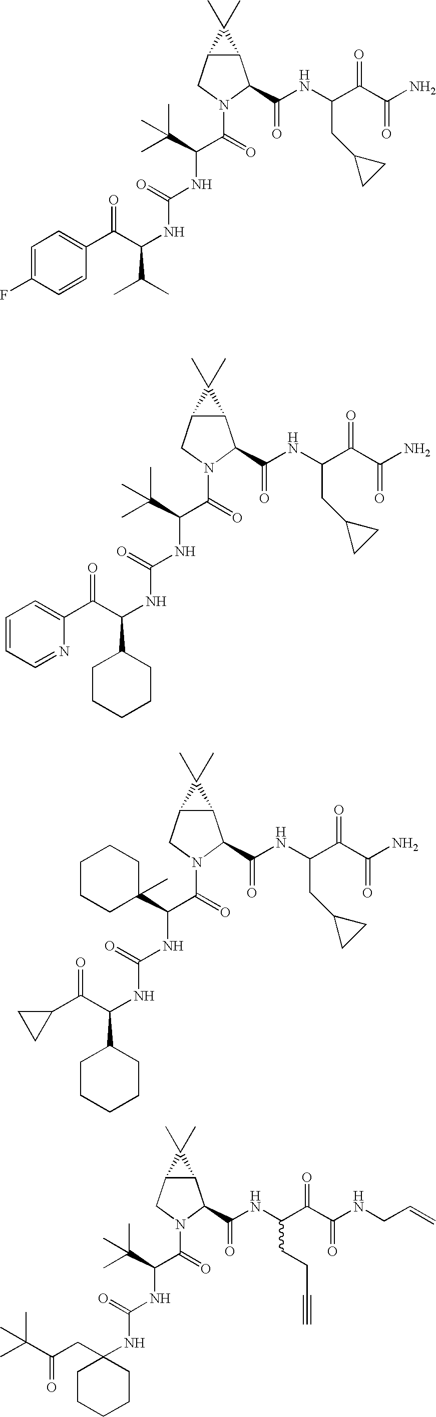

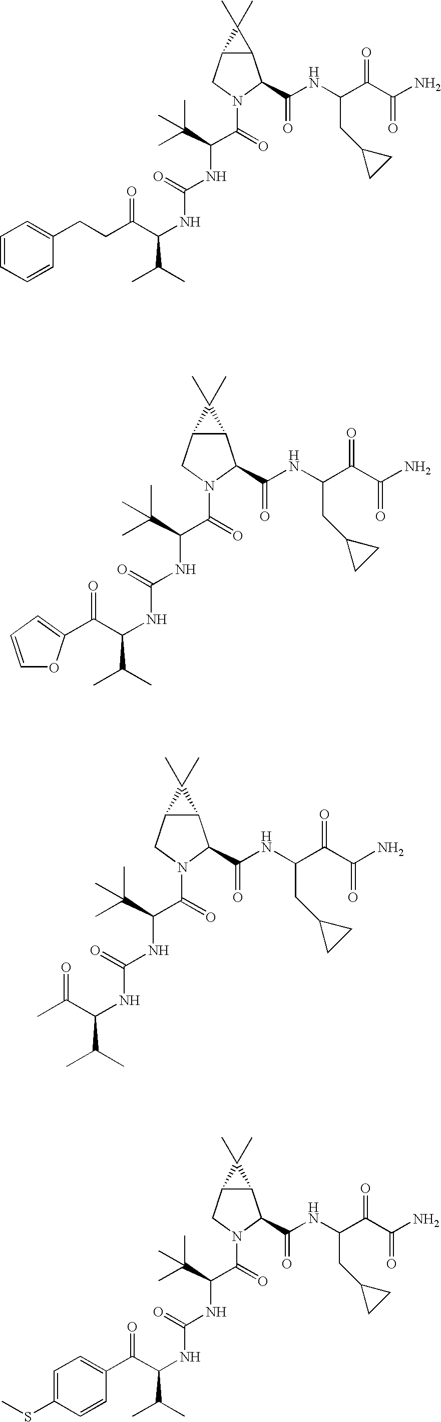

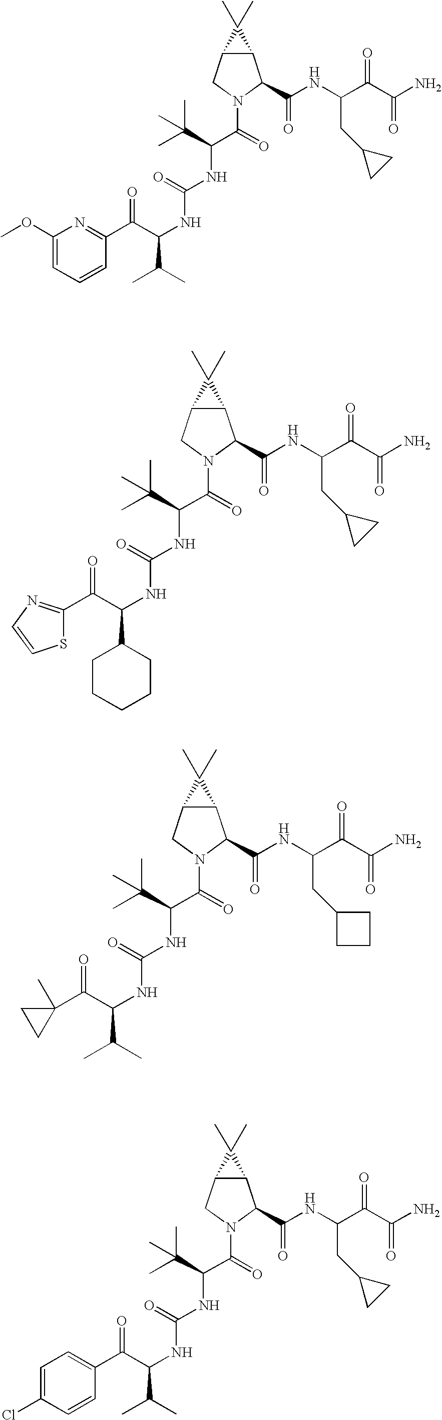

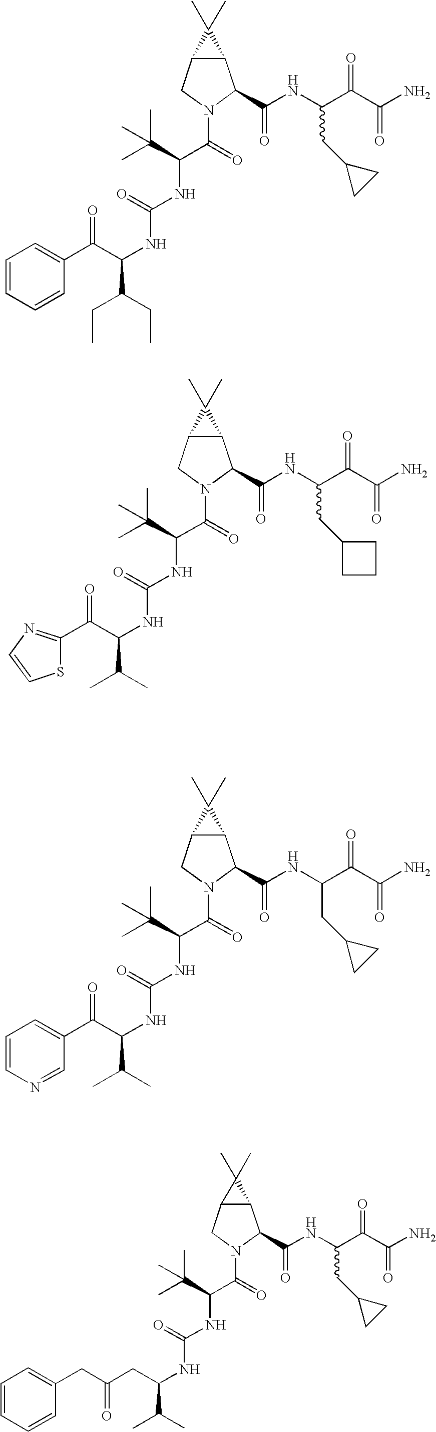

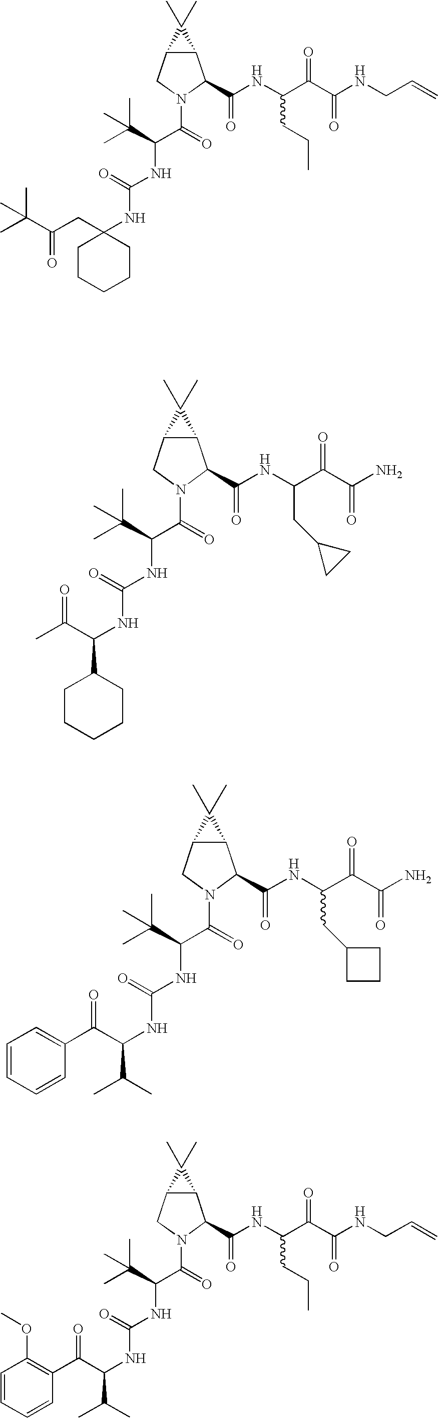

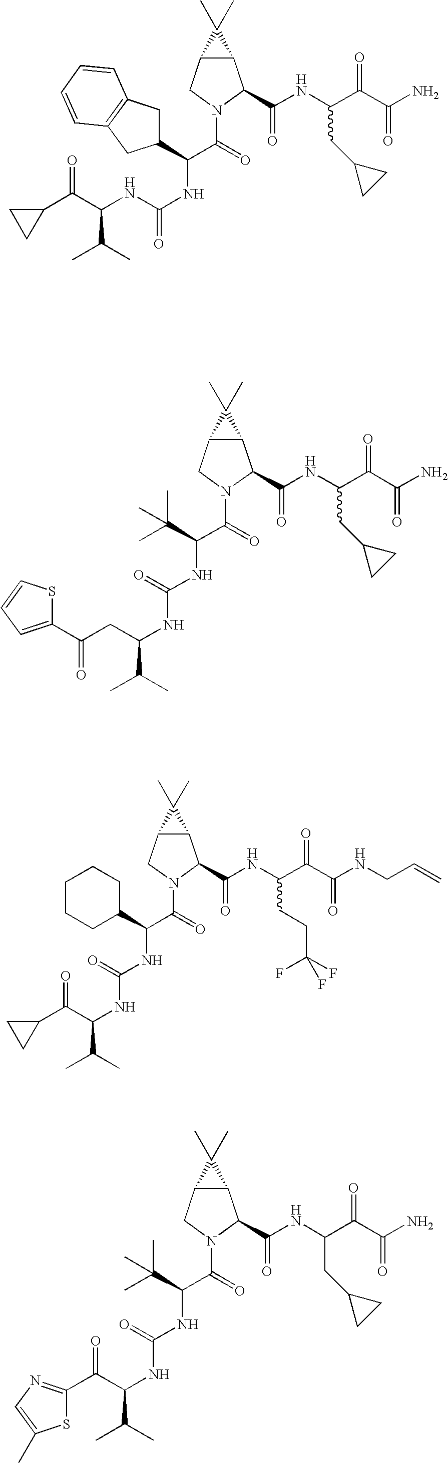

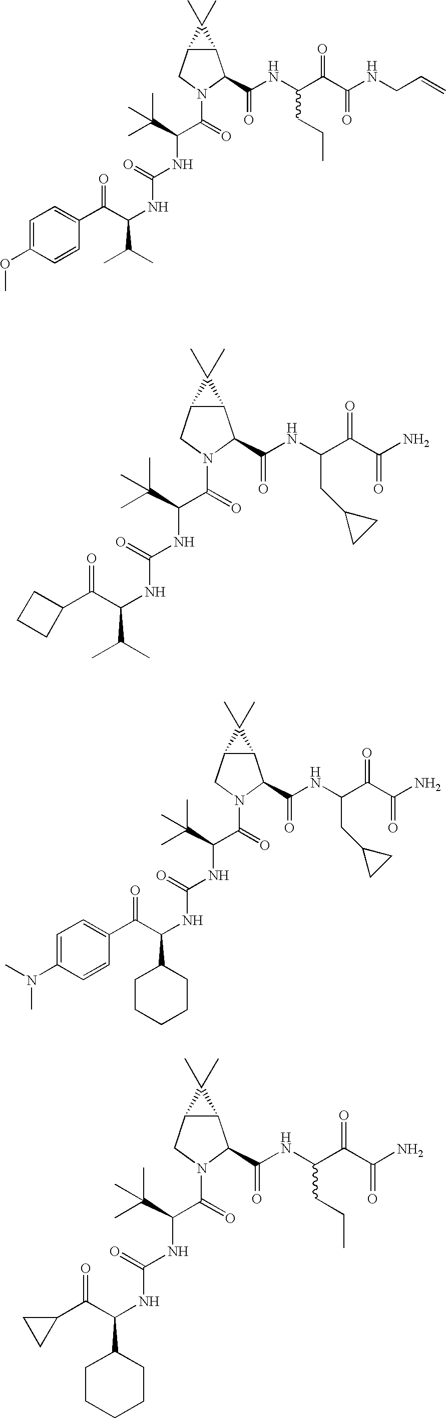

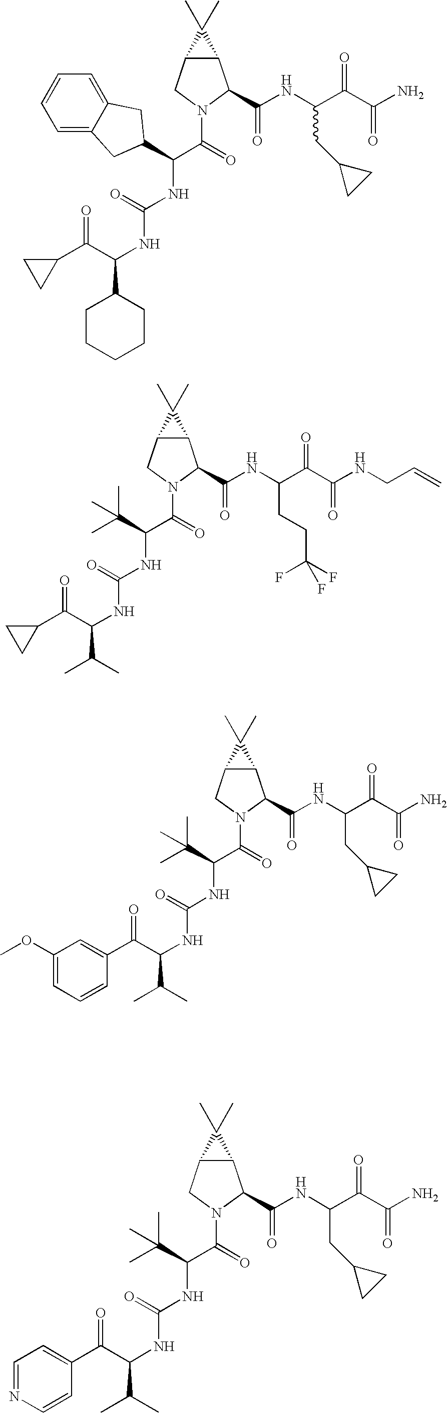

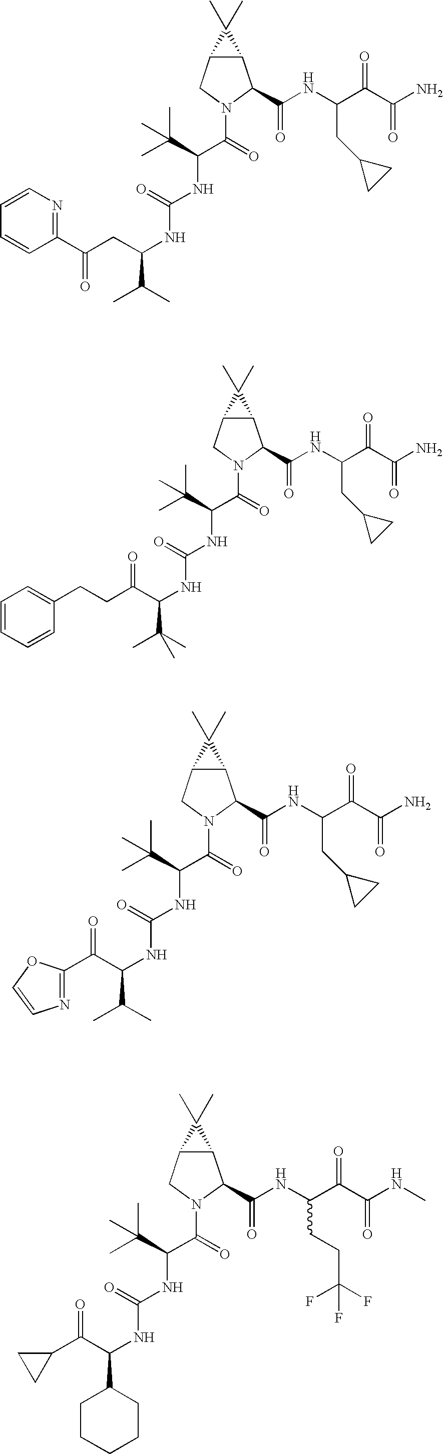

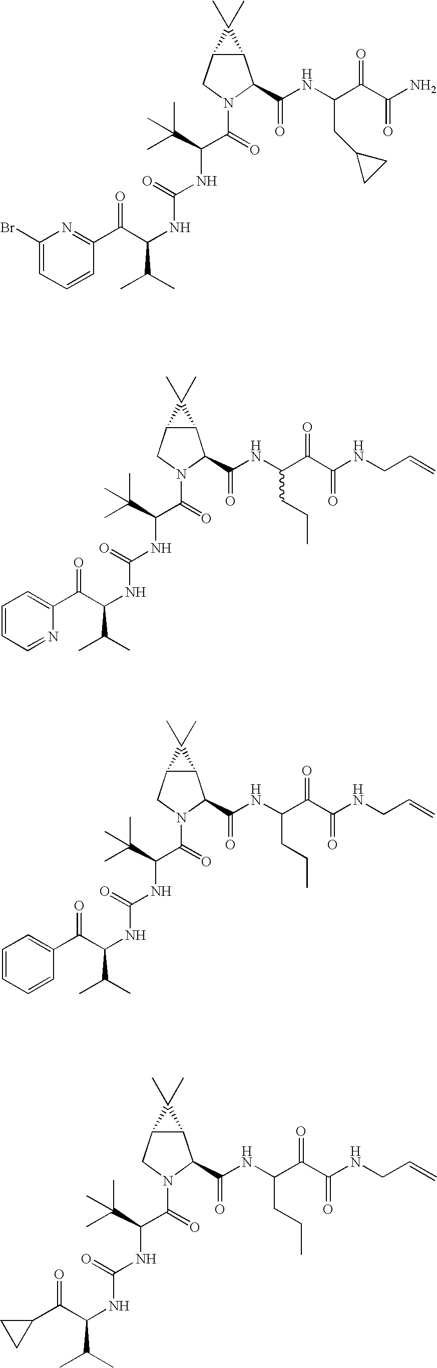

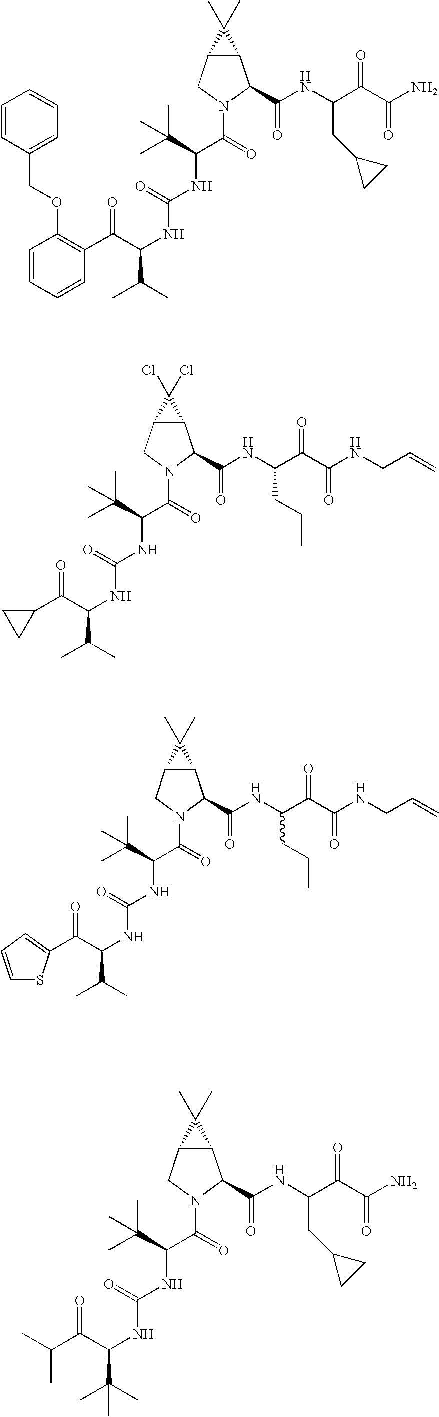

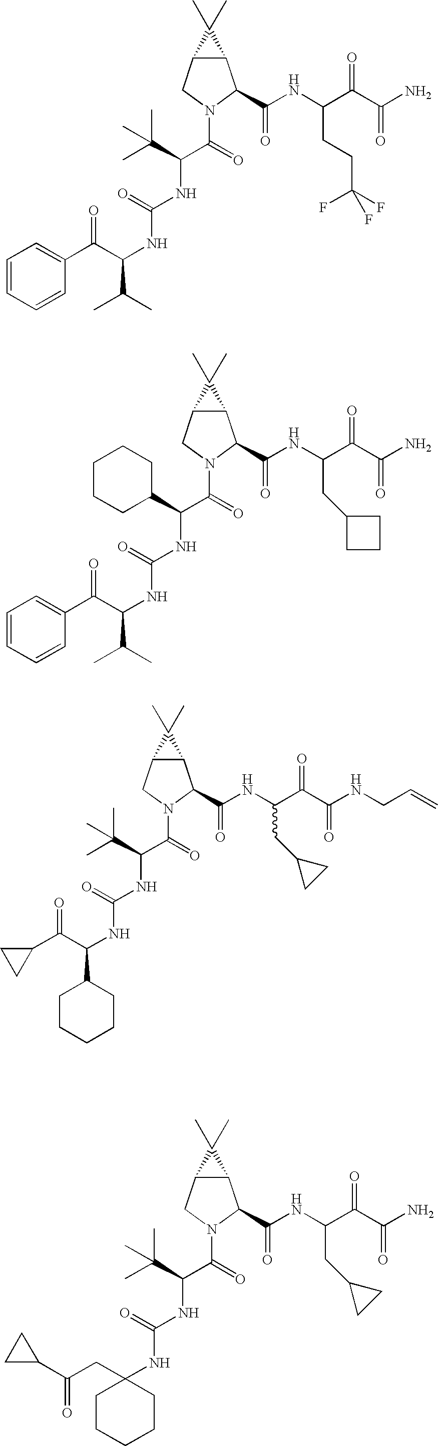

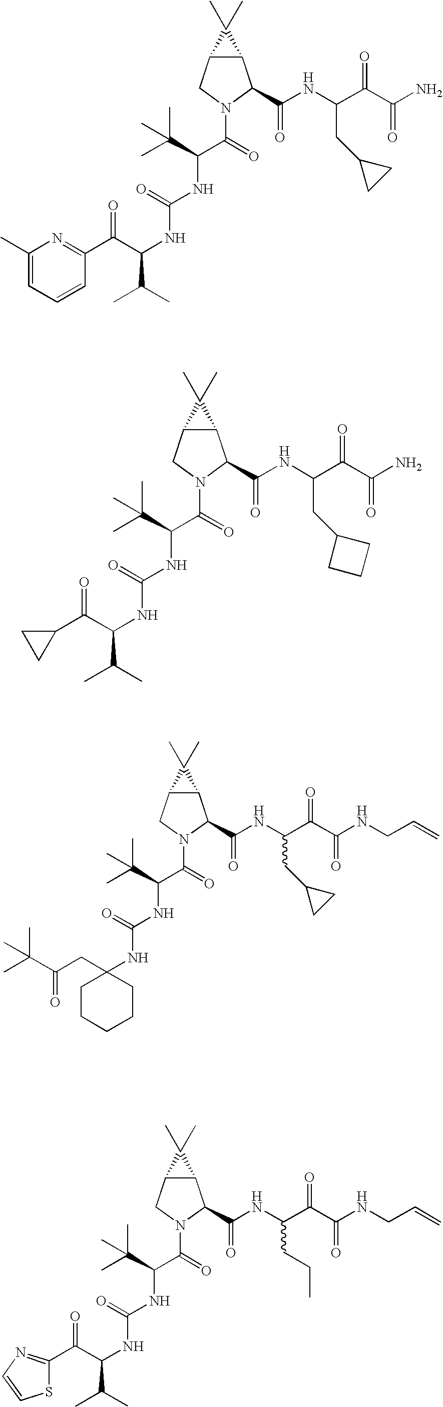

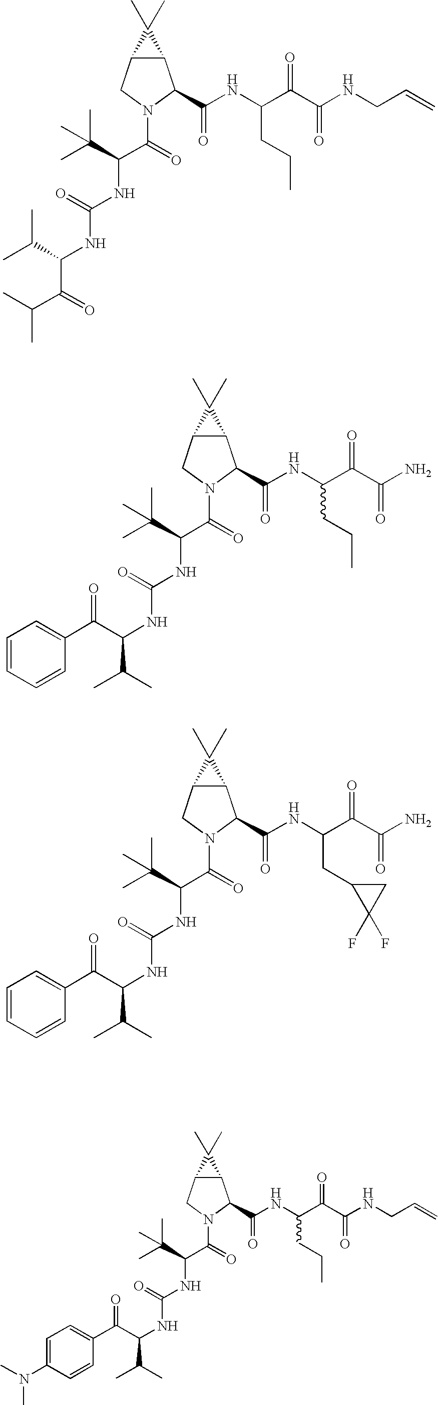

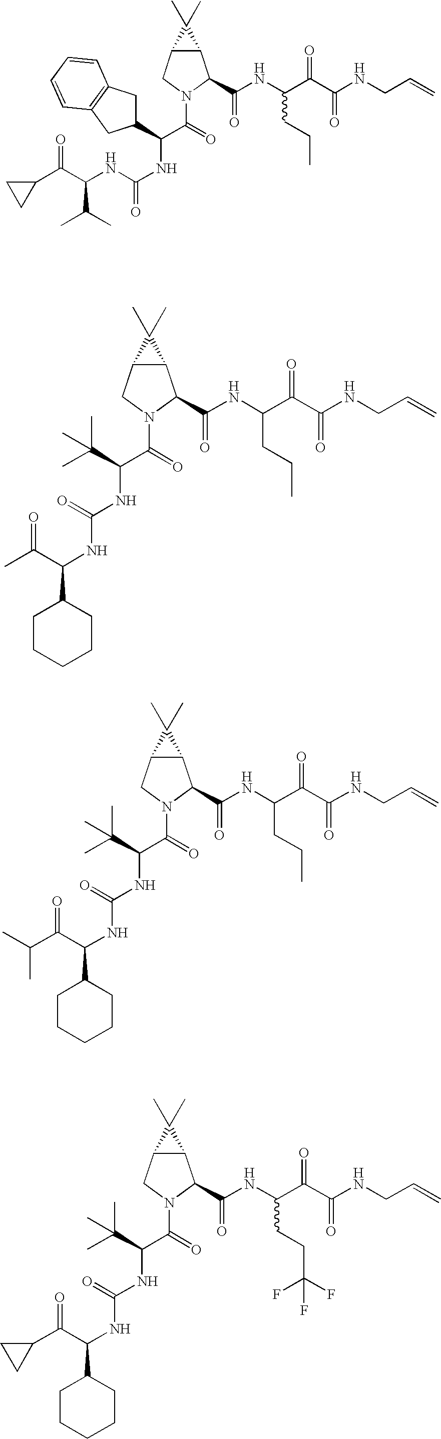

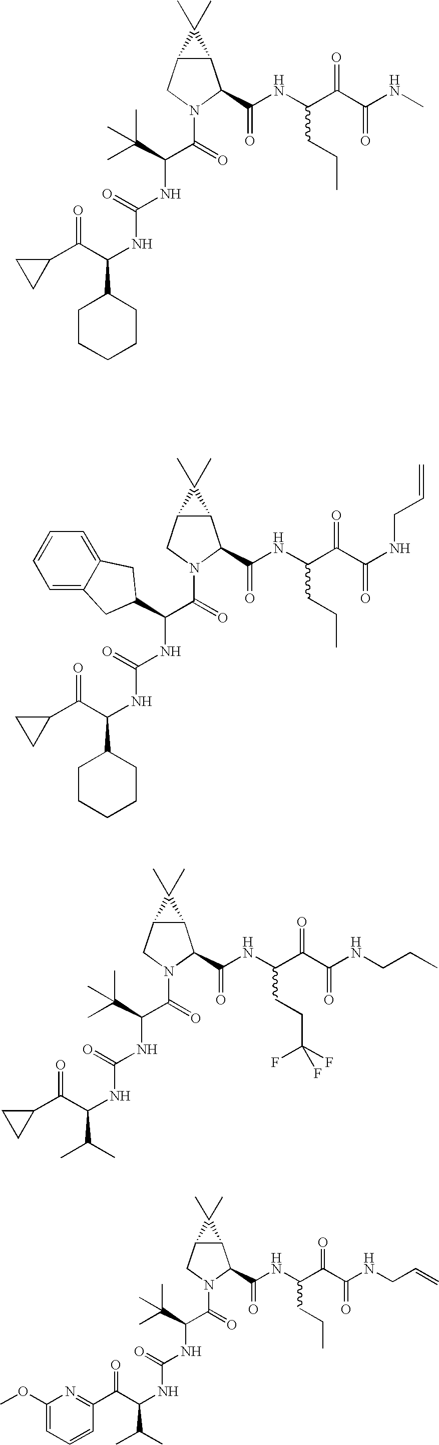

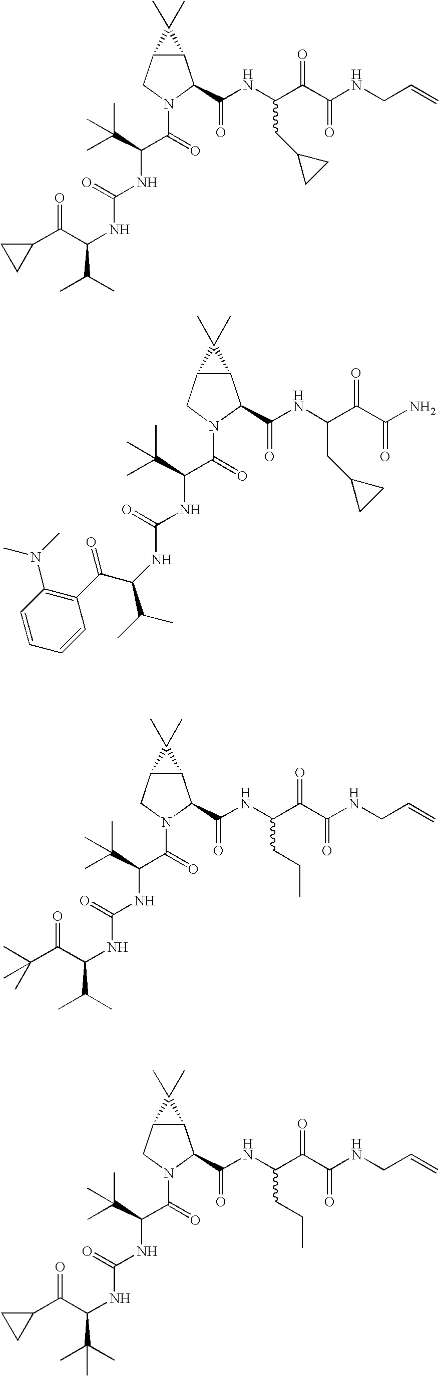

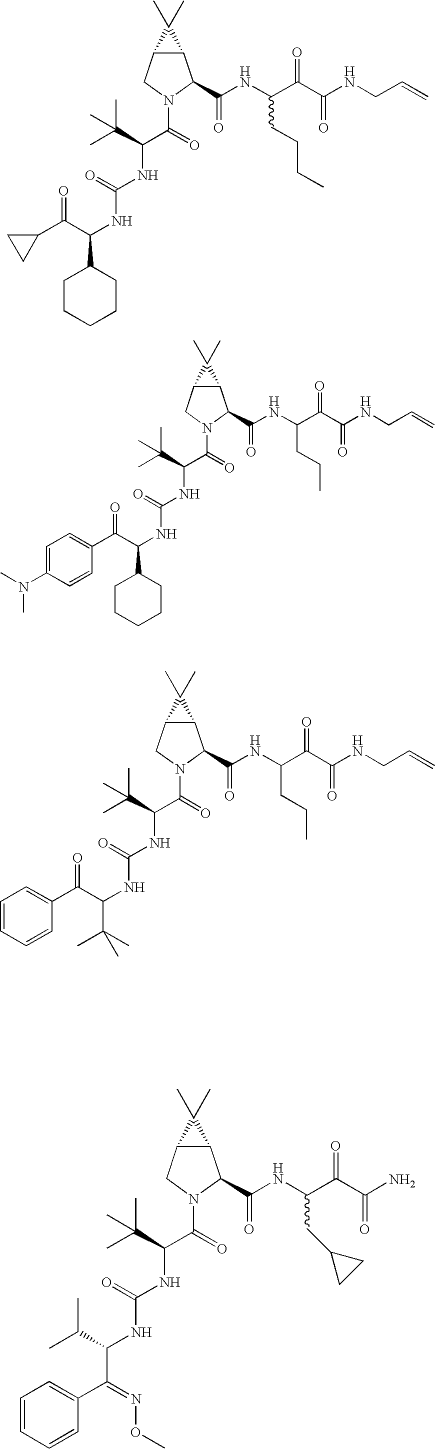

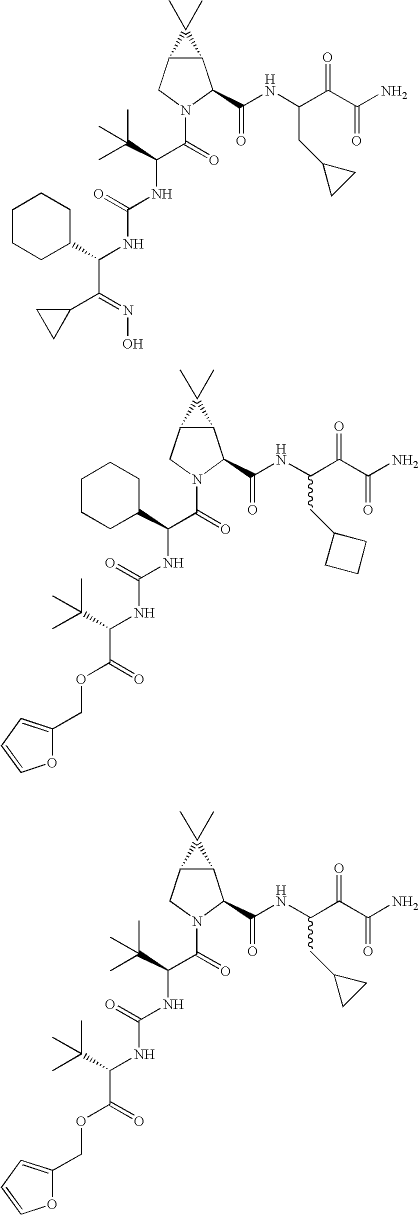

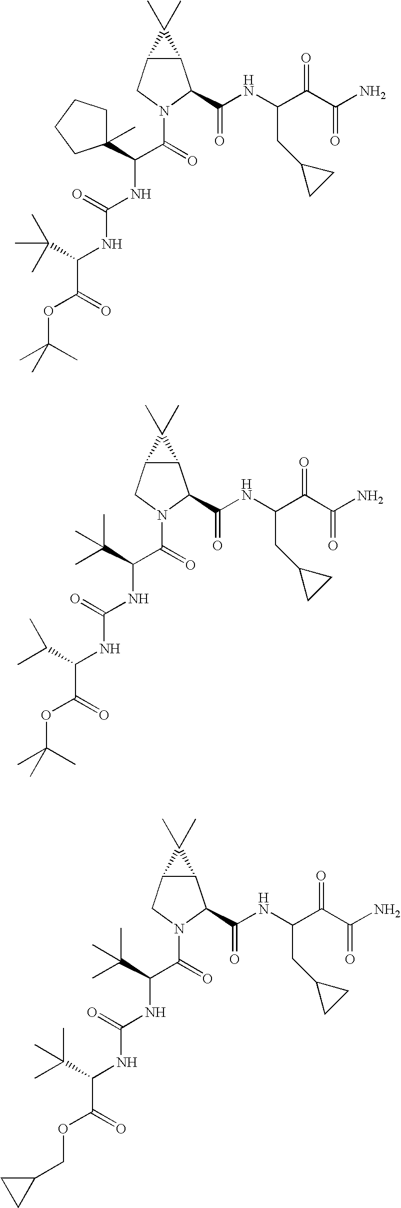

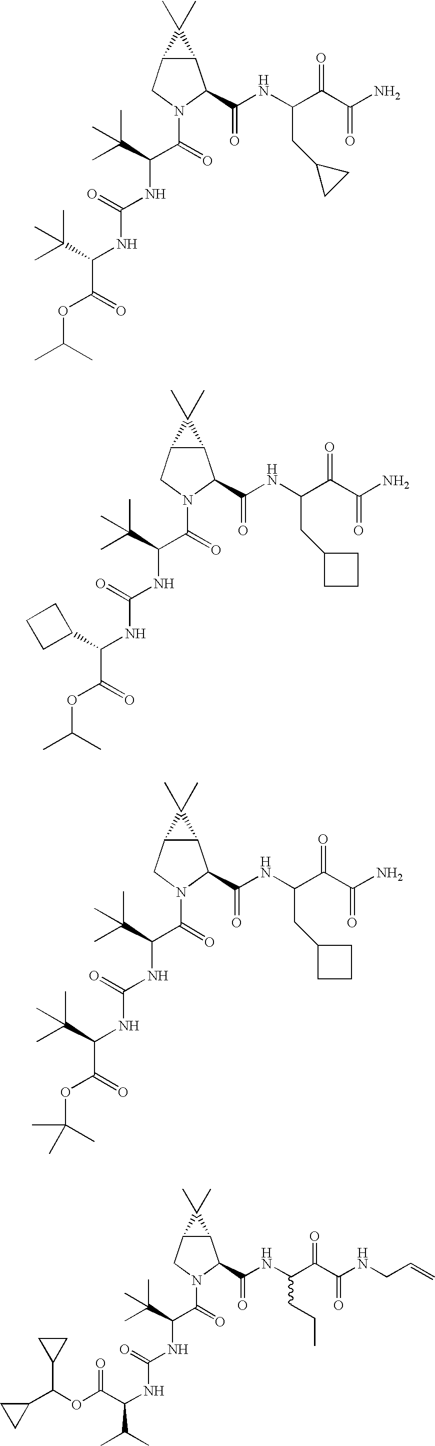

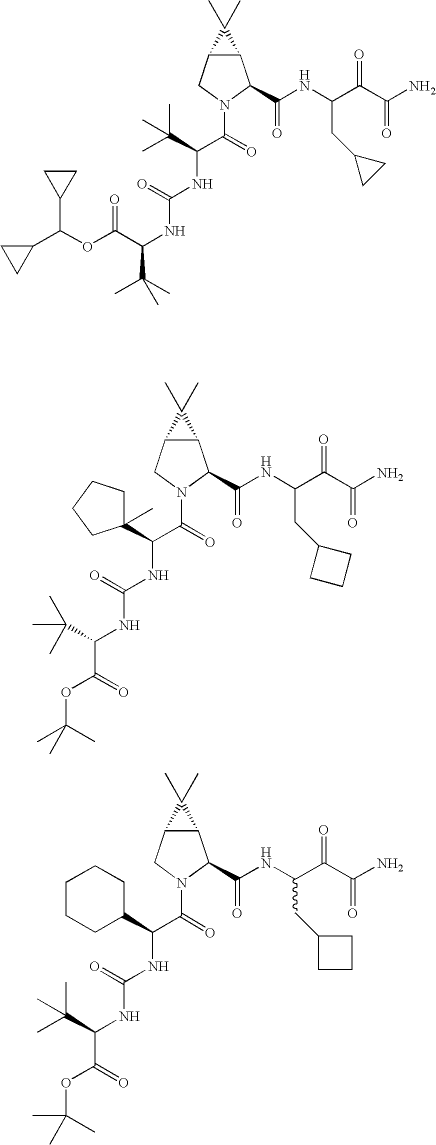

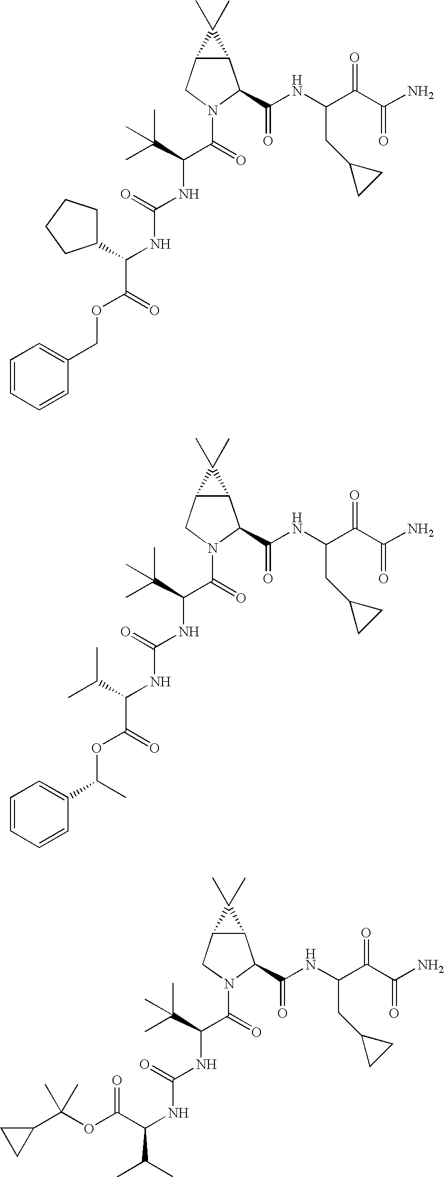

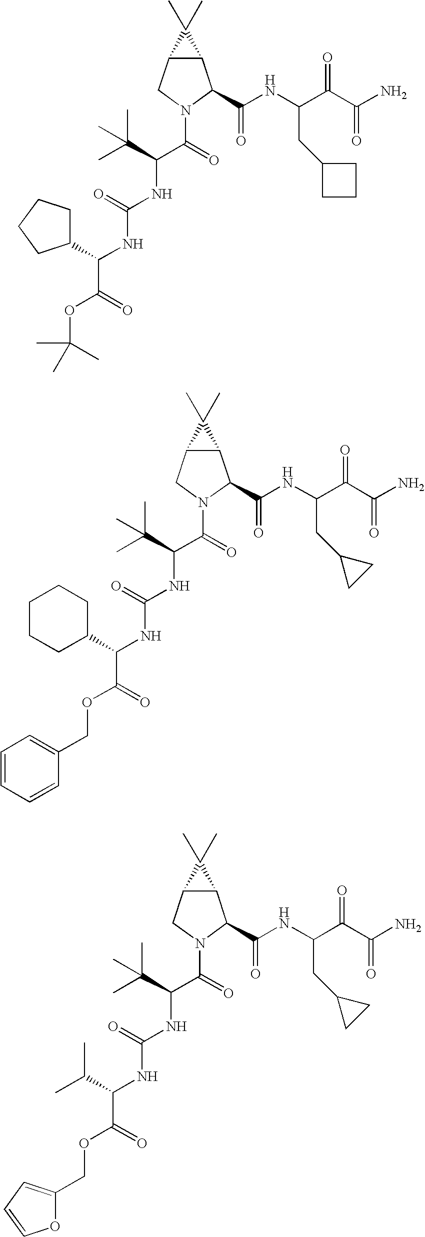

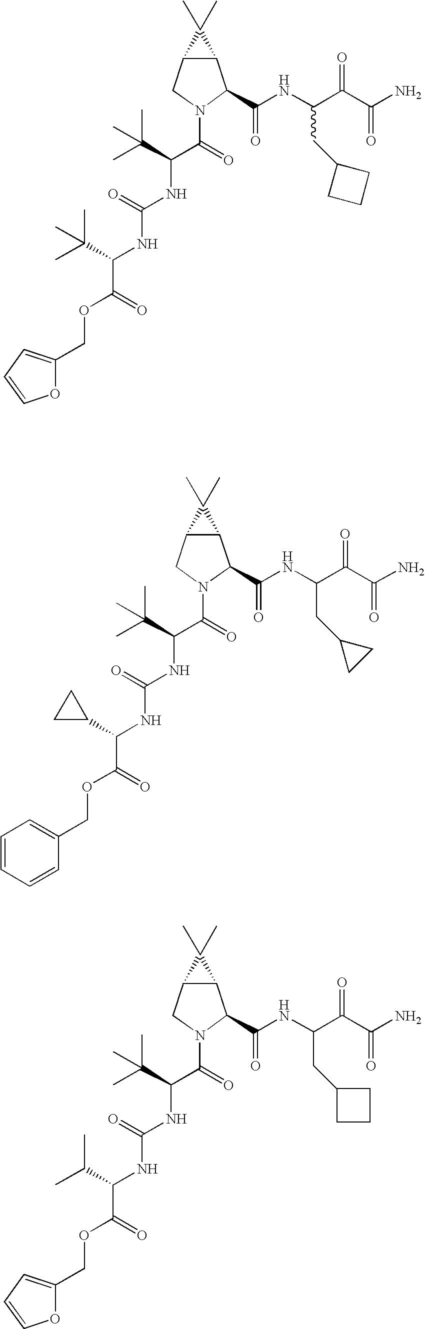

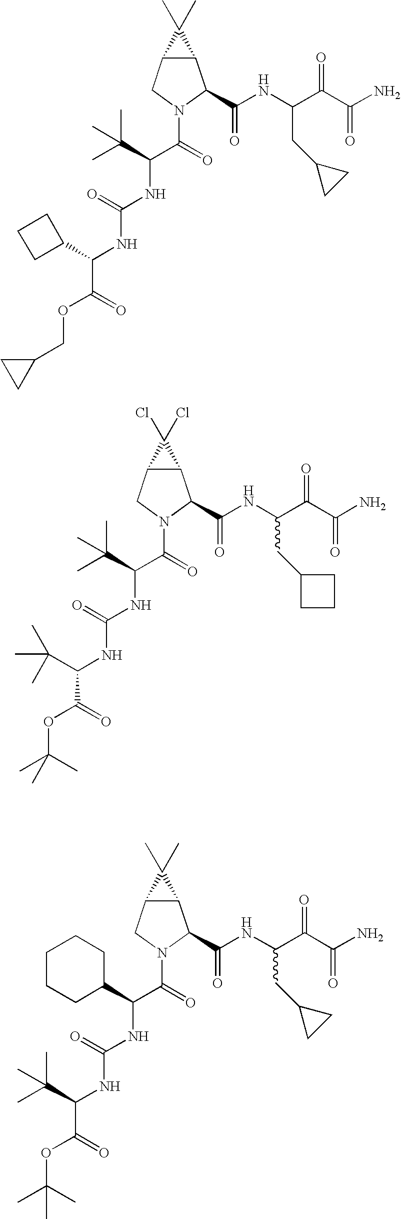

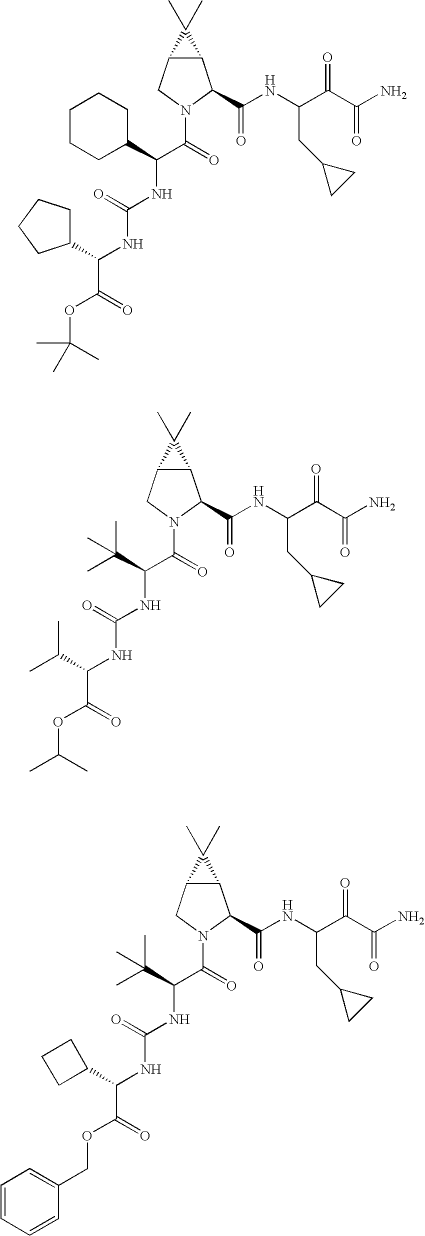

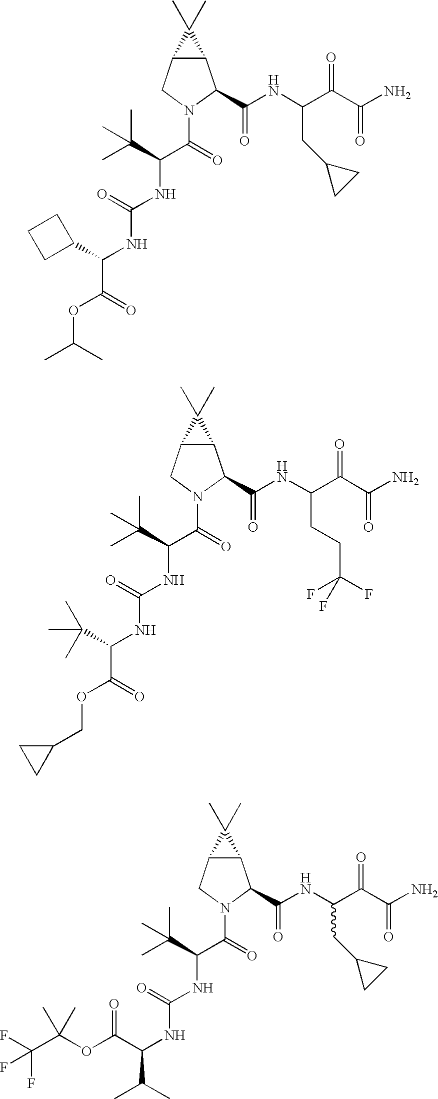

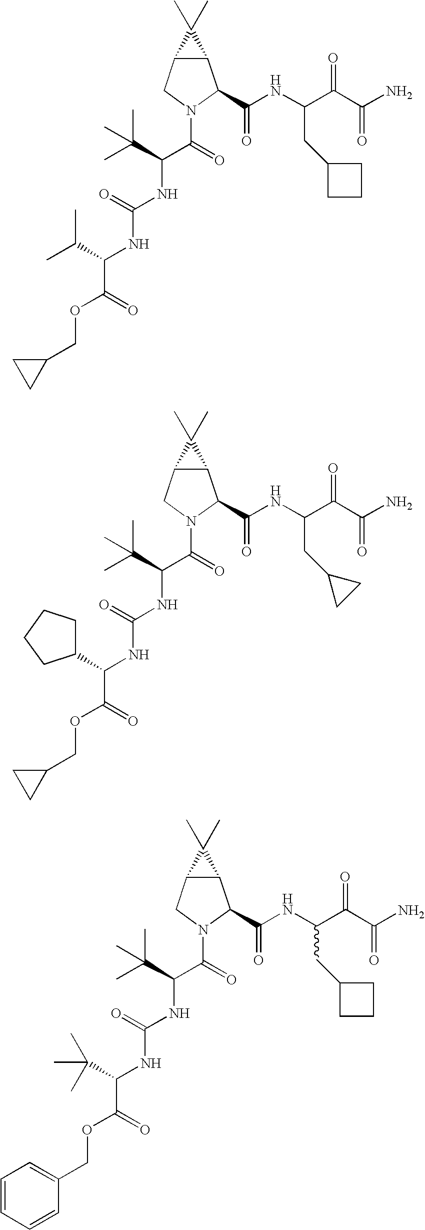

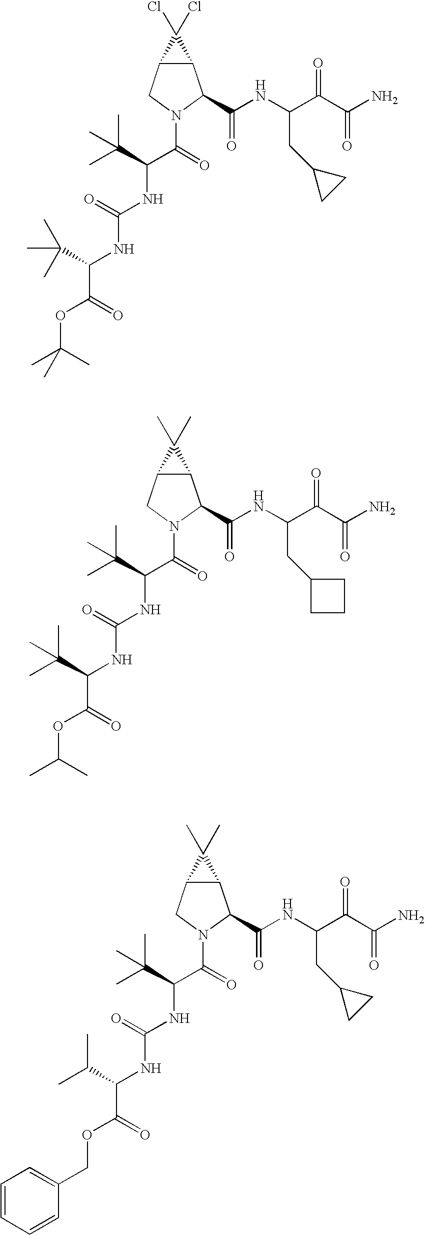

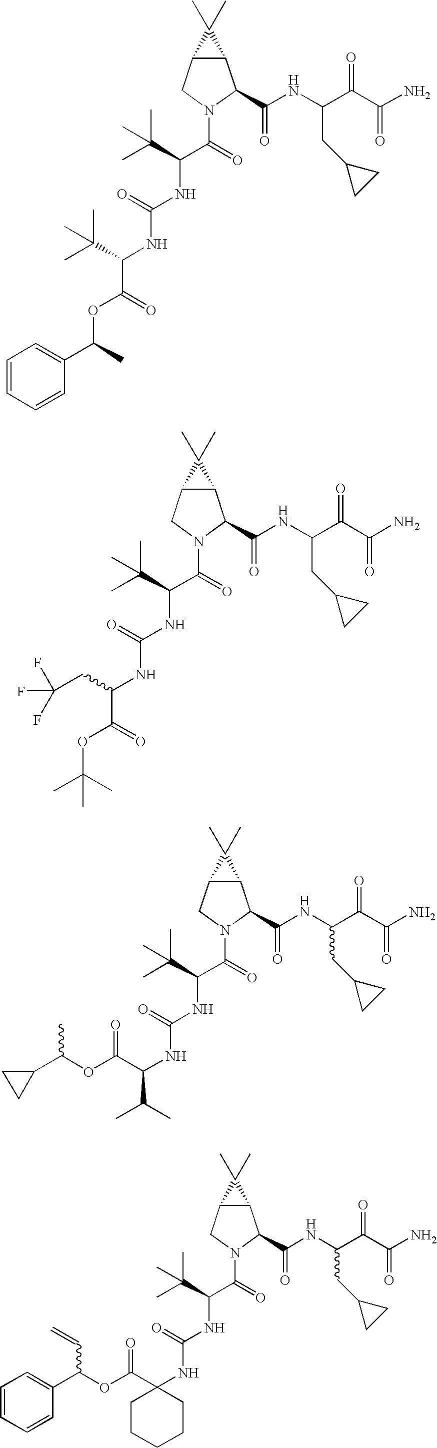

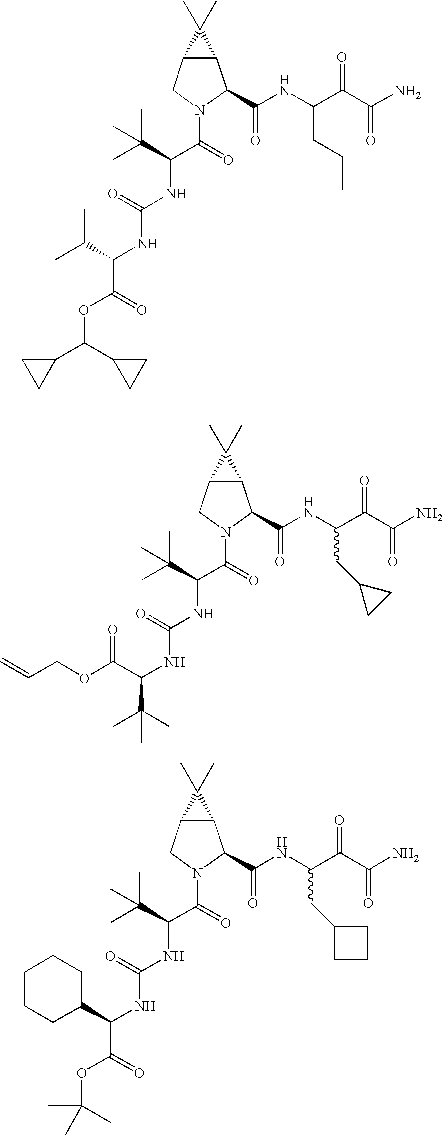

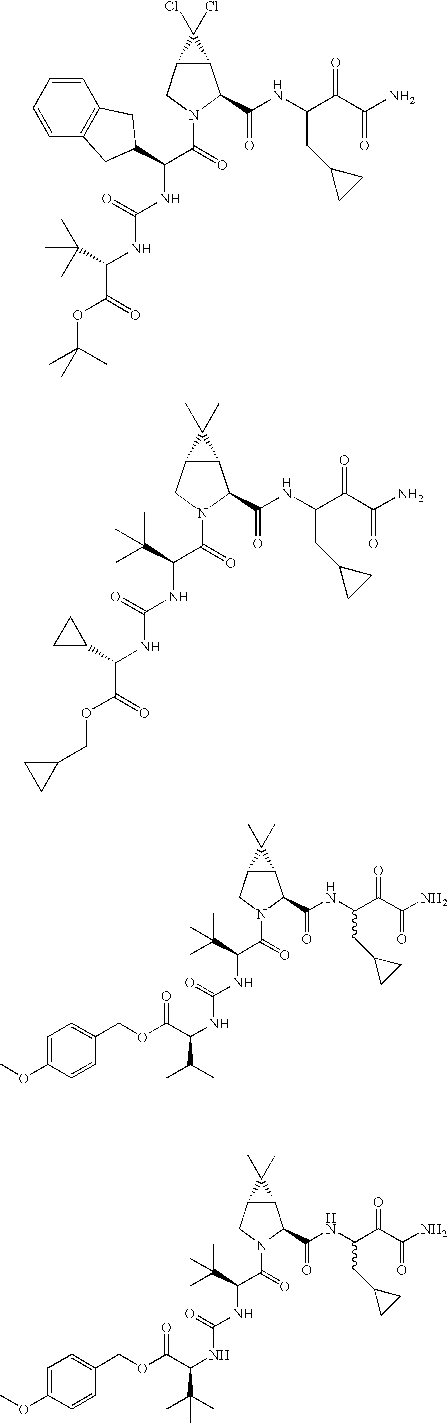

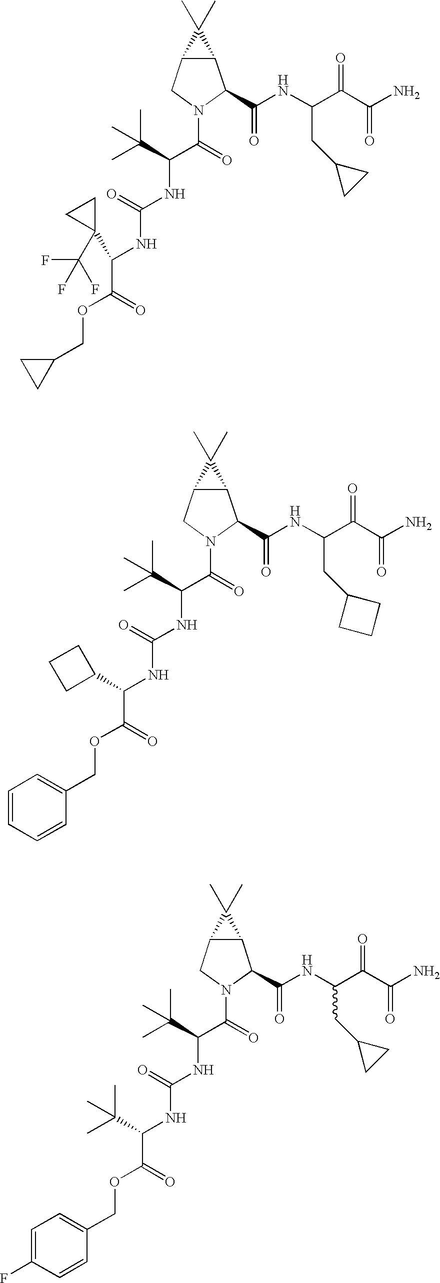

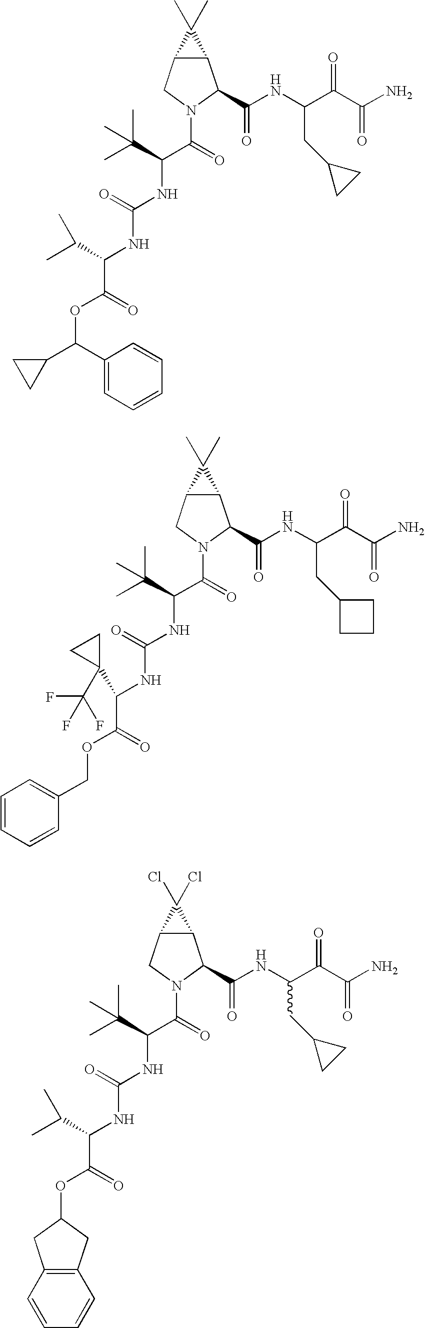

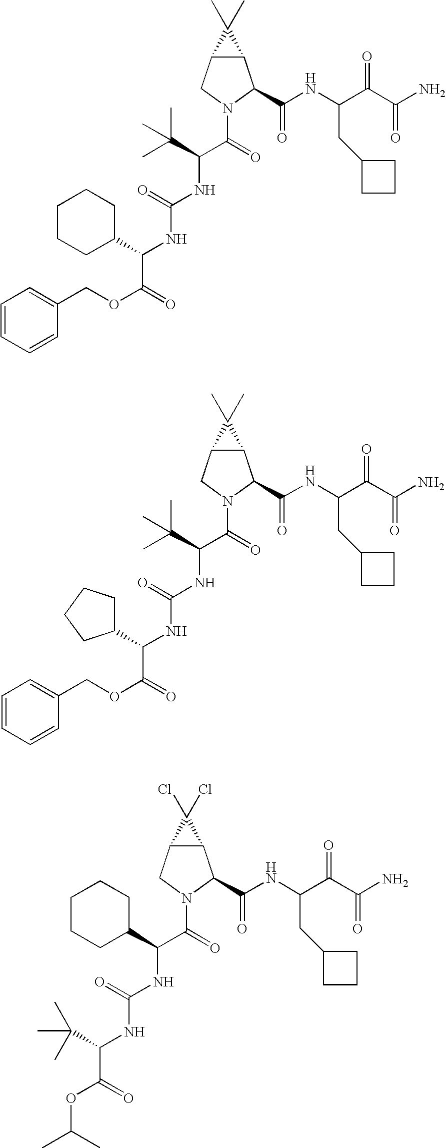

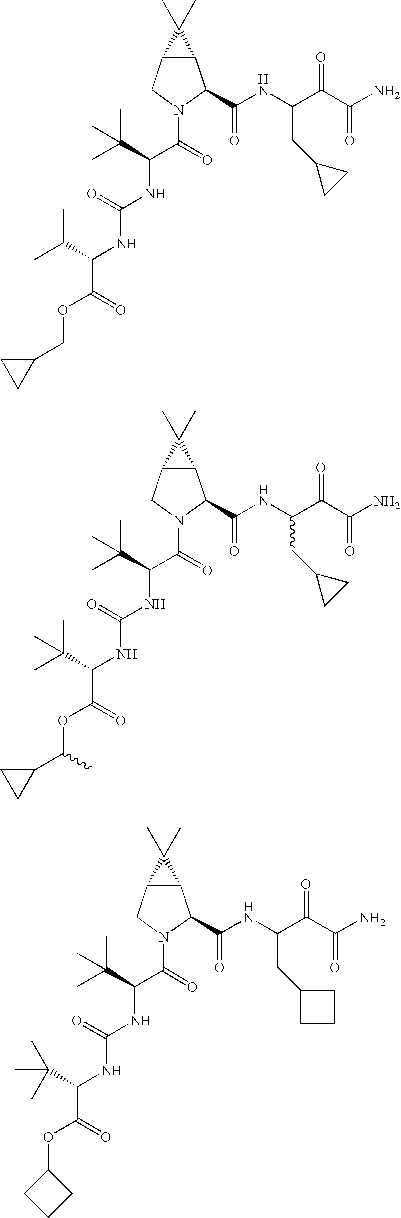

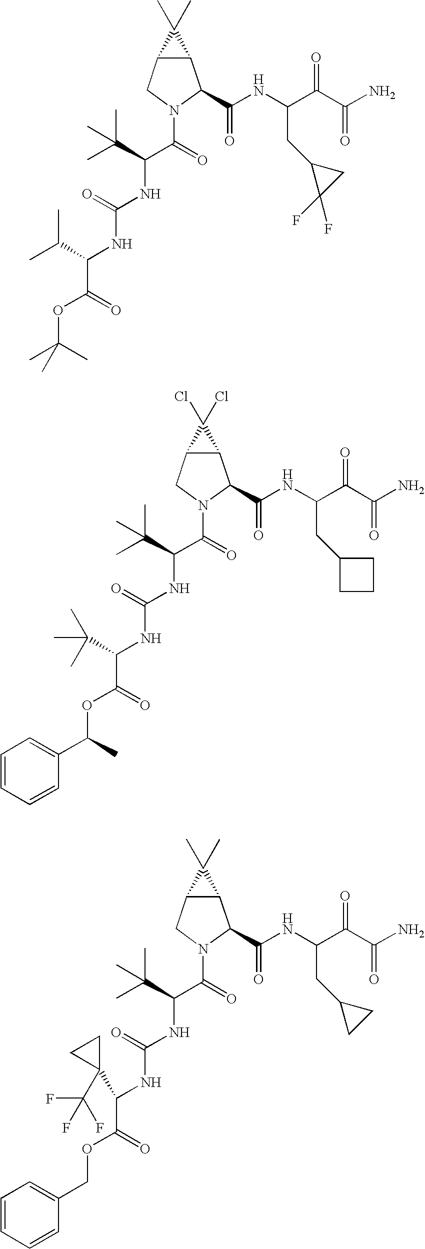

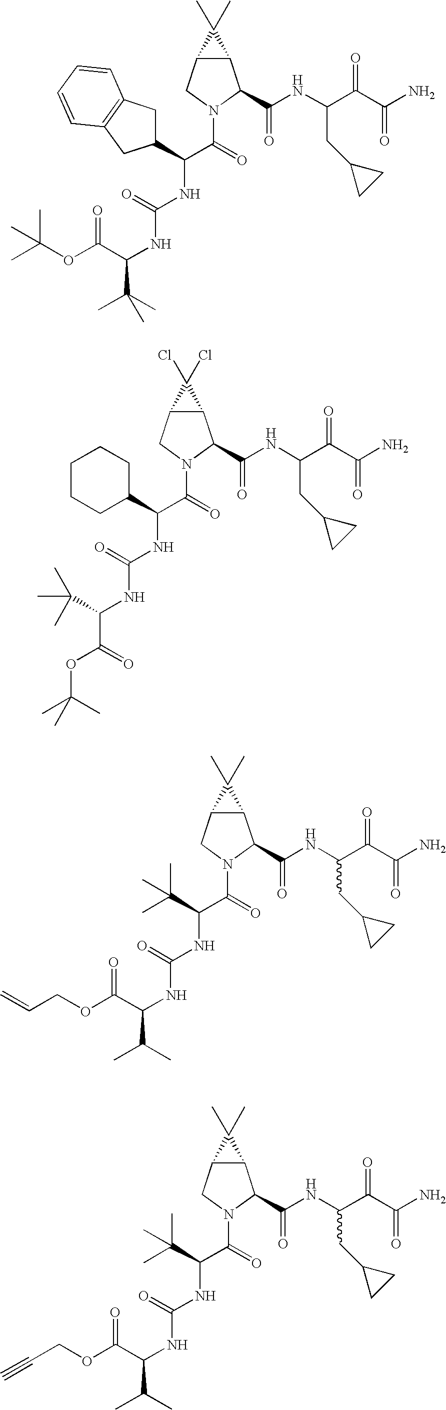

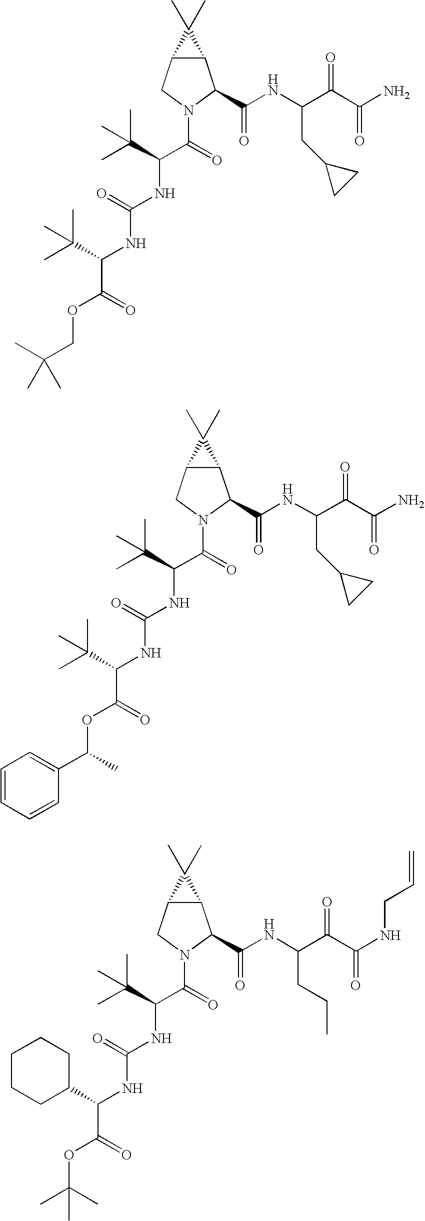

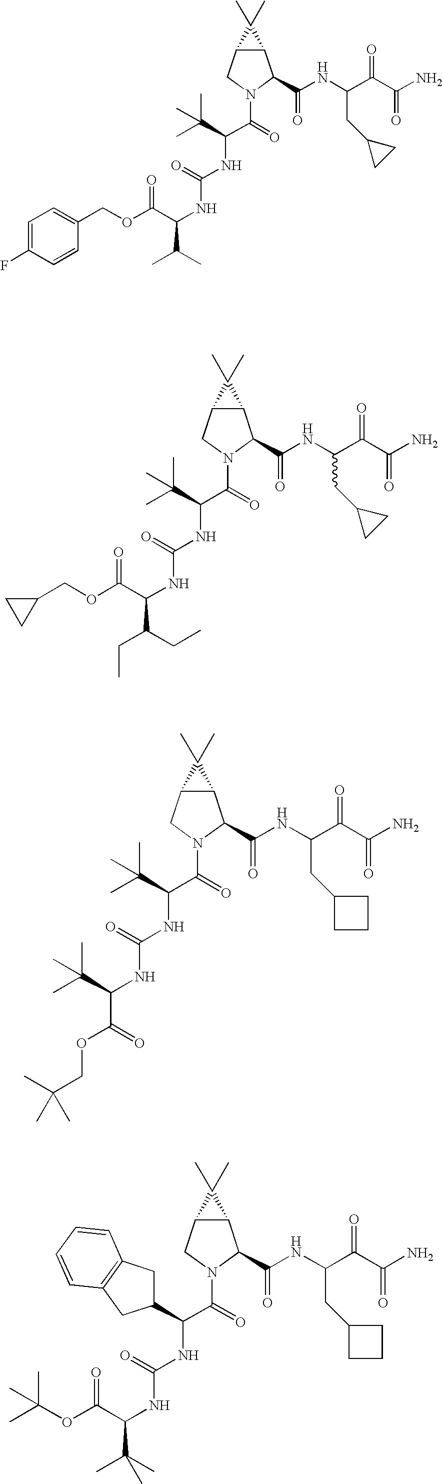

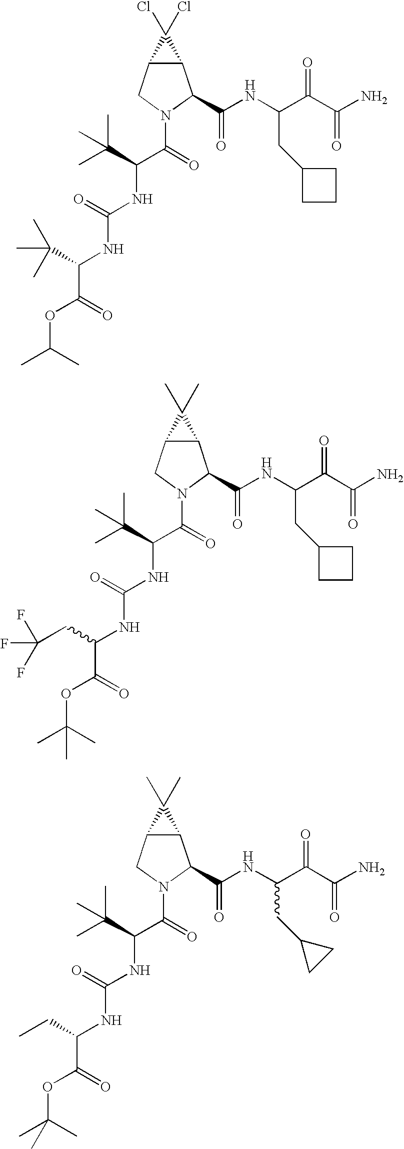

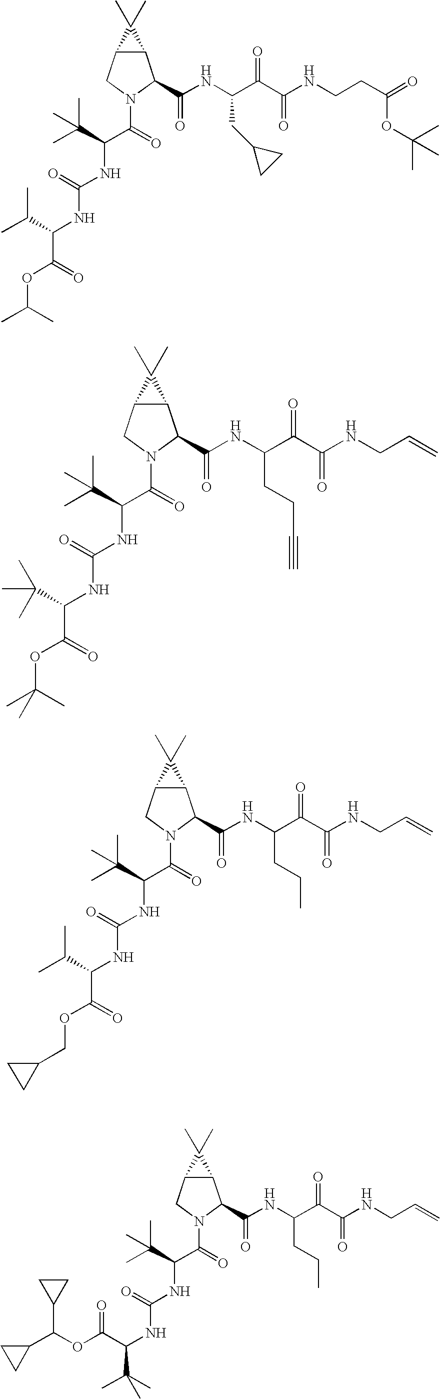

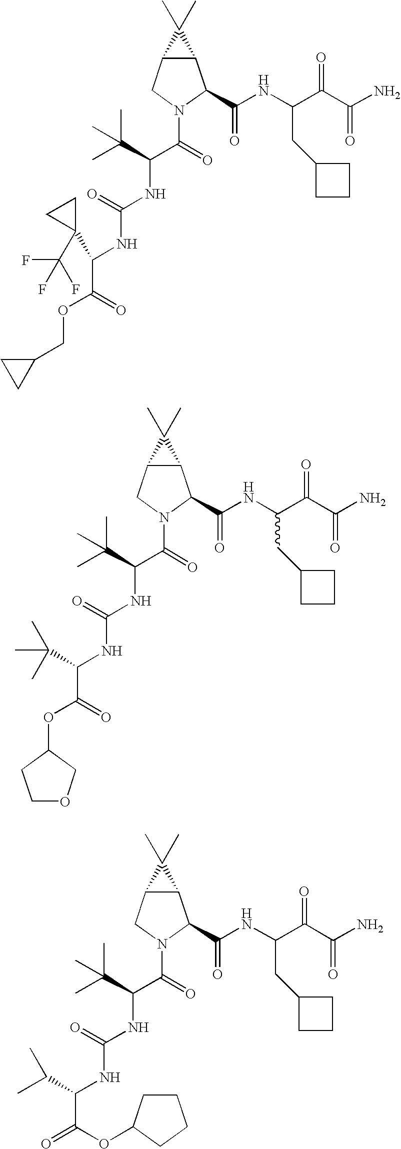

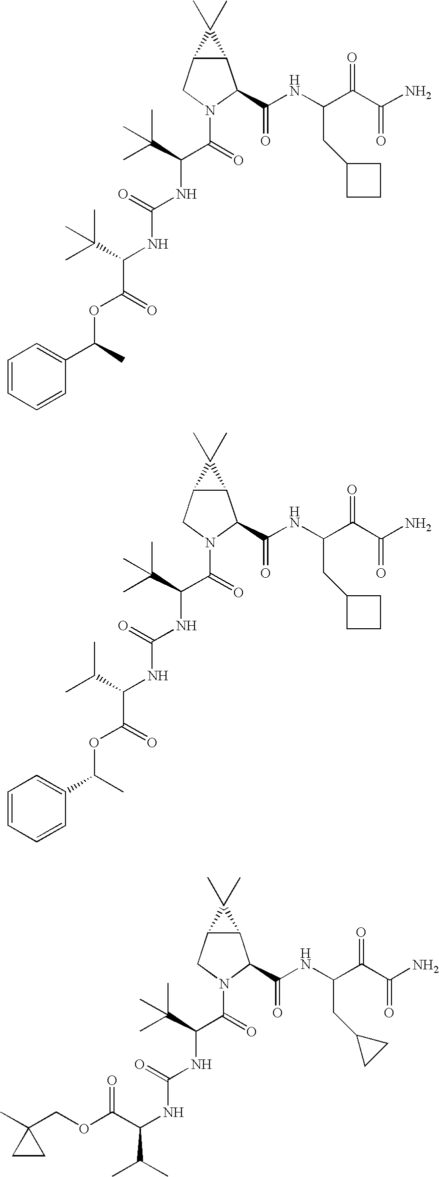

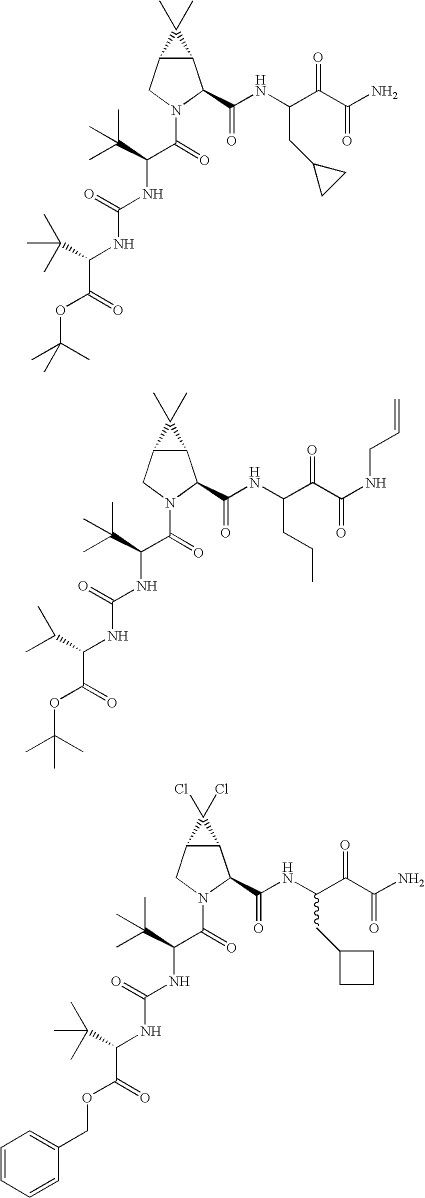

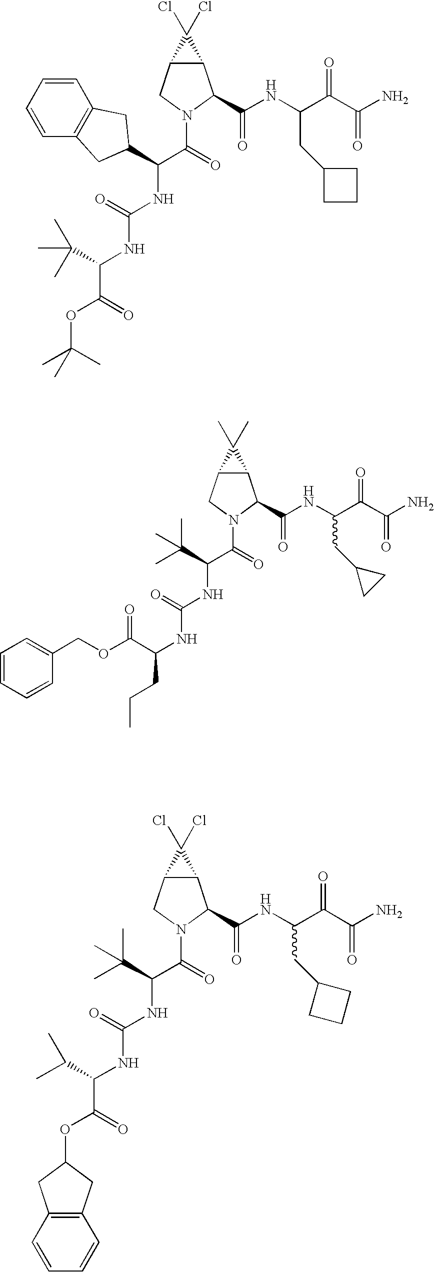

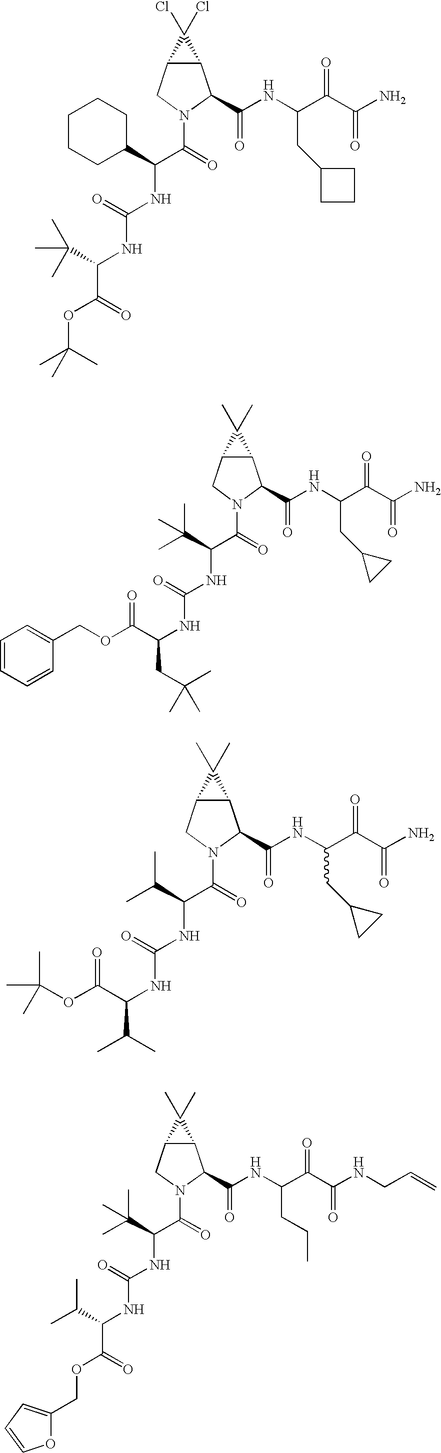

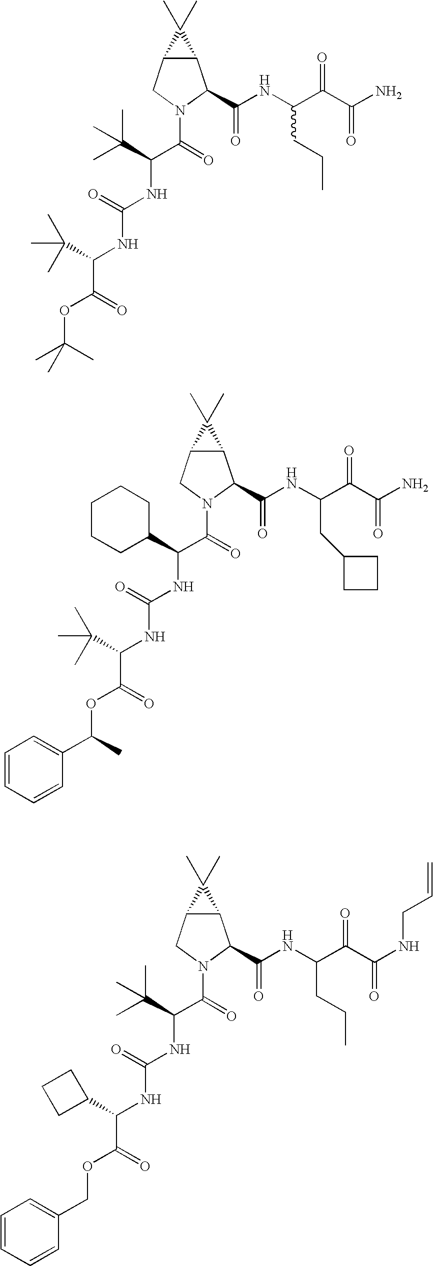

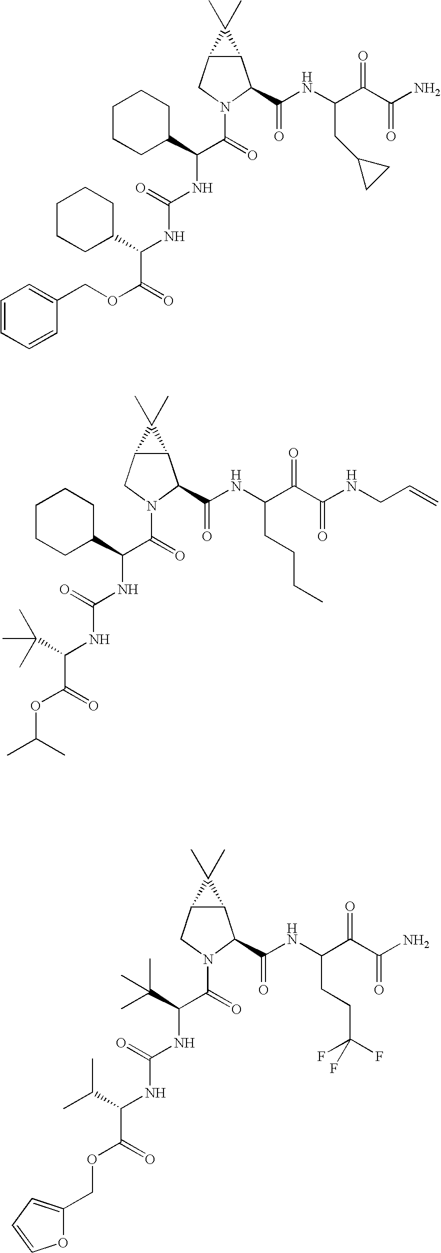

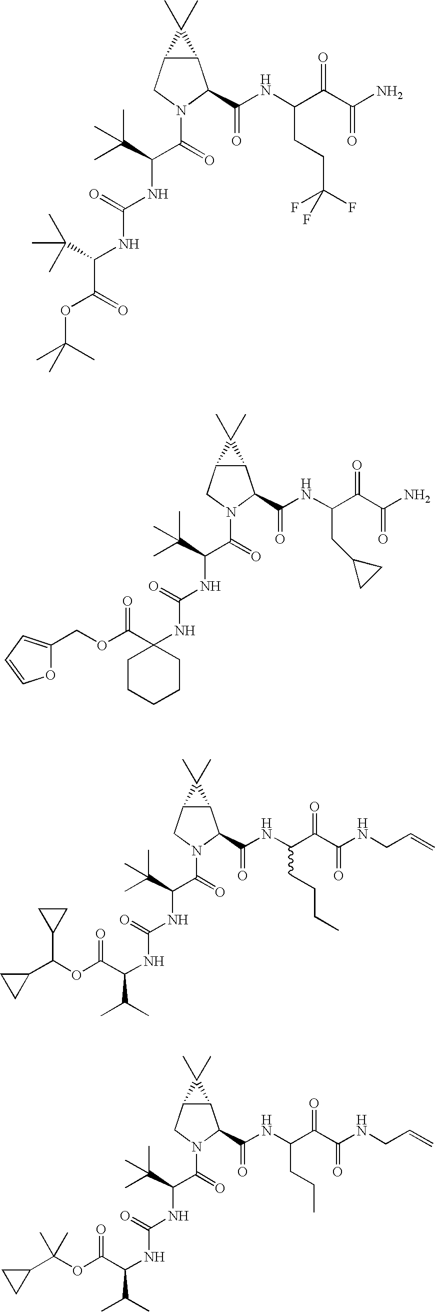

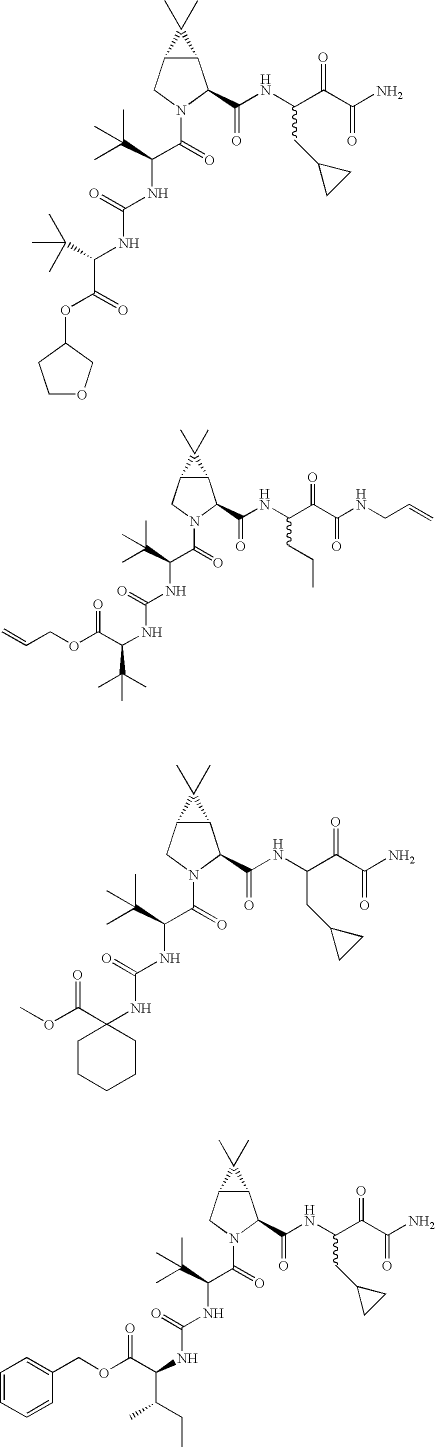

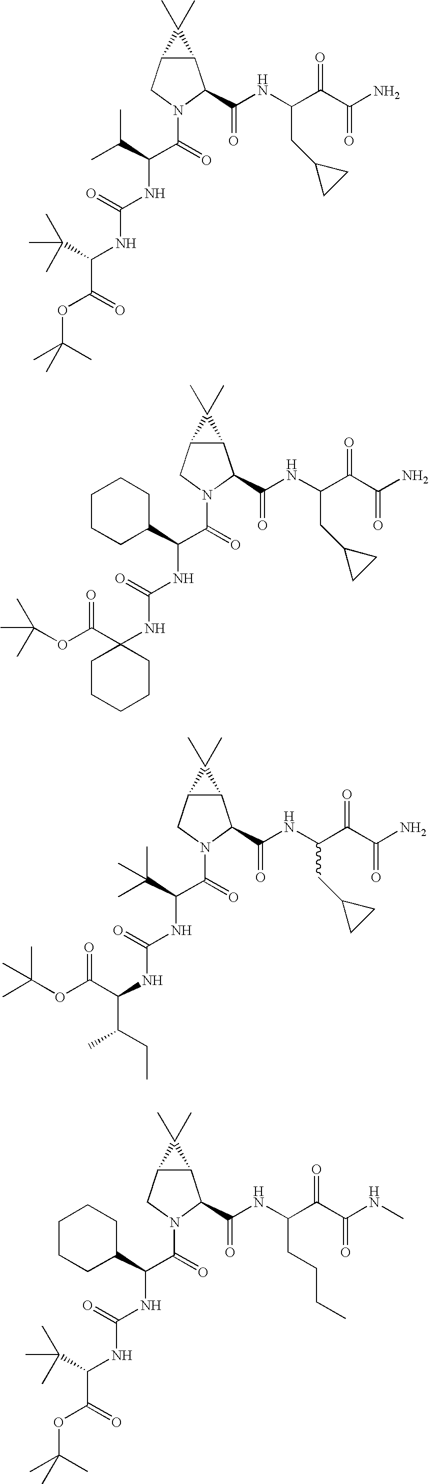

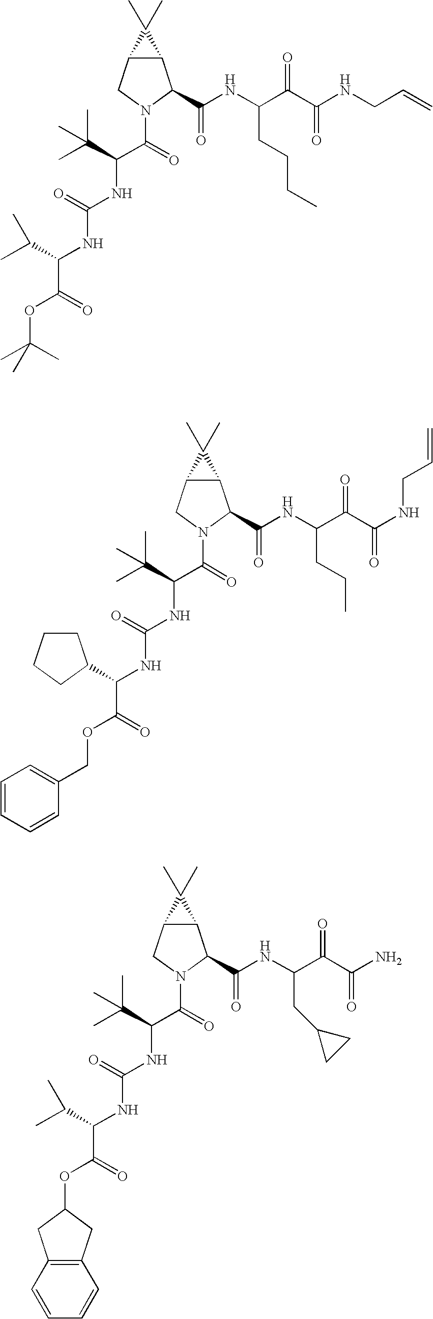

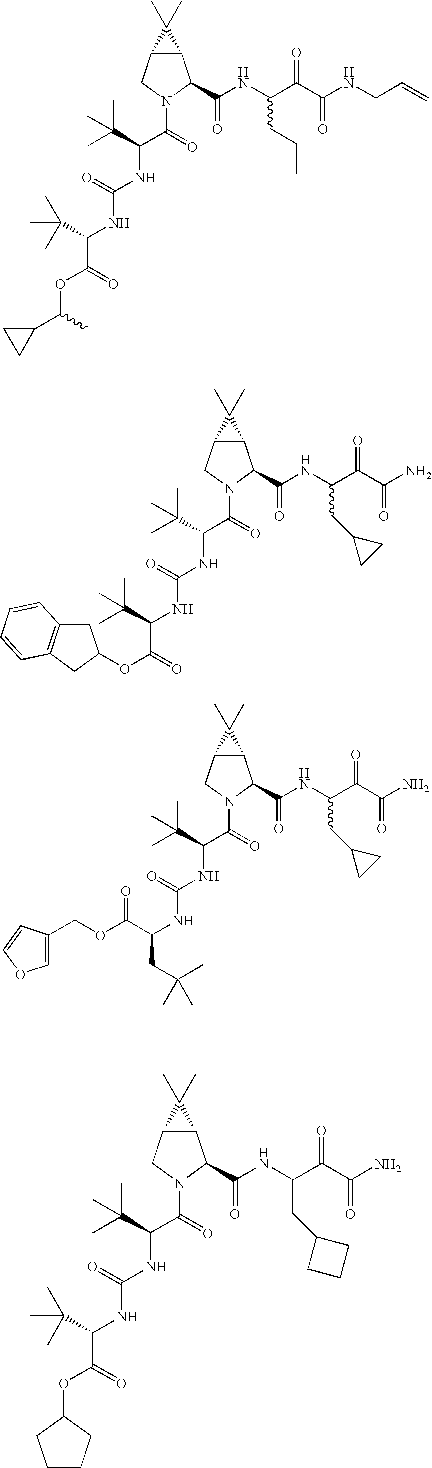

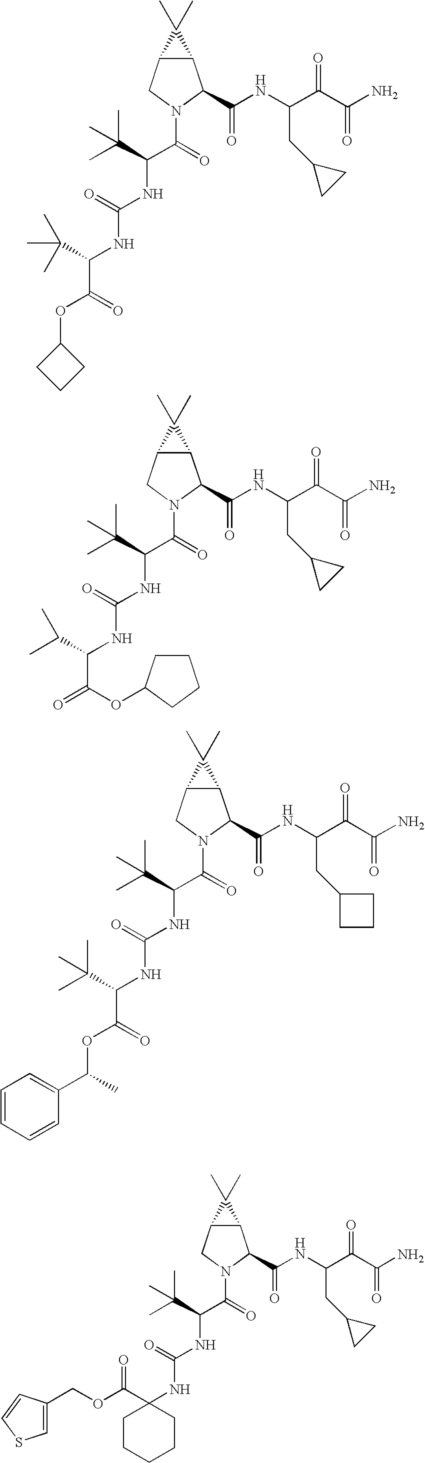

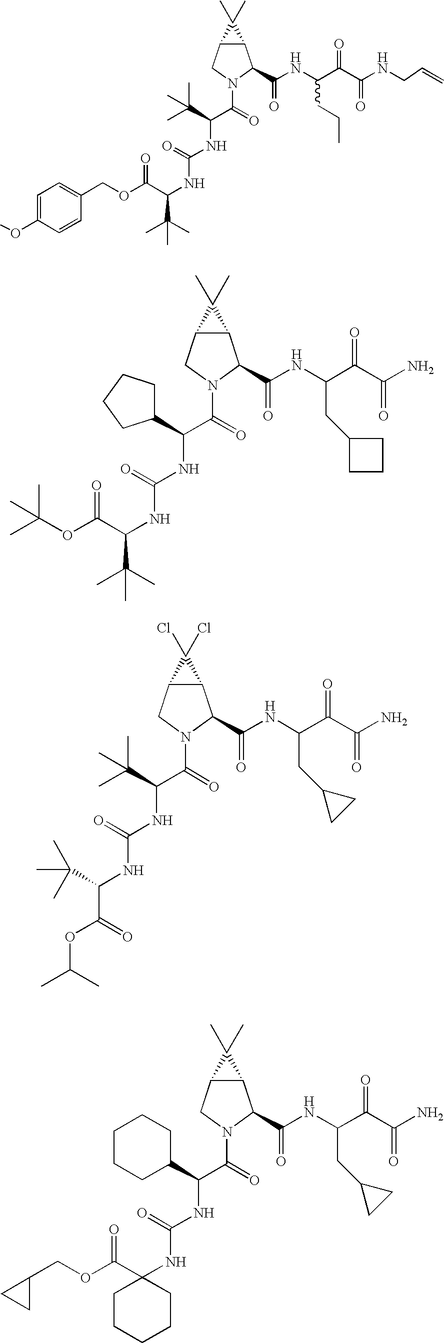

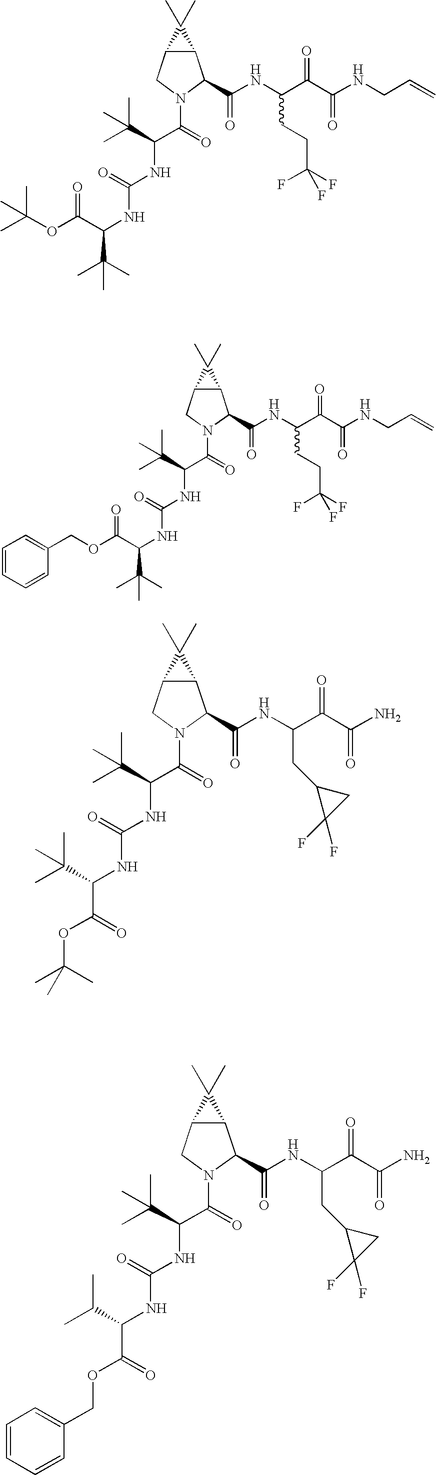

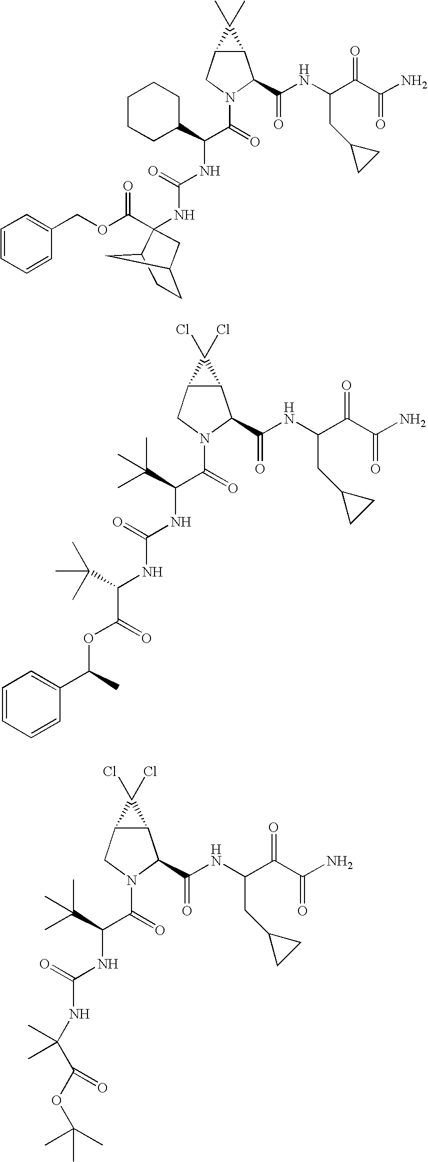

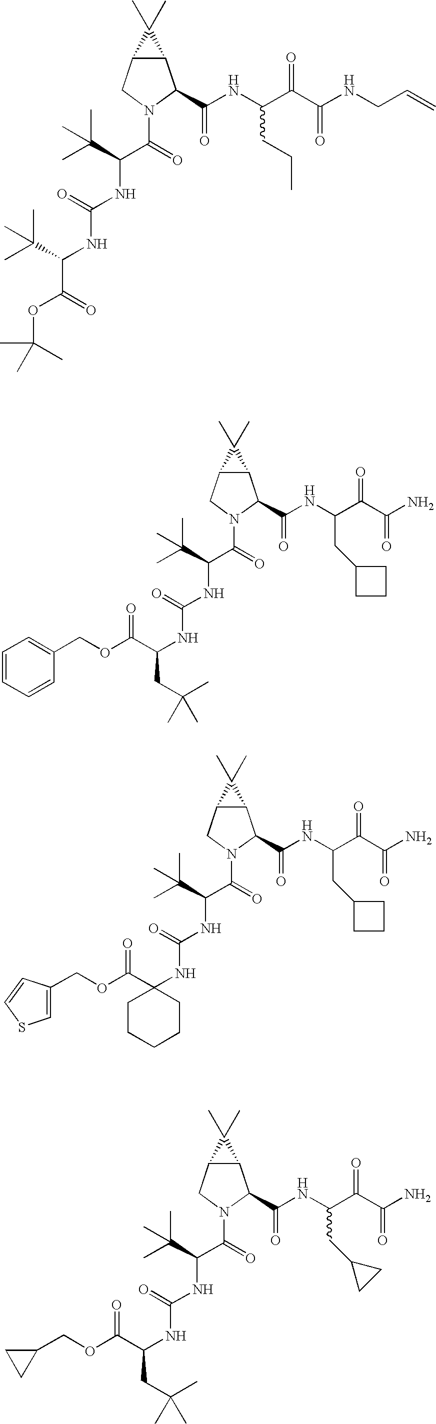

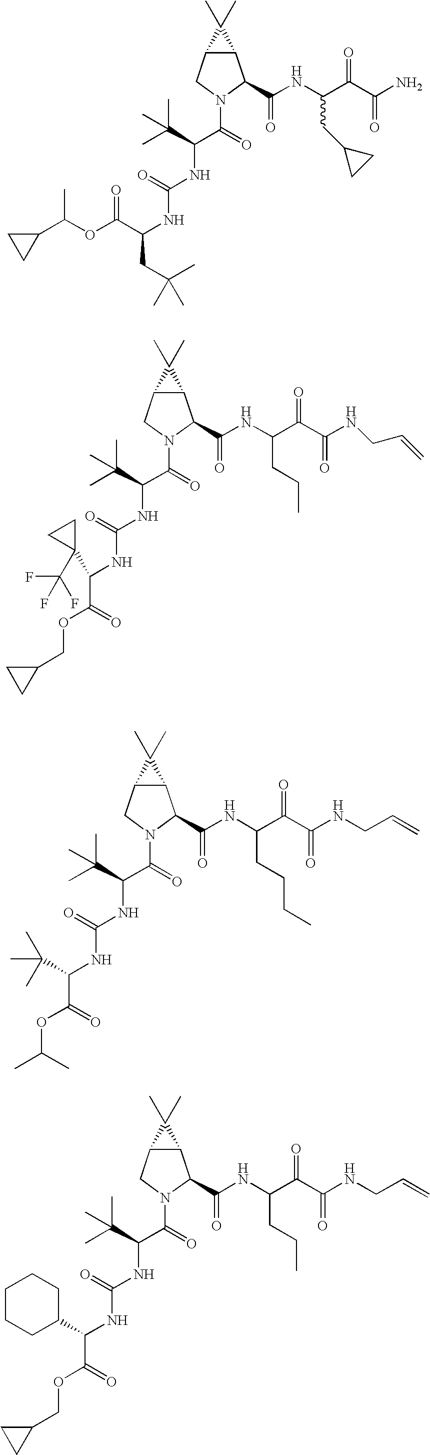

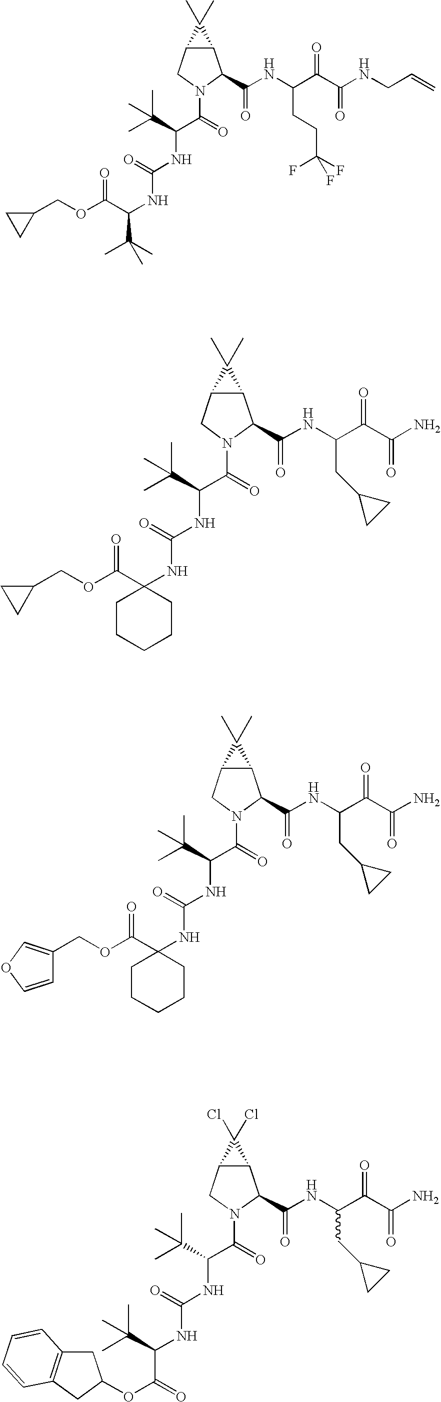

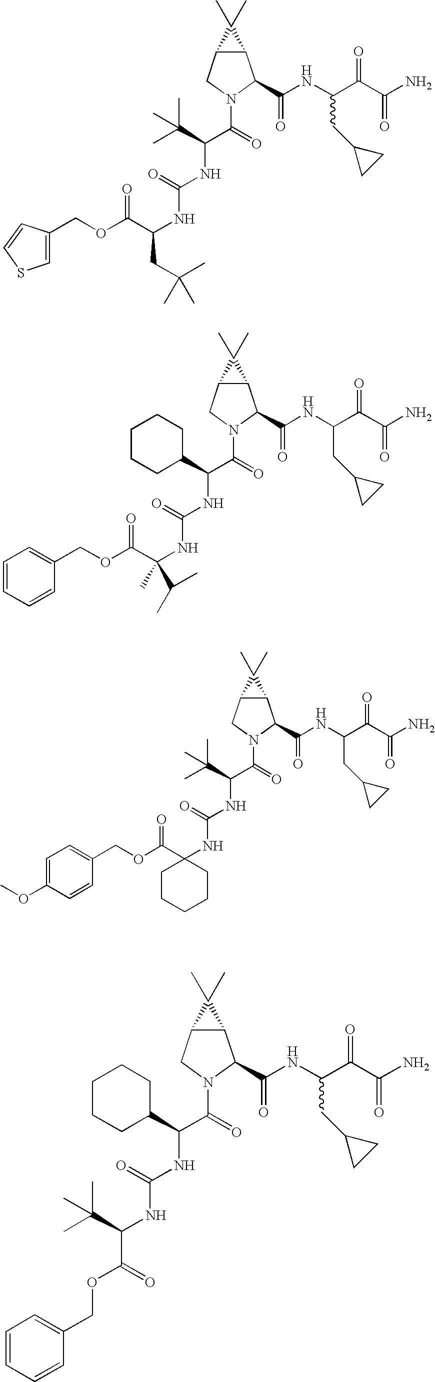

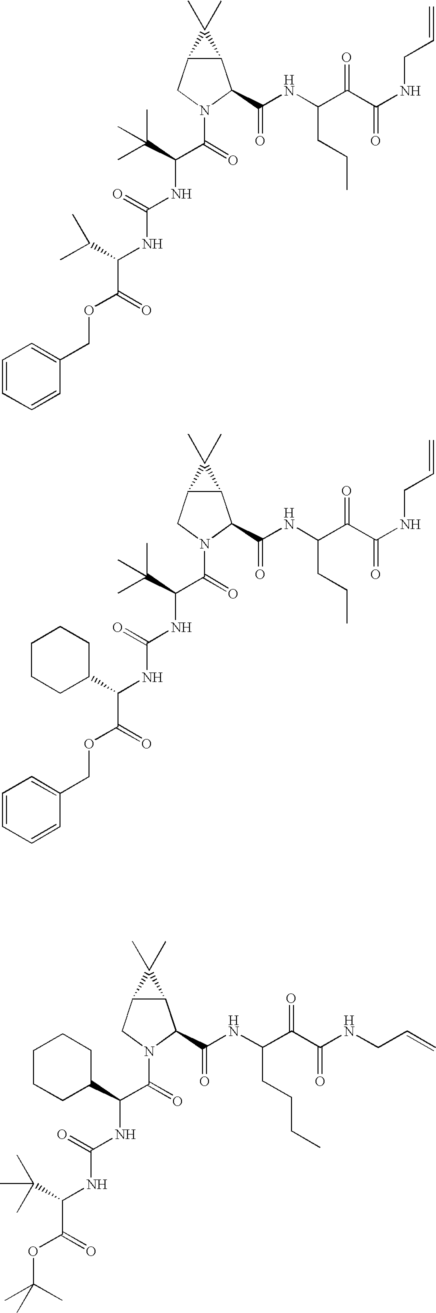

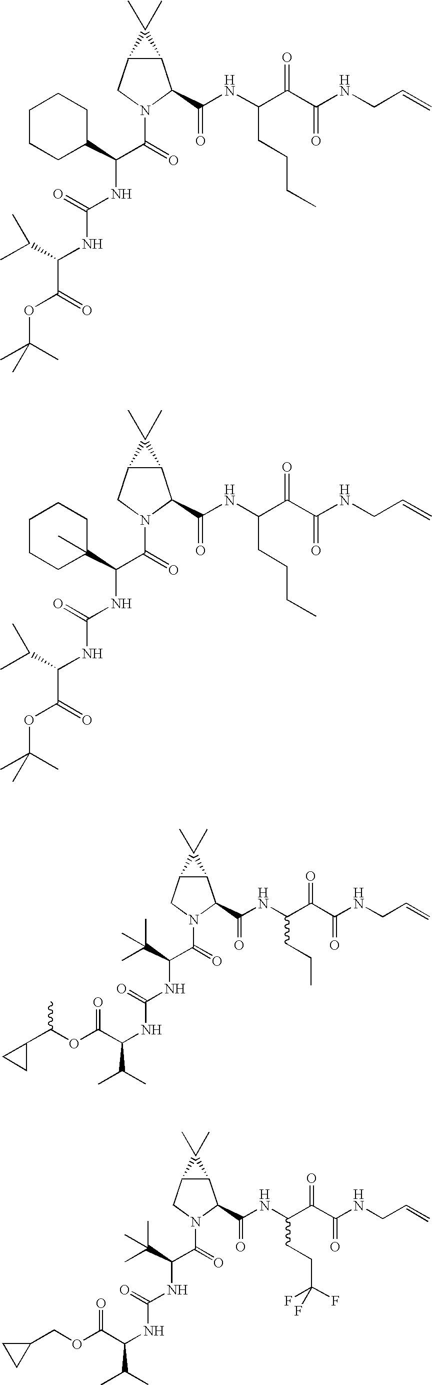

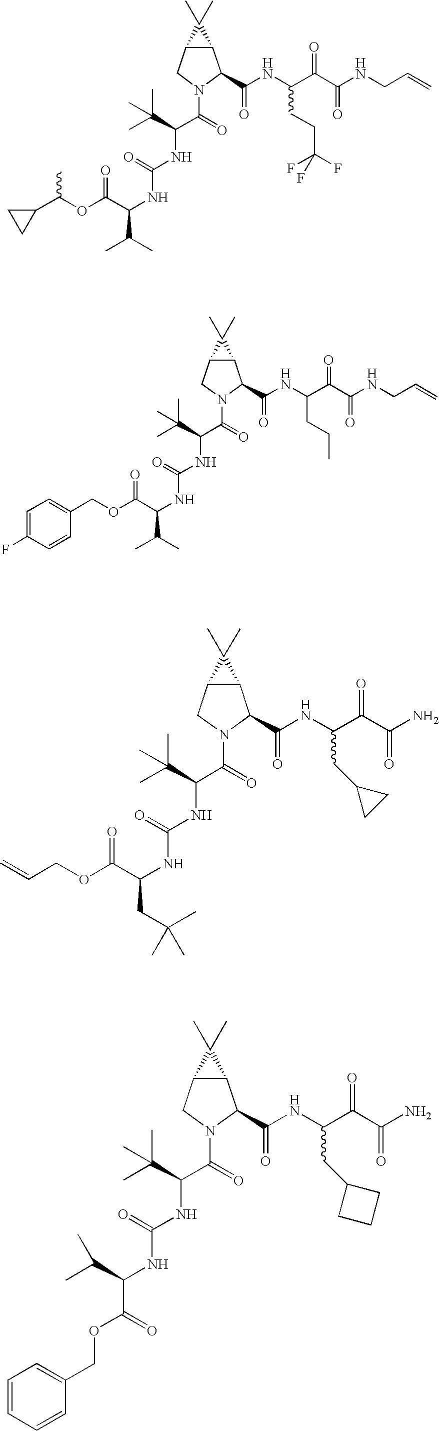

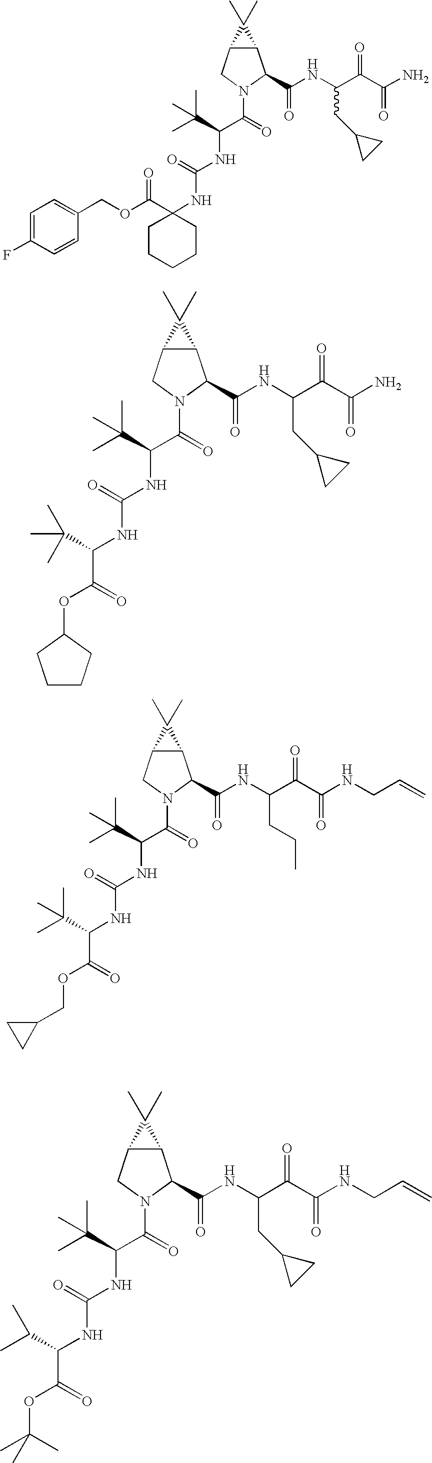

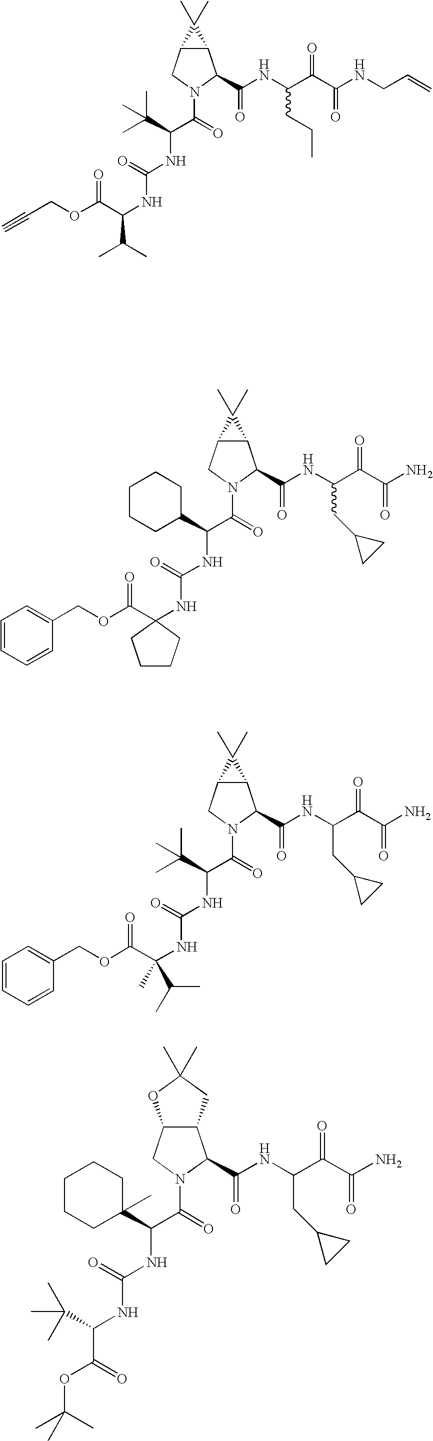

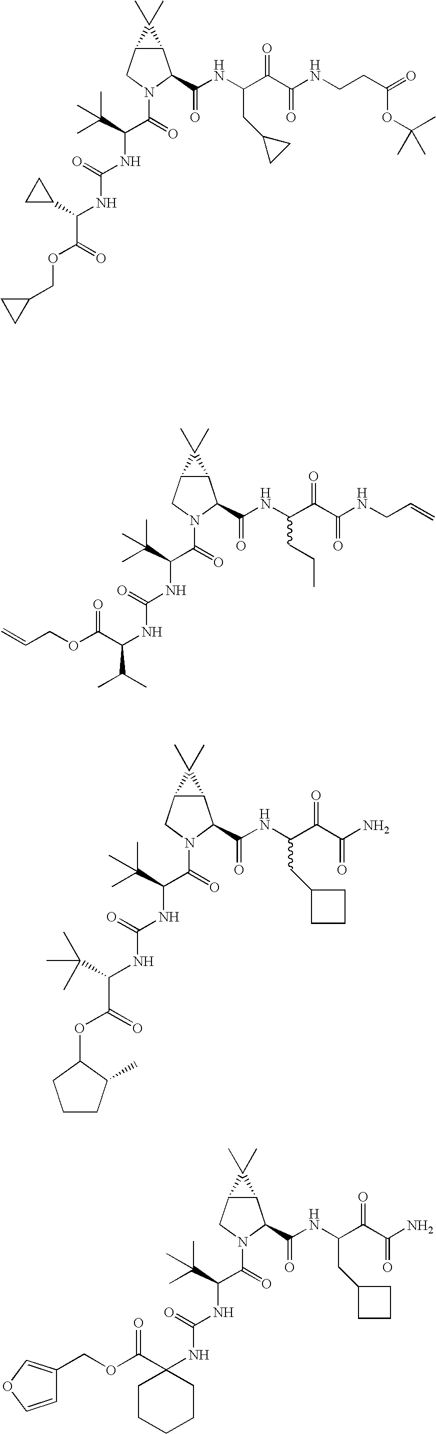

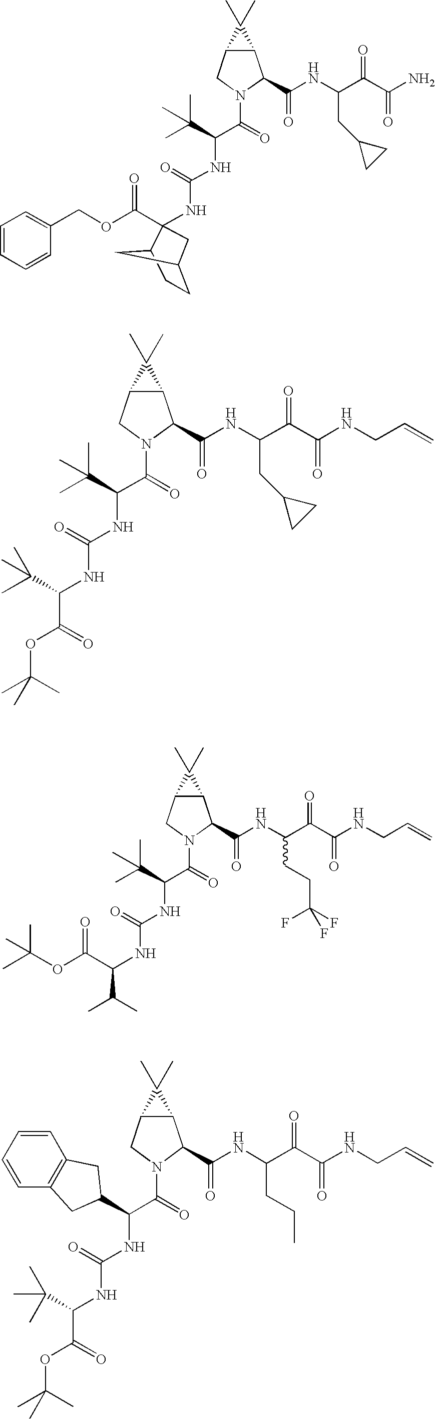

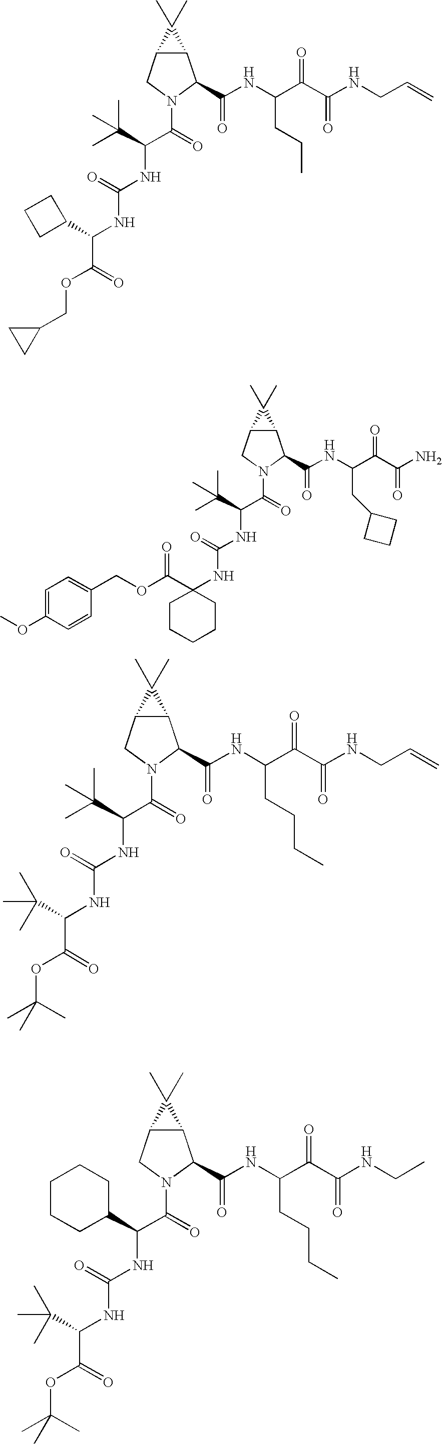

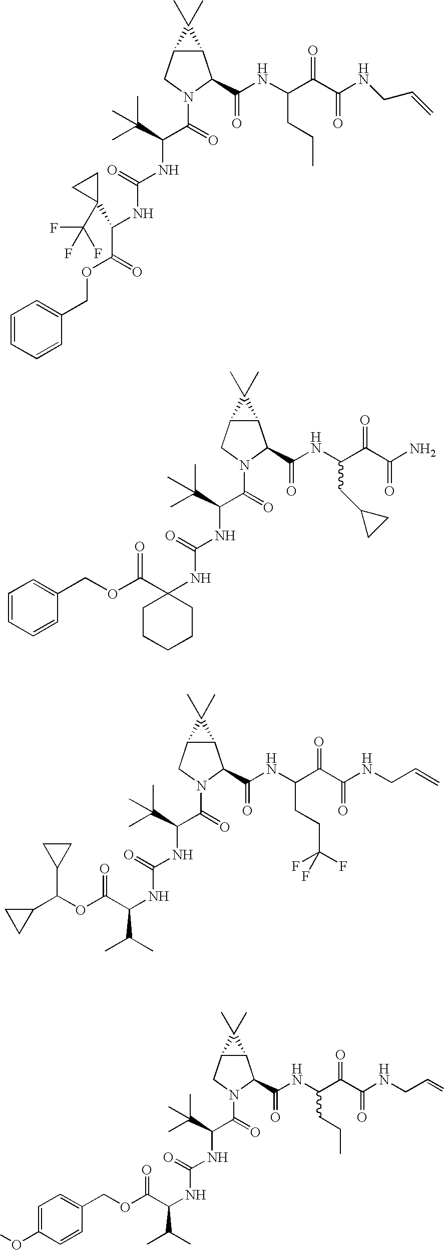

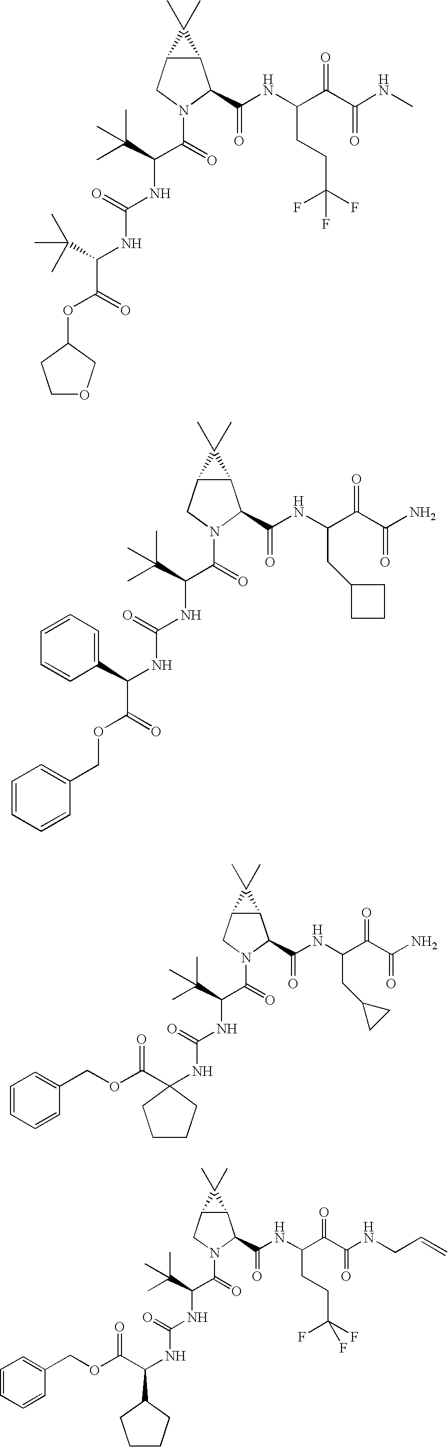

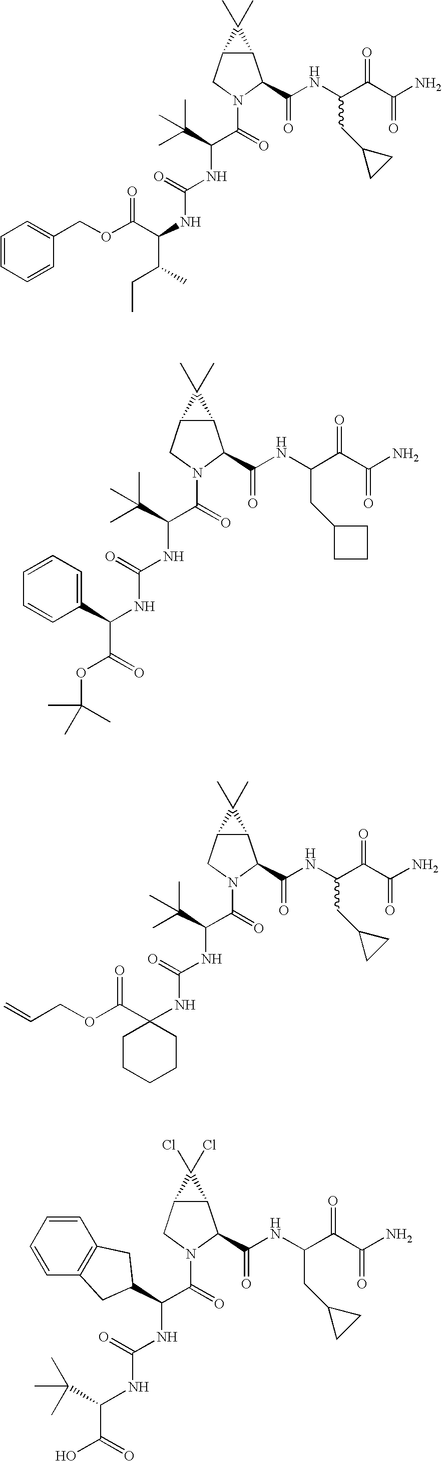

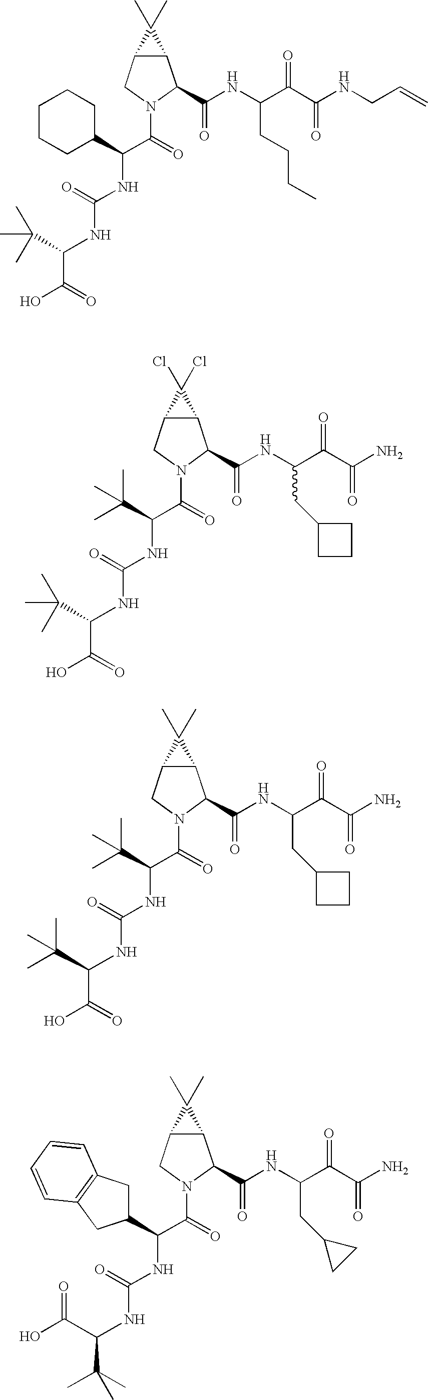

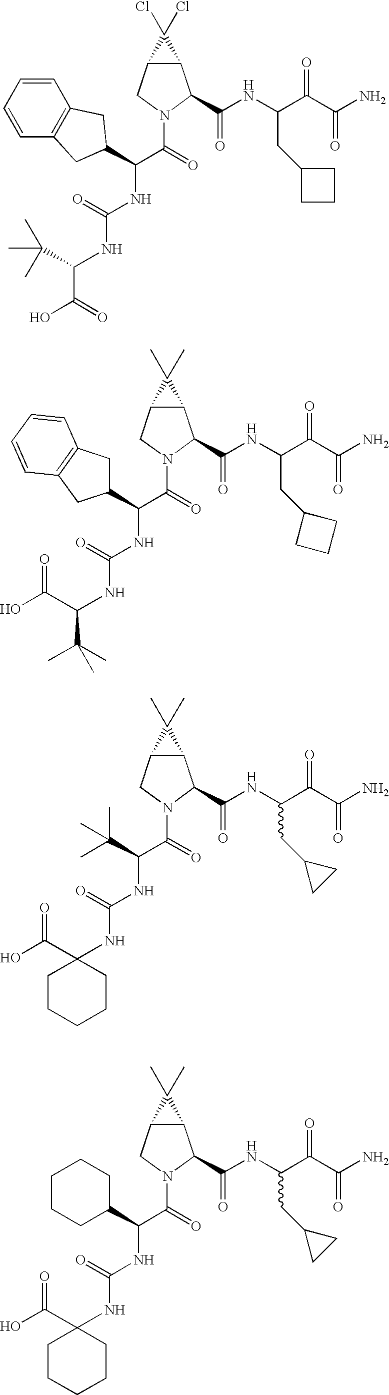

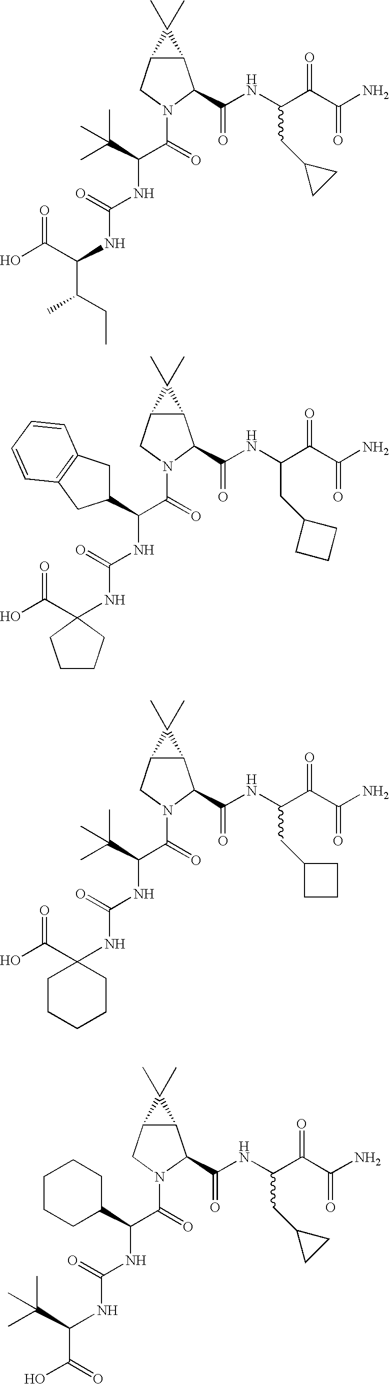

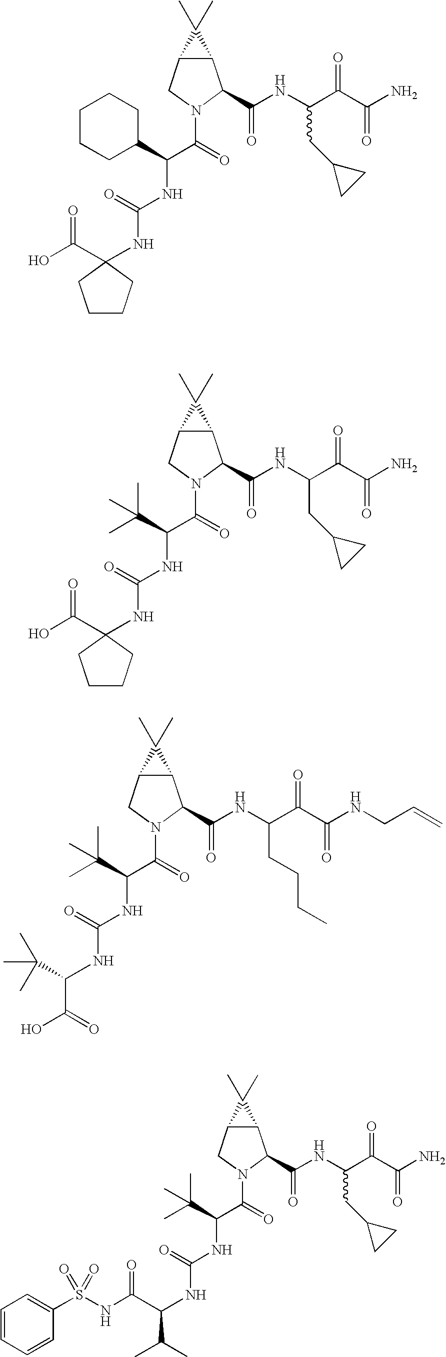

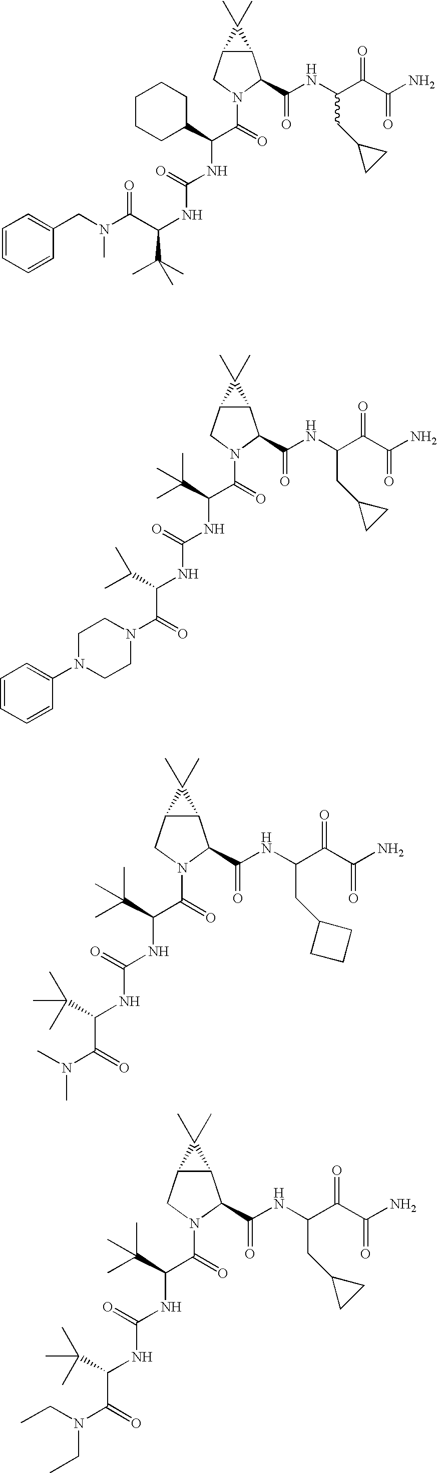

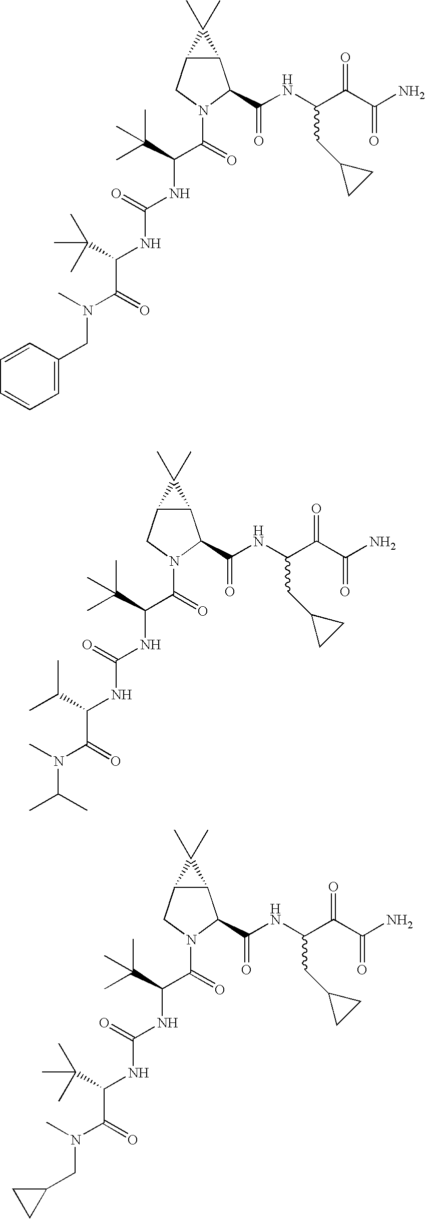

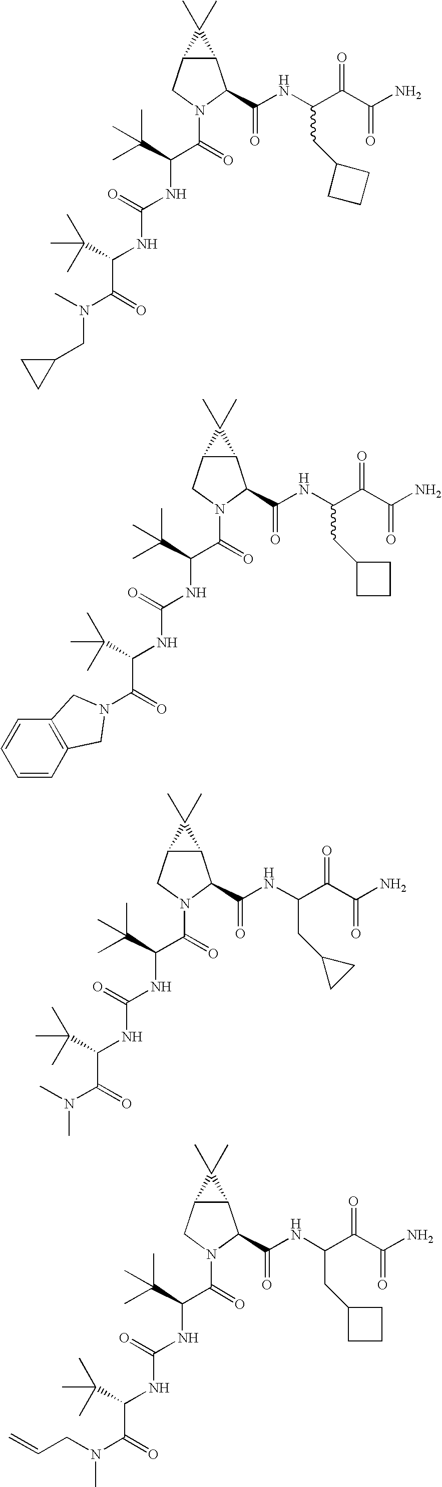

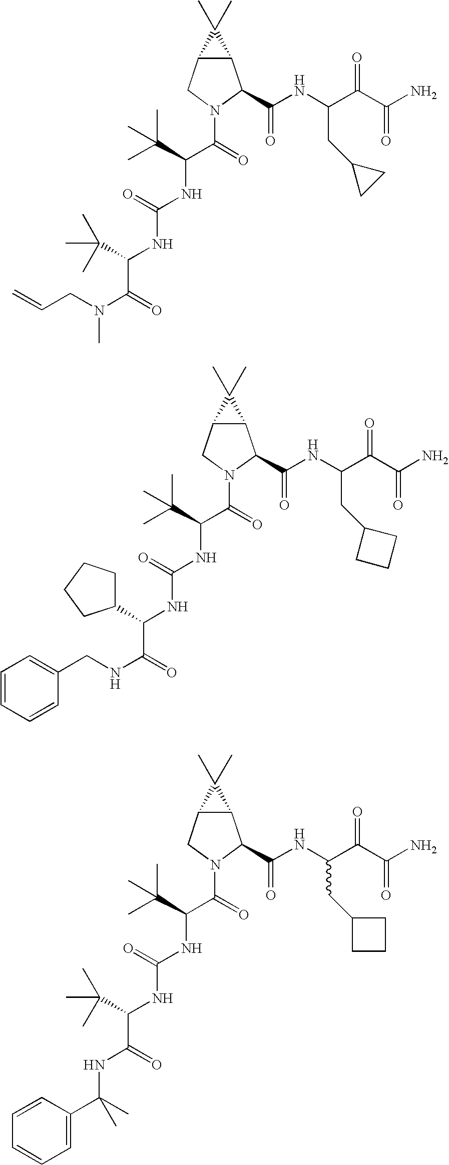

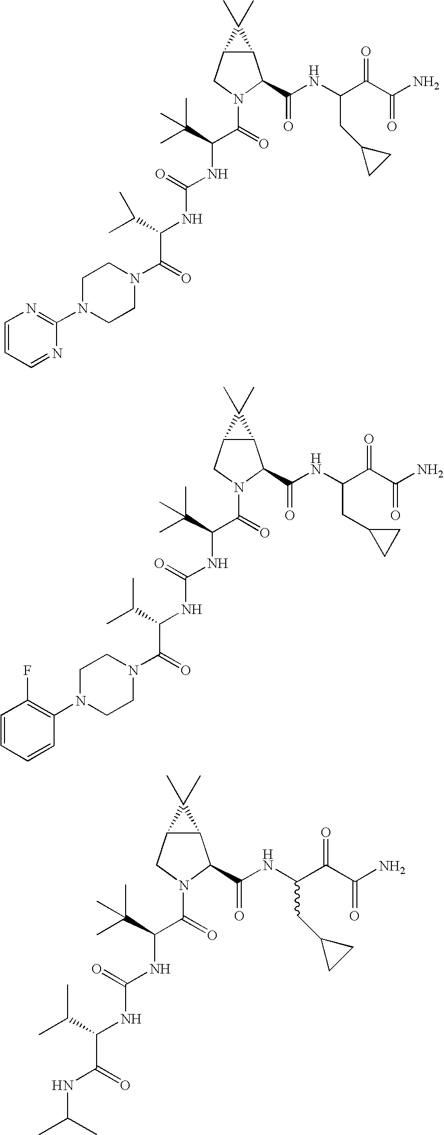

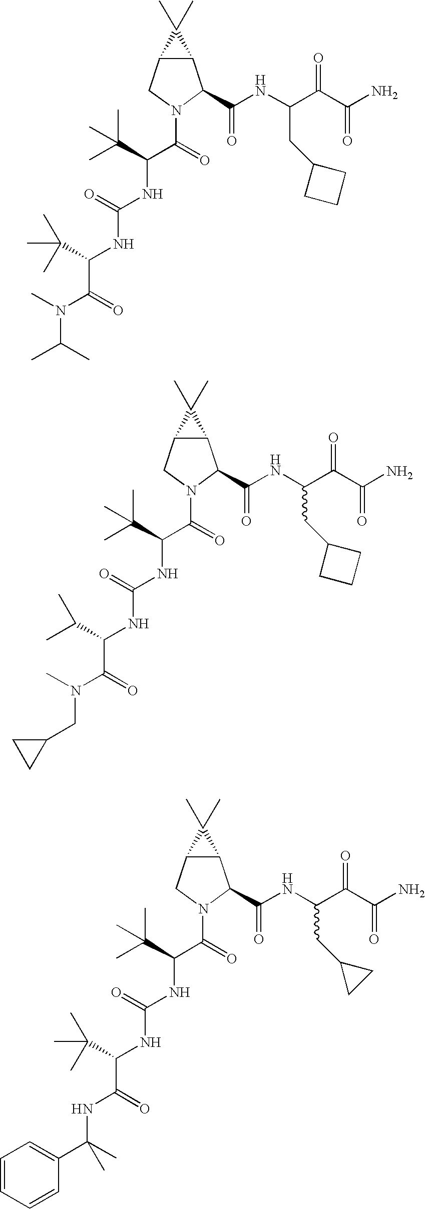

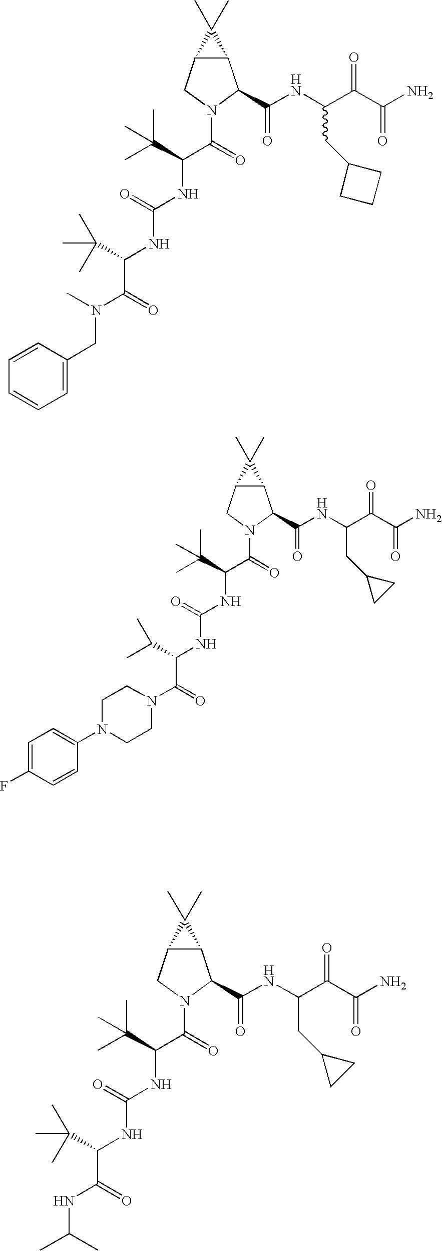

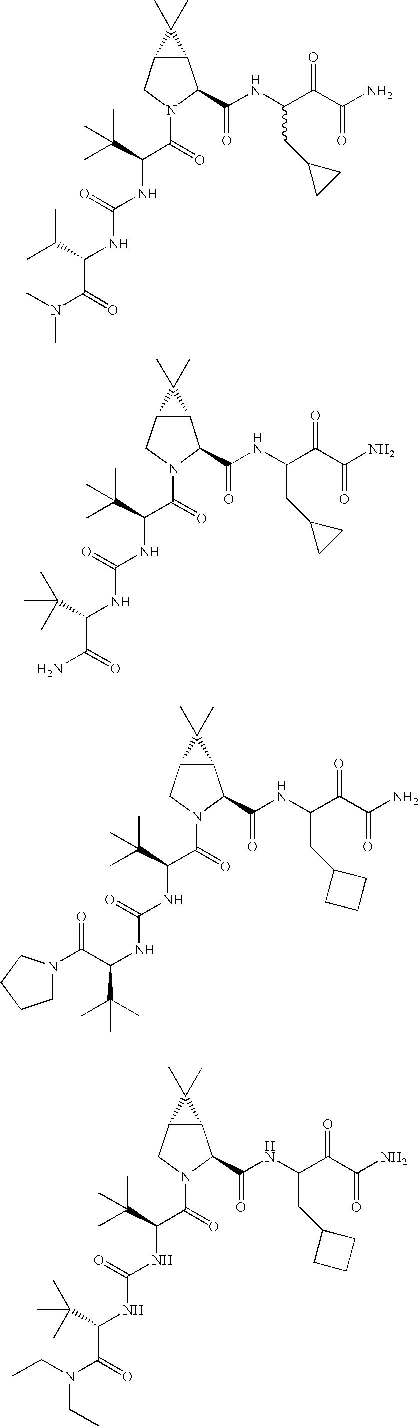

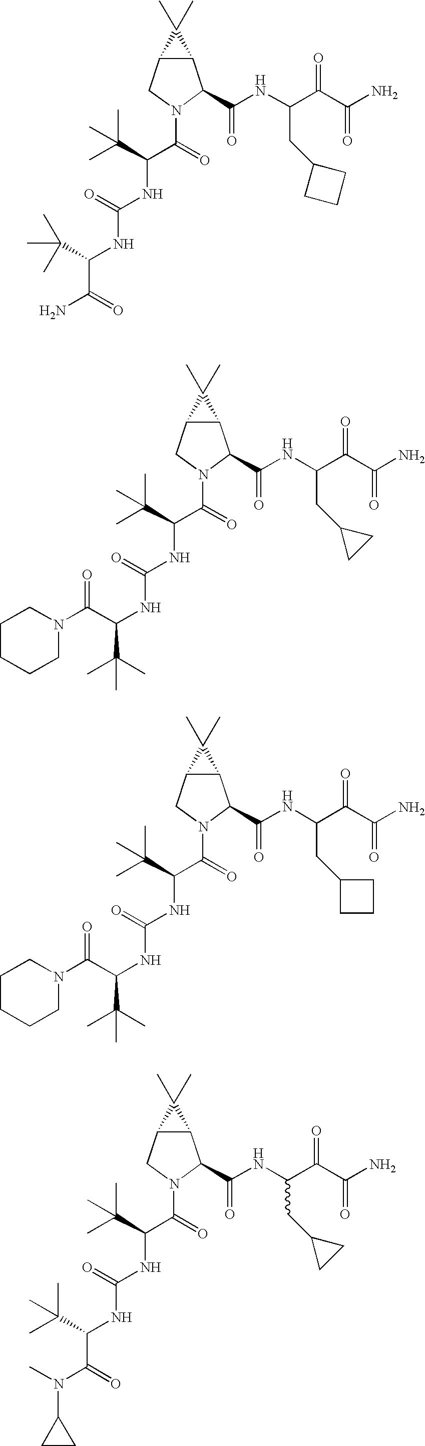

Compounds of formula XI are disclosed in U.S. application Ser. No. 11/065,509 filed Feb. 24, 2005. The preparation of these compounds is disclosed in the experimental section of this application set forth hereinbelow.

Non-limiting examples of certain compounds disclosed in U.S. application Ser. No. 11/065,509 are: 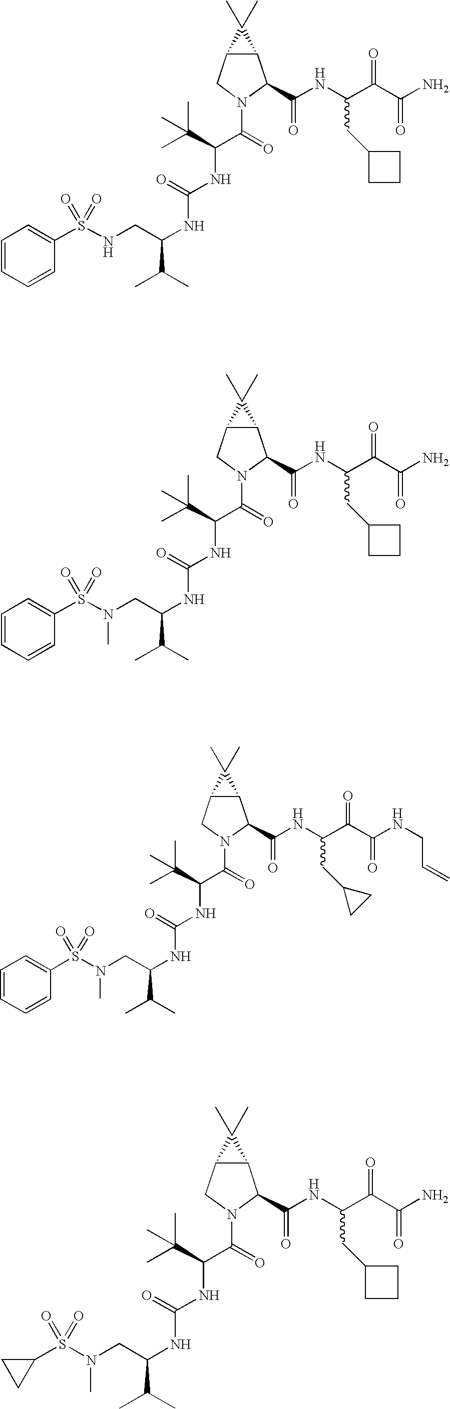 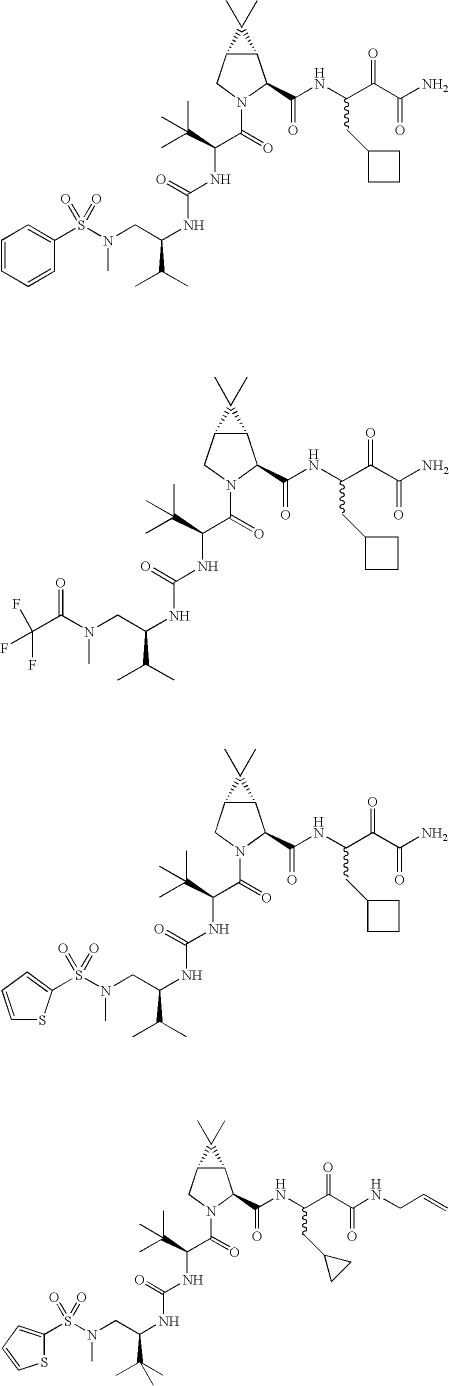 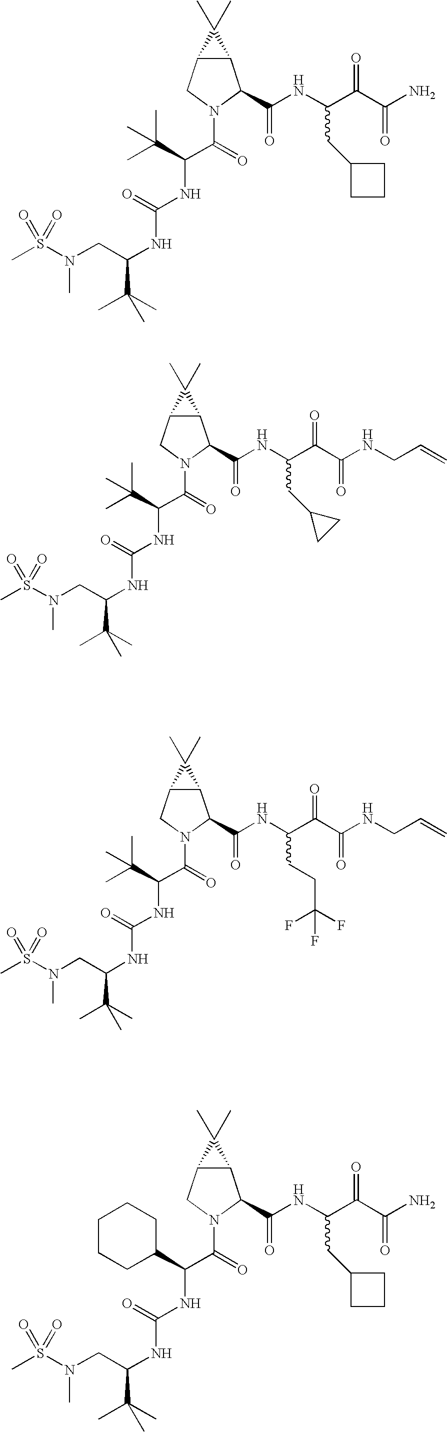 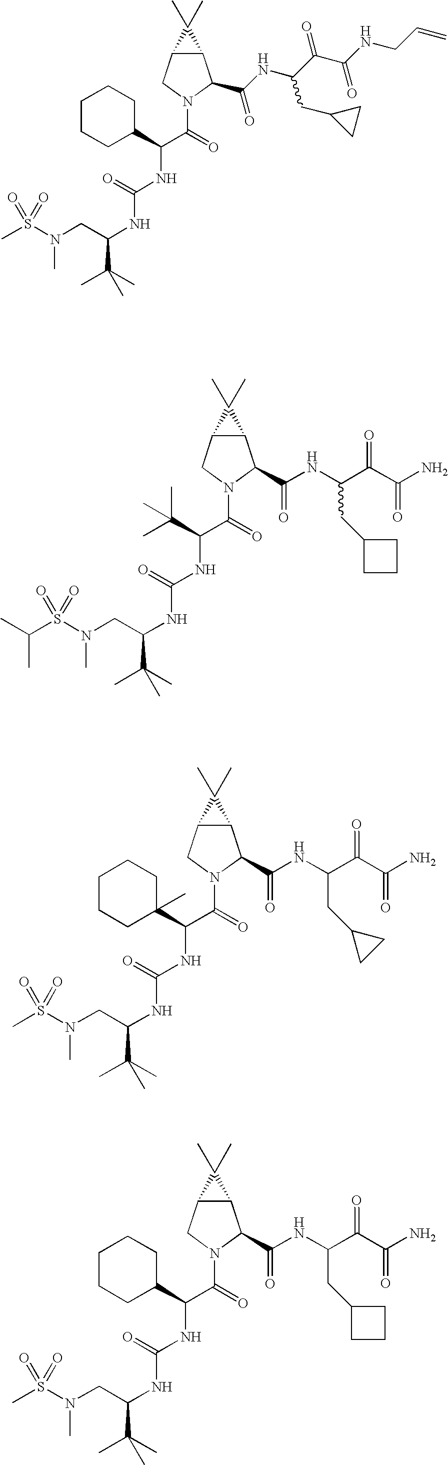 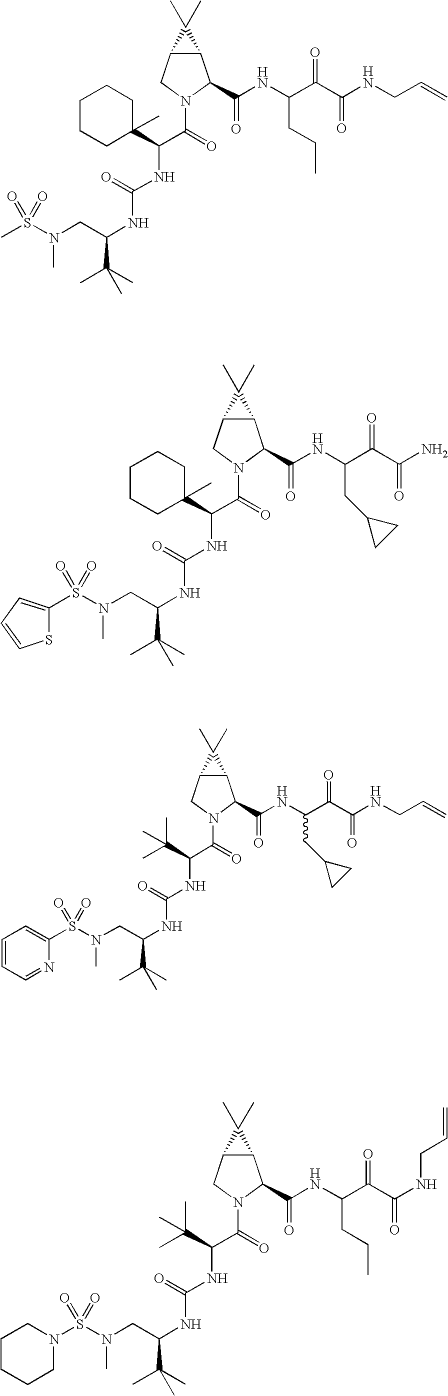 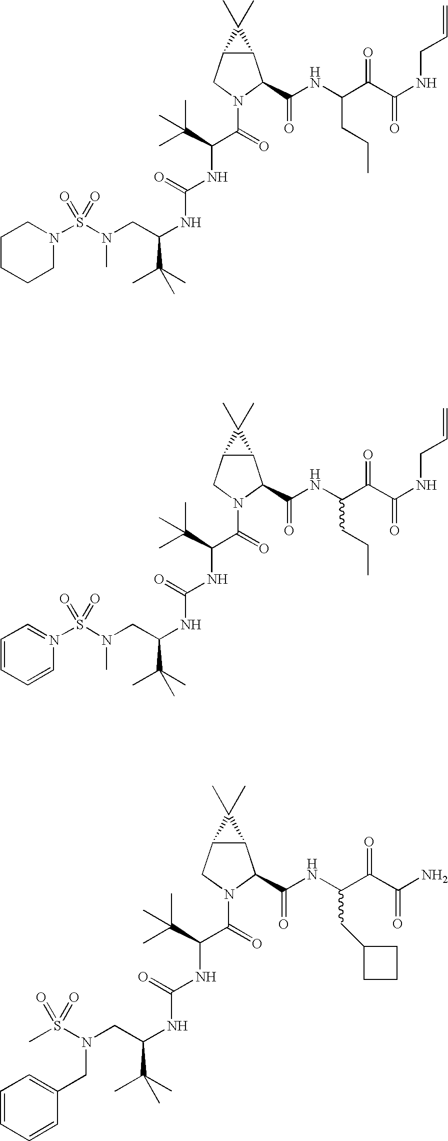 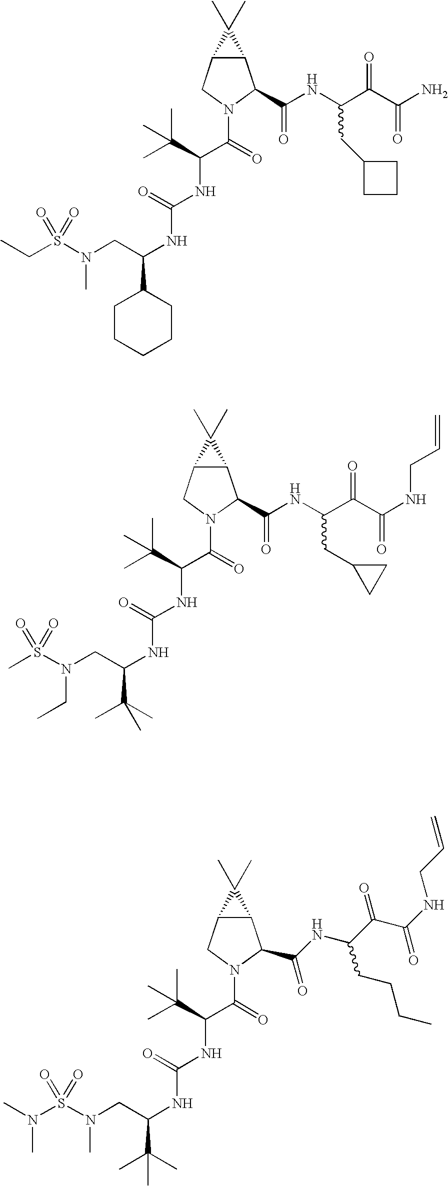 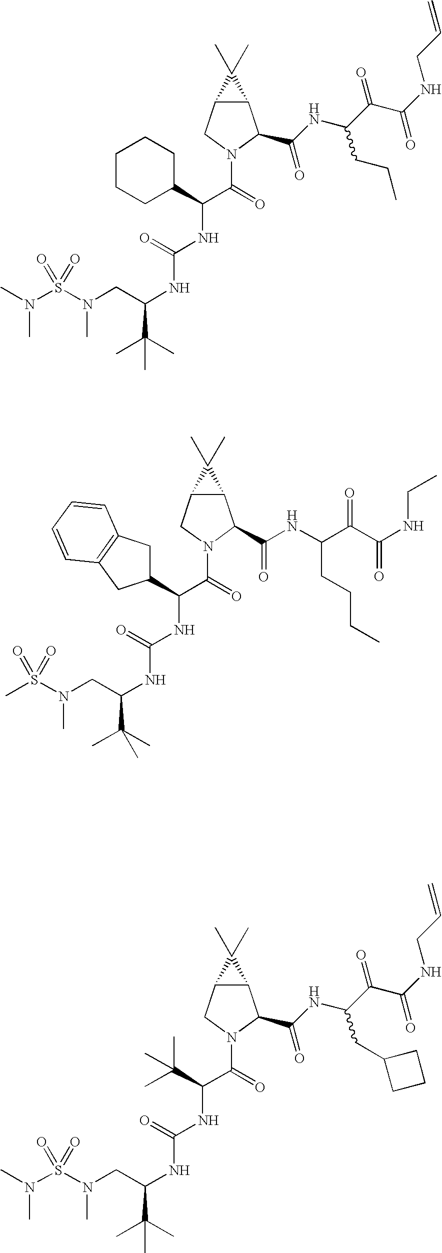 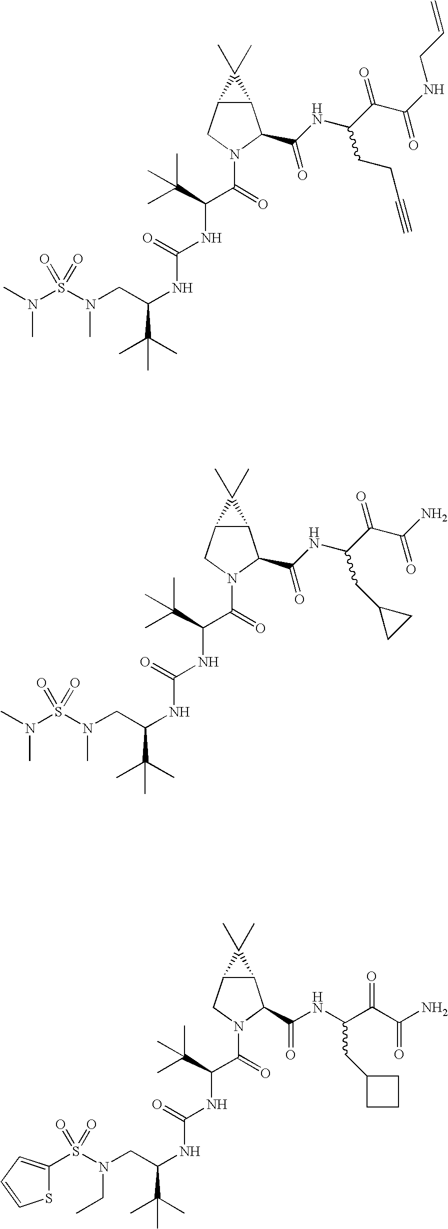 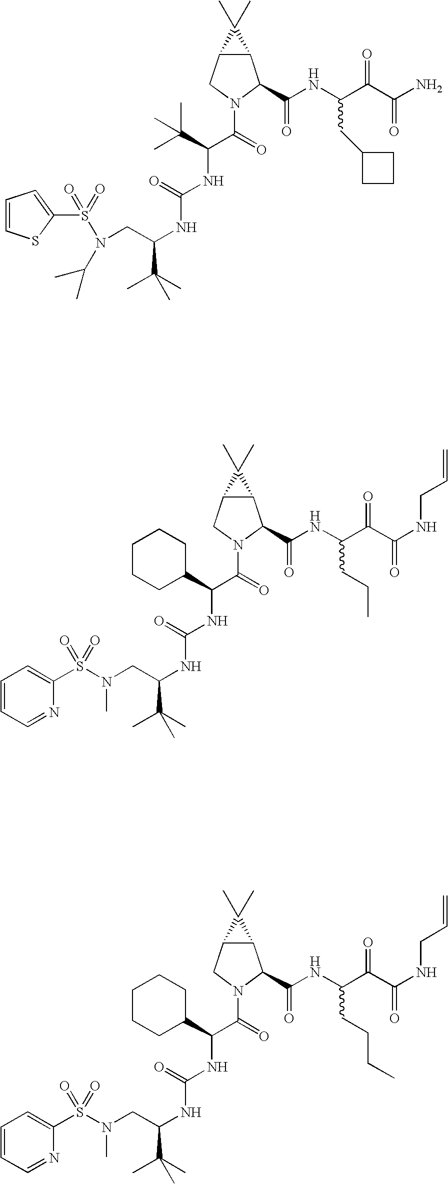 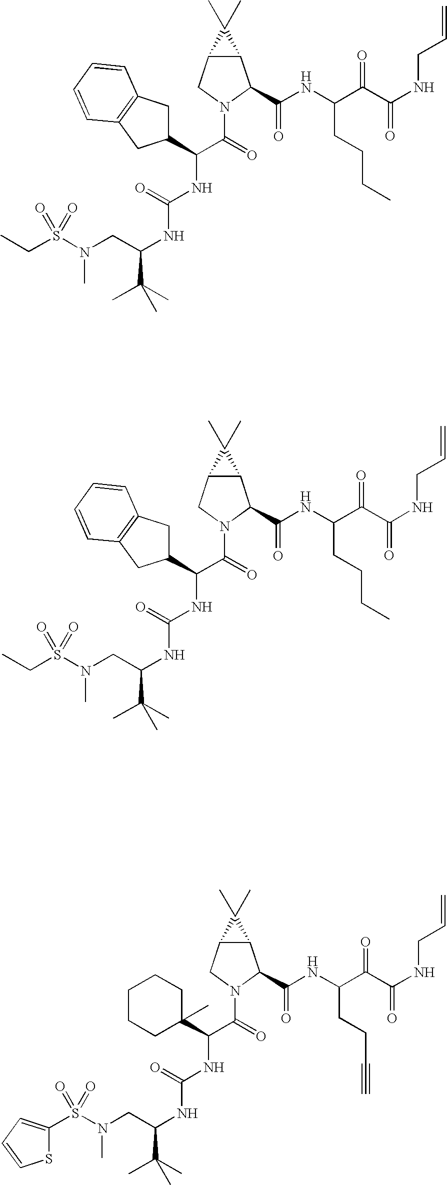 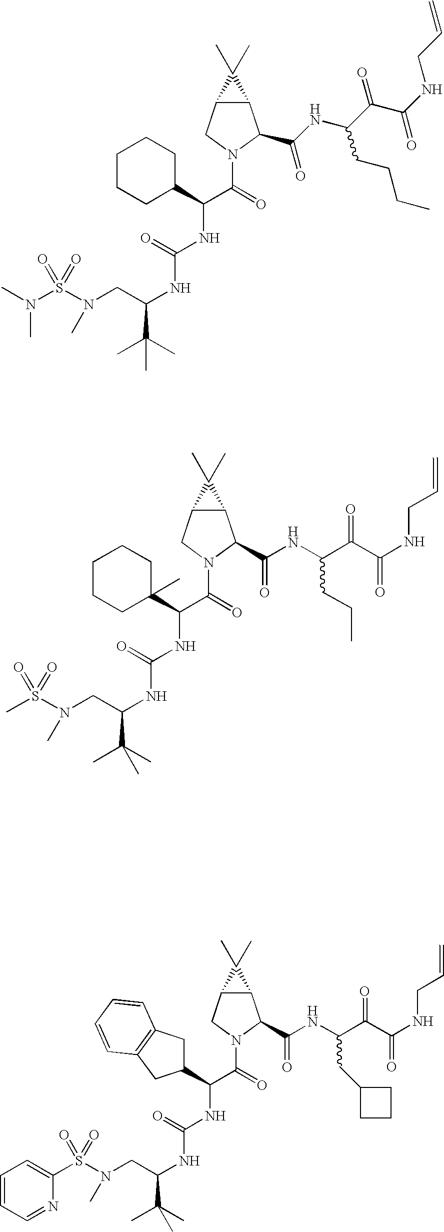 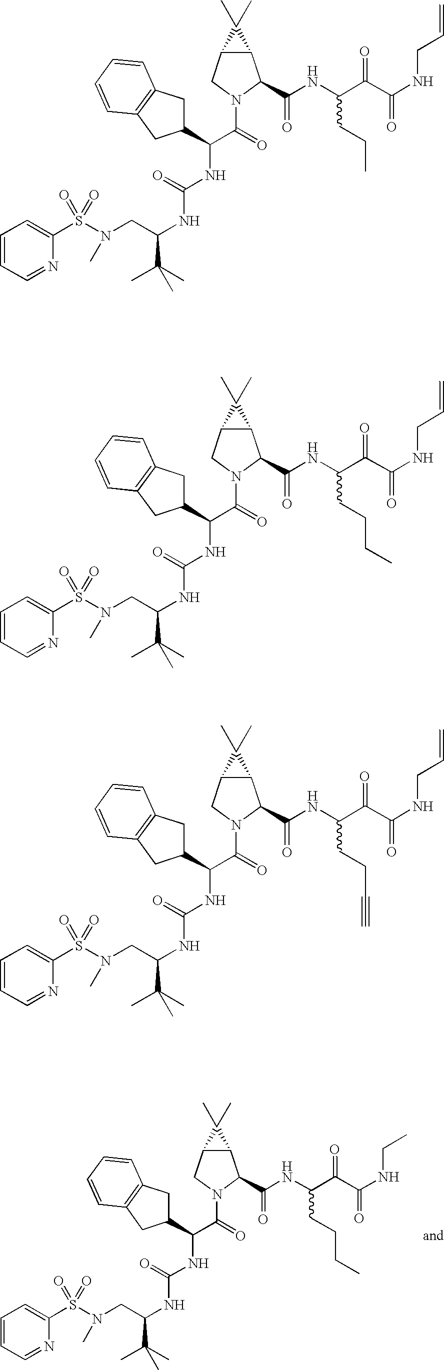 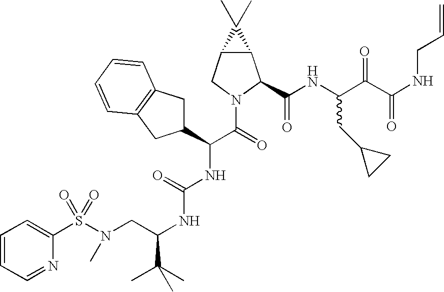
or a pharmaceutically acceptable salt, solvate or ester thereof.

Compounds of formula XII are disclosed in U.S. patent application Ser. No. 11/065,531 filed Feb. 24, 2005. The preparation of these compounds is disclosed in the experimental section of this application set forth hereinbelow.

Non-limiting examples of certain compounds disclosed in U.S. patent application Ser. No. 11/065,531 are: 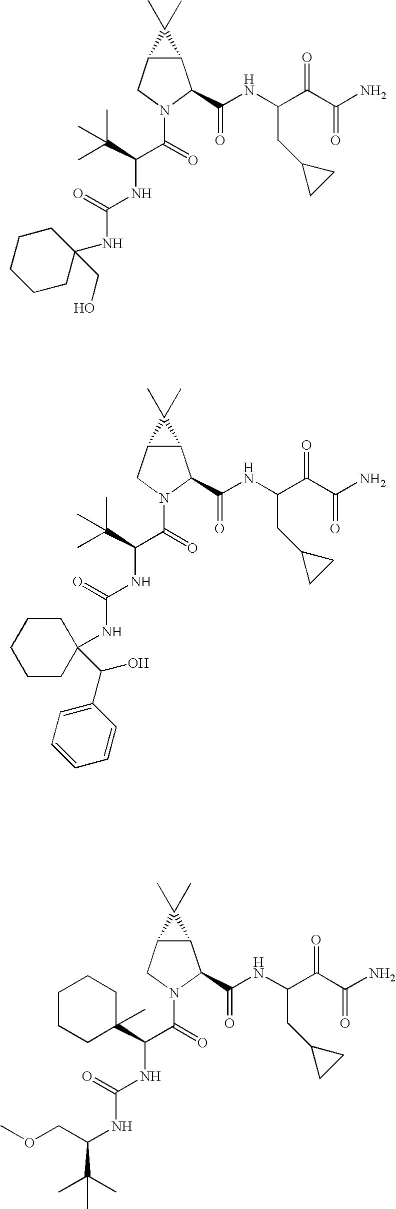 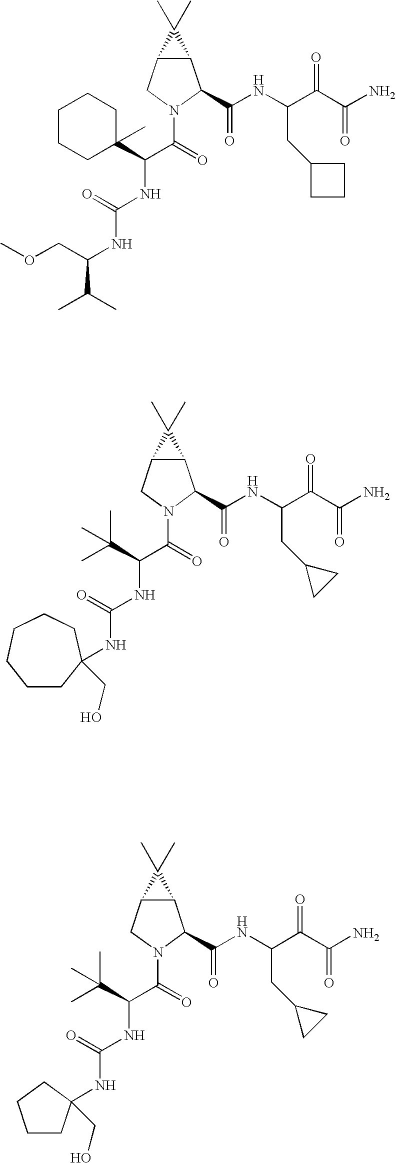 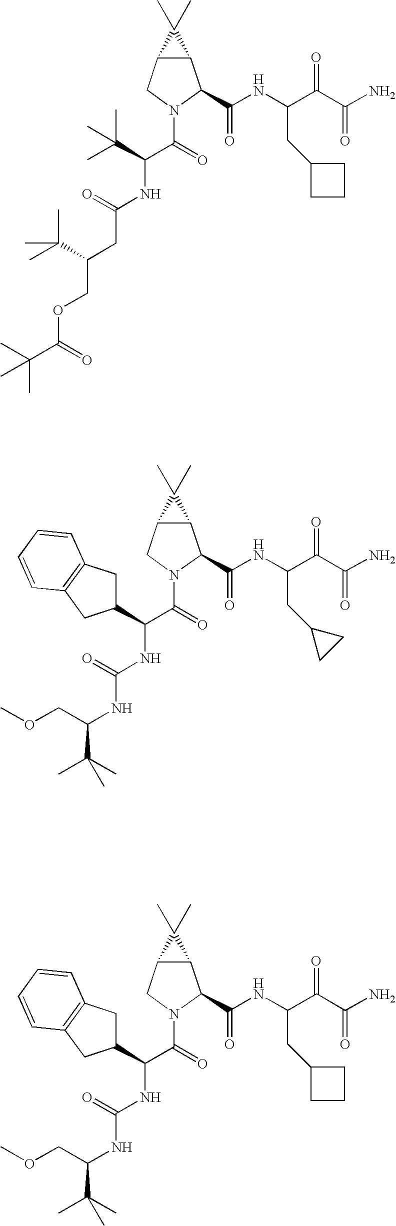 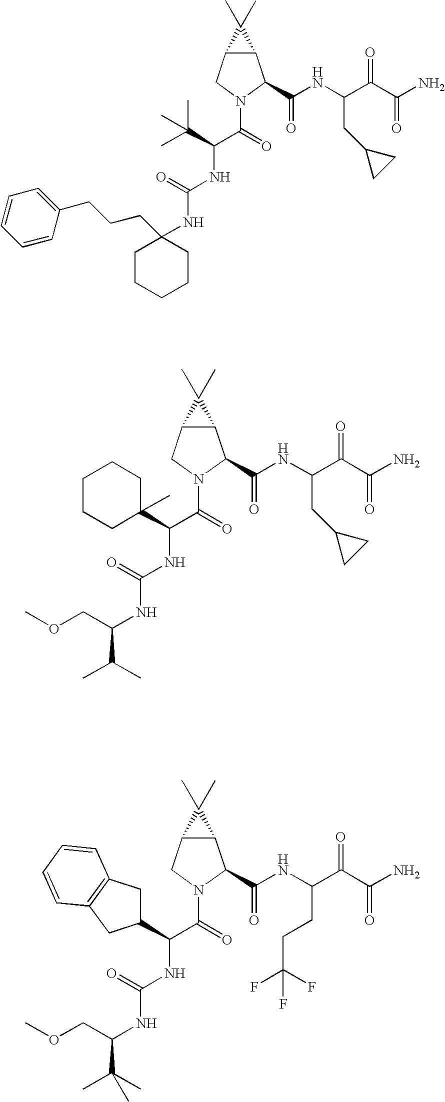 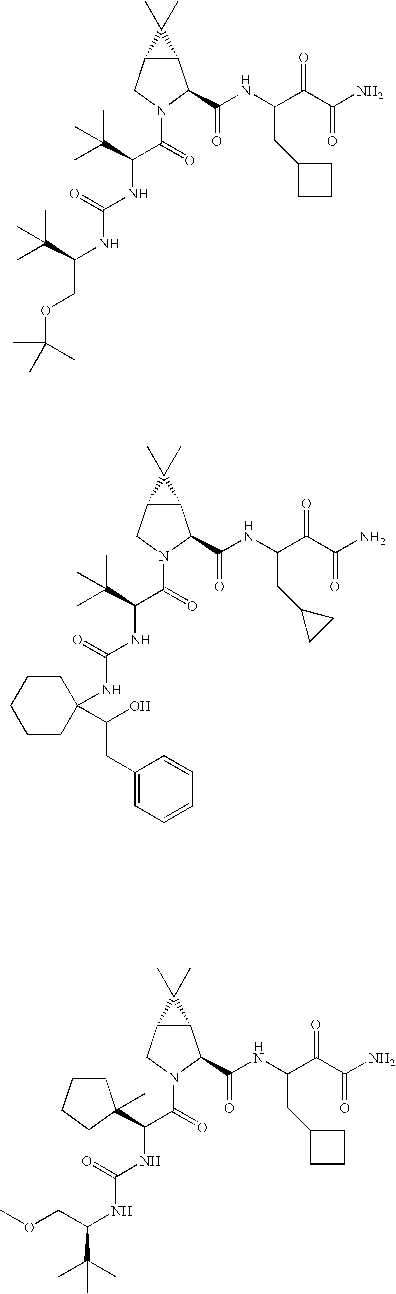 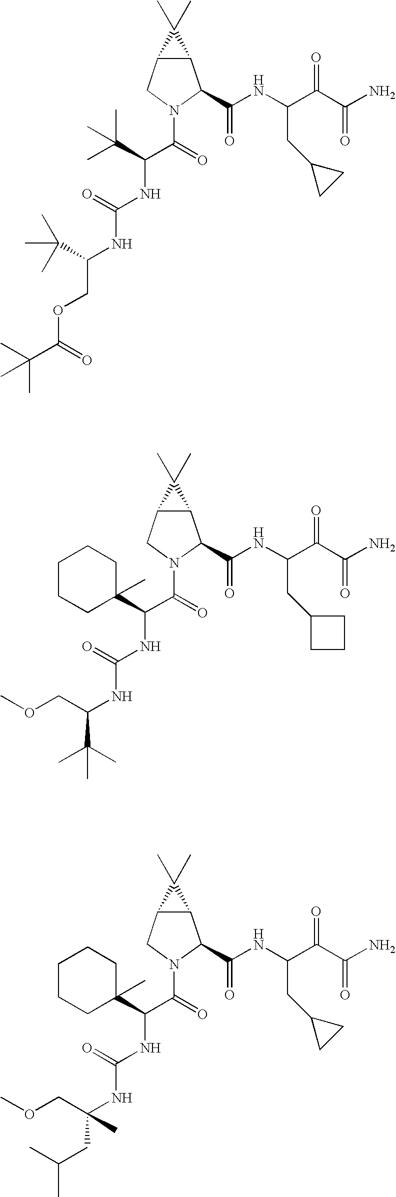 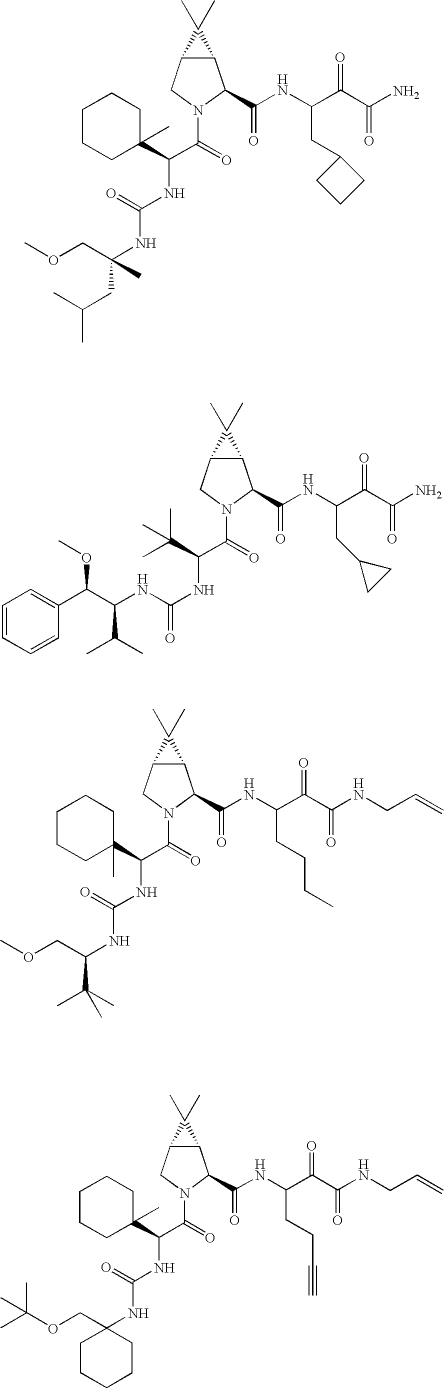 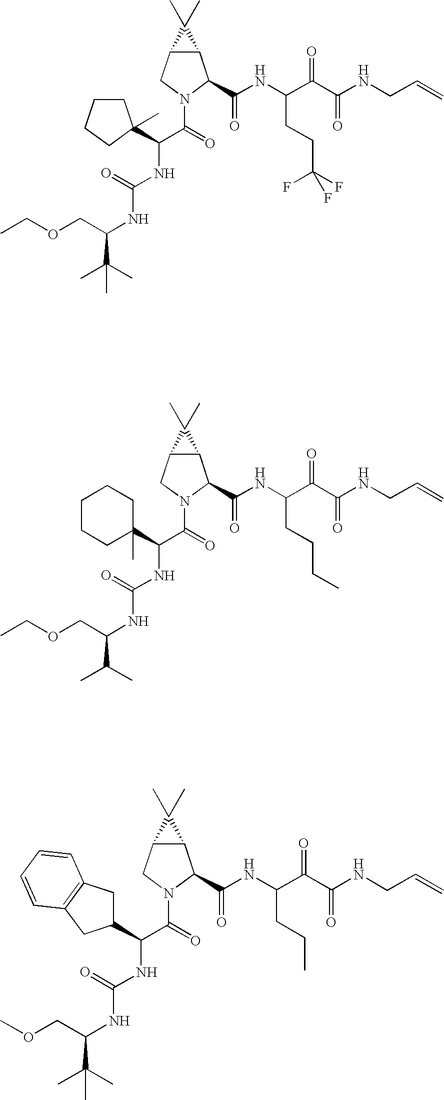 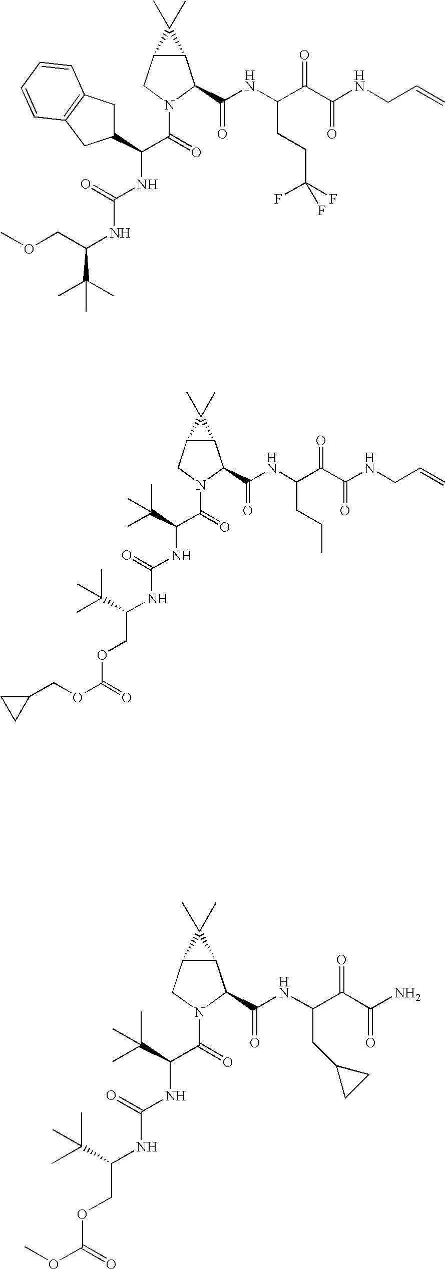 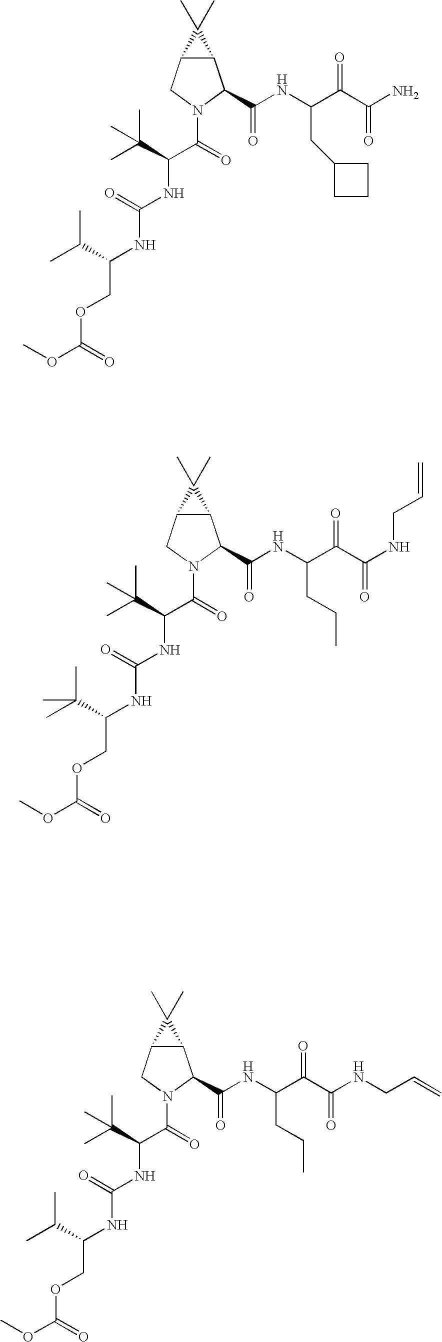 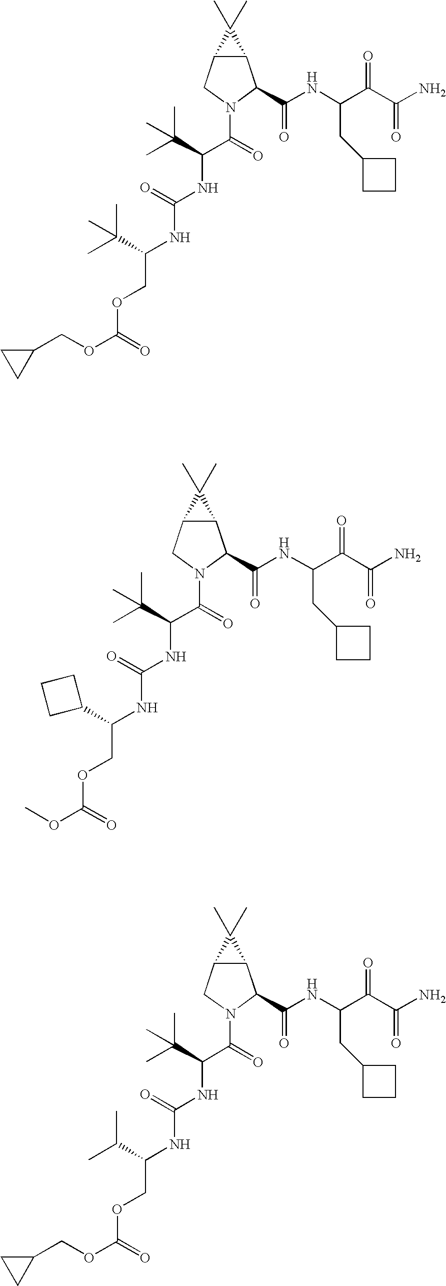 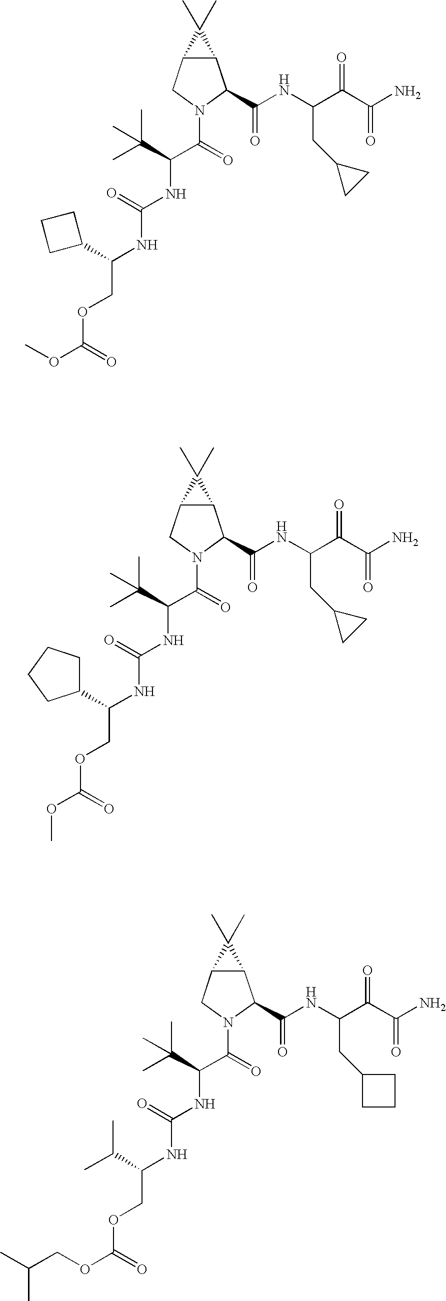 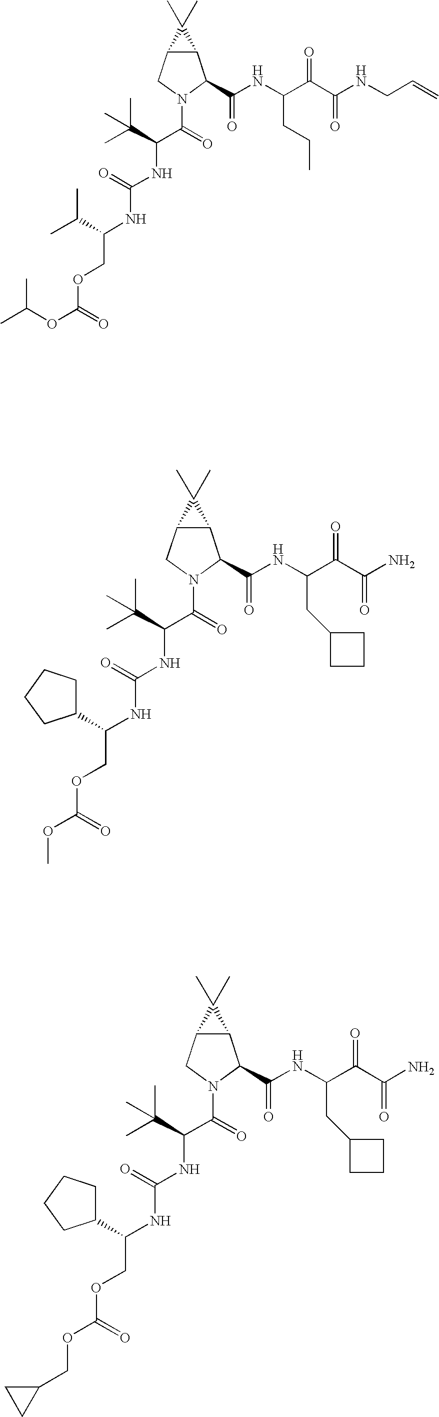 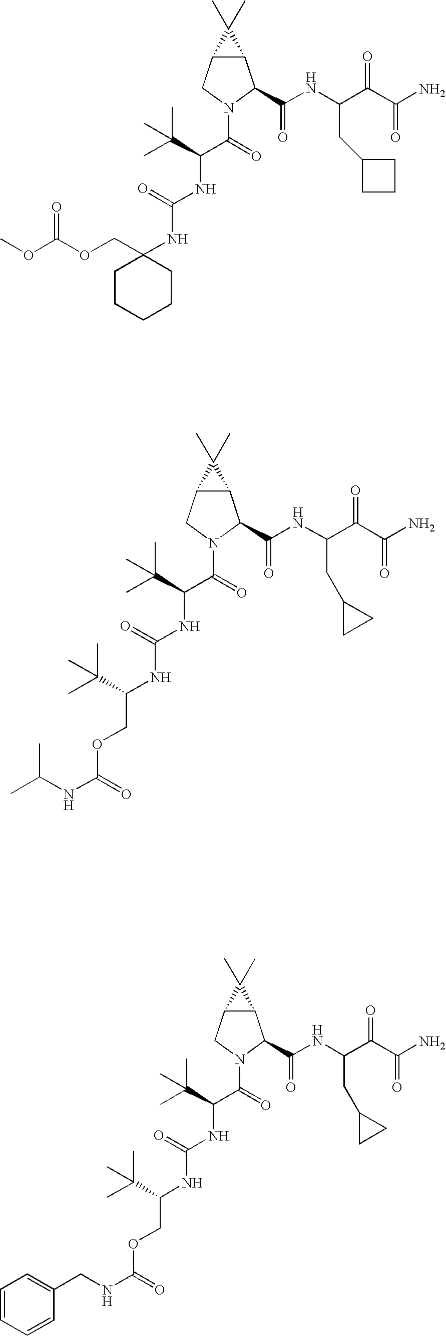 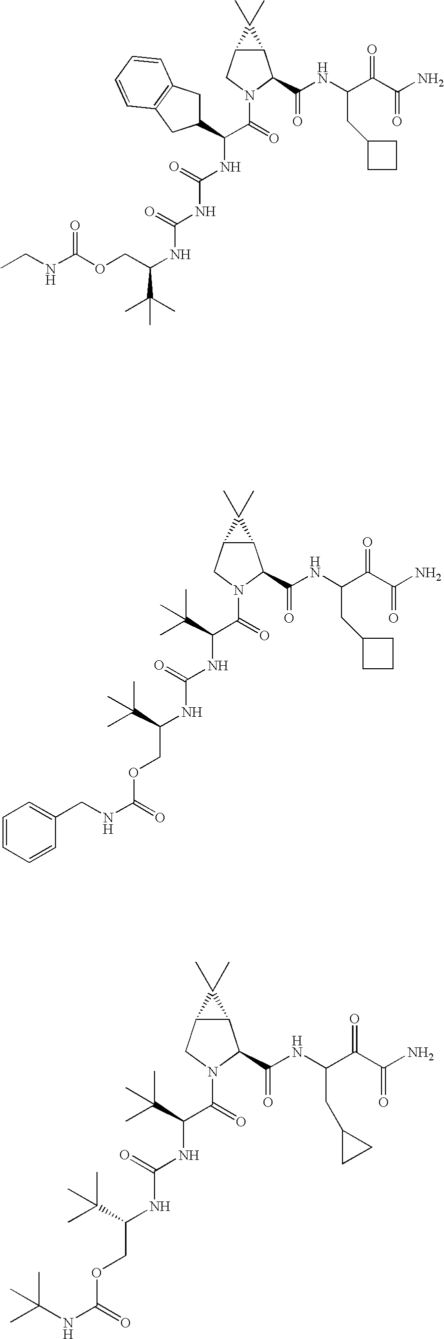 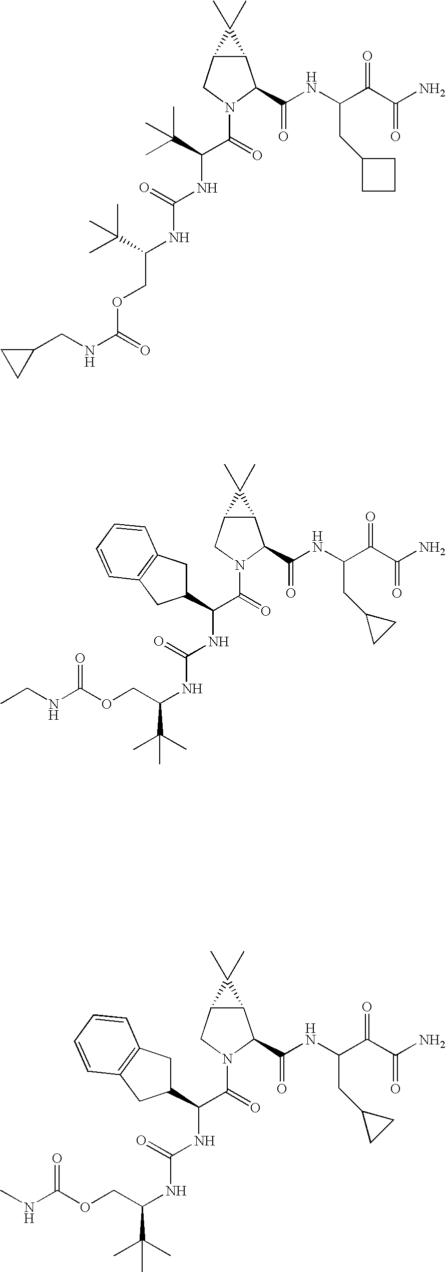 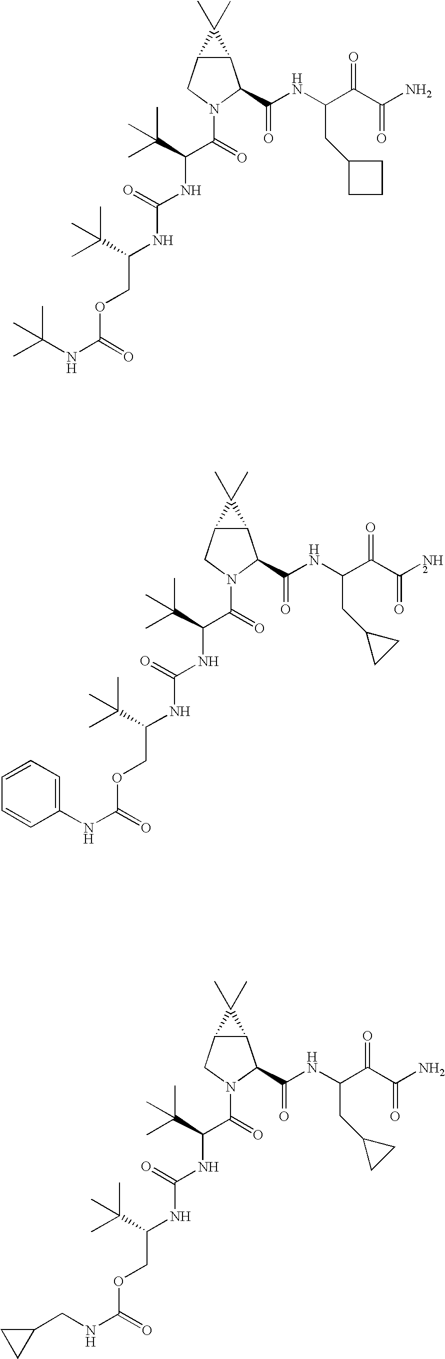 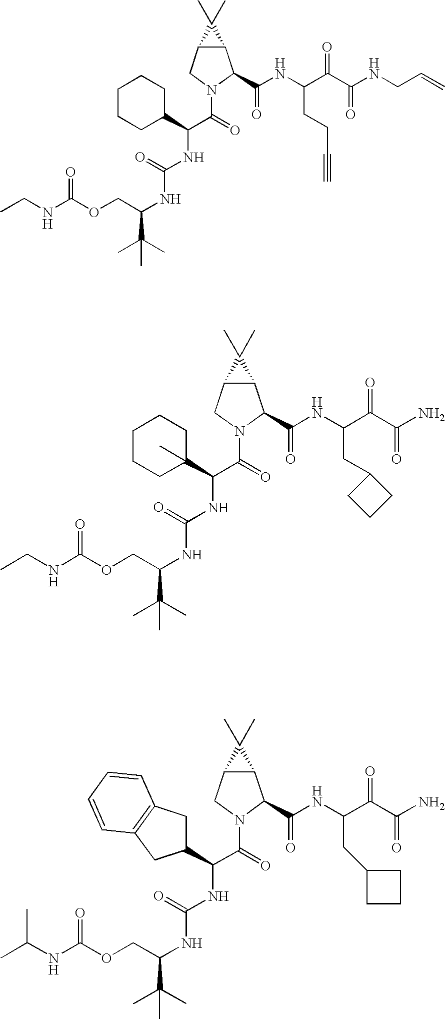 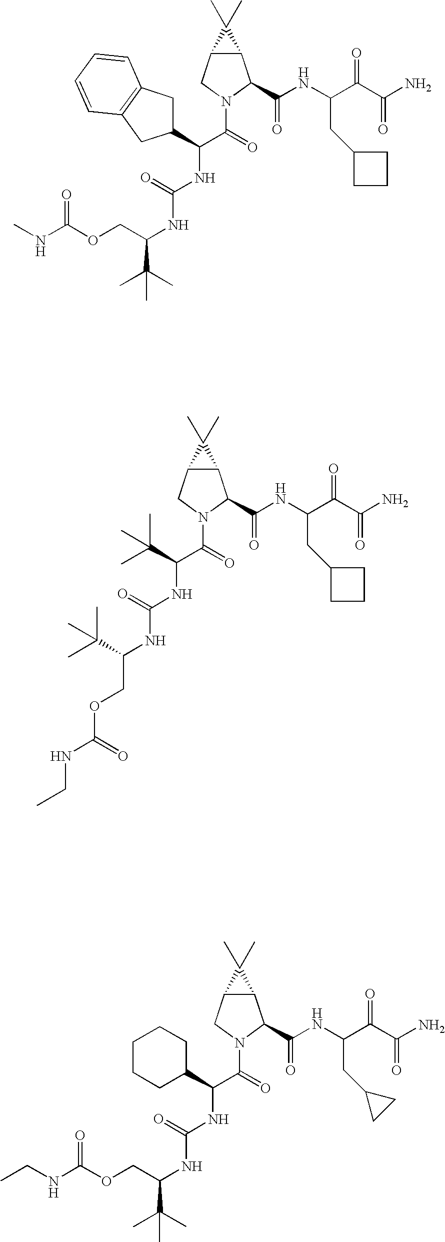 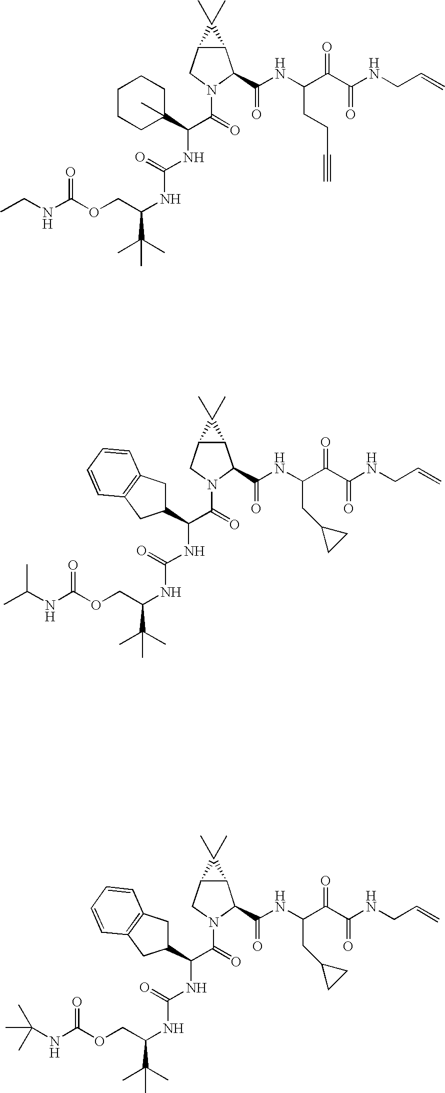 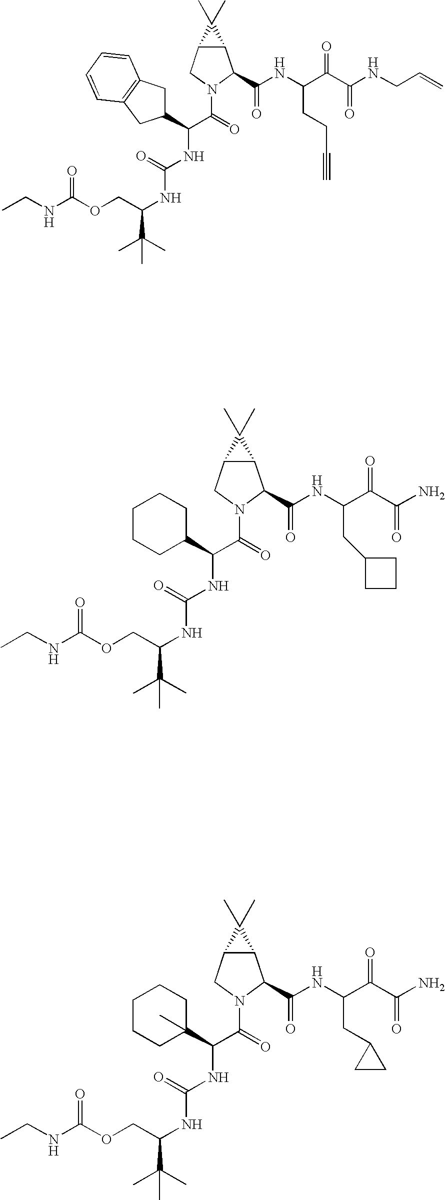 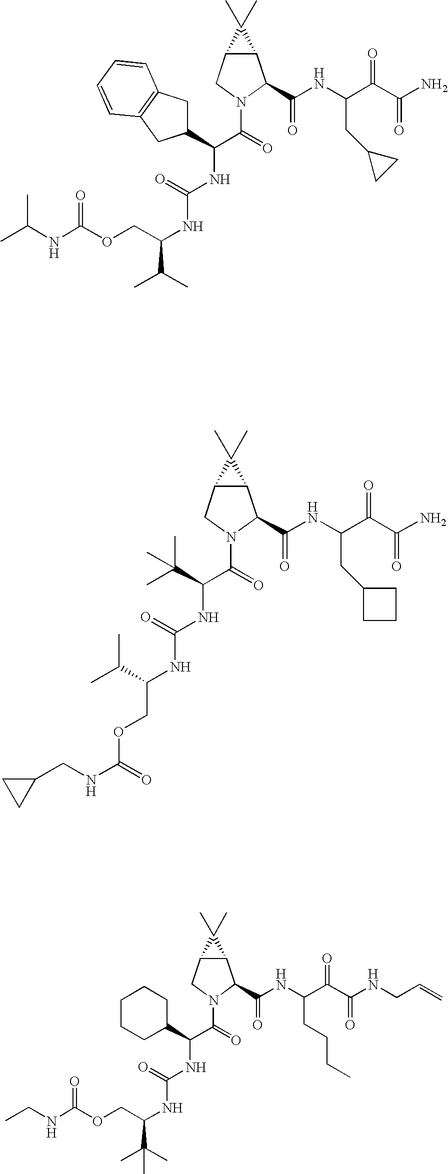 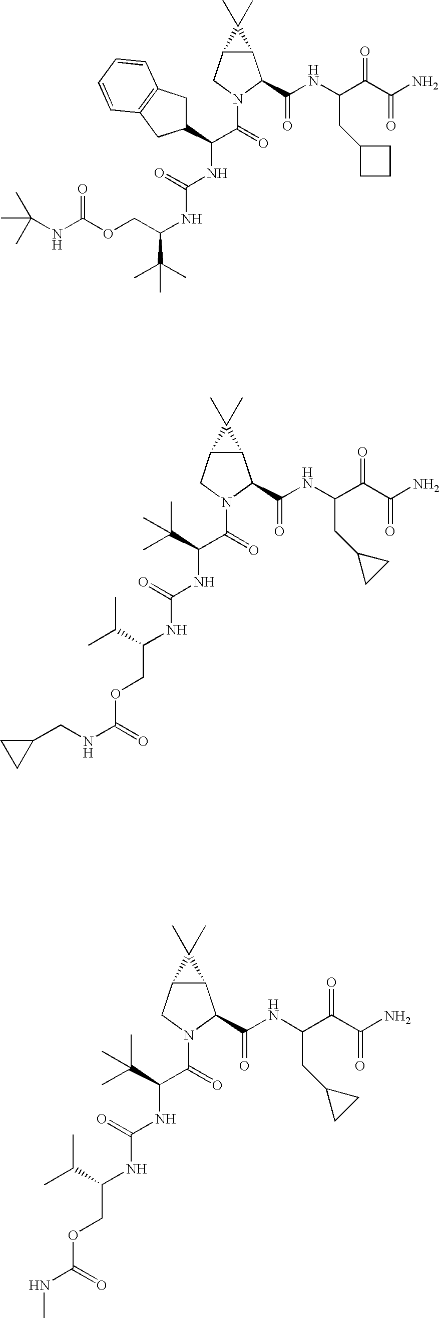 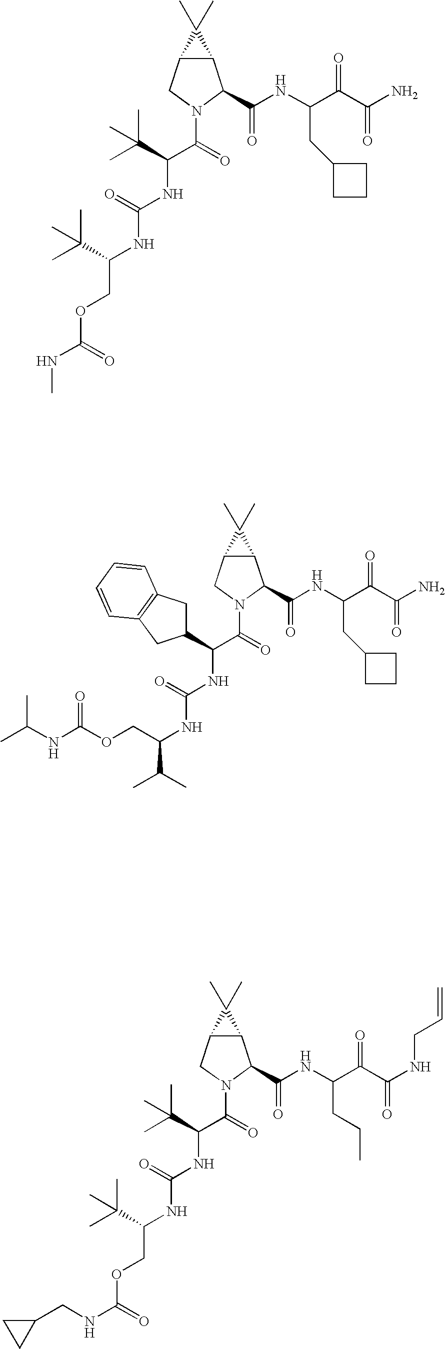 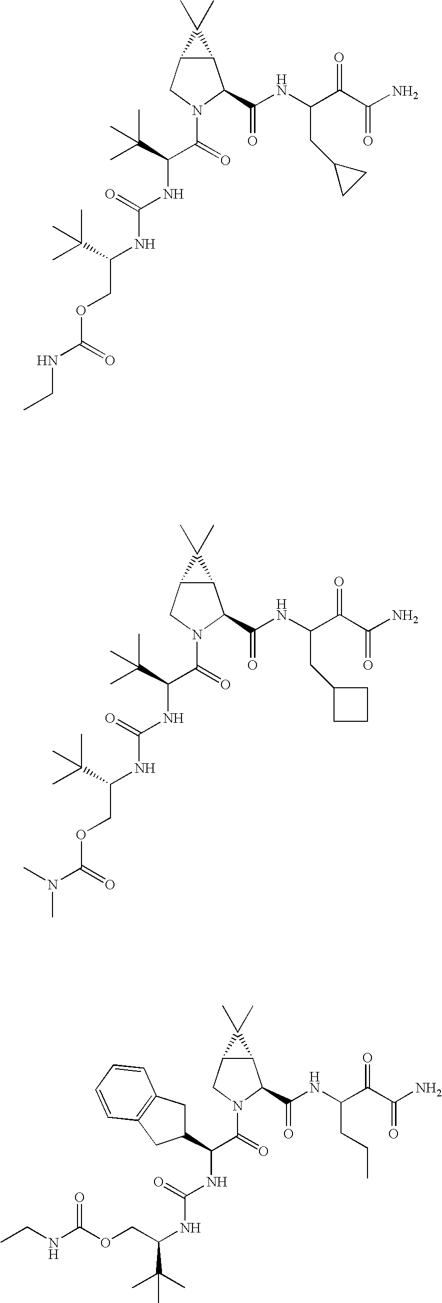 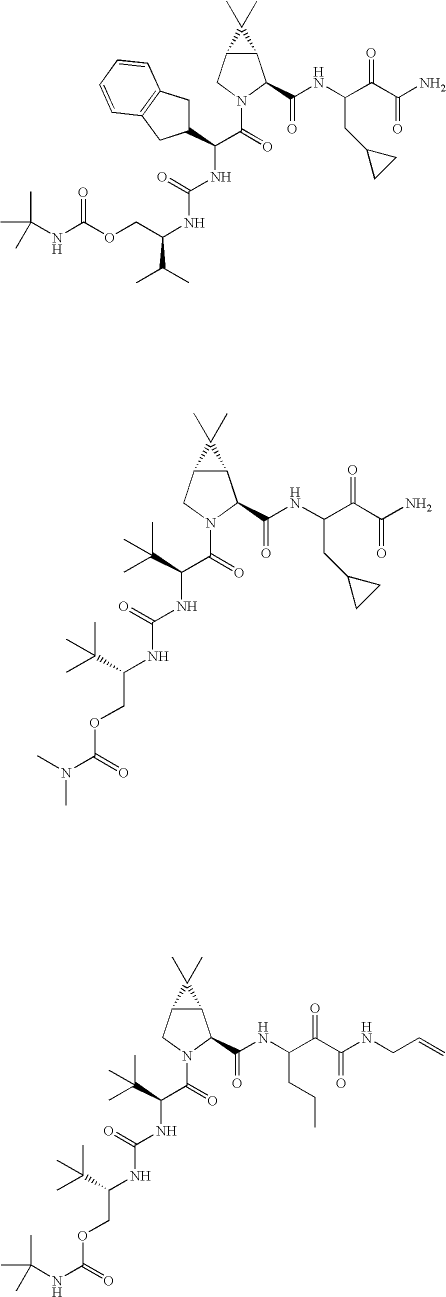 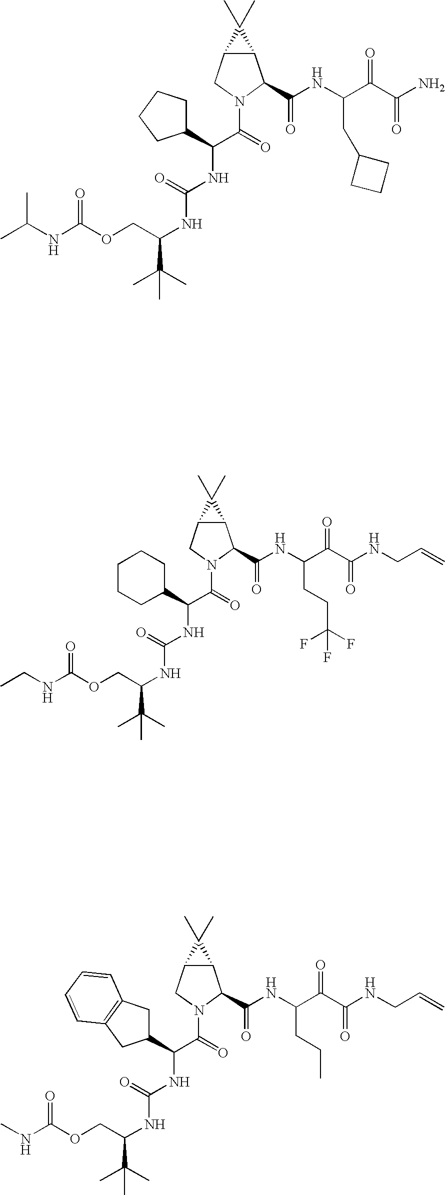 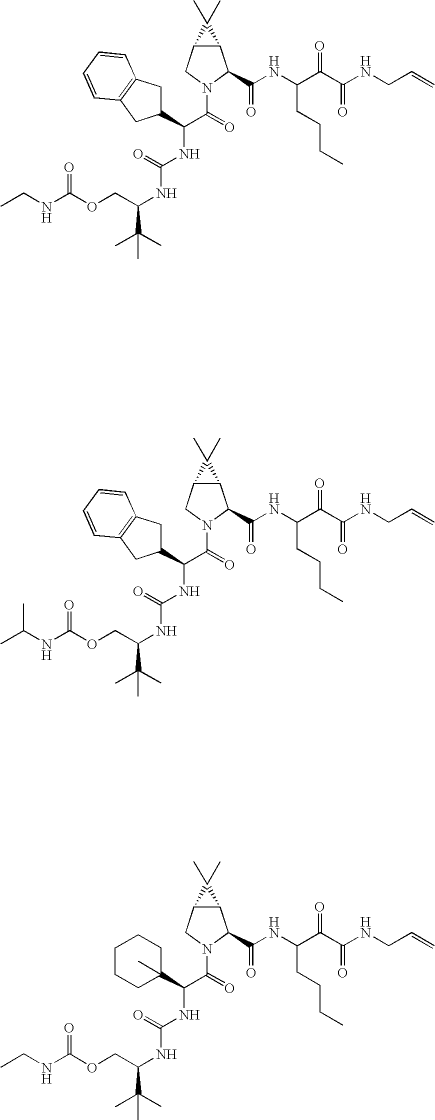 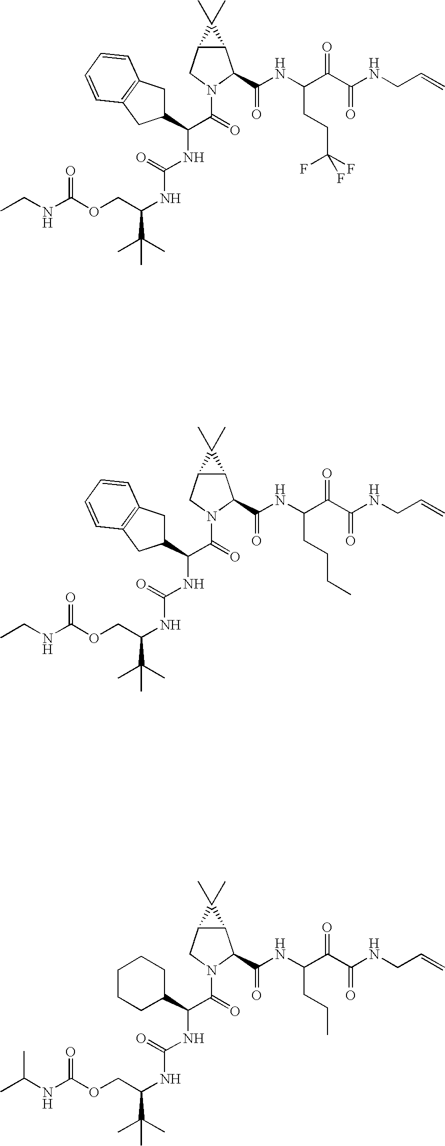 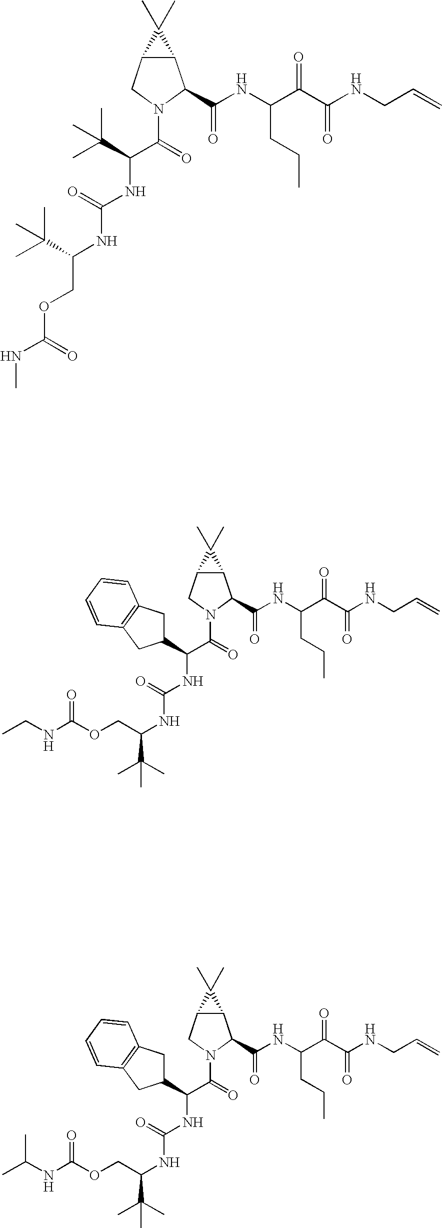 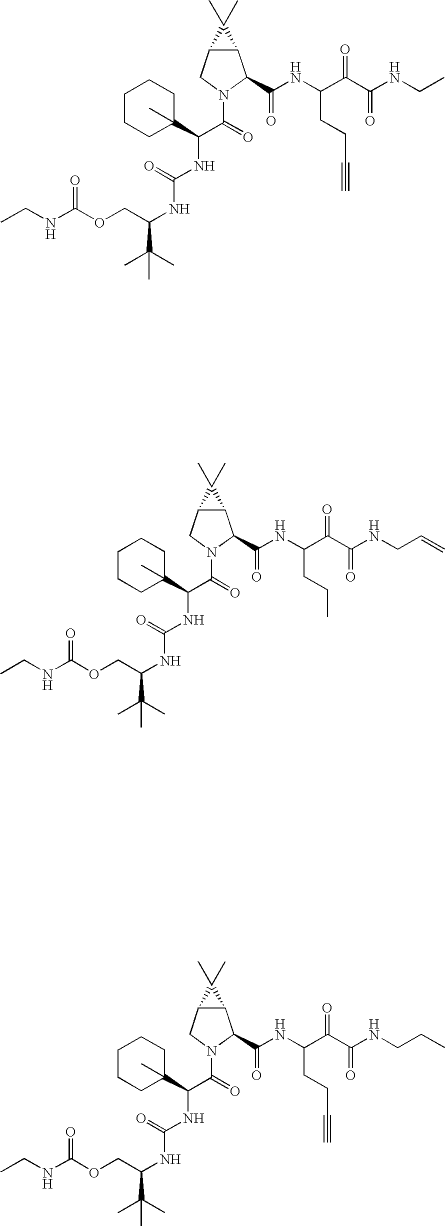 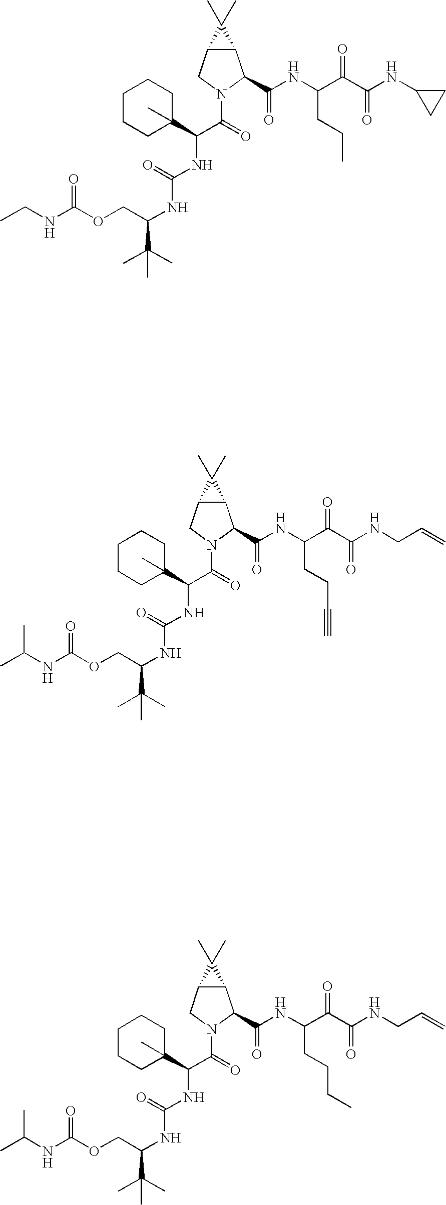 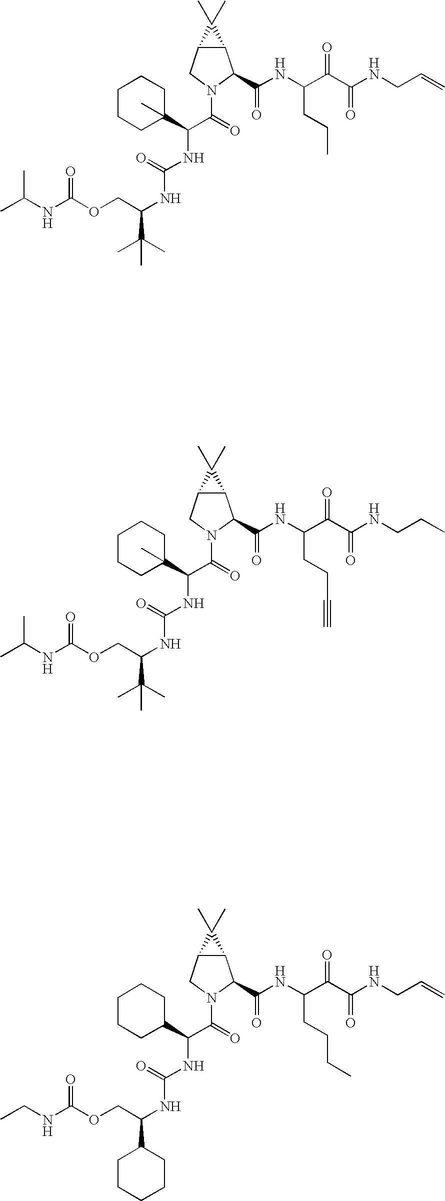 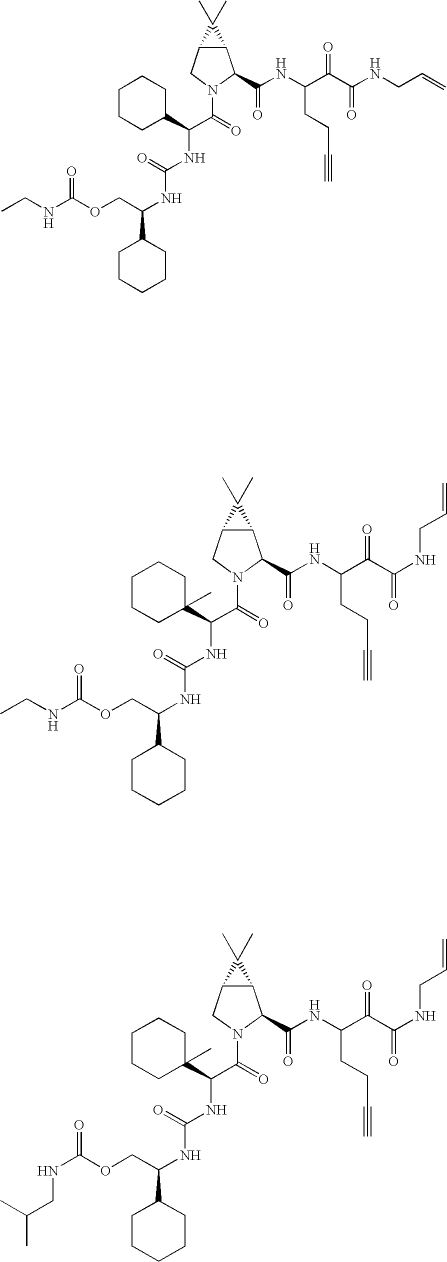 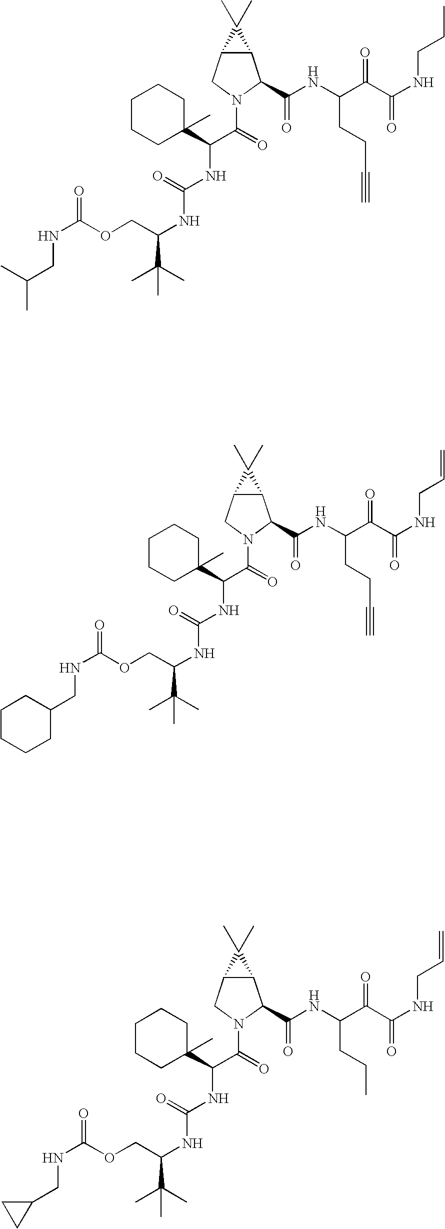 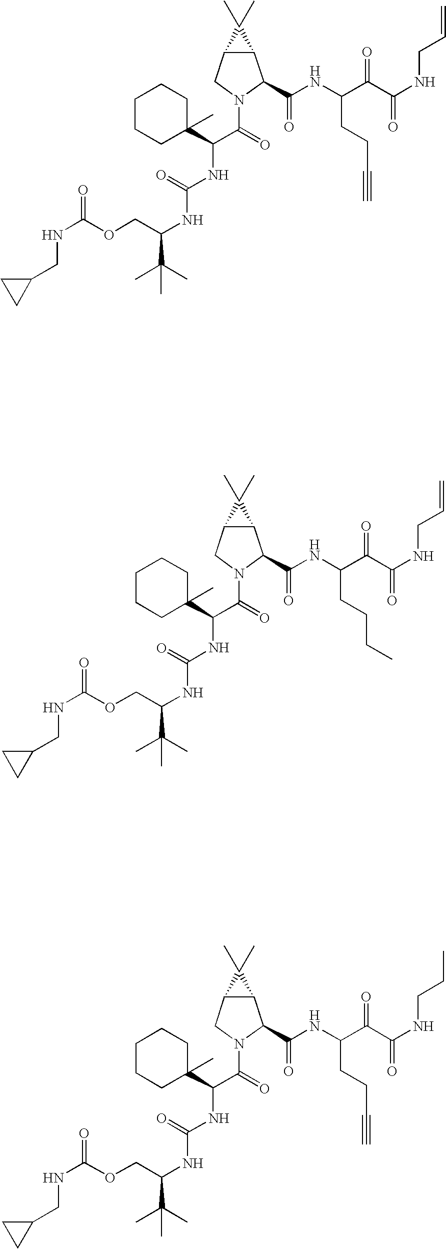 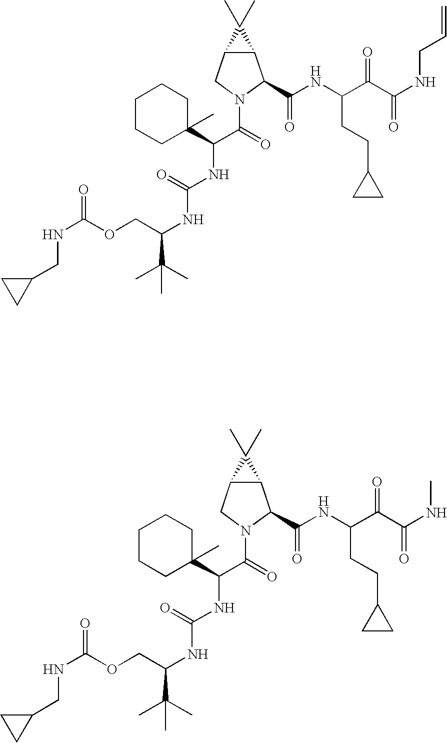
or a pharmaceutically acceptable salt, solvate or ester thereof.

Compounds of formula XIII are disclosed in U.S. patent application Ser. No. 11/065,647 filed Feb. 24, 2005. The preparation of these compounds is disclosed in the experimental section of this application set forth hereinbelow.

Non-limiting examples of certain compounds disclosed in U.S. patent application Ser. No. 11/065,647 are: 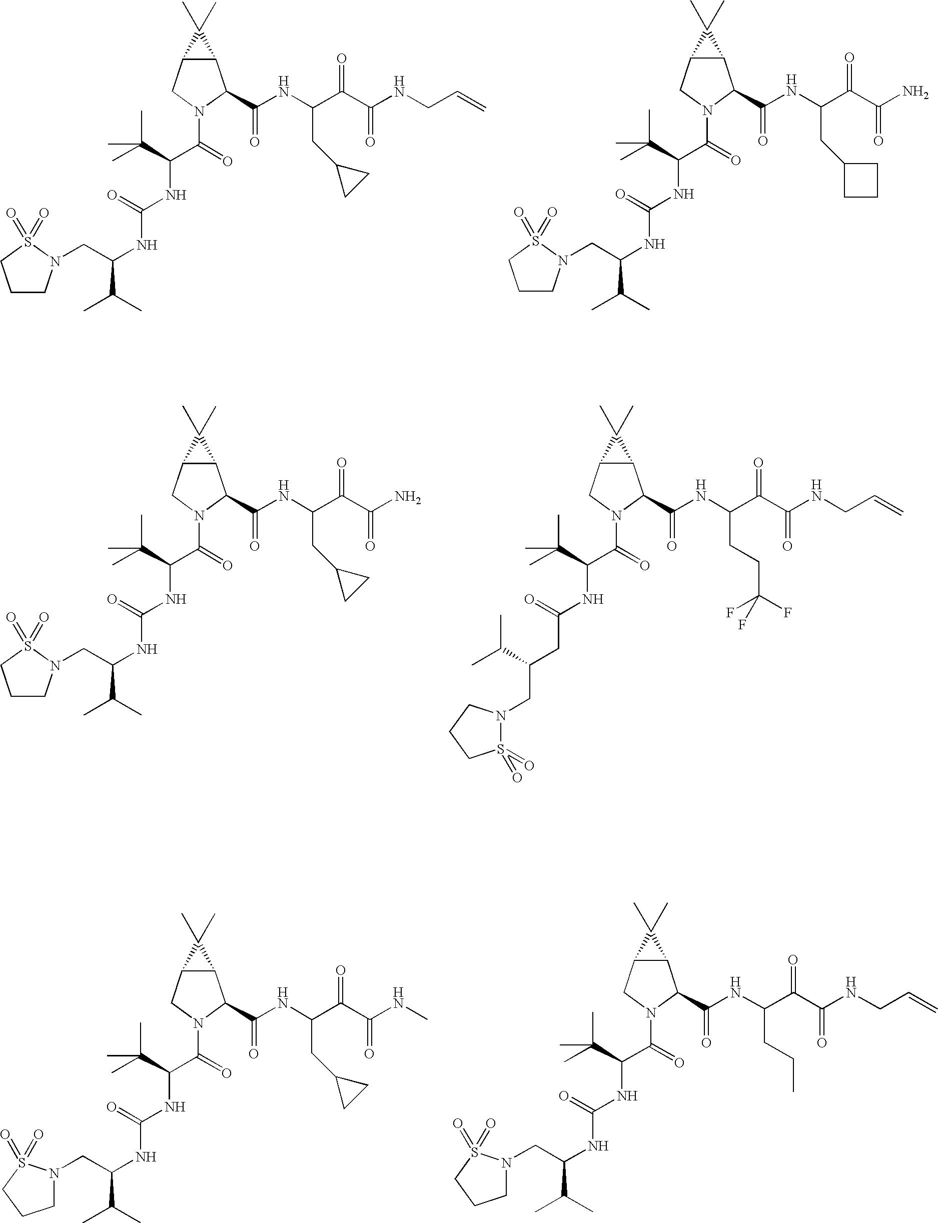 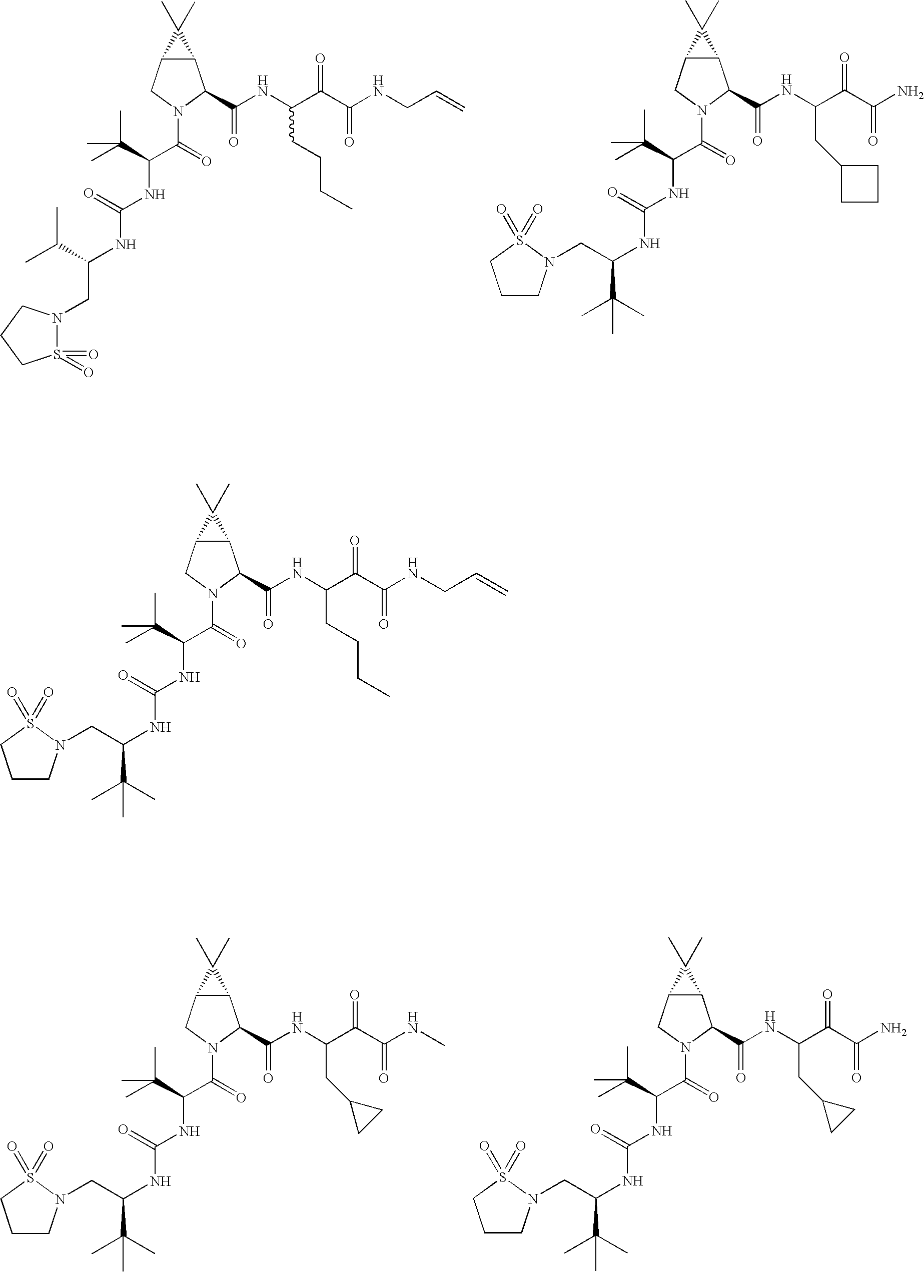 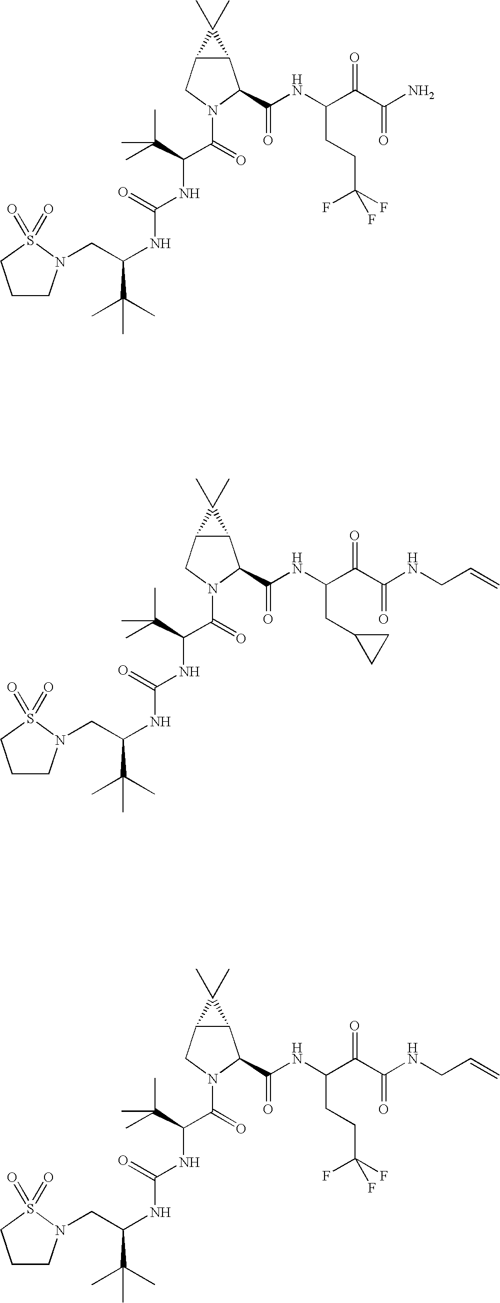 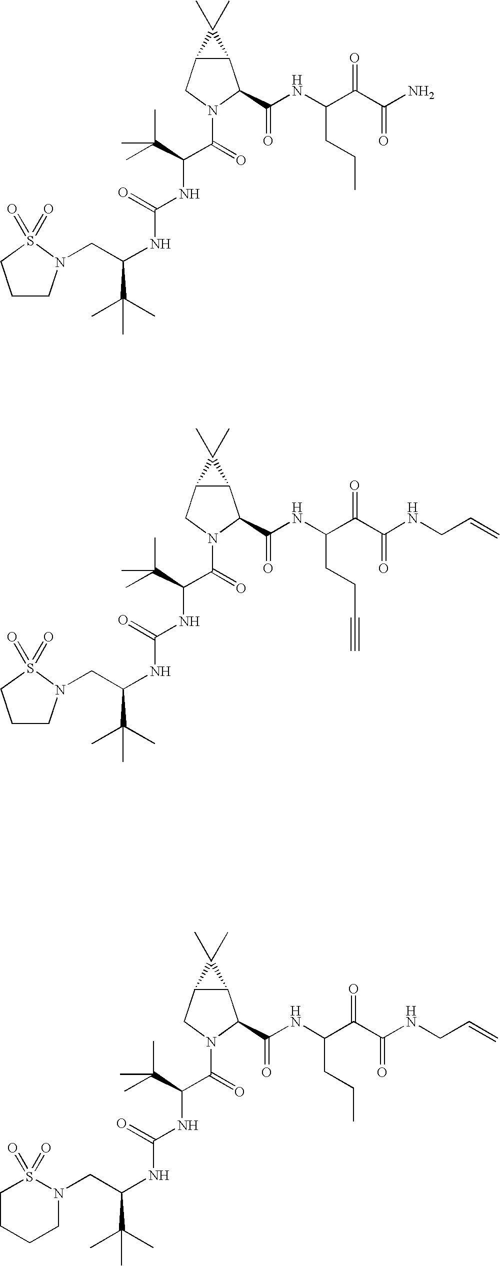 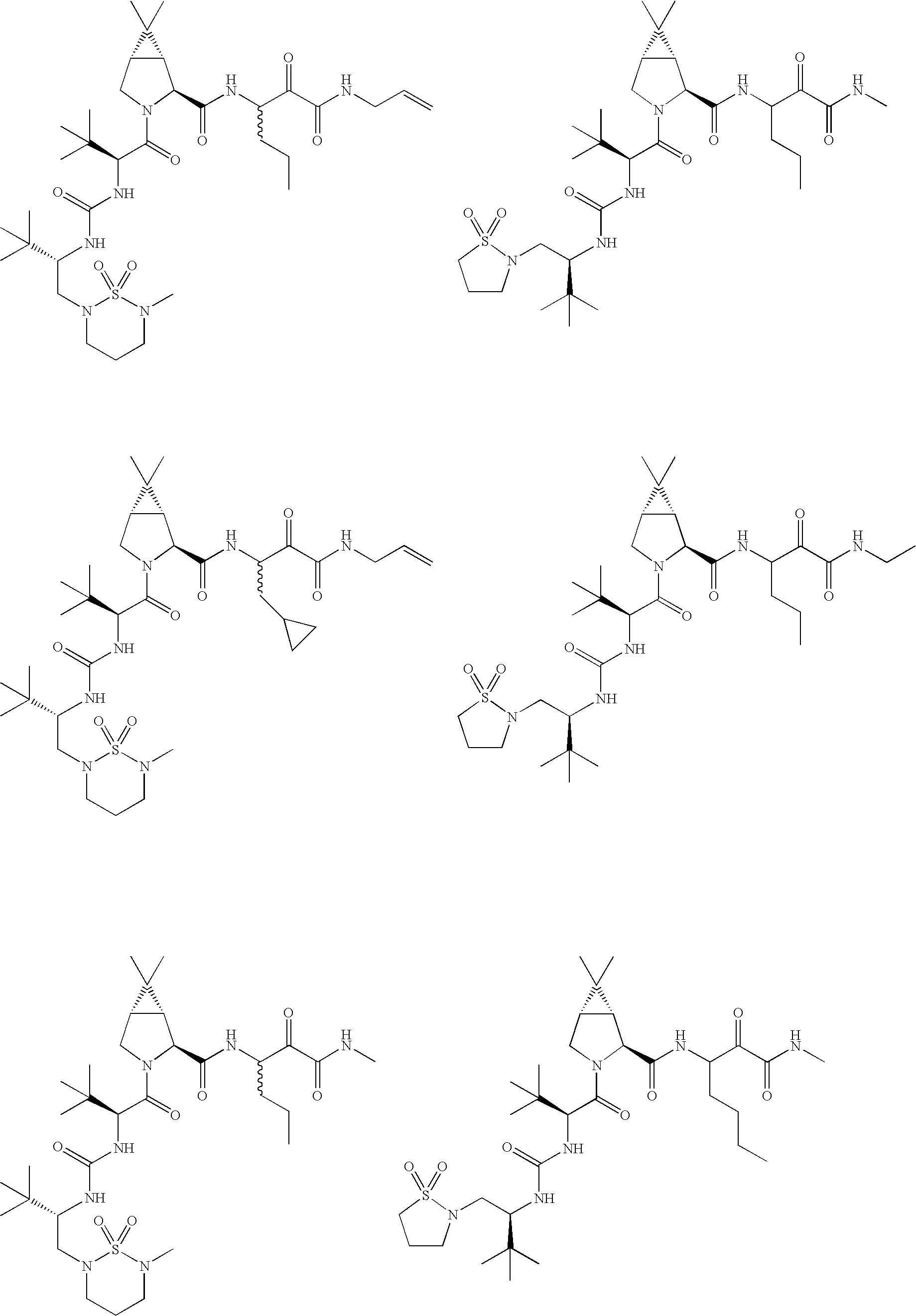 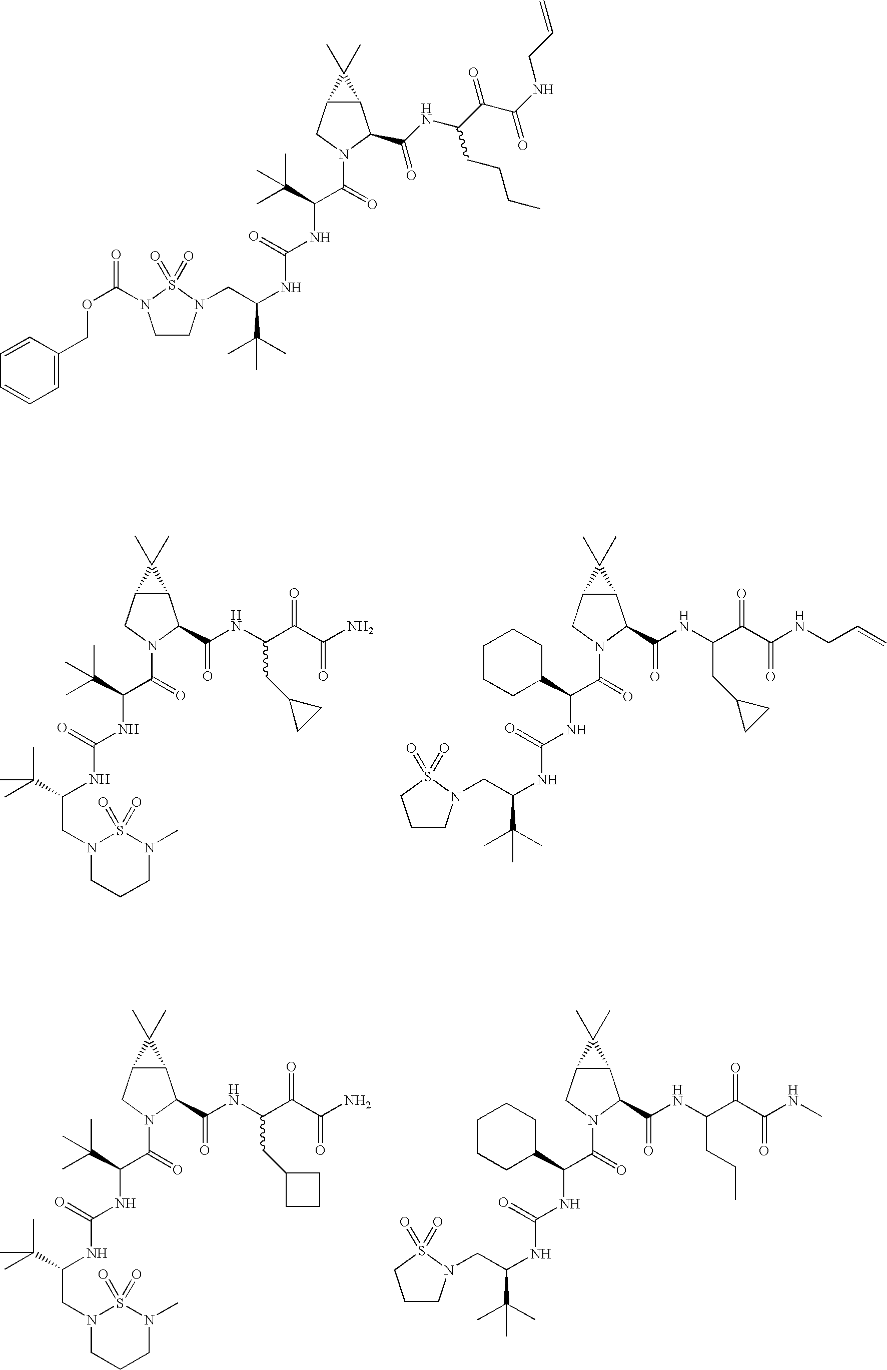 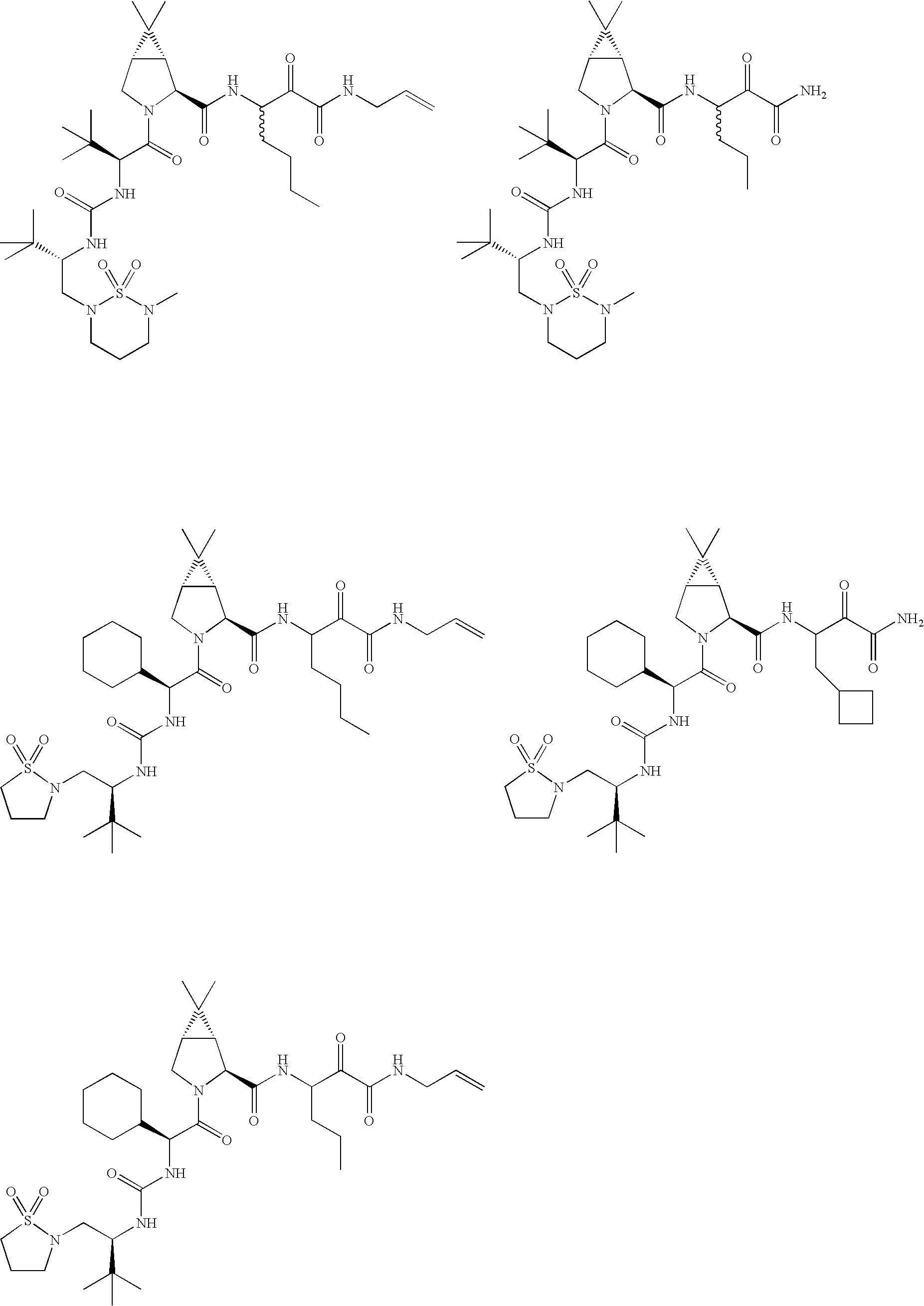 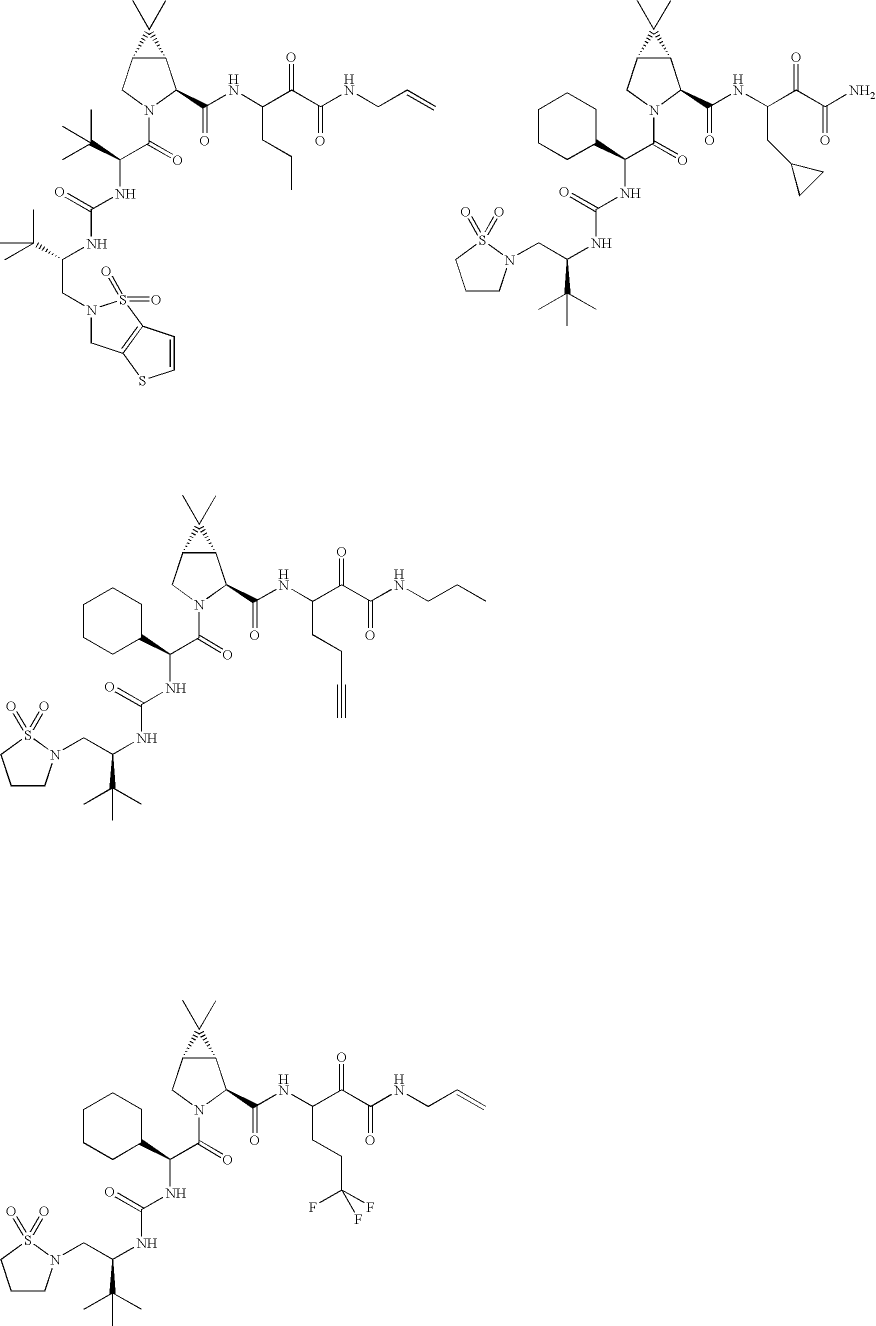 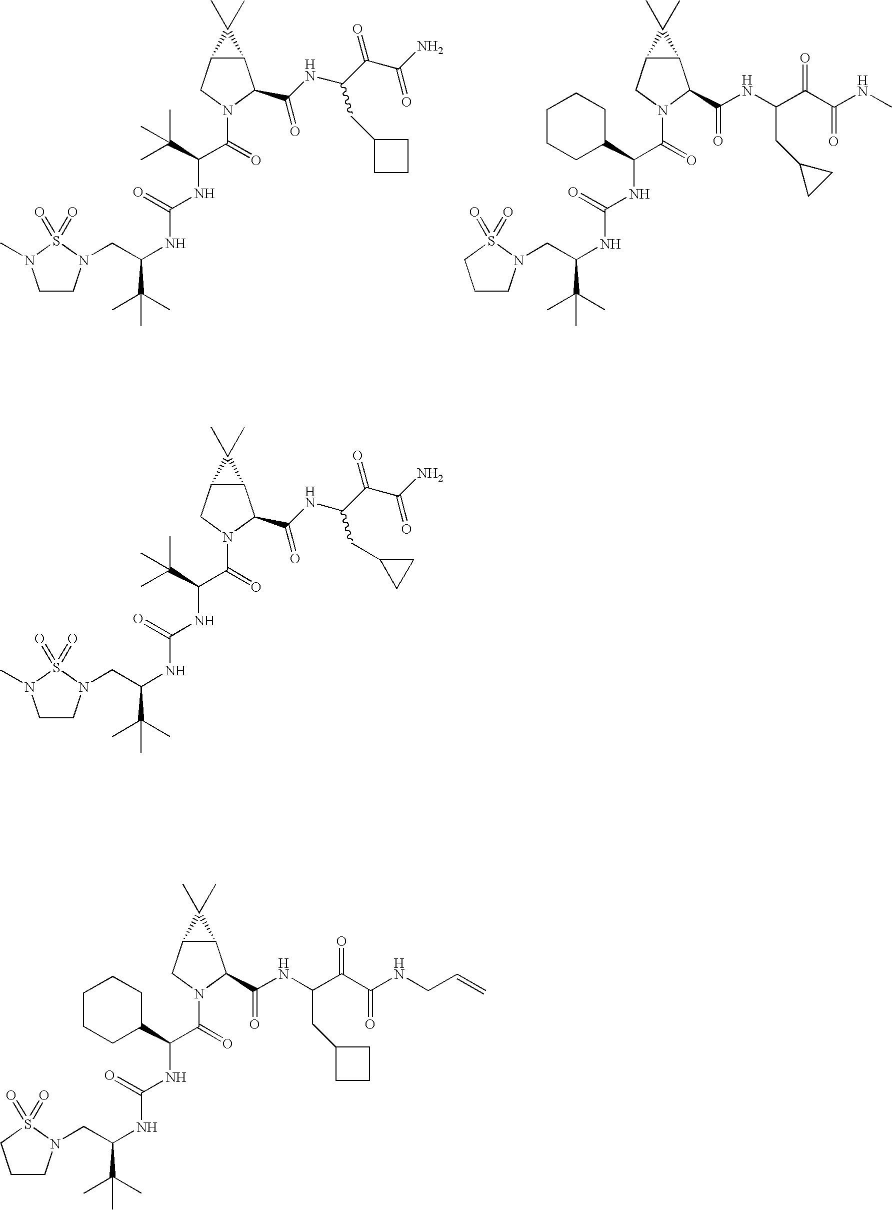 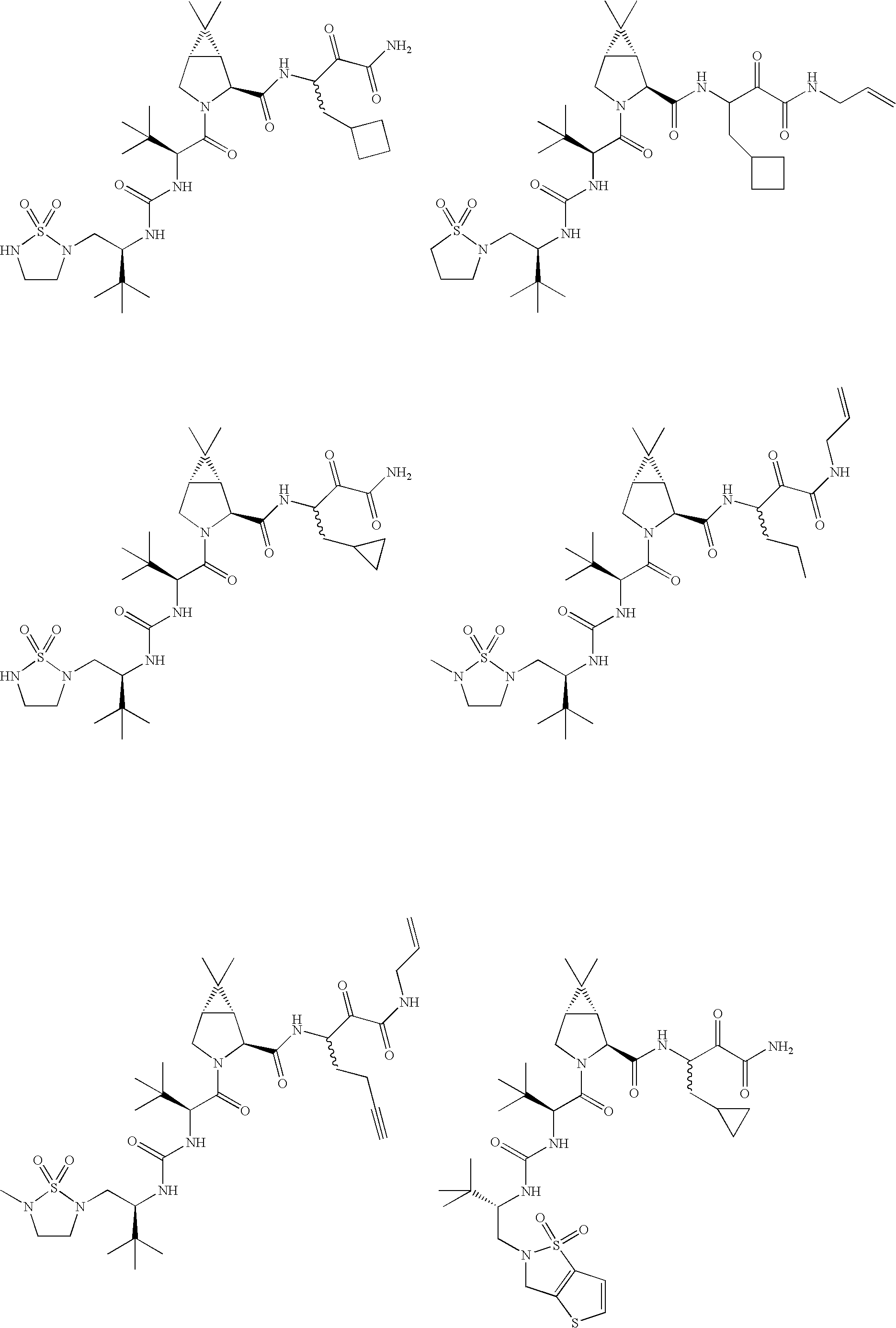 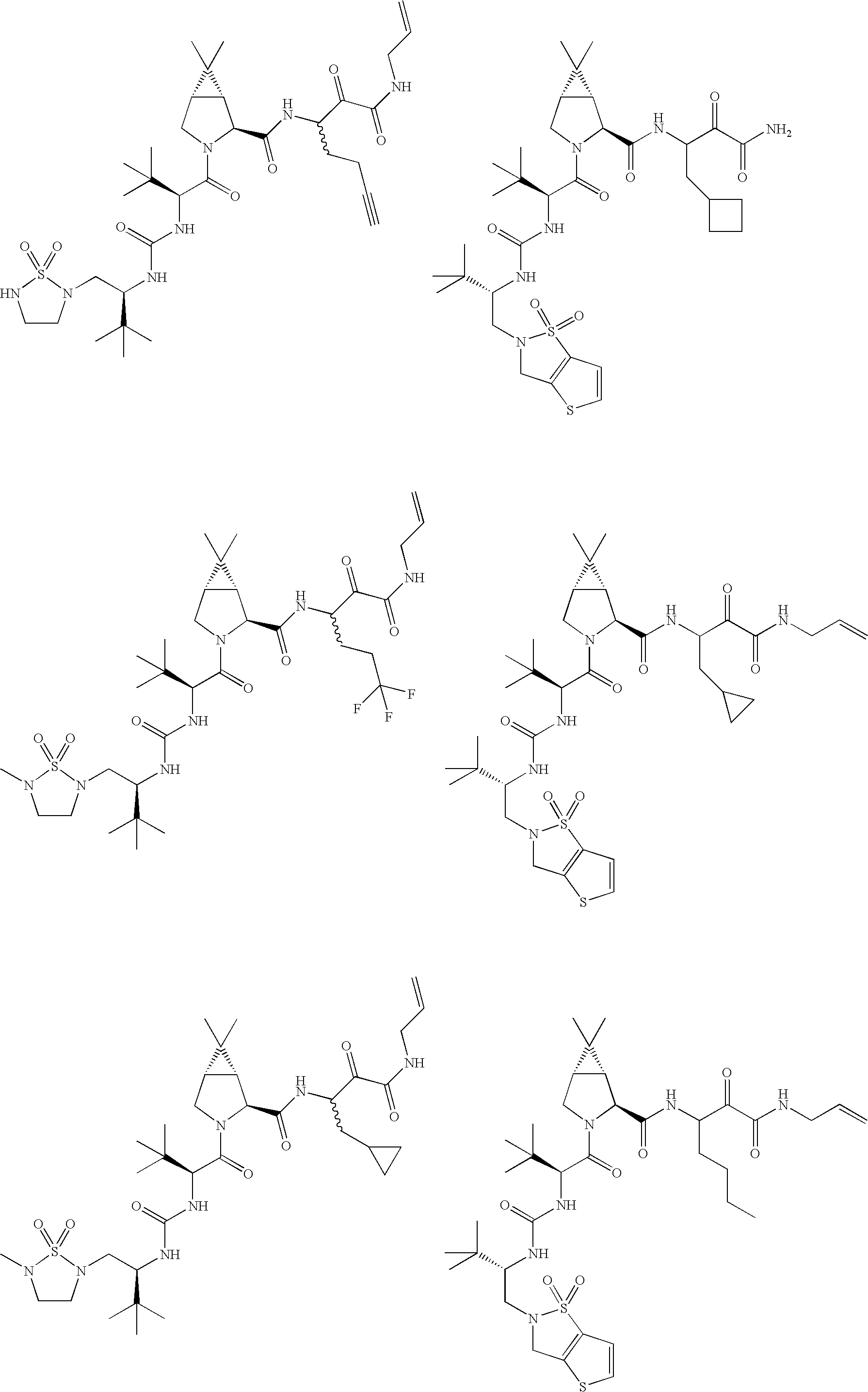 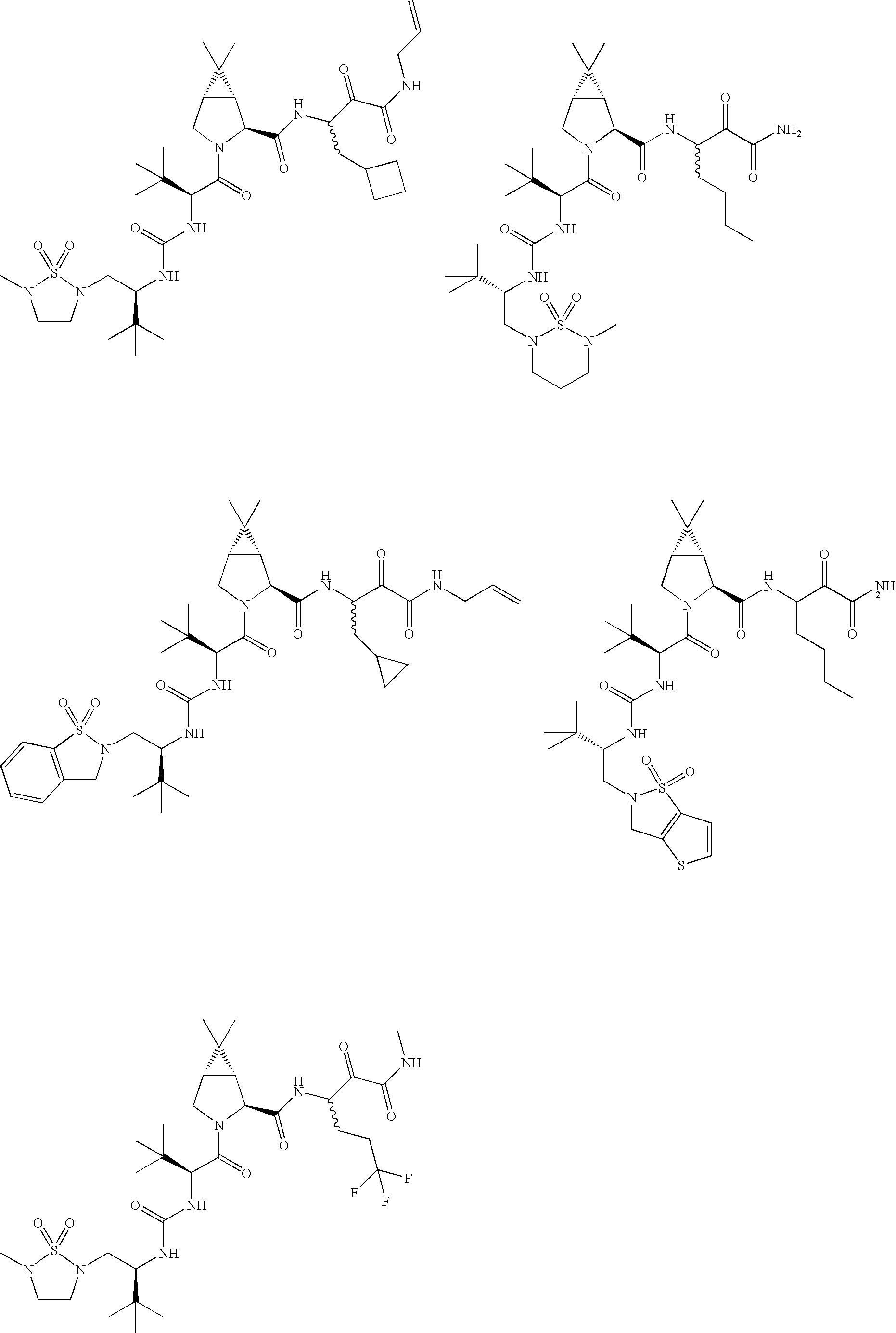 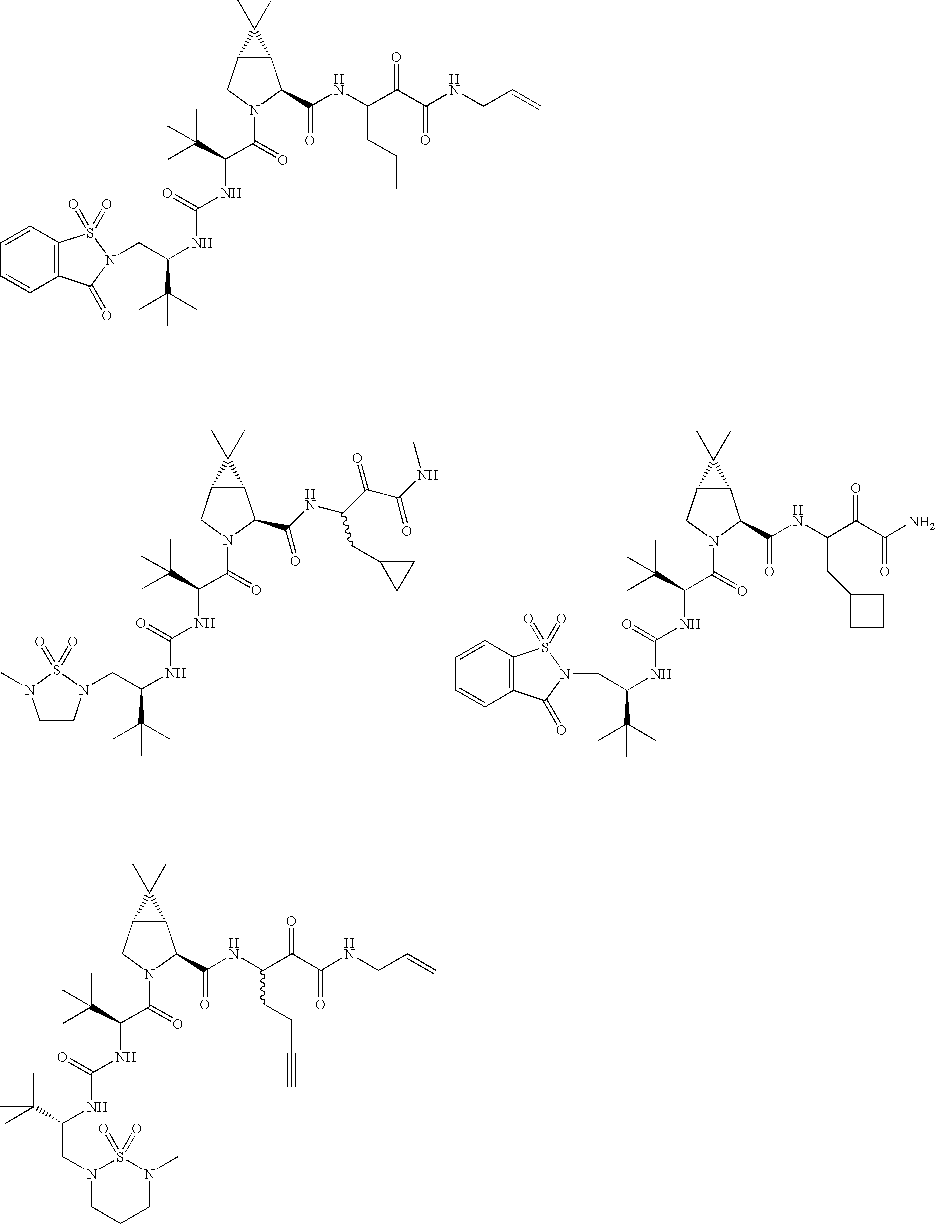 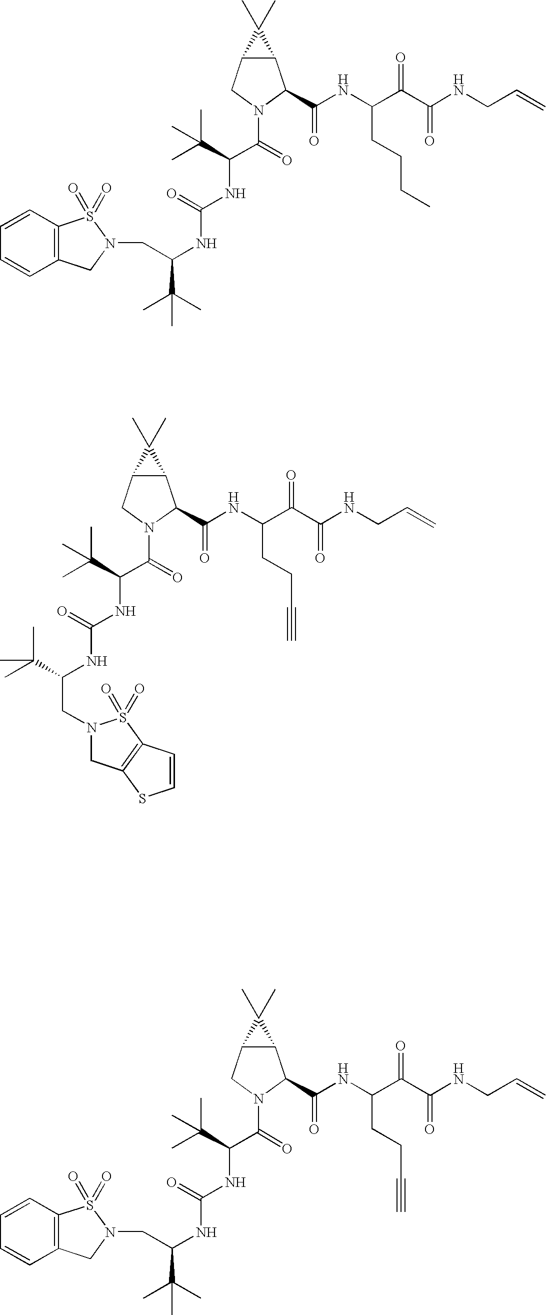 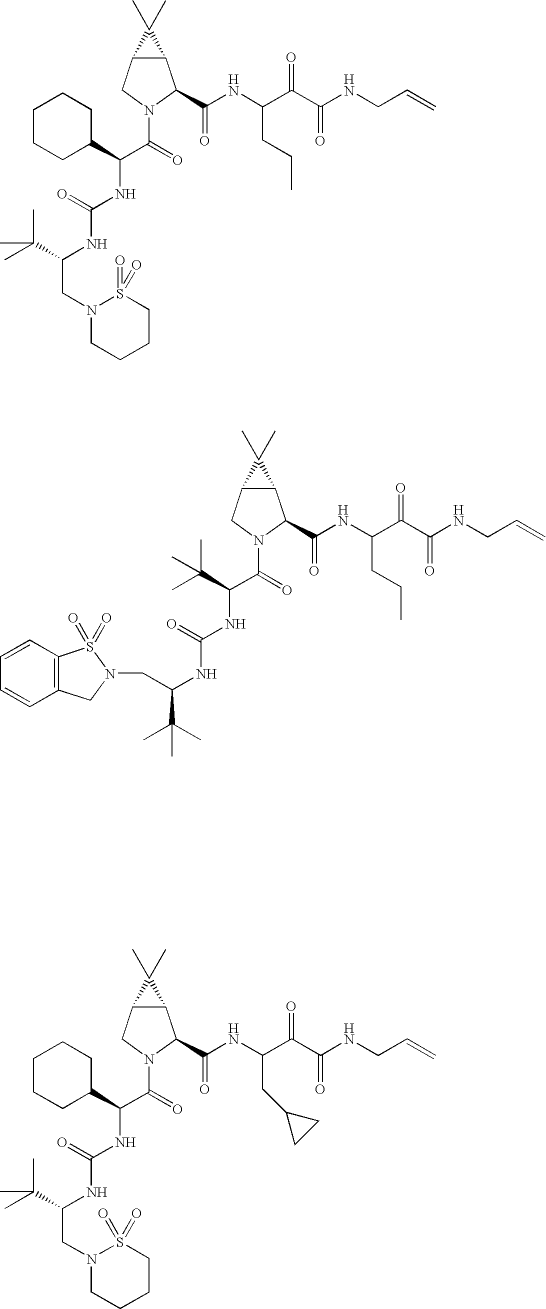 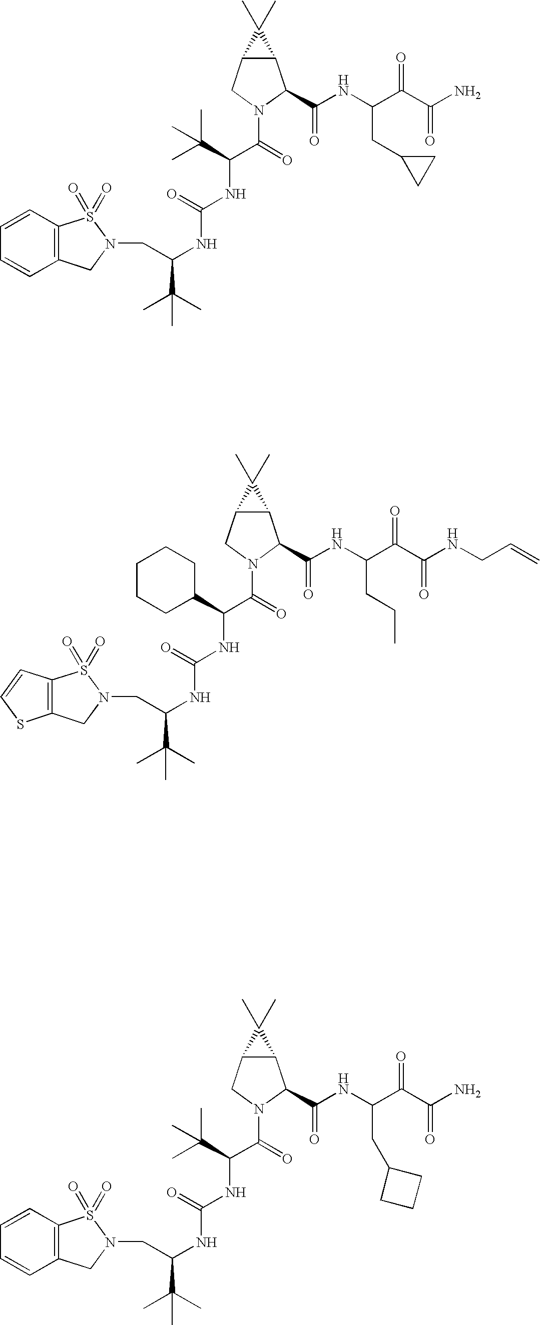 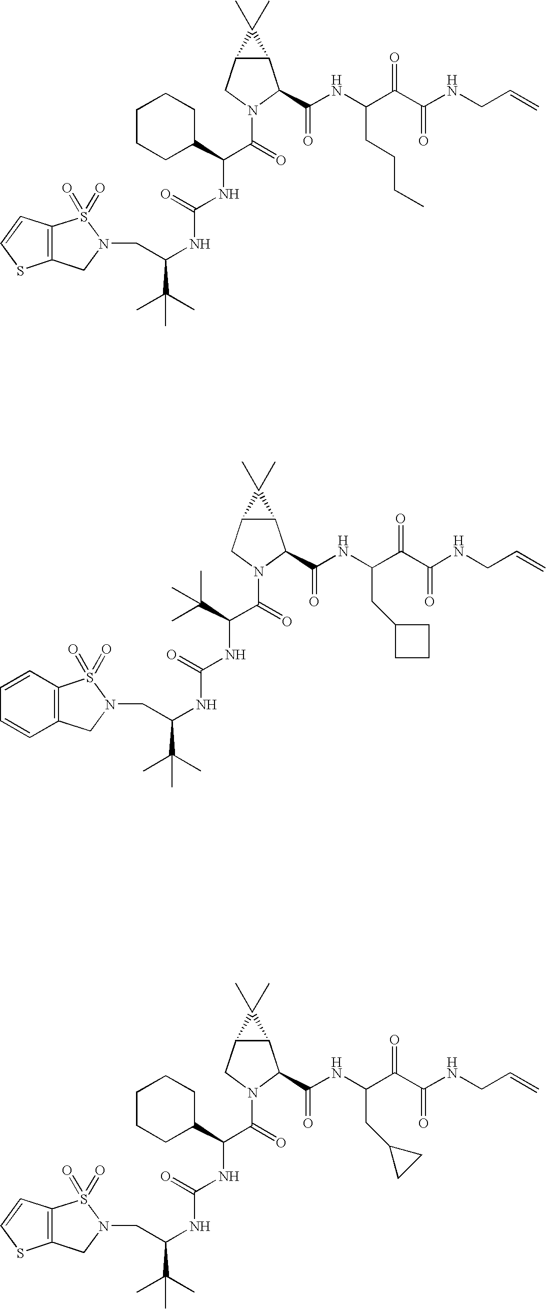 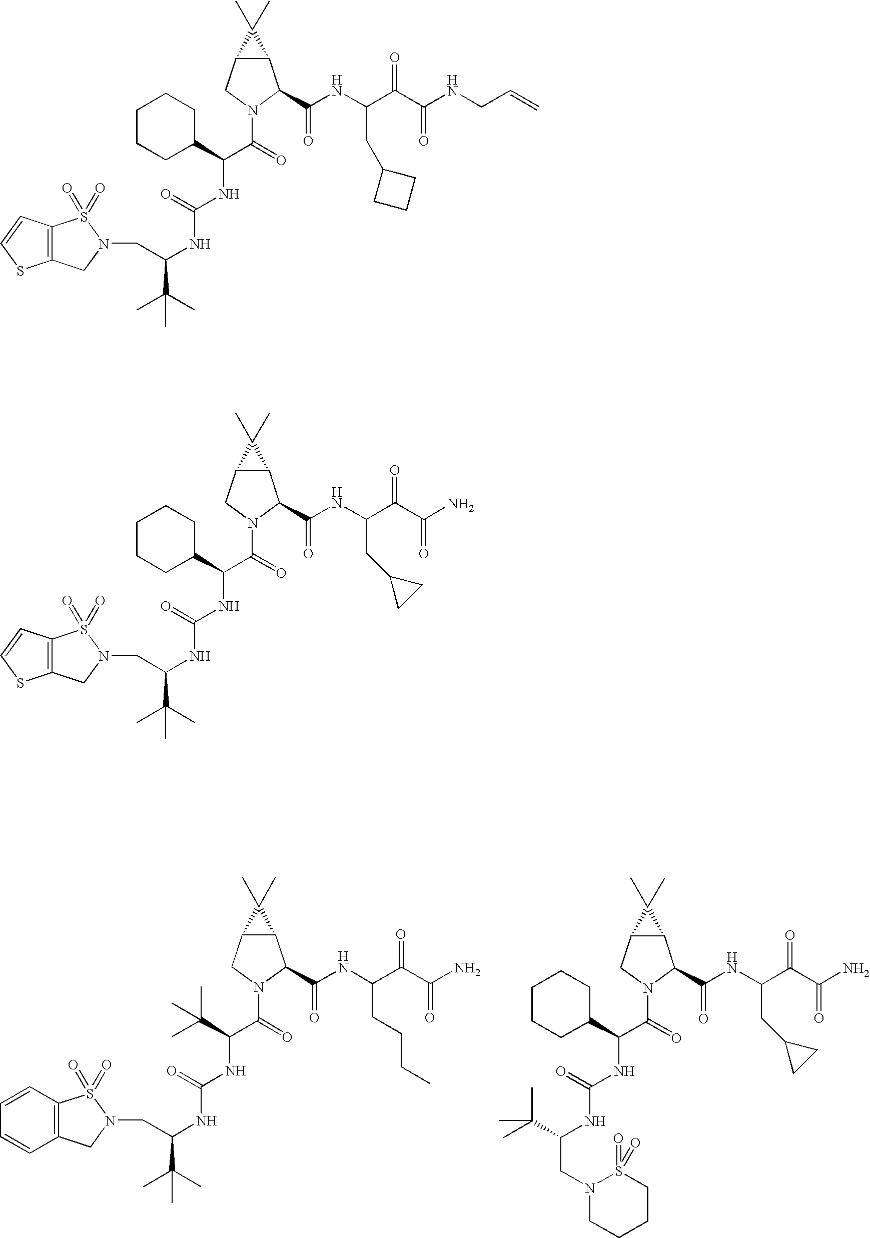 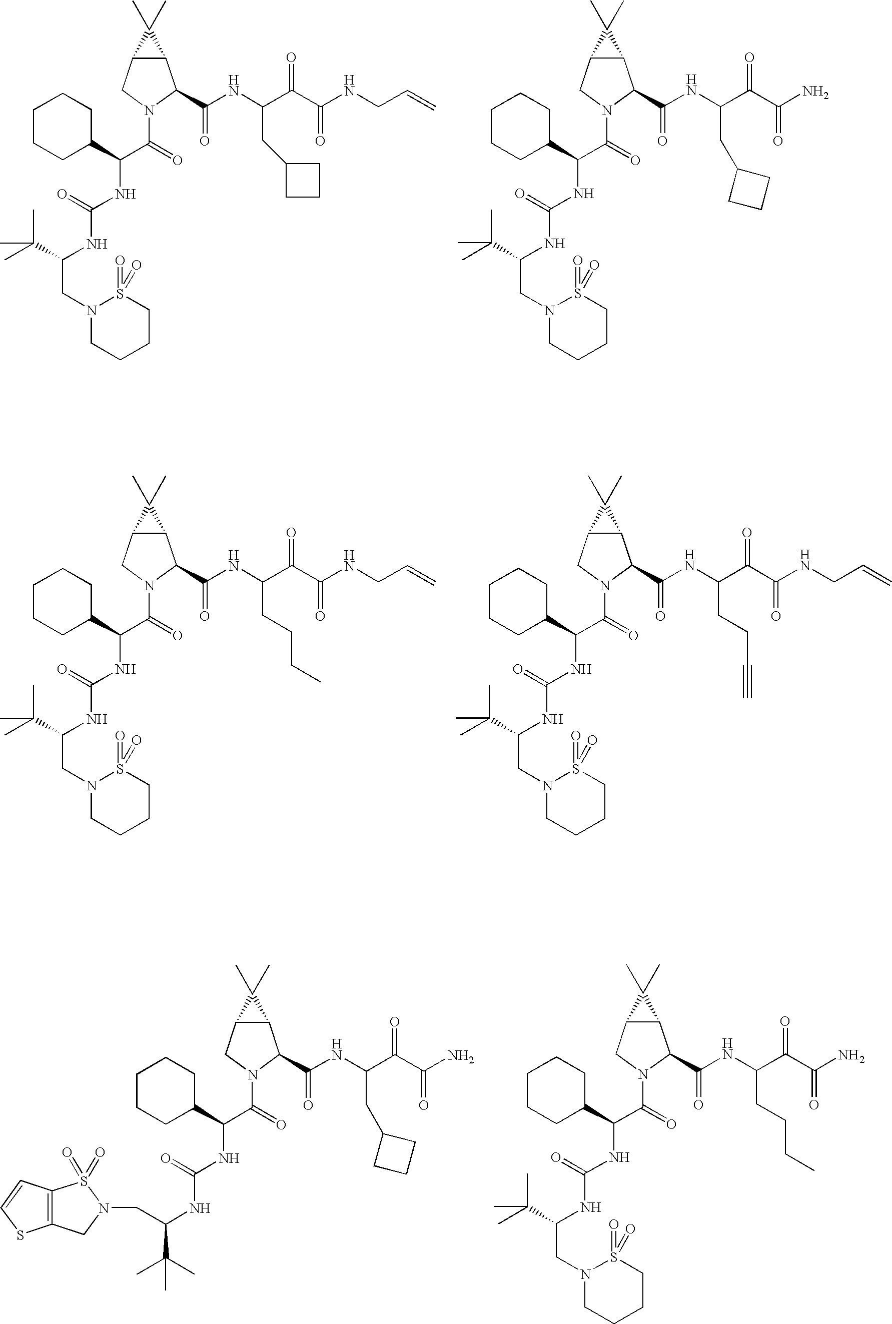 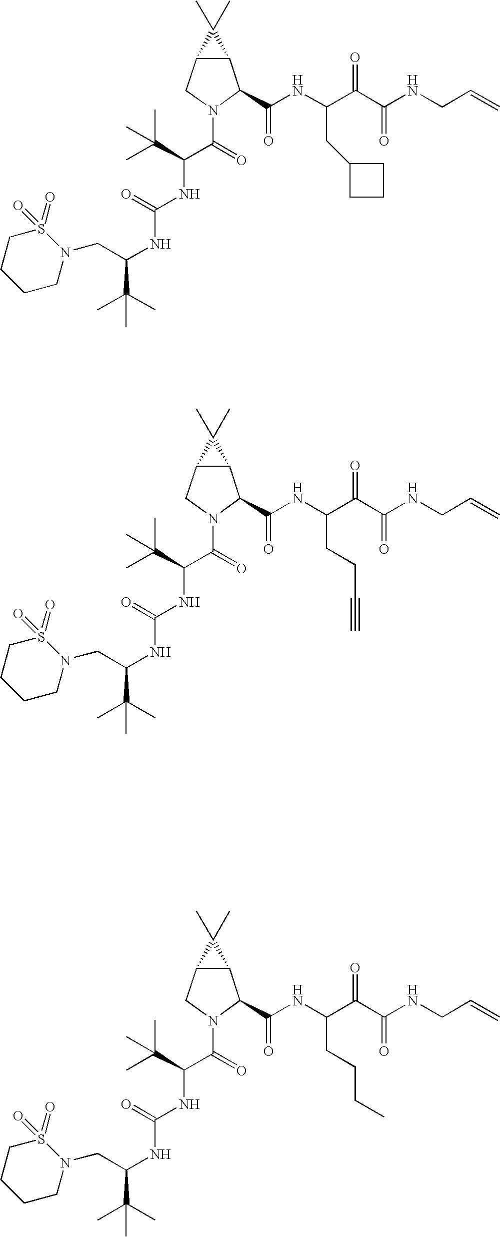 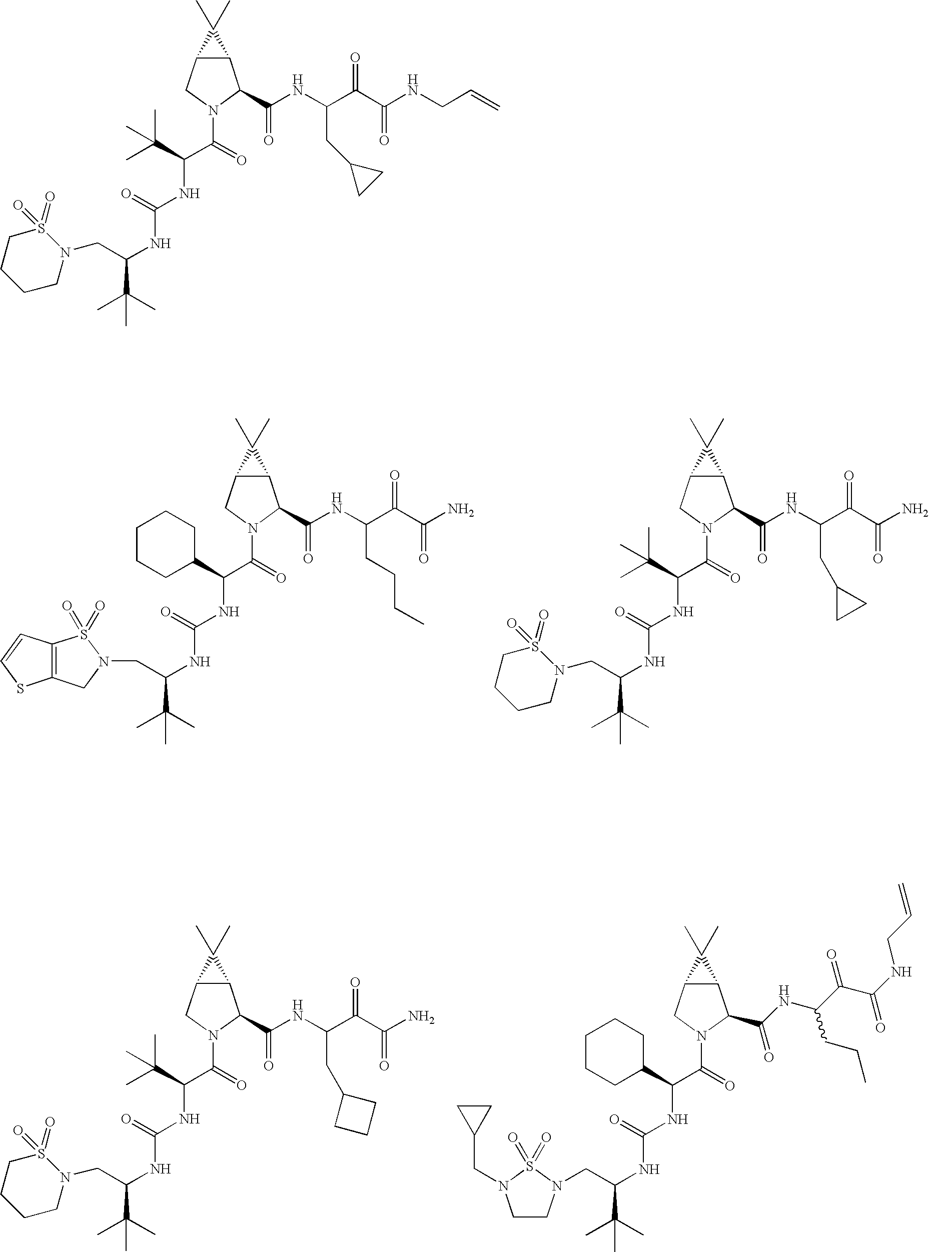 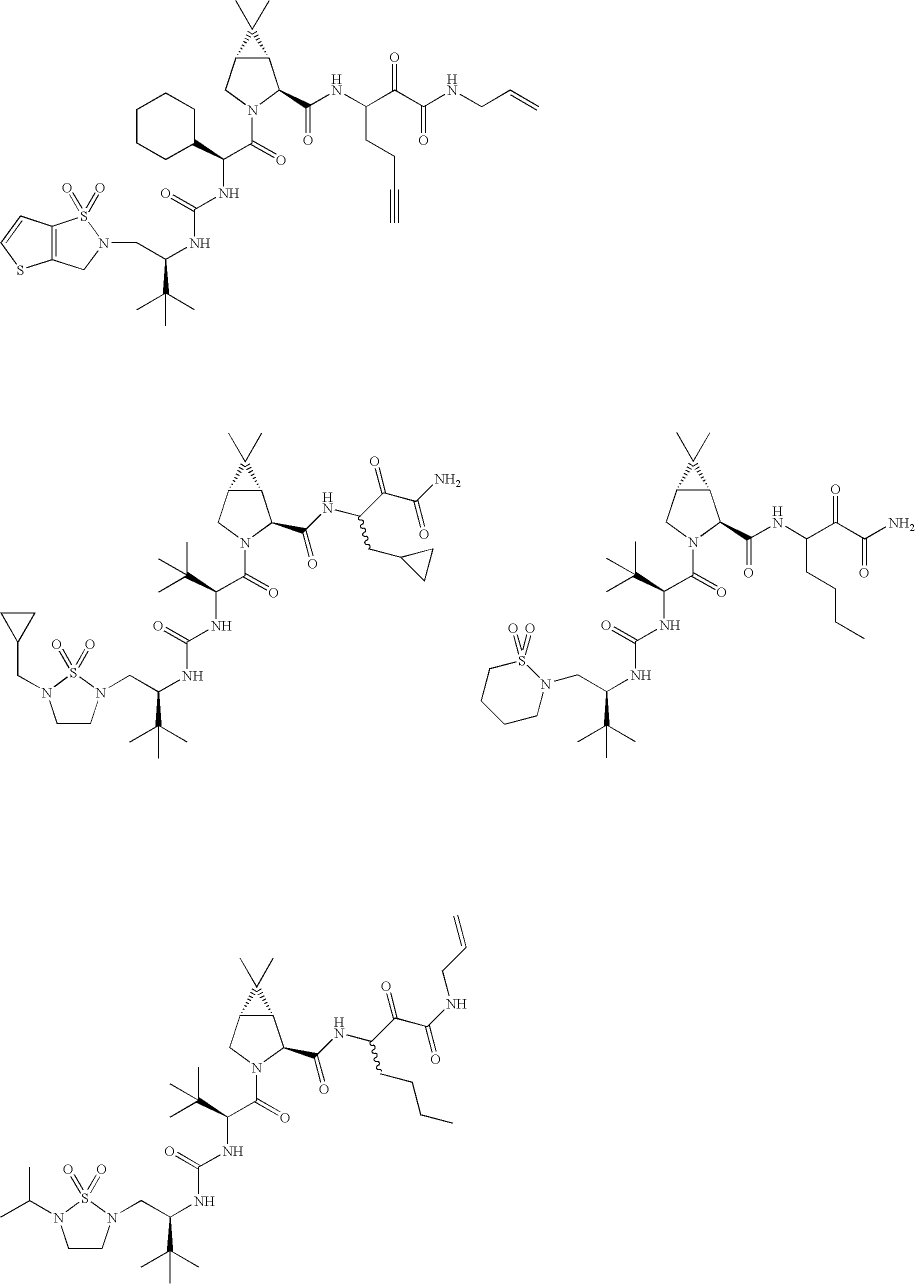 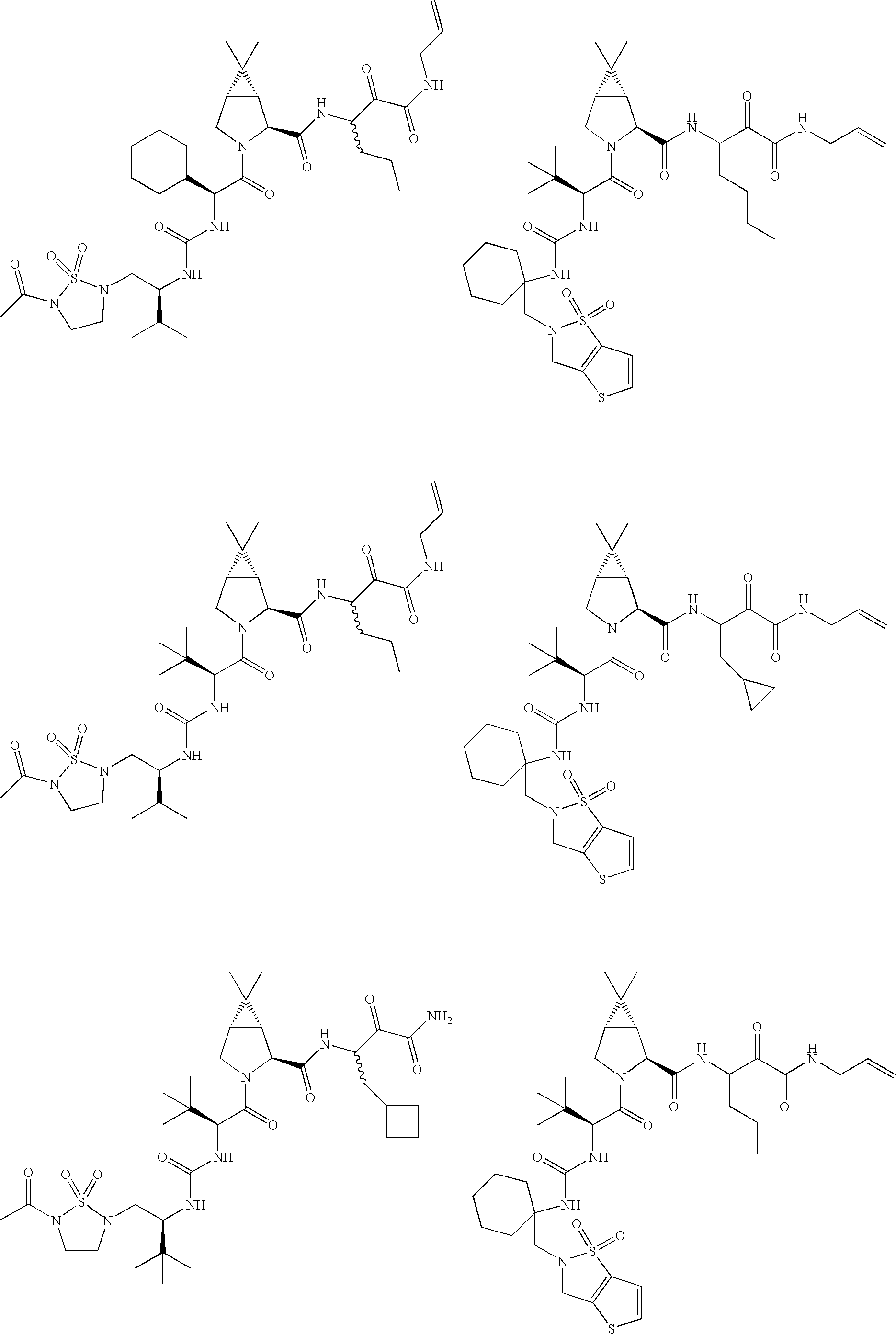 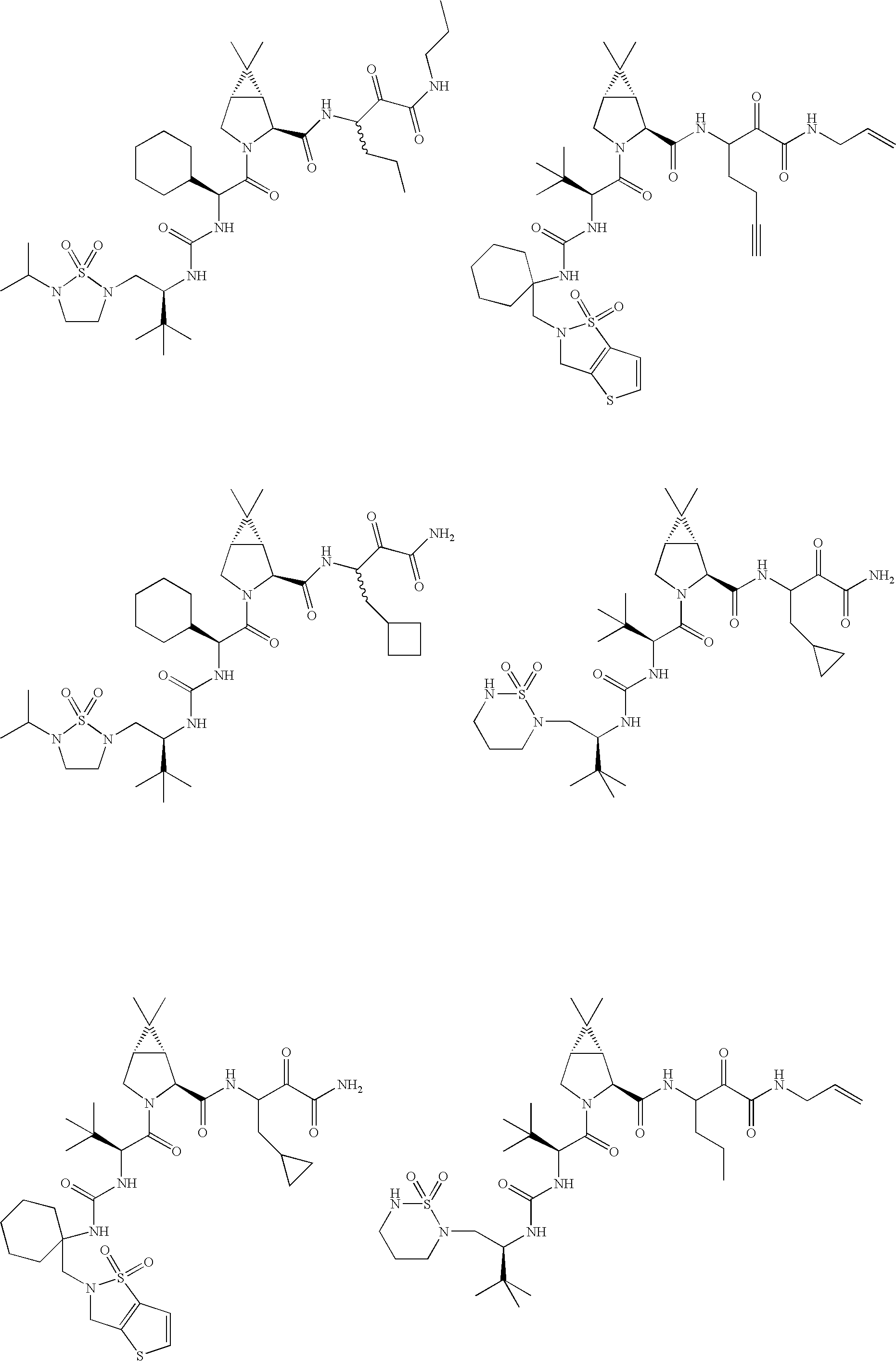 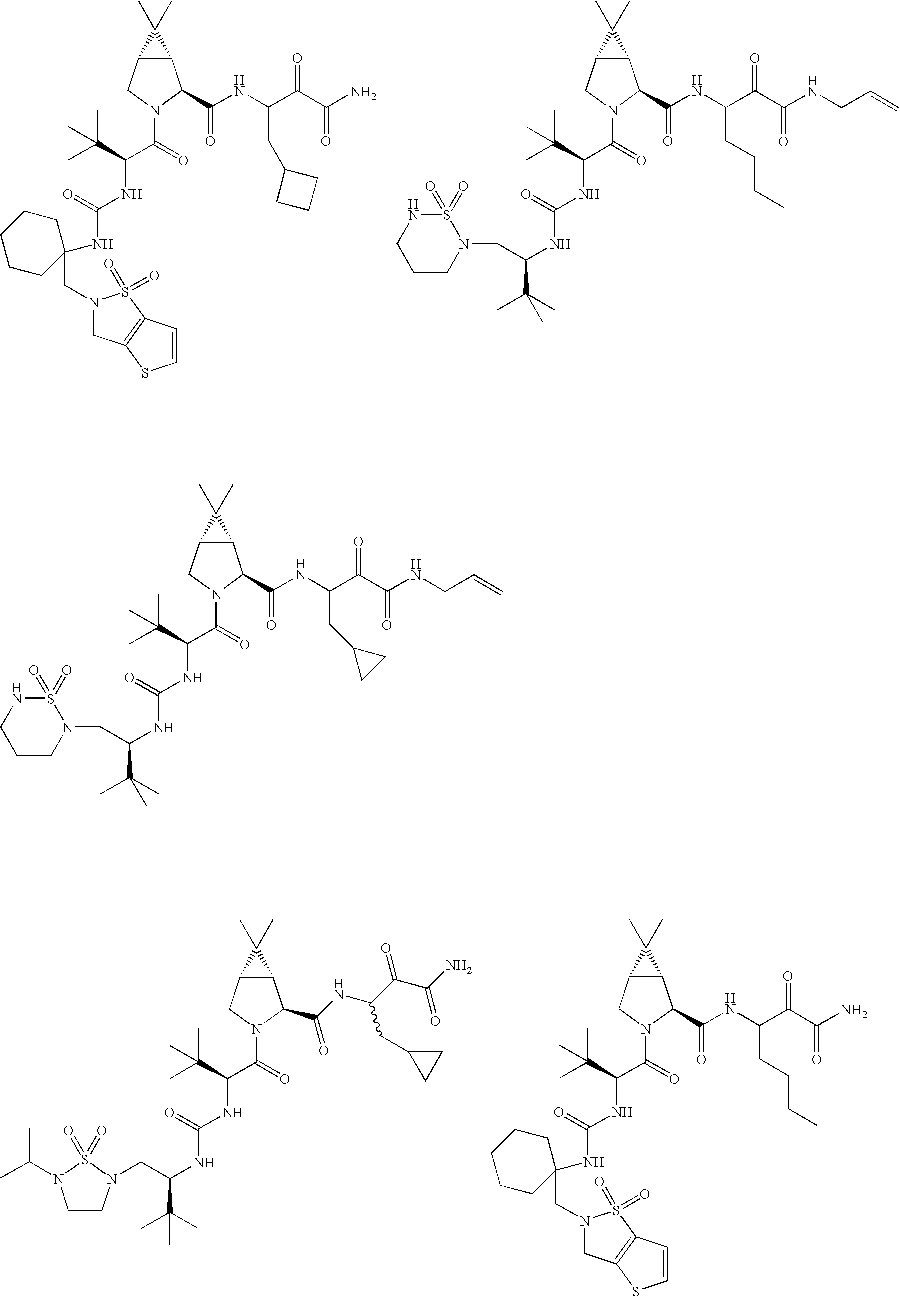 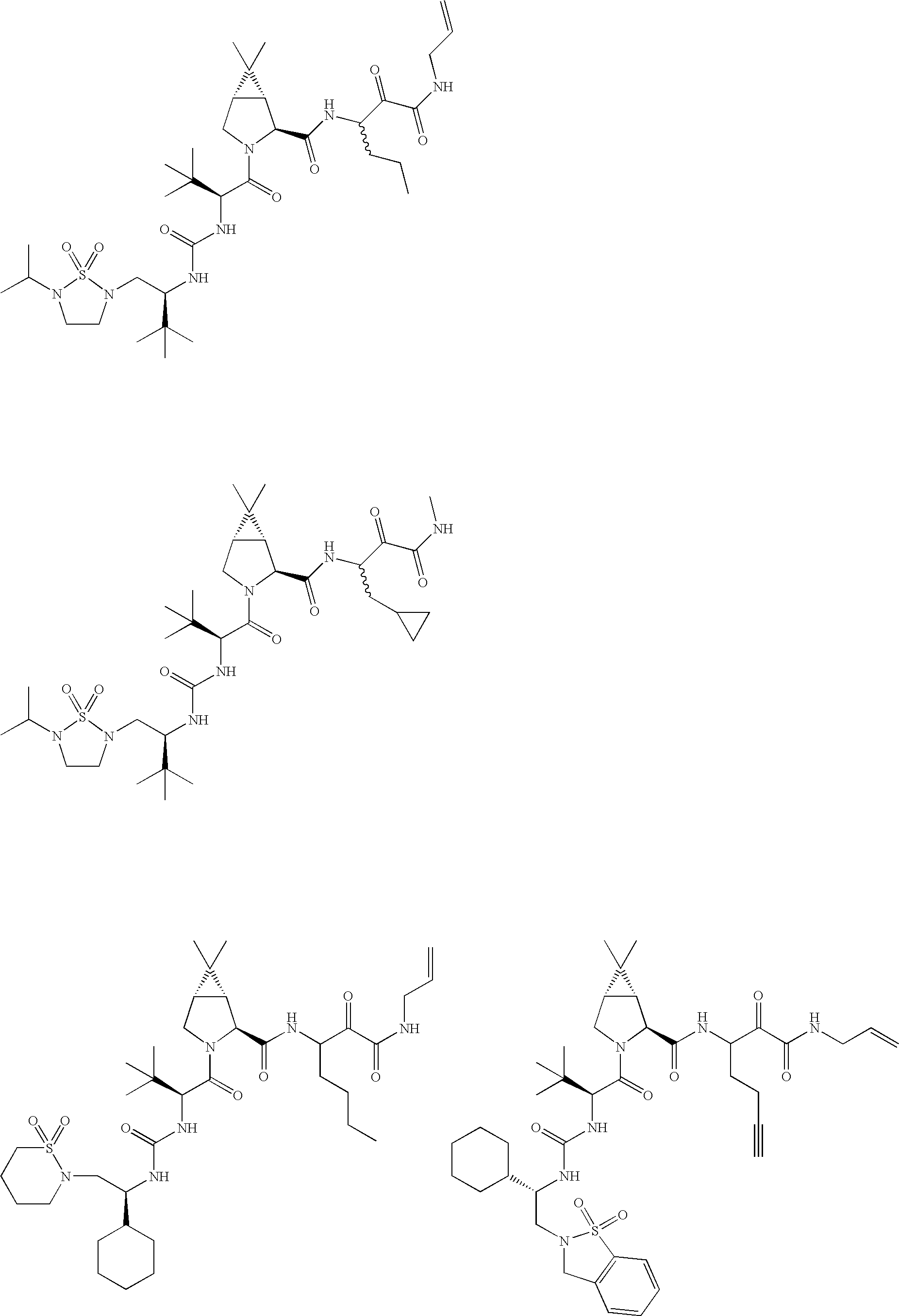 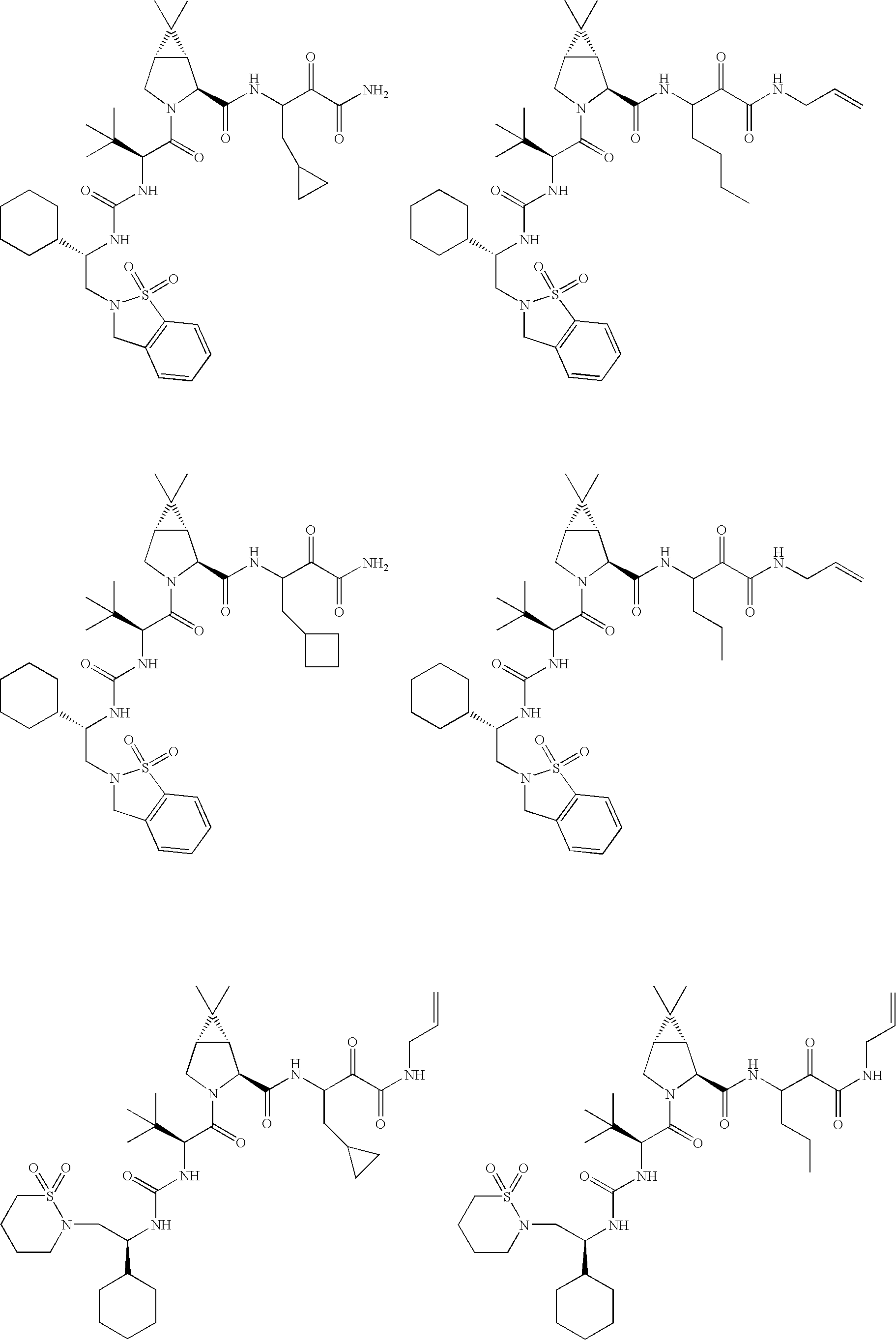 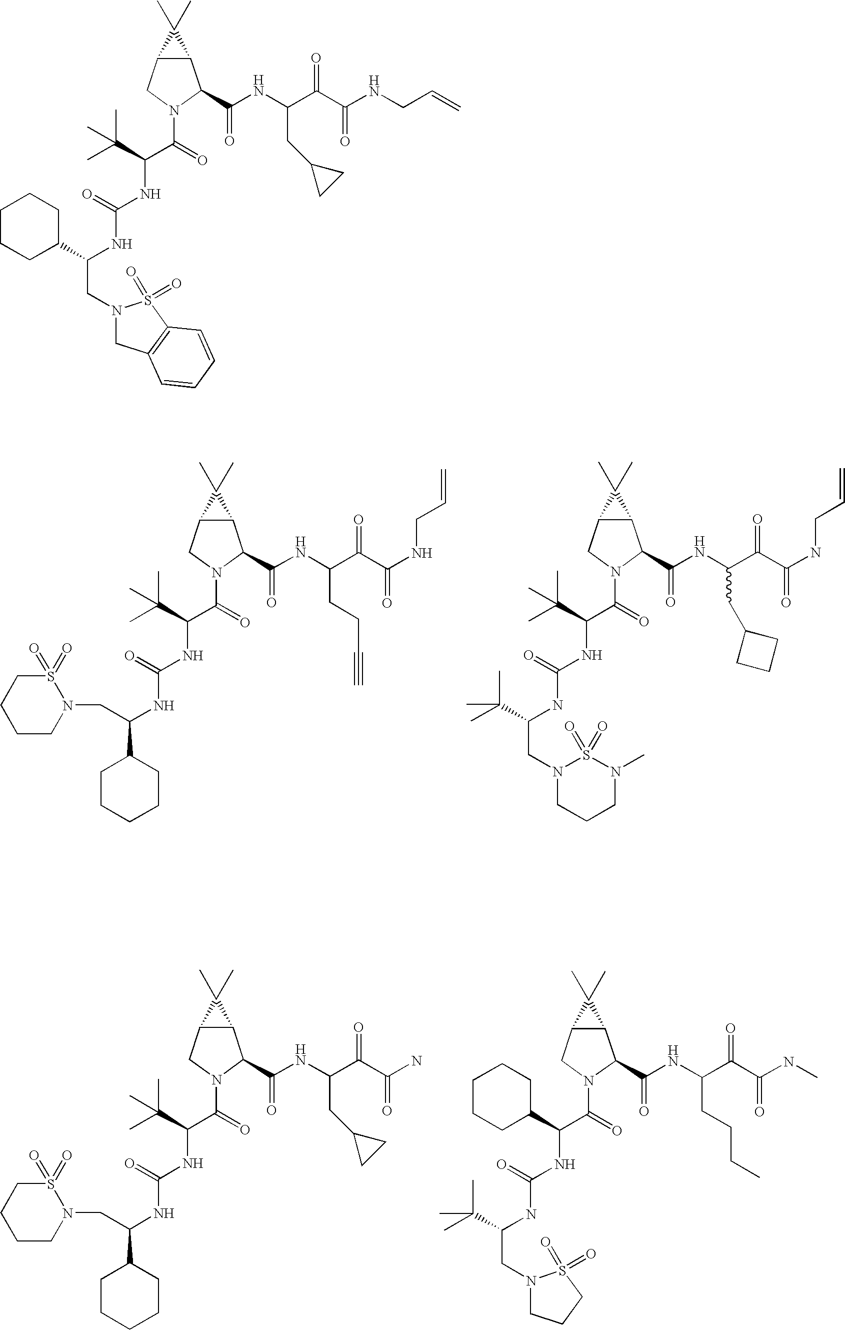 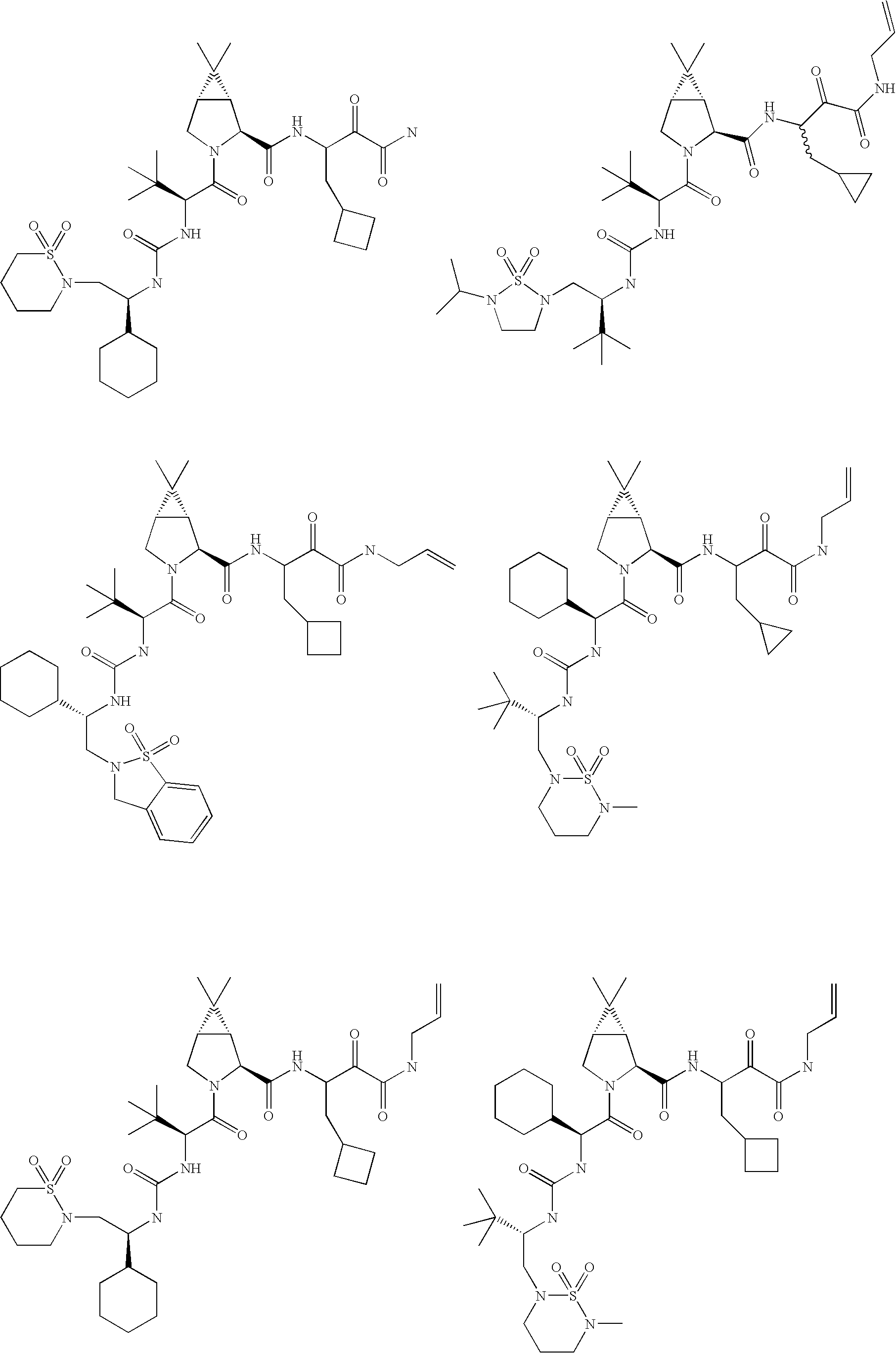 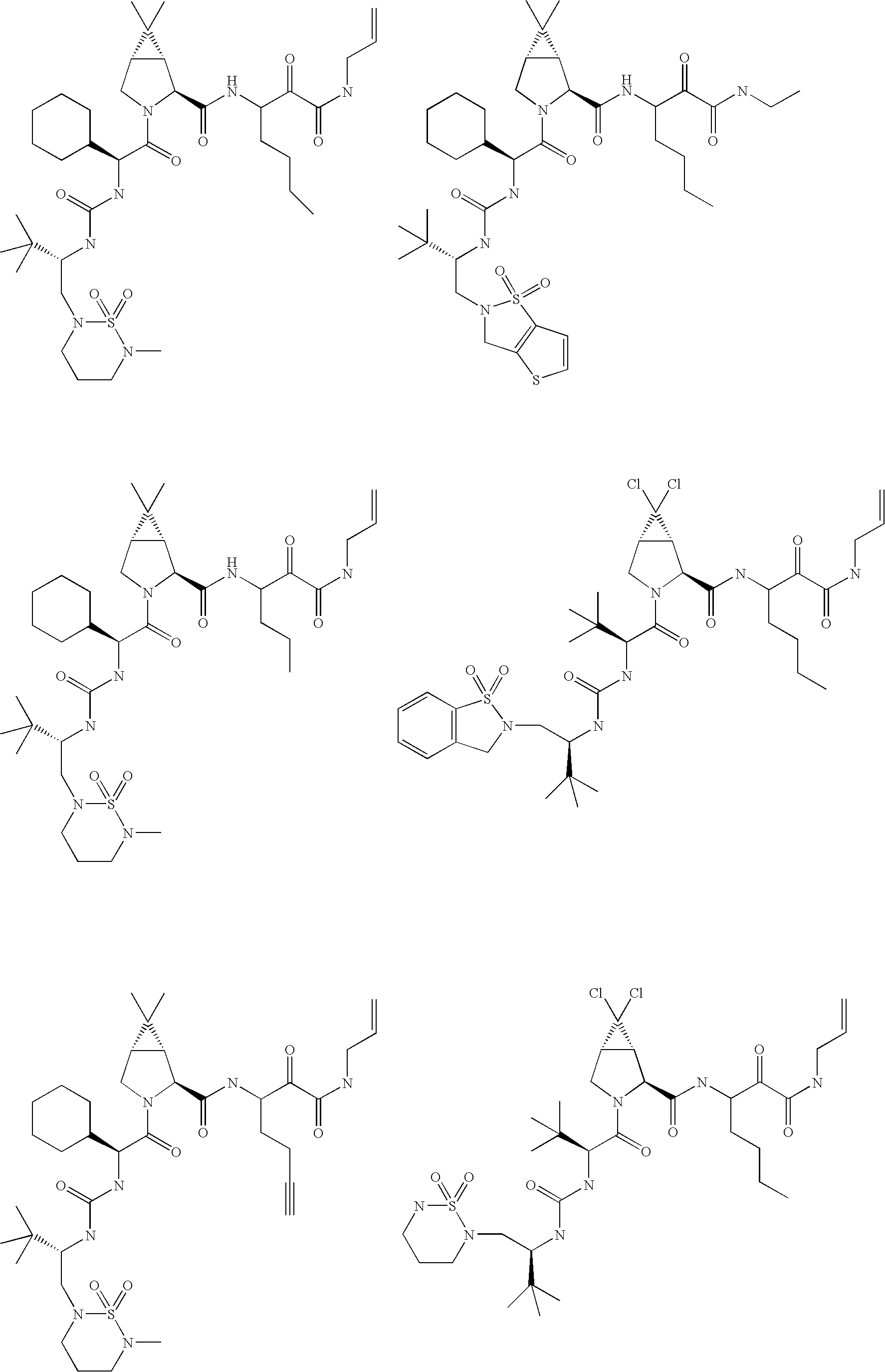 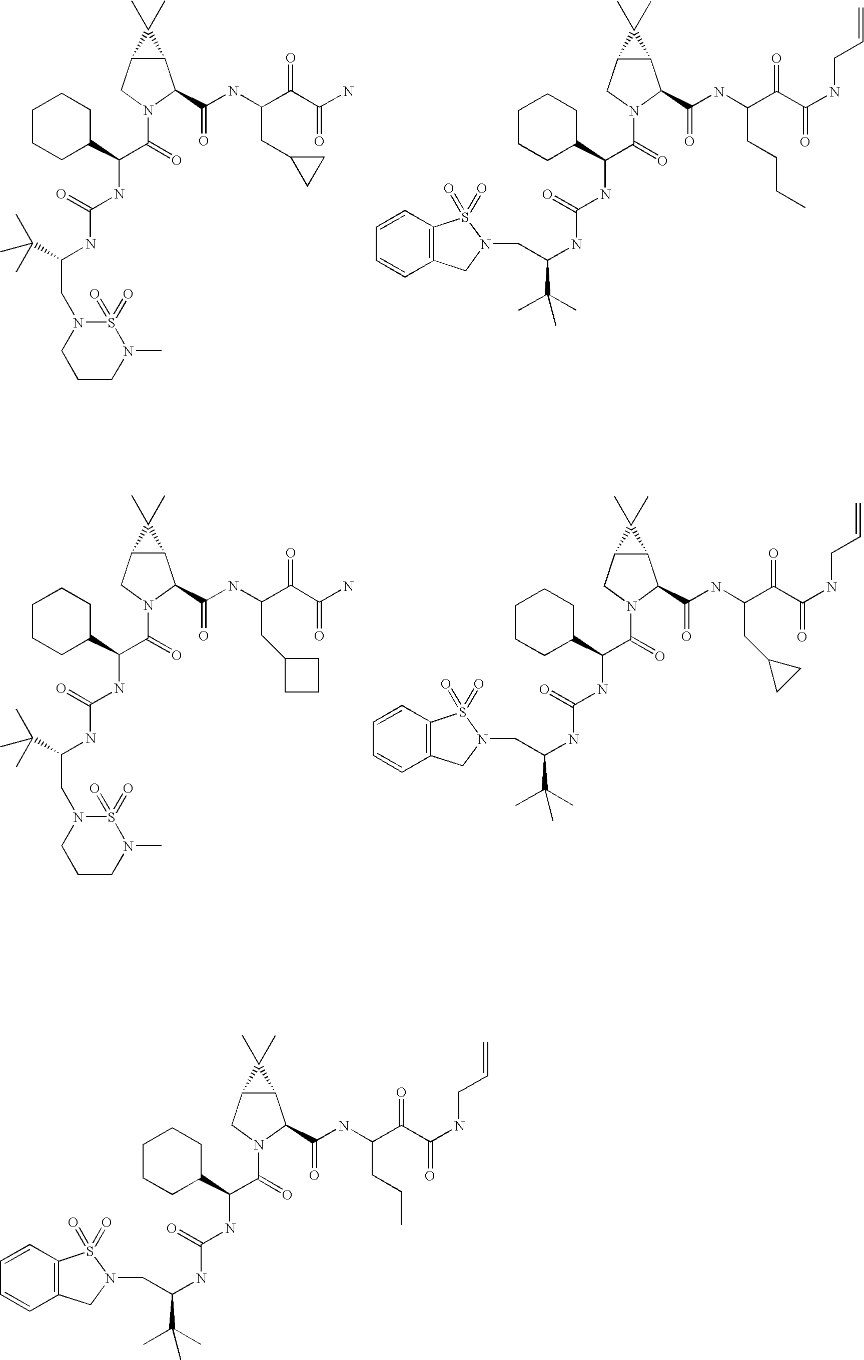 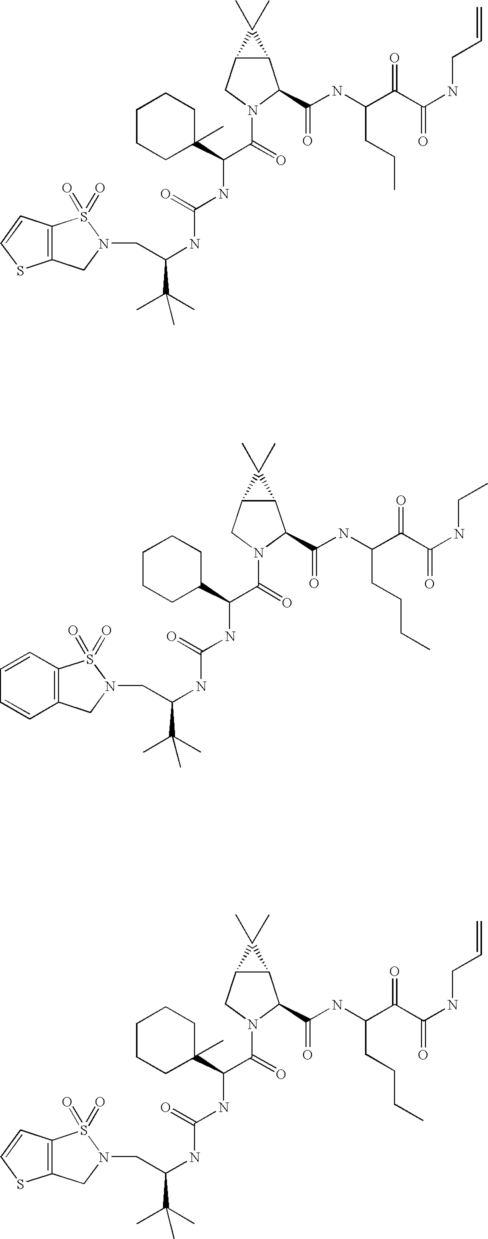 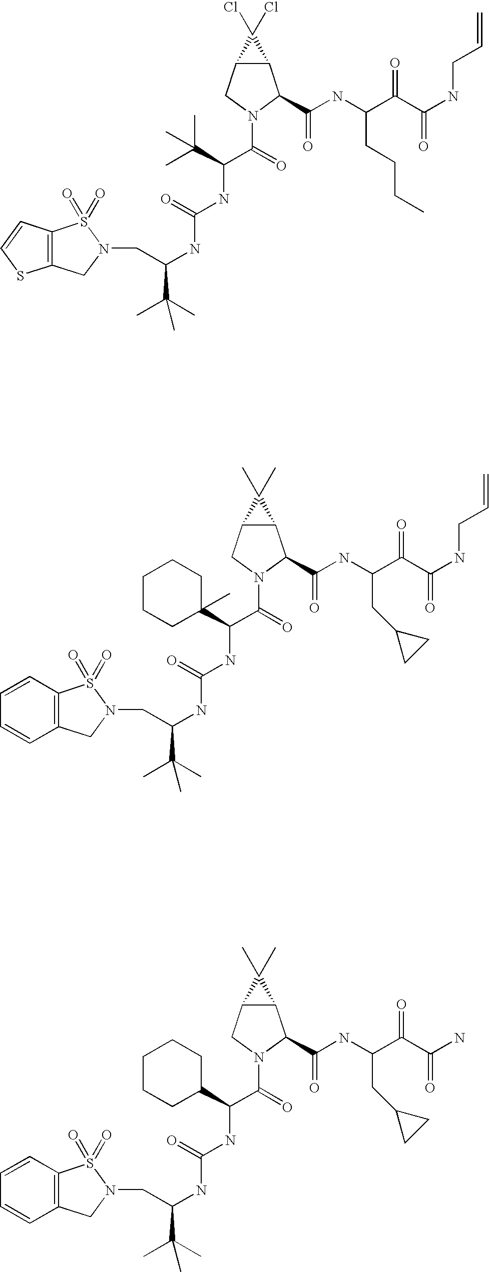 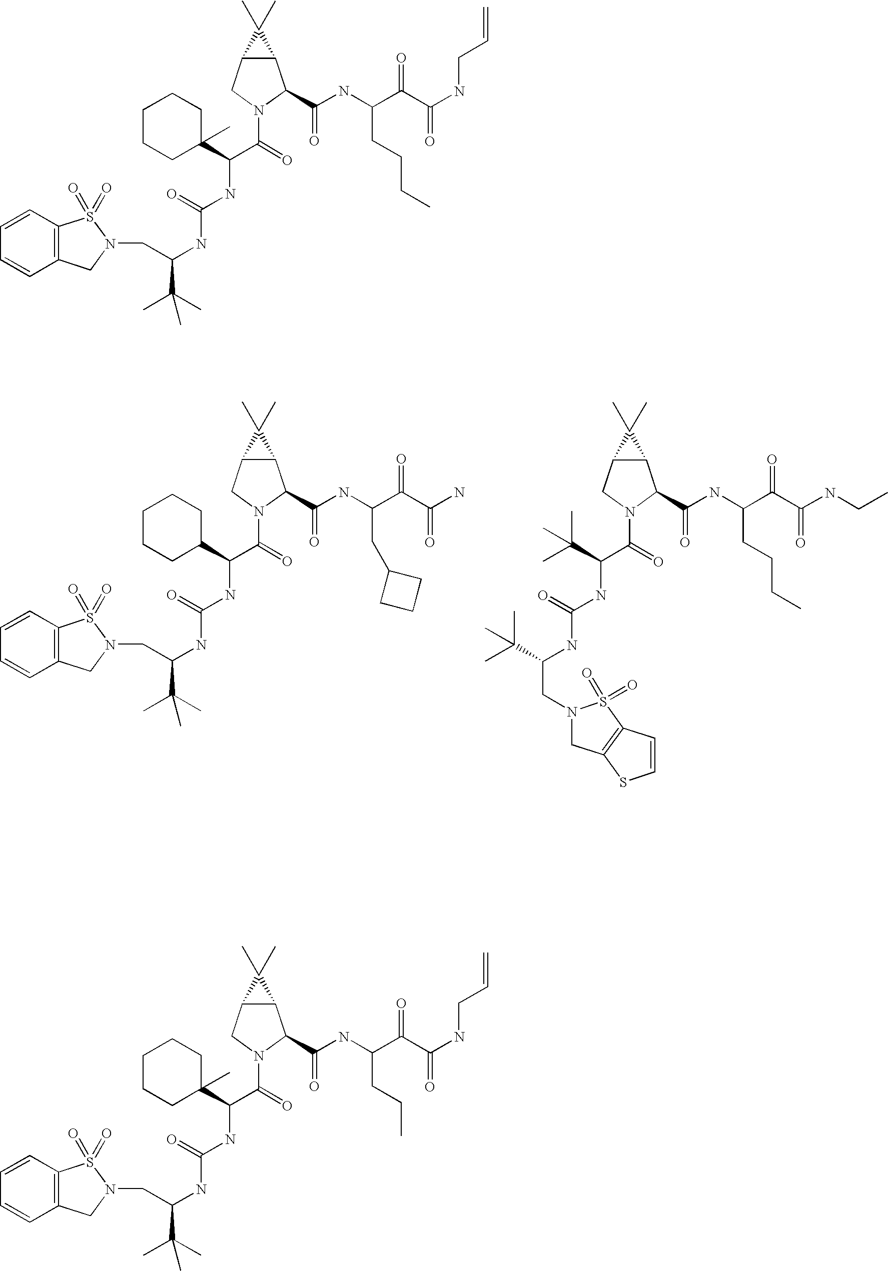 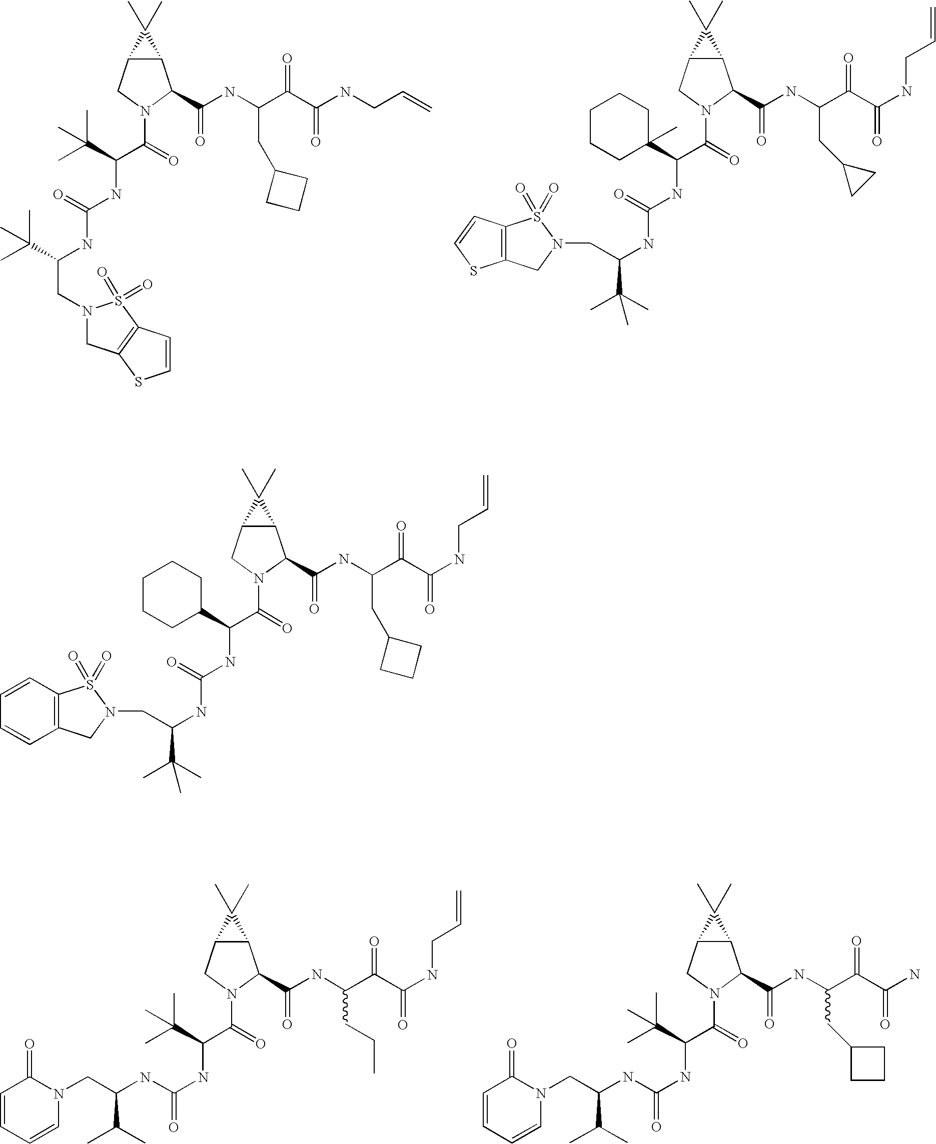 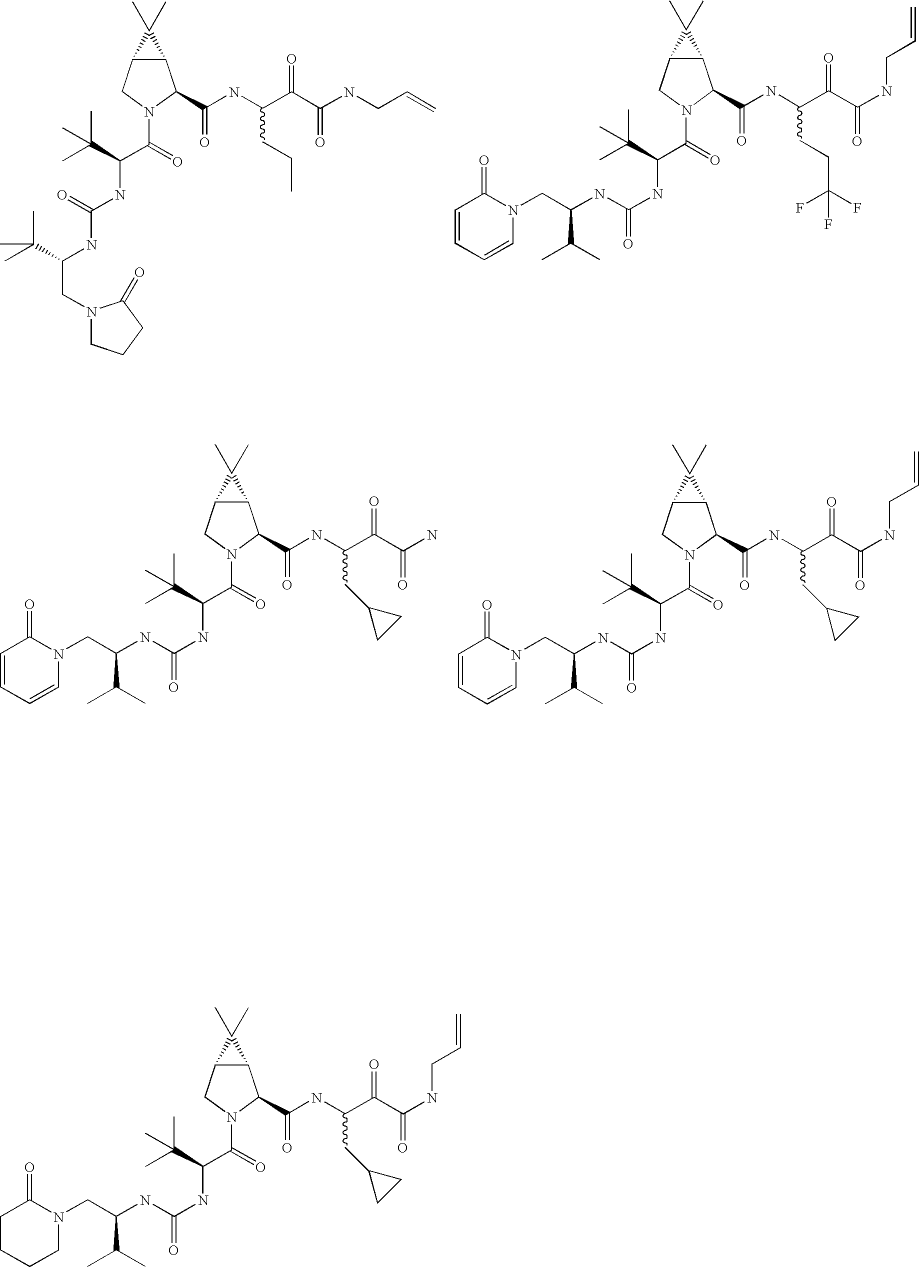 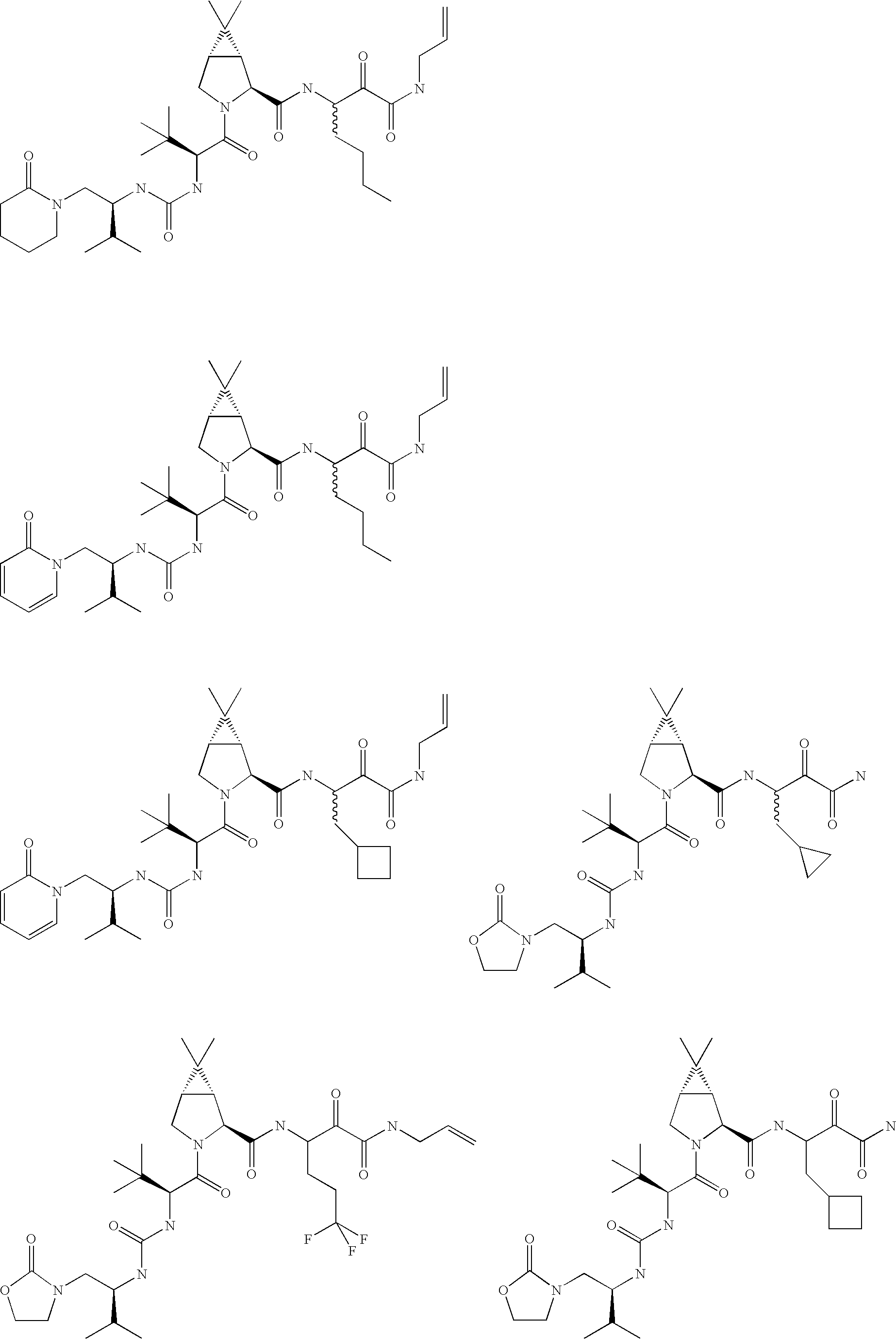 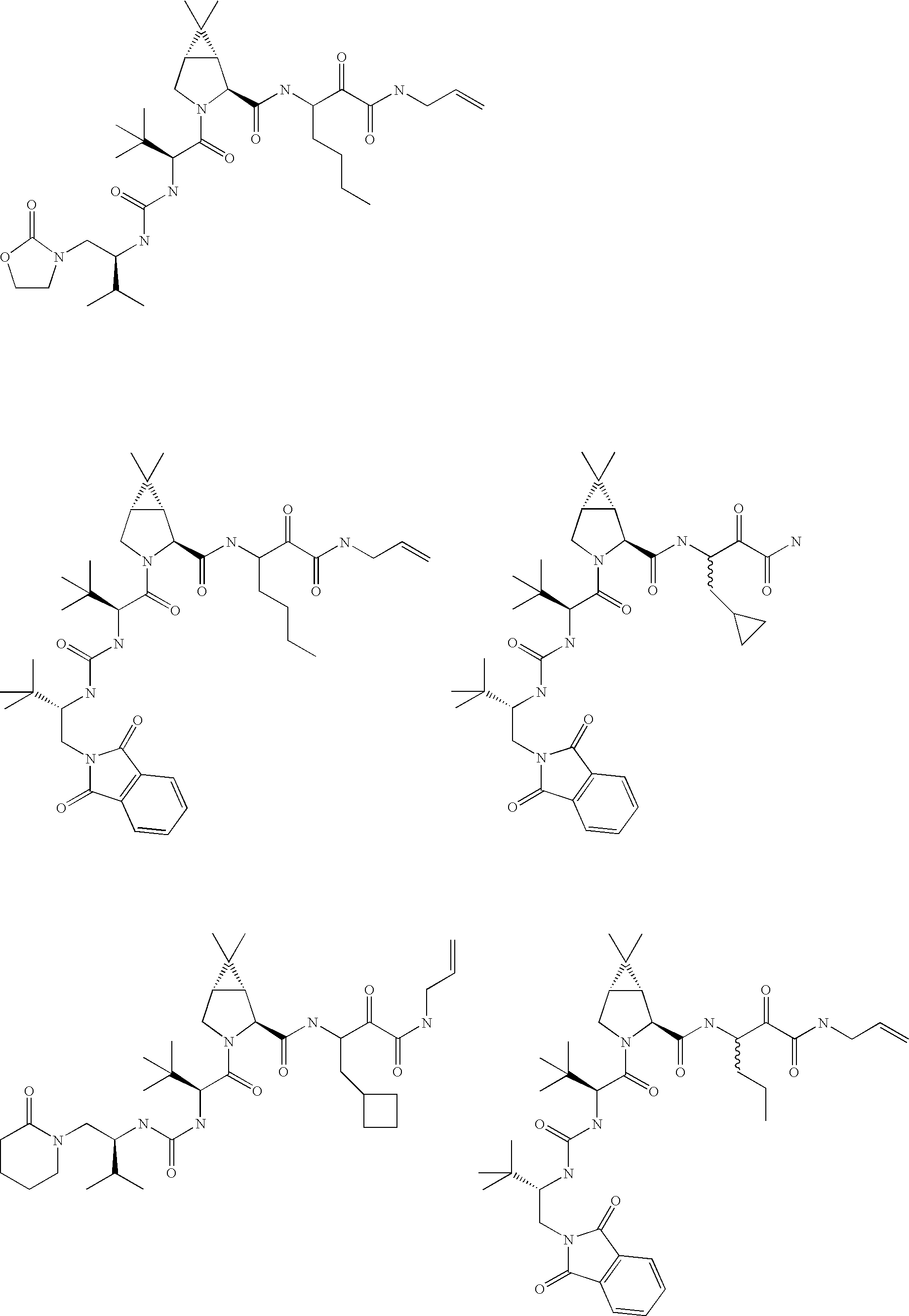 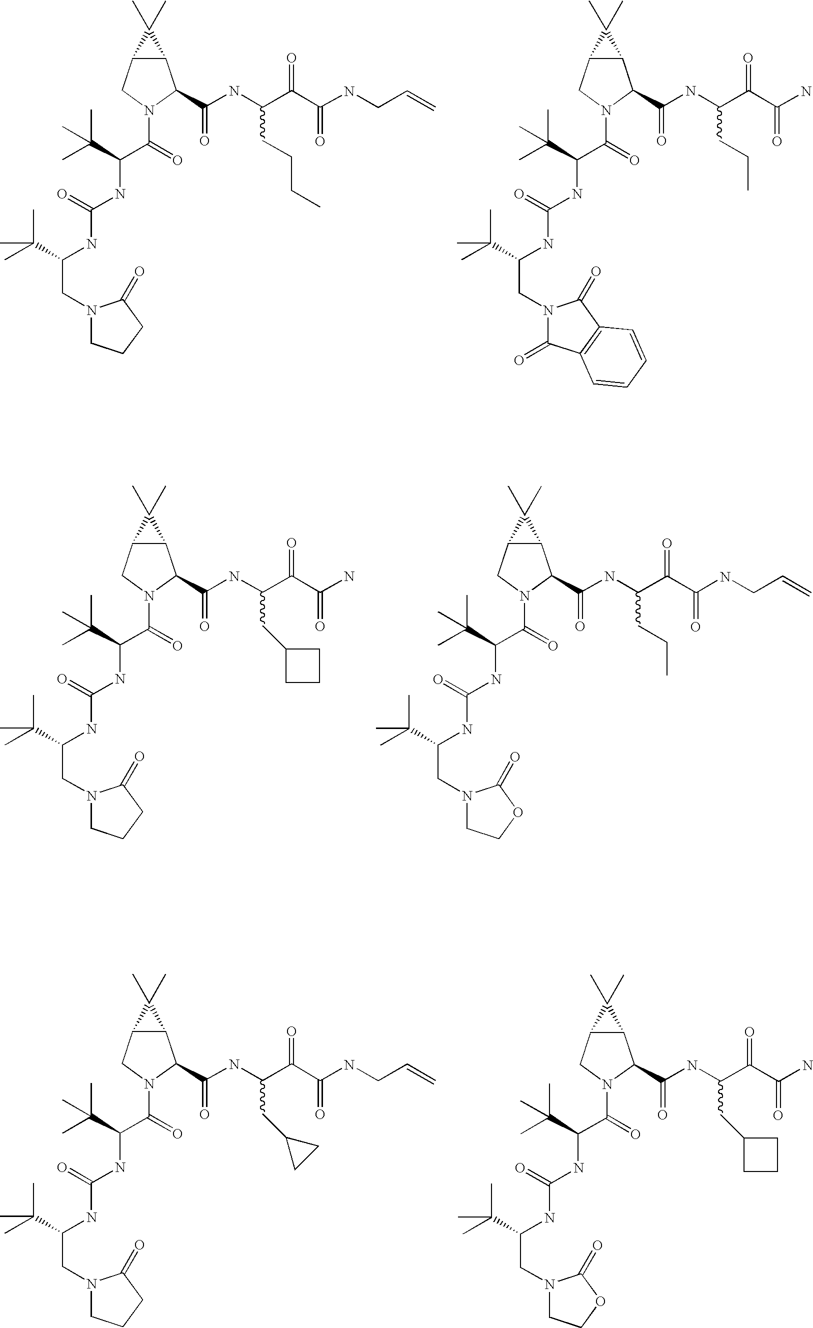 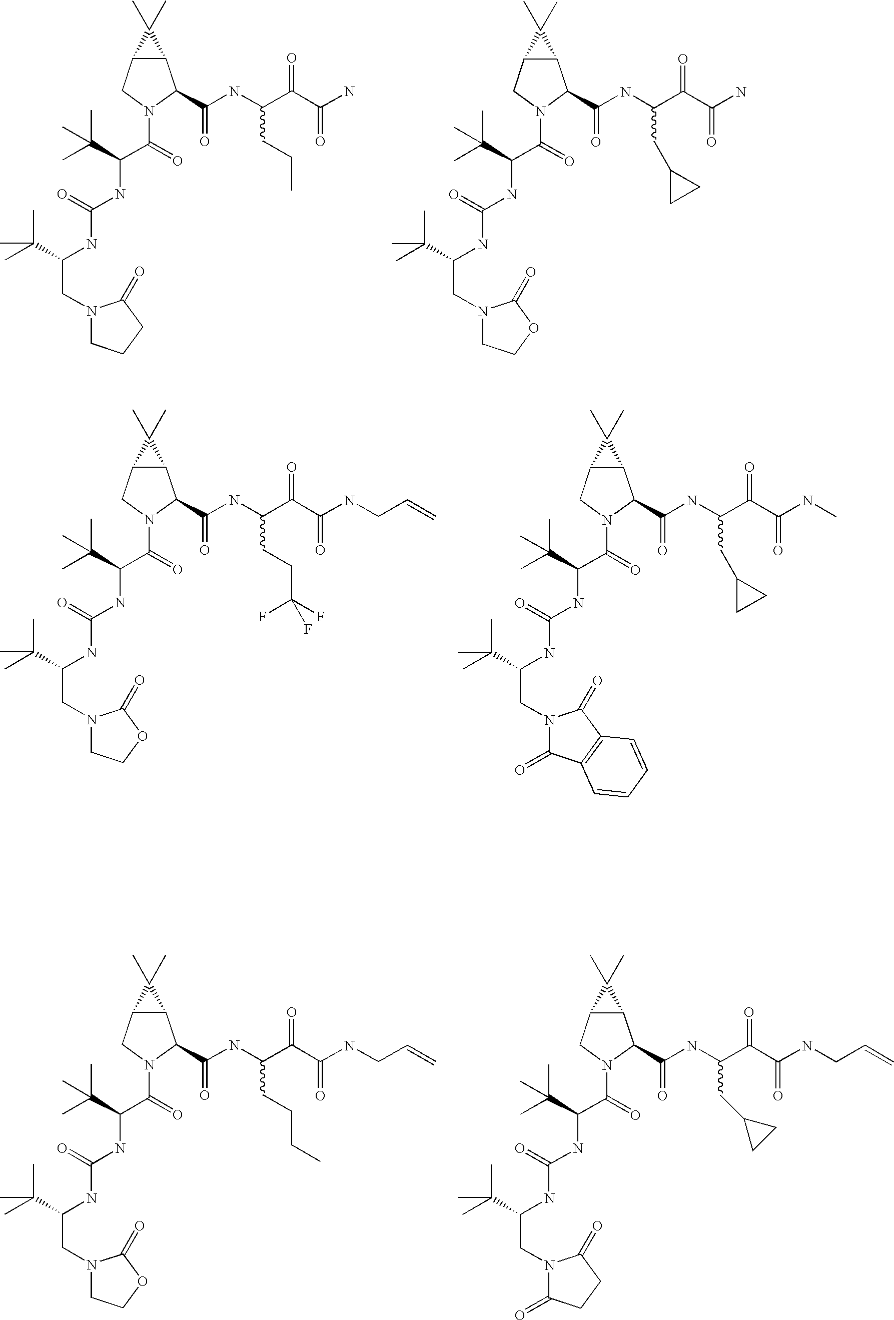 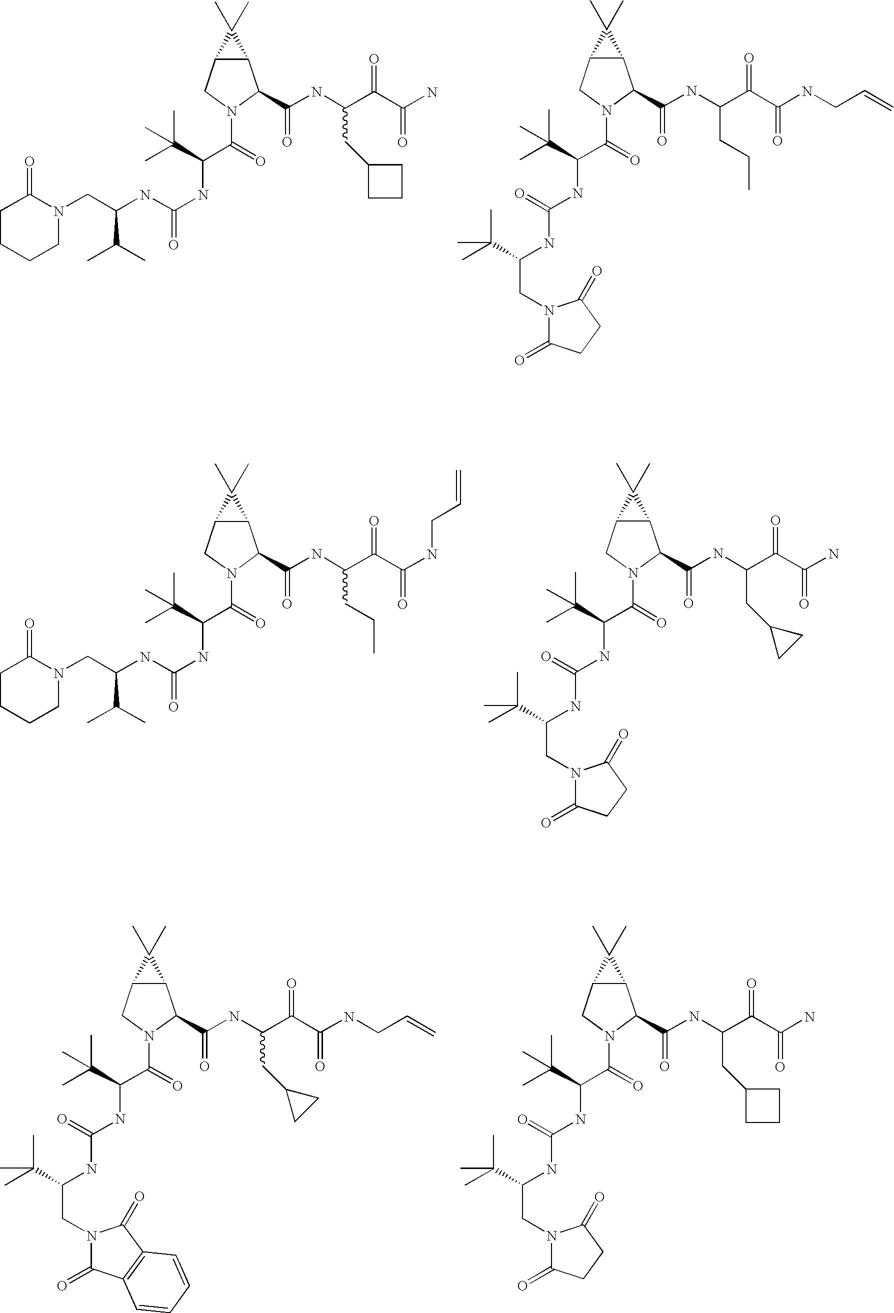 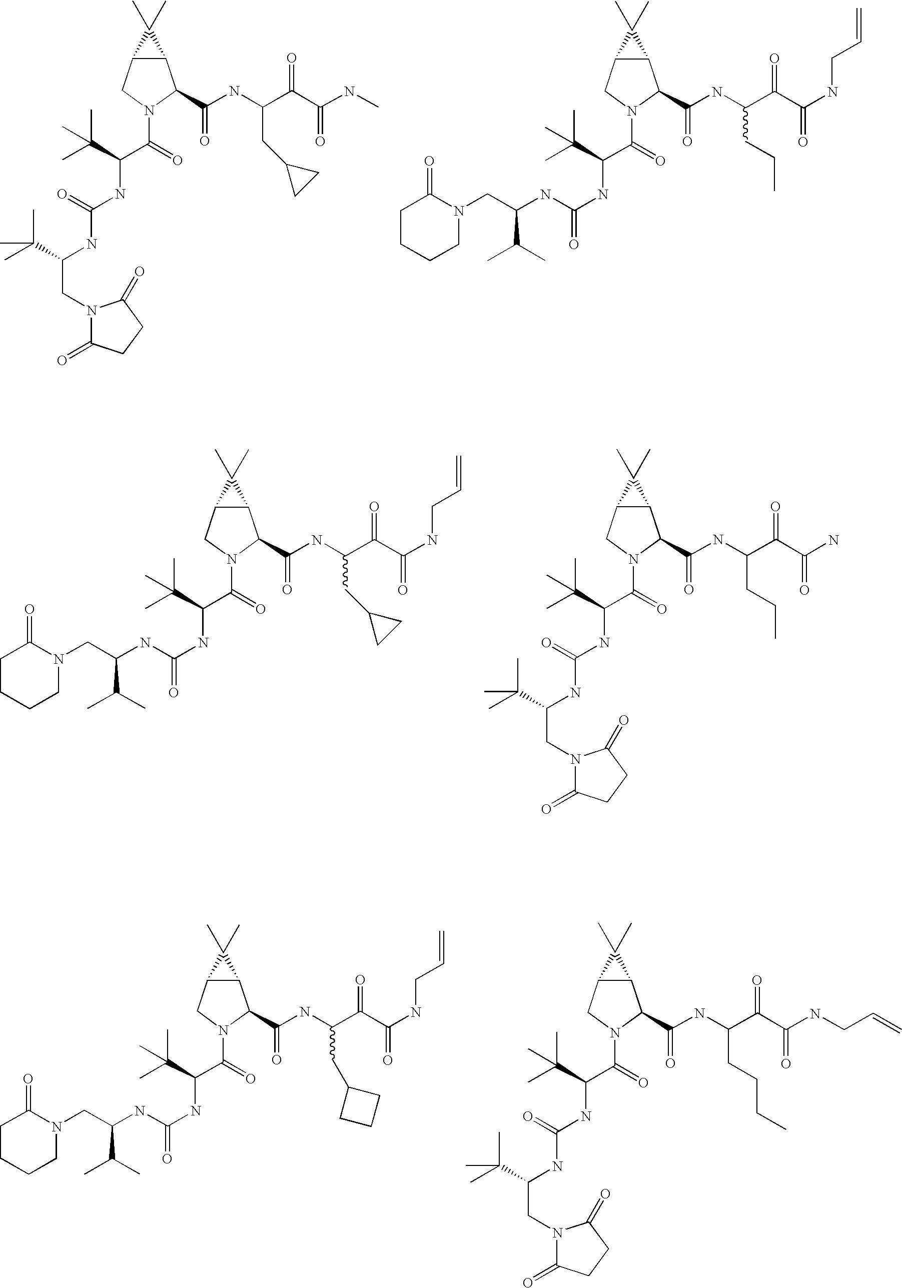 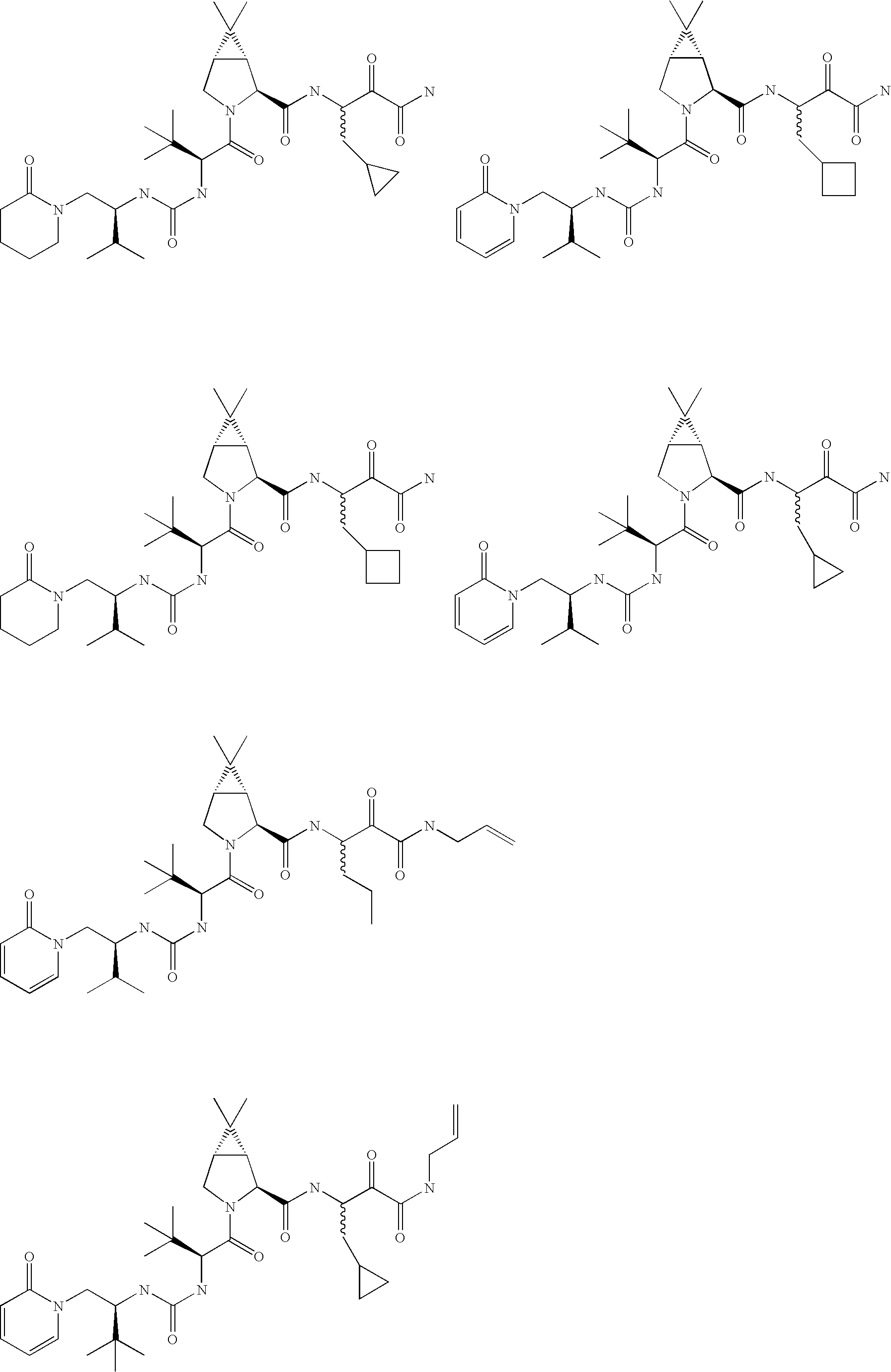 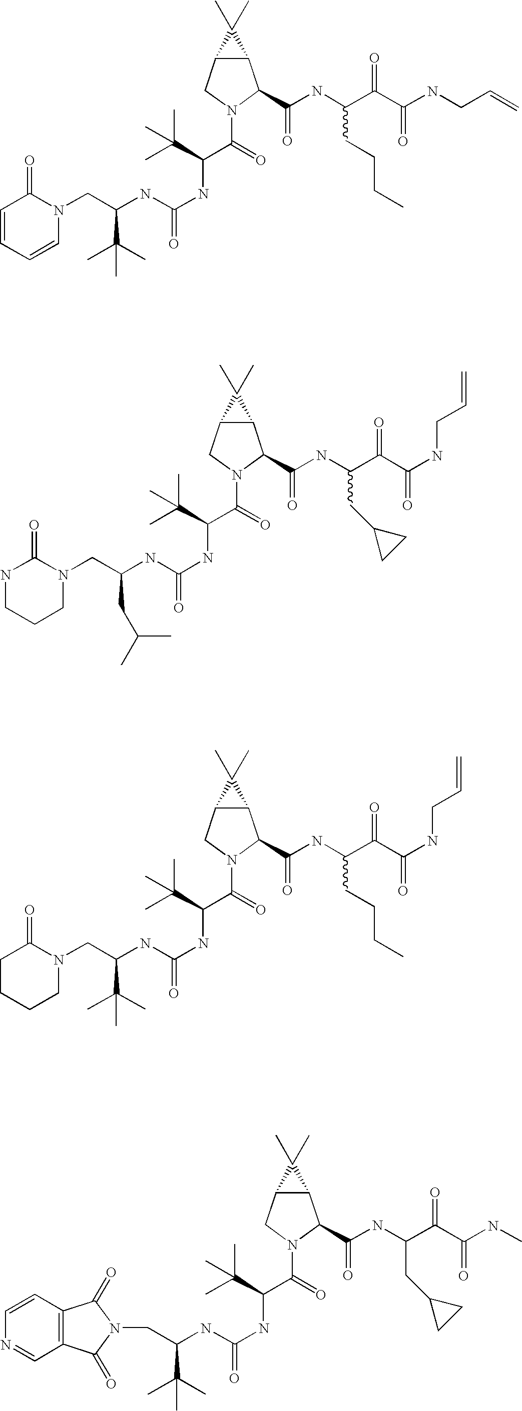 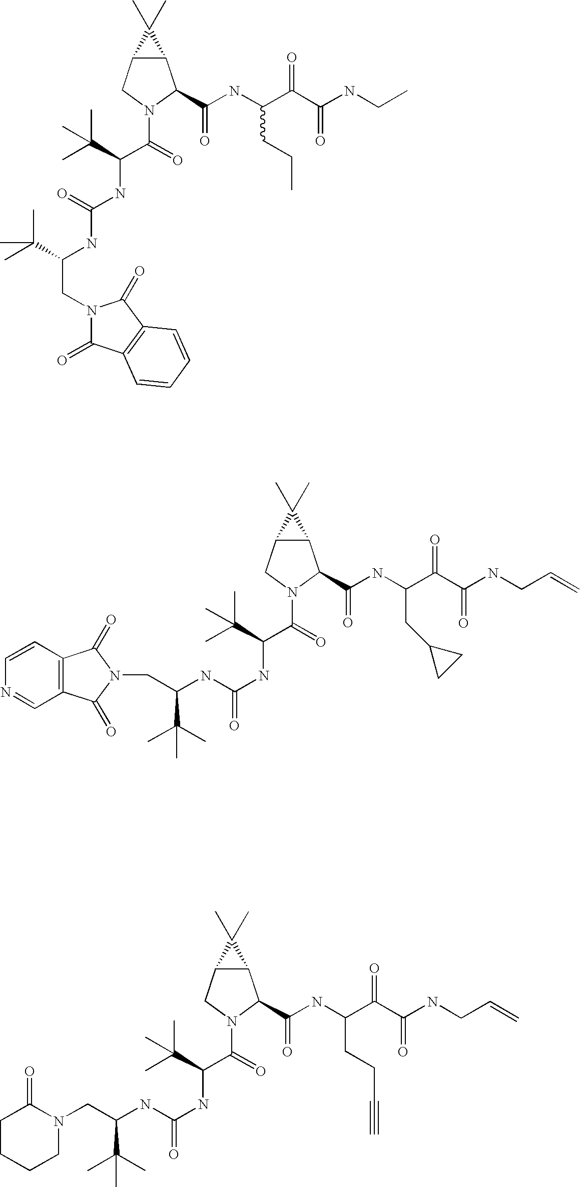 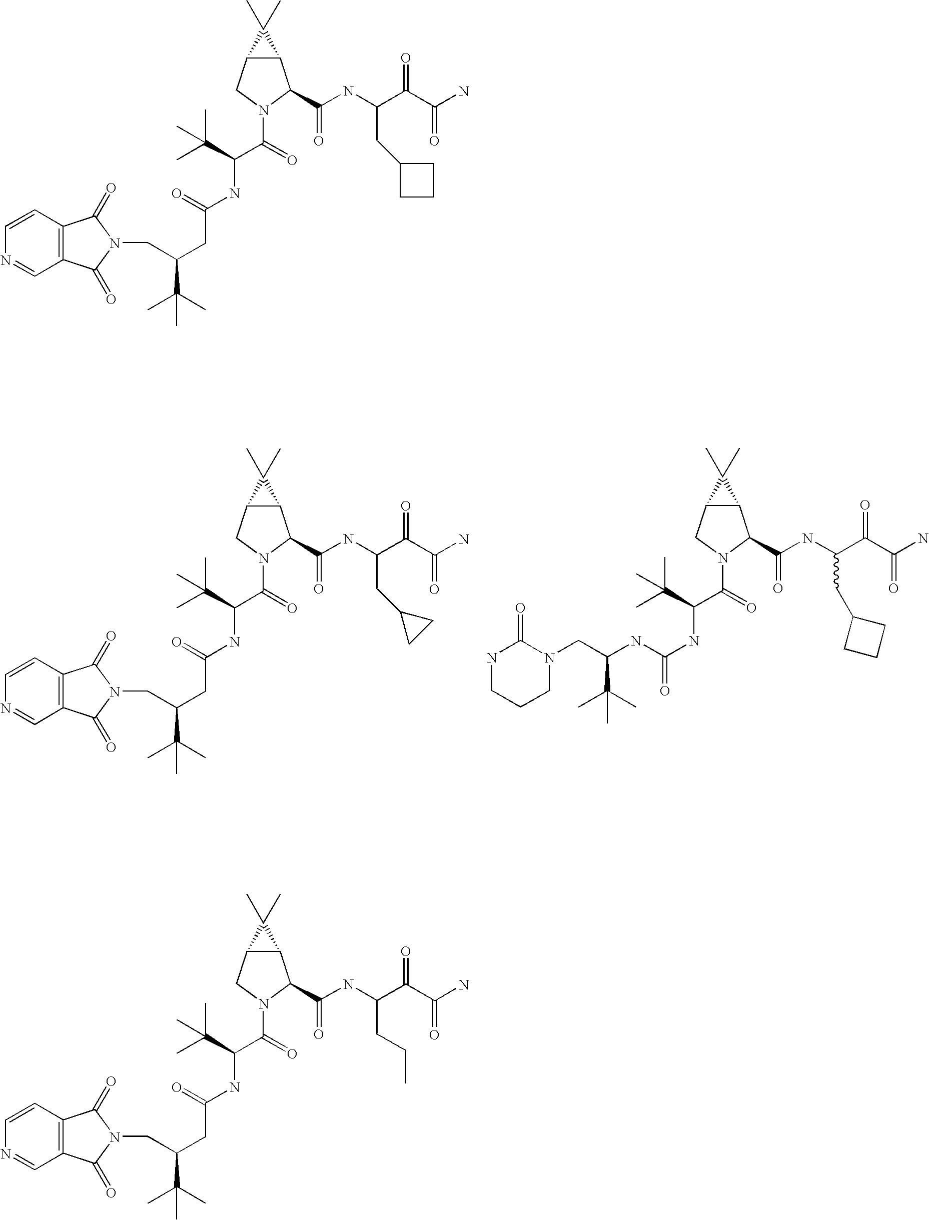 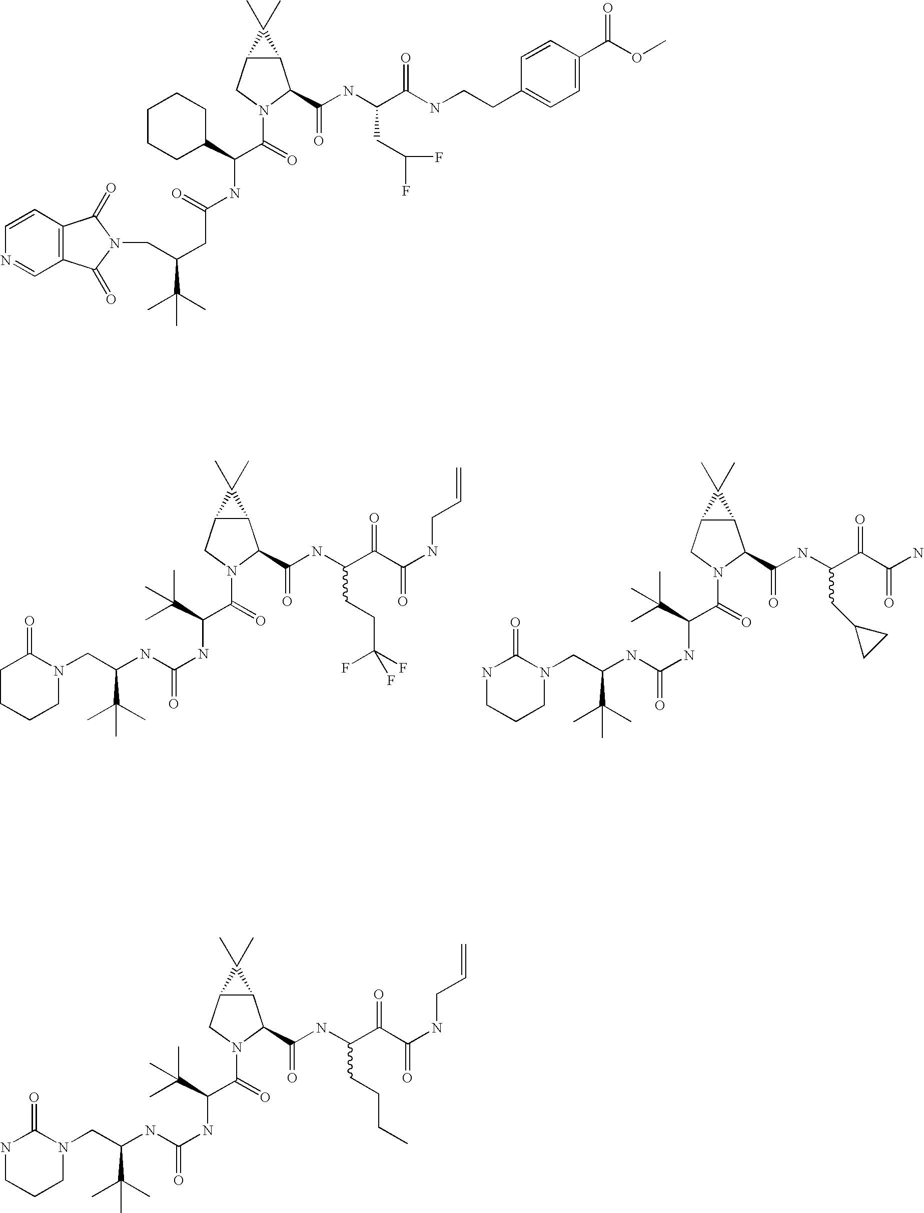 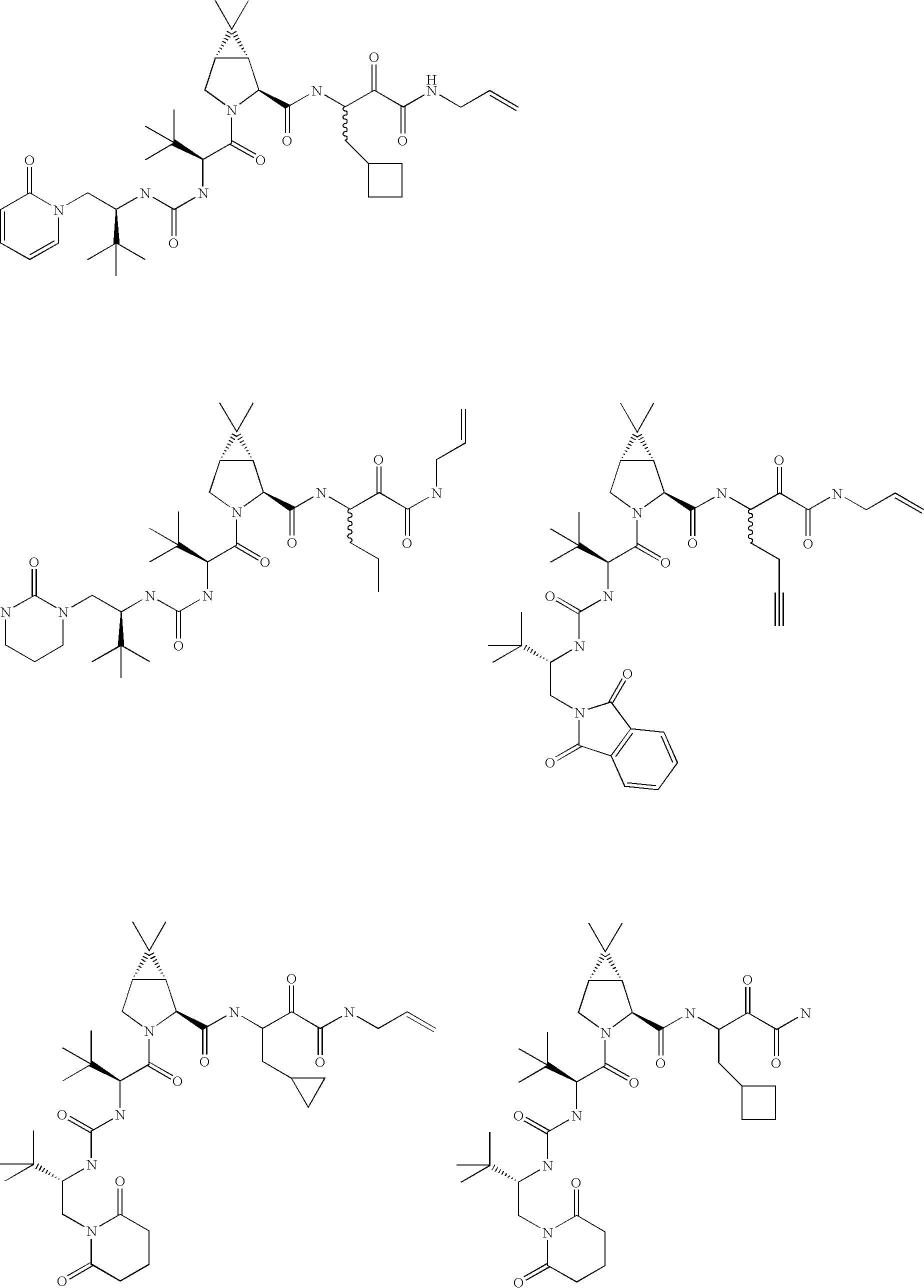 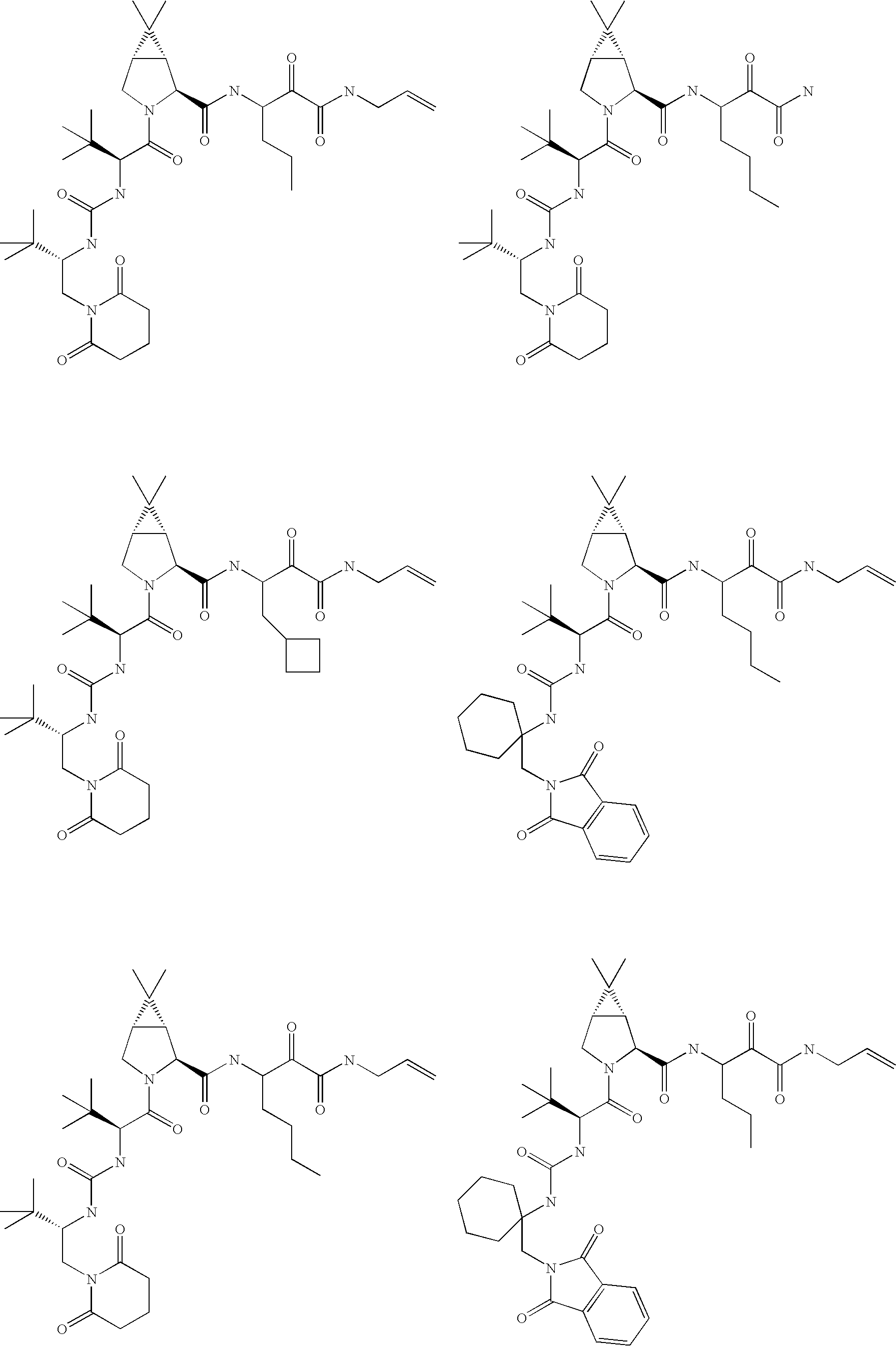 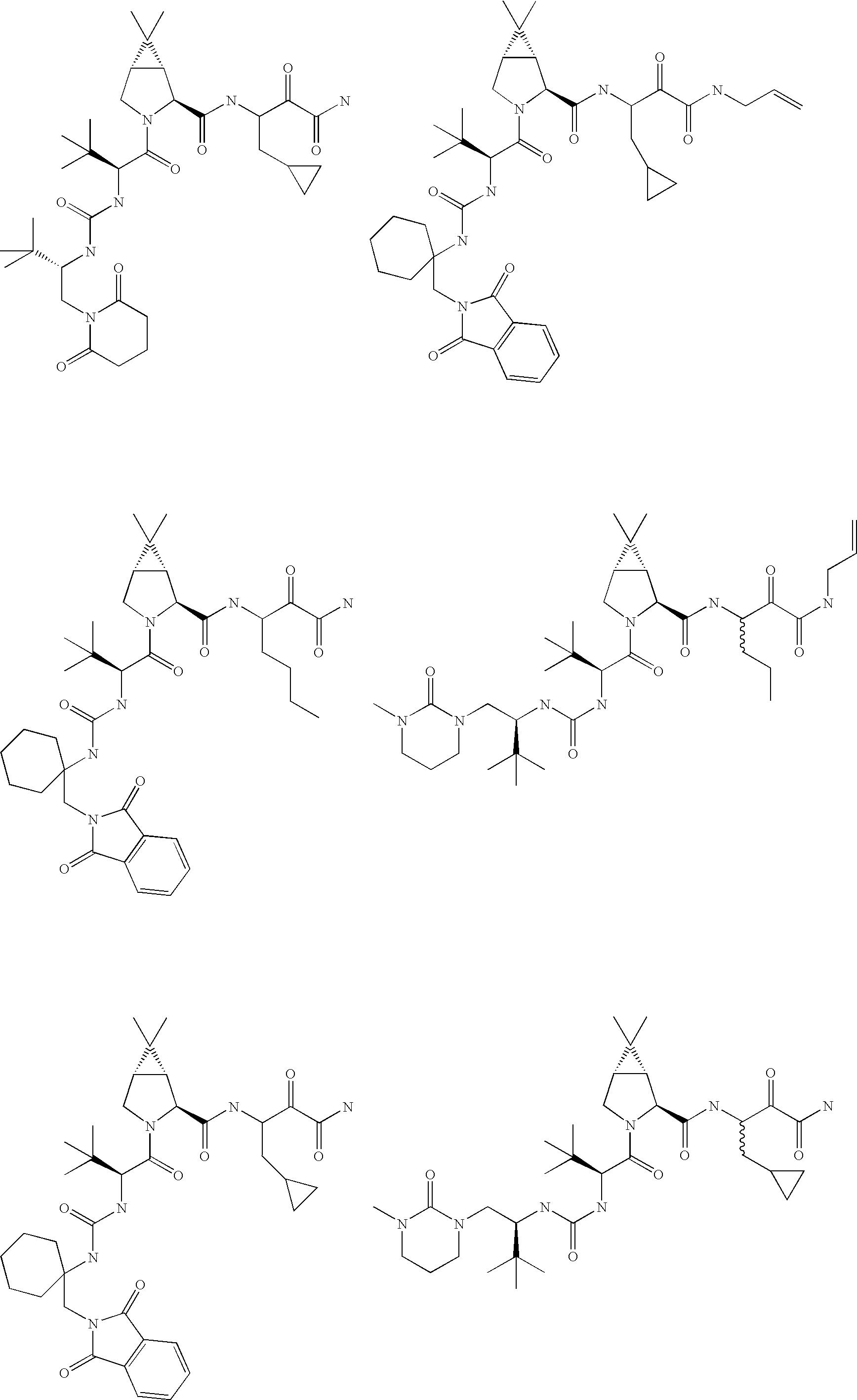 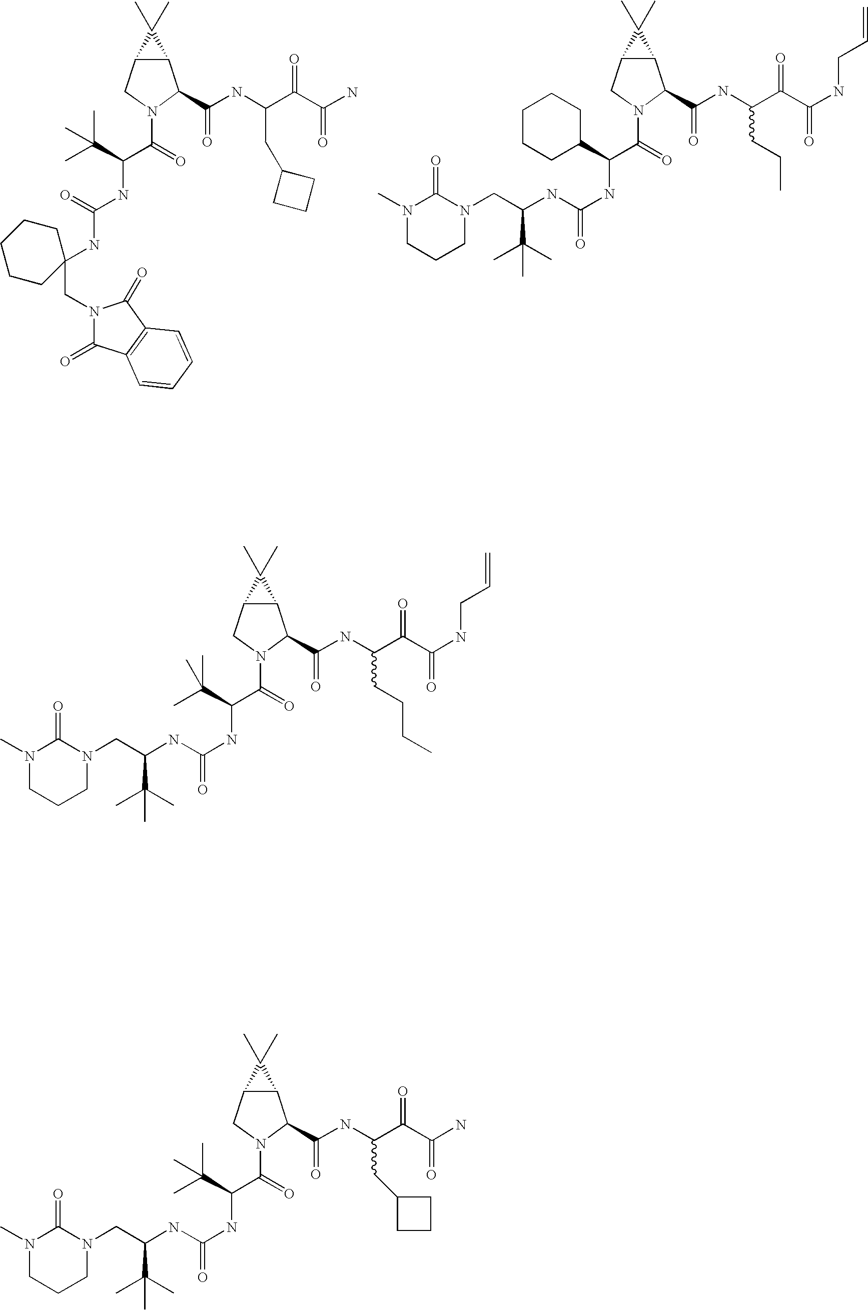 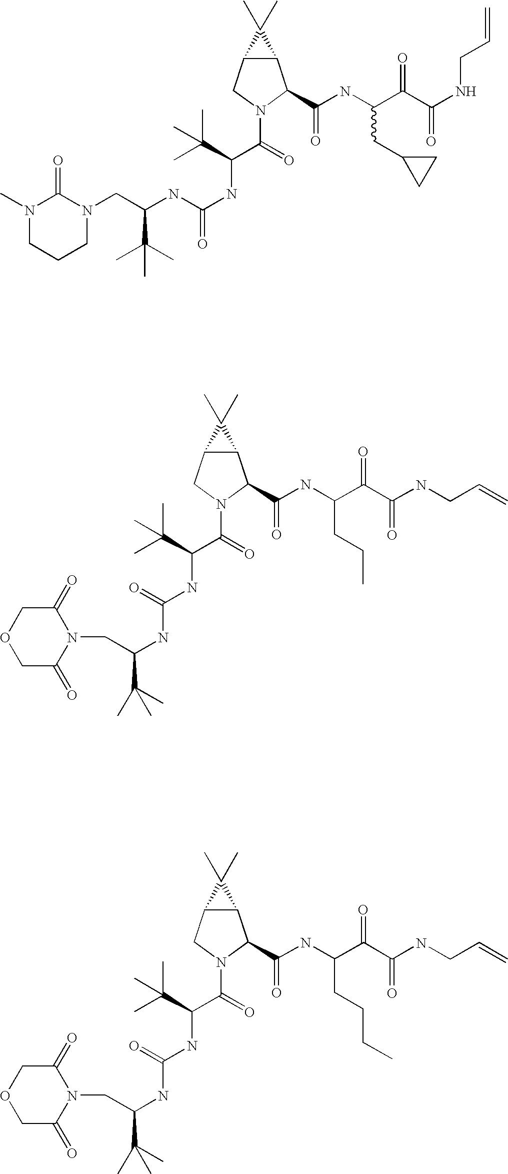 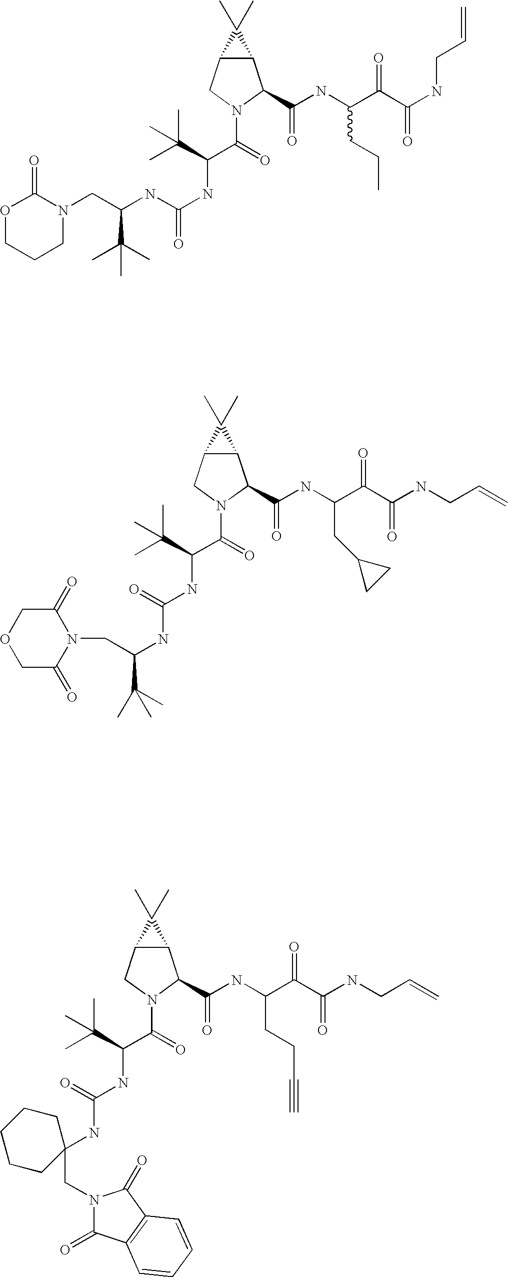 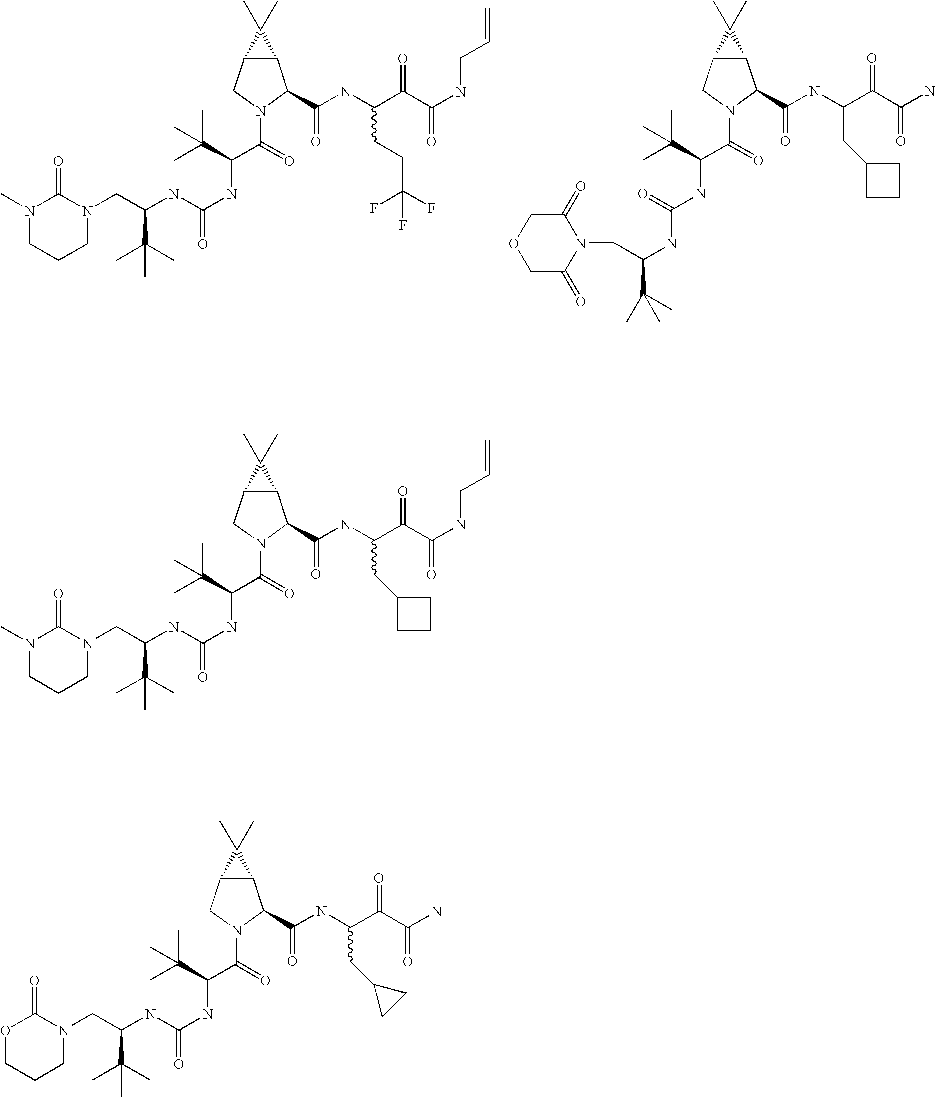 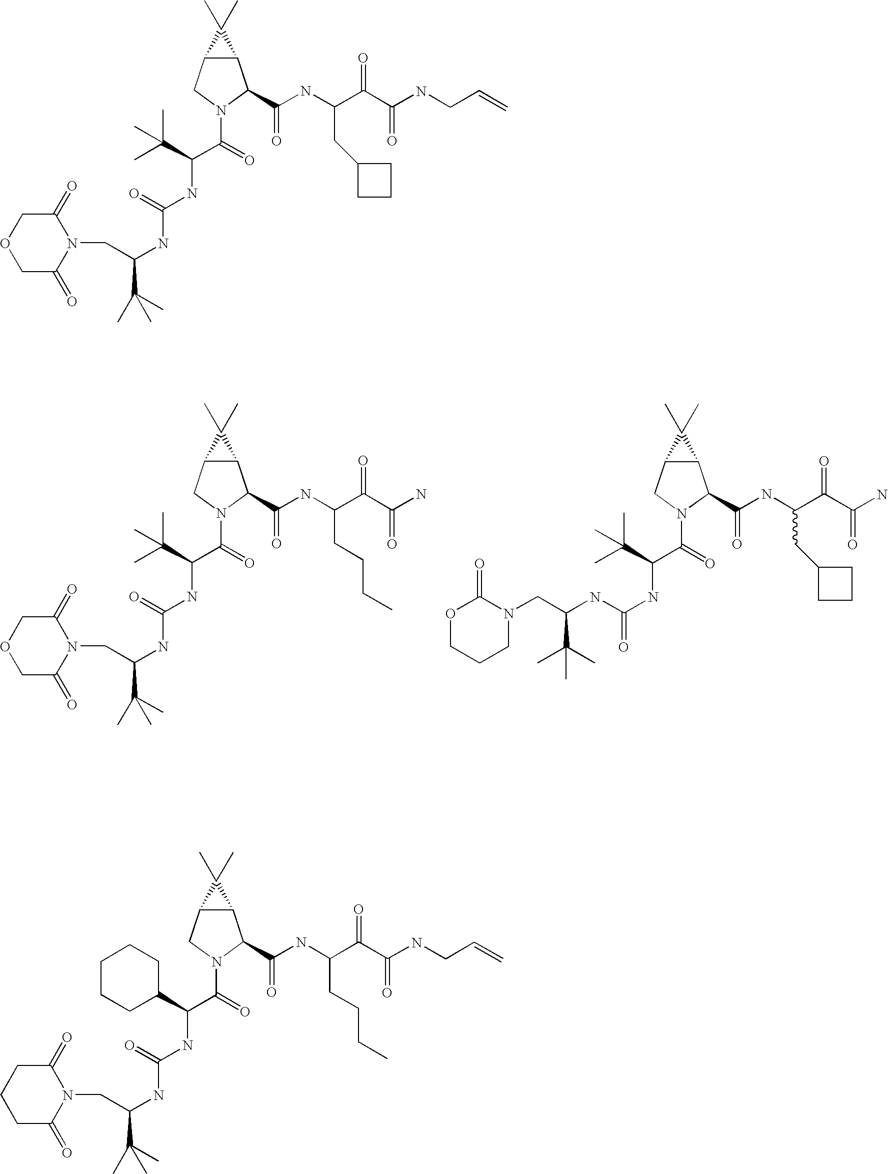 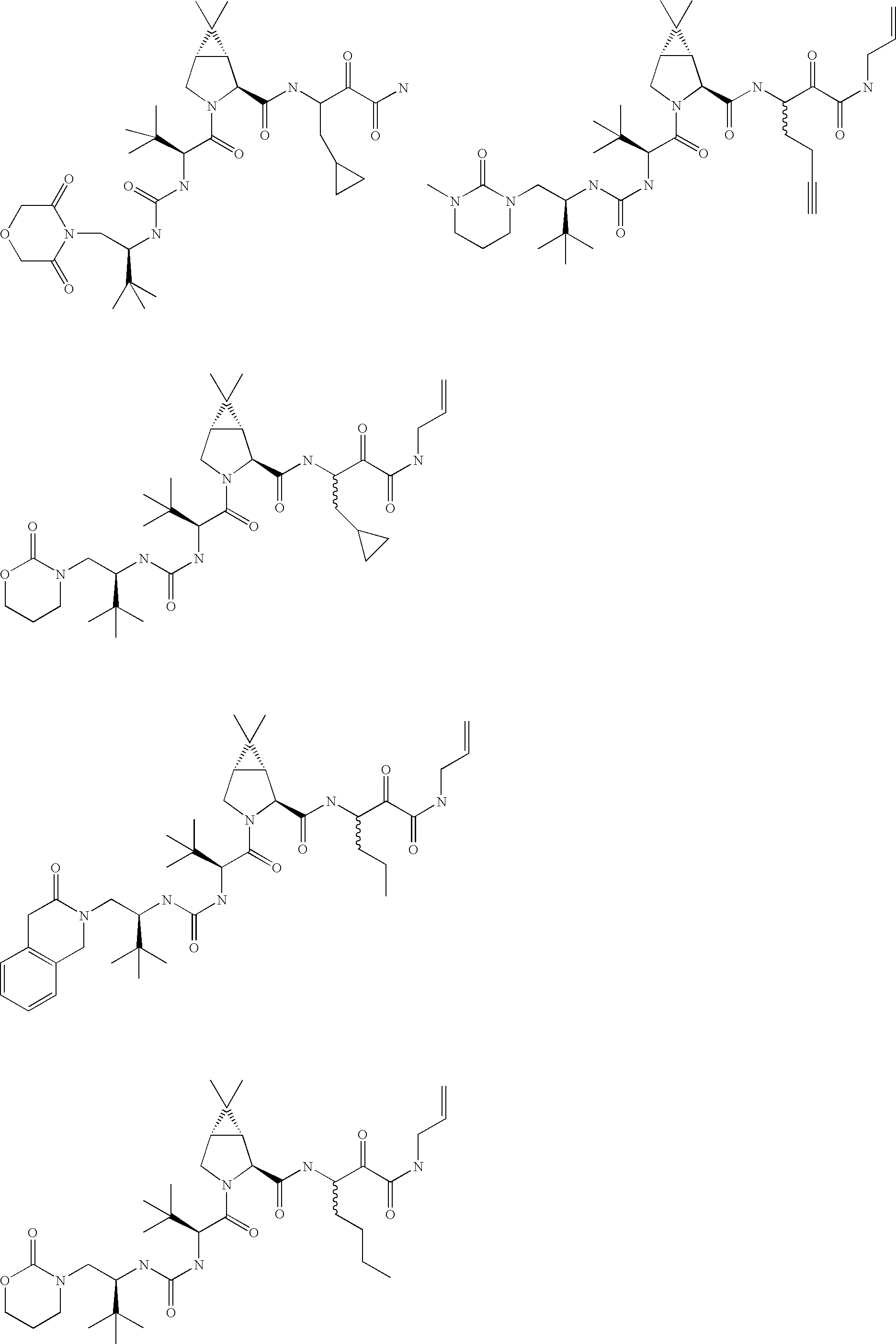 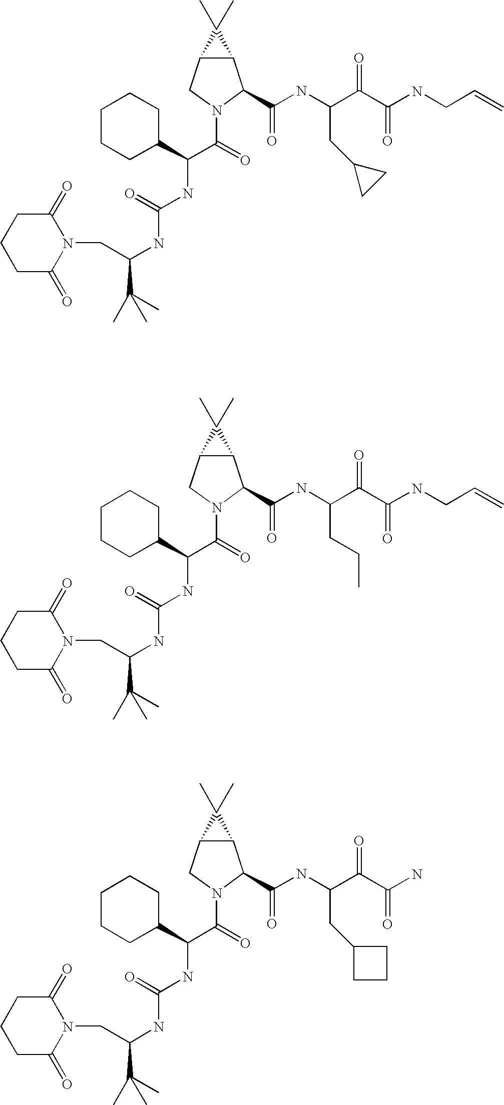 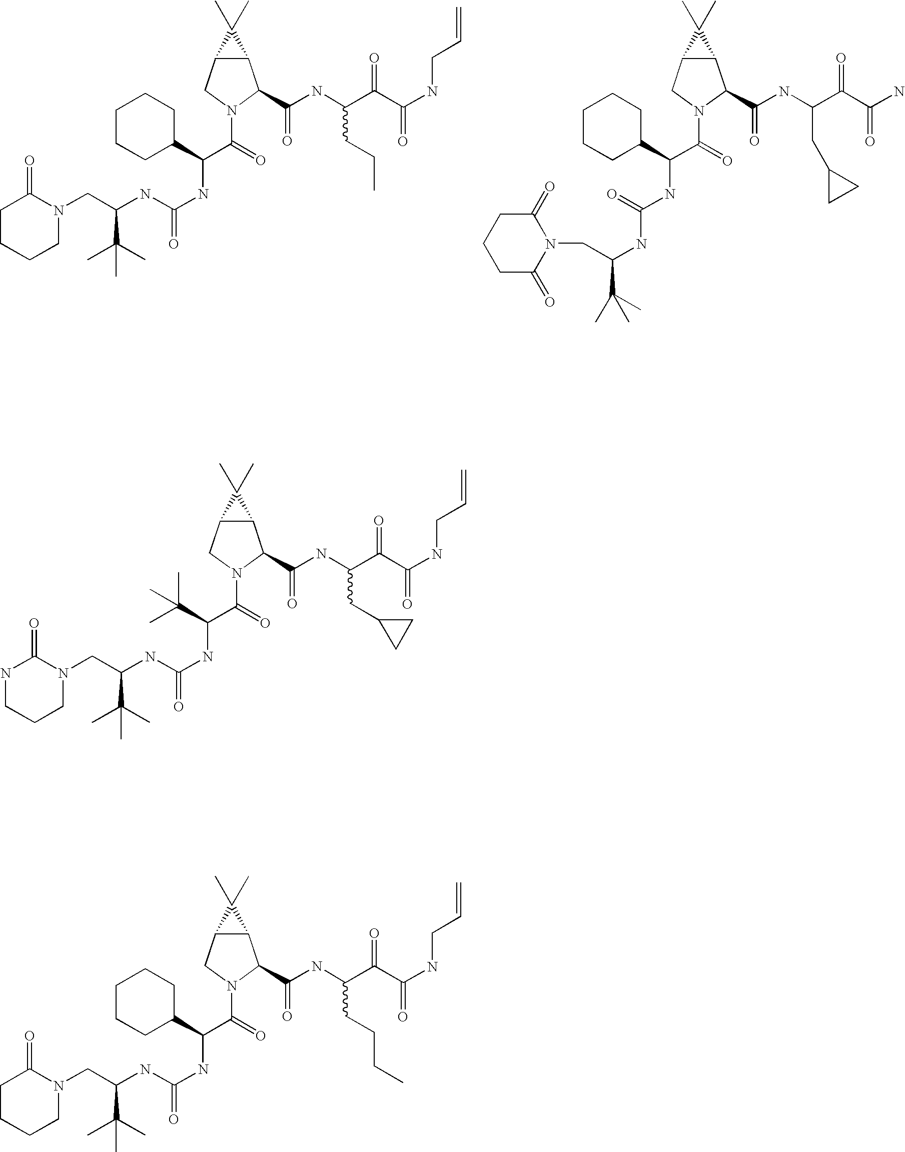 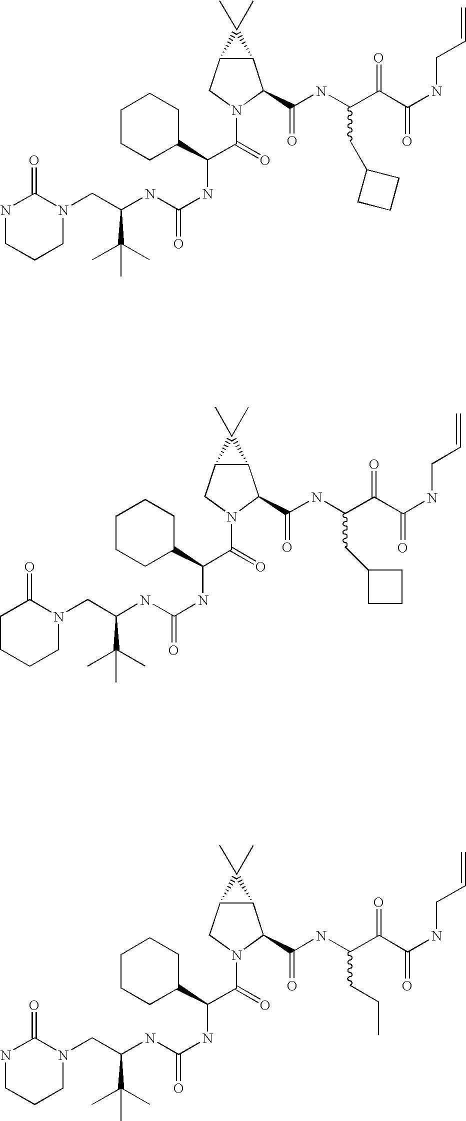 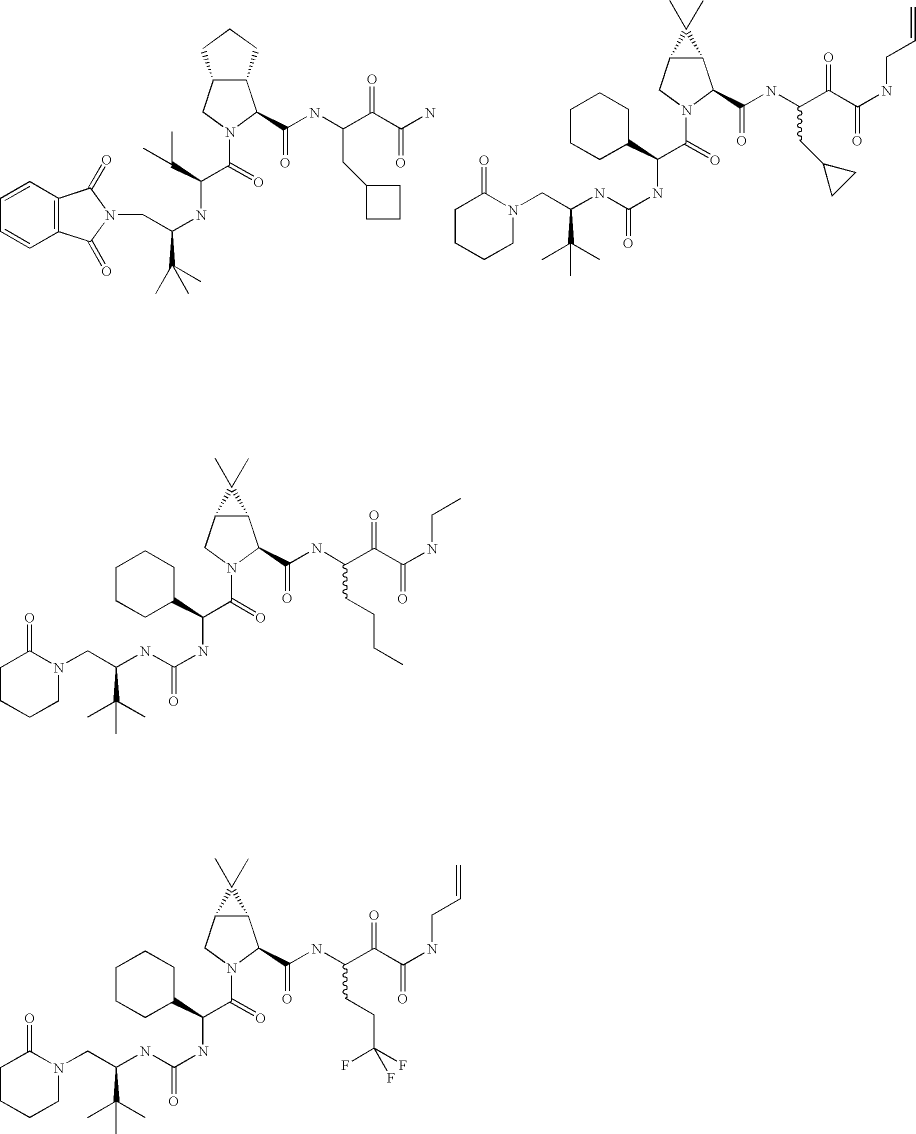 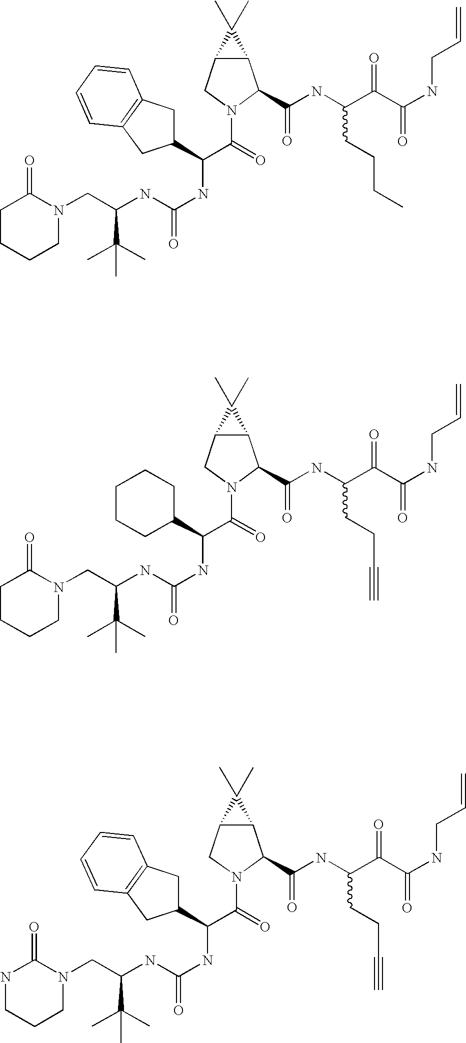 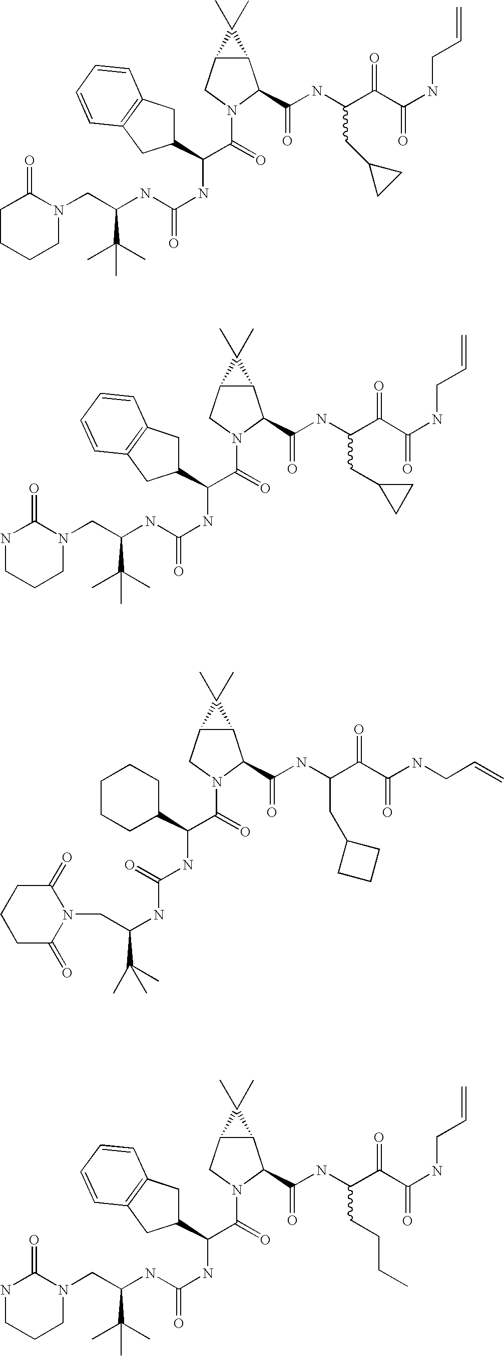 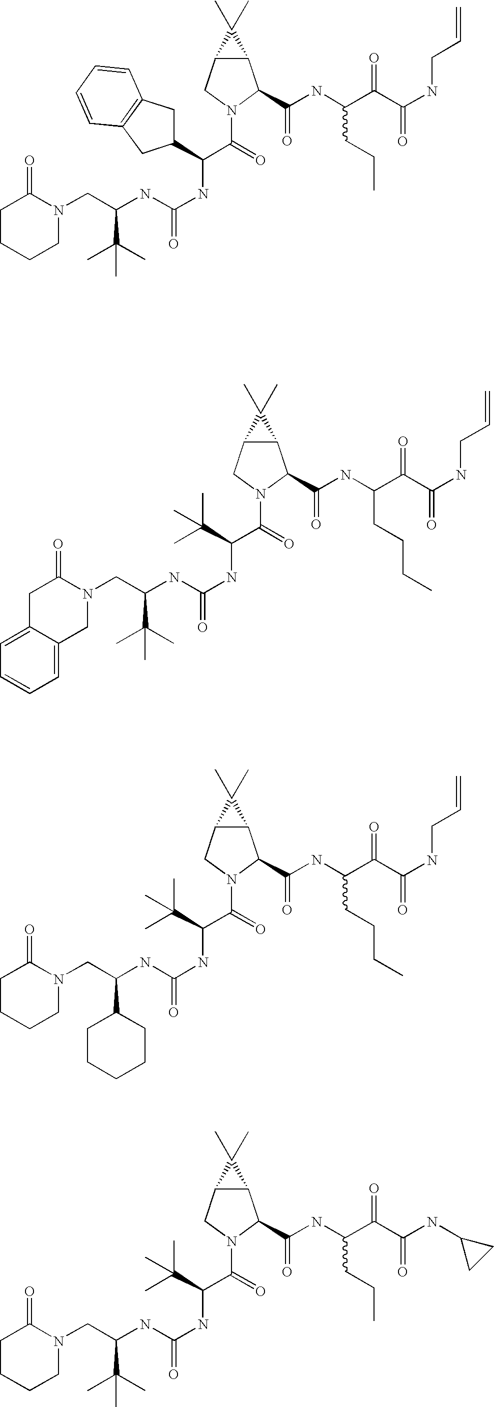 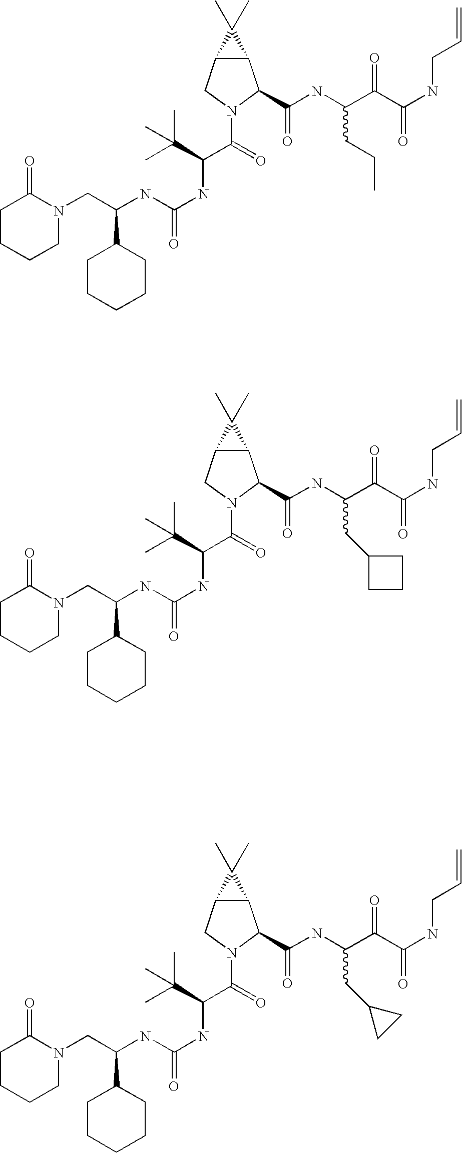 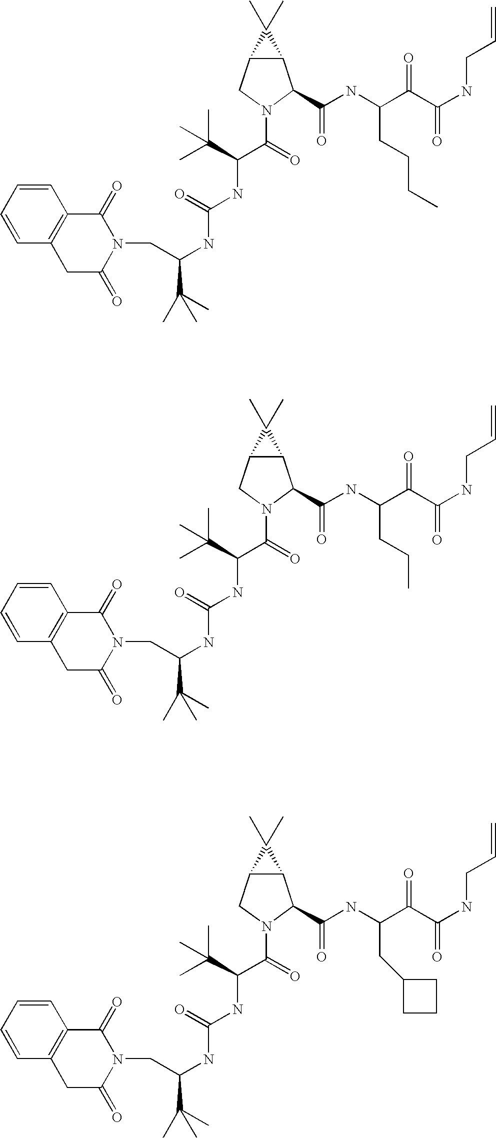 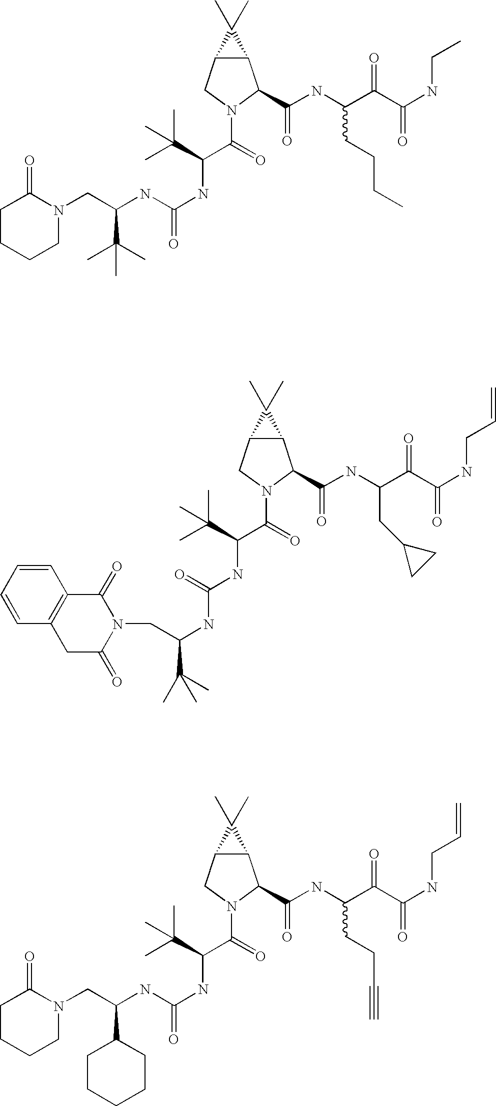 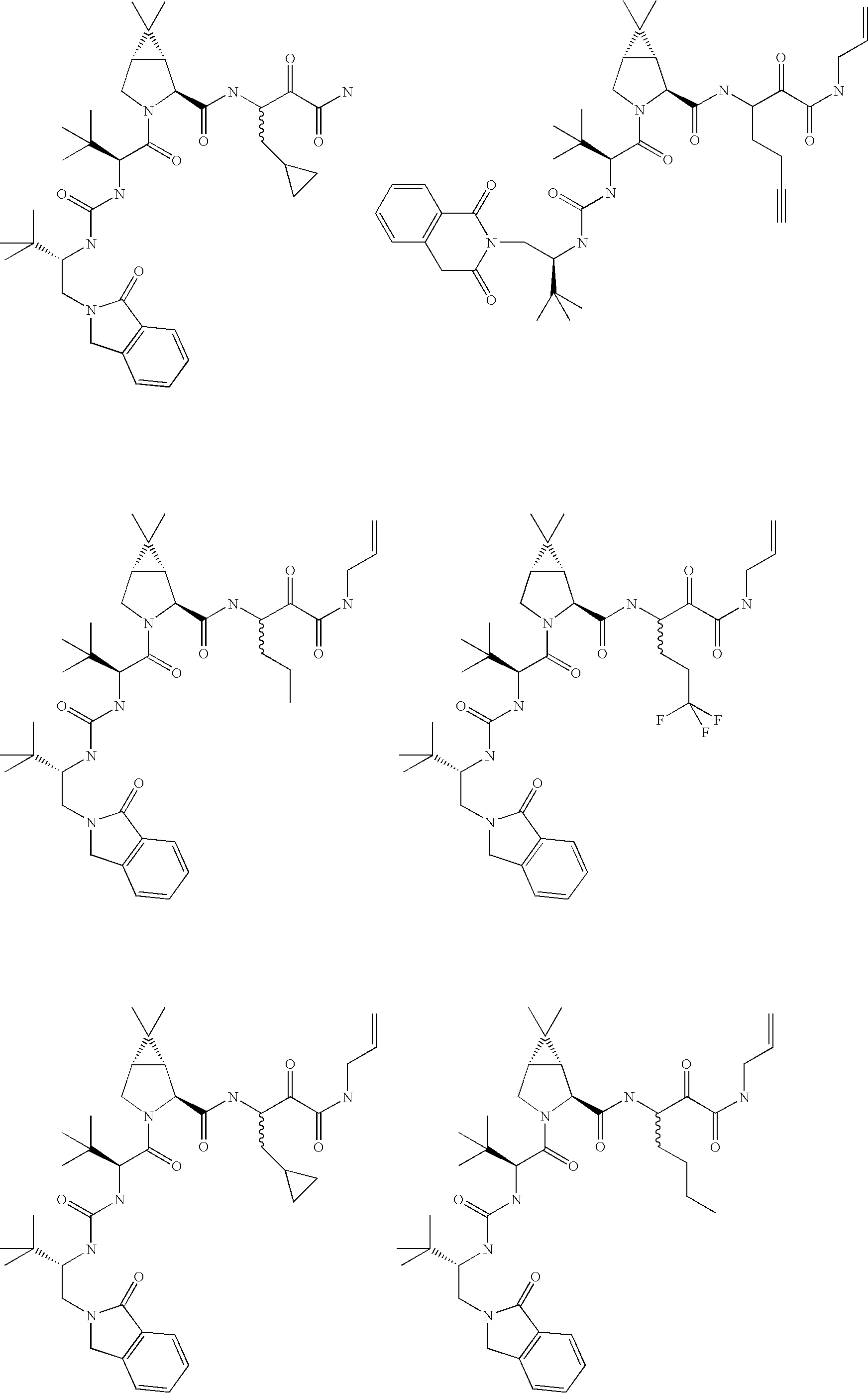 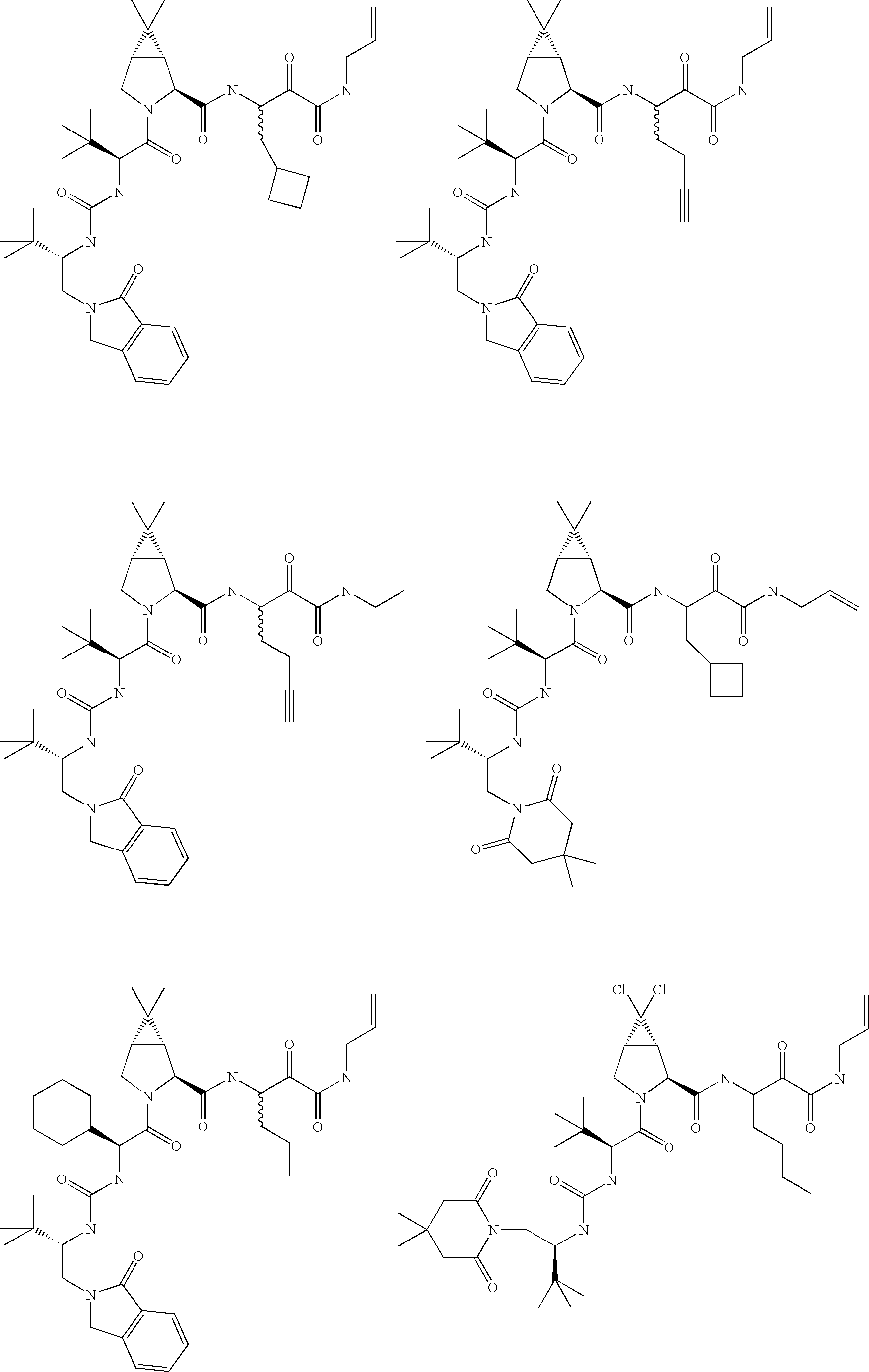 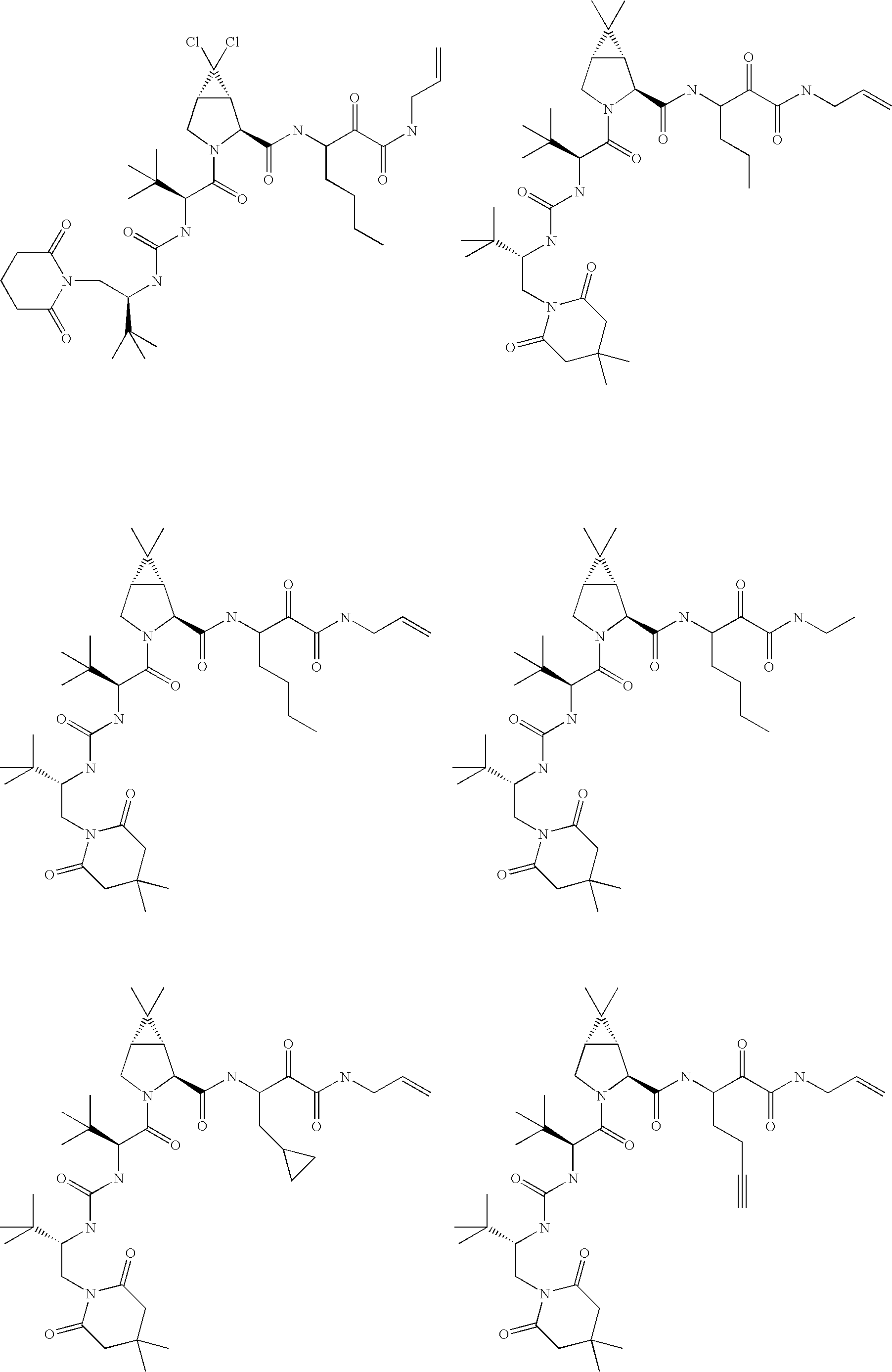 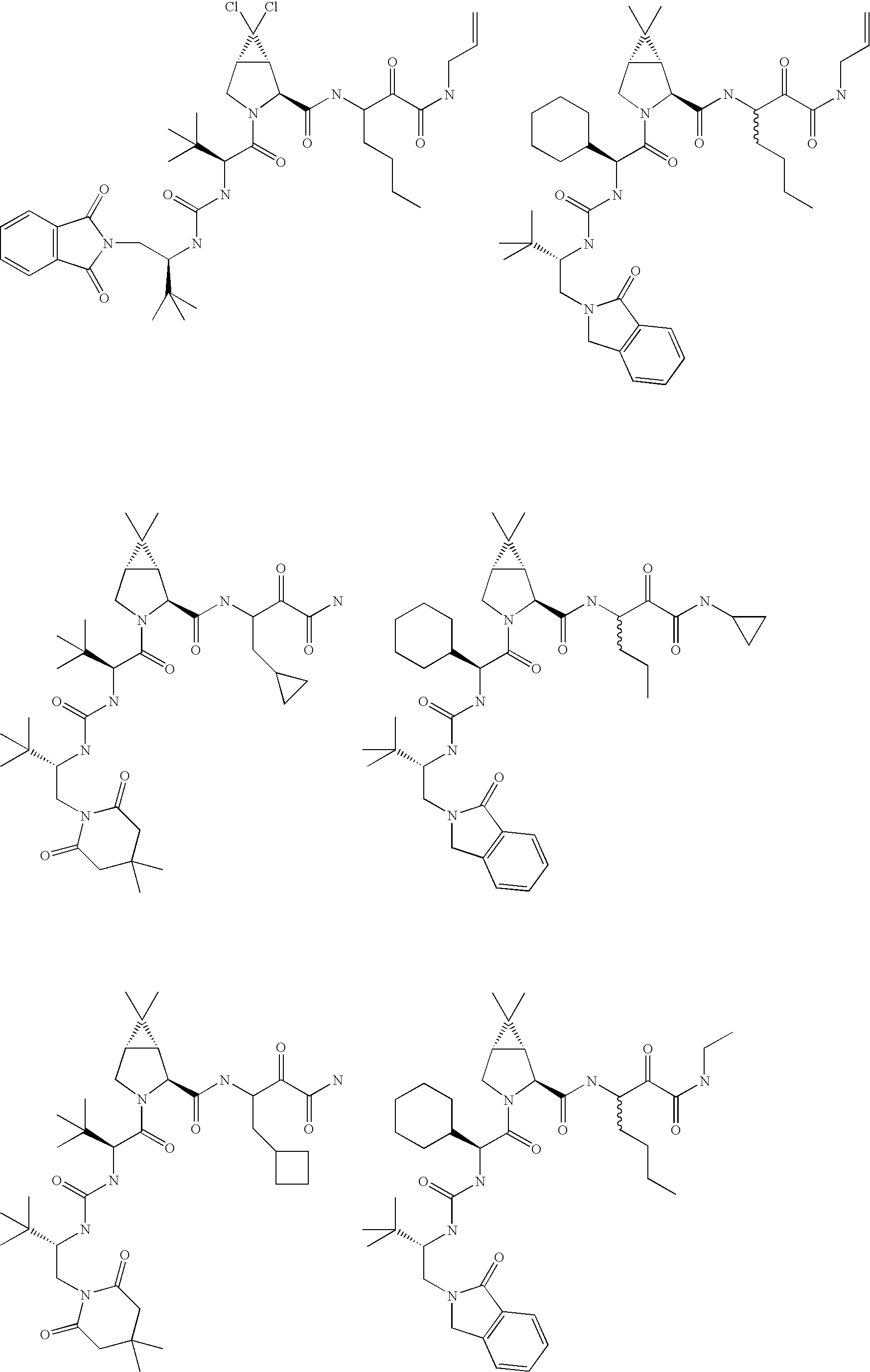 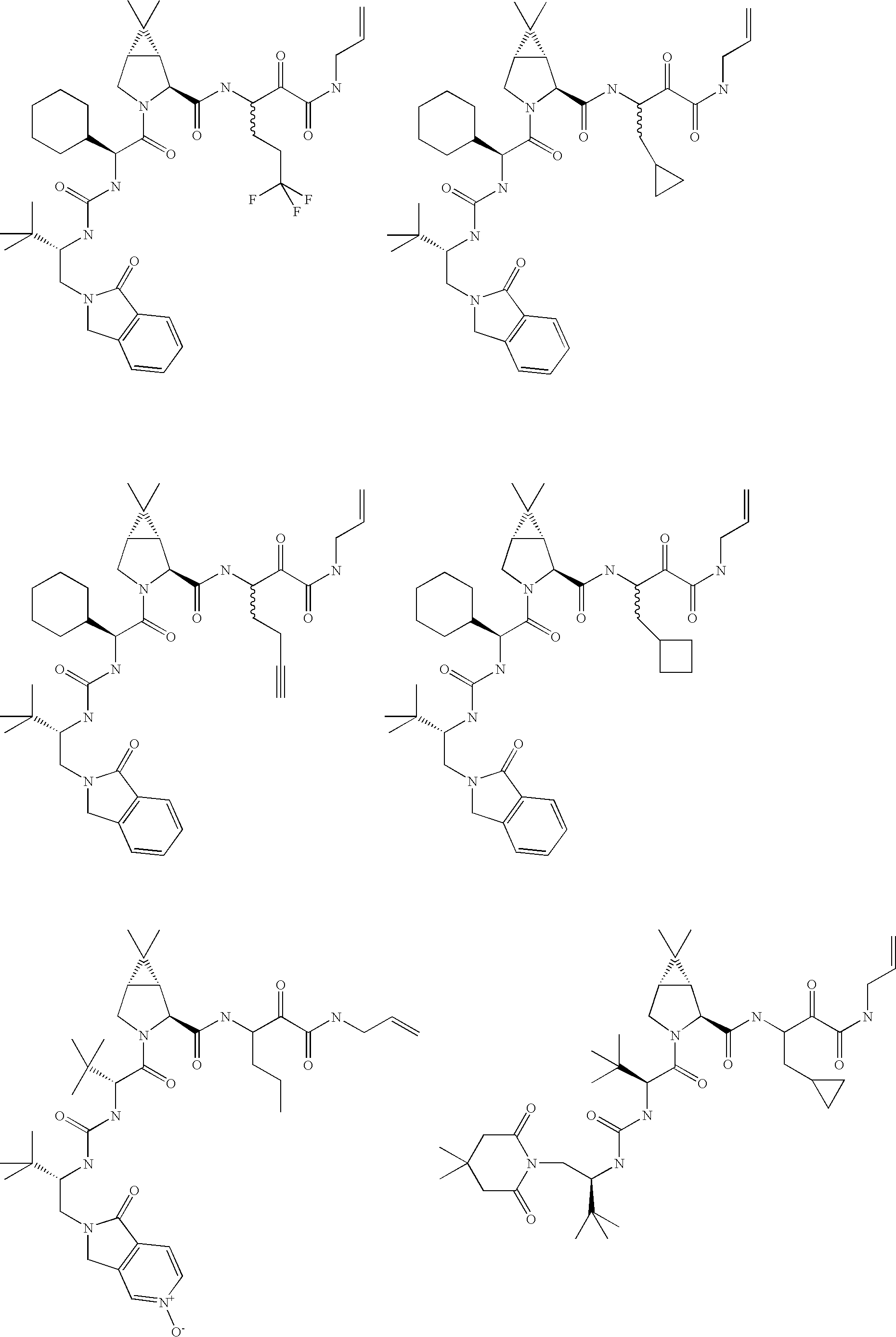 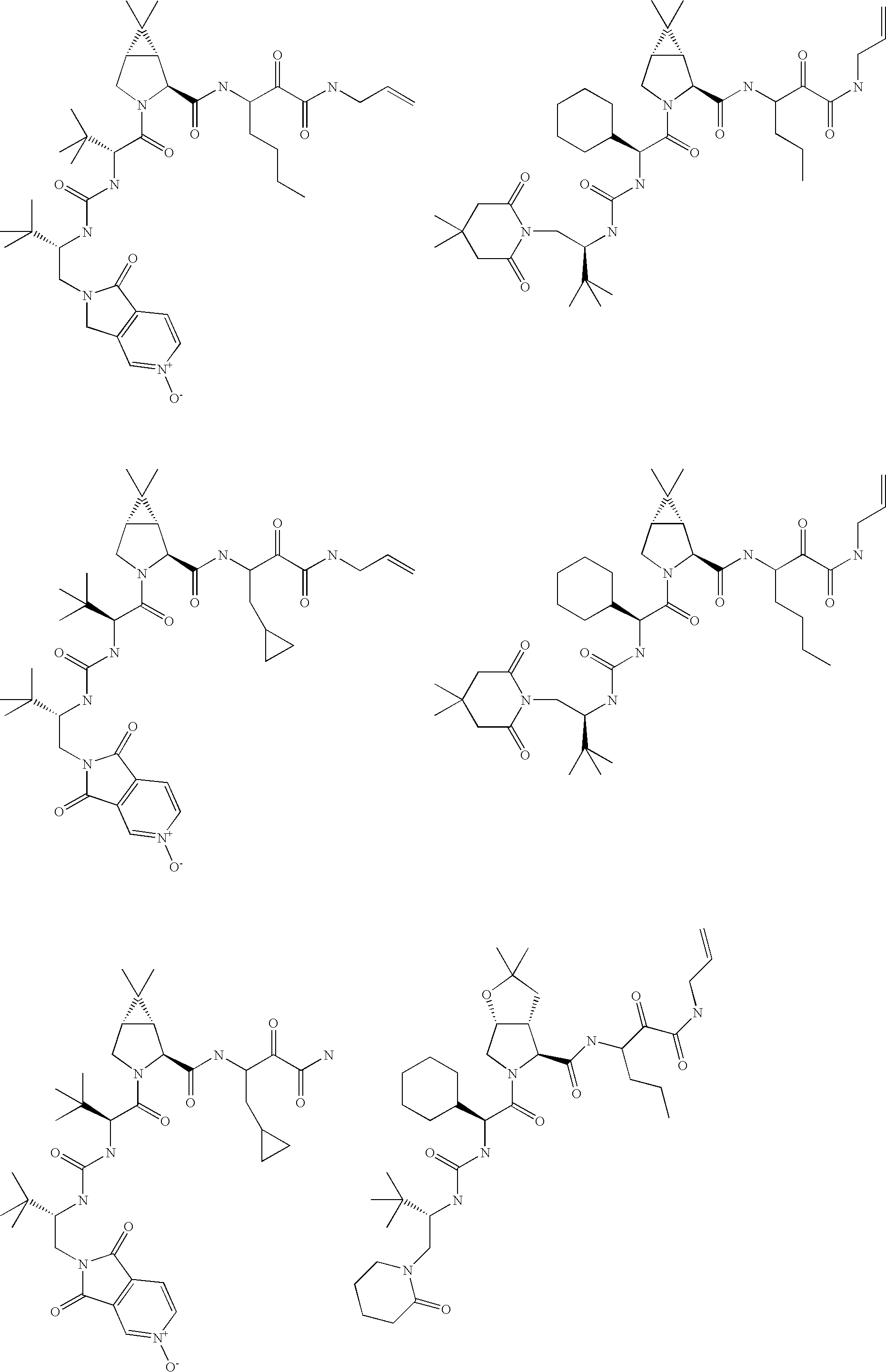 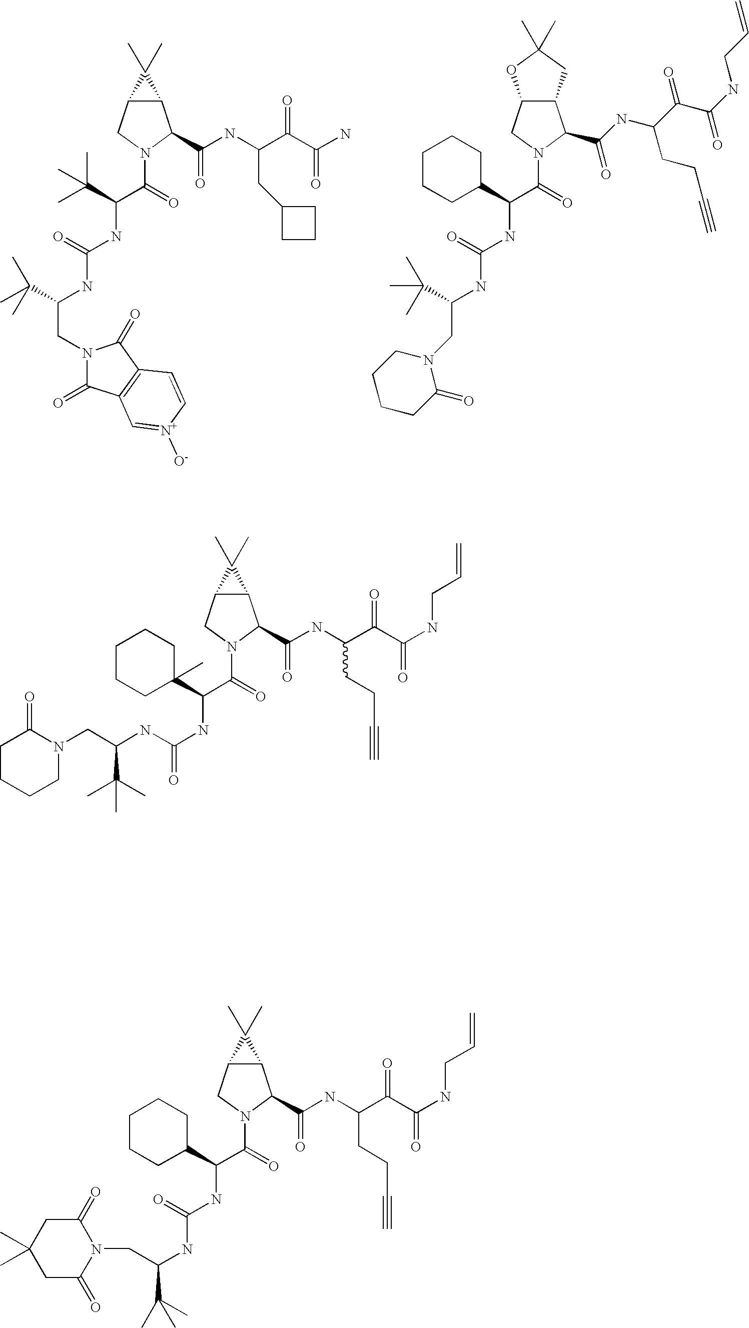 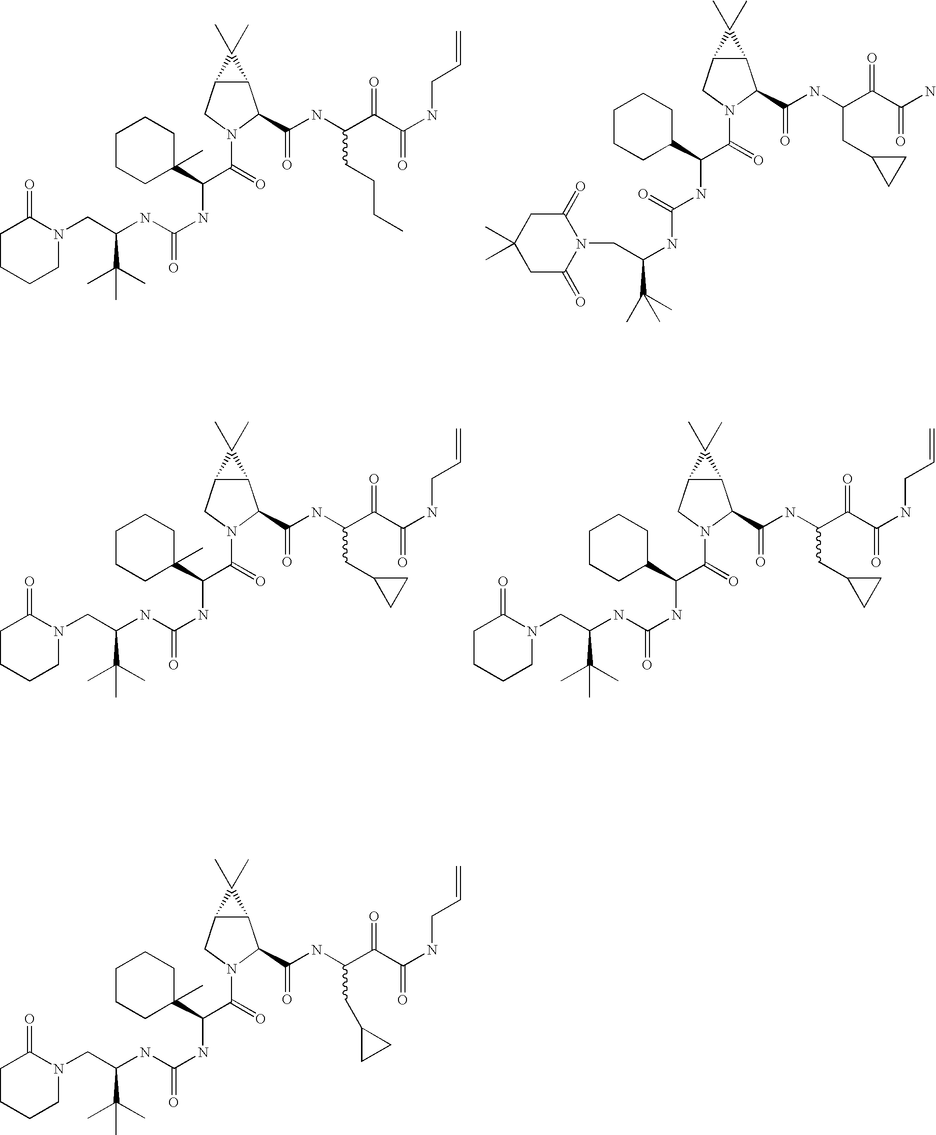 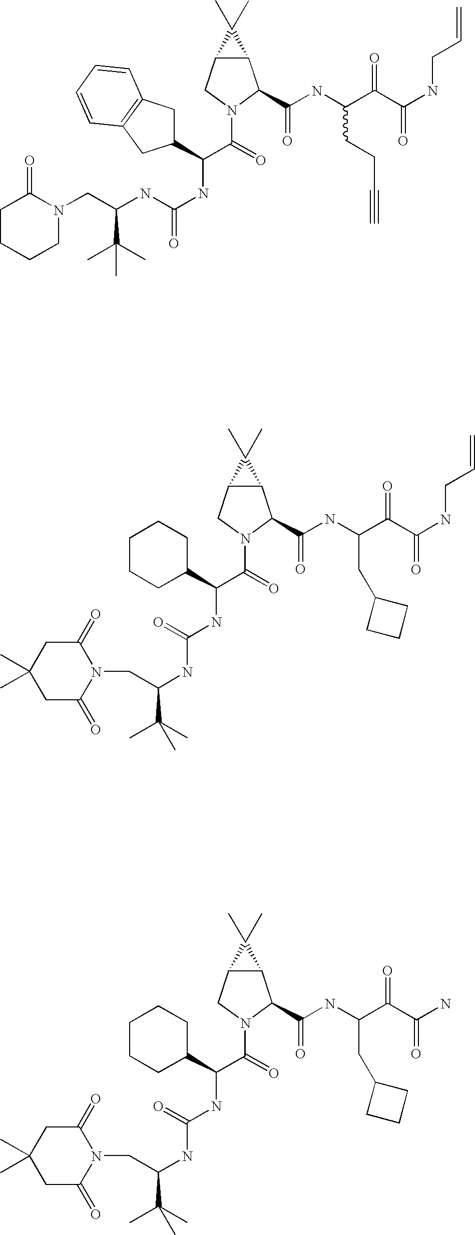 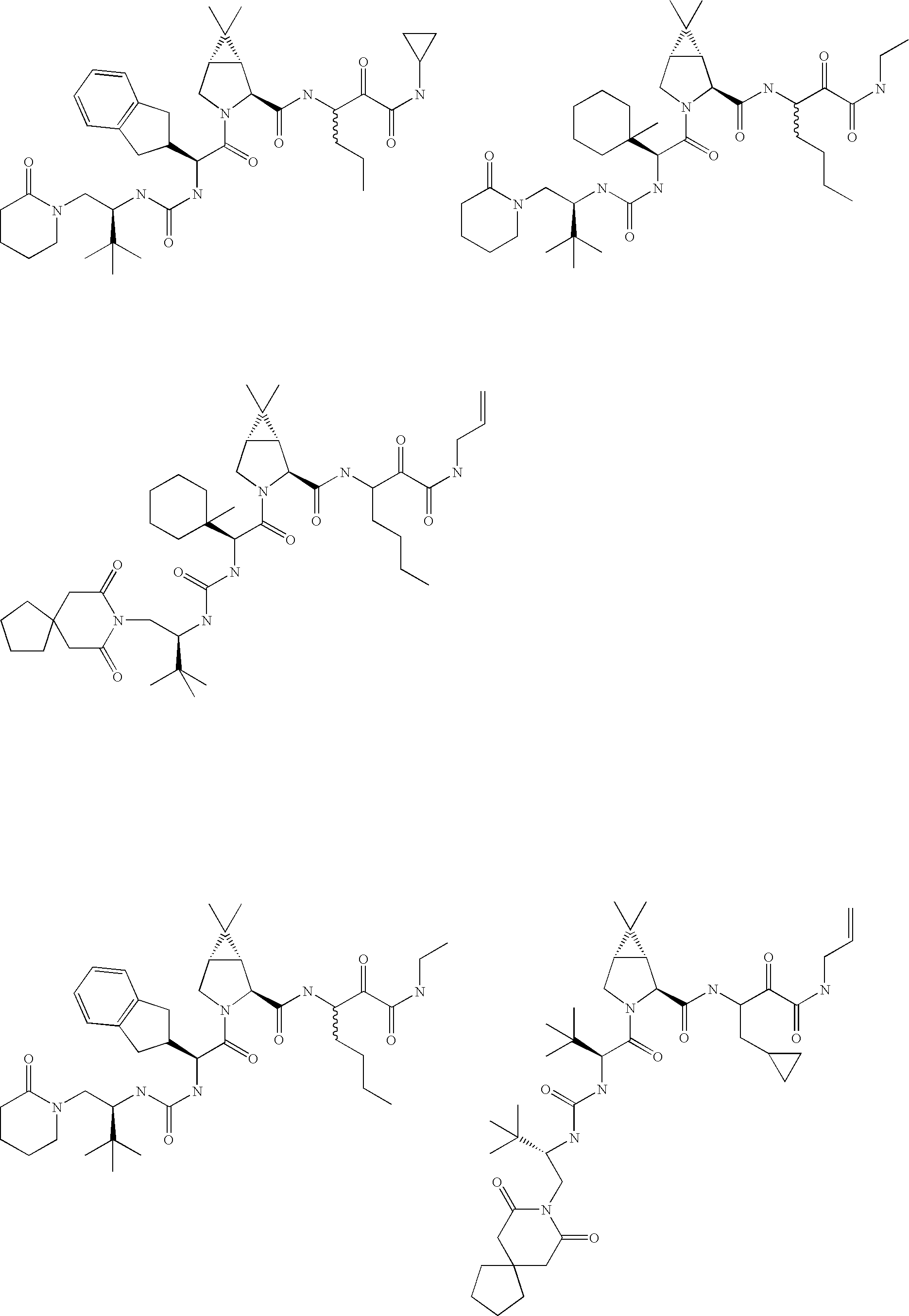 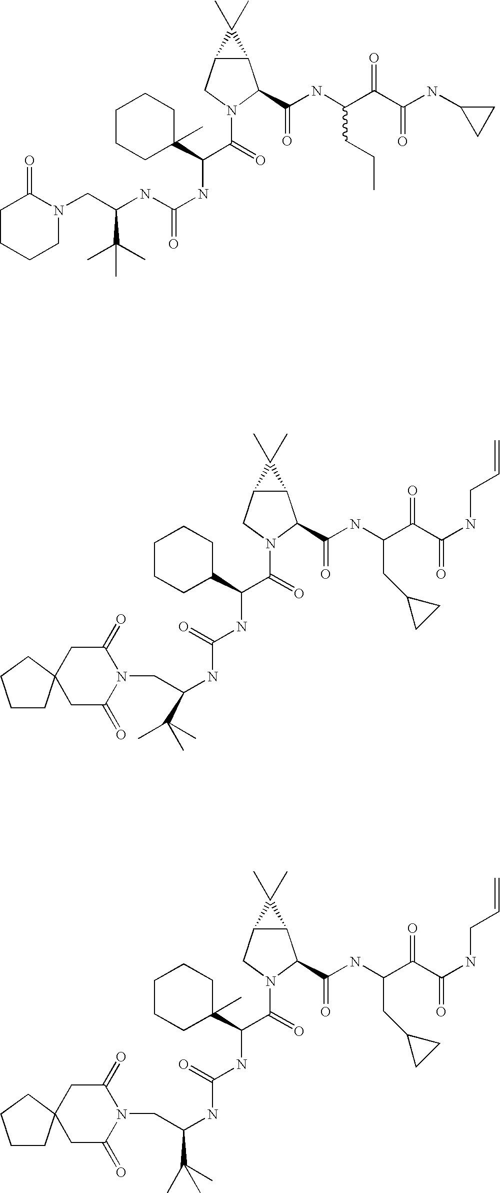 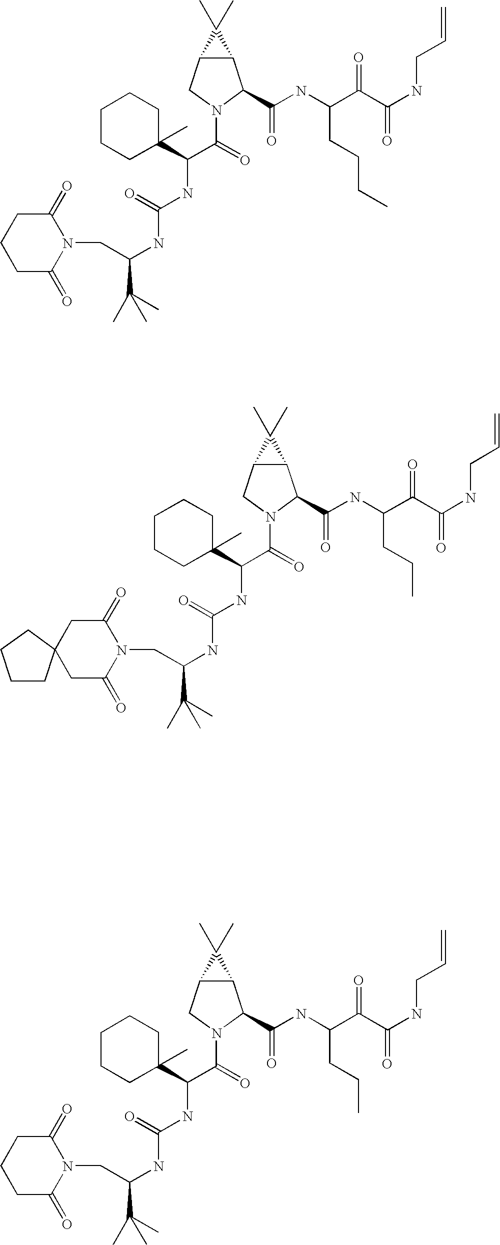 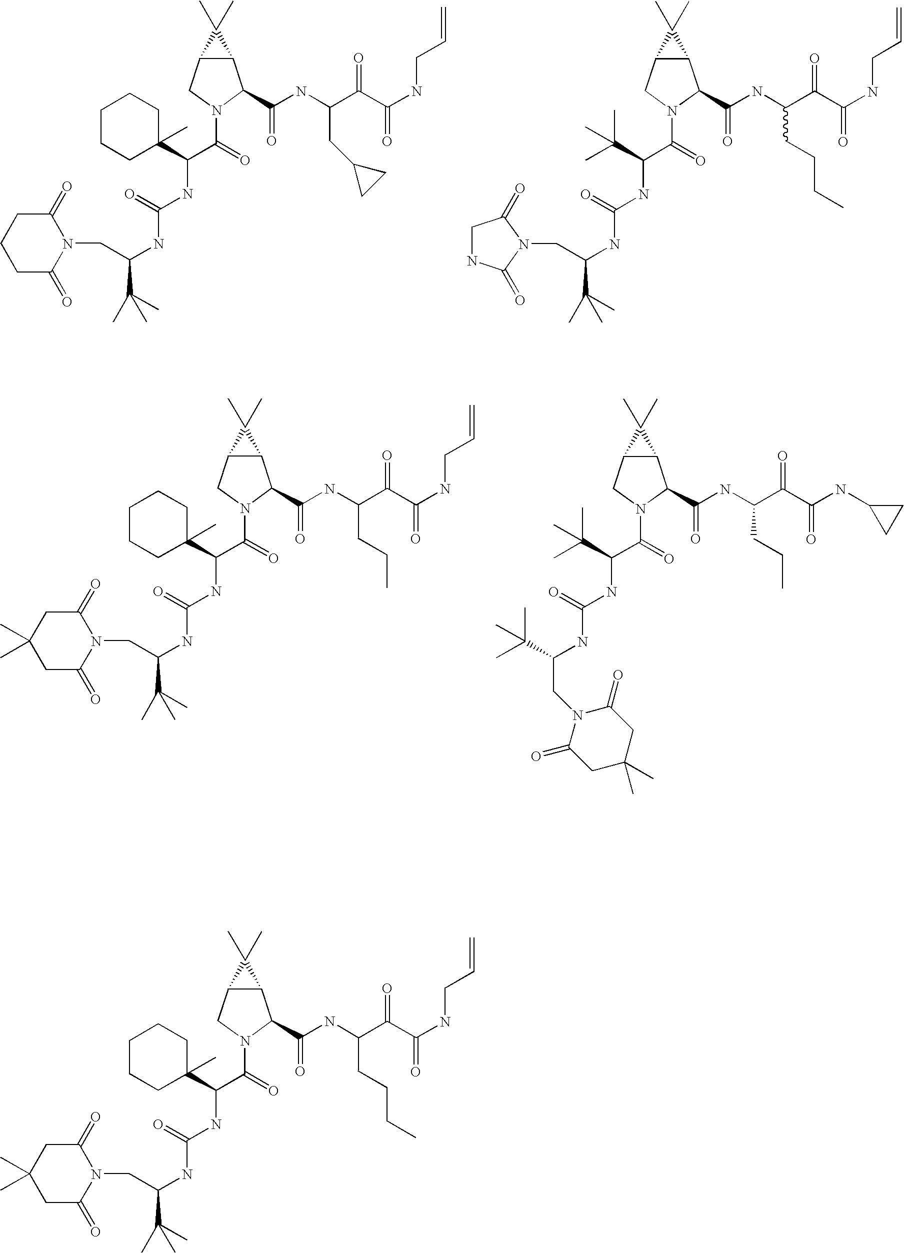 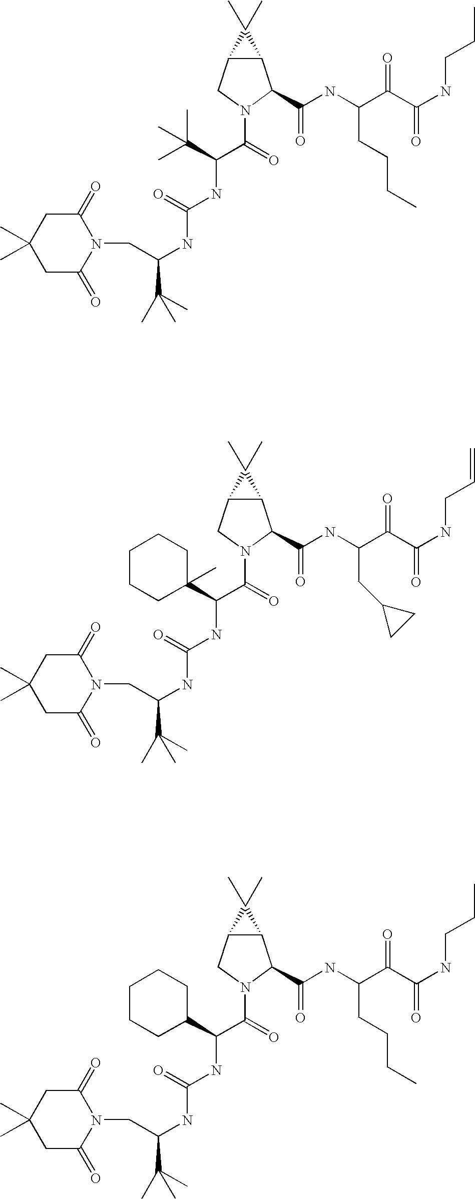 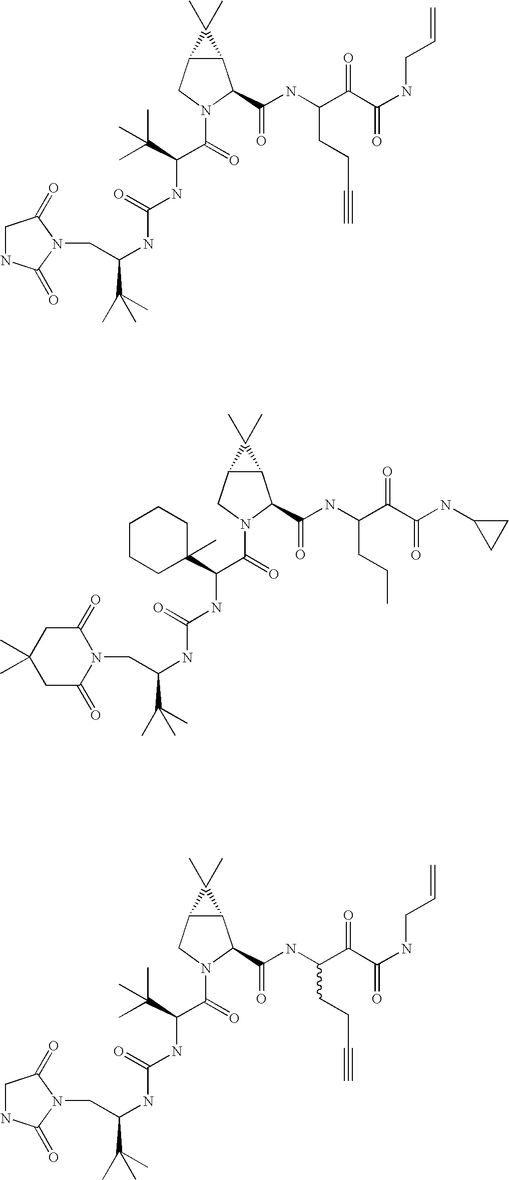 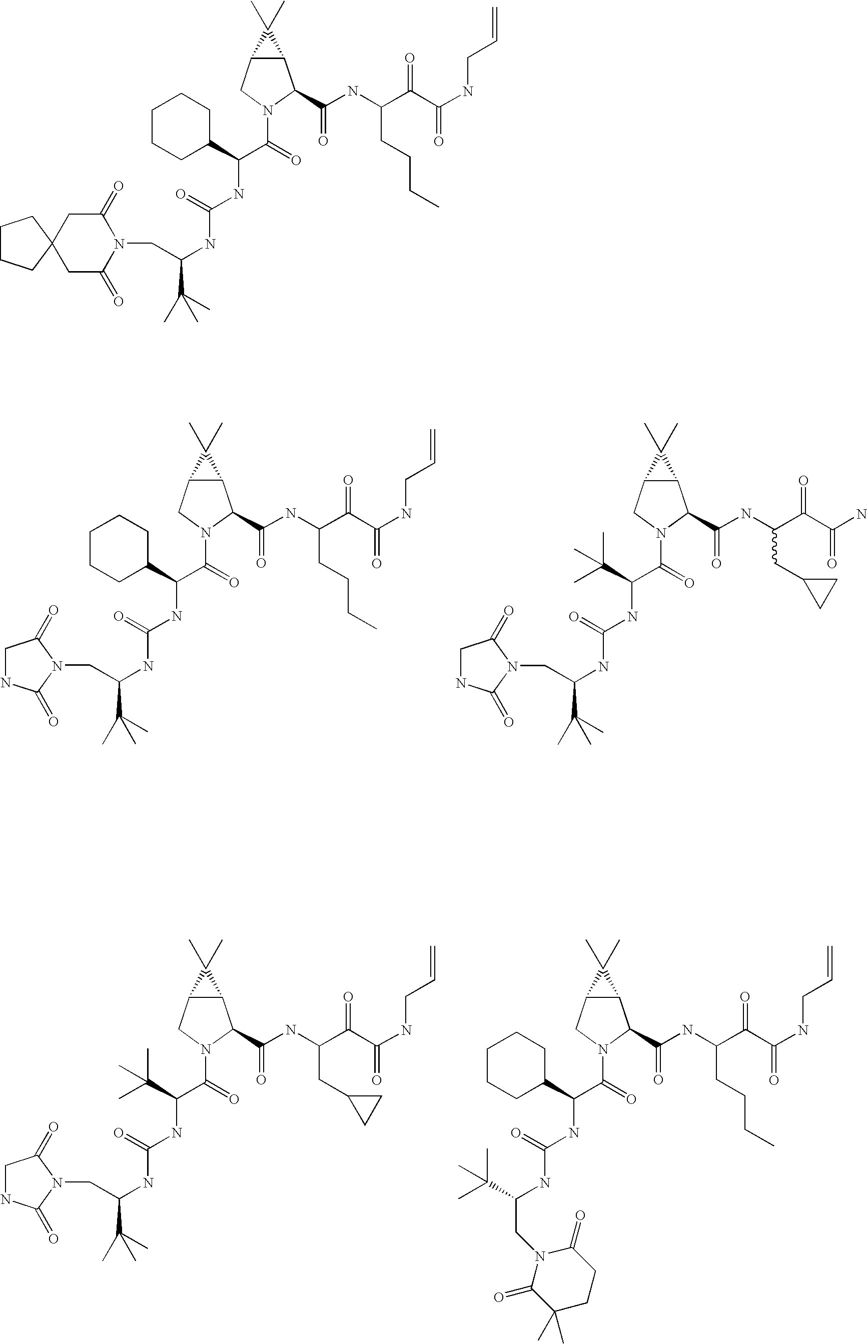 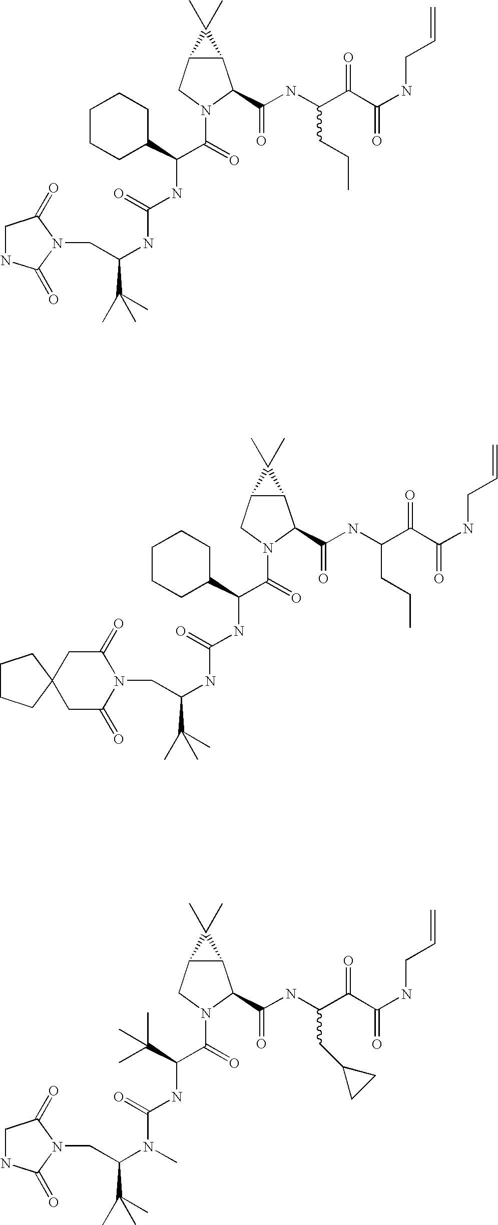
or a pharmaceutically acceptable salt, solvate or ester thereof.

Compounds of formula XIV are disclosed in U.S. patent application Ser. No. 11/064,673 filed Feb. 24, 2005. The preparation of these compounds is disclosed in the experimental section of this application set forth hereinbelow.

Non-limiting examples of certain compounds disclosed in U.S. patent application Ser. No. 11/064,673 are: 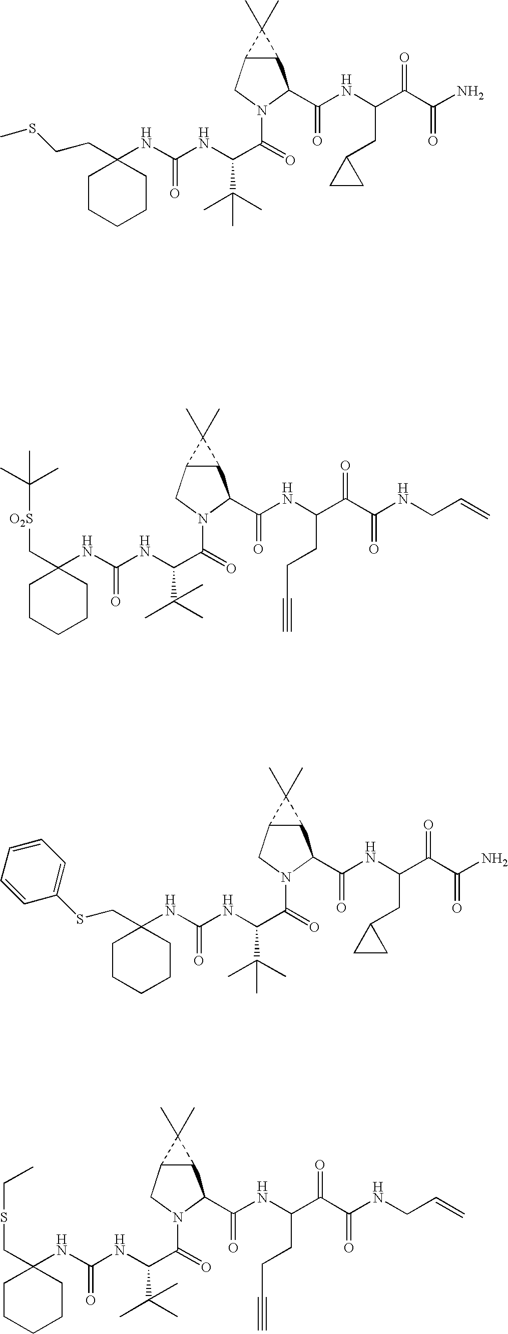 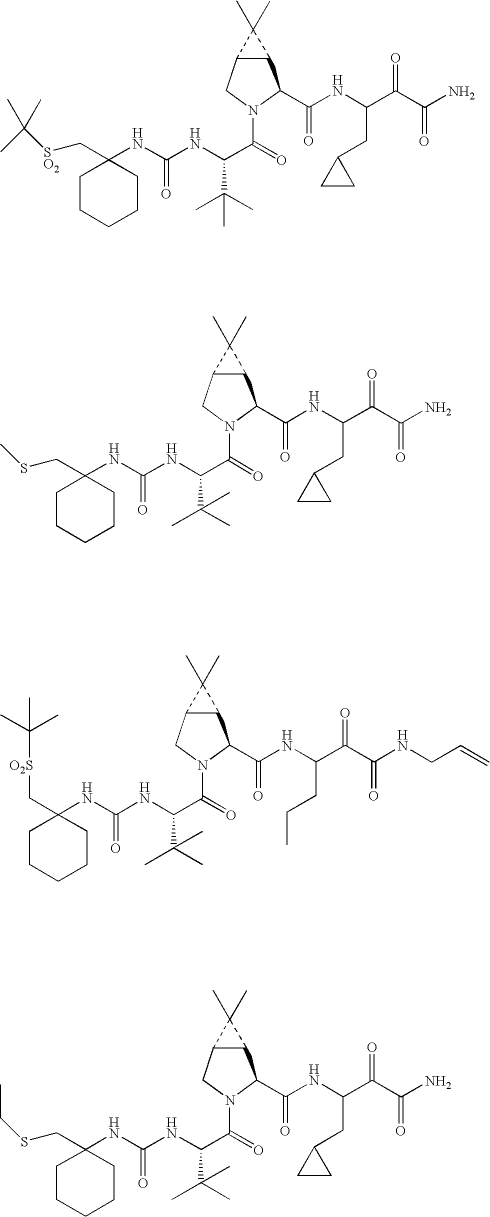 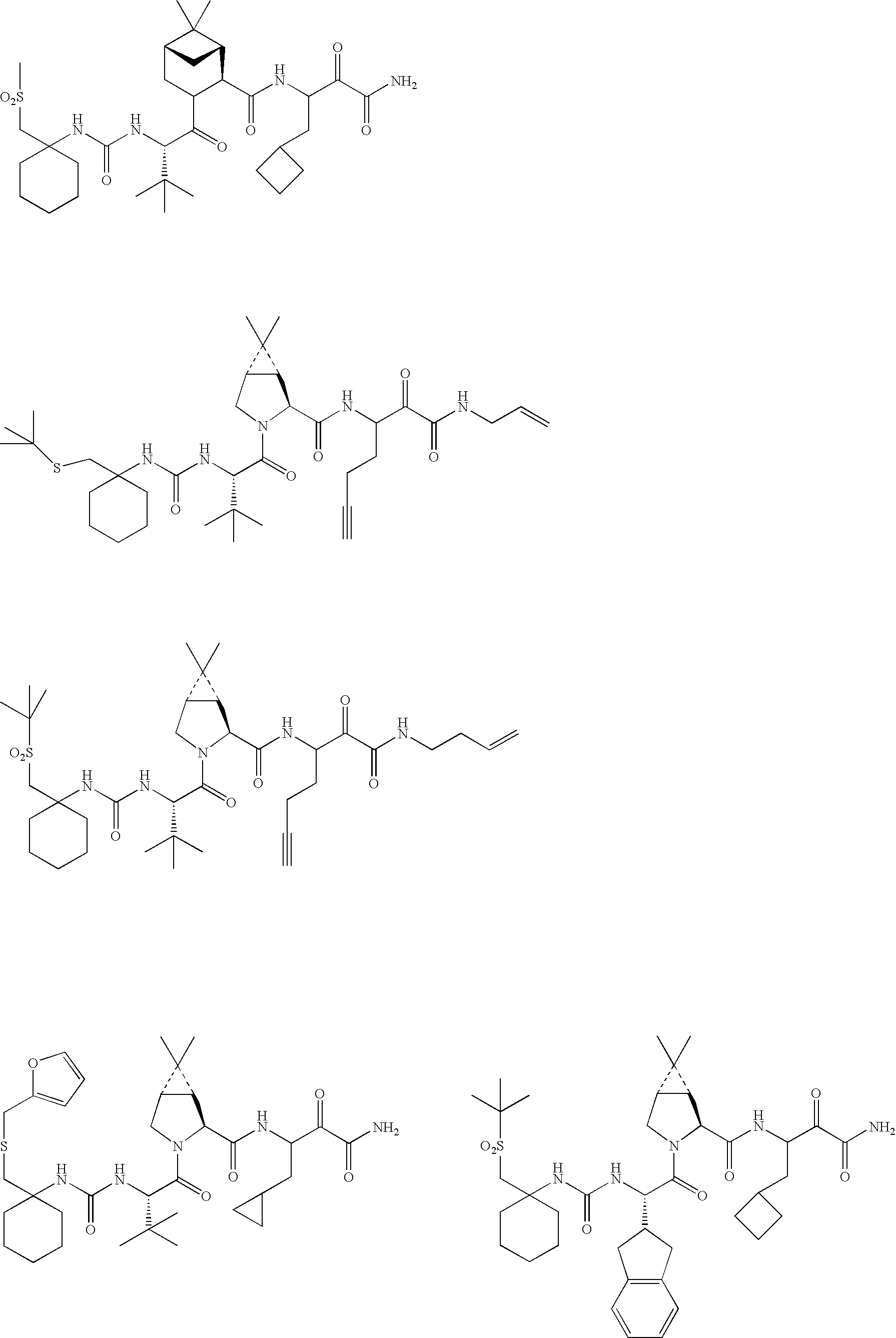 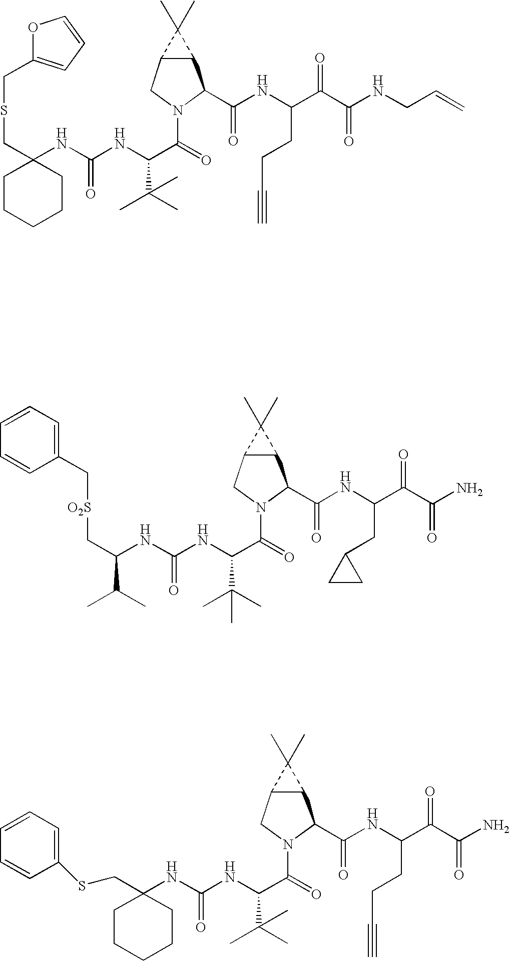 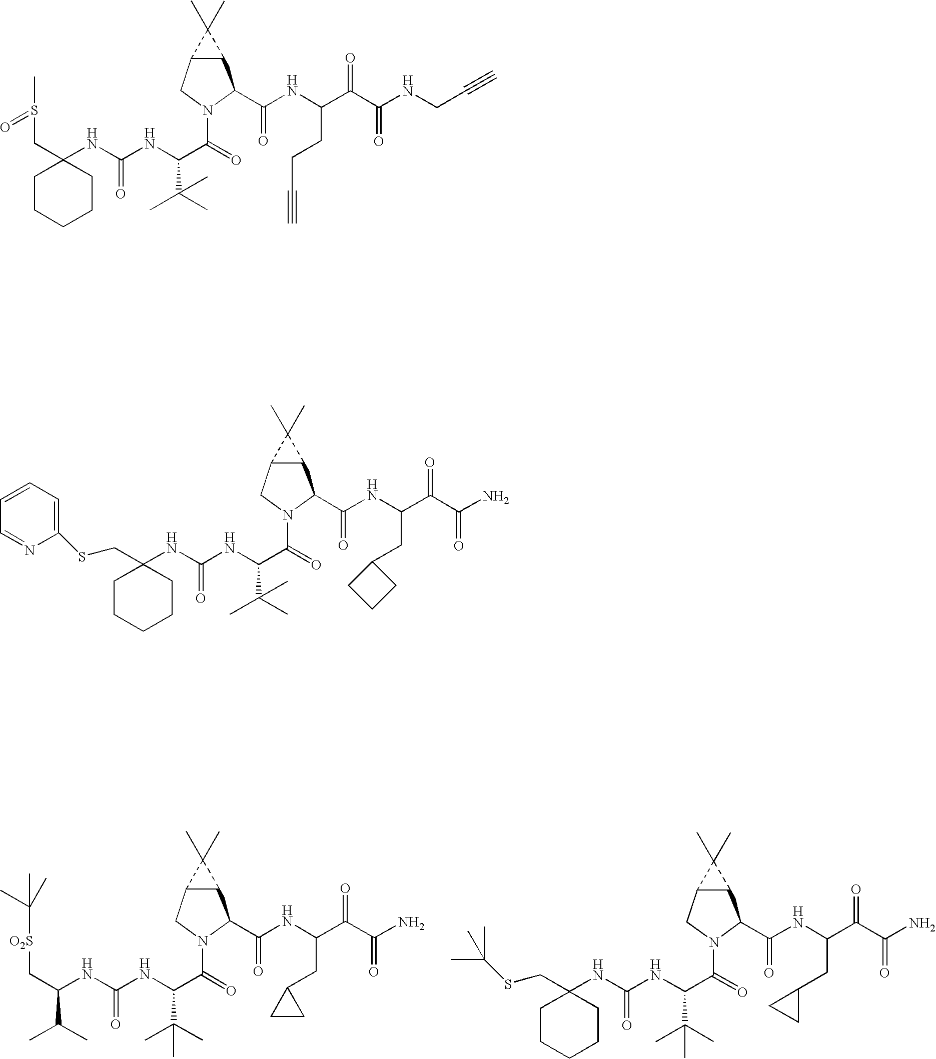 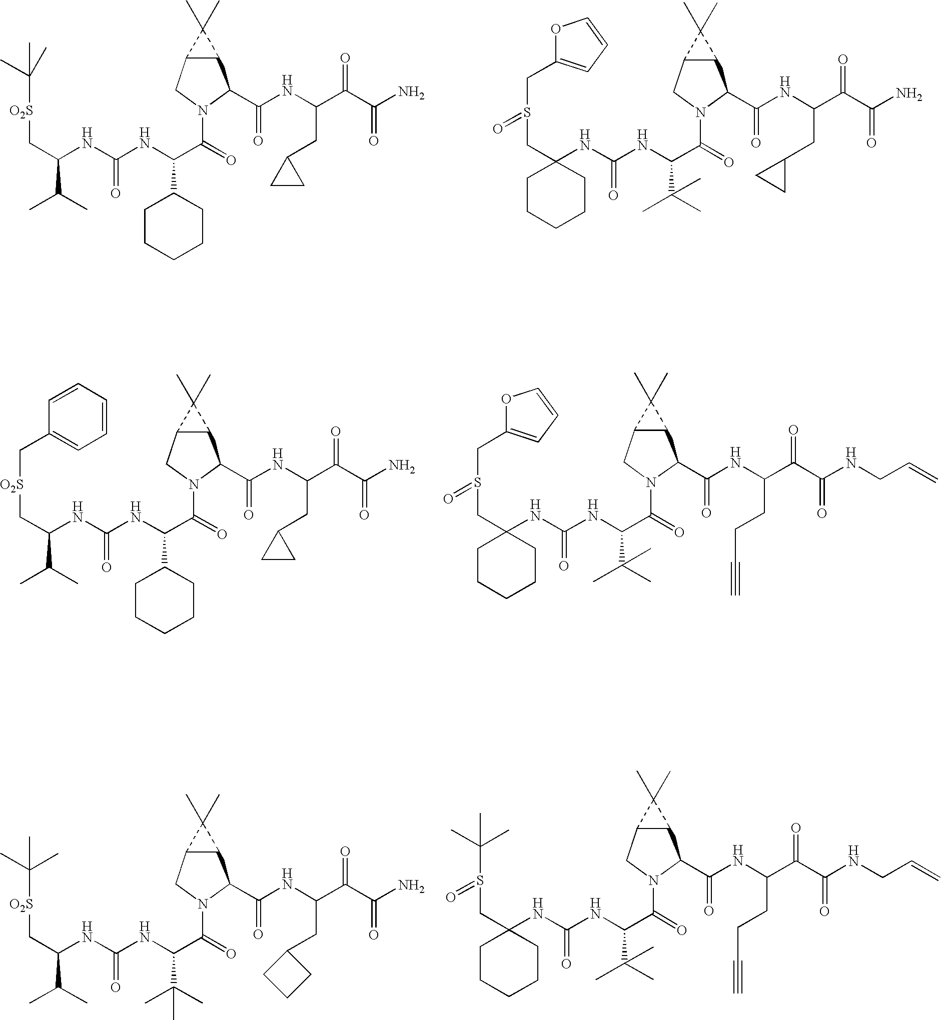 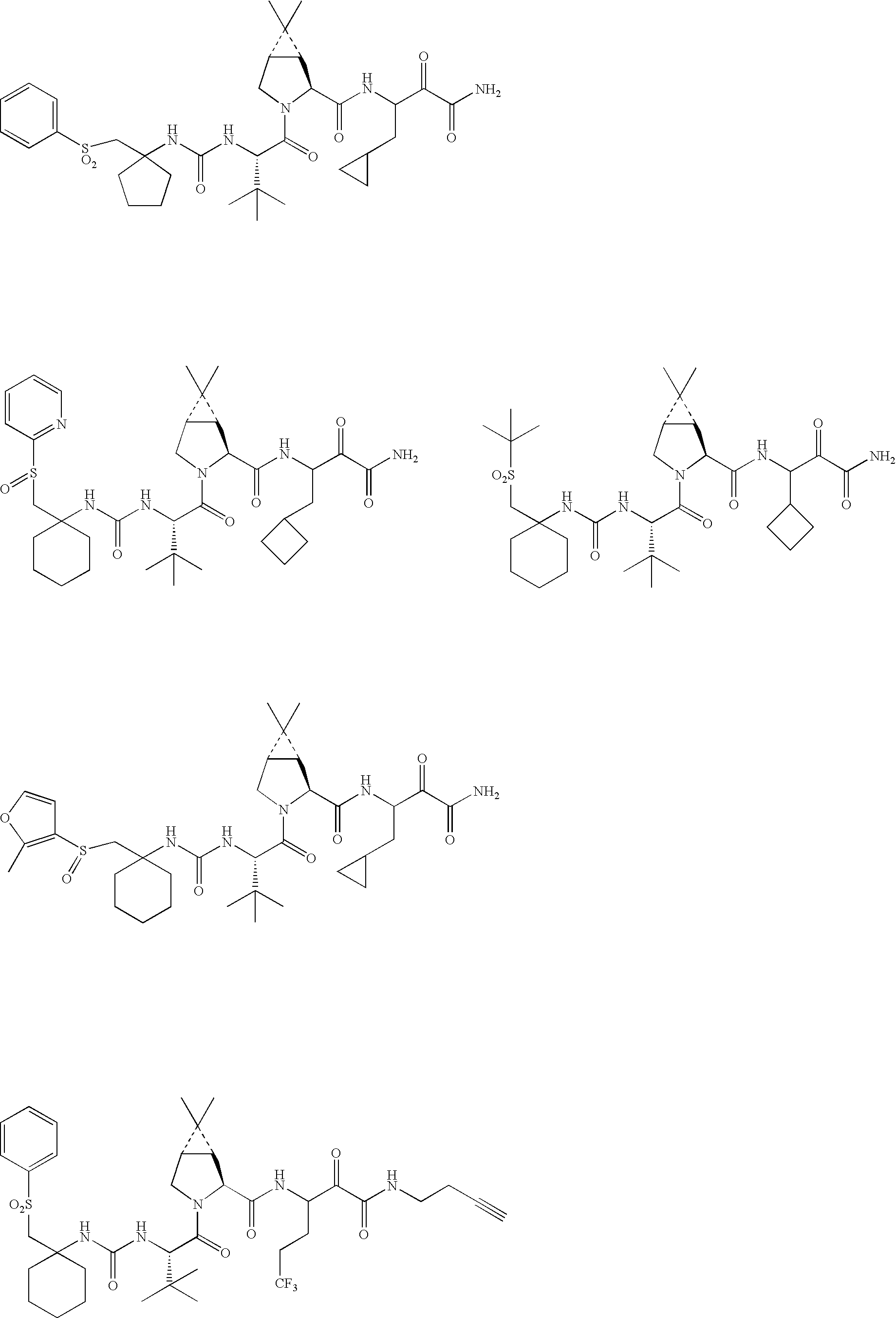 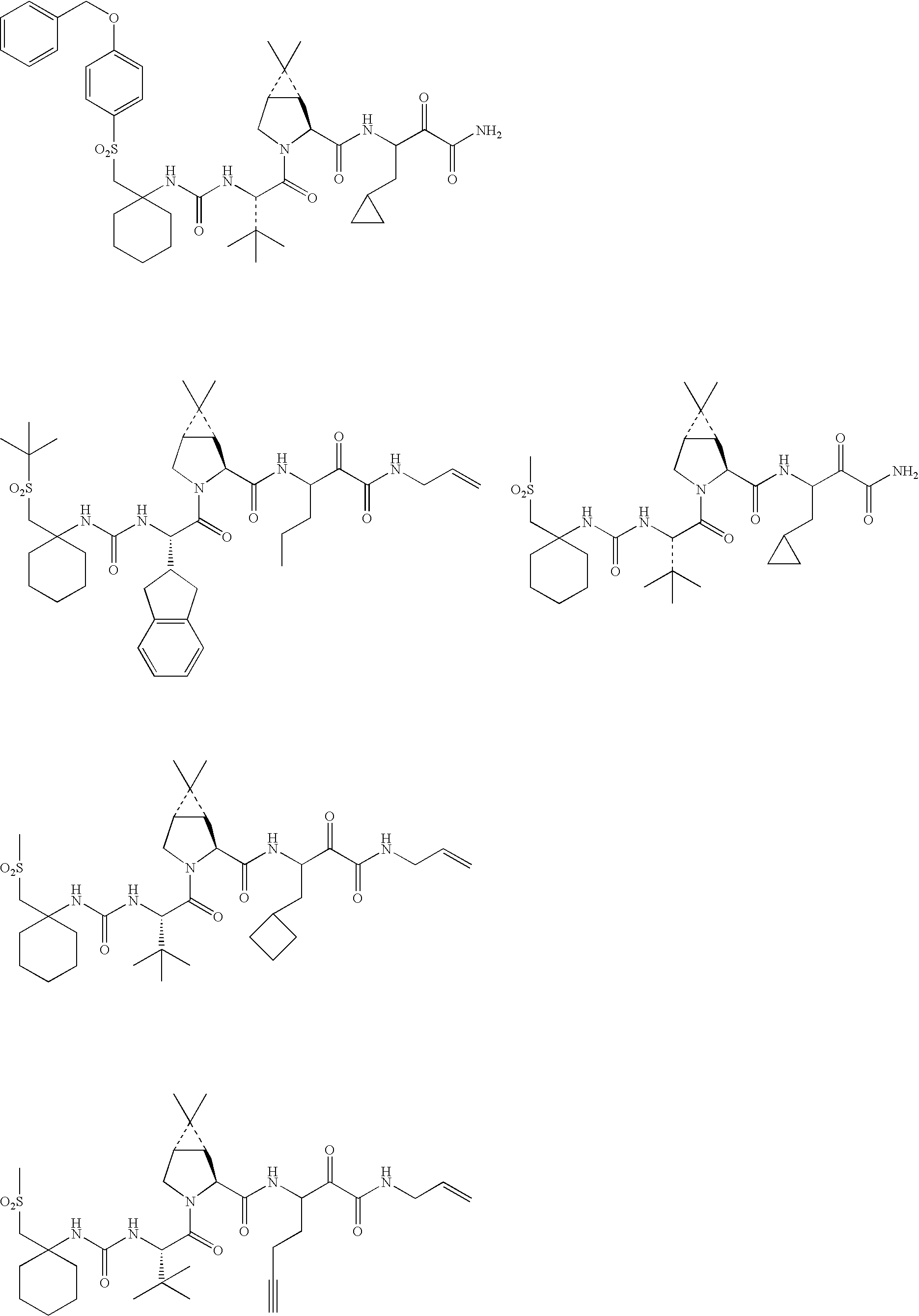 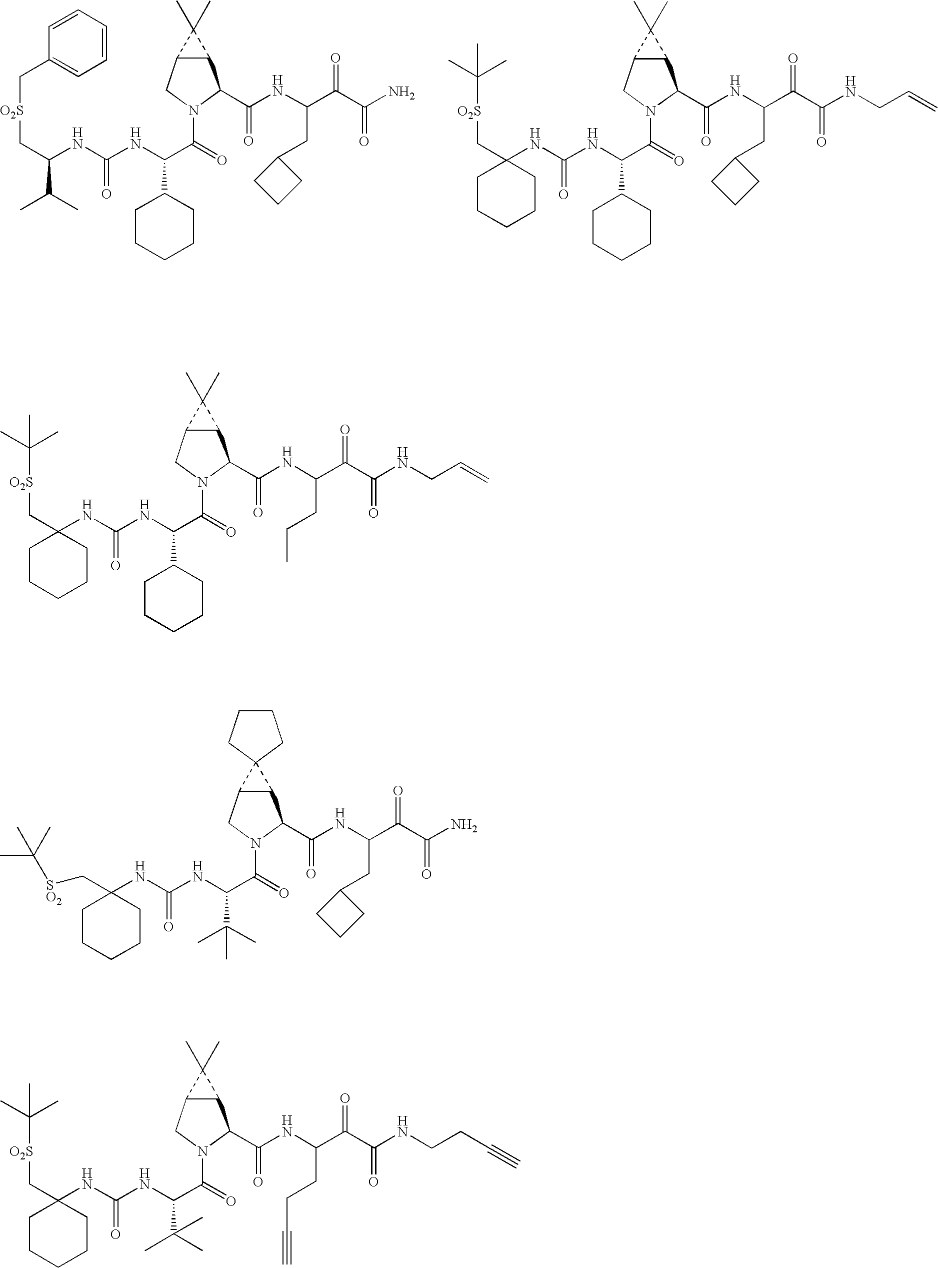 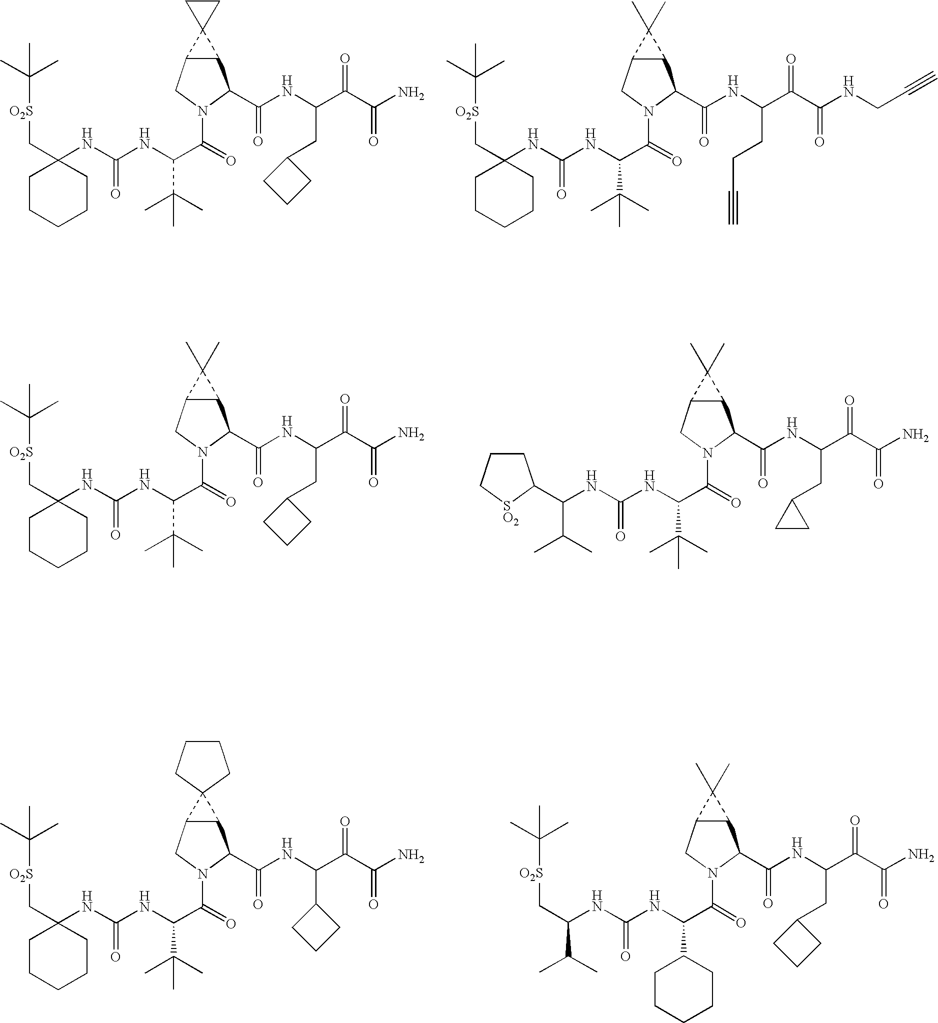 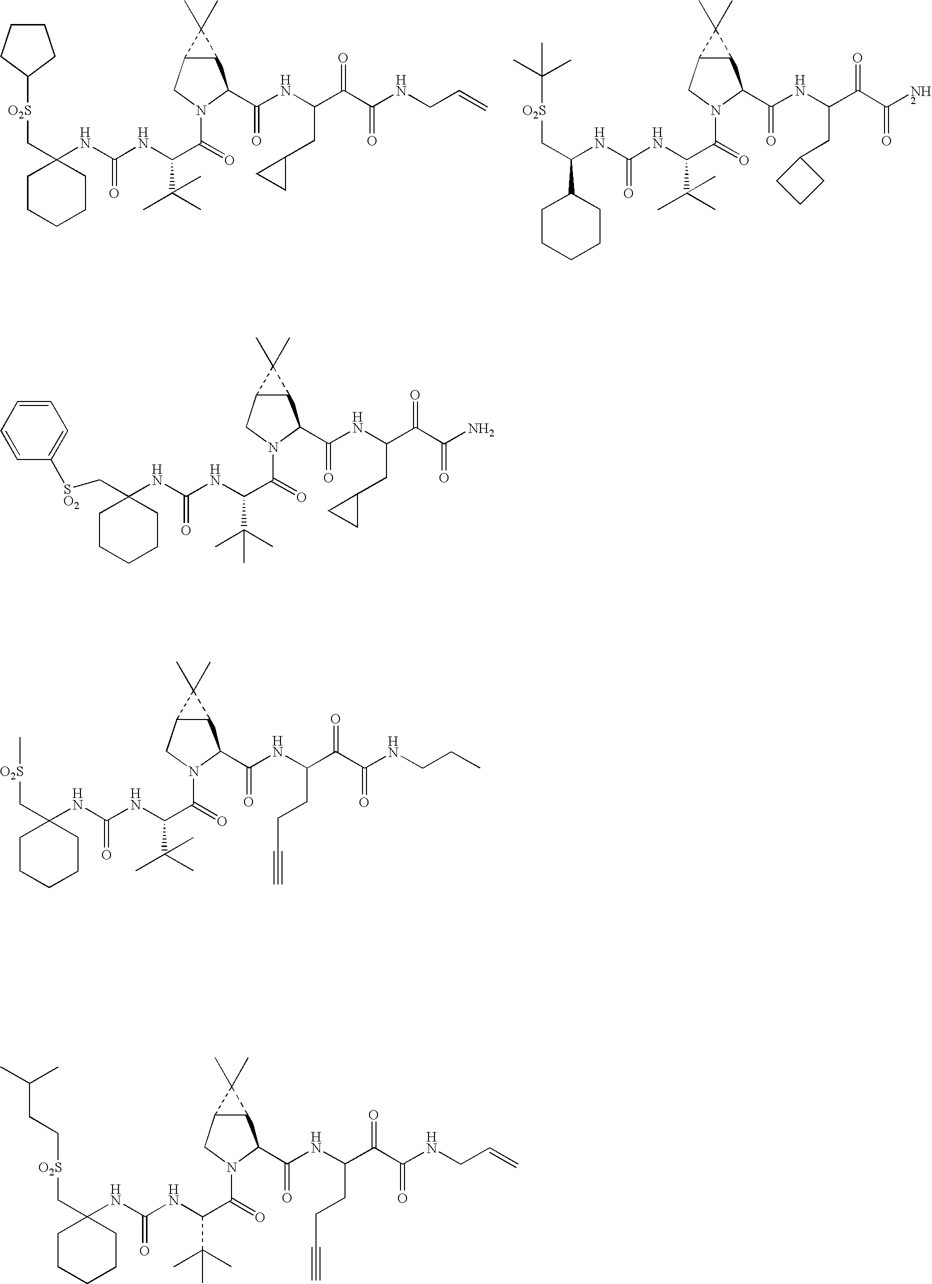 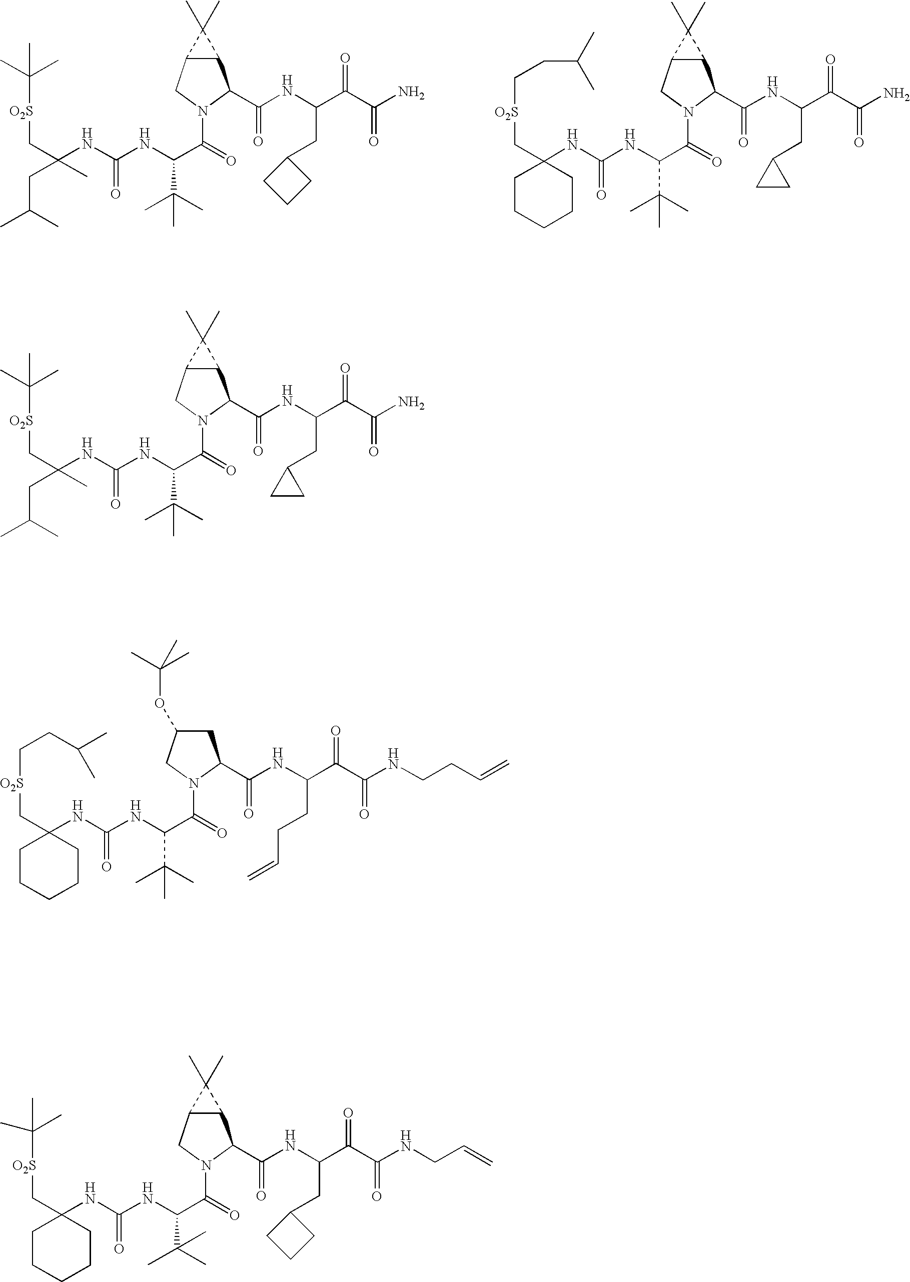 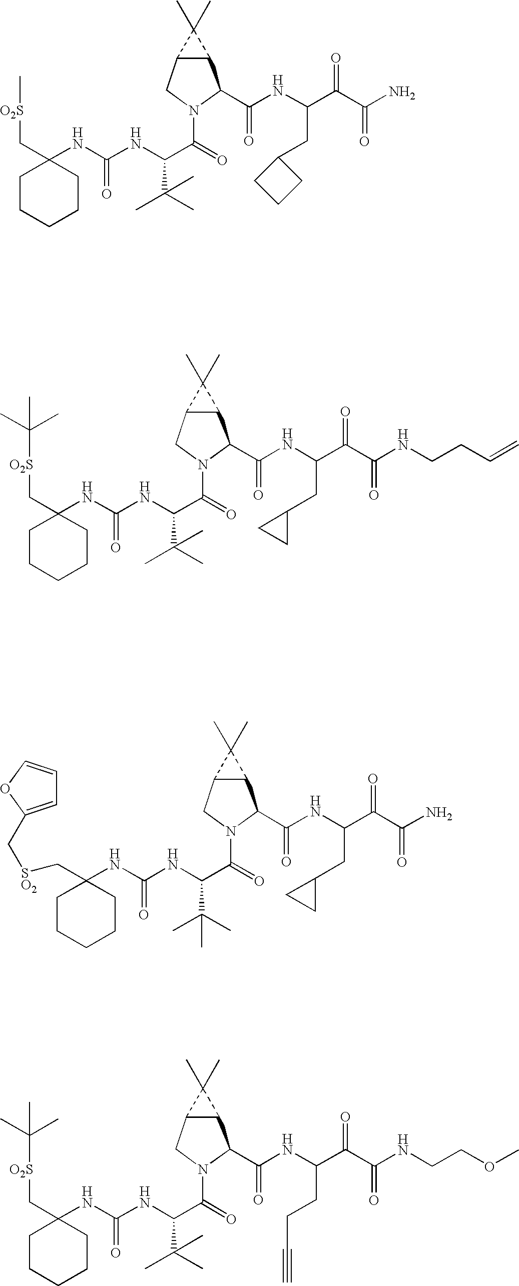 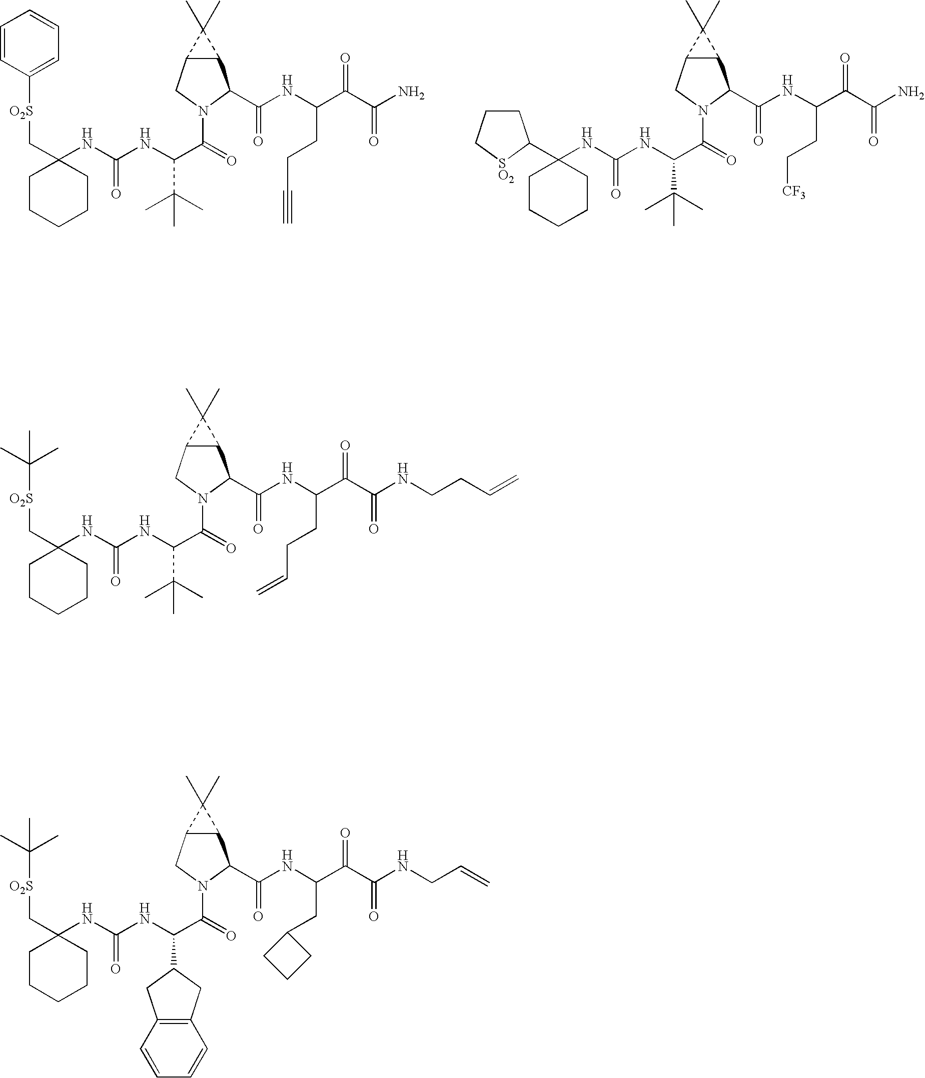 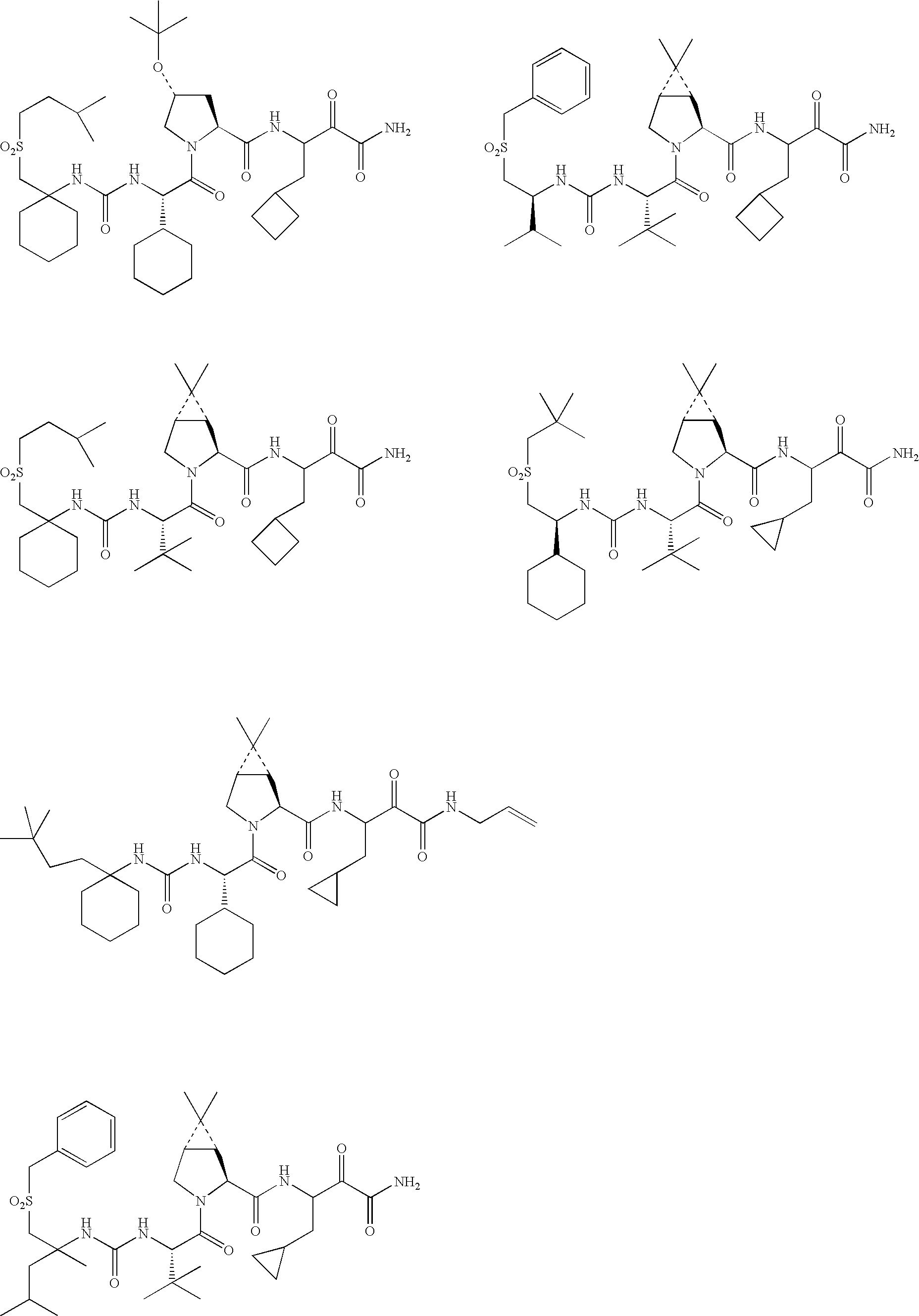 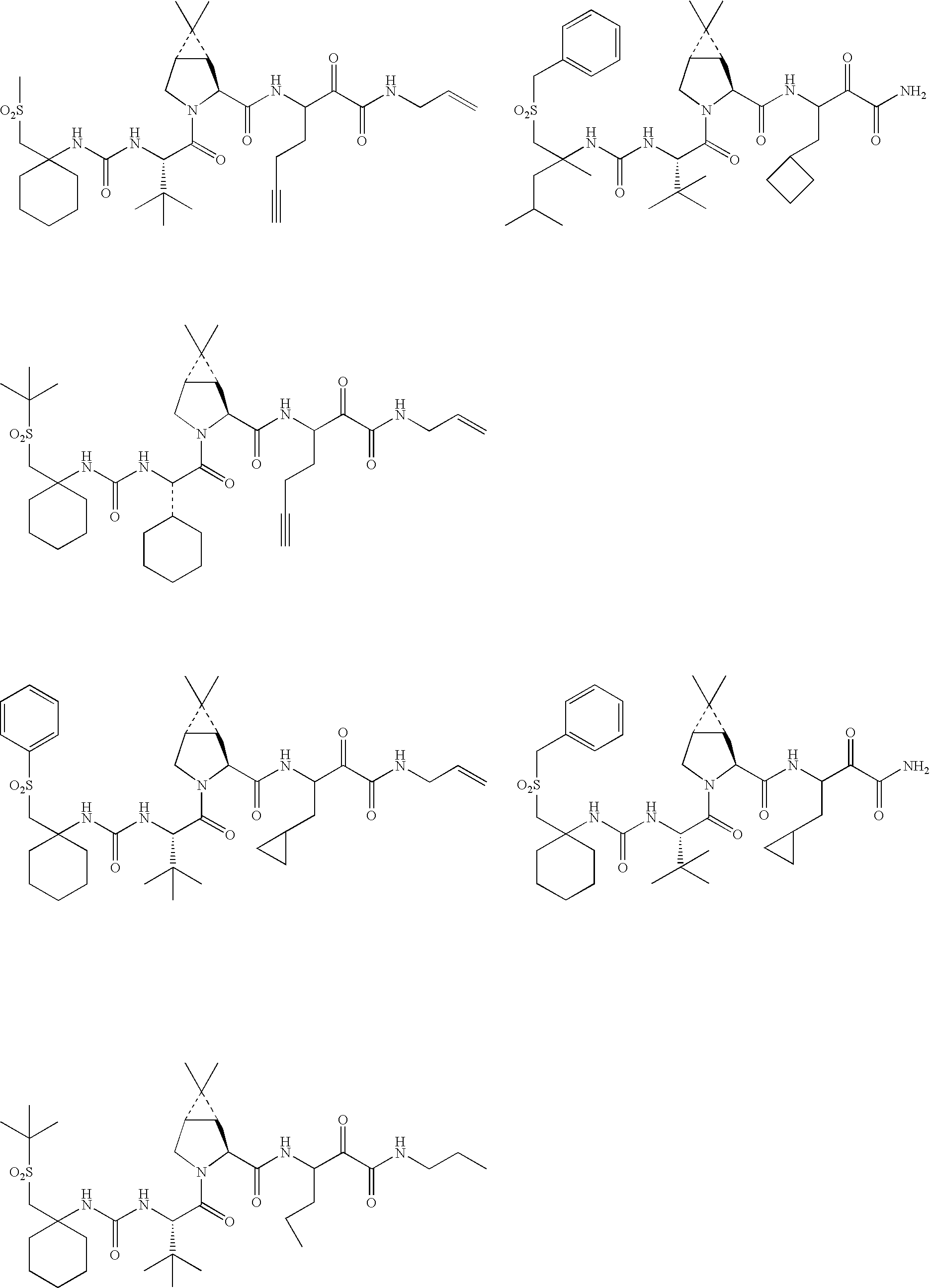 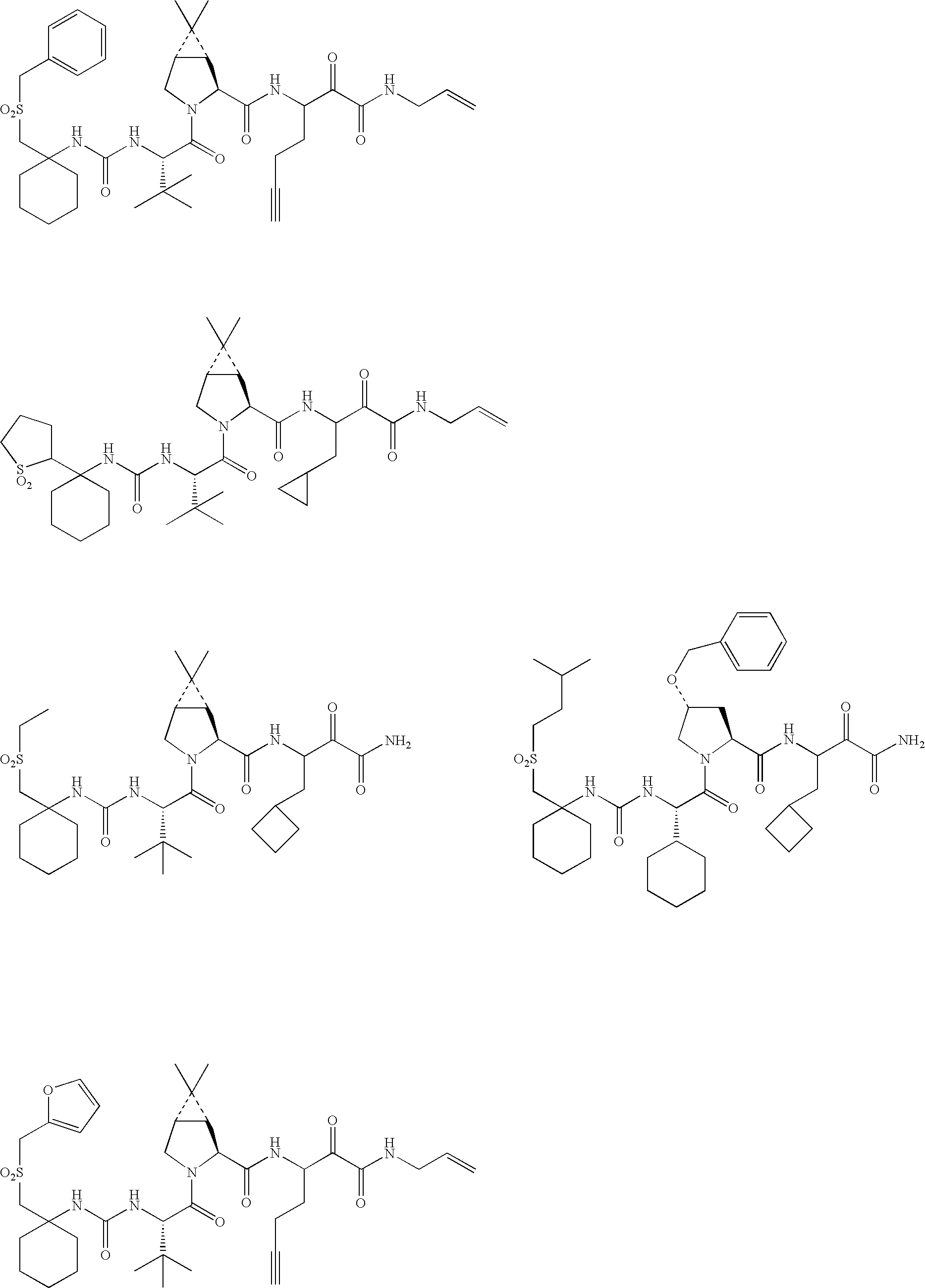 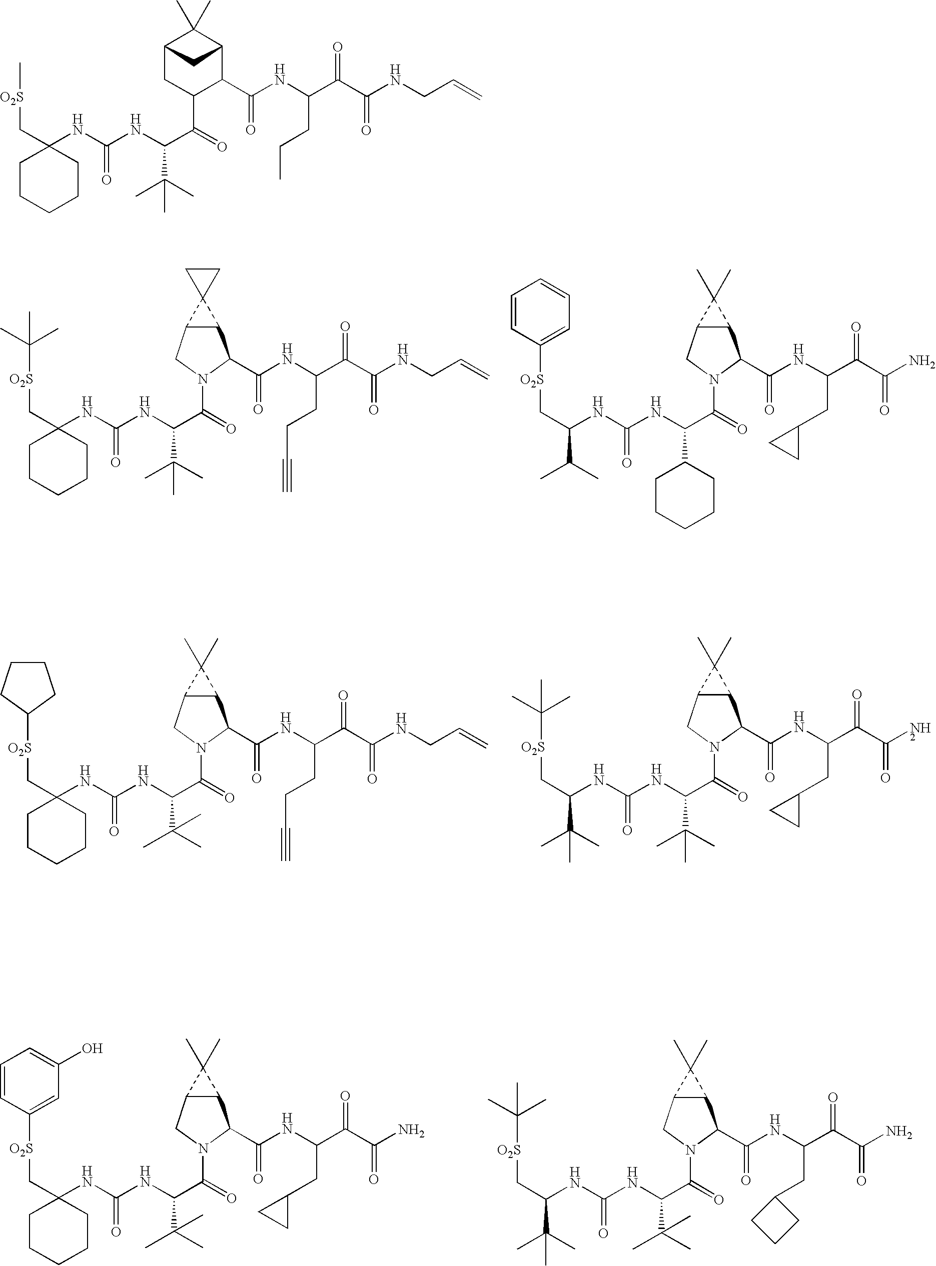 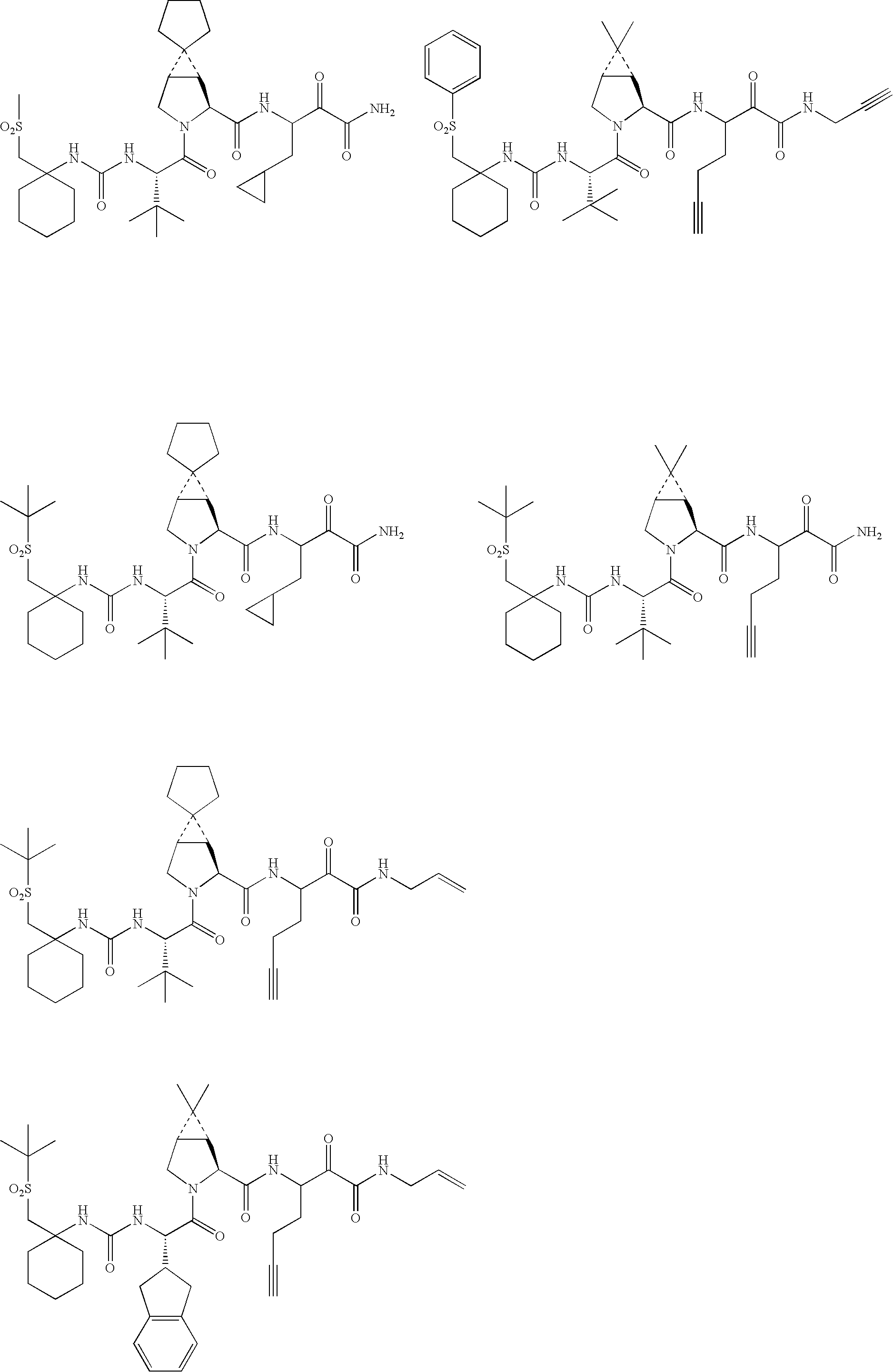 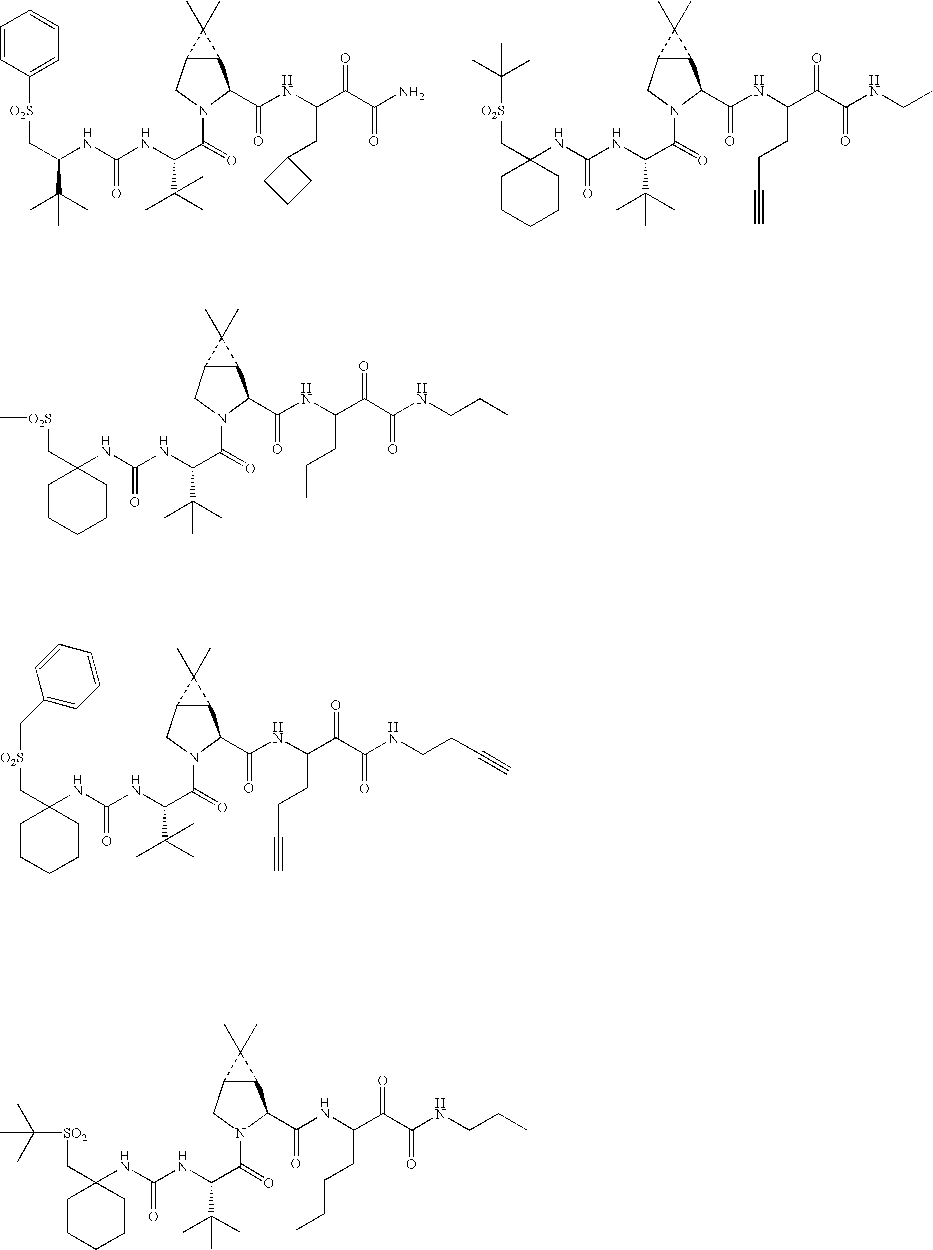 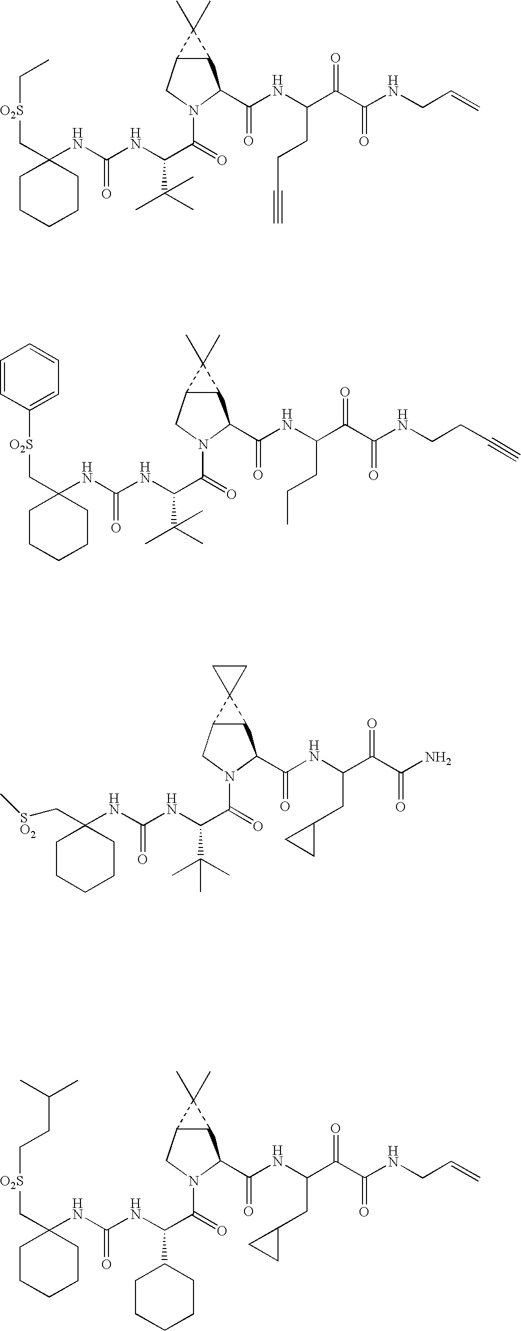 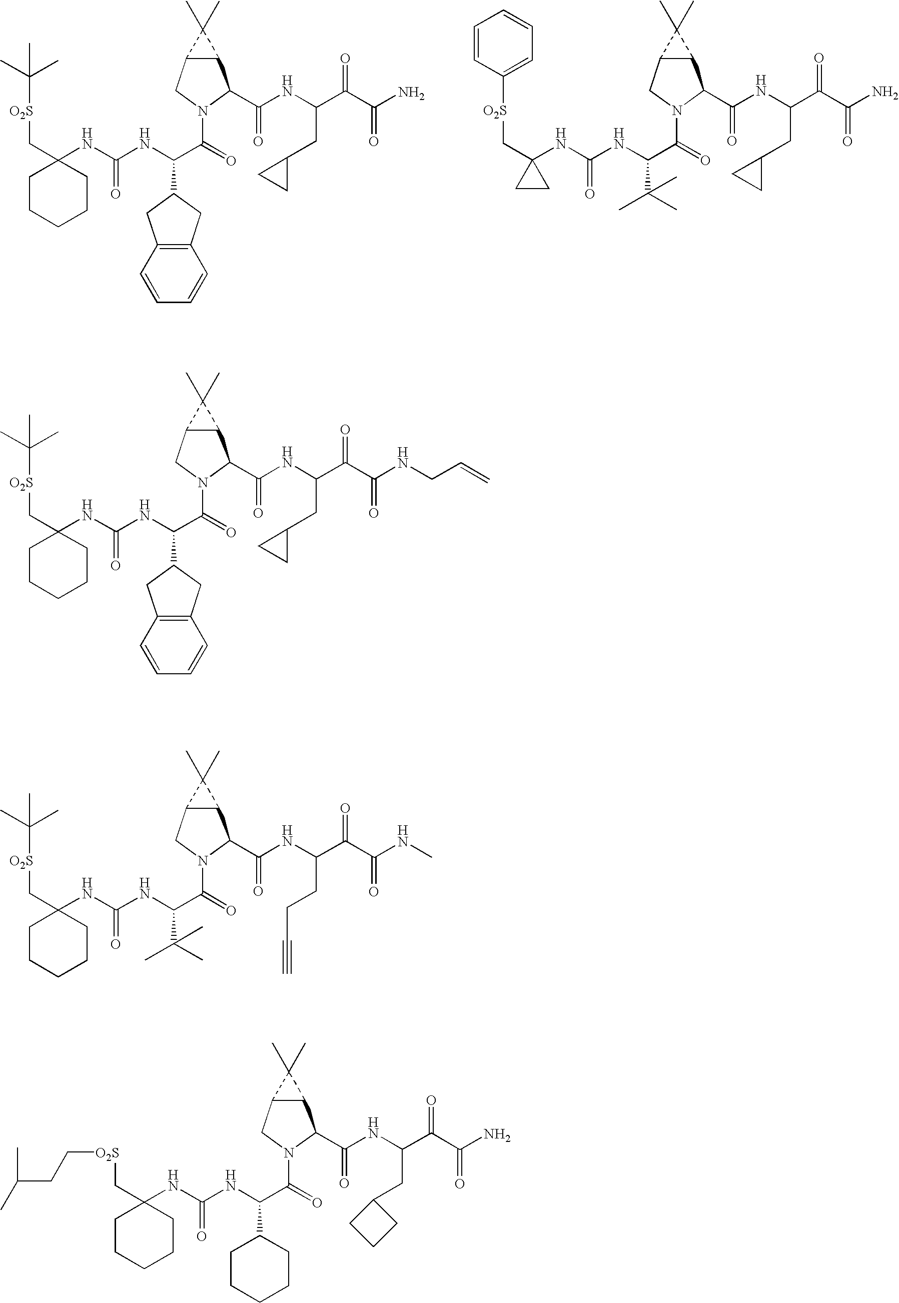 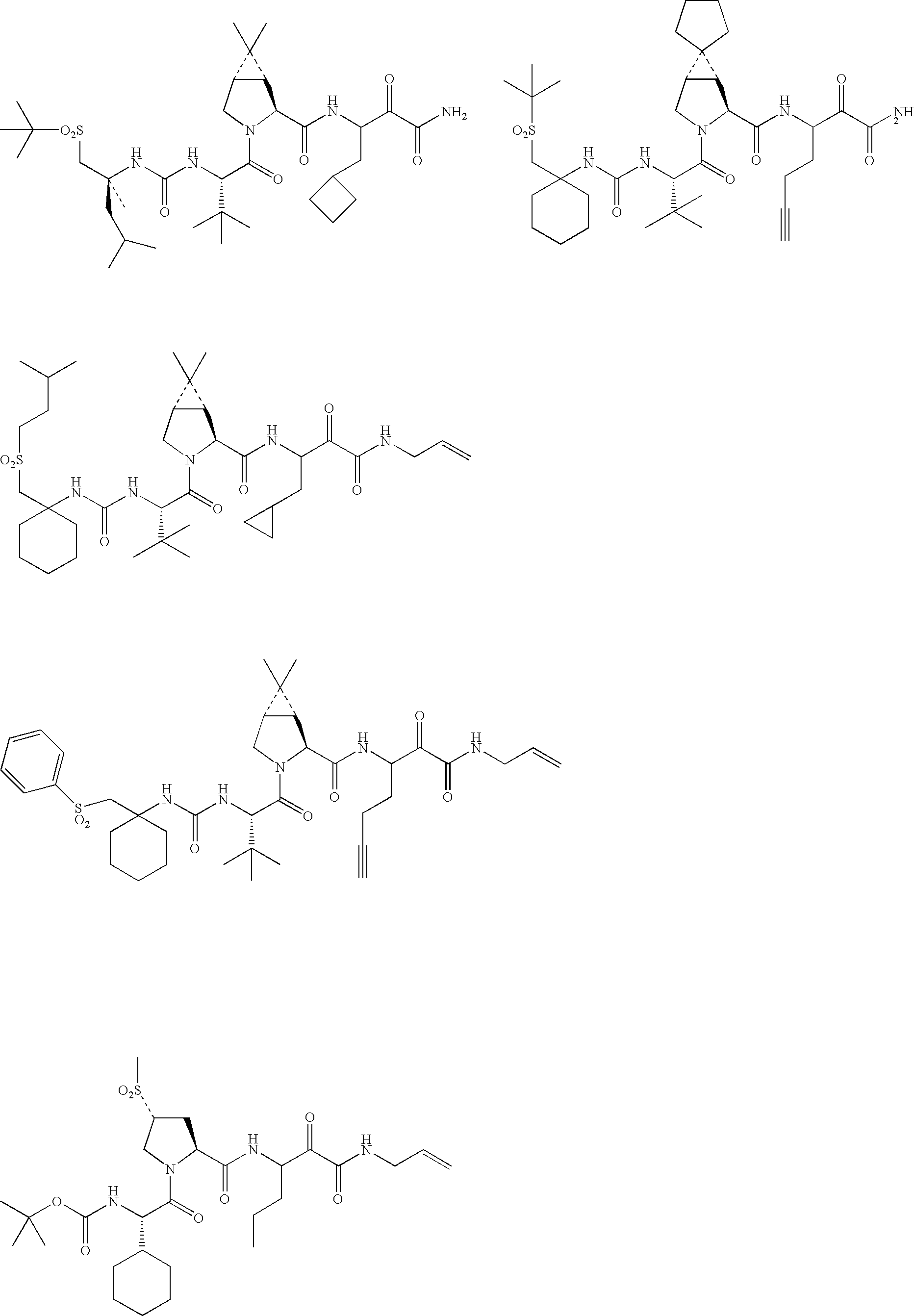 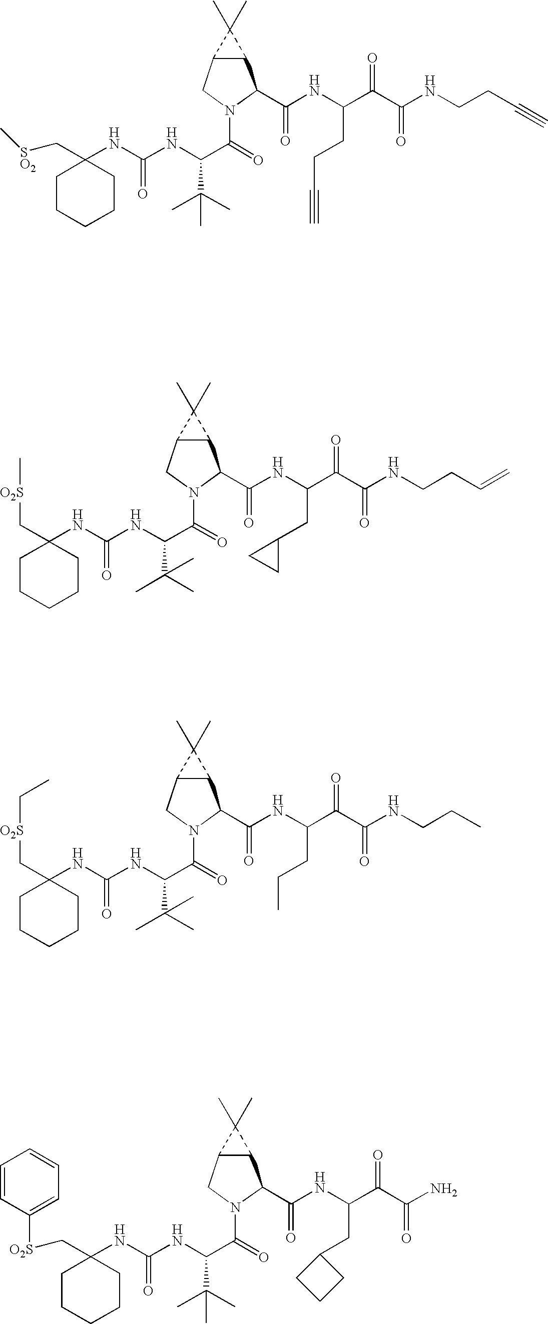 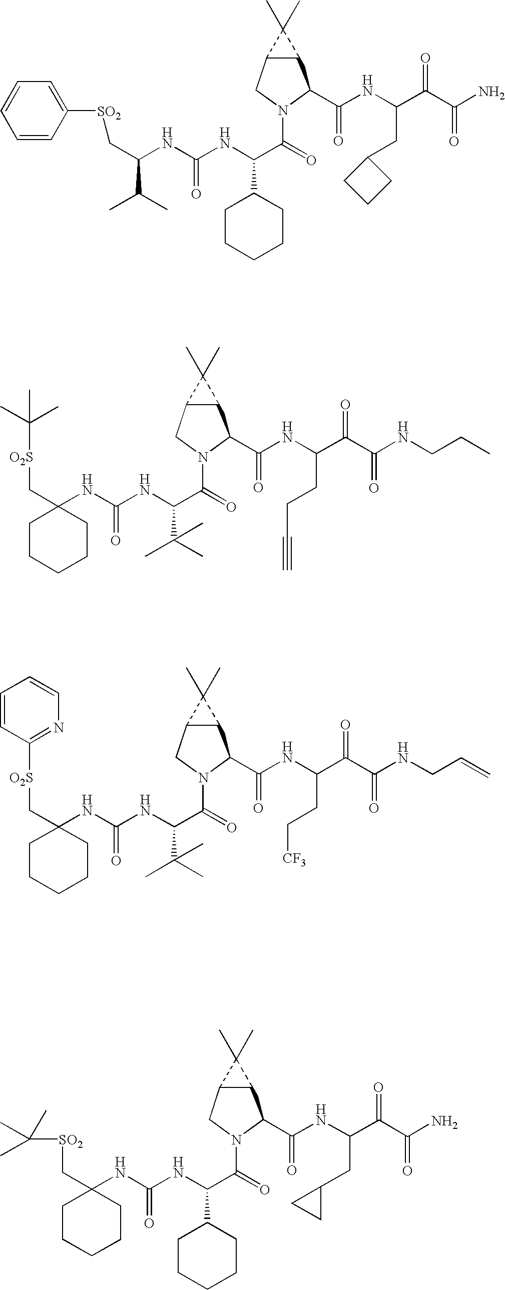 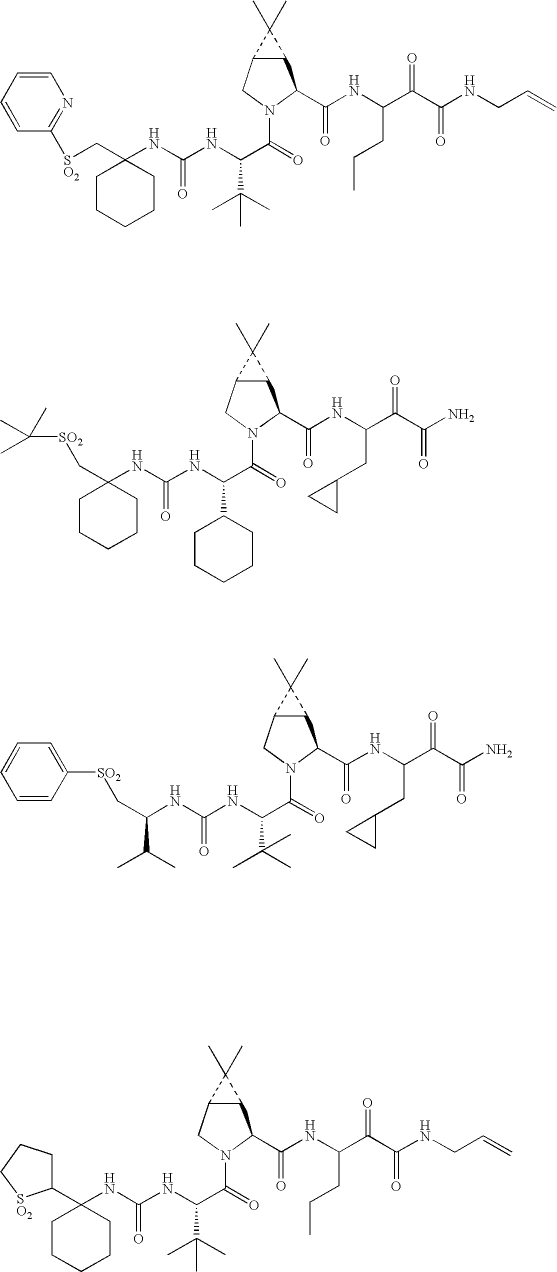 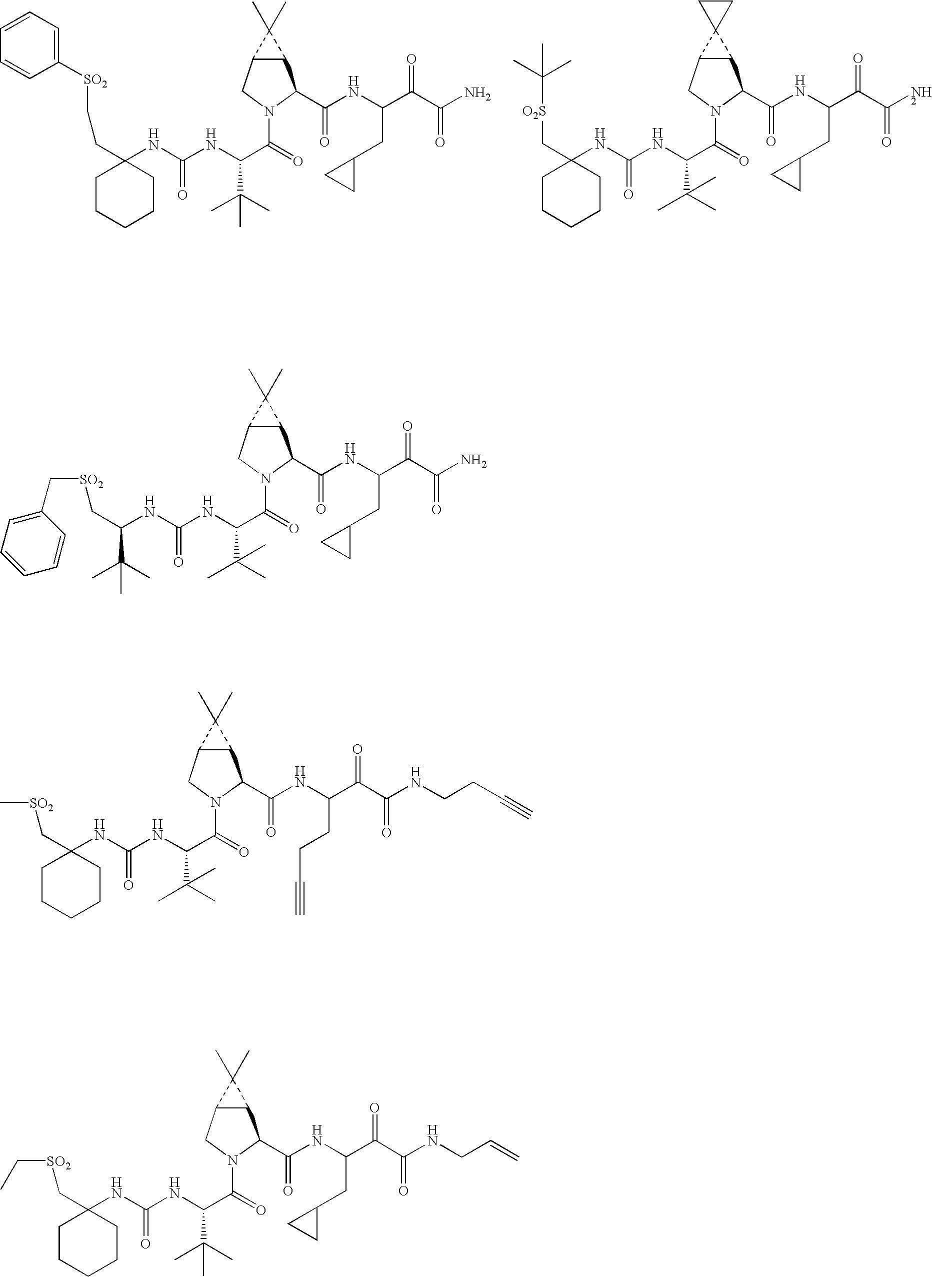 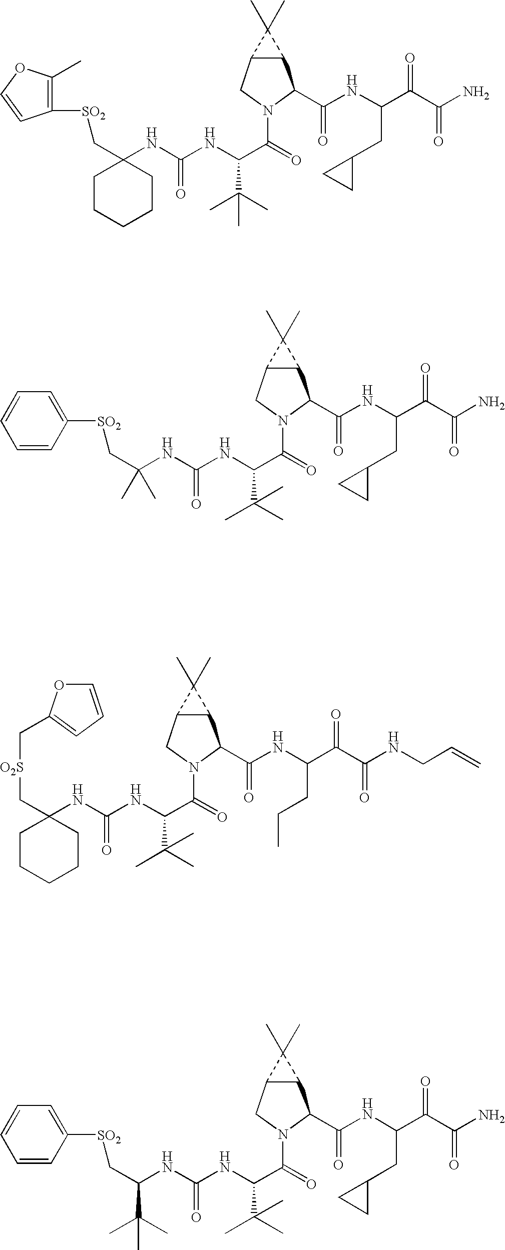 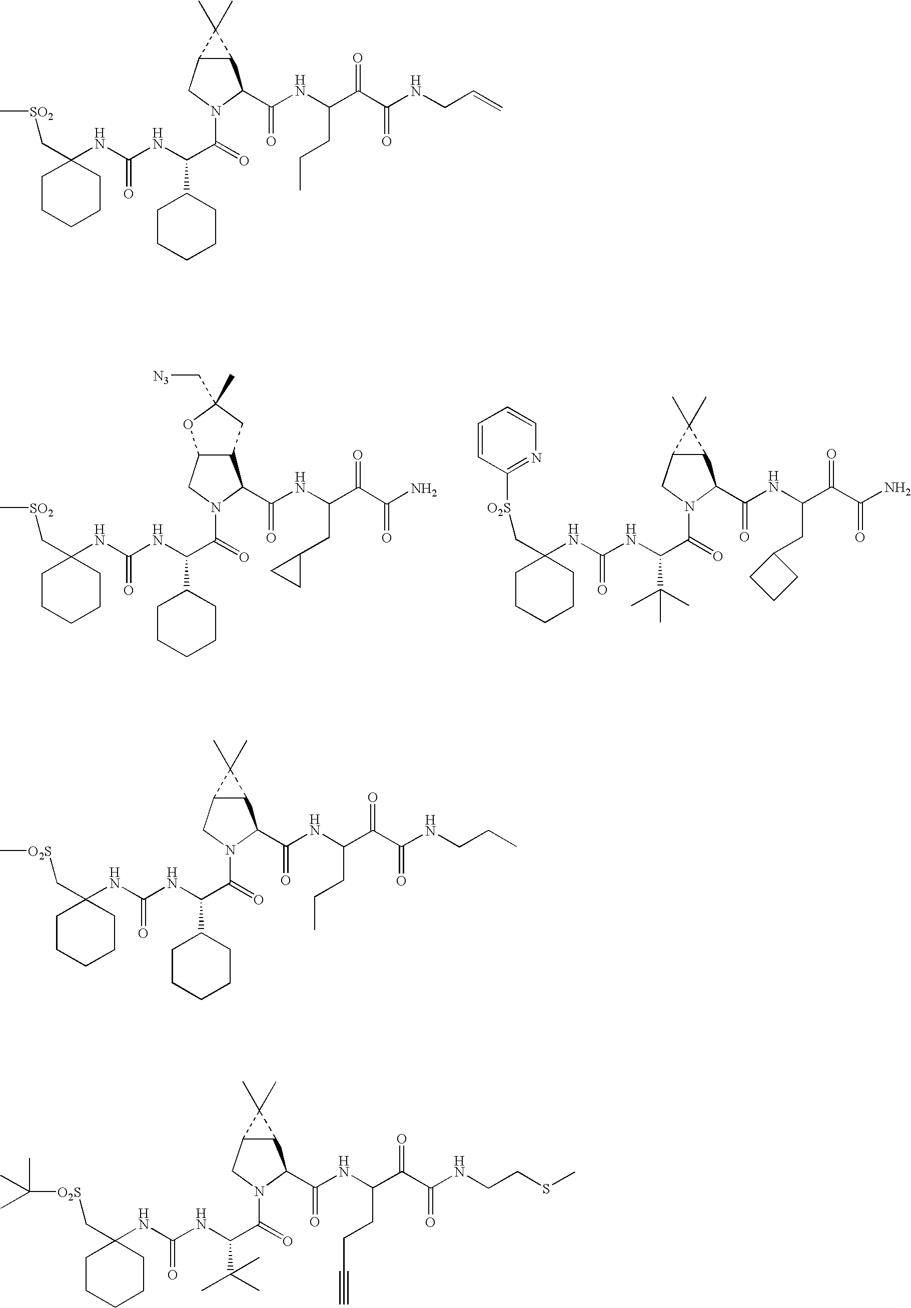 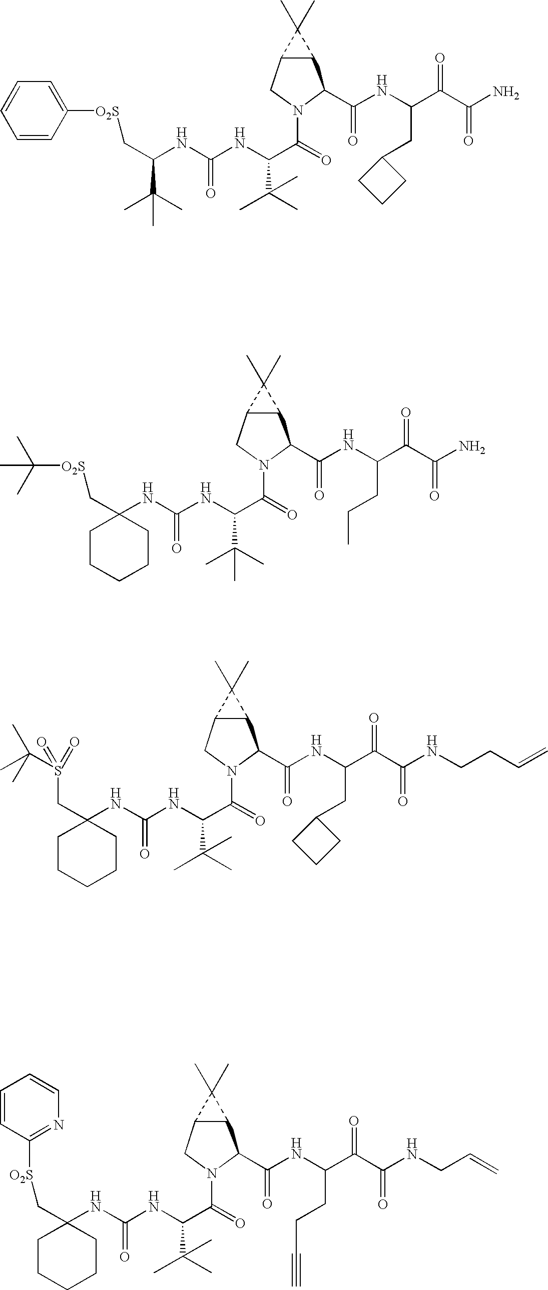 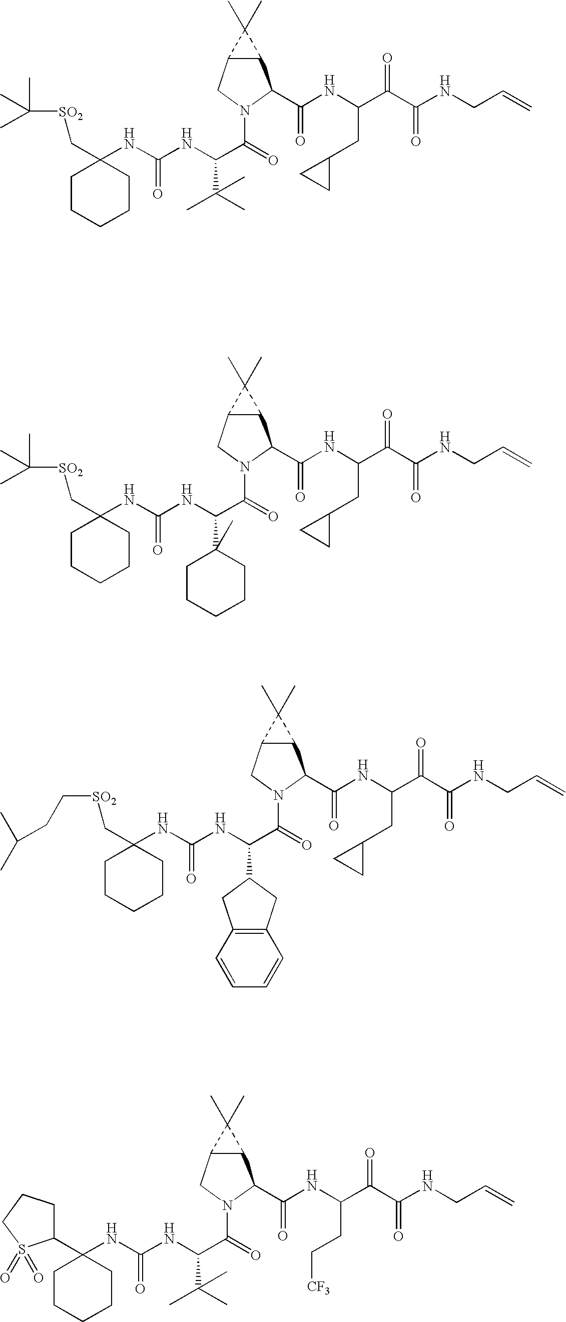 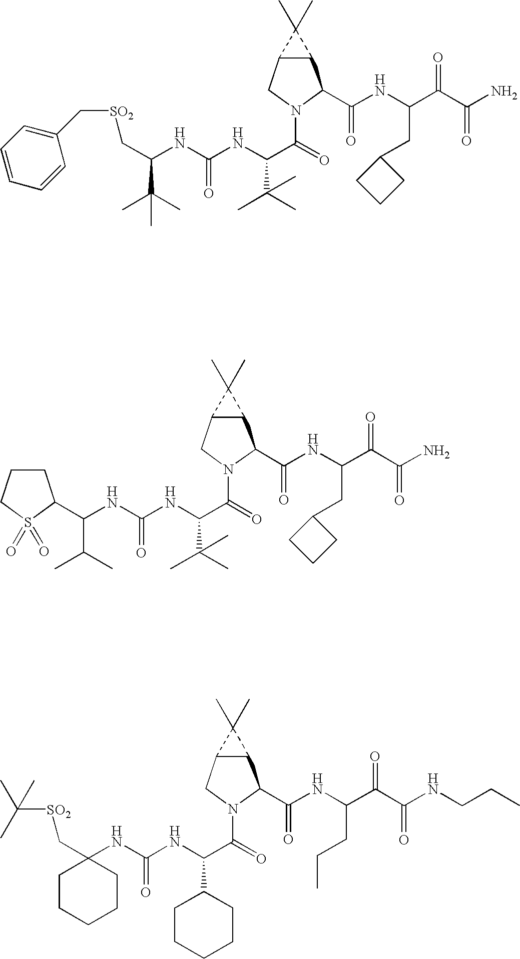 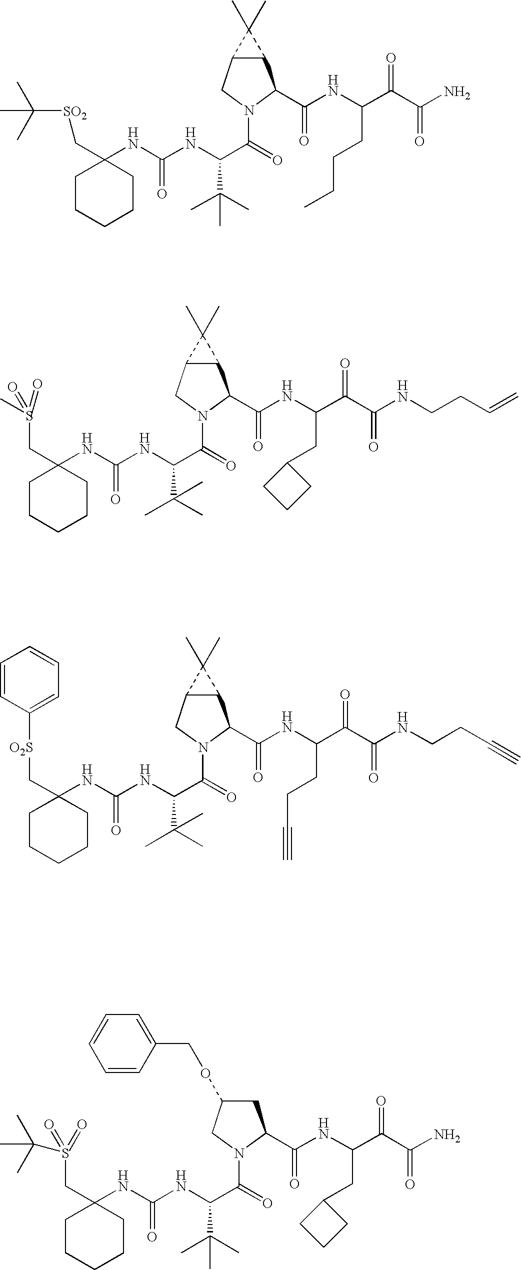 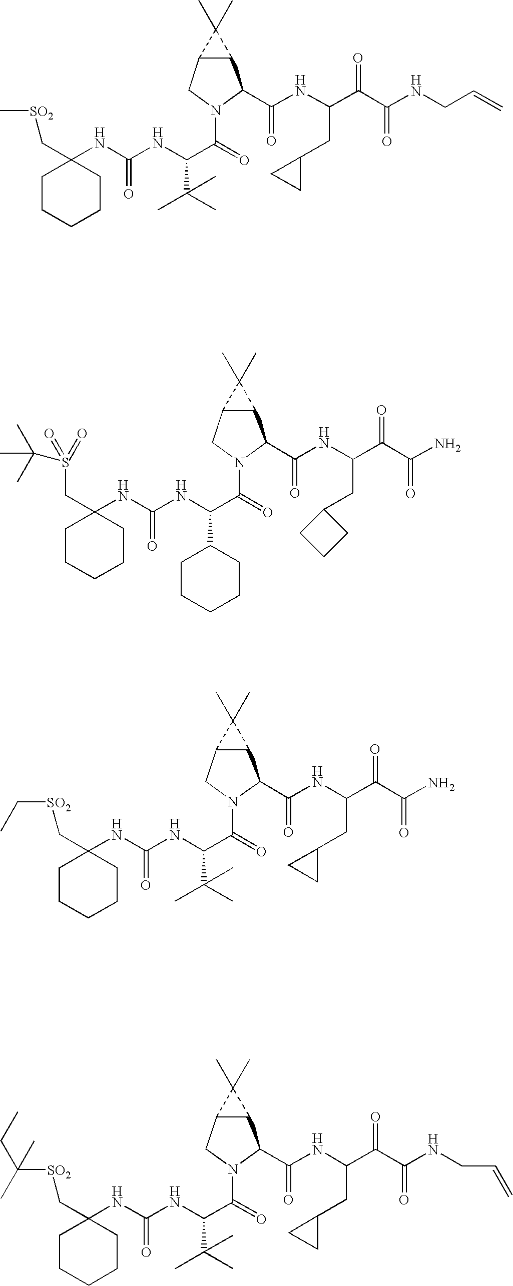 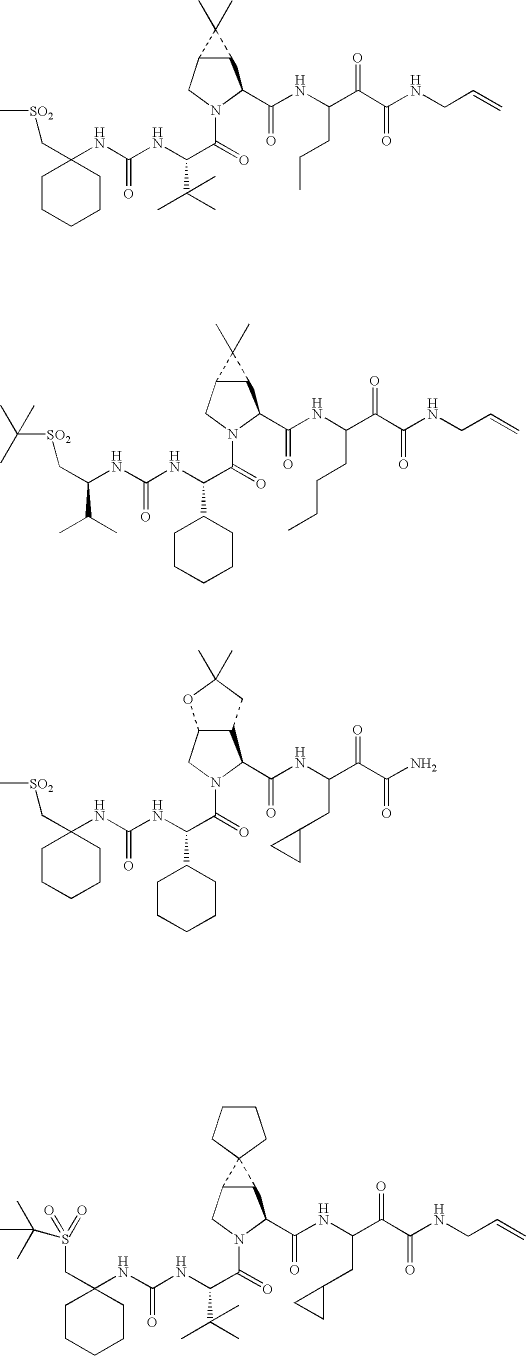 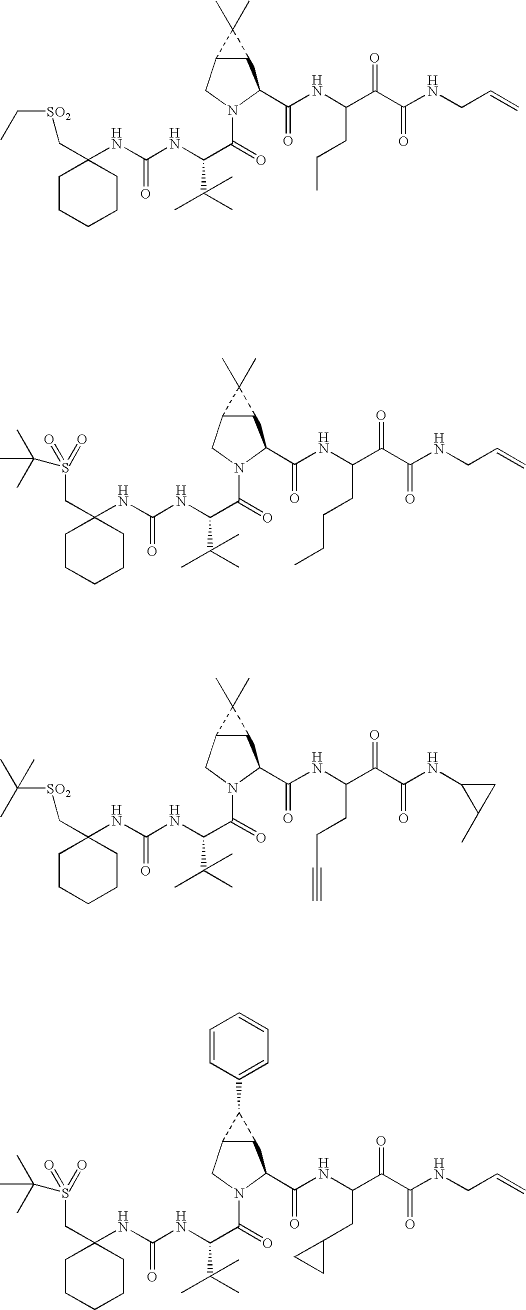 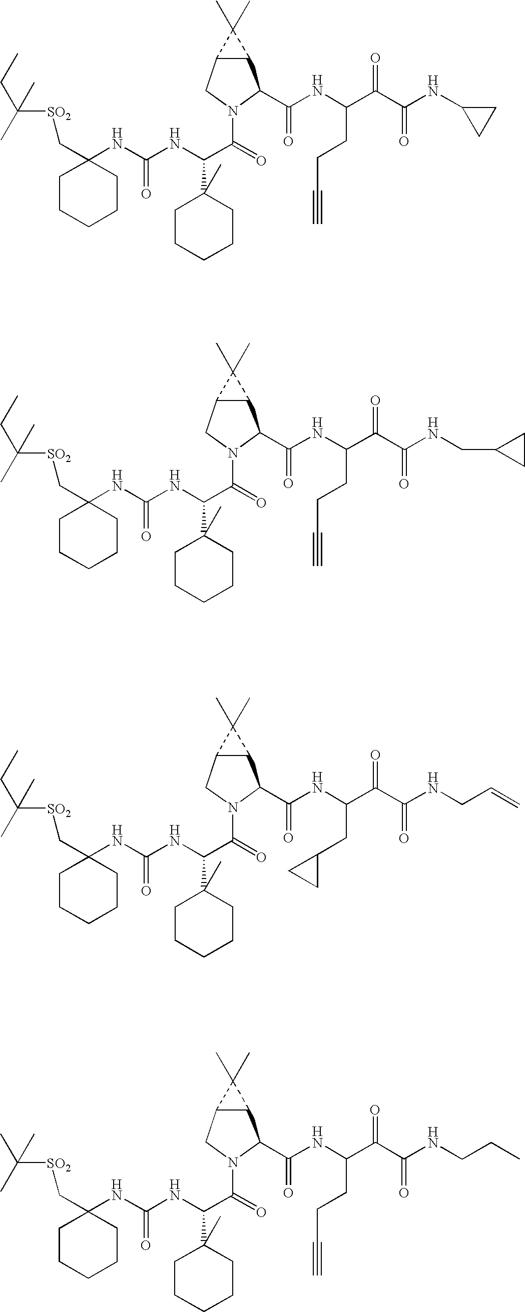 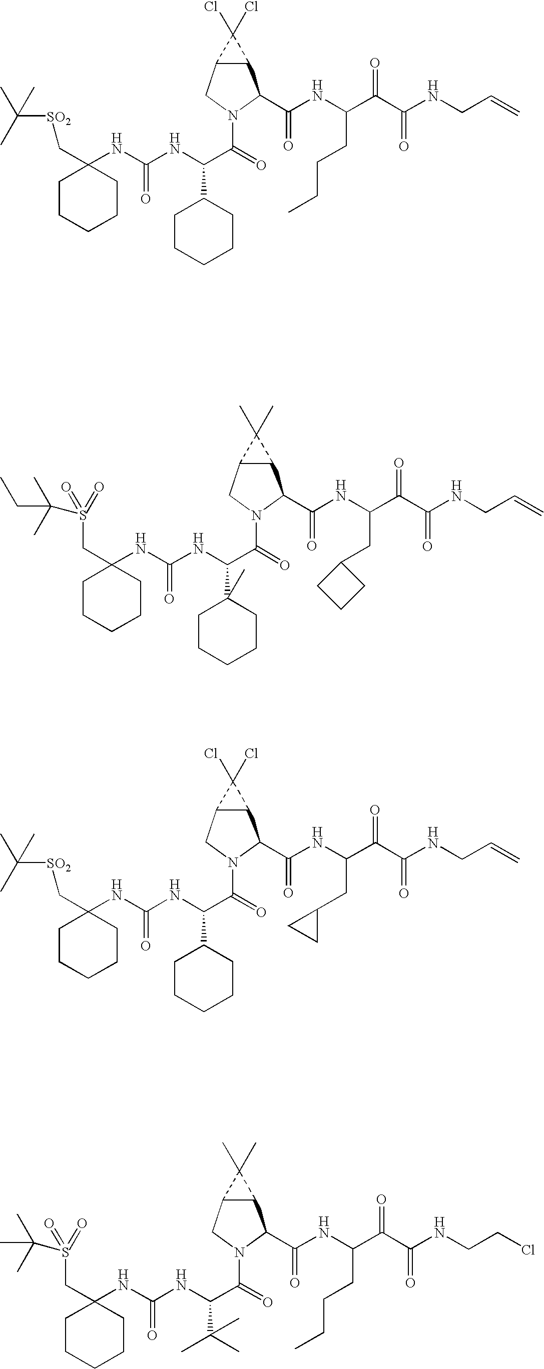 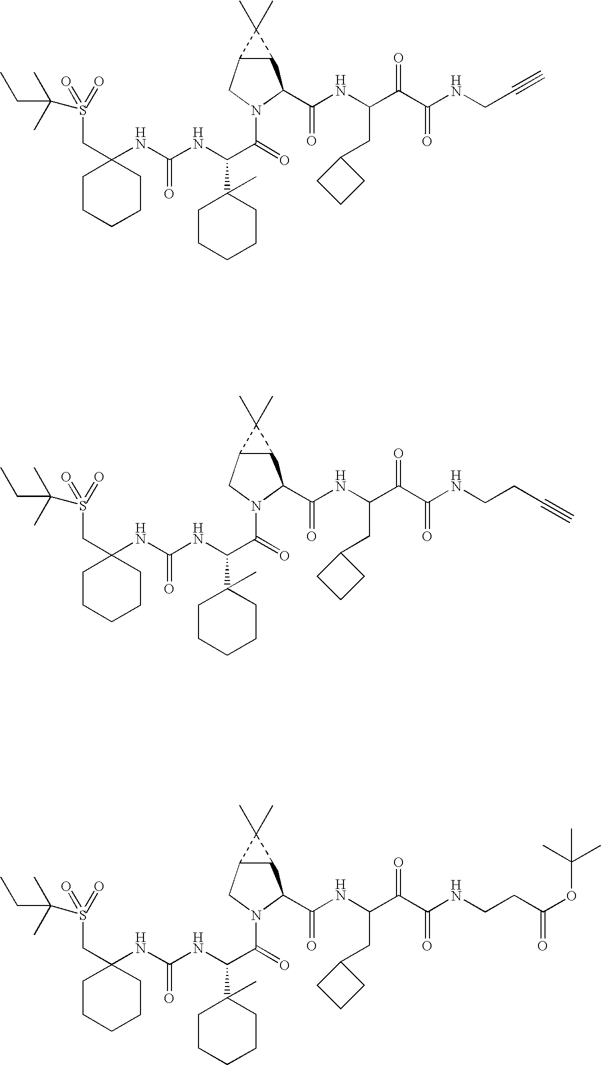 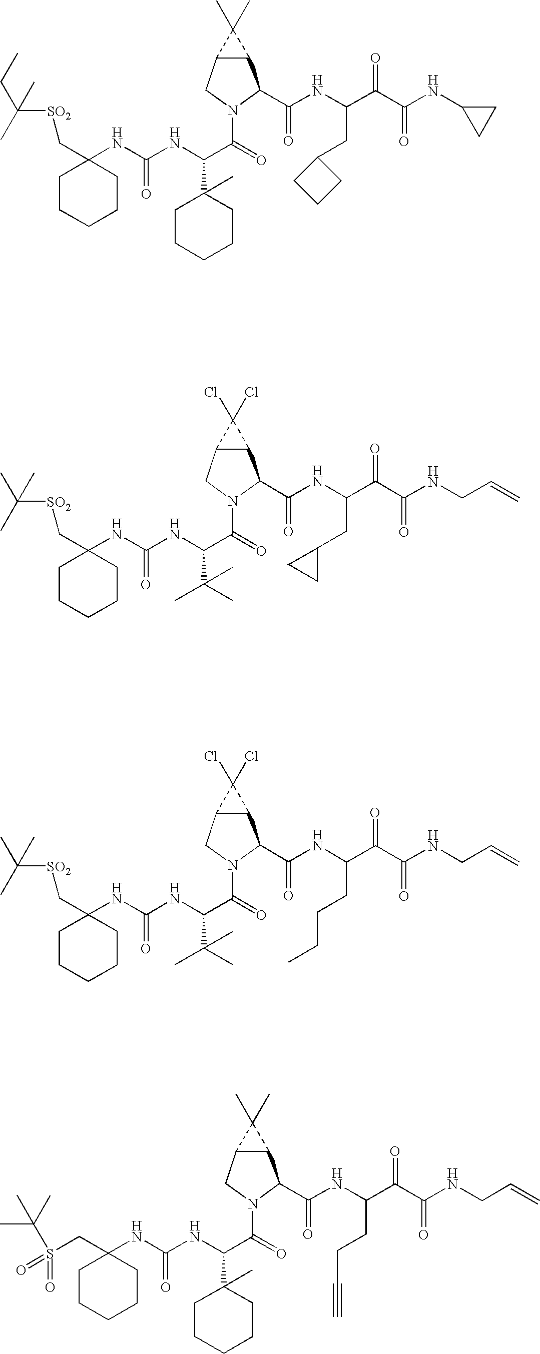 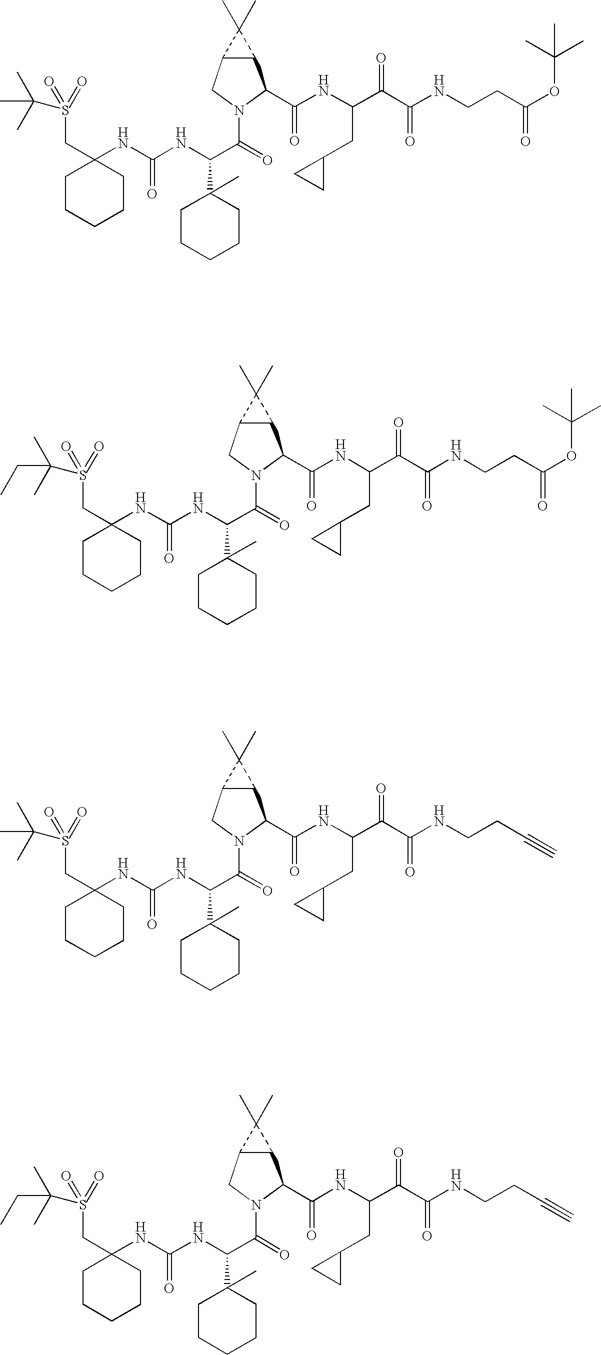 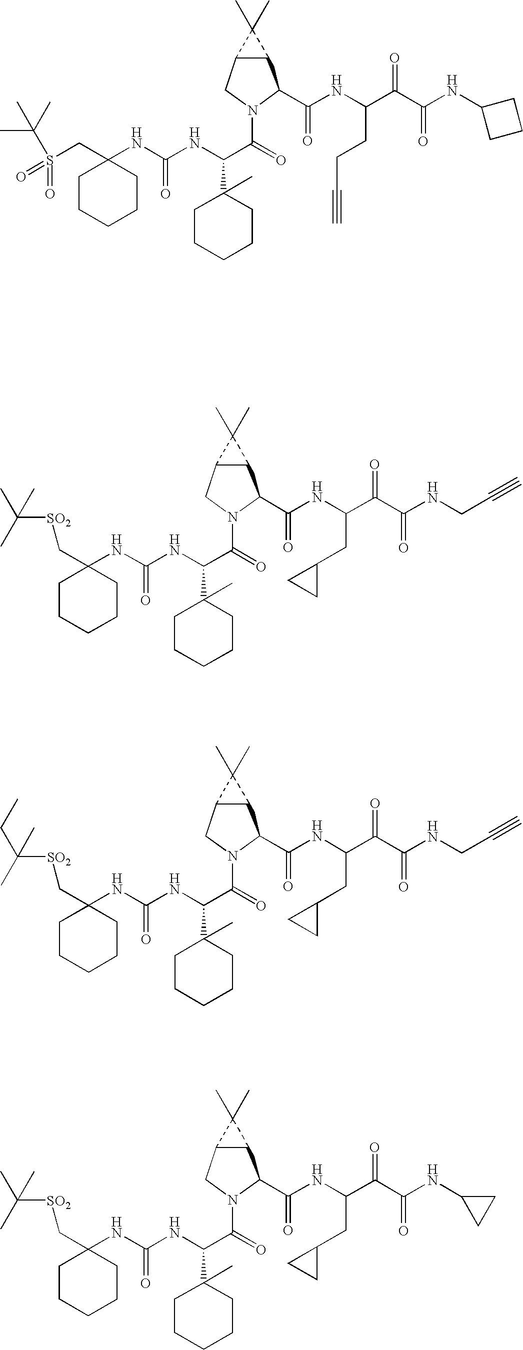 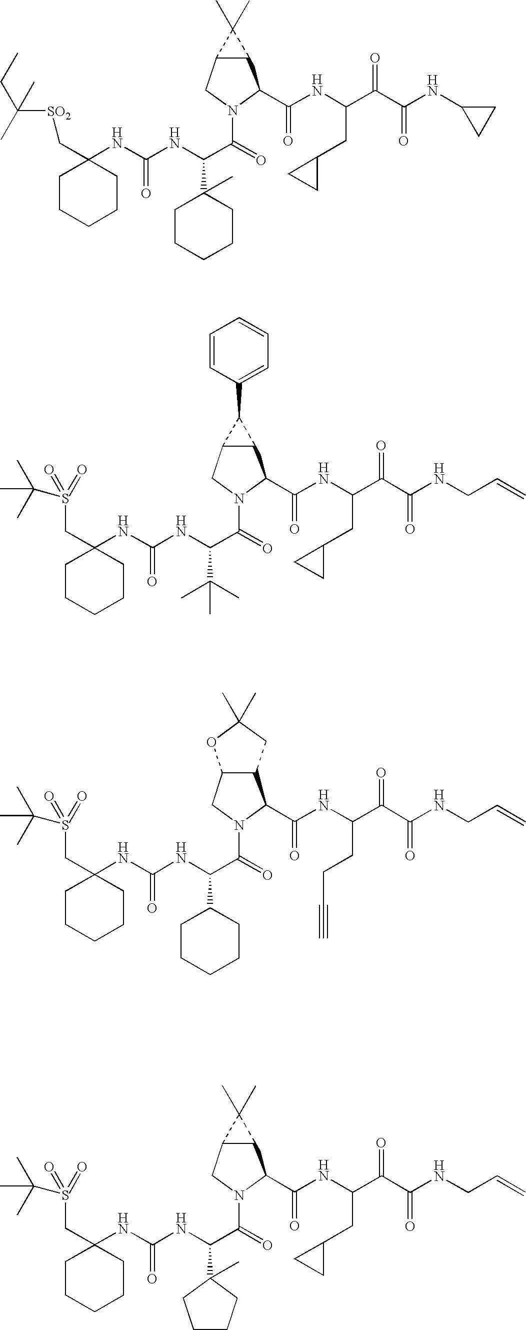 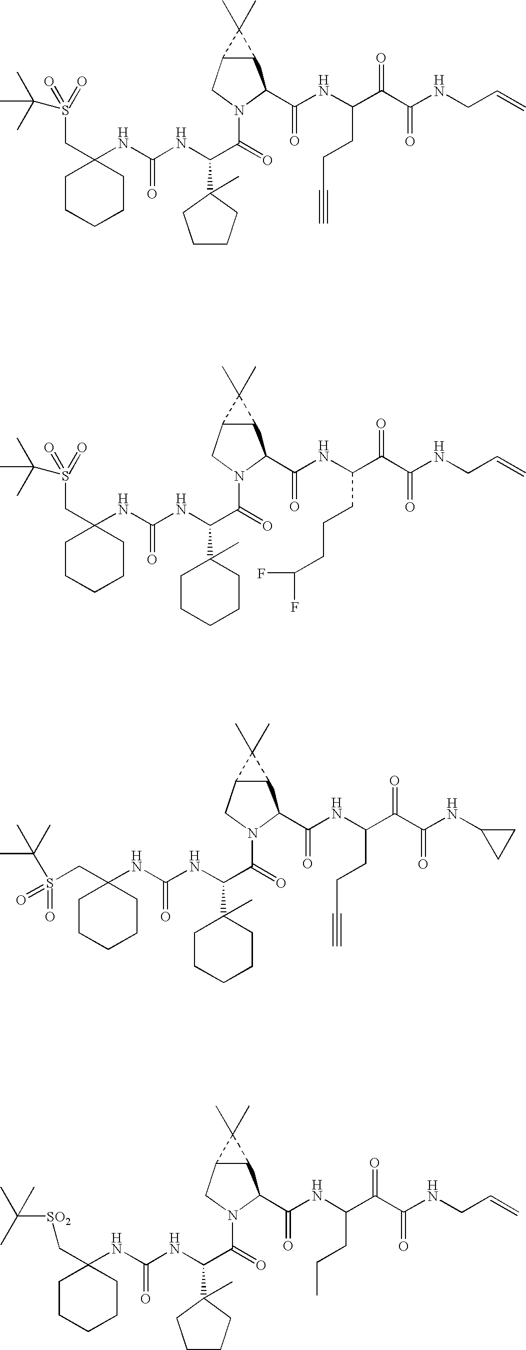 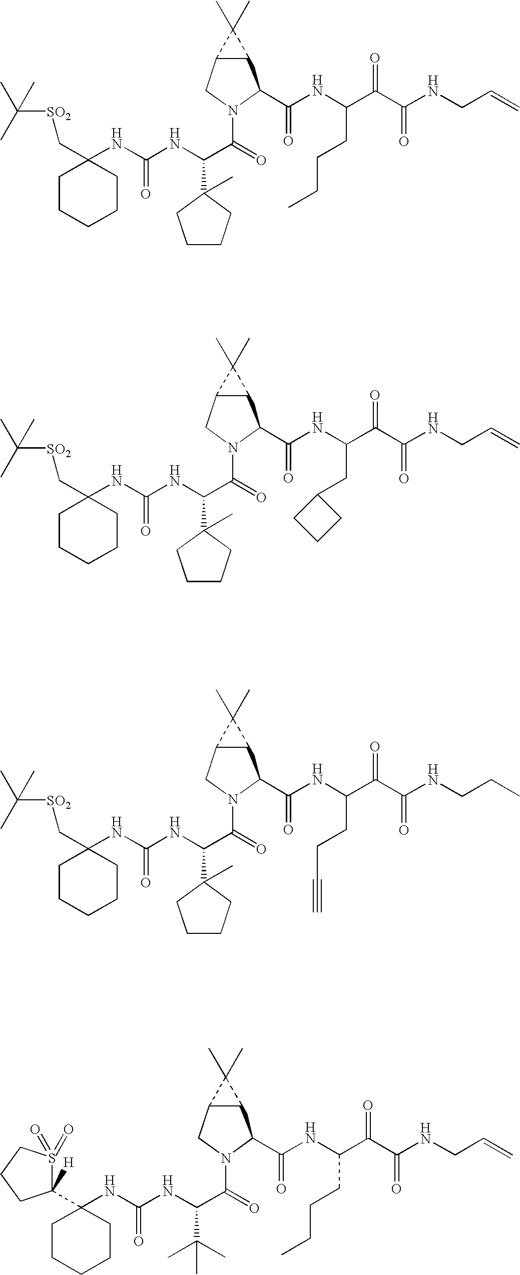 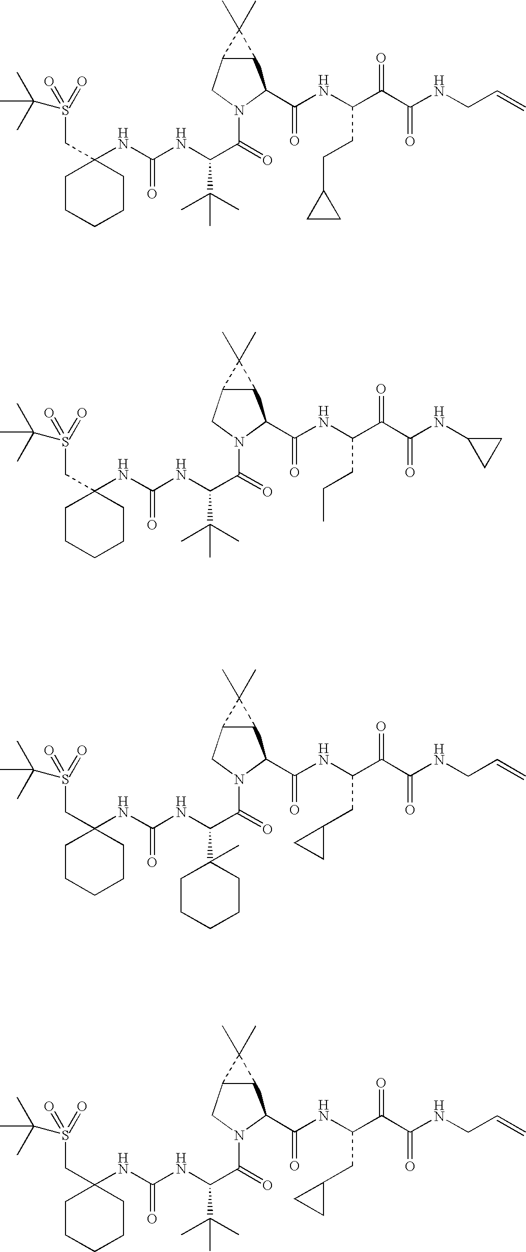 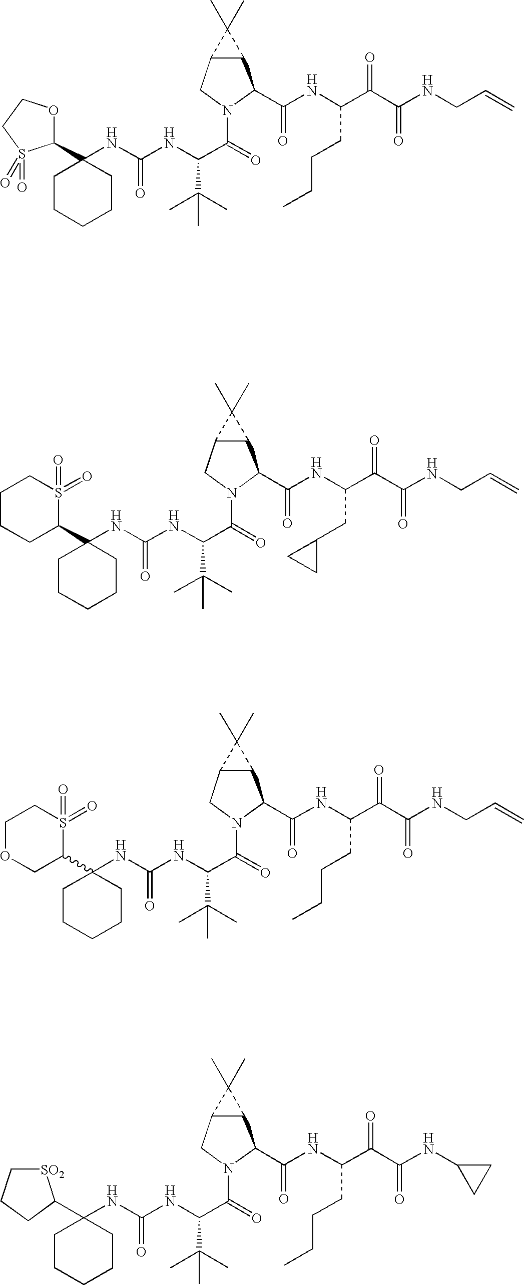 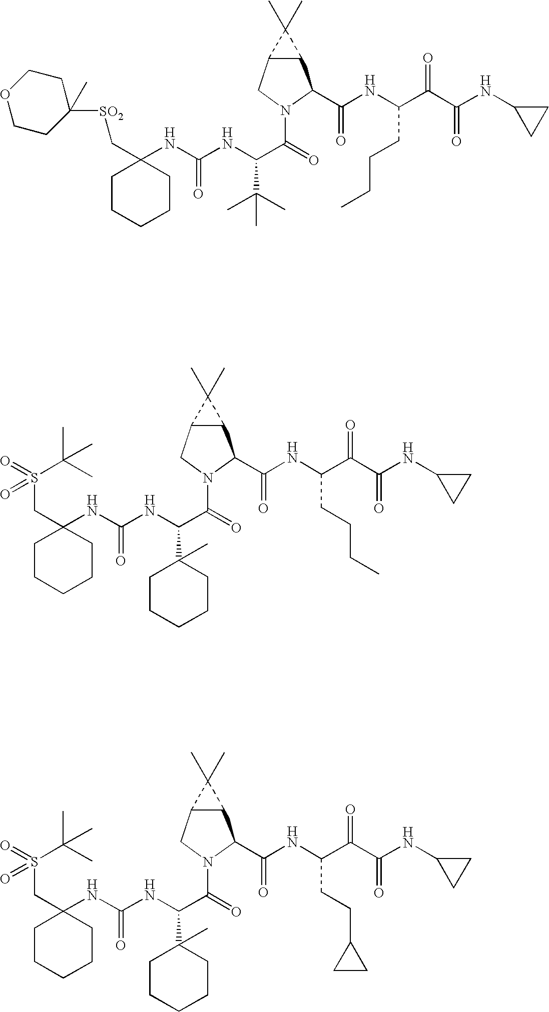 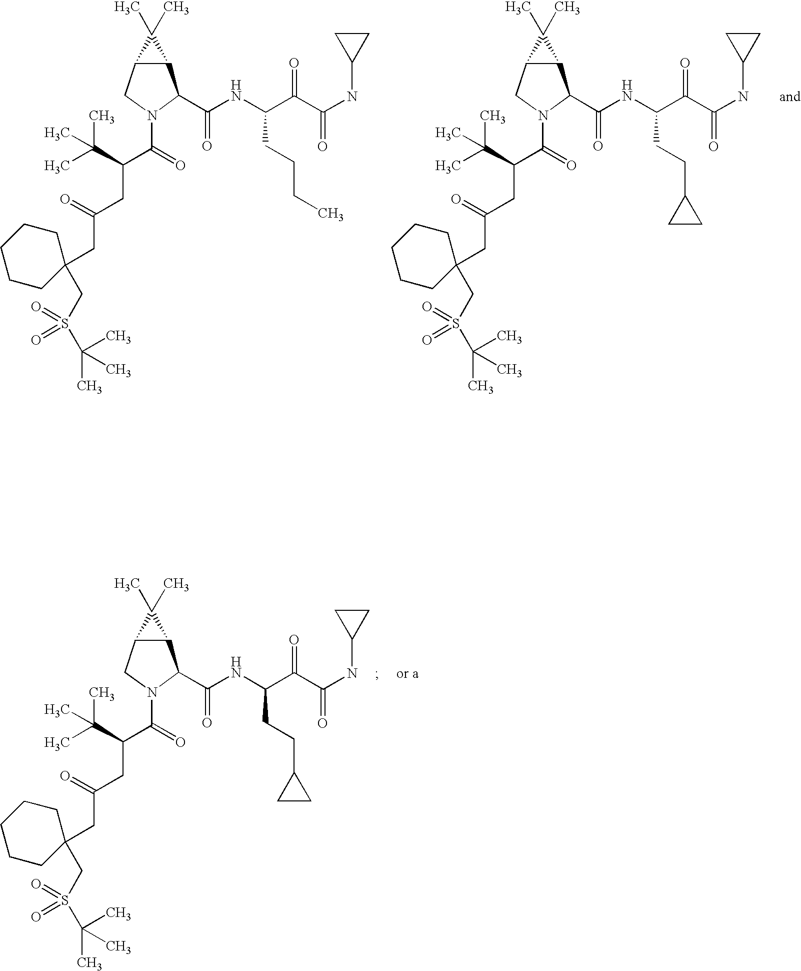
pharmaceutically acceptable salt, solvate or ester thereof.

Compounds of formula XV are disclosed in U.S. patent application Ser. No. 11/007,910 filed Dec. 9, 2004. The preparation of these compounds is disclosed in the experimental section of this application set forth hereinbelow.

Non-limiting examples of certain compounds disclosed in U.S. patent application Ser. No. 11/007,910 are: 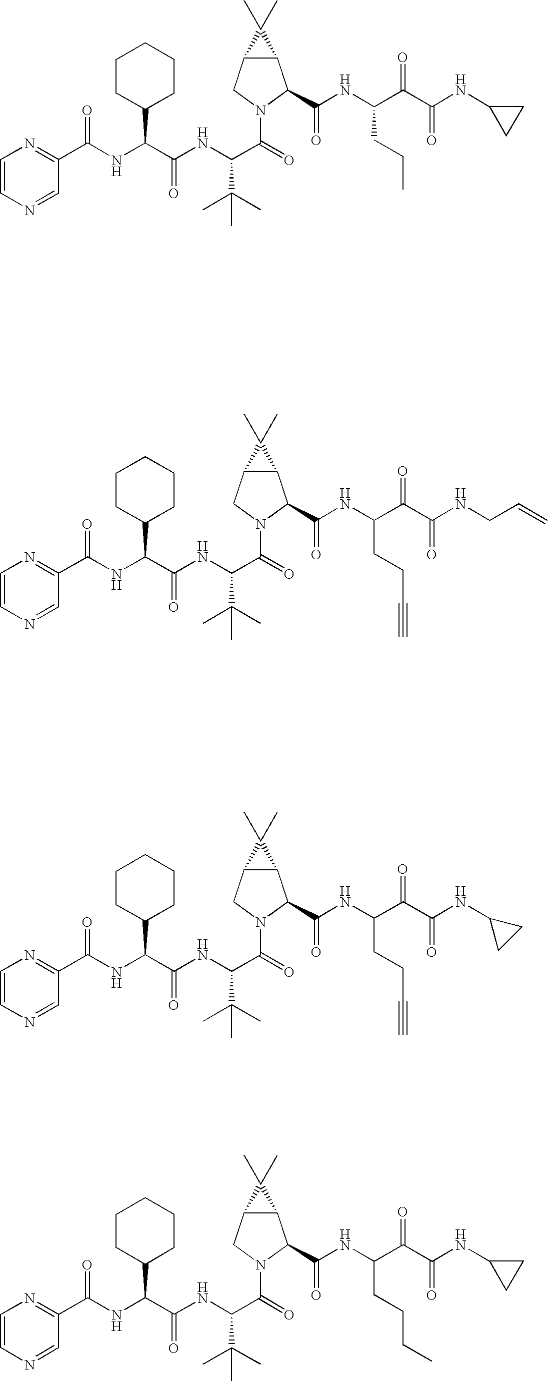 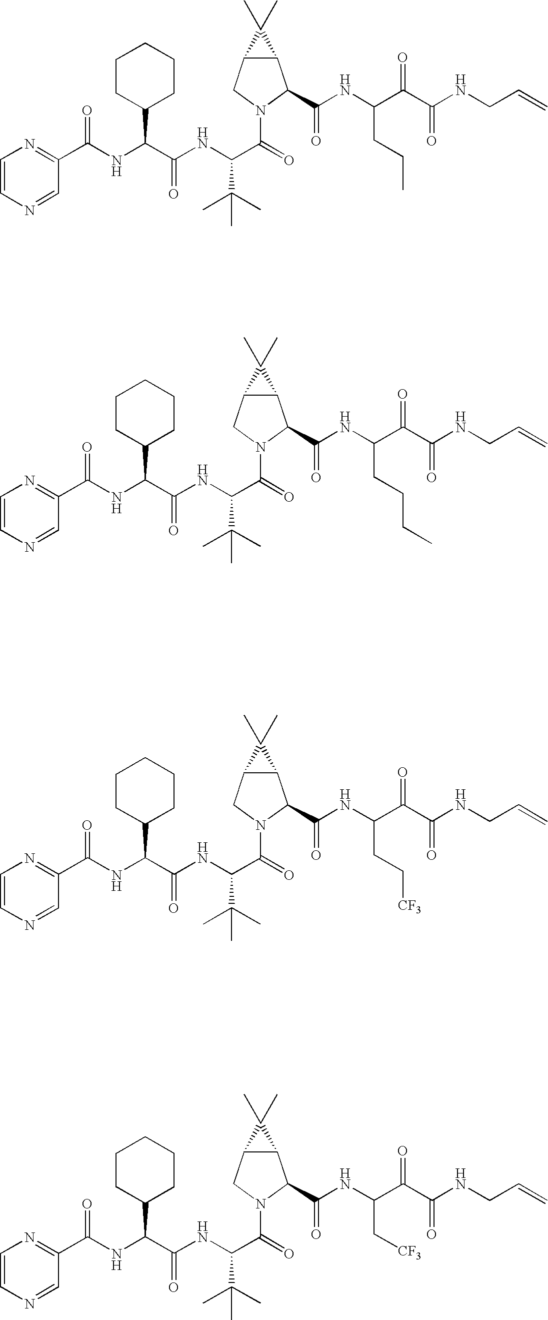 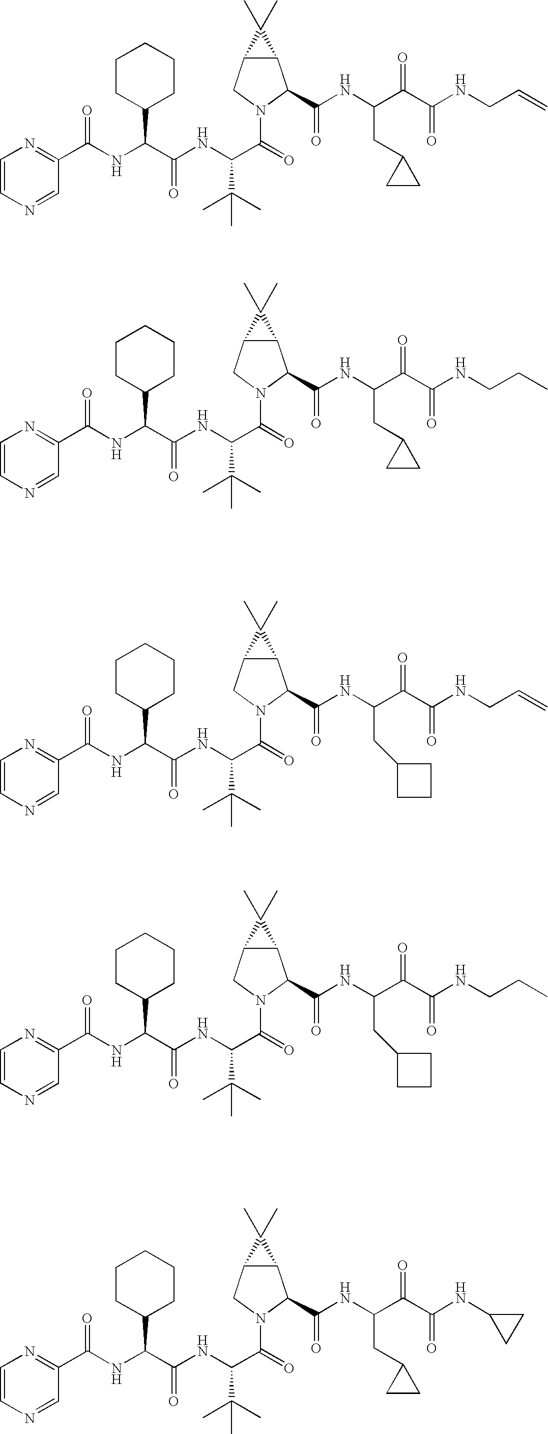 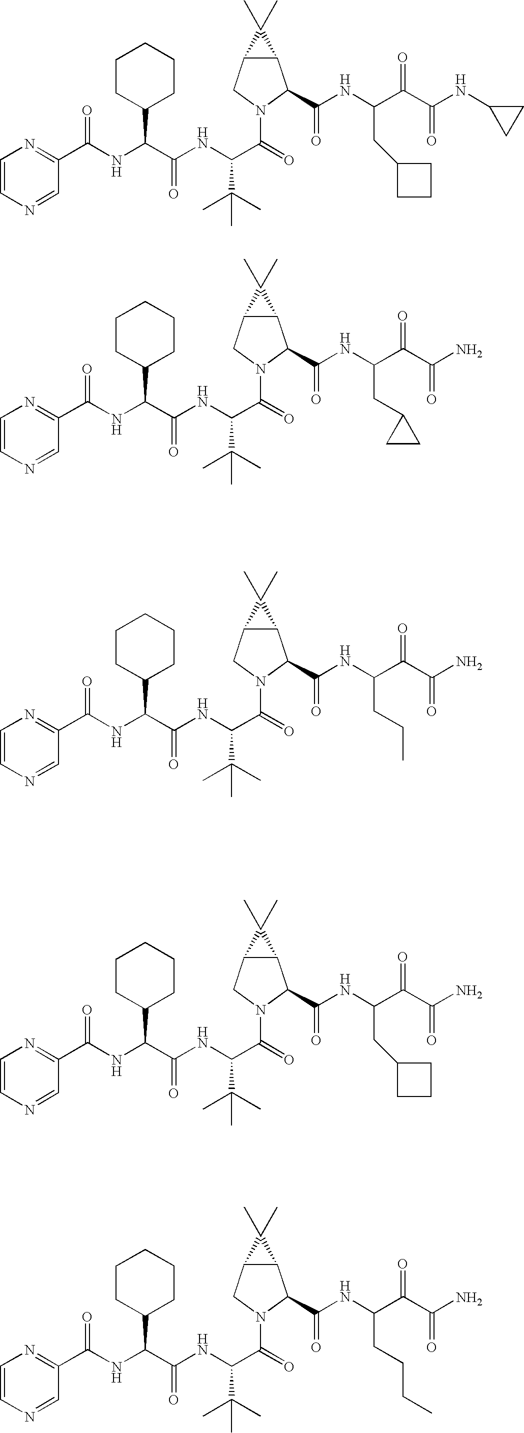 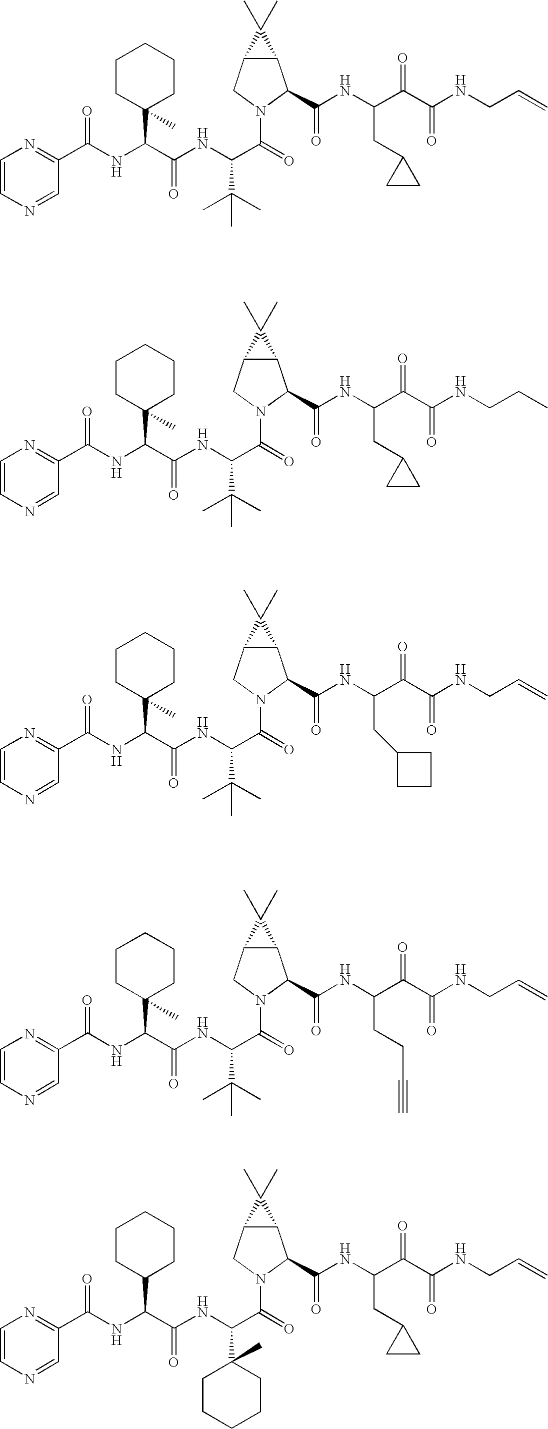 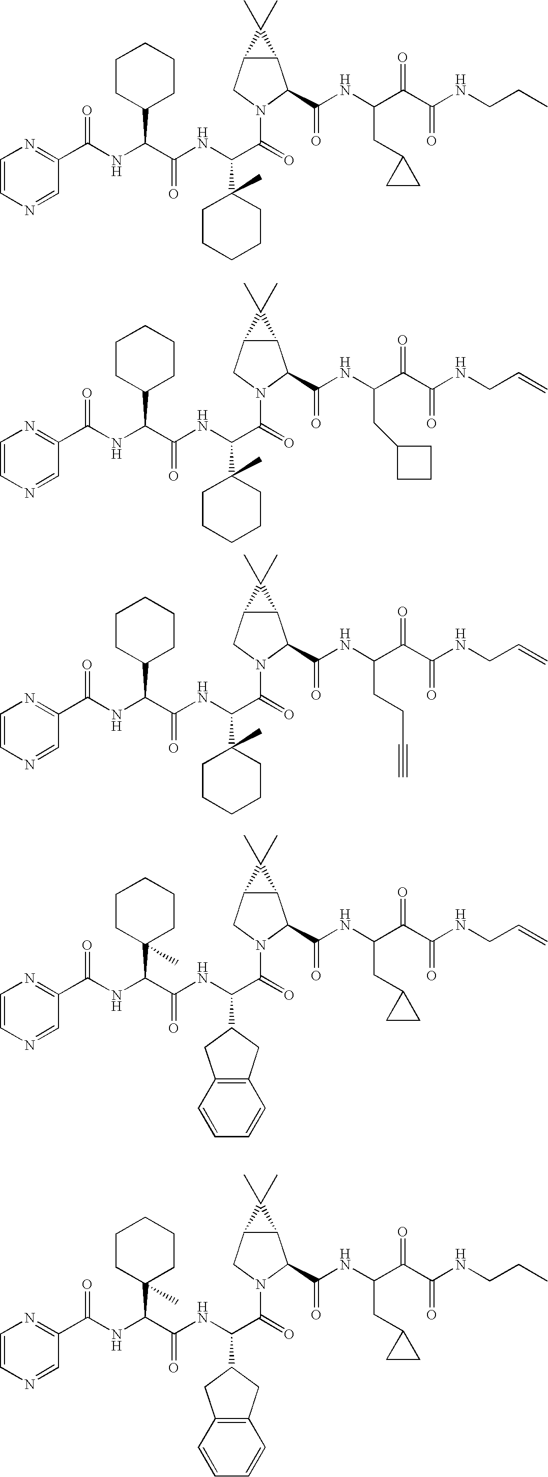 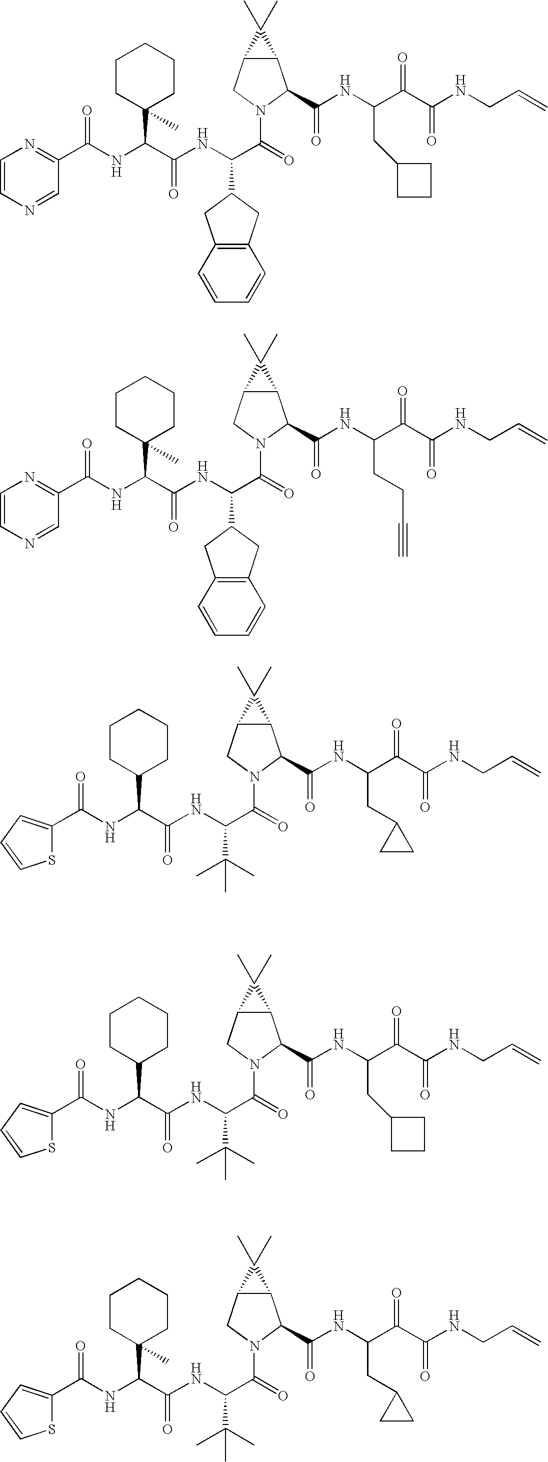 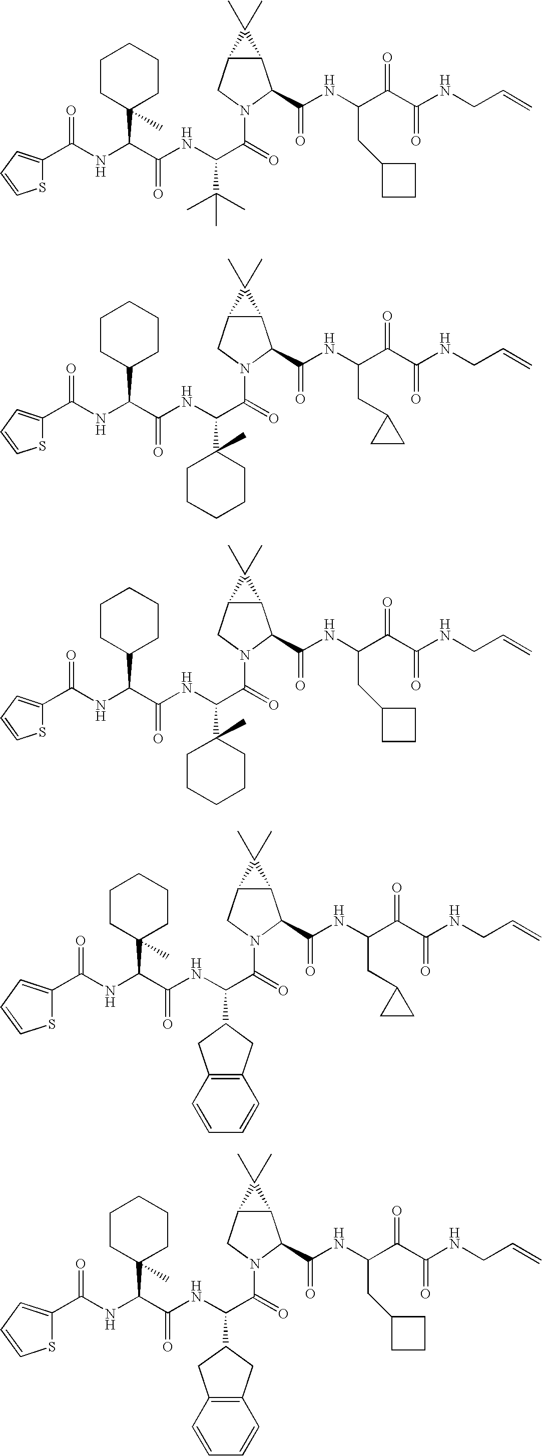 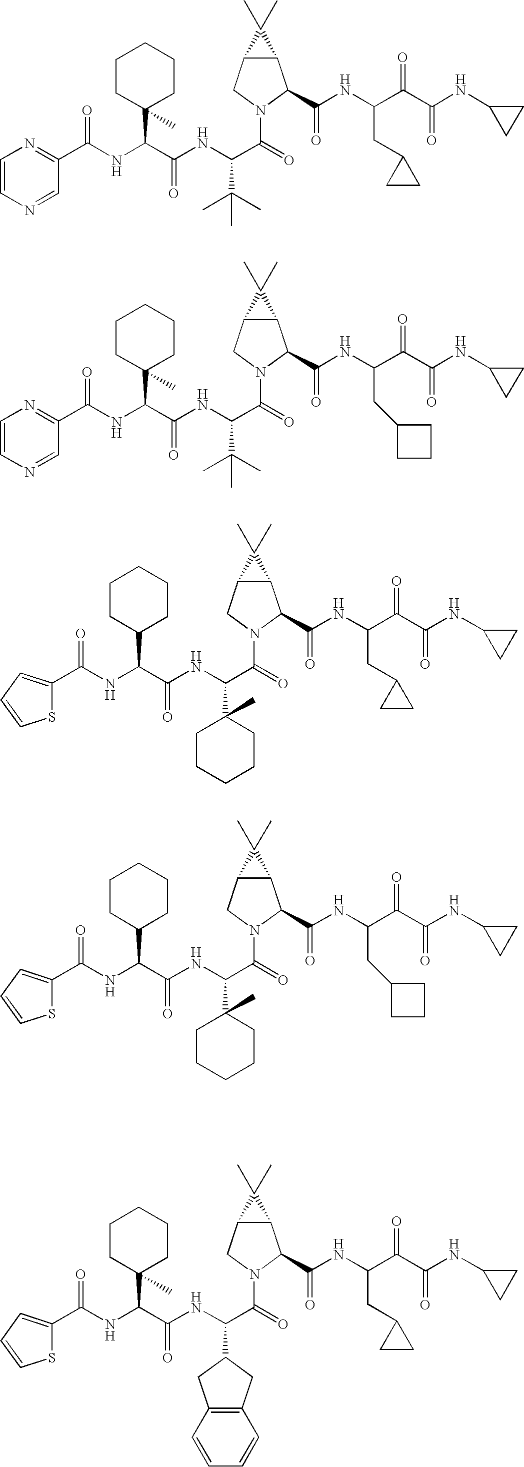 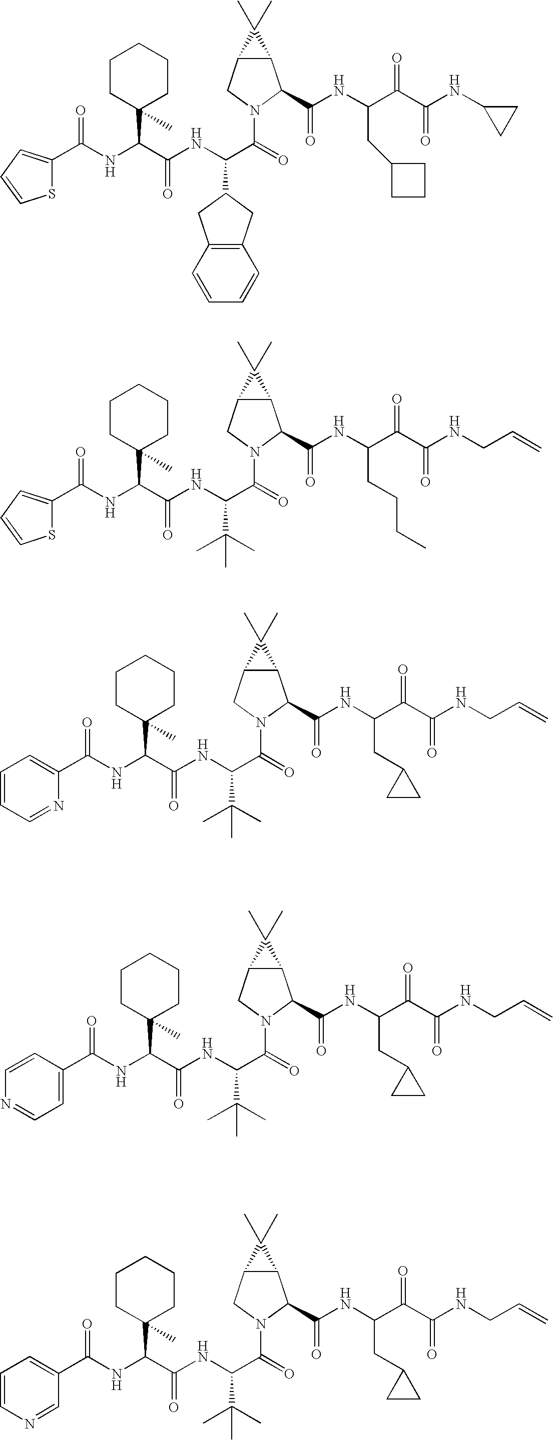 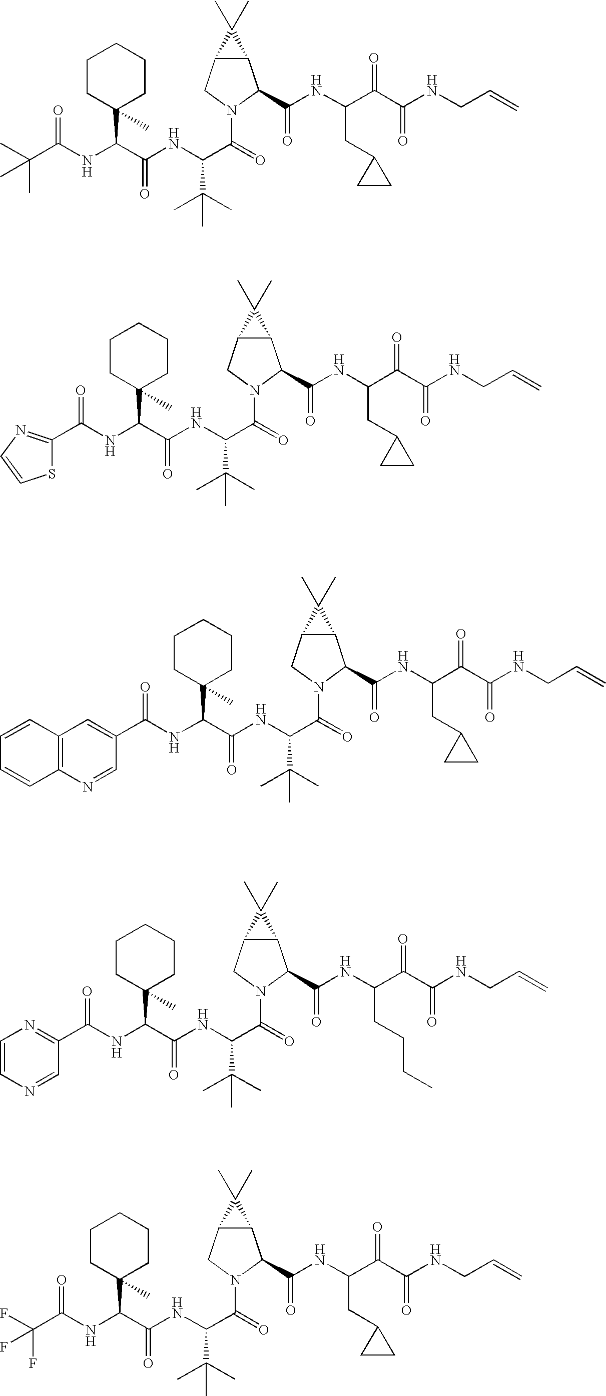 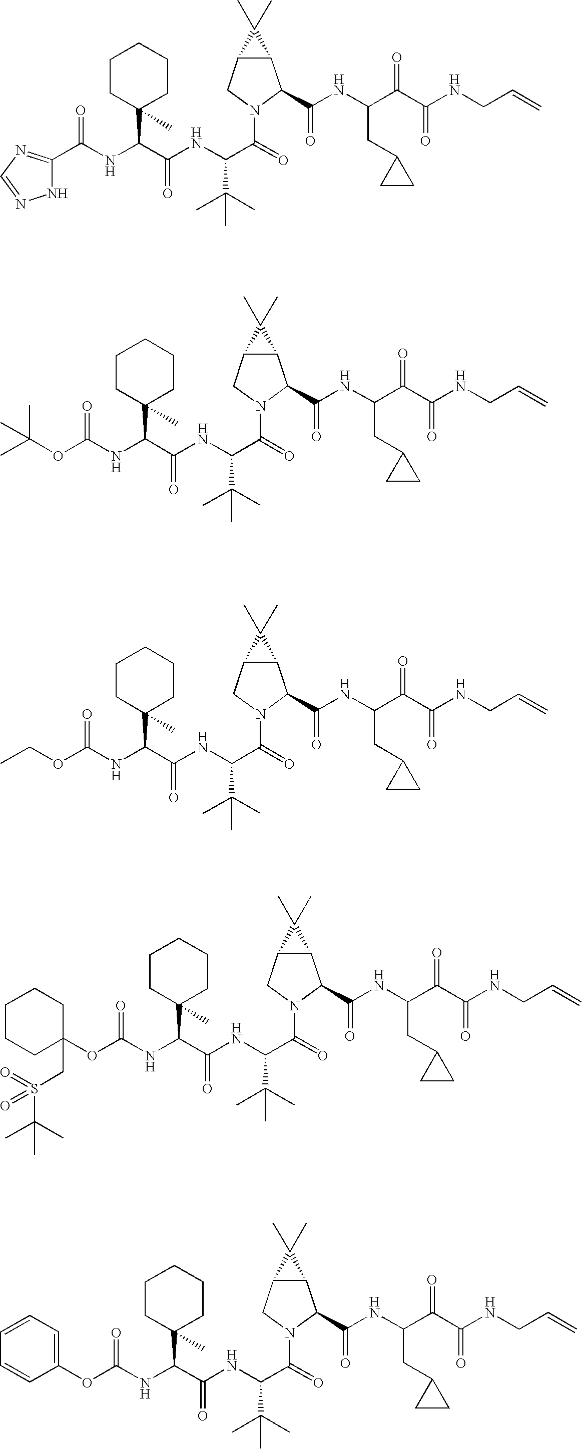 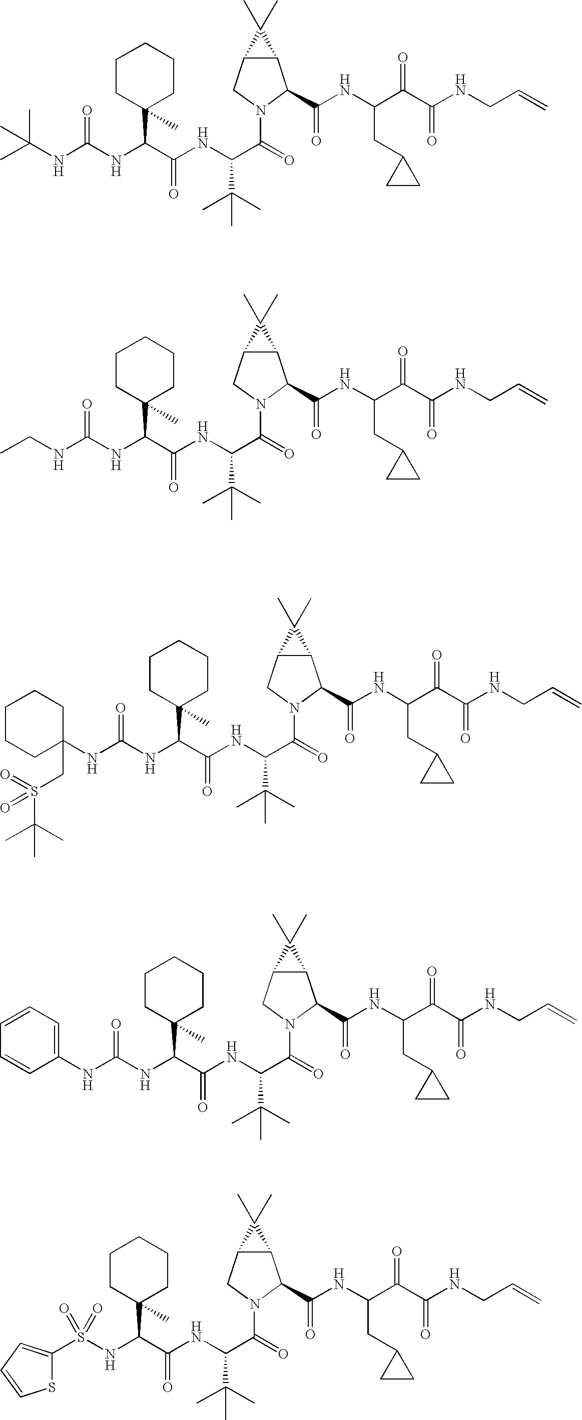 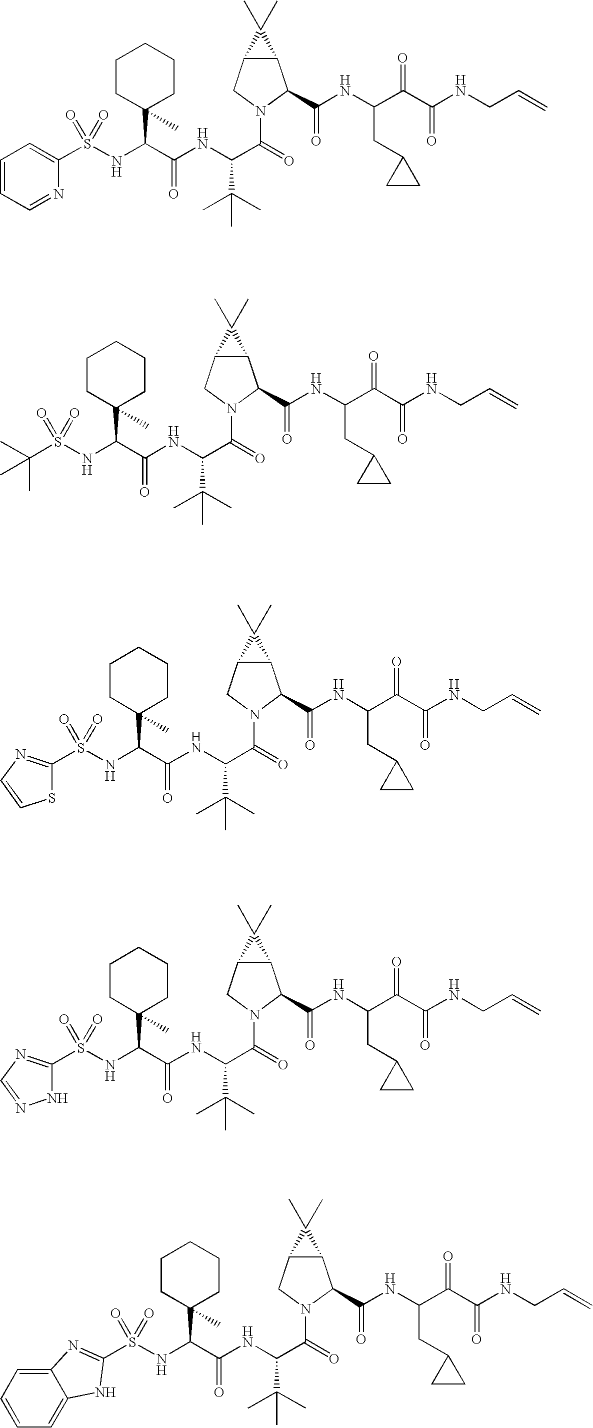 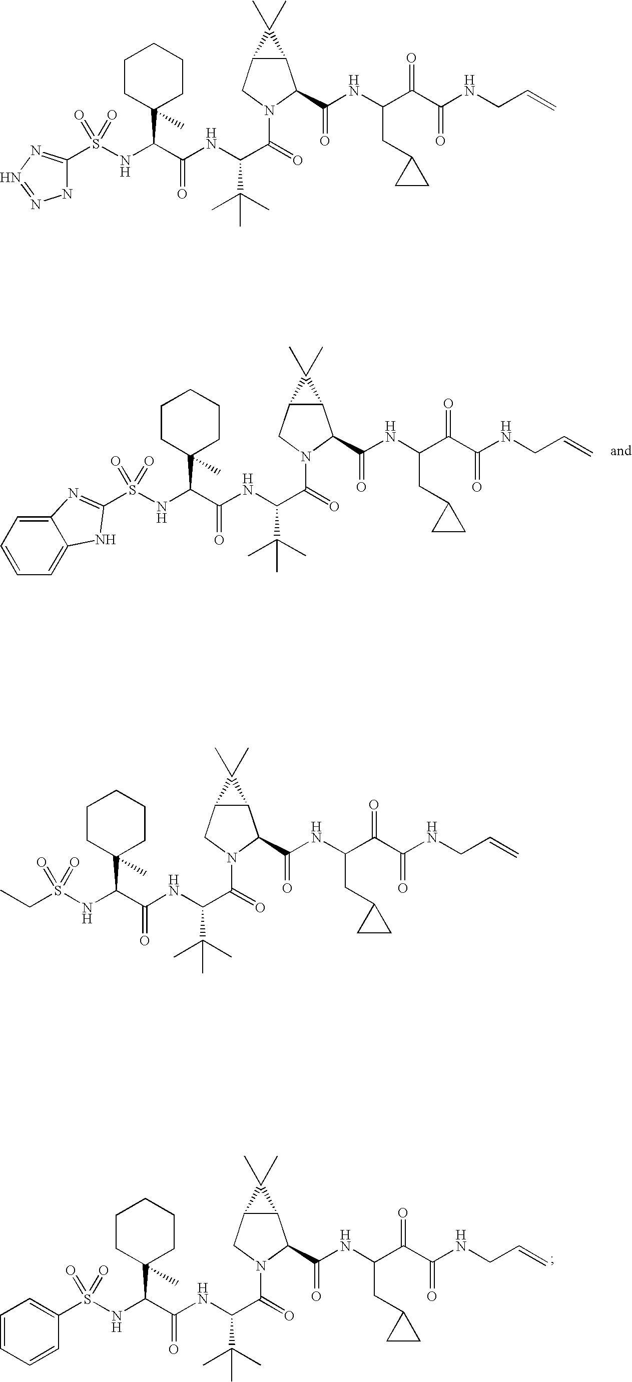
or a pharmaceutically acceptable salt, solvate or ester thereof.

Compounds of formula XVI are disclosed in U.S. patent application Ser. No. 11/064,757 filed Feb. 24, 2005. The preparation of these compounds is disclosed in the experimental section of this application set forth hereinbelow.

Non-limiting examples of certain compounds disclosed in U.S. patent application Ser. No. 11/064,757 are: 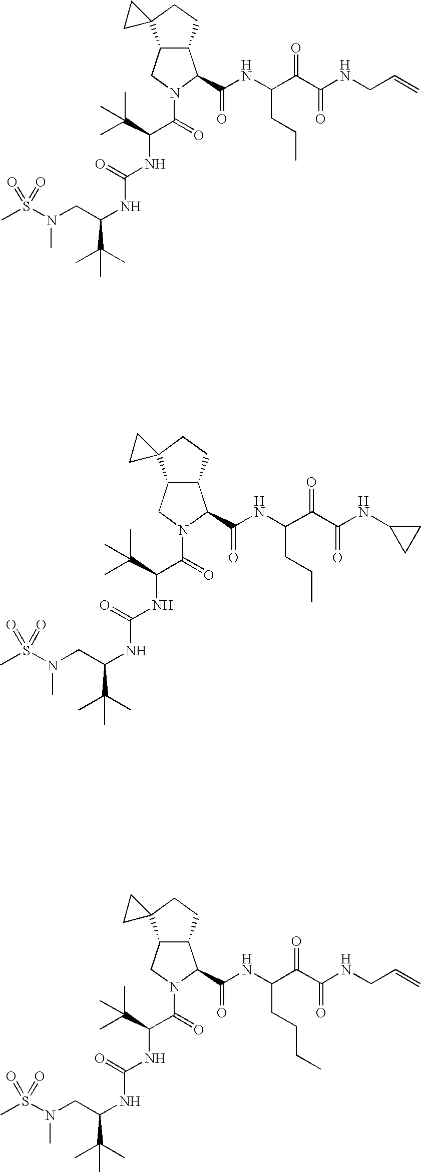 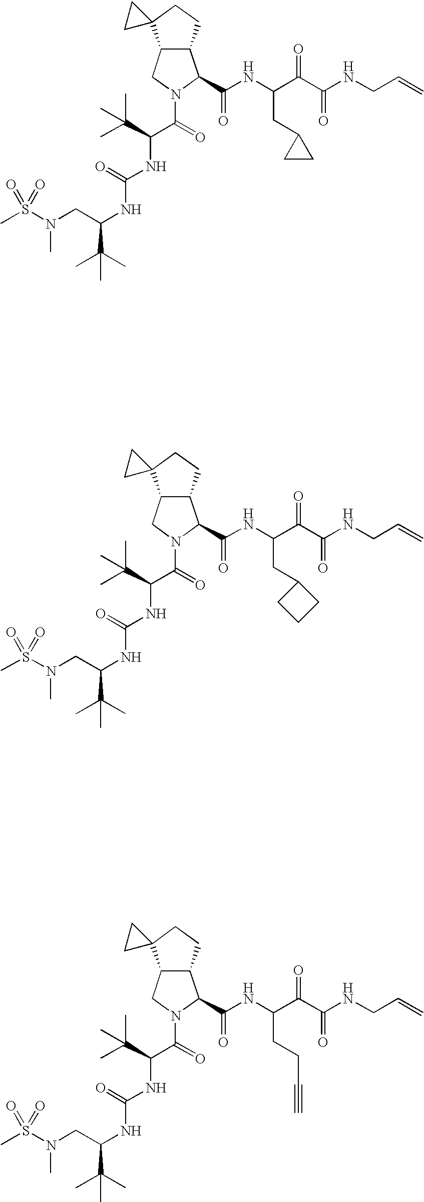 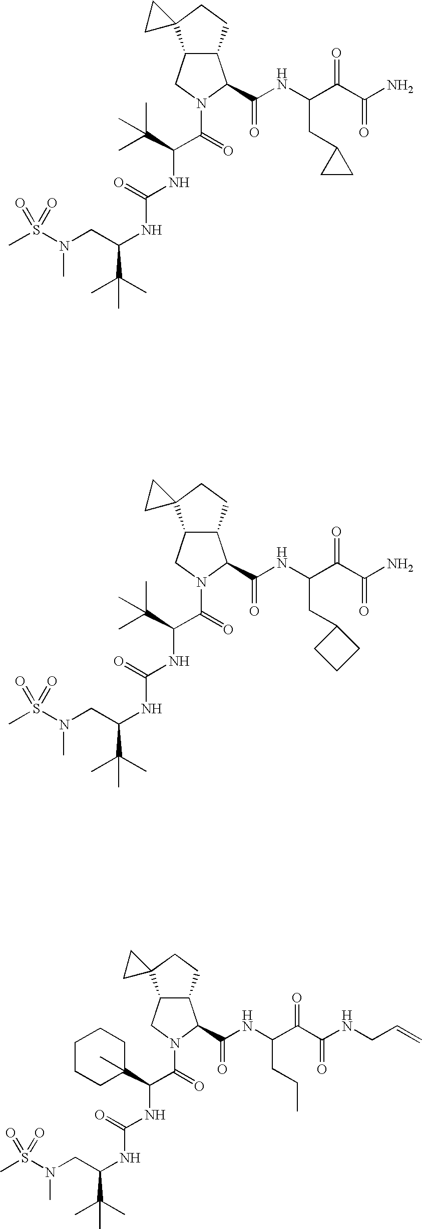 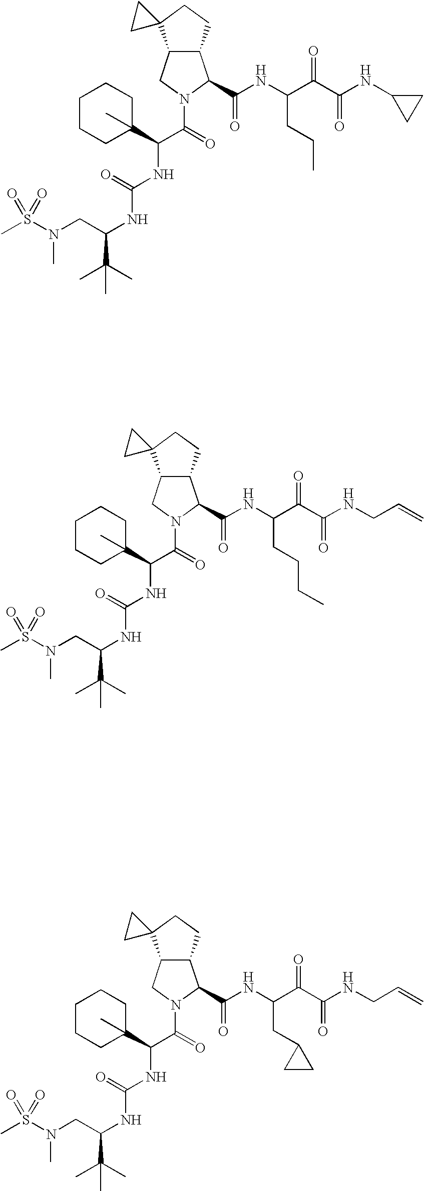 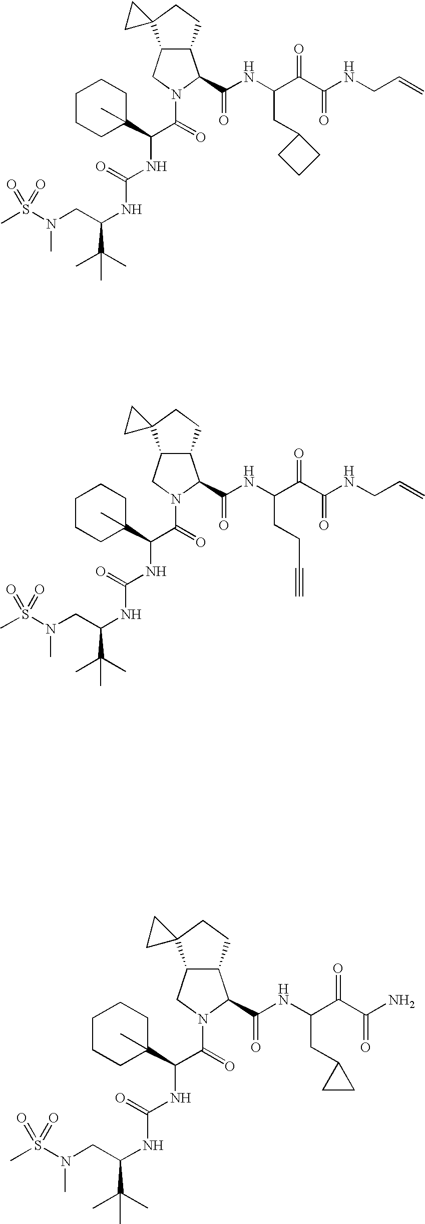 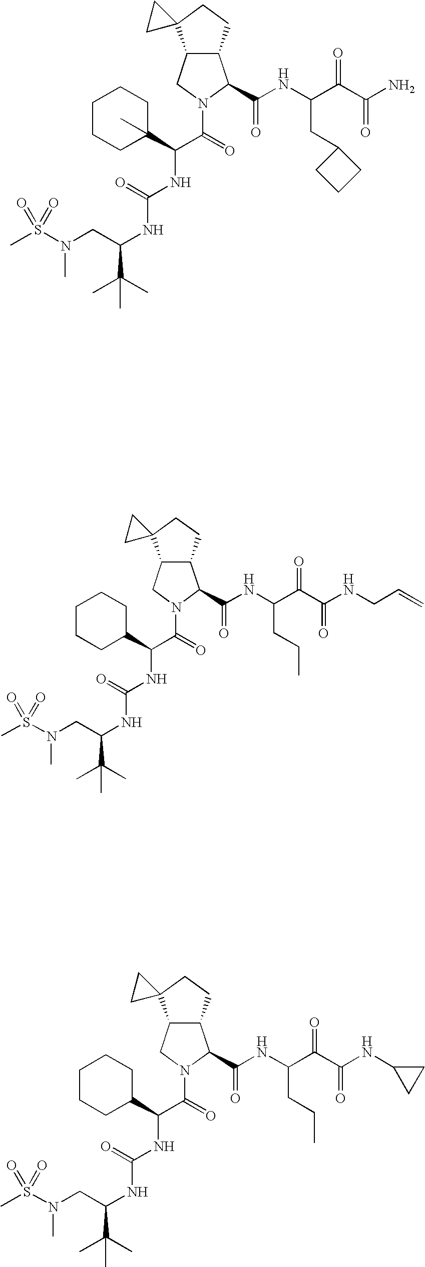 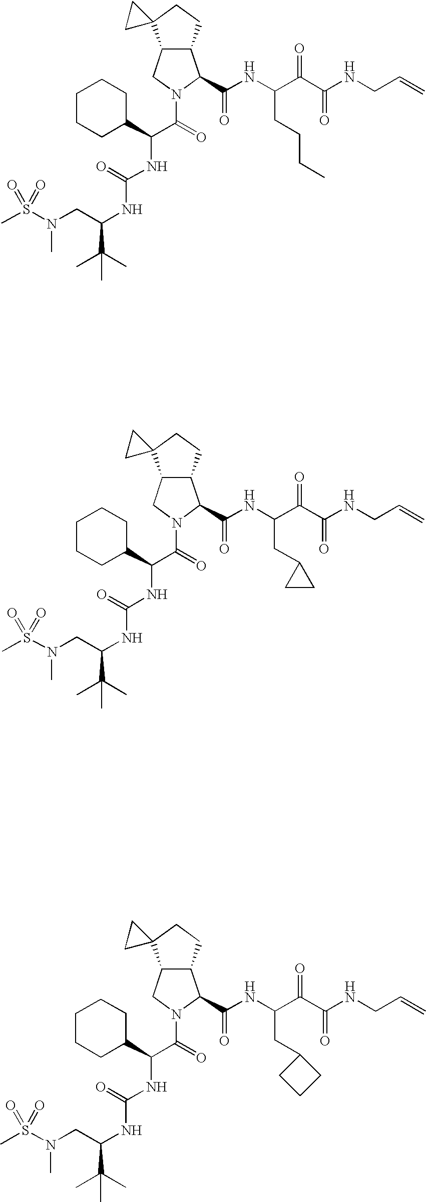 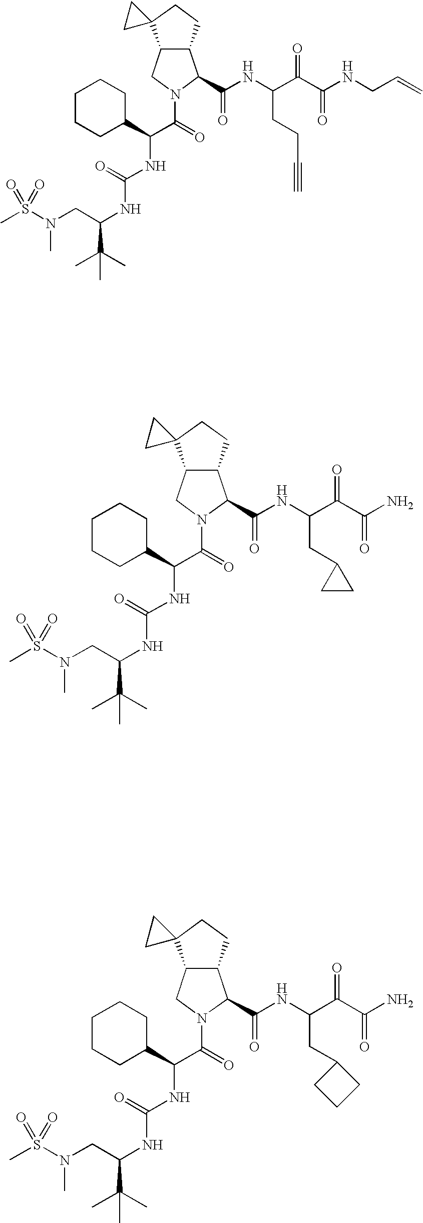 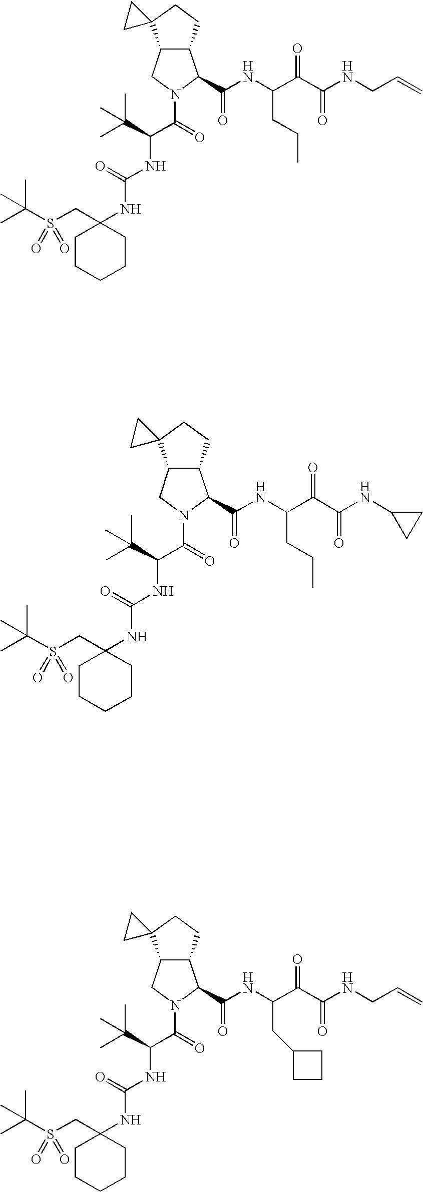 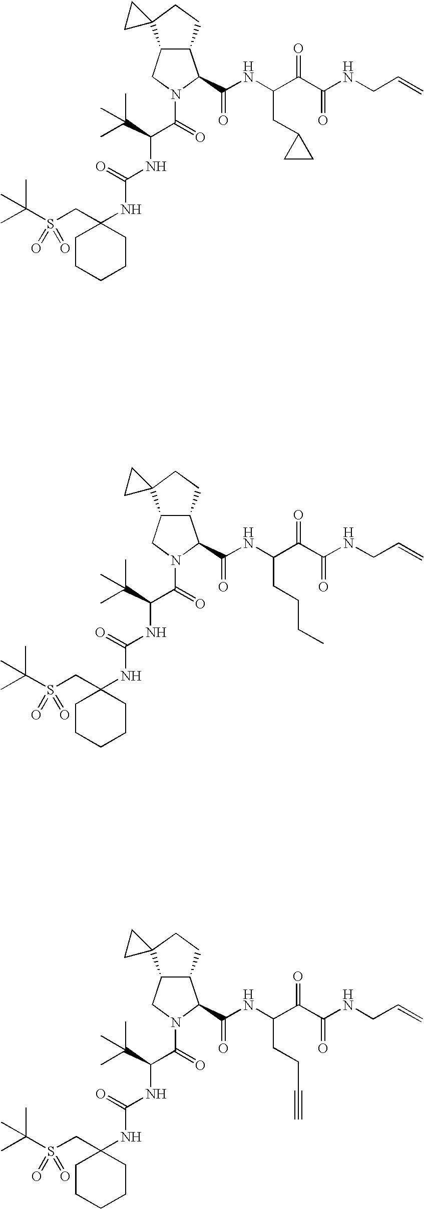 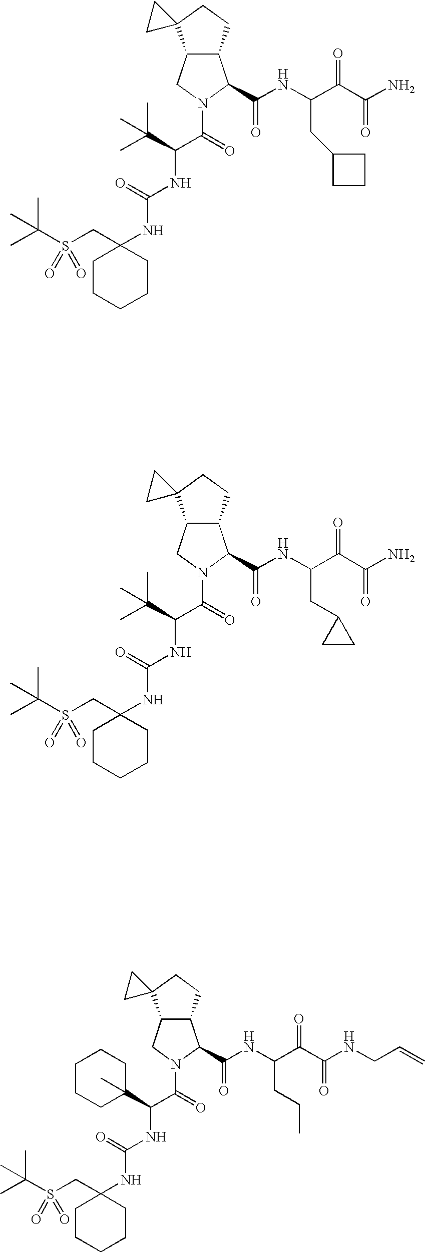 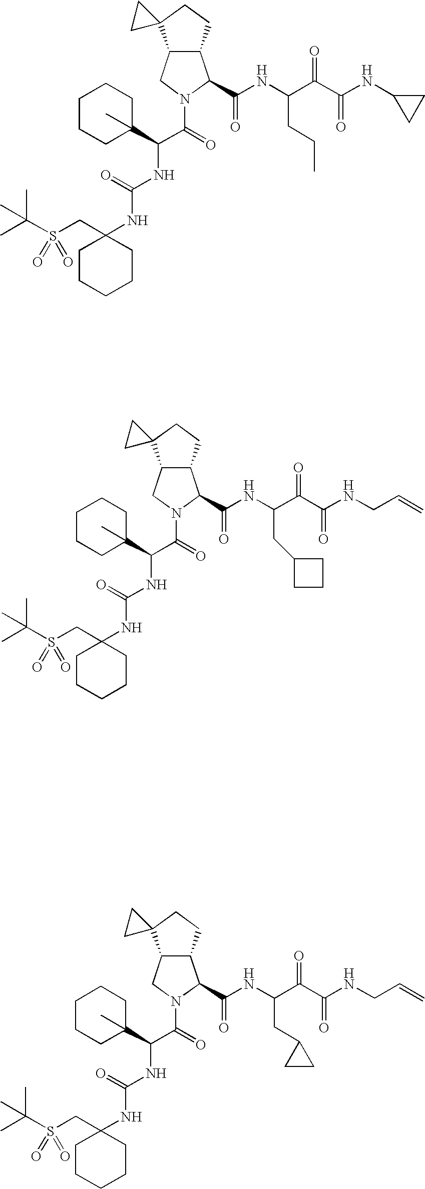 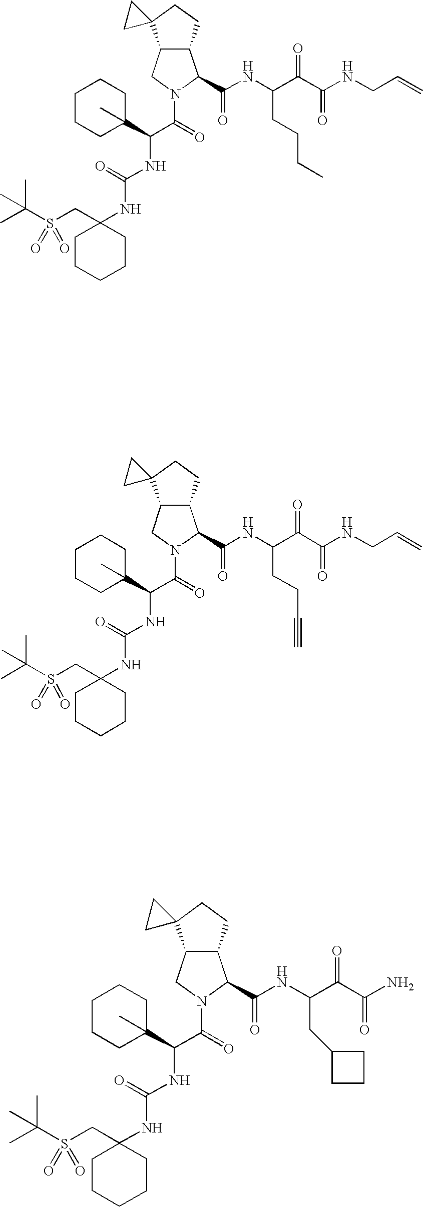 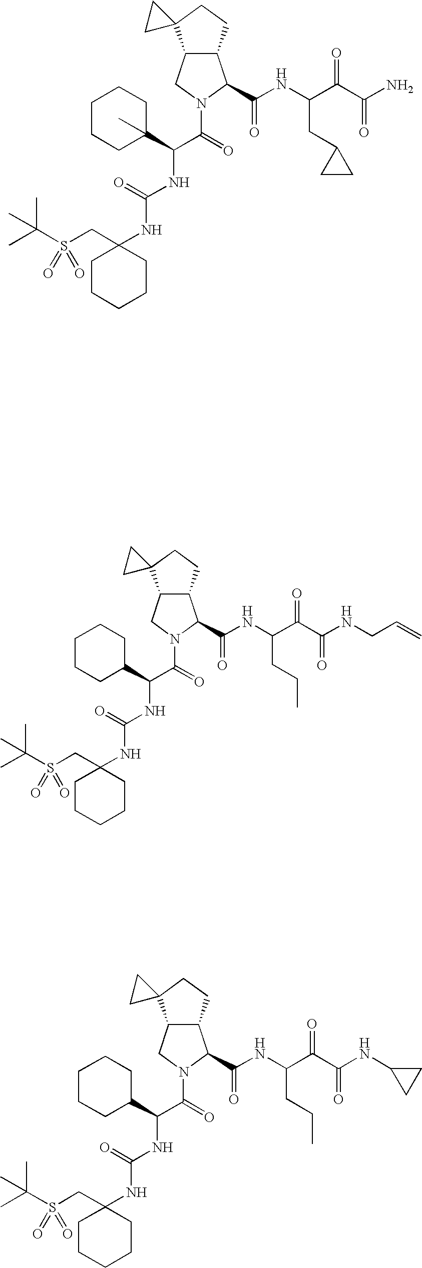 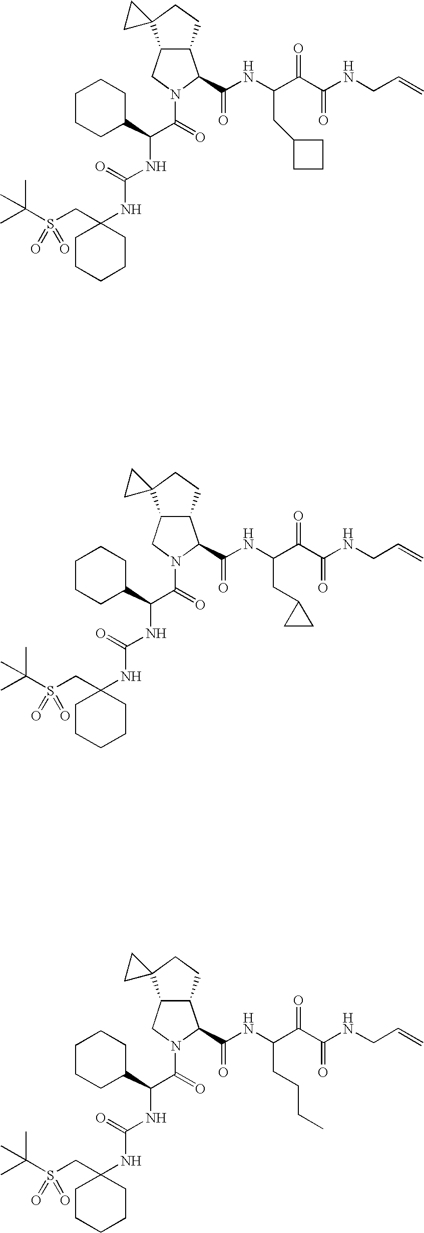 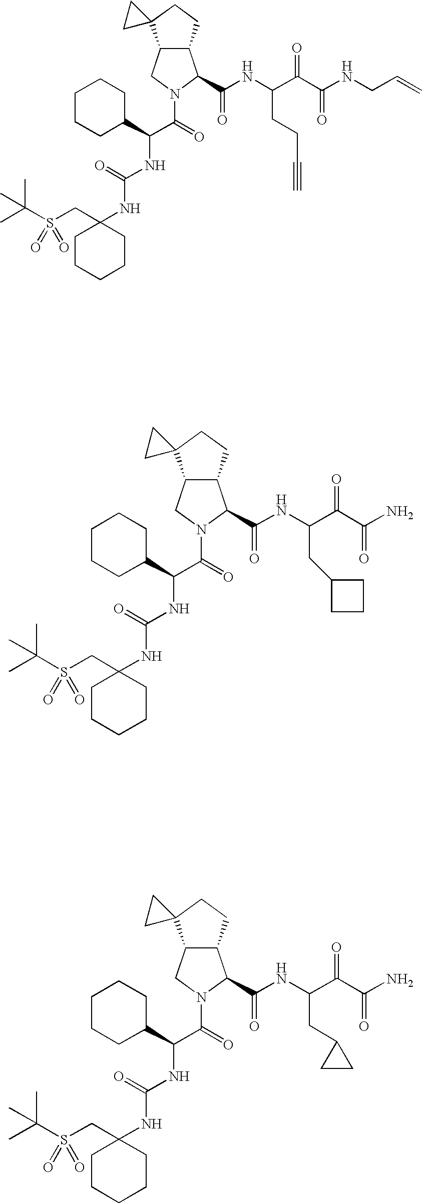 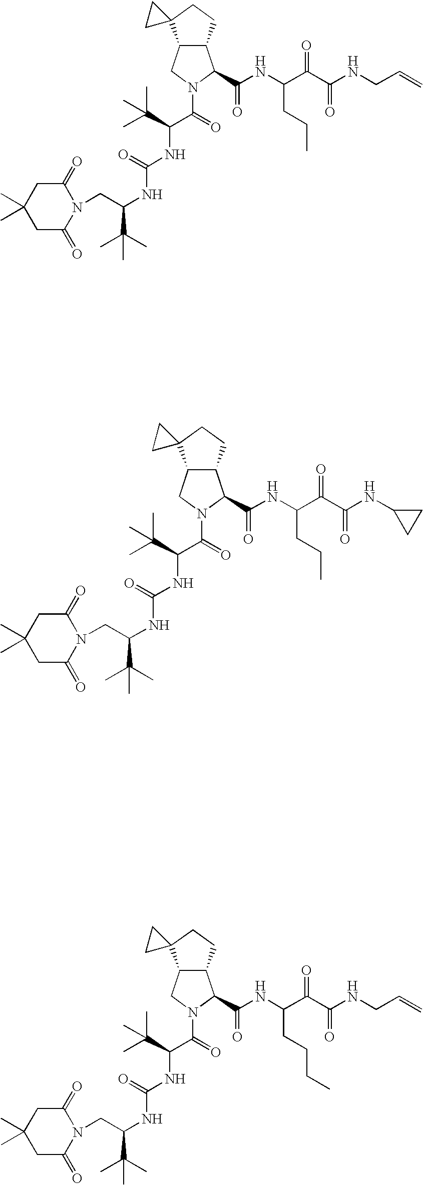 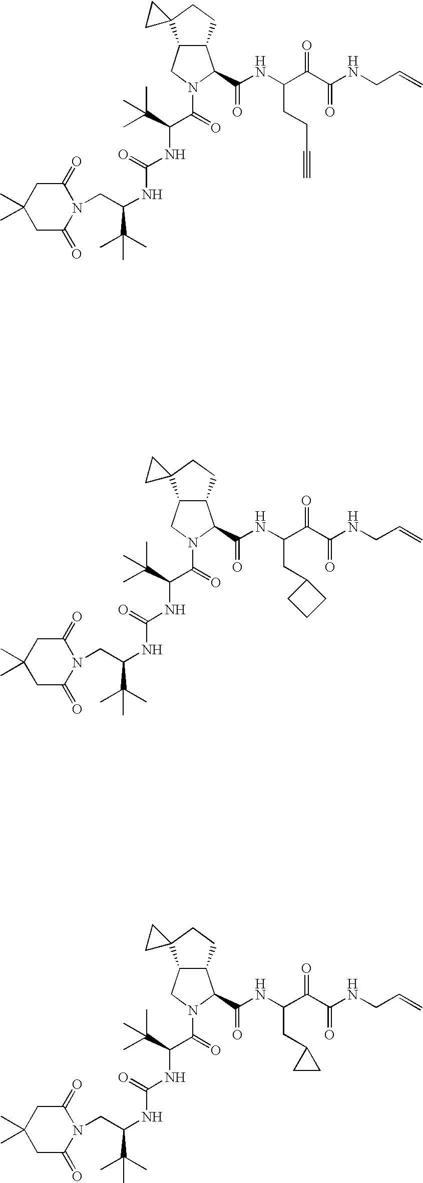 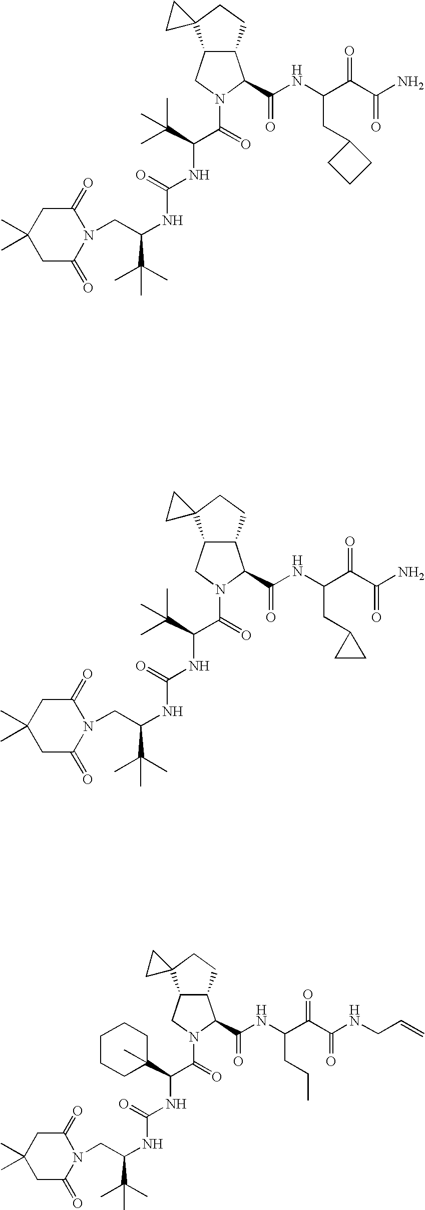 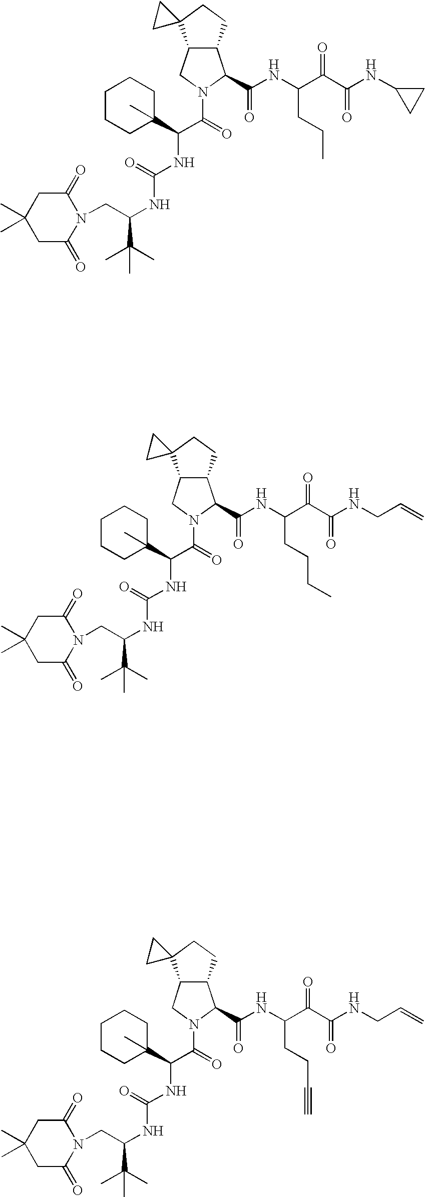 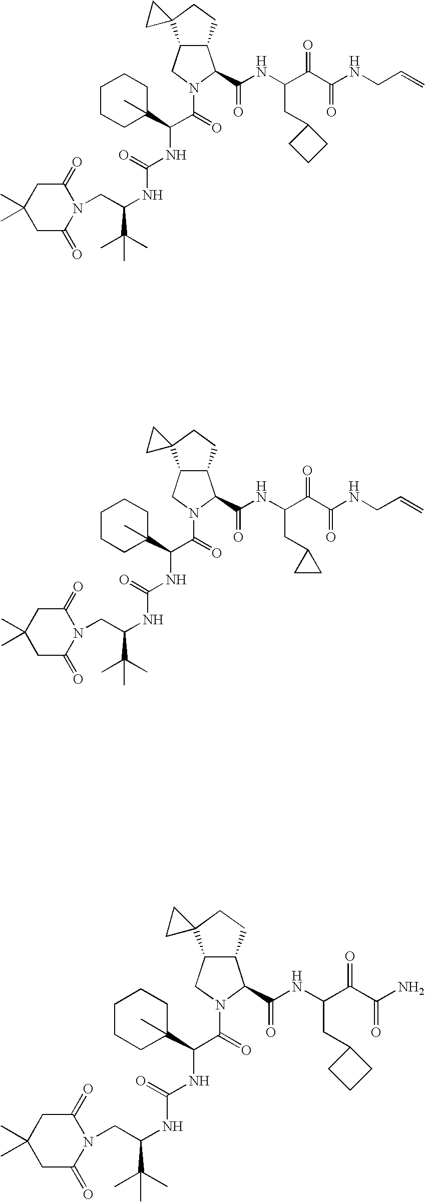 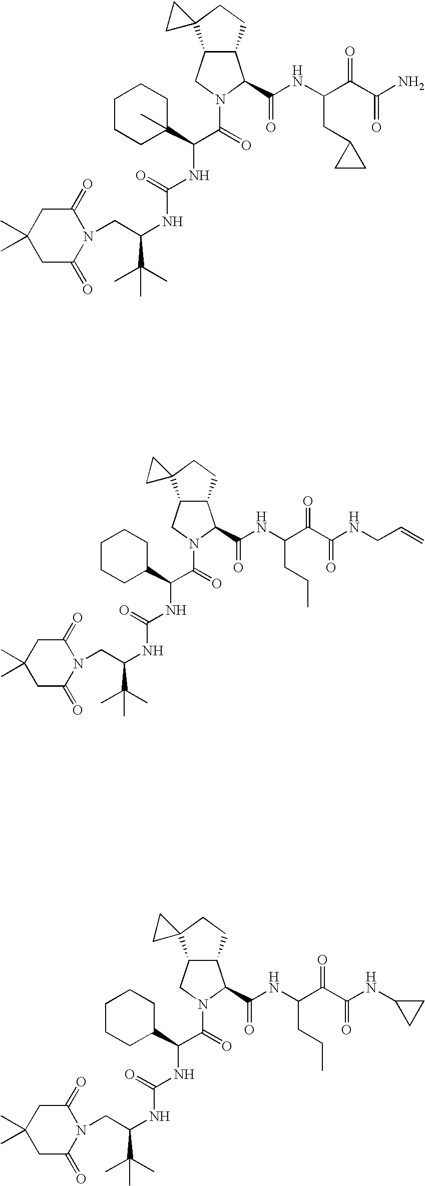 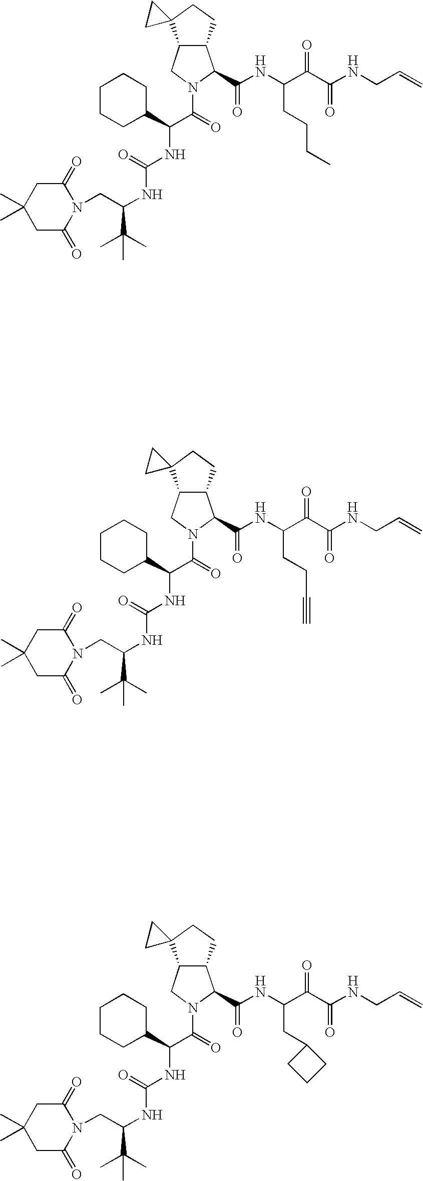 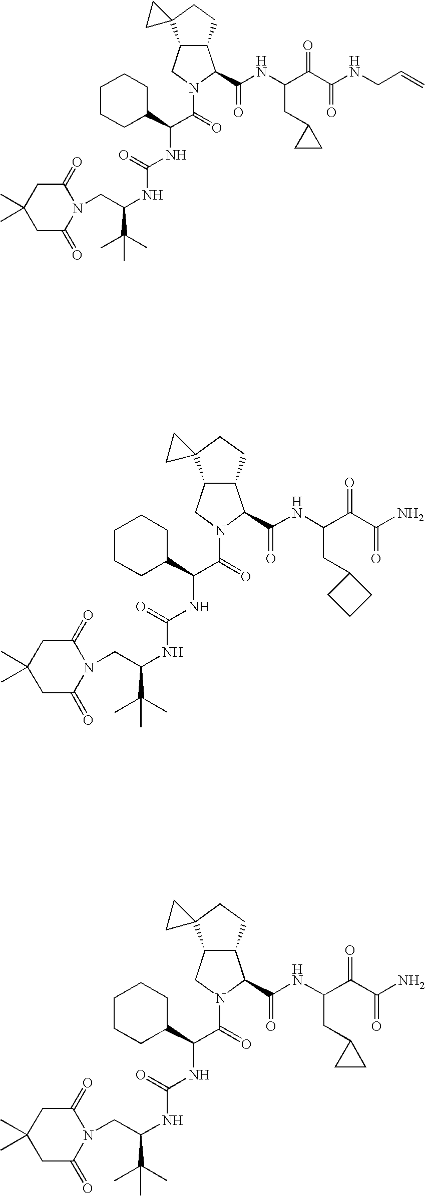 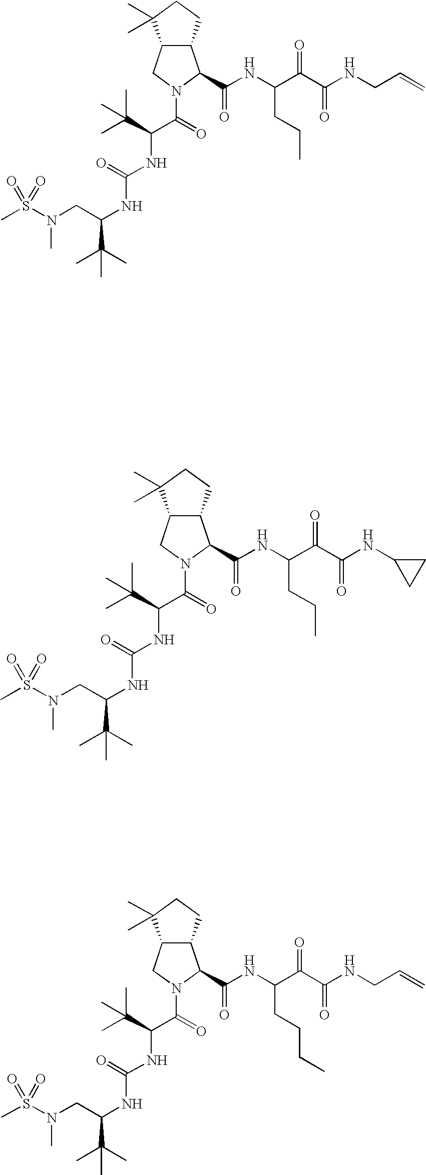 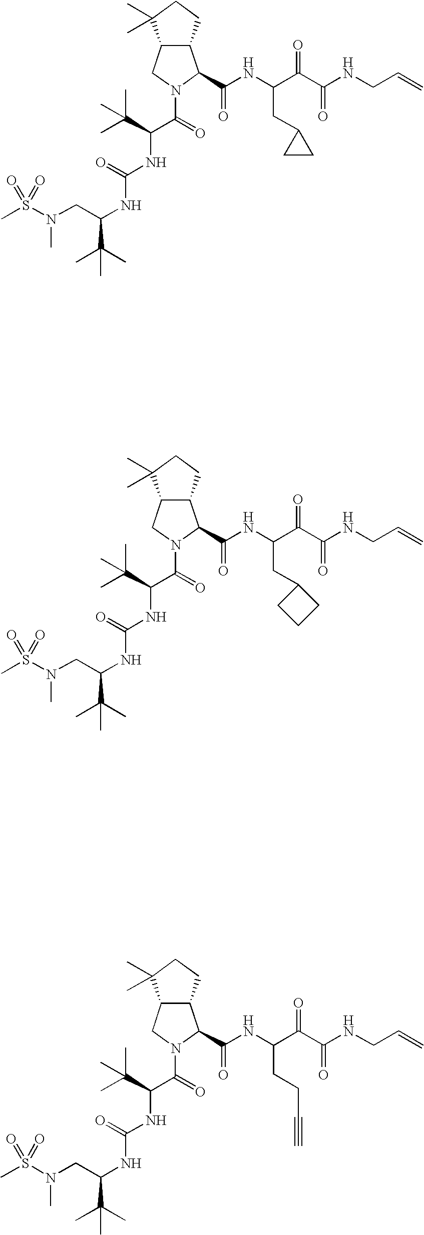 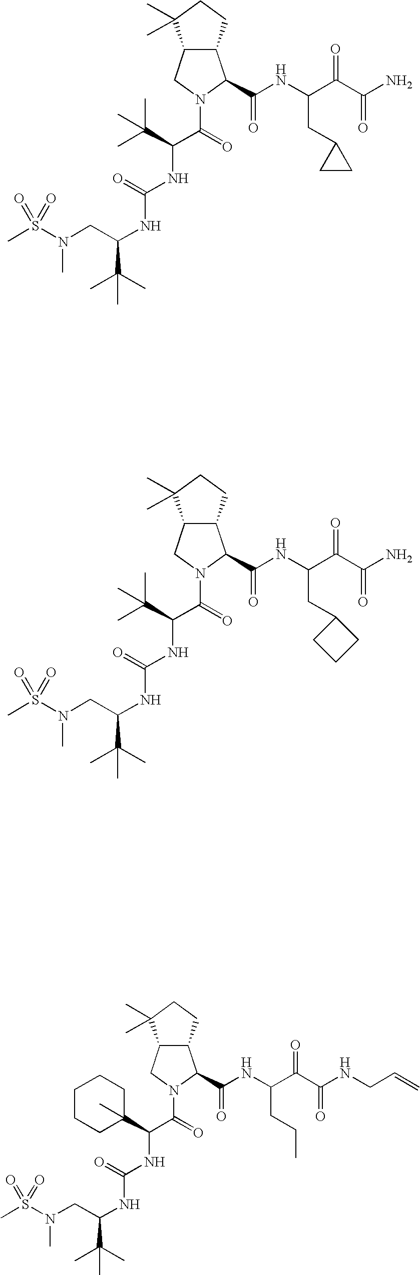 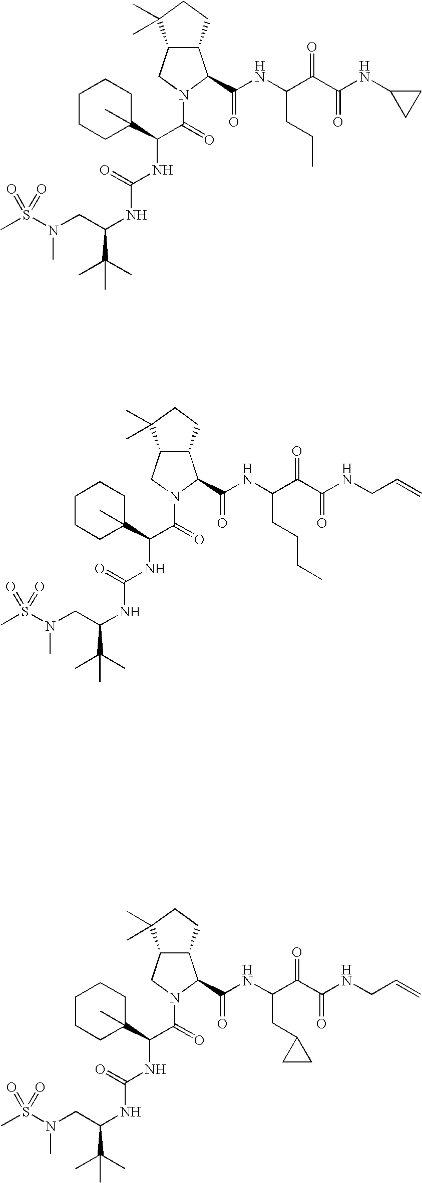 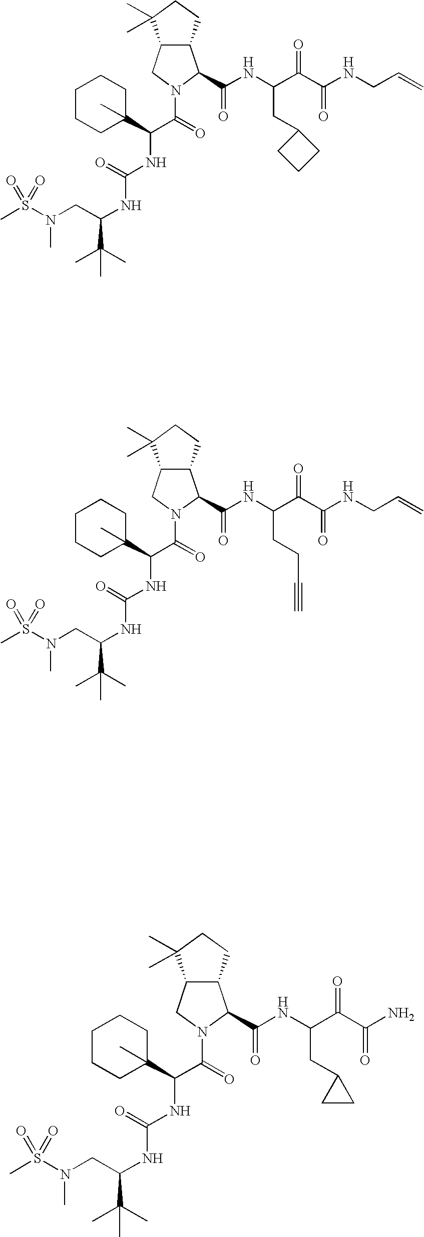 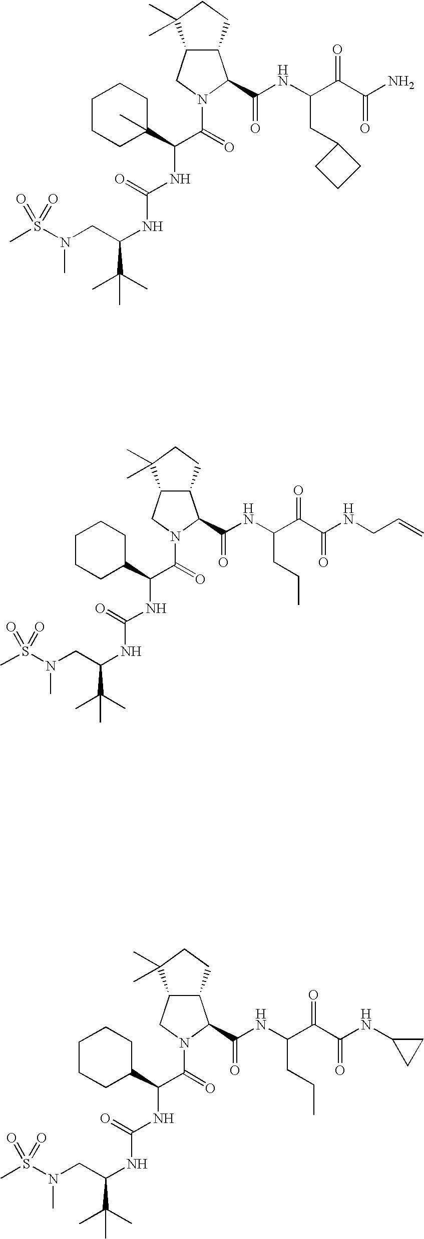 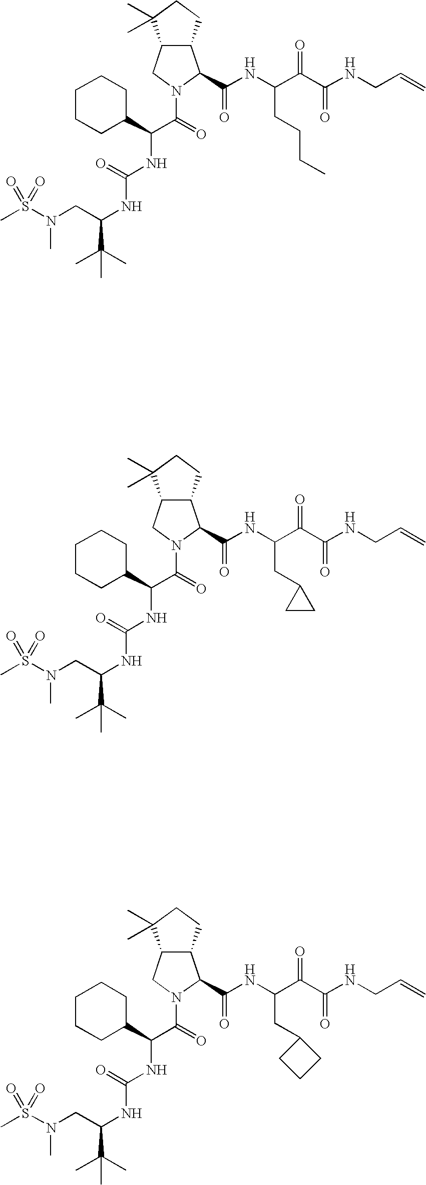 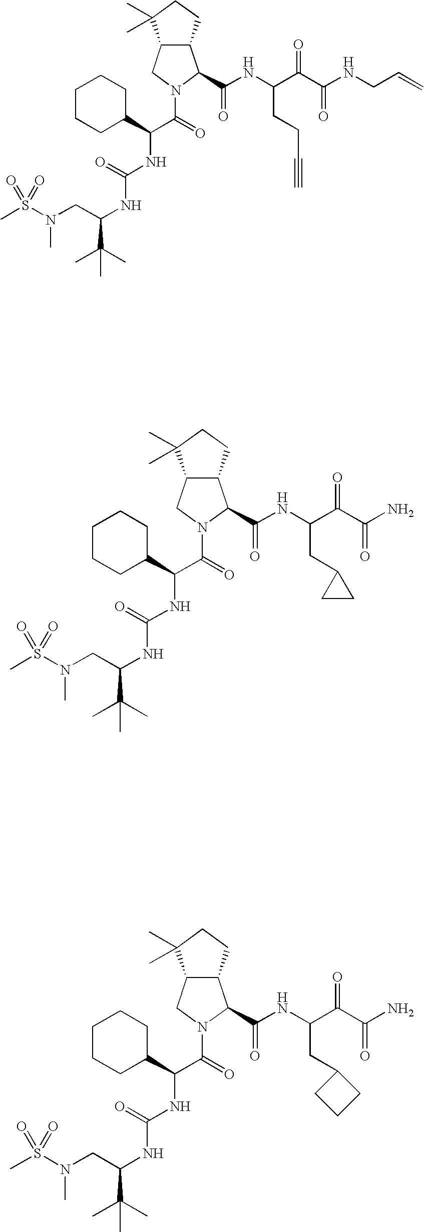 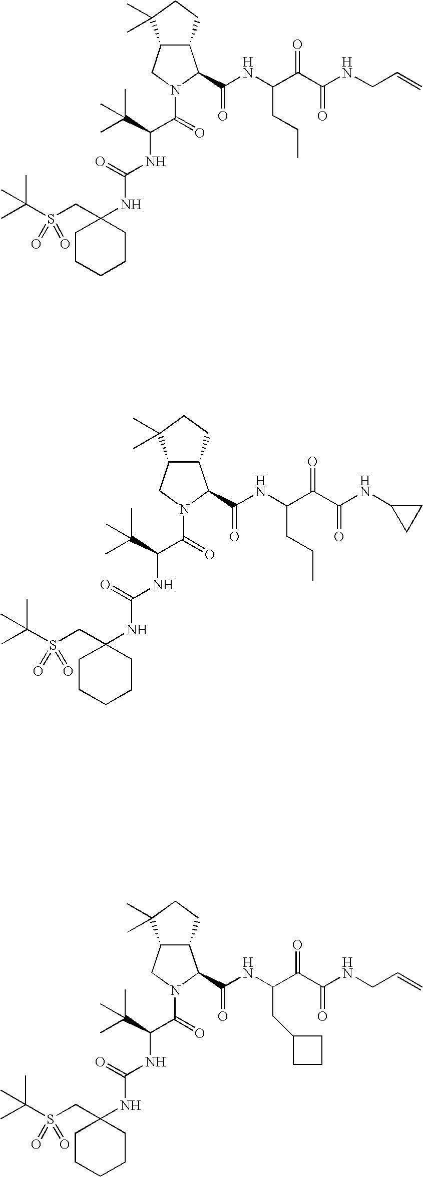 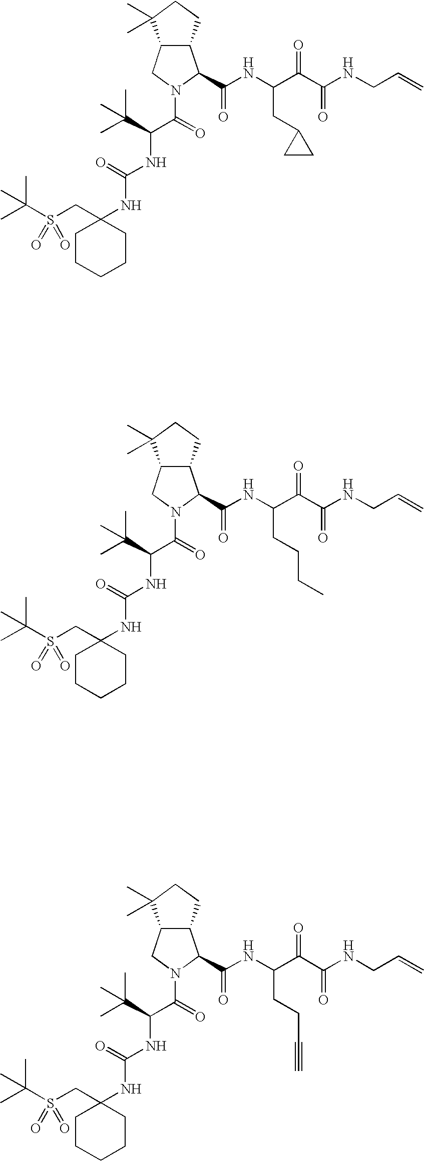 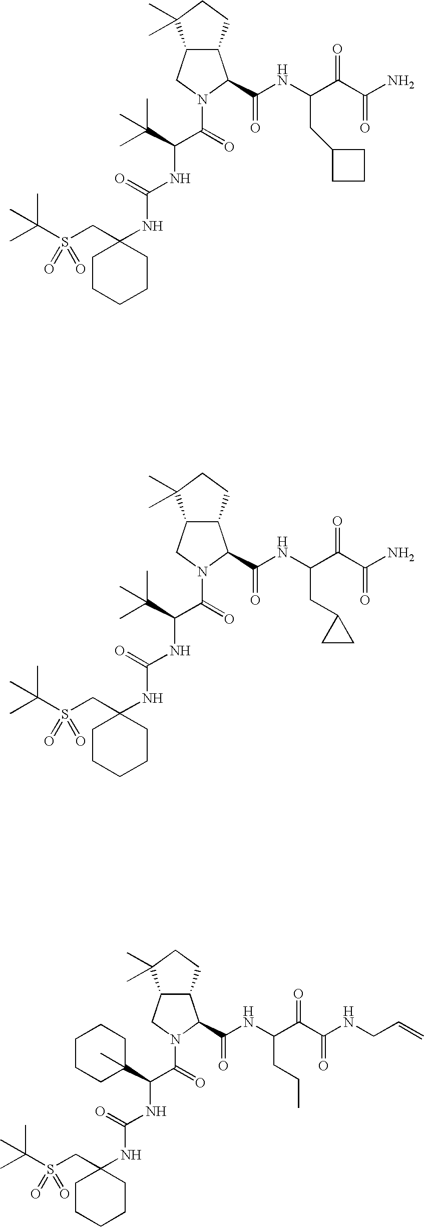 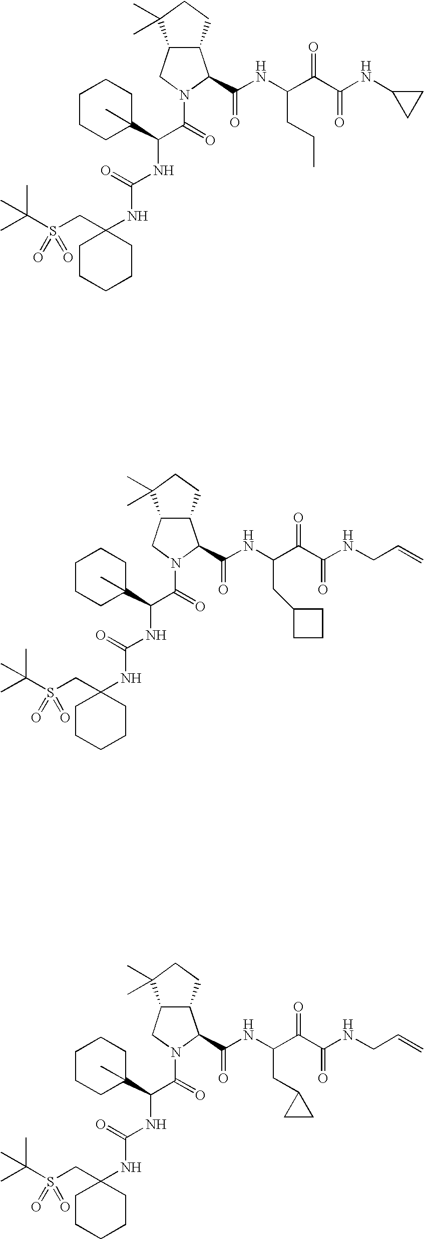 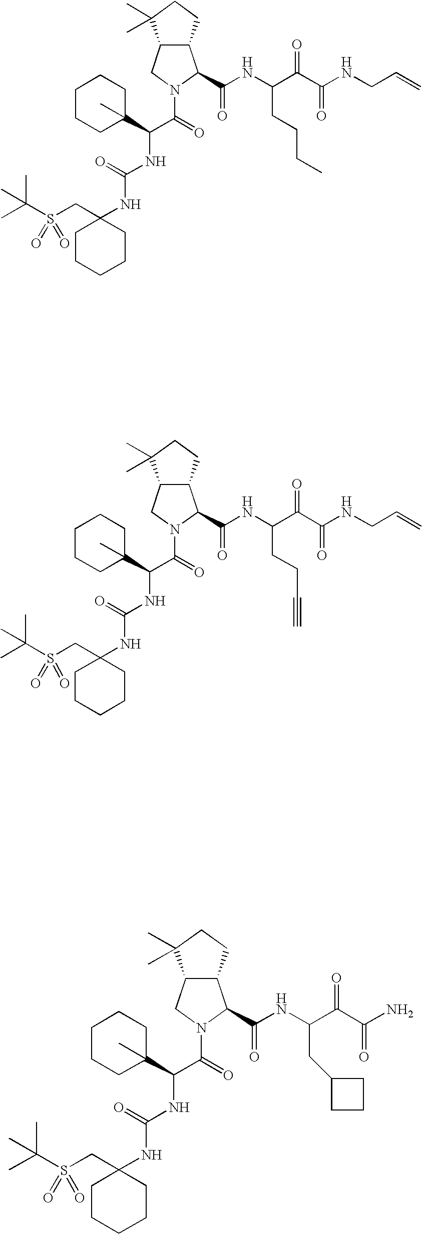 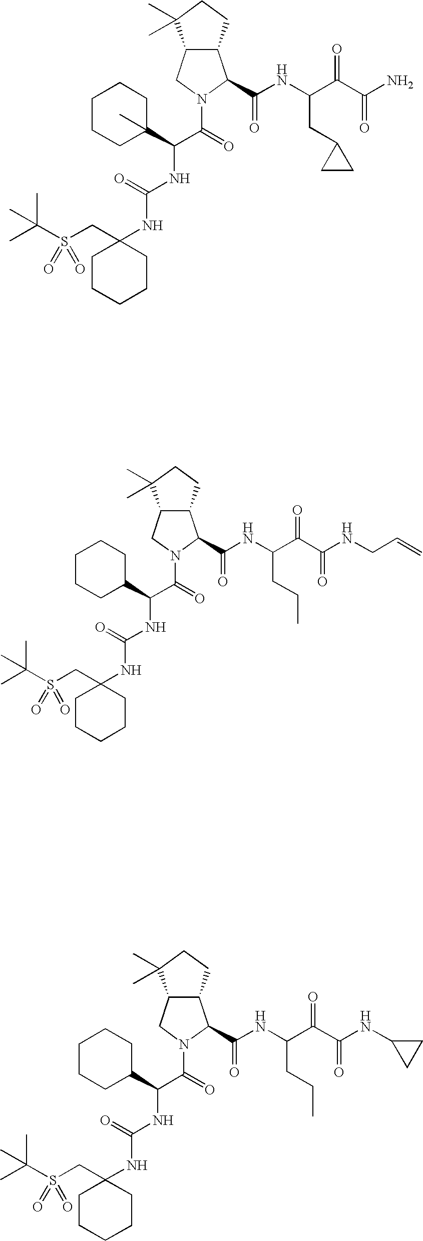 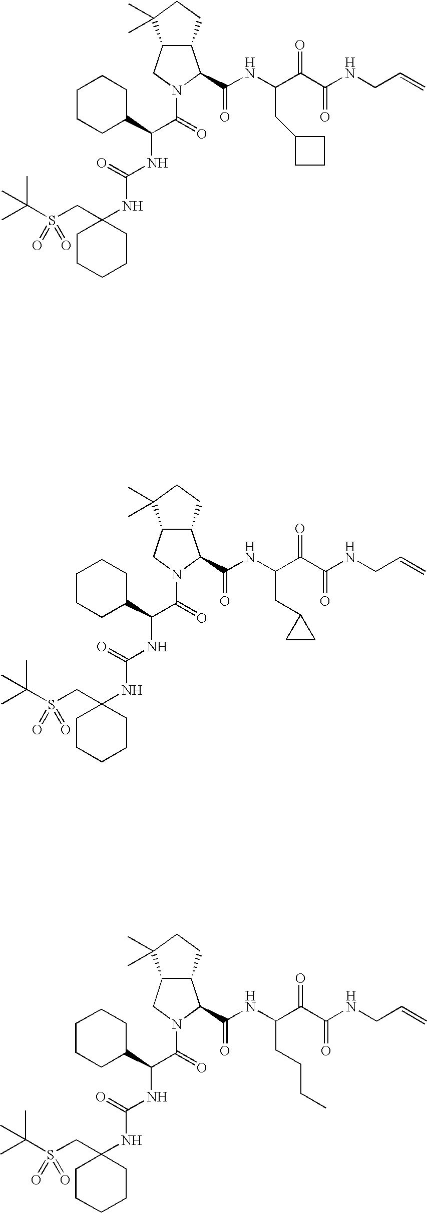 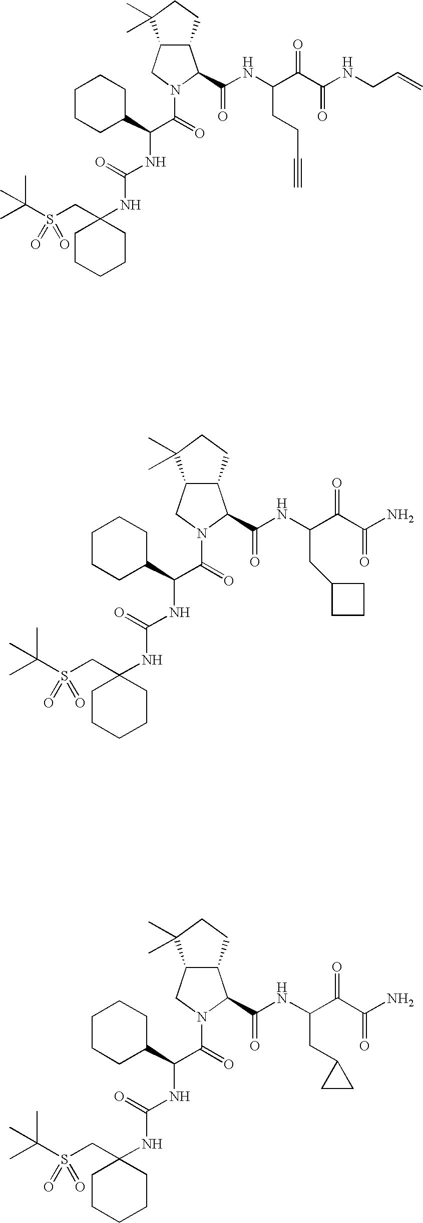 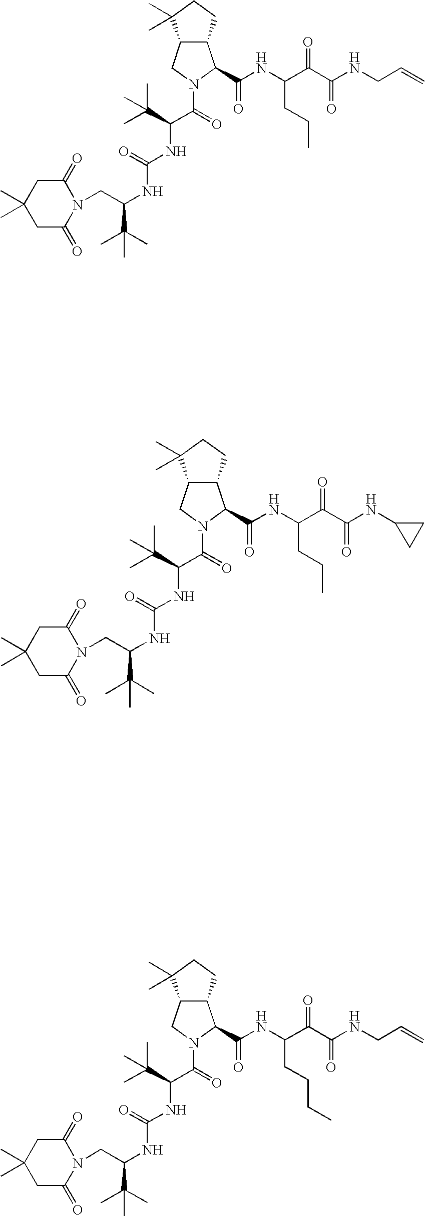 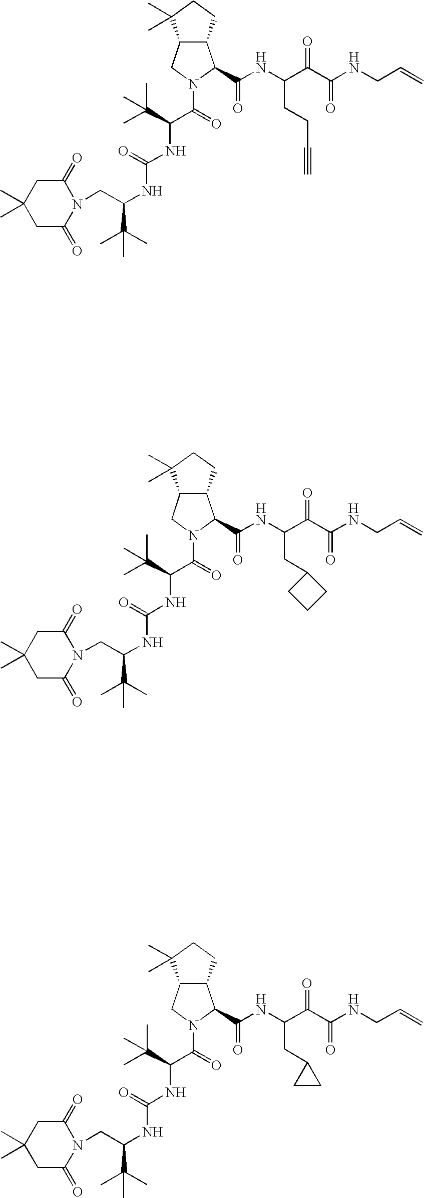 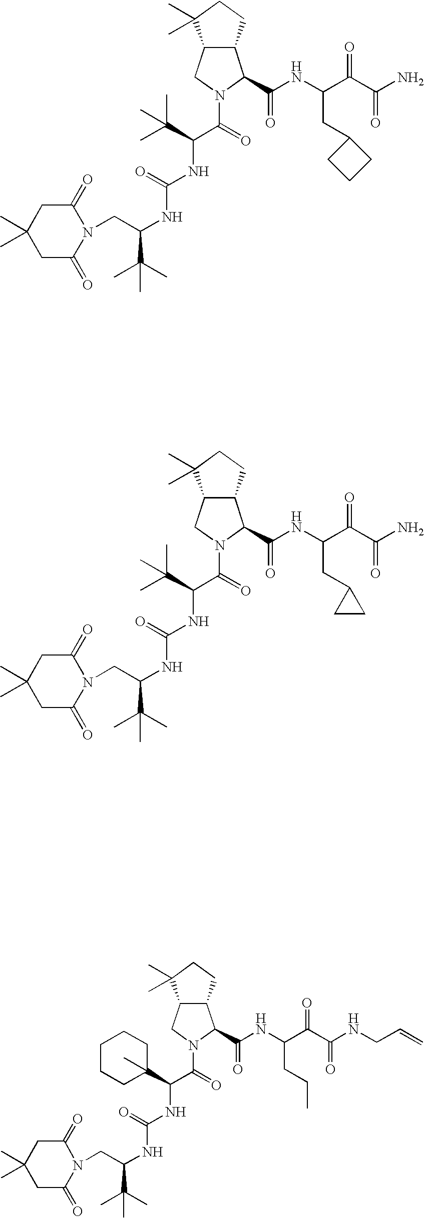 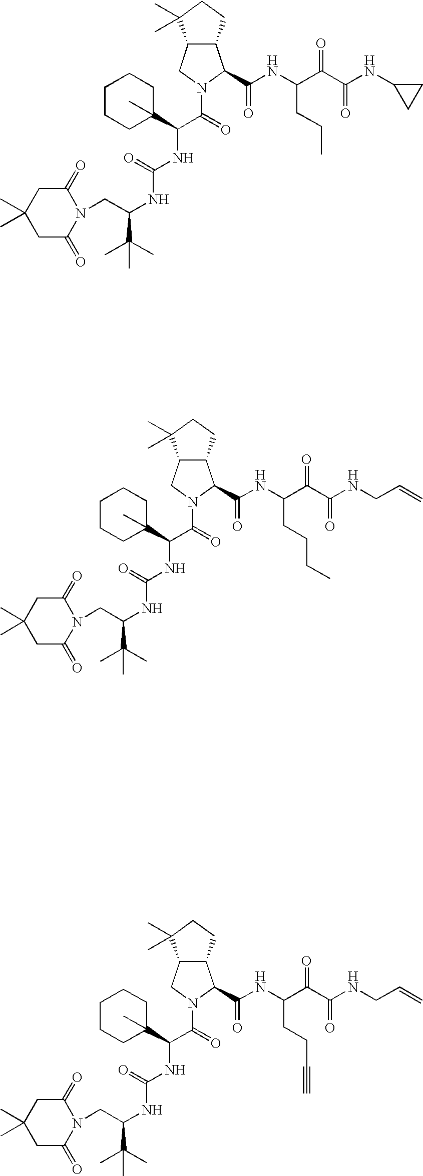 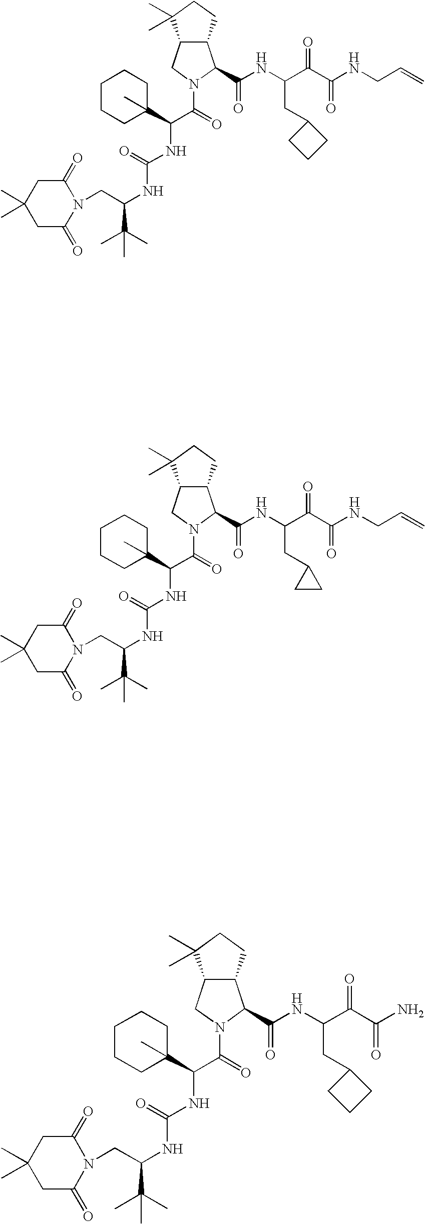 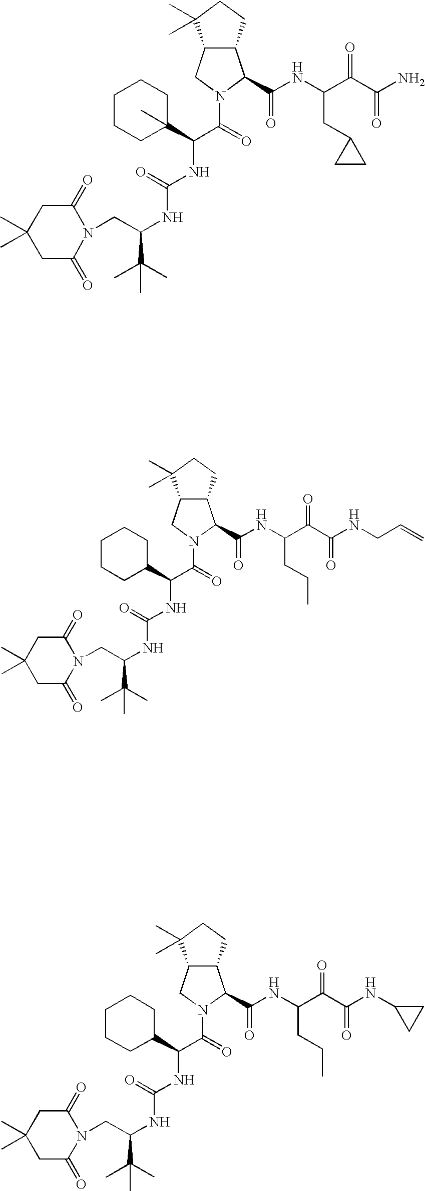 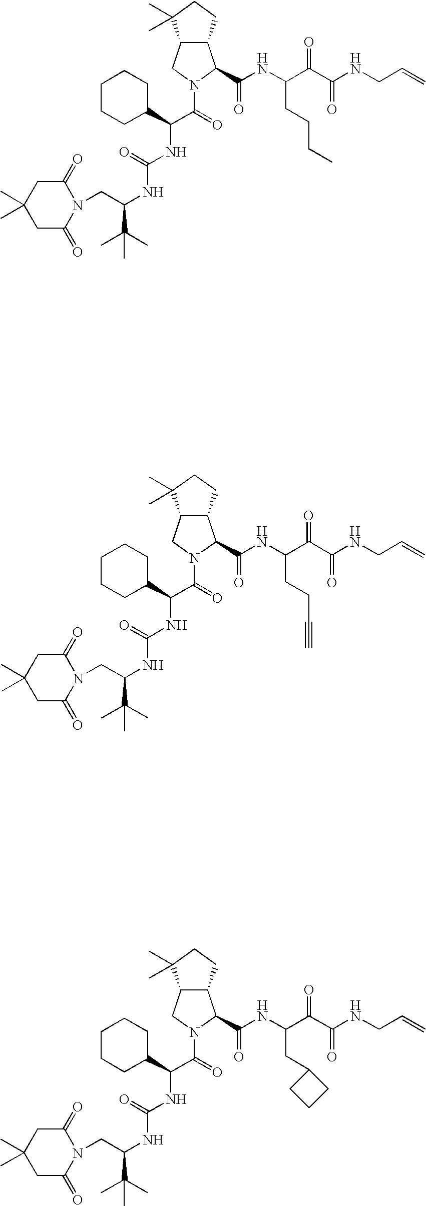 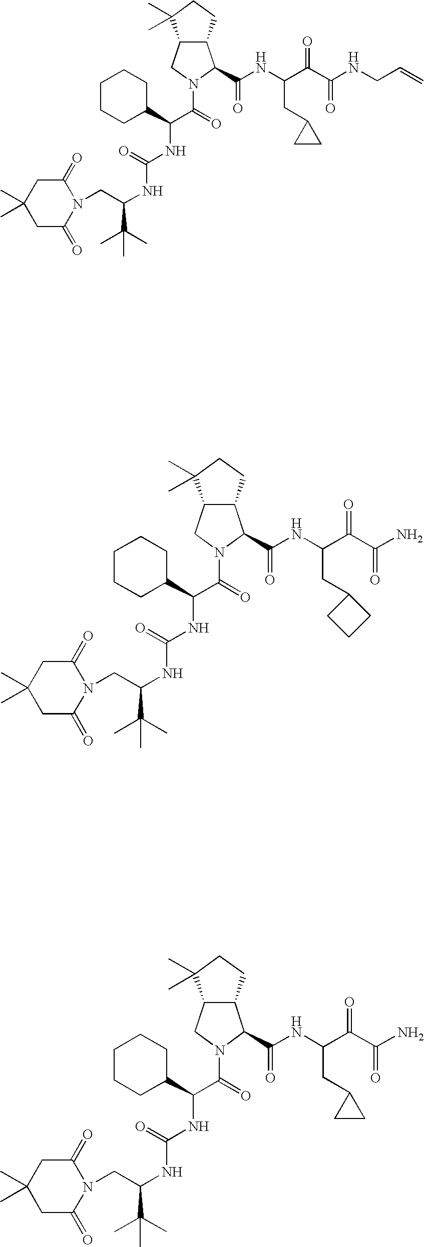 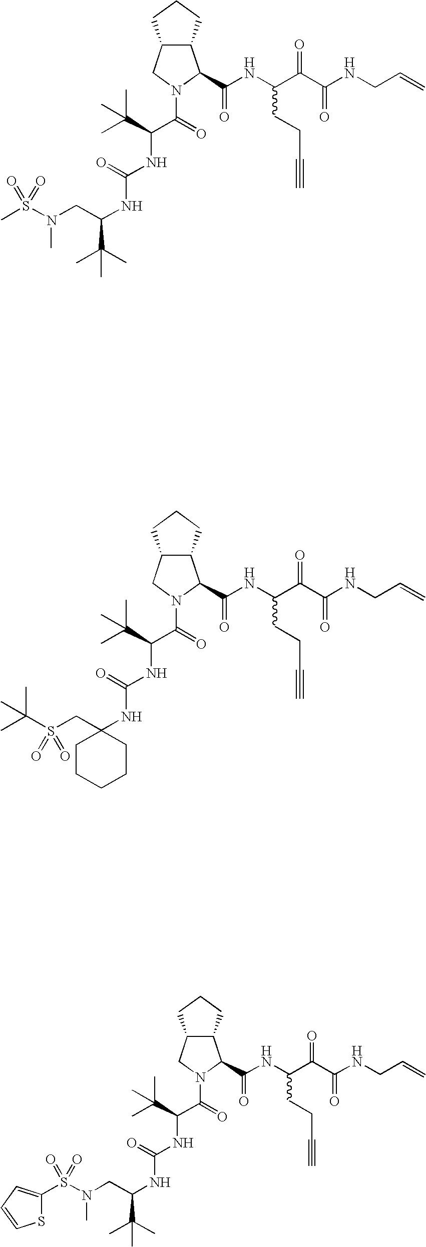 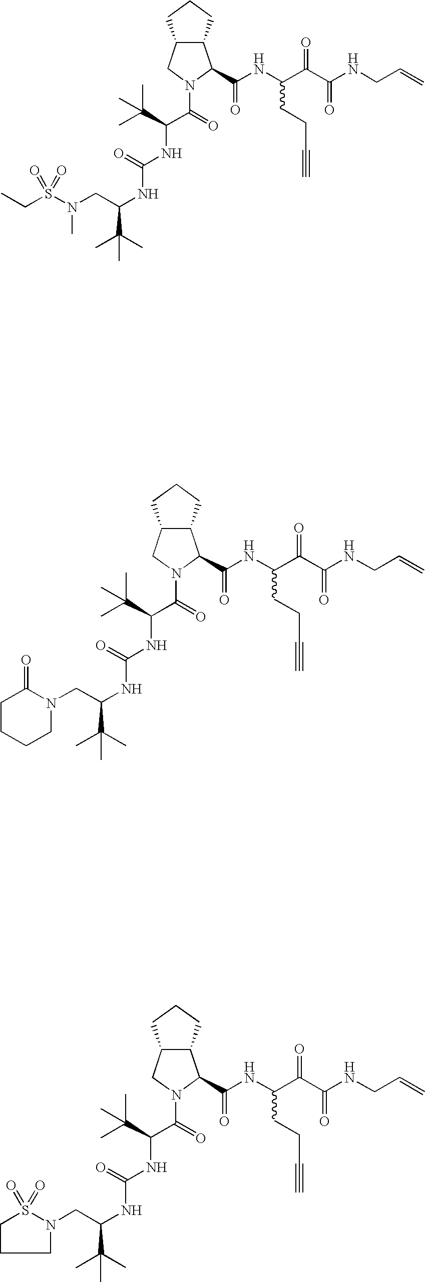 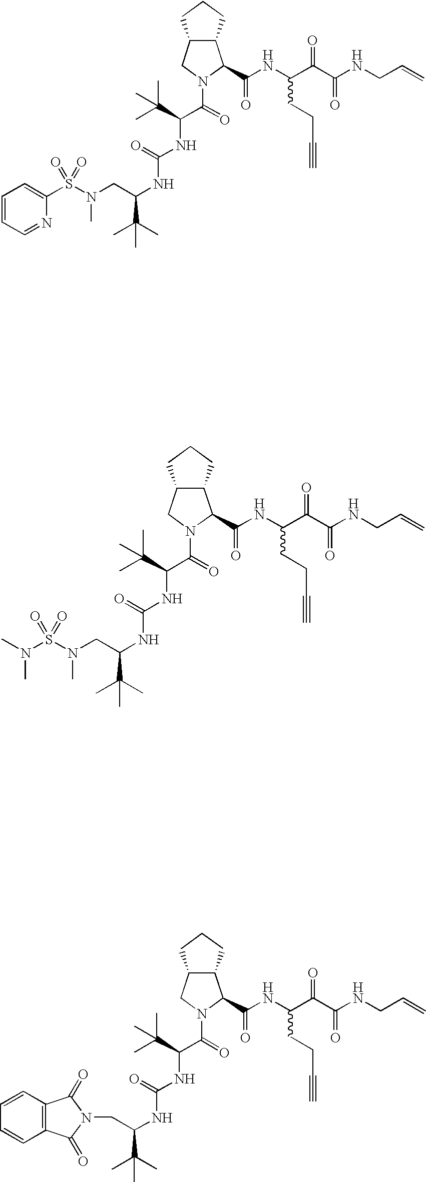 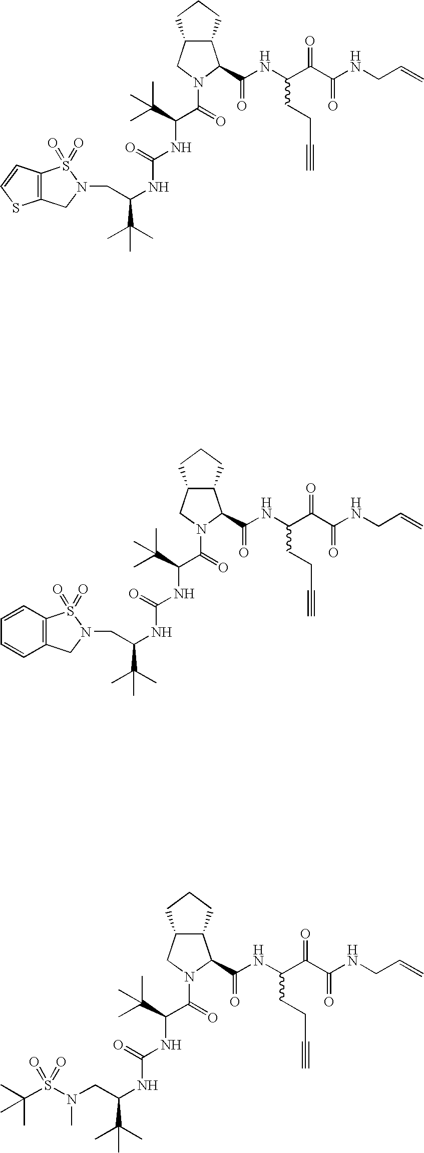 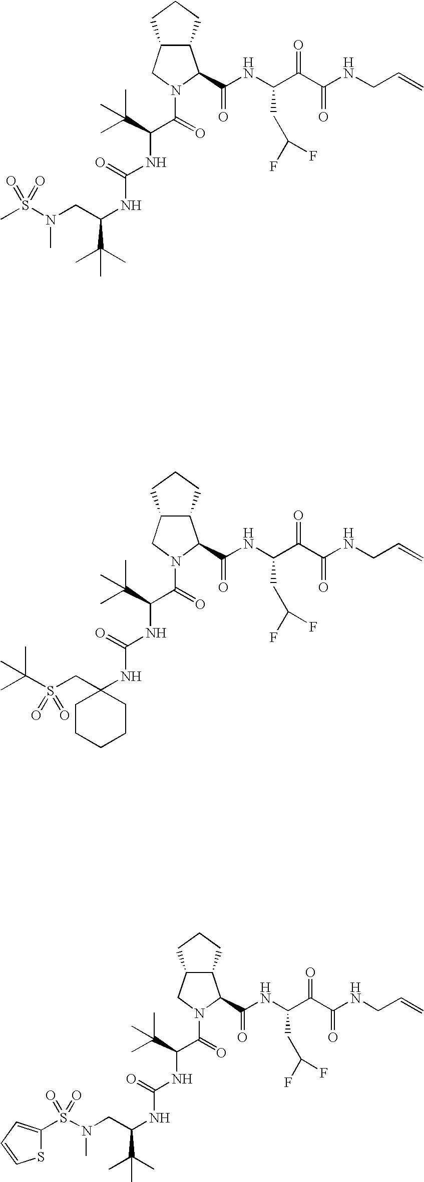 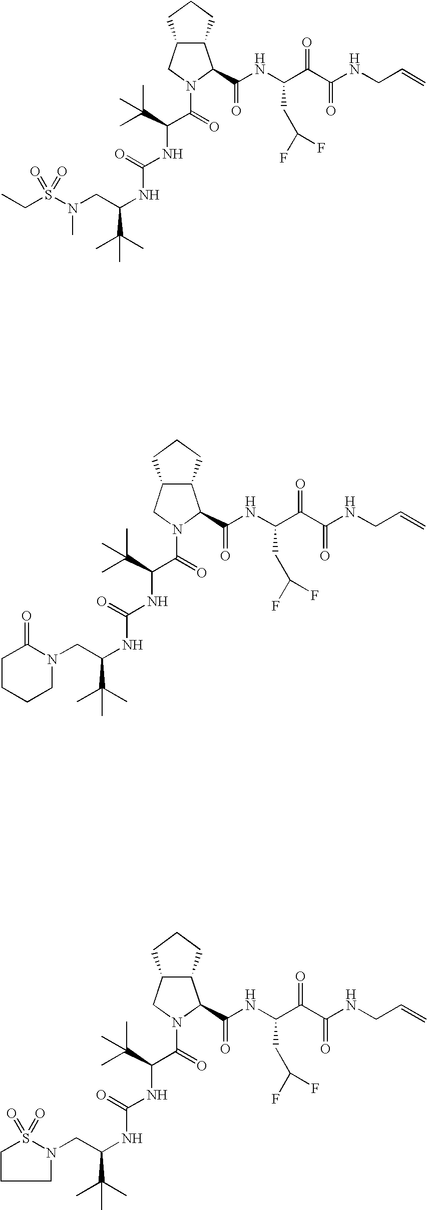 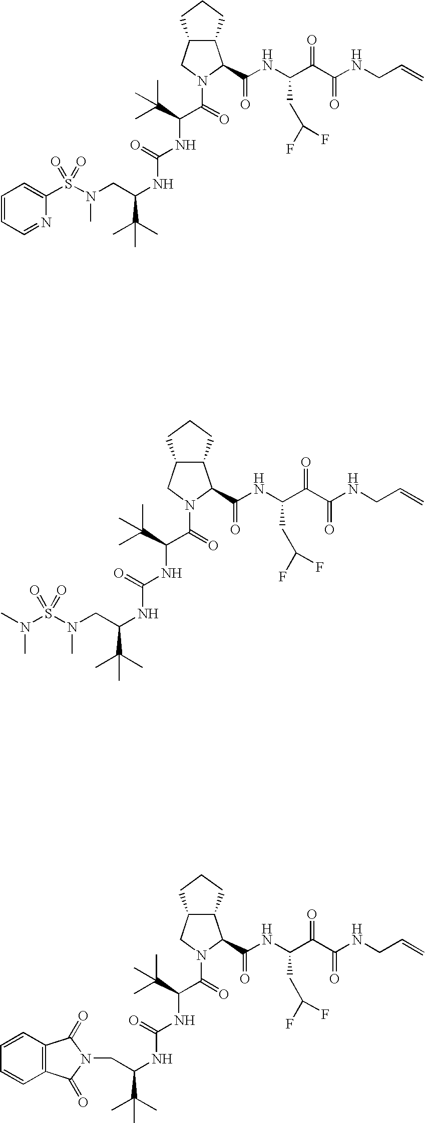 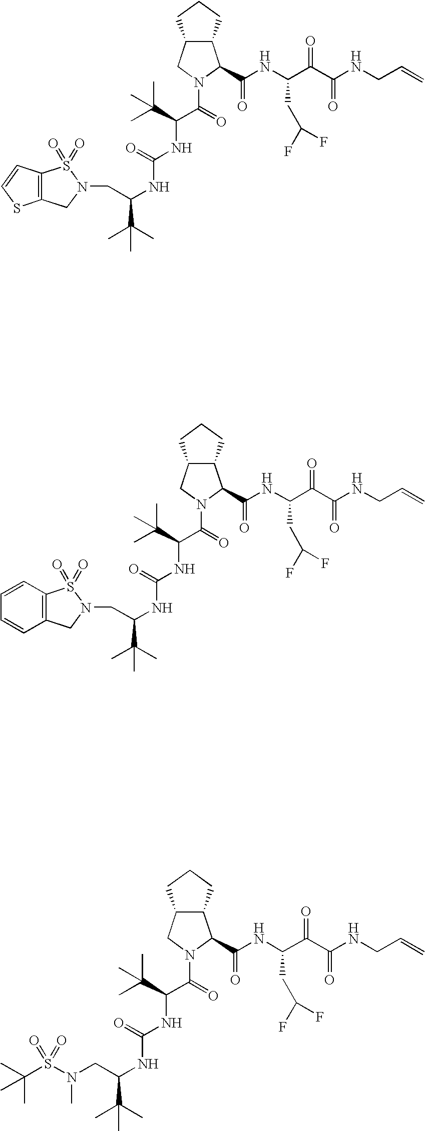 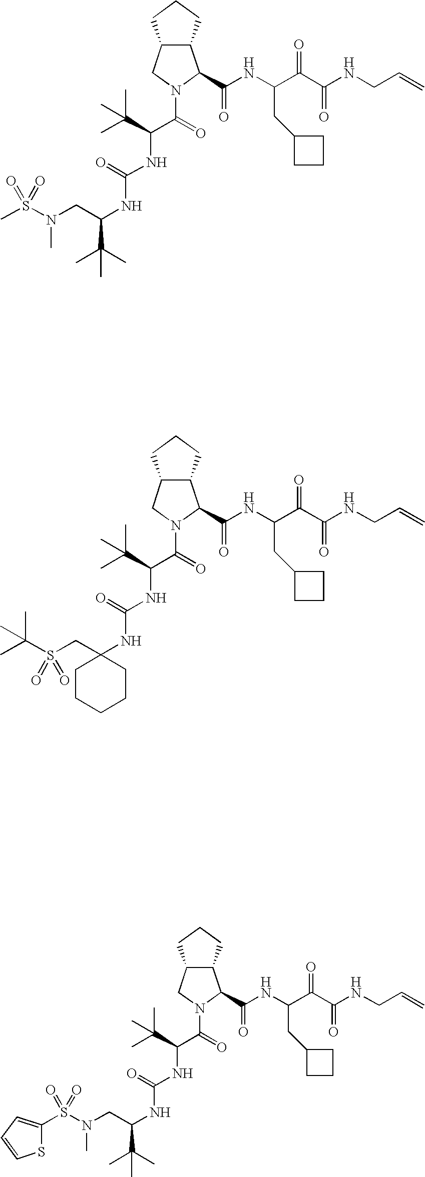 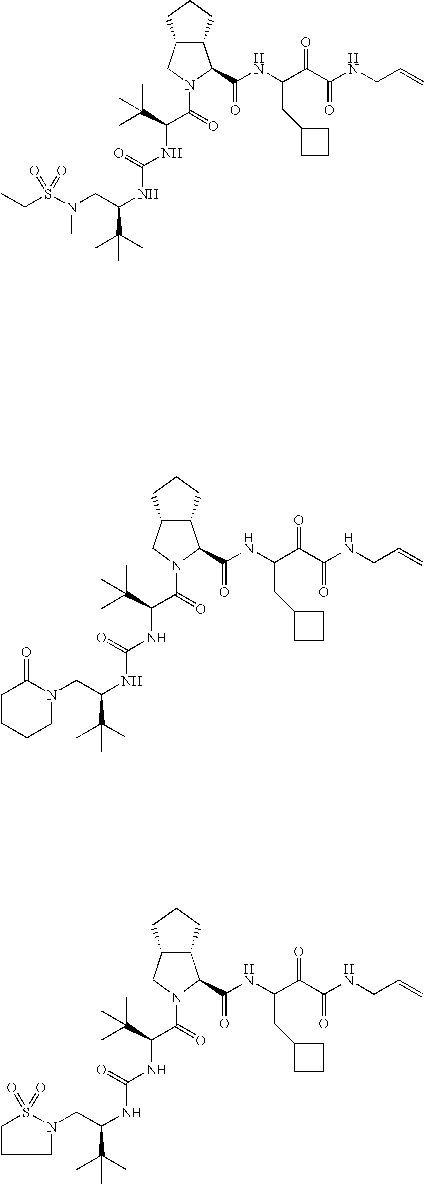 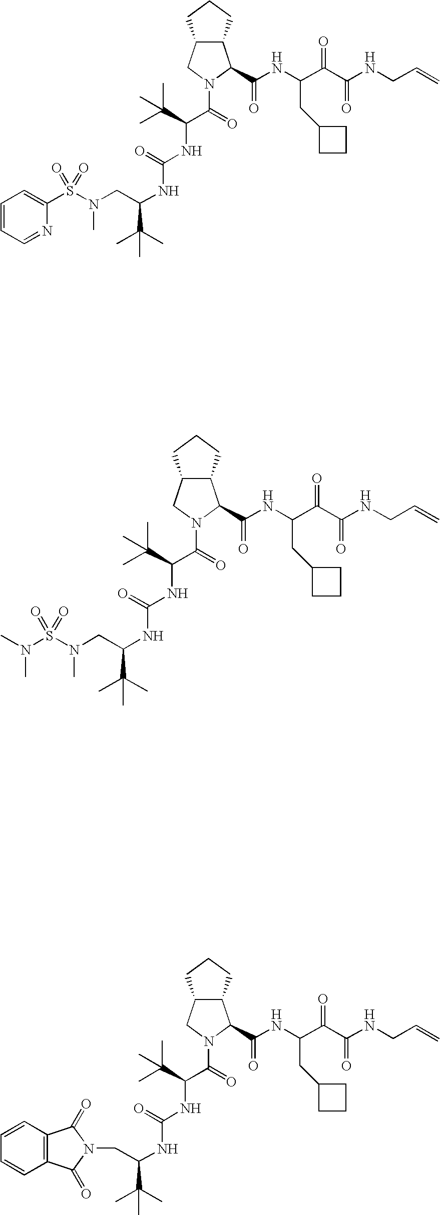 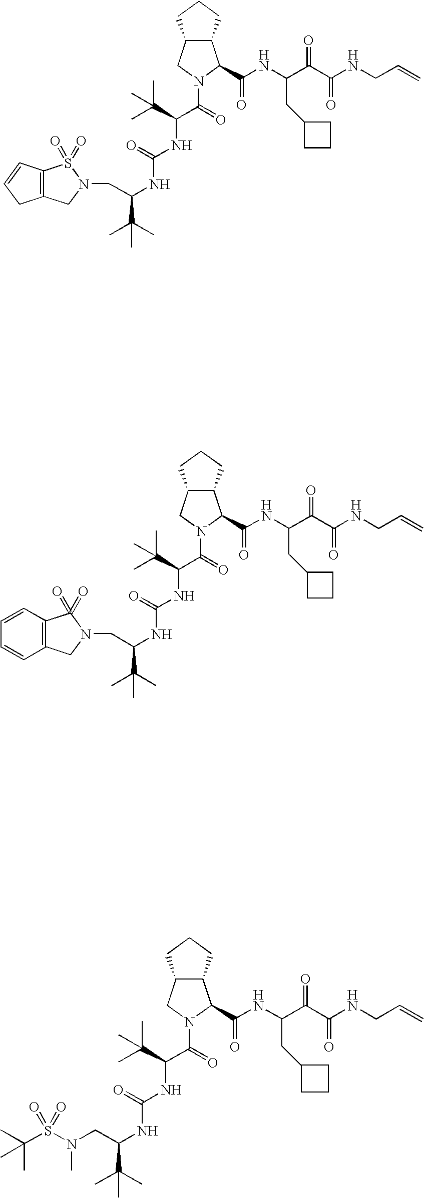 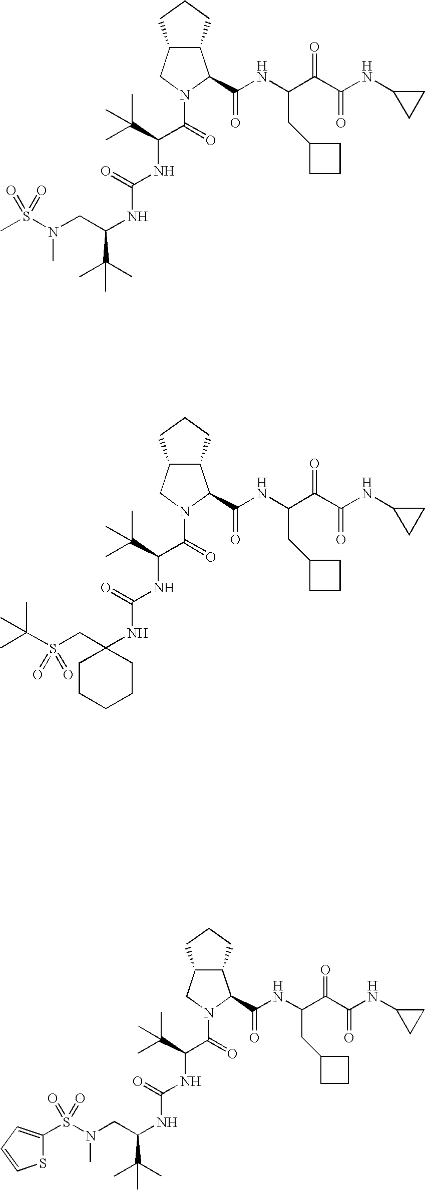 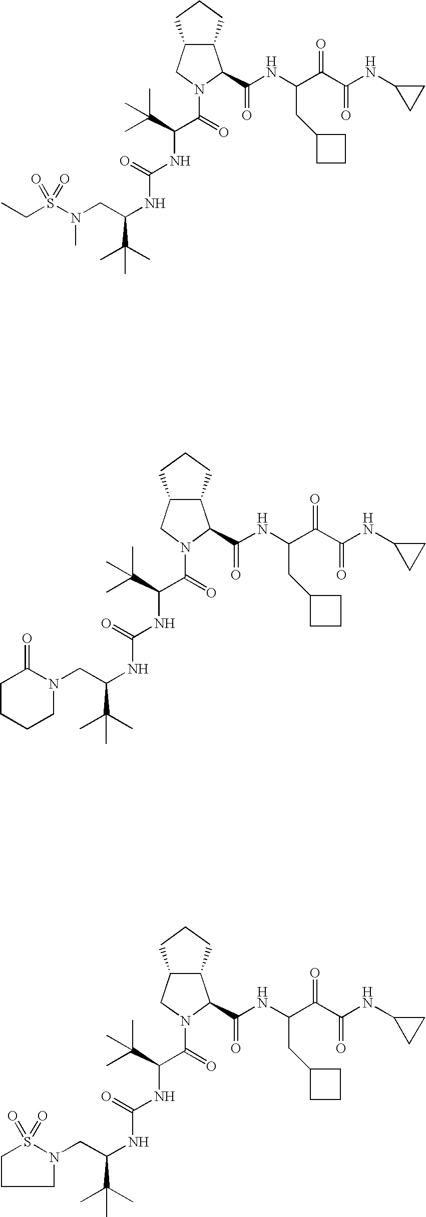 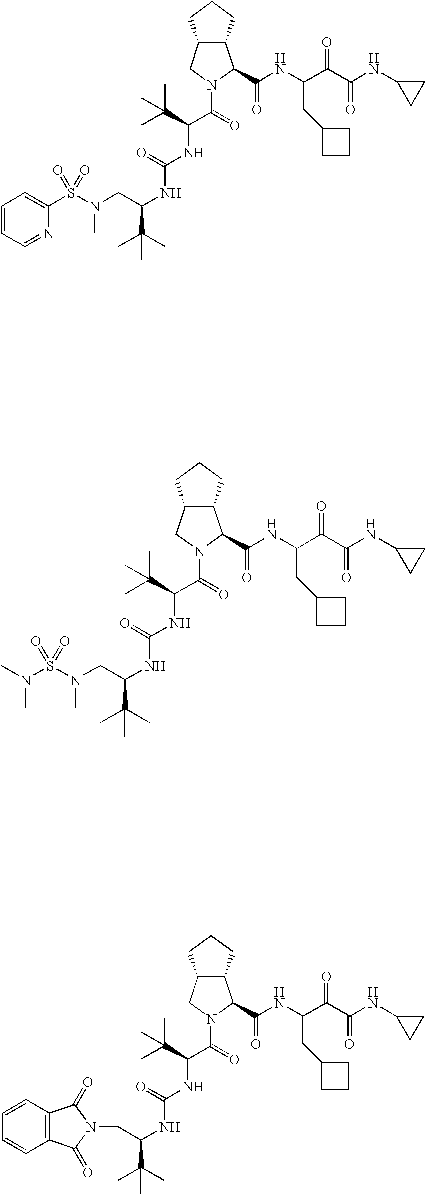 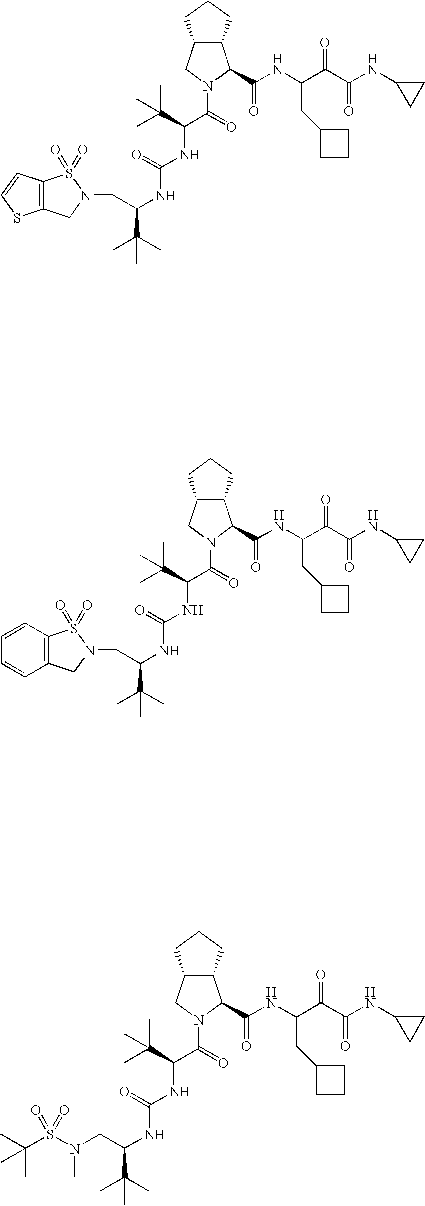 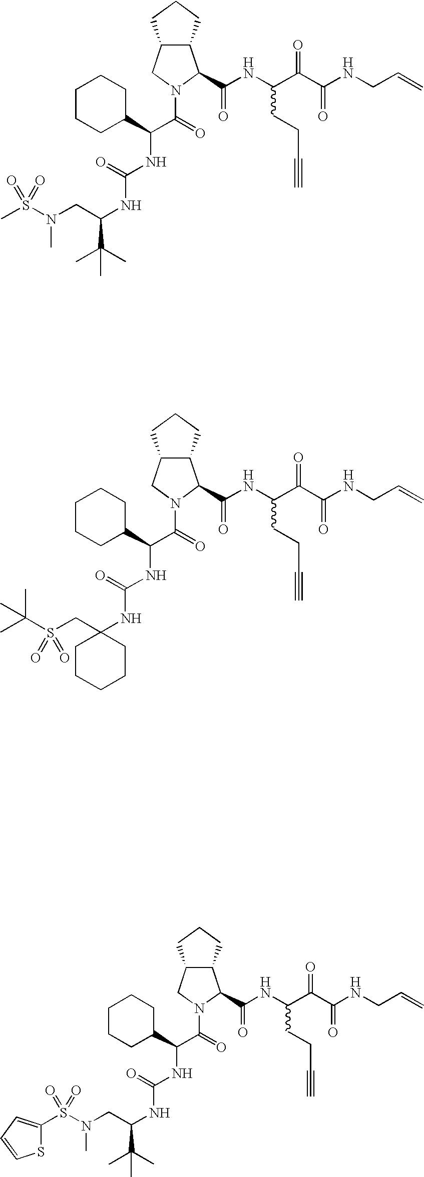 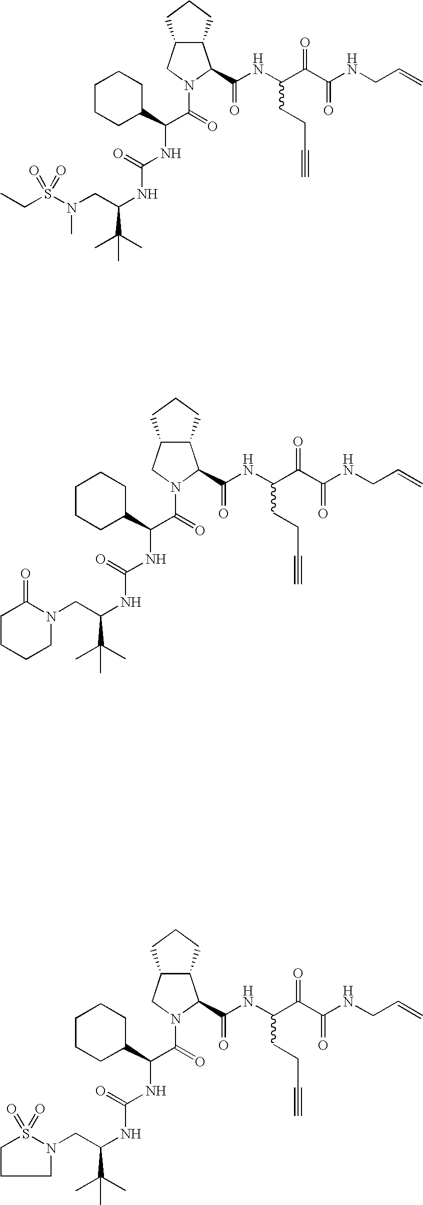 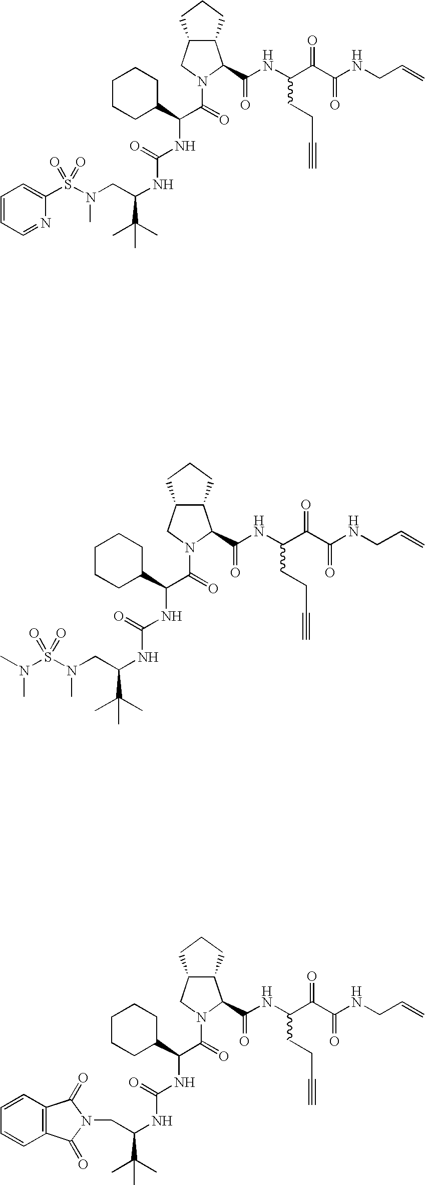 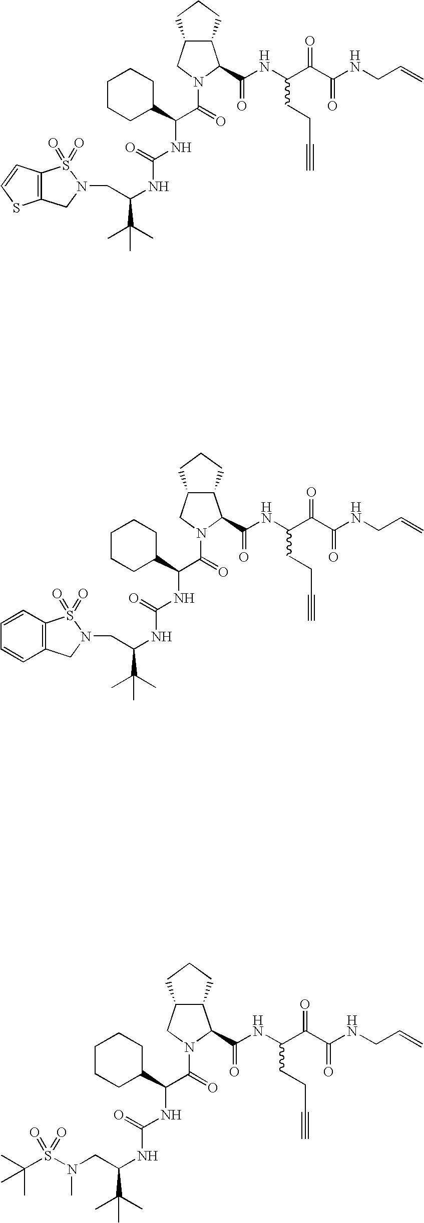 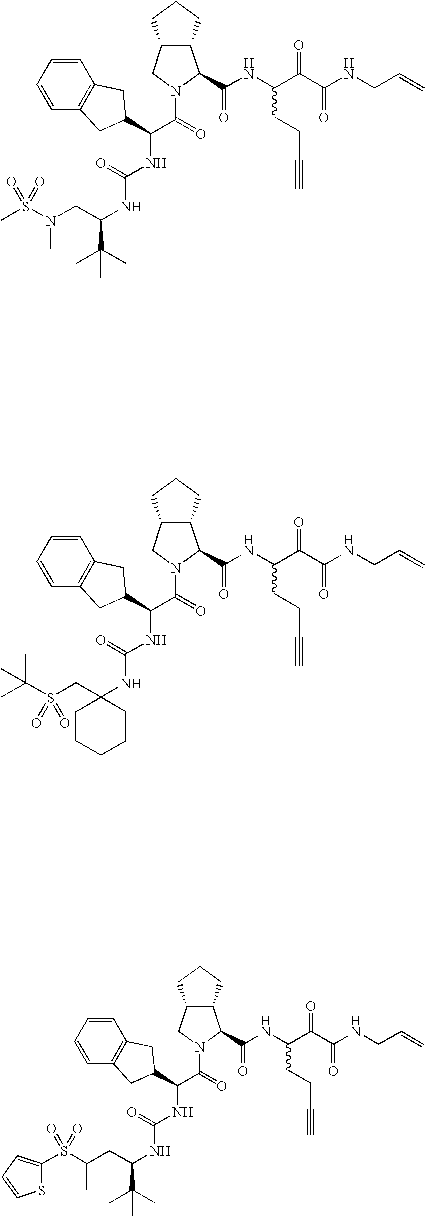 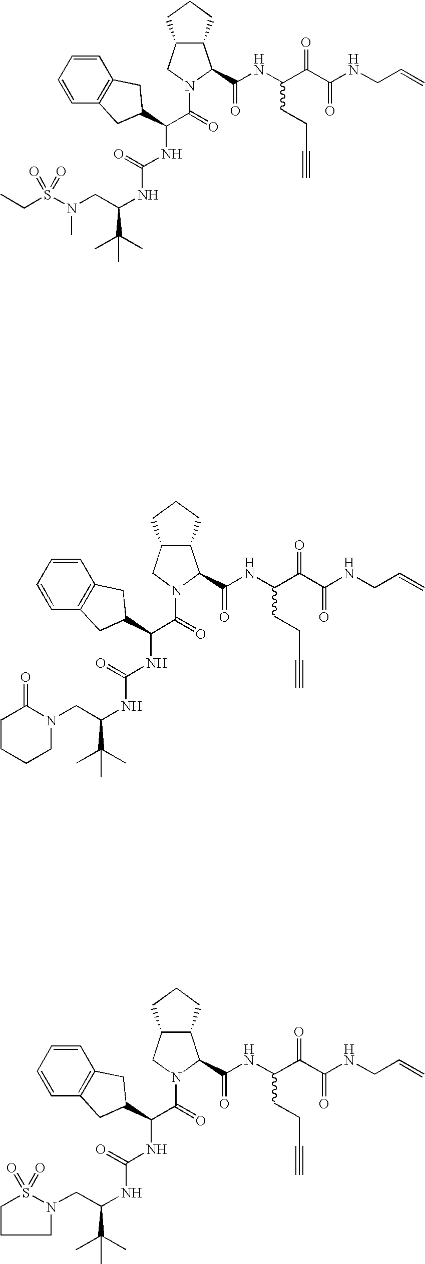 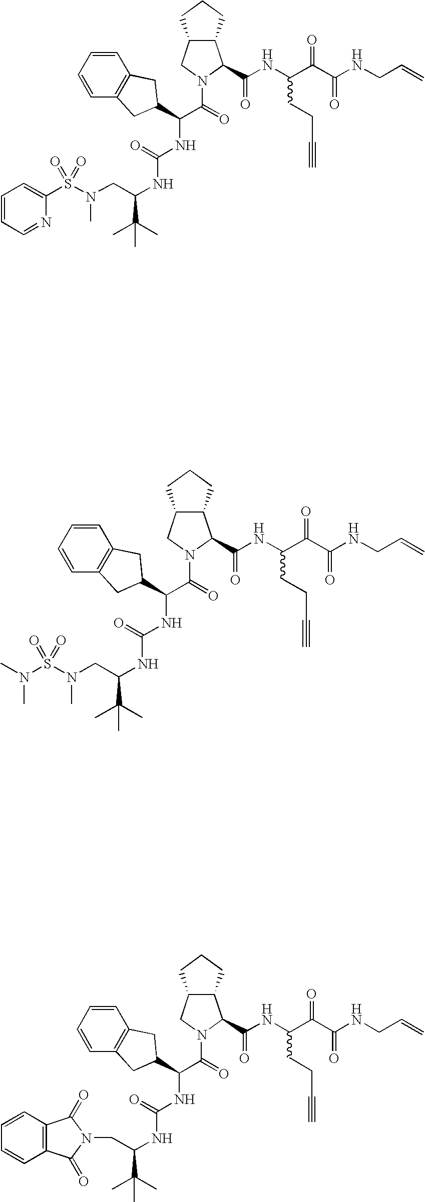 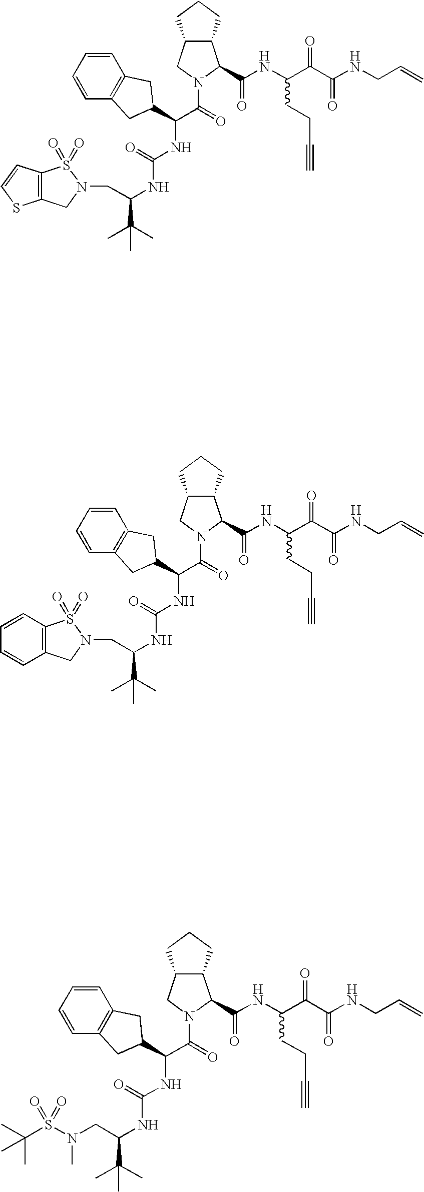 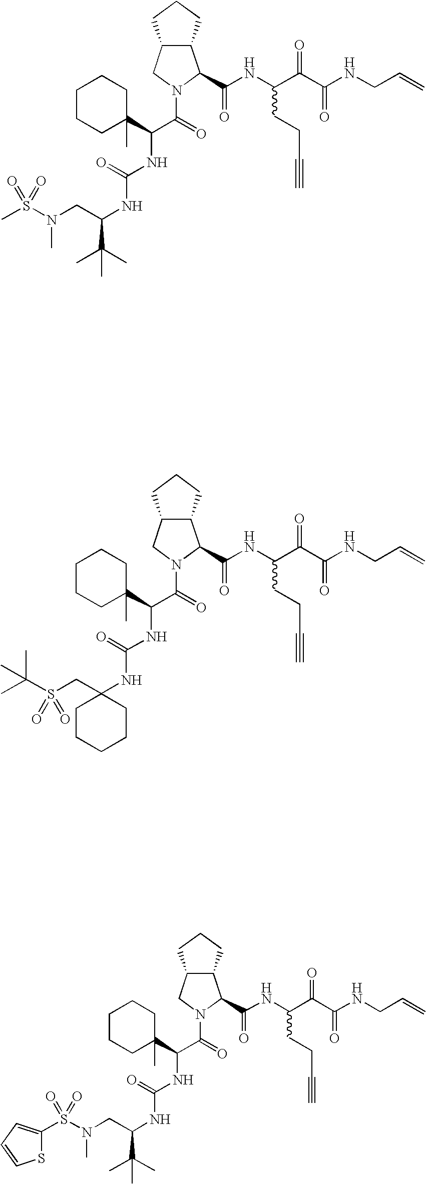 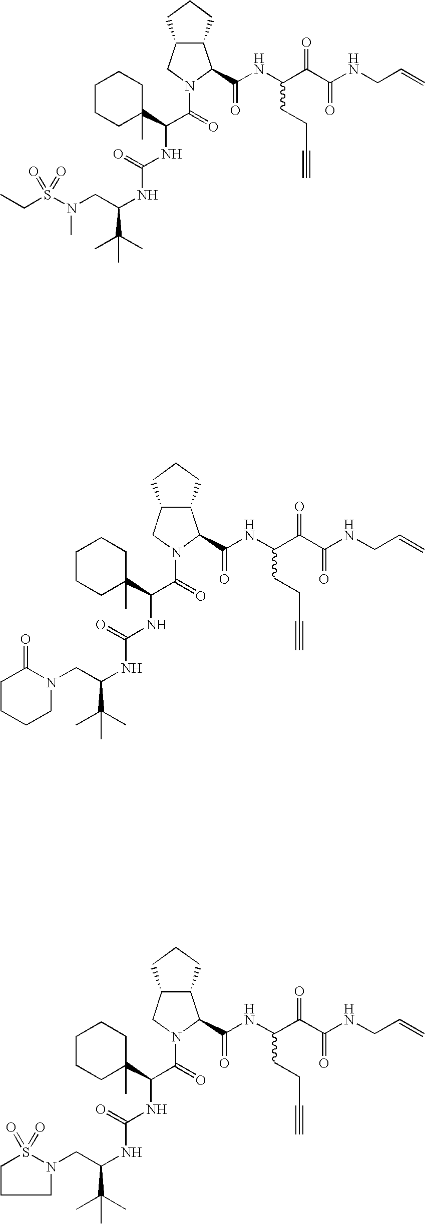 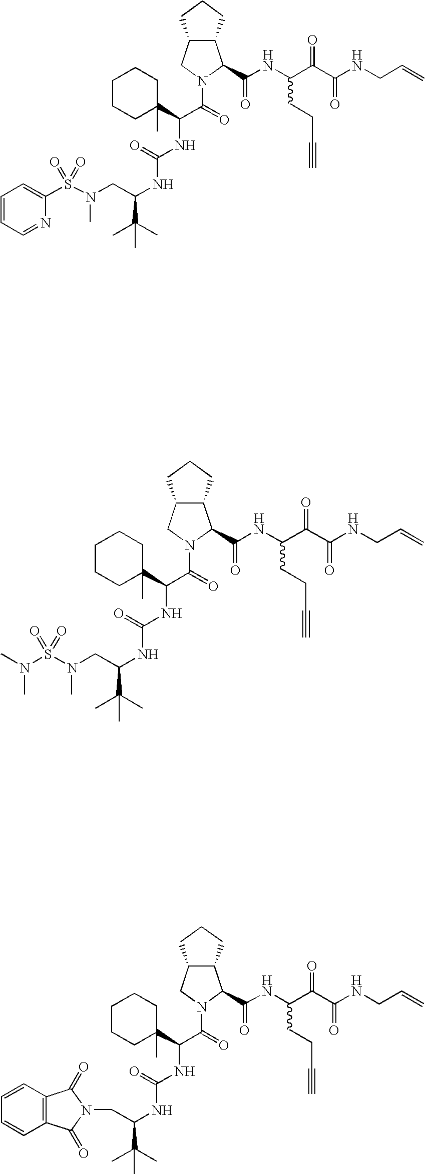 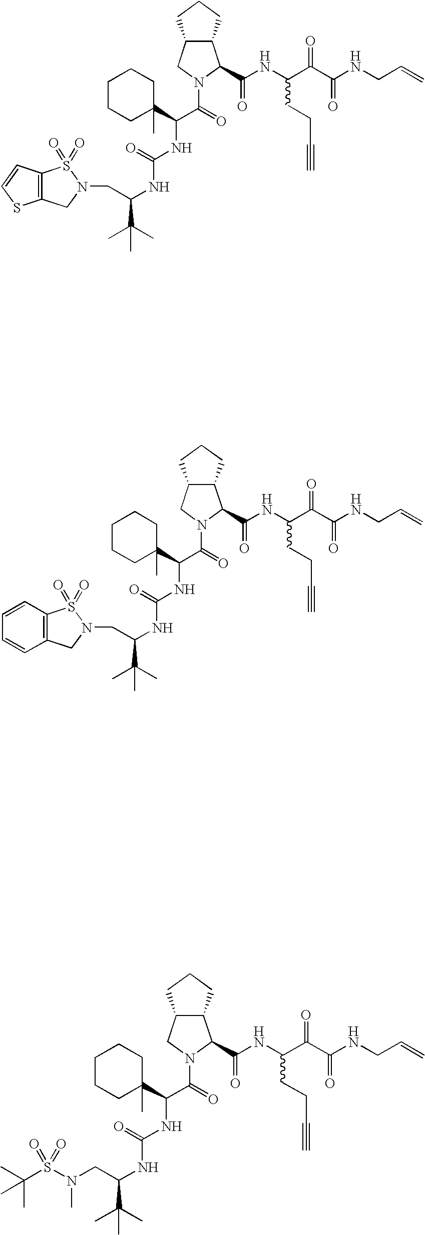
or a pharmaceutically acceptable salt, solvate or ester thereof.

Compounds of formula XVII are disclosed in U.S. patent application Ser. No. 11/064,574 filed Feb. 24, 2005. The preparation of these compounds is disclosed in the experimental section of this application set forth hereinbelow.

Non-limiting examples of certain compounds disclosed in U.S. patent application Ser. No. 11/064,574 are: 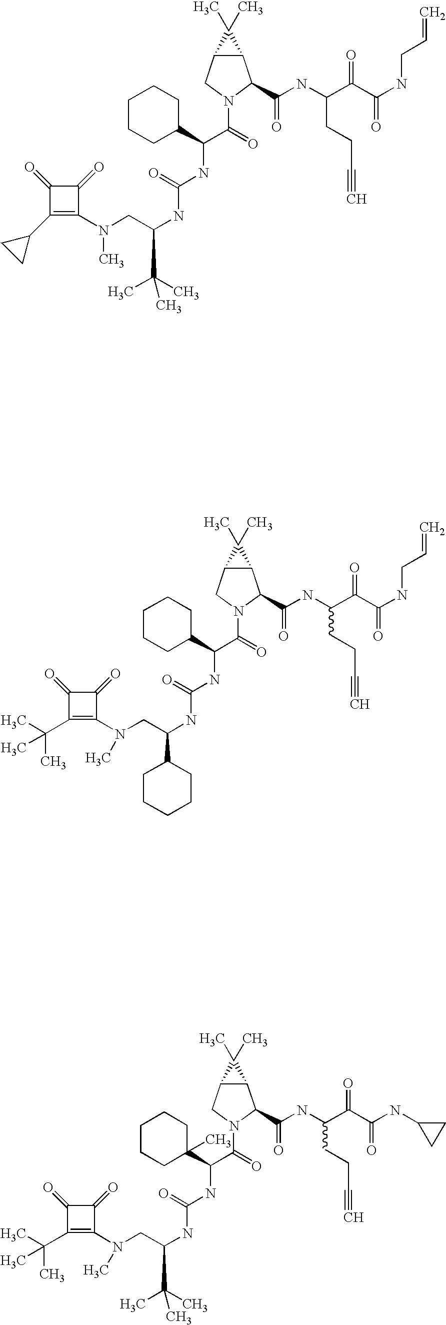 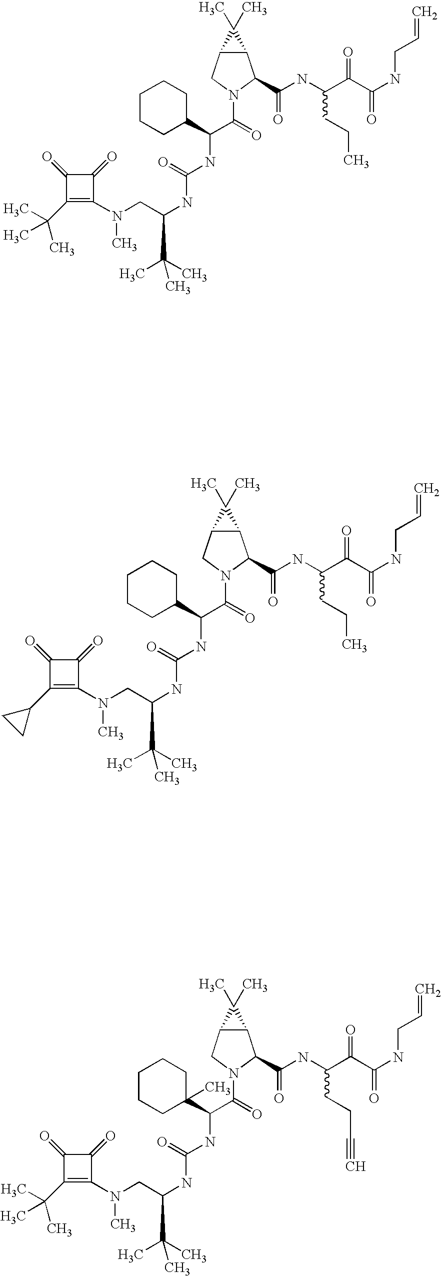 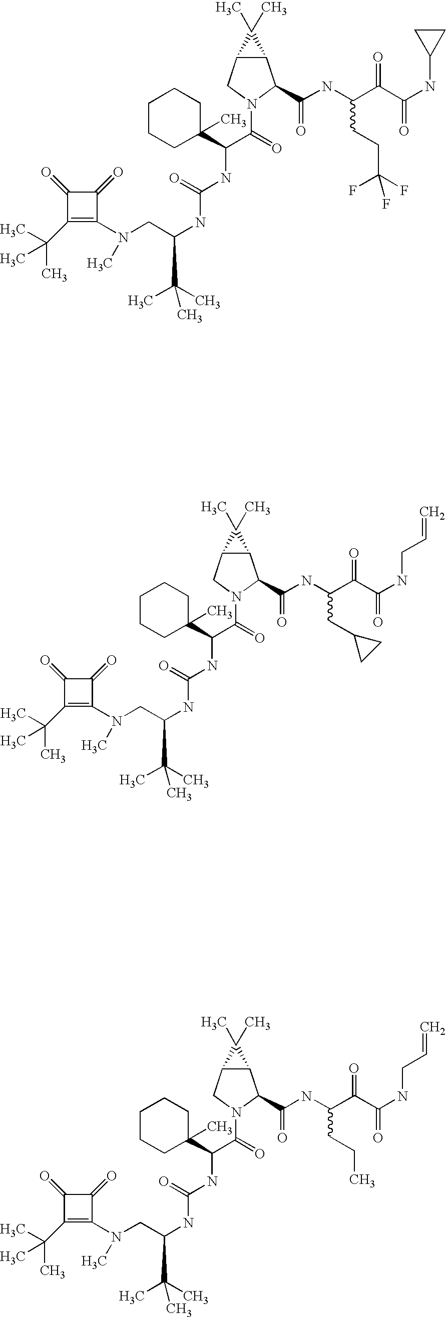 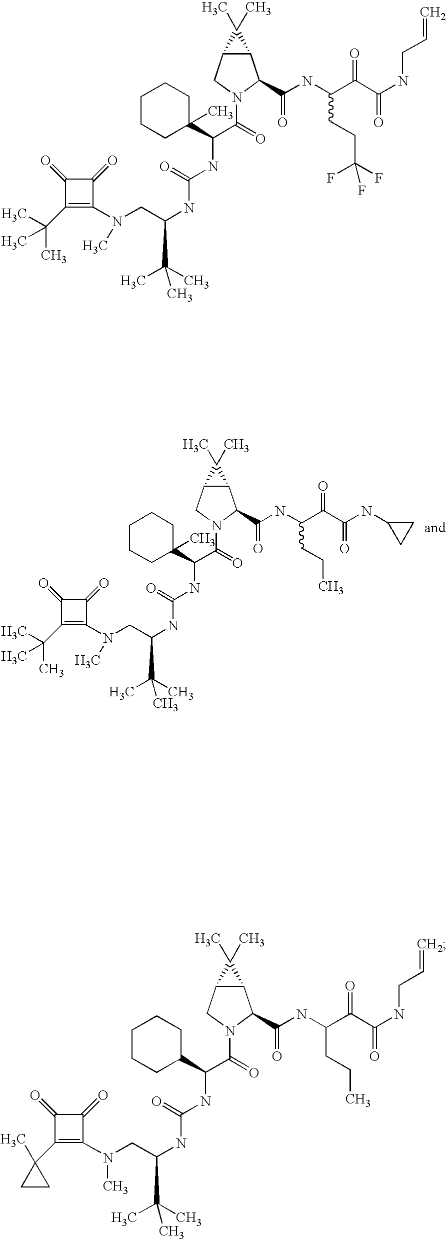
or a pharmaceutically acceptable salt, solvate or ester thereof.

Compounds of formula XVIII are disclosed in U.S. Provisional Patent Application Ser. No. 60/605,234 filed Aug. 27, 2004. The preparation of these compounds is disclosed in the experimental section of this application set forth hereinbelow.

Non-limiting examples of certain compounds disclosed in U.S. Provisional Patent Application Ser. No. 60/605,234 are: 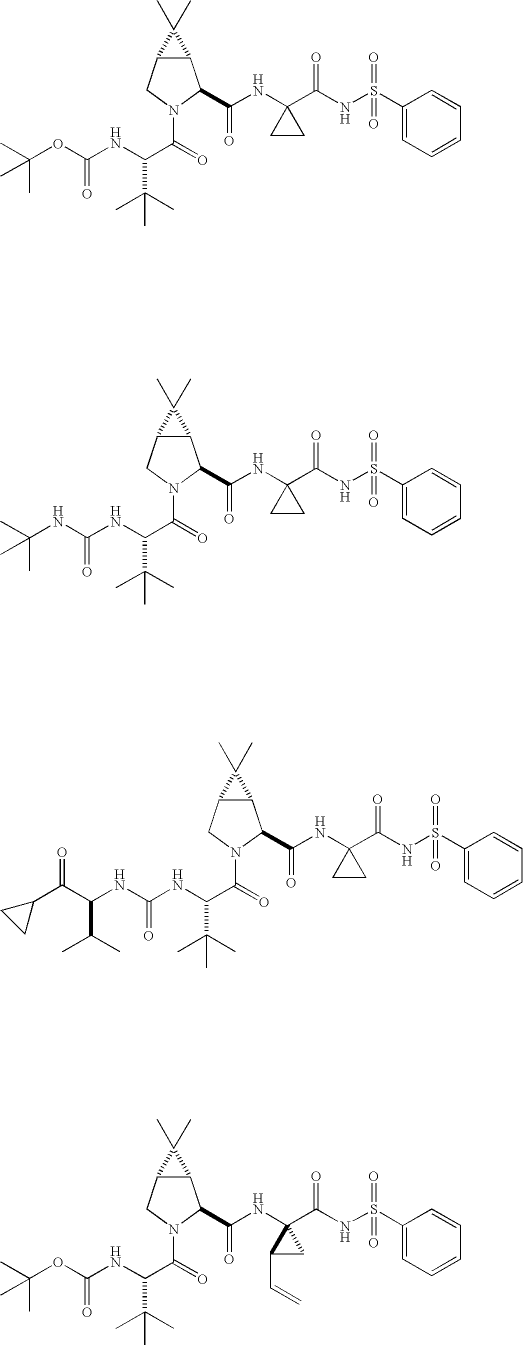 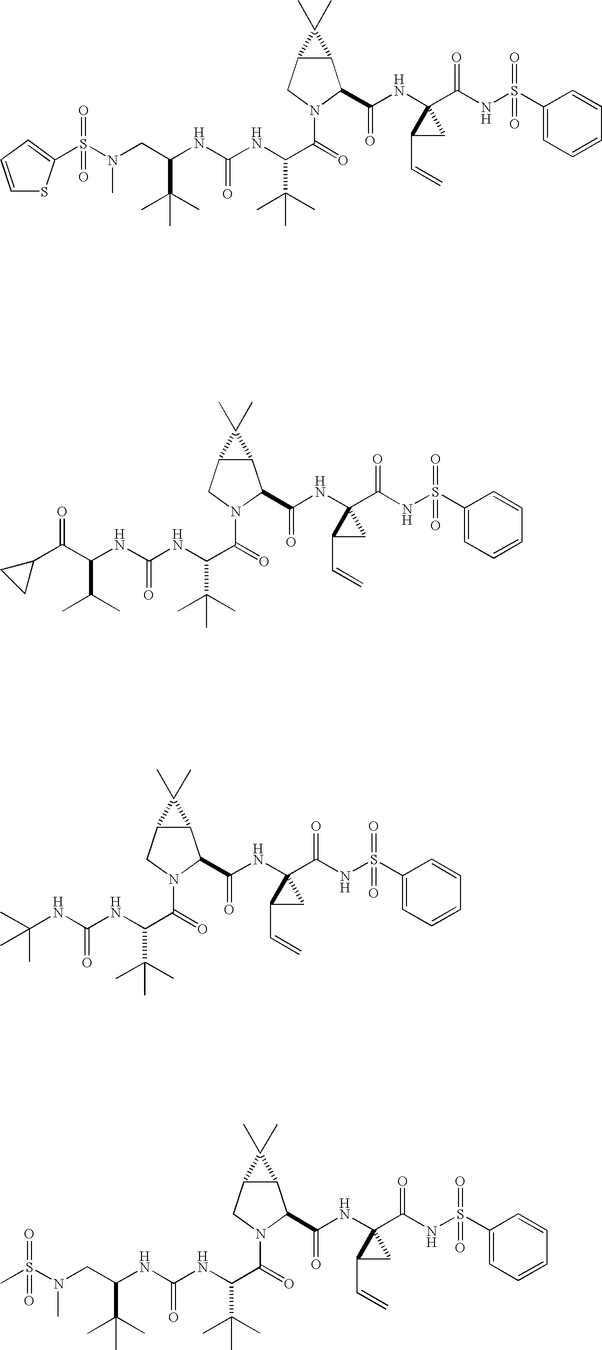 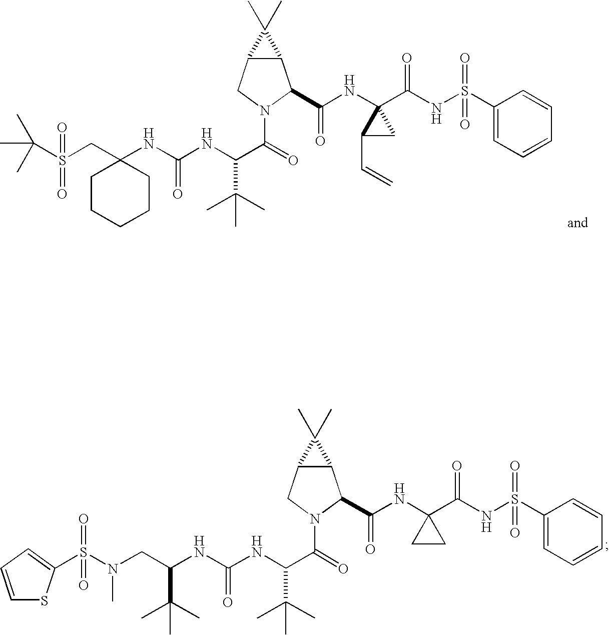
or a pharmaceutically acceptable salt, solvate or ester thereof.

Compounds of formula XIX are disclosed in U.S. Provisional Patent Application Ser. No. 60/573,191 filed May 20, 2004. The preparation of these compounds is disclosed in the experimental section of this application set forth hereinbelow.

Non-limiting examples of certain compounds disclosed in U.S. Provisional Patent Application Ser. No. 60/573,191 are: 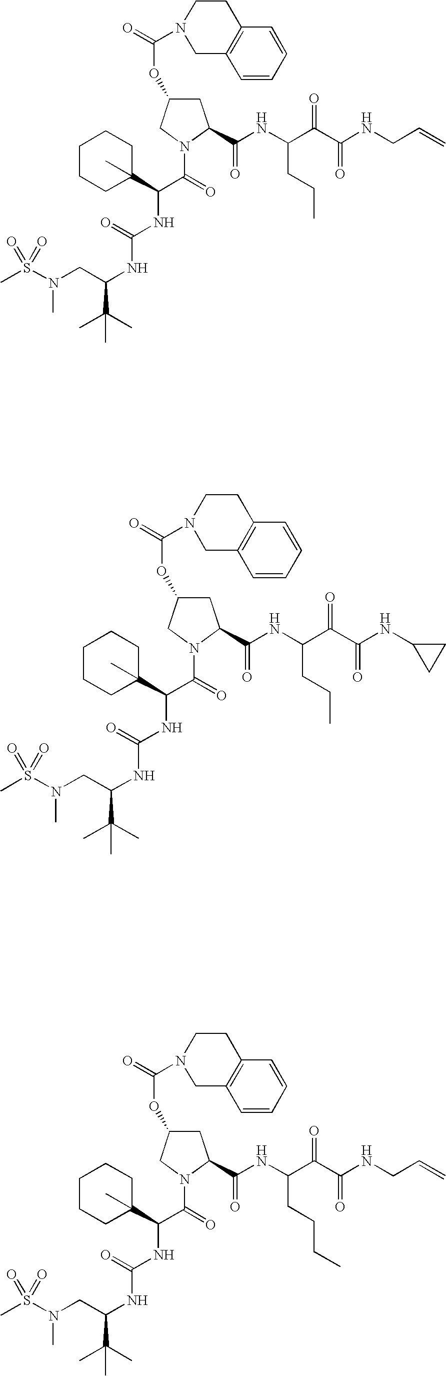 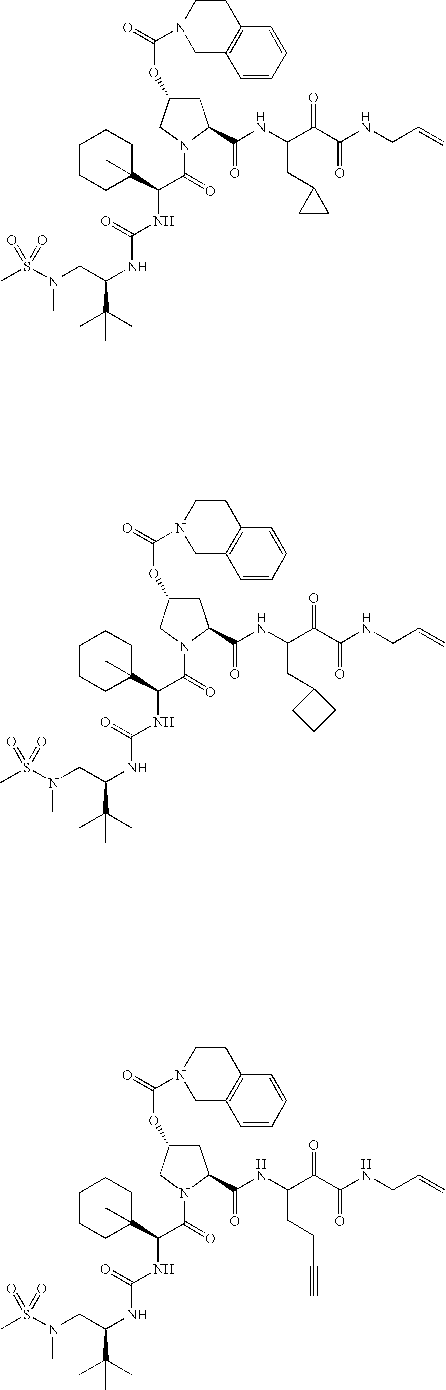 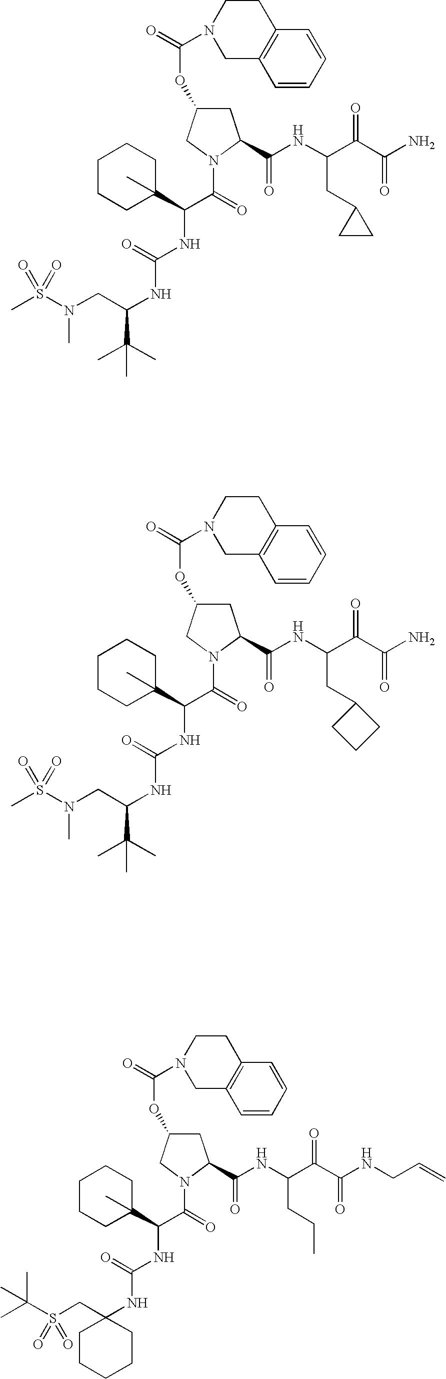 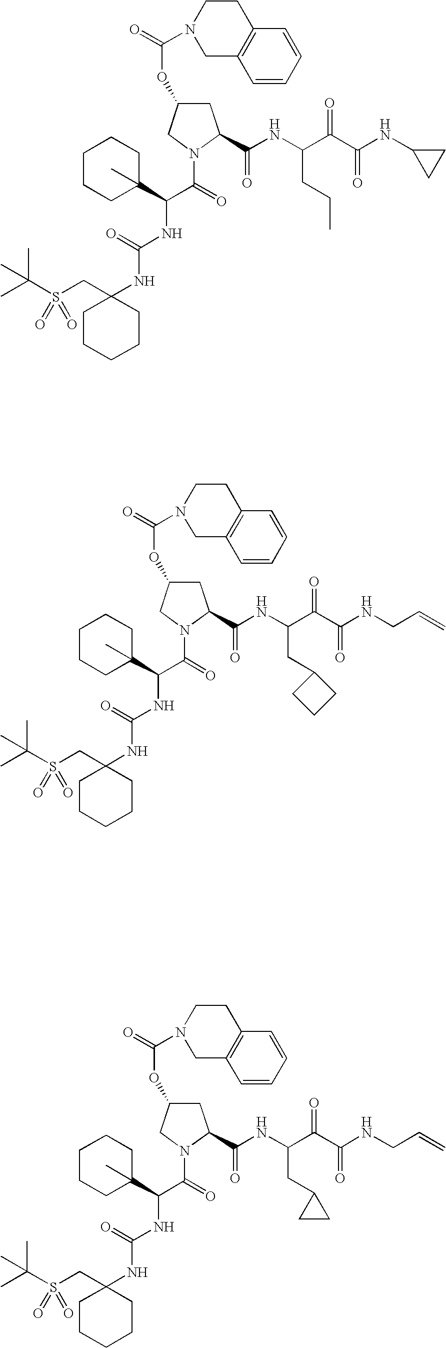 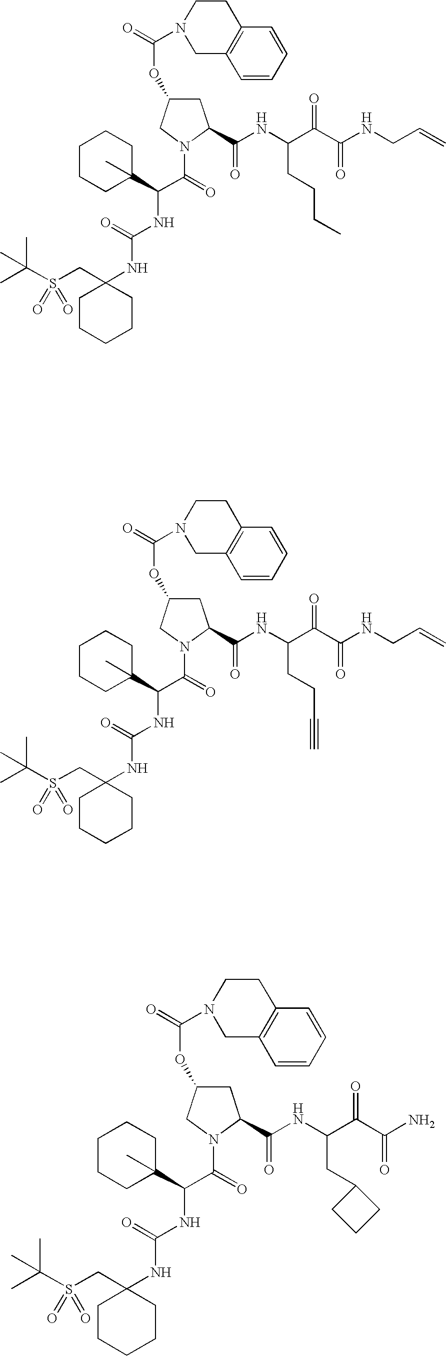 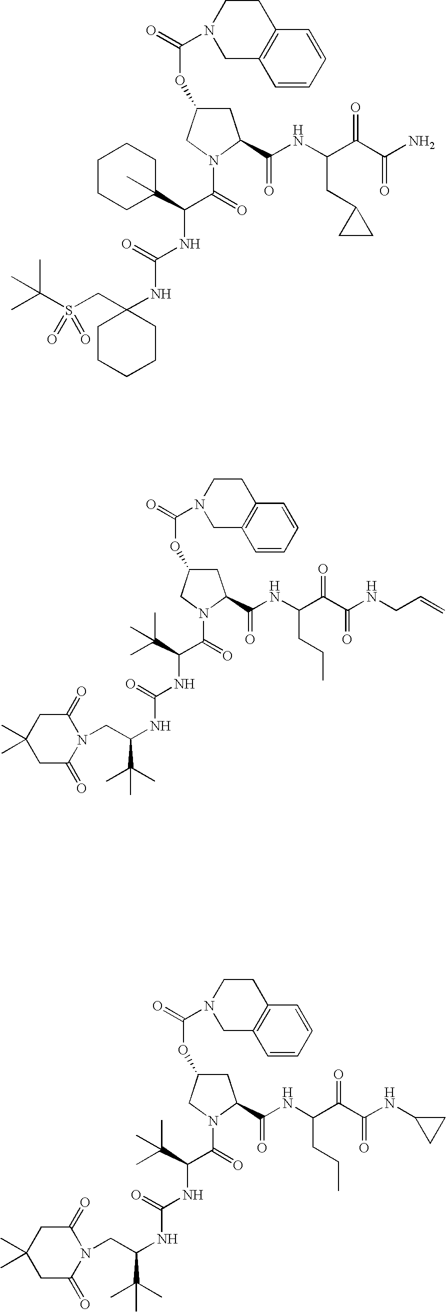 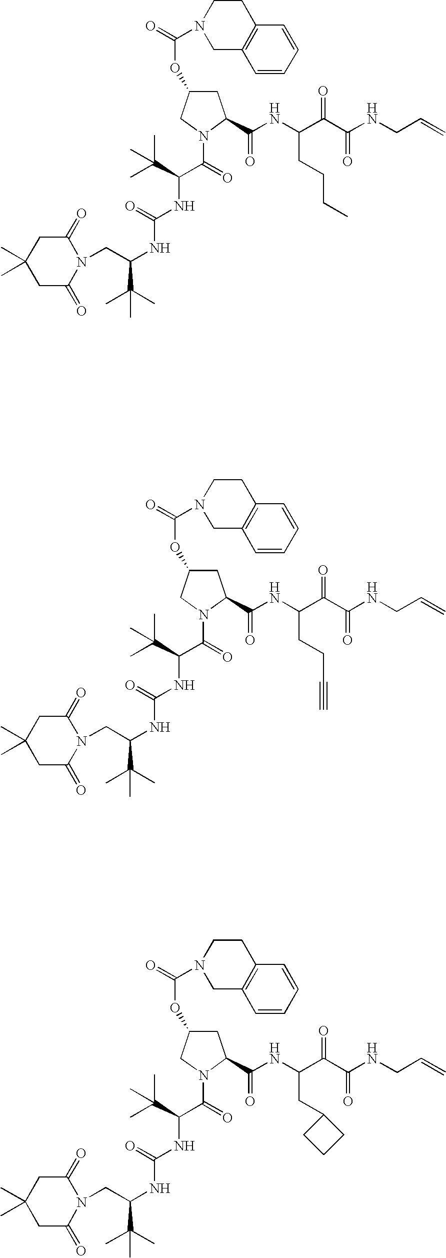 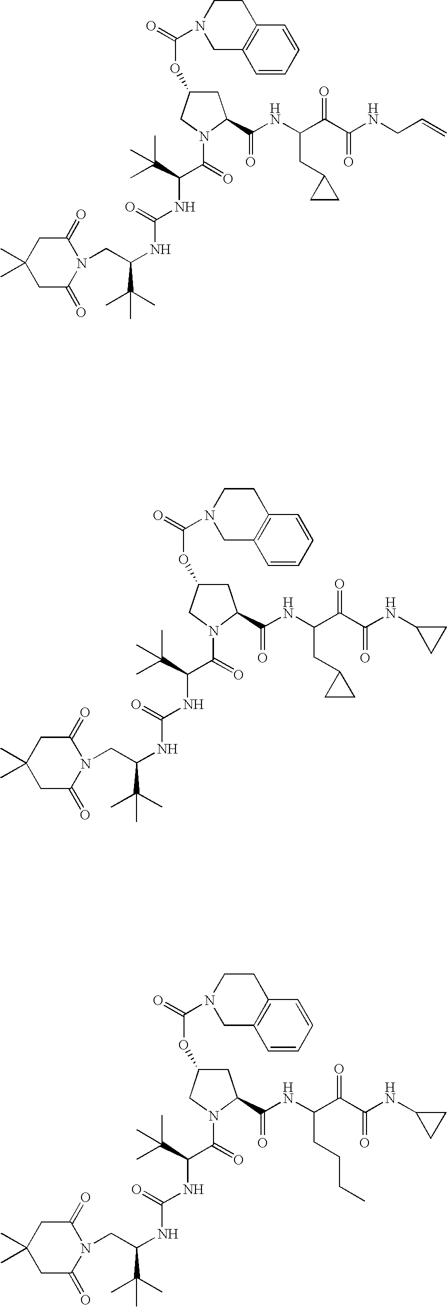 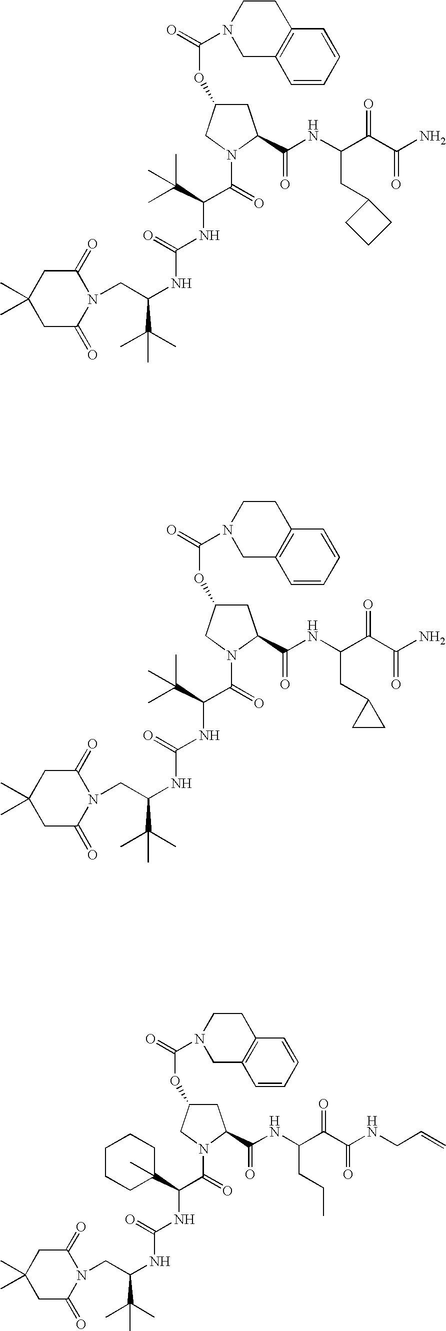 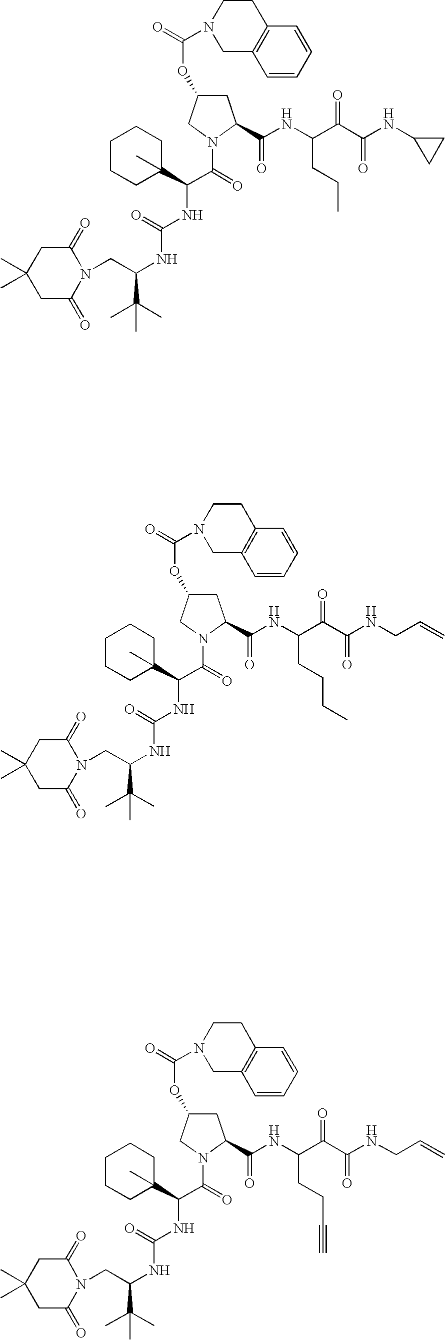 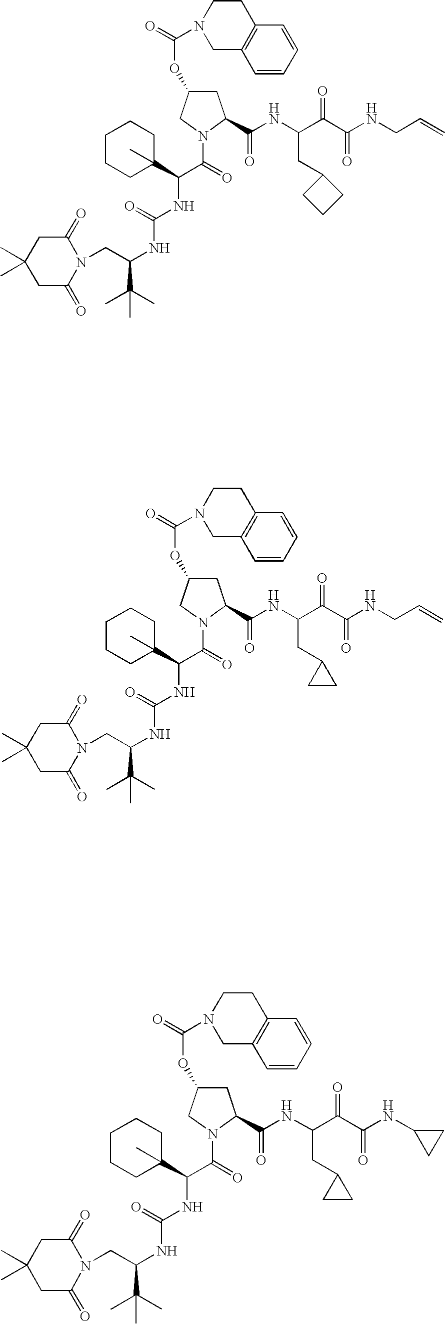 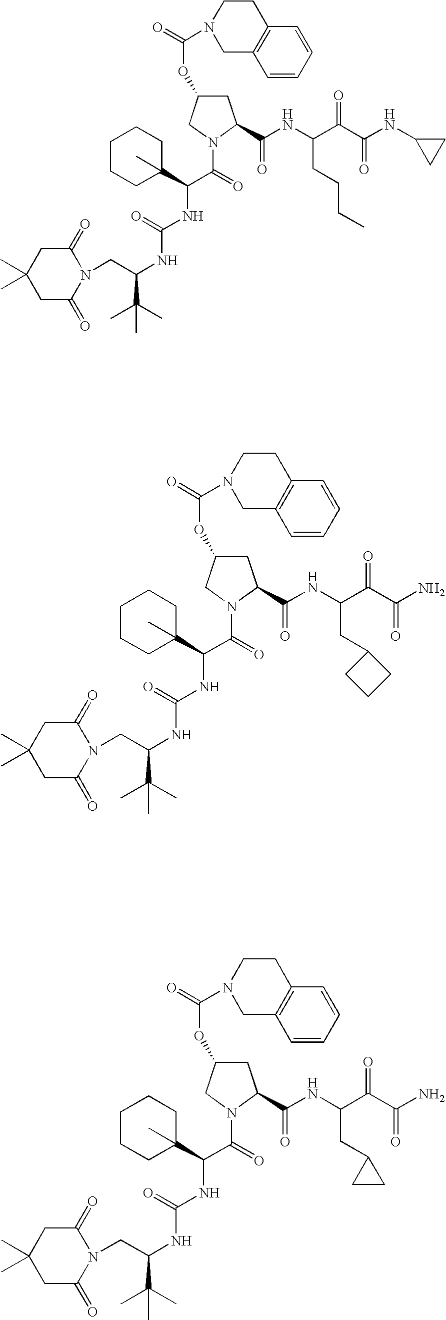 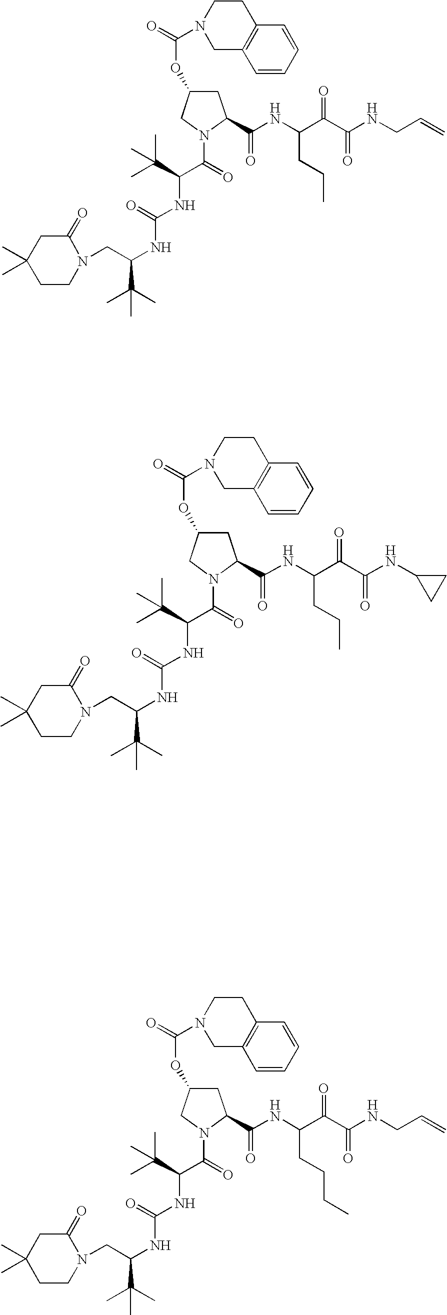 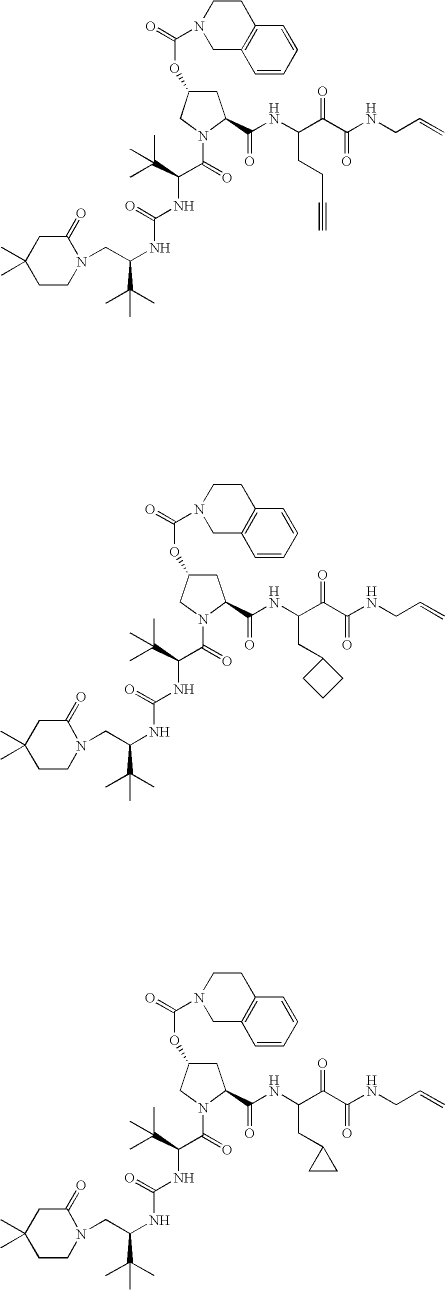 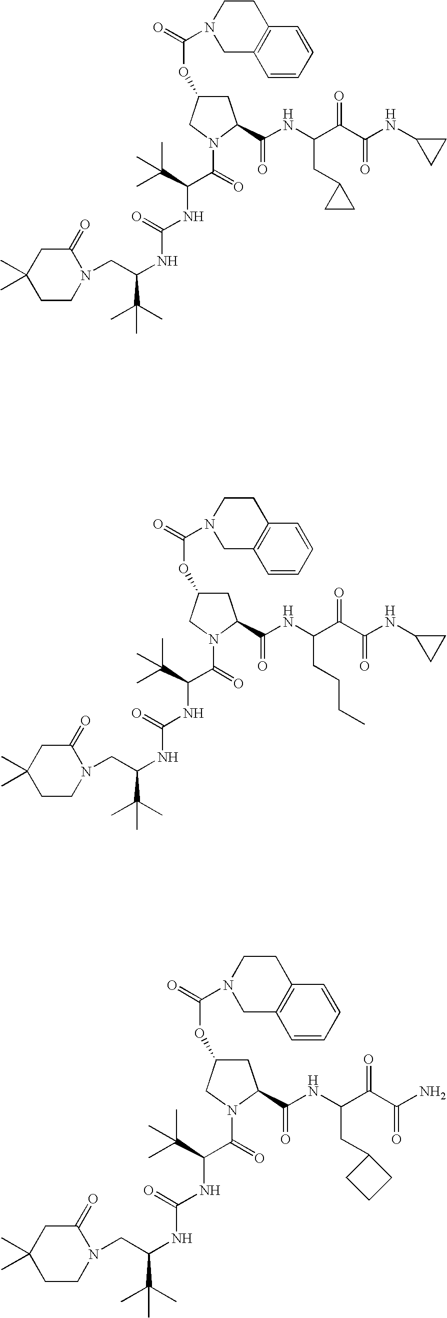 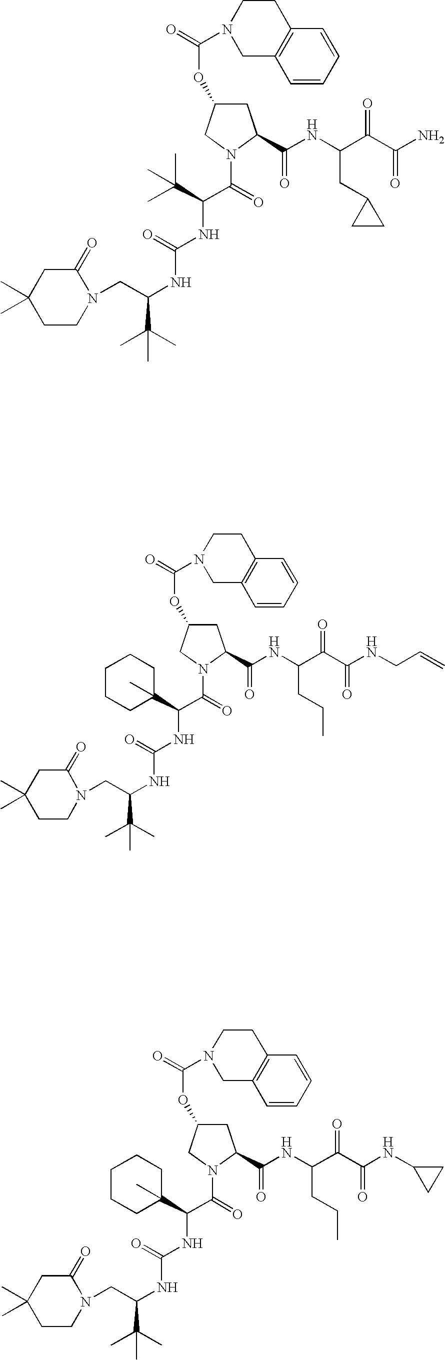 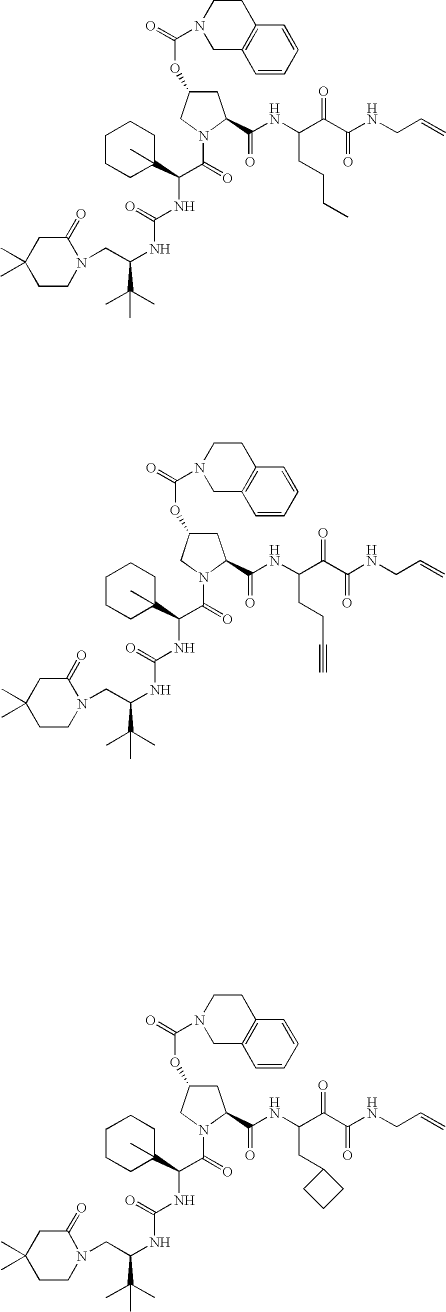 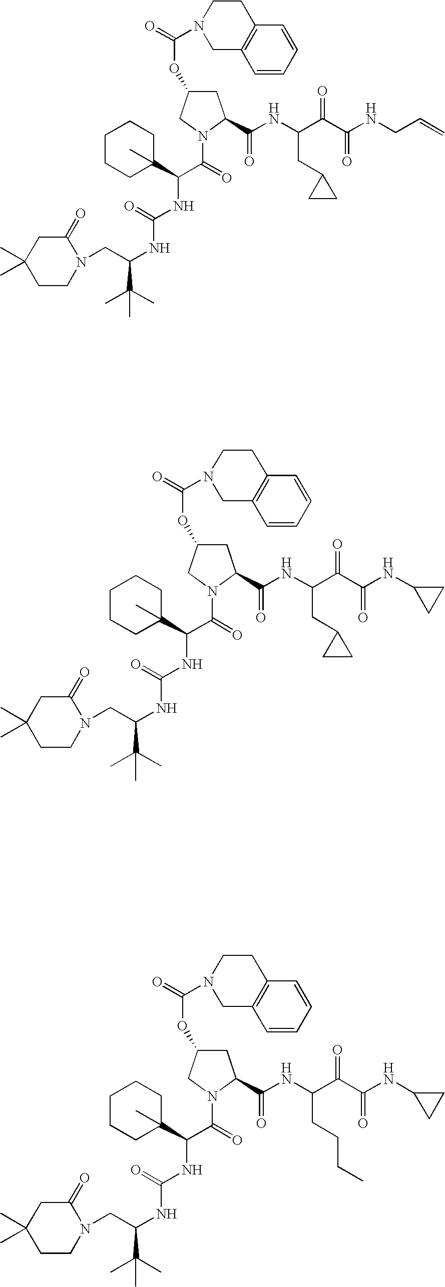 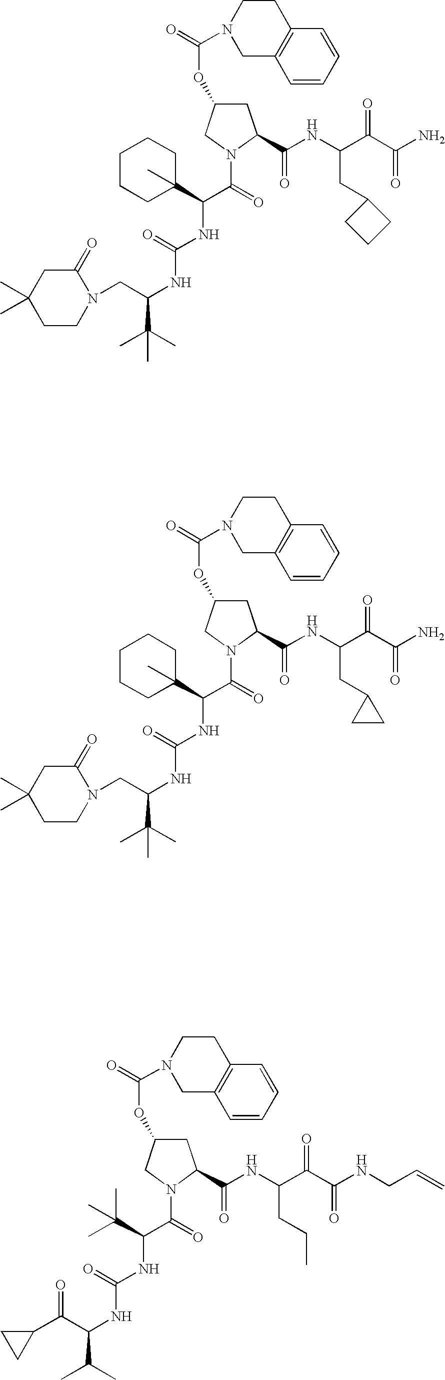 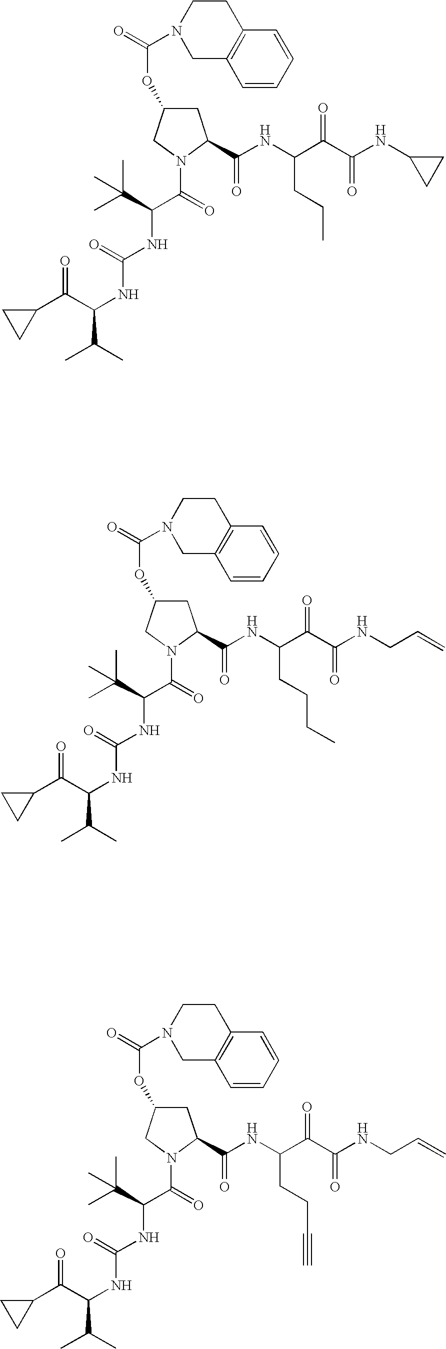 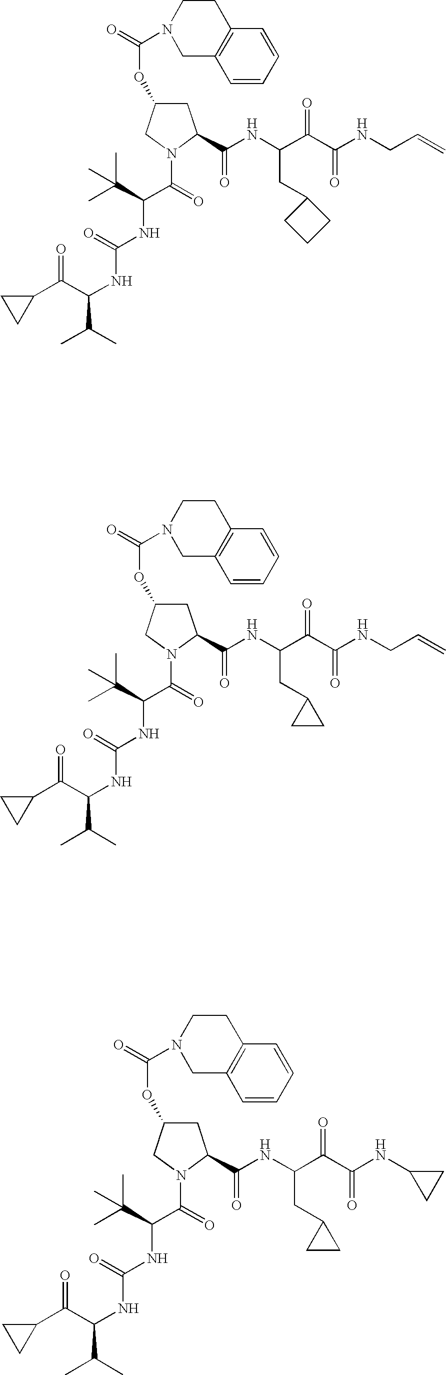 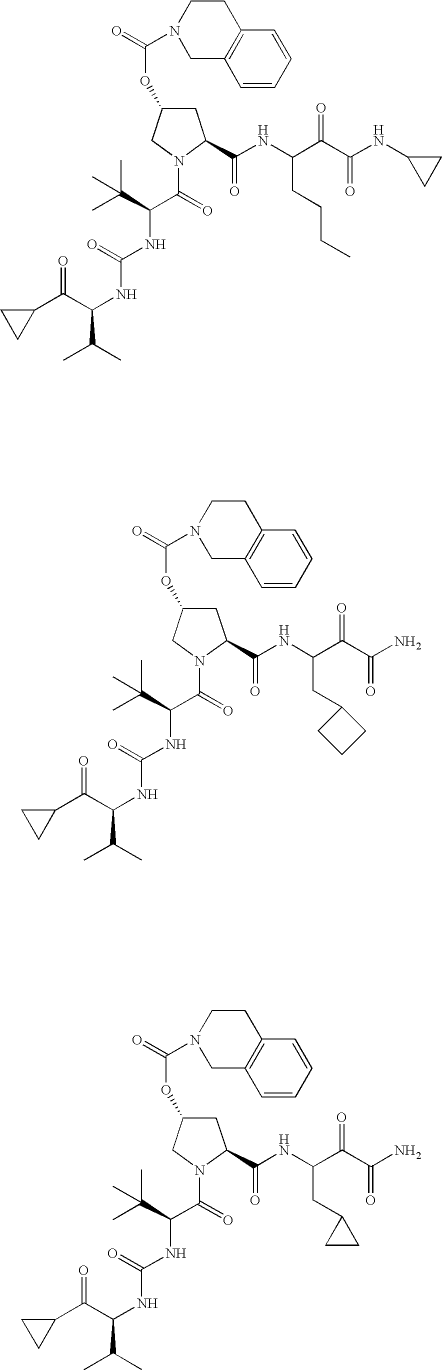 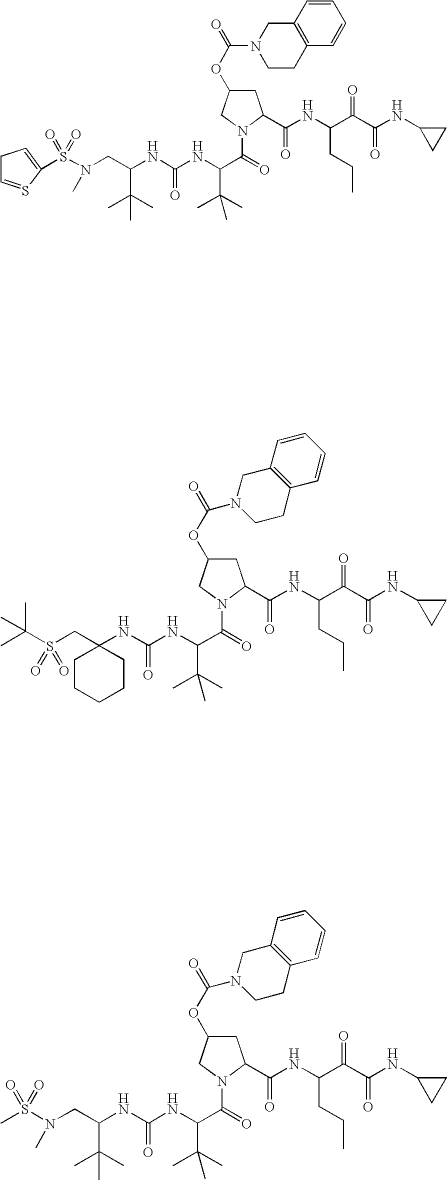 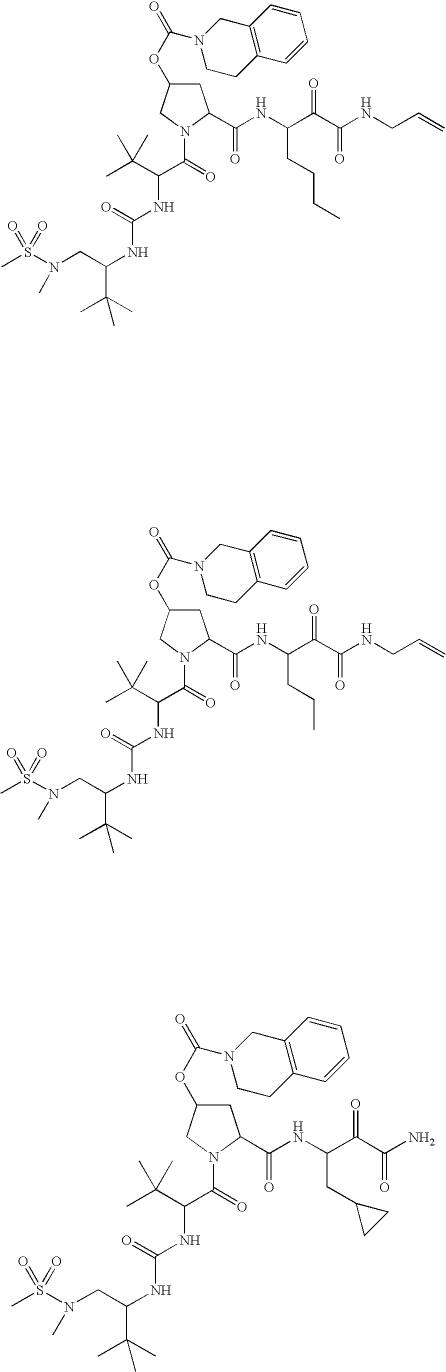 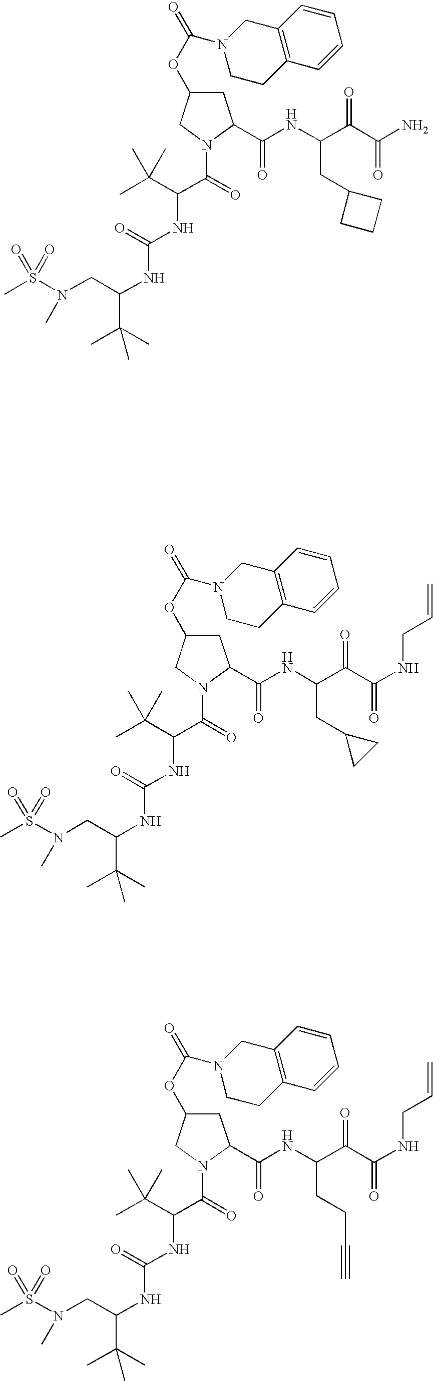 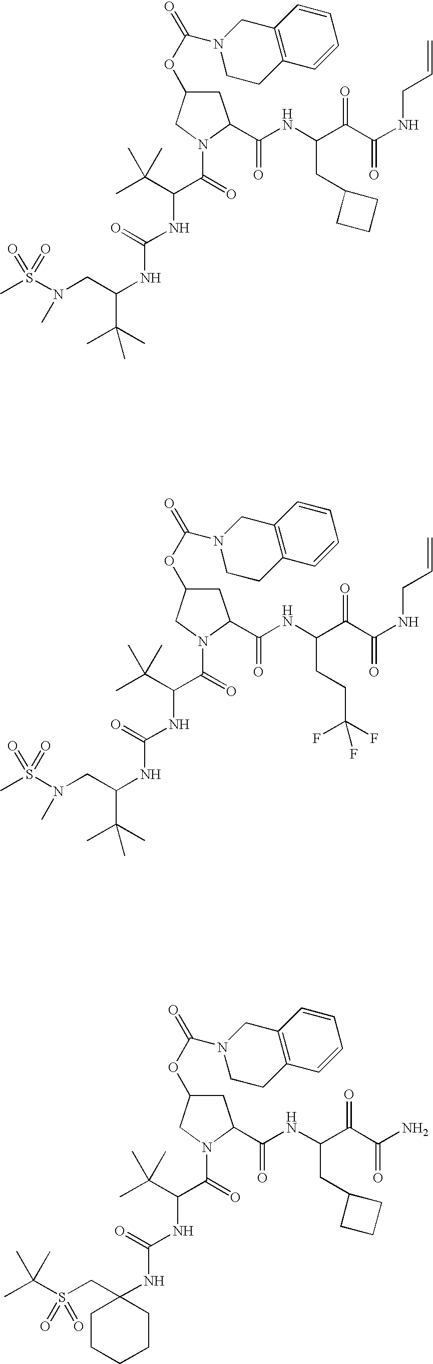 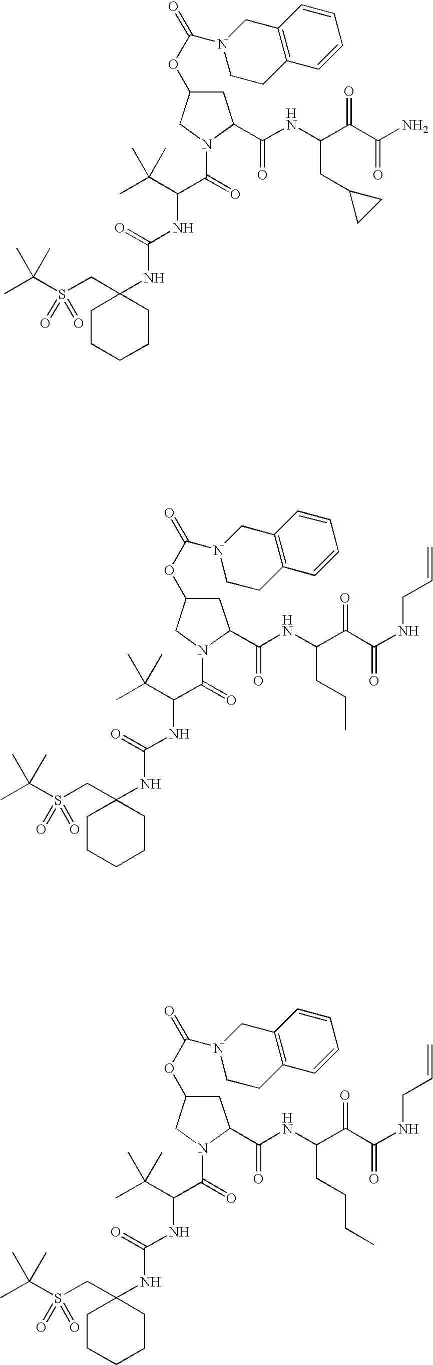 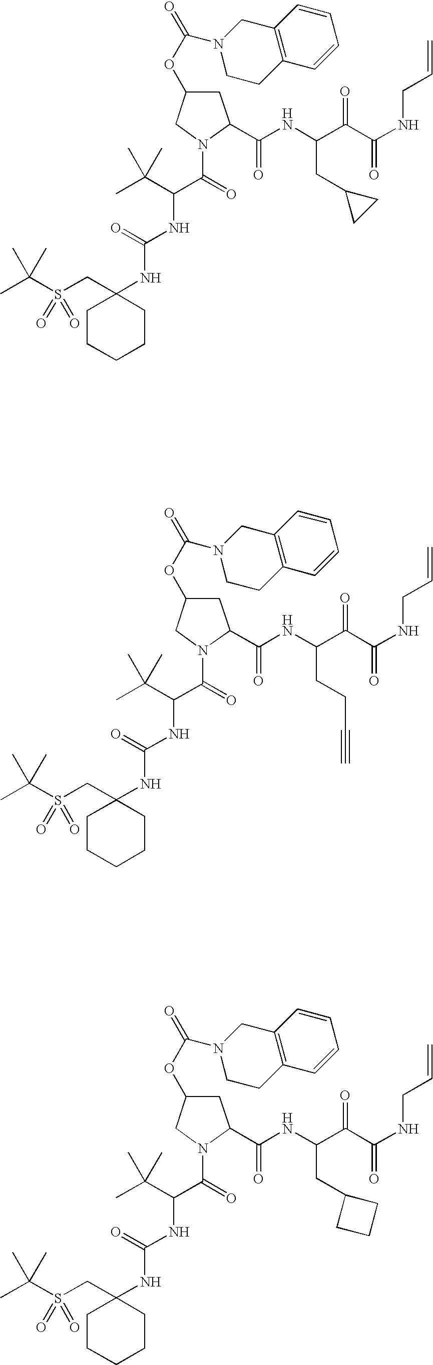 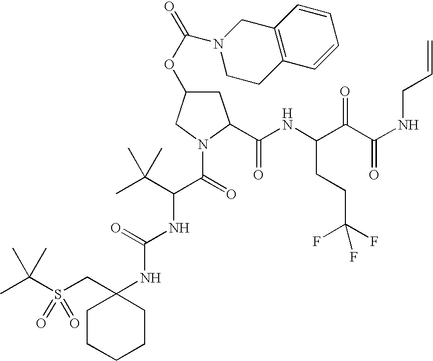
or a pharmaceutically acceptable salt, solvate or ester thereof.

Compounds of formula (XX) have been disclosed in U.S. Pat. No. 6,767,991 at col. 3, line 48 through col. 147, incorporated herein by reference.

Compounds of formula (XXI) have been disclosed in U.S. Patent Publication Nos. 2002/0016442, 2002/0037998 and U.S. Pat. Nos. 6,268,207, 6,323,180 at col. 3, line 28 through col. 141, line 60, U.S. Pat. No. 6,329,379 at col. 3, line 28 through col. 147, line 27, U.S. Pat. No. 6,329,417 at col. 3, line 25 through col. 147, line 30, U.S. Pat. No. 6,410,531 at col. 3, line 28 through col. 141, U.S. Pat. No. 6,534,523 at col. 3, line 34 through col. 139, line 29, and U.S. Pat. No. 6,420,380 at col. 3, line 28 through col. 141, line 65, each incorporated herein by reference.

Compounds of formula (XXII) have been disclosed in PCT International Patent Publication WO00/59929 published on Oct. 12, 2000, U.S. Patent Publication No. 2004/0002448 and U.S. Pat. No. 6,608,027 at col. 4 through col. 137, incorporated herein by reference.

Compounds of formula (XXIII) have been disclosed in PCT International Patent Publication WO02/18369 published on Mar. 7, 2002.

Compounds of formula (XXIV) have been disclosed U.S. Patent Publication Nos. 2002/0032175, 2004/0266731 and U.S. Pat. No. 6,265,380 at col. 3, line 35 through col. 121 and U.S. Pat. No. 6,617,309 at col. 3, line 40 through col. 121, each incorporated herein by reference.

Compounds of formula (XXV) have been disclosed U.S. Pat. No. 5,866,684 at col. 1 through col. 72 and U.S. Pat. No. 6,018,020 at col. 1 through col. 73, each incorporated herein by reference.

Compounds of formula (XXVI) have been disclosed in U.S. Pat. No. 6,143,715 at col. 3, line 6 through col. 62, line 20, incorporated herein by reference.

Isomers of the various compounds of the present invention (where they exist), including enantiomers, stereoisomers, rotamers, tautomers and racemates are also contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the present invention. Isomers may also include geometric isomers, e.g., when a double bond is present. Polymorphous forms of the compounds of the present invention, whether crystalline or amorphous, also are contemplated as being part of this invention. The (+) isomers of the present compounds are preferred compounds of the present invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other. For example, both isomers (1) and (2) are contemplated: 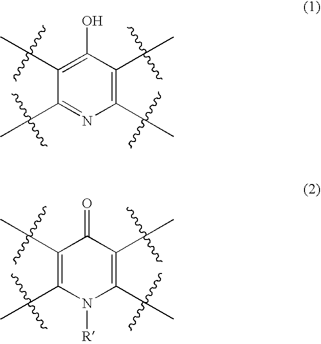
wherein R′ is H or C₁₋₆ unsubstituted alkyl.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C1-C2)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N-(C₁-C₆)alkylaminoalkyl, —C(Y⁴)Y⁵ wherein Y⁴ is H or methyl and Y⁵ is mono-N— or di-N,N-(C₁-C₆)alkylamino morpholino, piperidin-1-yl or pyrrolidin-1-yl, and the like.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving a compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate). “Effective amount” or “therapeutically effective amount” is meant to describe an amount of a compound or a composition of the present invention effective in inhibiting HCV protease and/or cathepsins, and thus producing the desired therapeutic, ameliorative, inhibitory or preventative effect in a suitable subject.

The compounds of the present invention can form salts that are also within the scope of this invention. Reference to a compound of the present invention herein is understood to include reference to salts, esters and solvates thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the various formulae of the present invention may be formed, for example, by reacting a compound of the present invention with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Wash., D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl) ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention. All acid and base salts, as well as esters and solvates, are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

In such esters, unless otherwise specified, any alkyl moiety present preferably contains from 1 to 18 carbon atoms, particularly from 1 to 6 carbon atoms, more particularly from 1 to 4 carbon atoms. Any cycloalkyl moiety present in such esters preferably contains from 3 to 6 carbon atoms. Any aryl moiety present in such esters preferably comprises a phenyl group.

The present invention provides pharmaceutical formulations comprising the inventive peptides as an active ingredient. Because of their HCV inhibitory activity, such pharmaceutical compositions possess utility in treating hepatitis C and related disorders.

Another embodiment of the invention discloses the use of the pharmaceutical compositions disclosed above for treatment of diseases such as, for example, hepatitis C and the like. The method comprises administering a therapeutically effective amount of the inventive pharmaceutical formulation to a patient having such a disease or diseases and in need of such a treatment.

The pharmaceutical formulations of the present invention are suited for treatment of infection by any of the genotypes of HCV. HCV types and subtypes may differ in their antigenicity, level of viremia, severity of disease produced, and response to interferon therapy. (Holland, J. et al., “Hepatitis C genotyping by direct sequencing of the product from the Roche Amplicor Test: methodology and application to a South Australian population,” Pathology, 30:192-195, 1998). The nomenclature of Simmonds, P. et al. (“Classification of hepatitis C virus into six major genotypes and a series of subtypes by phylogenetic analysis of the NS-5 region,” J. Gen. Virol., 74:2391-9, 1993) is widely used and classifies isolates into six major genotypes, 1 through 6, with two or more related subtypes, e.g., 1a, 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3. (Lamballerie, X. et al., “Classification of hepatitis C variants in six major types based on analysis of the envelope 1 and nonstructural 5B genome regions and complete polyprotein sequences,” J. Gen. Virol., 78:45-51, 1997). The major genotypes have been defined as having sequence similarities of between 55 and 72% (mean 64.5%), and subtypes within types as having 75%-86% similarity (mean 80%) when sequenced in the NS-5 region. (Simmonds, P. et al., “Identification of genotypes of hepatitis C by sequence comparisons in the core, E1 and NS-5 regions,” J. Gen. Virol., 75:1053-61, 1994).

In an alternative embodiment, the formulations of the present invention can be useful for inhibiting cathepsin activity, for example for treating cancer and other cathepsin-associated disorders as discussed below.

In yet another embodiment, the compounds of the invention may be used for the treatment of HCV in humans in monotherapy mode or in a combination therapy (e.g., dual combination, triple combination etc.) mode such as, for example, in combination with antiviral and/or immunomodulatory agents. Examples of such antiviral and/or immunomodulatory agents include Ribavirin (from Schering-Plough Corporation, Madison, N.J.) and Levovirin™ (from ICN Pharmaceuticals, Costa Mesa, Calif.), VP 50406™ (from Viropharma, Incorporated, Exton, Pa.), ISIS 14803™ (from ISIS Pharmaceuticals, Carlsbad, Calif.), Heptazyme™ (from Ribozyme Pharmaceuticals, Boulder, Colo.), VX 497™ (from Vertex Pharmaceuticals, Cambridge, Mass.), Thymosin™ (from SciClone Pharmaceuticals, San Mateo, Calif.), Maxamine (Maxim Pharmaceuticals, San Diego, Calif.), mycophenolate mofetil (from Hoffman-LaRoche, Nutley, N.J.), interferon (such as, for example, interferon-alpha, PEG-interferon alpha conjugates) and the like. “PEG-interferon alpha conjugates” are interferon alpha molecules covalently attached to a PEG molecule. Illustrative PEG-interferon alpha conjugates include interferon alpha-2a (Roferon™, from Hoffman La-Roche, Nutley, N.J.) in the form of pegylated interferon alpha-2a (e.g., as sold under the trade name PegaSyS™), interferon alpha-2b (Intron™, from Schering-Plough Corporation) in the form of pegylated interferon alpha-2b (e.g., as sold under the trade name PEG-Intron™), interferon alpha-2c (Berofor Alpha™, from Boehringer Ingelheim, Ingelheim, Germany) or consensus interferon as defined by determination of a consensus sequence of naturally occurring interferon alphas (Infergen™, from Amgen, Thousand Oaks, Calif.).

The HCV protease inhibitor can be administered in combination with interferon alpha, PEG-interferon alpha conjugates or consensus interferon concurrently or consecutively at recommended dosages for the duration of HCV treatment in accordance with the methods of the present invention. The commercially available forms of interferon alpha include interferon alpha 2a and interferon alpha 2b and also pegylated forms of both aforementioned interferon alphas. The recommended dosage of INTRON-A interferon alpha 2b (commercially available from Schering-Plough Corp.) as administered by subcutaneous injection at 3MIU(12 mcg)/0.5 mL/TIW is for 24 weeks or 48 weeks for first time treatment. The recommended dosage of PEG-INTRON interferon alpha 2b pegylated (commercially available from Schering-Plough Corp.) as administered by subcutaneous injection at 1.5 mcg/kg/week, within a range of 40 to 150 mcg/week, is for at least 24 weeks. The recommended dosage of ROFERON A inteferon alpha 2a (commercially available from Hoffmann-La Roche) as administered by subcutaneous or intramuscular injection at 3MIU(11.1 mcg/mL)/TIW is for at least 48 to 52 weeks, or alternatively 6MIU/TIW for 12 weeks followed by 3MIU/TIW for 36 weeks. The recommended dosage of PEGASUS interferon alpha 2a pegylated (commercially available from Hoffmann-La Roche) as administered by subcutaneous injection at 180mcg/1 mL or 180 mcg/0.5 mL is once a week for at least 24 weeks. The recommended dosage of INFERGEN interferon alphacon-1 (commercially available from Amgen) as administered by subcutaneous injection at 9 mcg/TIW is for 24 weeks for first time treatment and up to 15 mcg/TIW for 24 weeks for non-responsive or relapse treatment. Optionally, Ribavirin, a synthetic nucleoside analogue with activity against a broad spectrum of viruses including HCV, can be included in combination with the interferon and the HCV protease inhibitor. The recommended dosage of ribavirin is in a range from 600 to 1400 mg per day for at least 24 weeks (commercially available as REBETOL ribavirin from Schering-Plough or COPEGUS ribavirin from Hoffmann-La Roche).

In one embodiment, the compounds of the invention can be used to treat cellular proliferation diseases. Such cellular proliferation disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or subjects afflicted or subject to impending affliction with any one of these disorders or states.

The methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecoloqical: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-ydrazone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl) retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycarminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine, and mixture thereof.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino] benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide,N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kif14, inhibitors of Mphosph1 and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, neizarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin, simvastatin (ZOCOR®), pravastatin (PRAVACHOL®), fluvastatin and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401(1999).

Examples of farnesyl protein transferase inhibitors include SARASAR™(4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide from Schering-Plough Corporation, Kenilworth, N.J.), tipifarnib (Zarnestra® or R15777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α (for example Intron and Peg-Intron), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocrinol., Vol. 16, p. 107 (1996); Jpn. J. Pharrnacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mirieralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFla]) (see Thrombosis Res. 101:329-354 (2001)). Examples of TAFla inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5pyridinyl)pyridine; or a pharmaceutically acceptable salt thereof.

Compounds that have been described as specific inhibitors of COX-2 and are therefore useful in the present invention include, but are not limited to, parecoxib, CELIEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI1571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy)phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux and mixtures thereof.

Another embodiment of the present invention is the use of the present compounds in combination with gene therapy for the treatment of cancer. For an overview of genetic strategies to treating cancer, see Hall et al (Am J Hum Genet 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The present compounds can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drug, an inhibitor of cell proliferation and survival signaling, an agent that interfers with a cell cycle checkpoint, and an apoptosis inducing agent.

In one embodiment, the present invention emcompasses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention is a method of treating cancer comprising administering a therapeutically effective amount of at least one compound of the present invention in combination with radiation therapy and at least one compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interfers with a cell cycle checkpoint, and an apoptosis inducing agent.

Yet another embodiment of the invention is a method of treating cancer comprising administering a therapeutically effective amount of at least one compound of the present invention in combination with paclitaxel or trastuzumab.

The present invention also includes a pharmaceutical composition useful for treating or preventing the various disease states mentioned herein cellular proliferation diseases (such as cancer, hyperplasia, cardiac hypertrophy, autoimmune diseases, fungal disorders, arthritis, graft rejection, inflammatory bowel disease, immune disorders, inflammation, and cellular proliferation induced after medical procedures) that comprises a therapeutically effective amount of at least one compound of the present invention and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interfers with a cell cycle checkpoint, and an apoptosis inducing agent.

When the disease being treated by the cathepsin inhibitor compounds of the present invention is inflammatory disease, an embodiment of the present invention comprises administering: (a) a therapeutically effective amount of at least one compound of the present cathepsin inhibitors (e.g., a compound according to Formula I-XXVII) or a pharmaceutically acceptable salt, solvate or ester thereof concurrently or sequentially with (b) at least one medicament selected from the group consisting of: disease modifying antirheumatic drugs; nonsteroidal anti-inflammatory drugs; COX-2 selective inhibitors; COX-1 inhibitors; immunosuppressives (non-limiting examples include methotrexate, cyclosporin, FK506); steroids; PDE IV inhibitors, anti-TNF-α compounds, TNF-alpha-convertase inhibitors, cytokine inhibitors, MMP inhibitors, glucocorticoids, chemokine inhibitors, CB2-selective inhibitors, p38 inhibitors, biological response modifiers; anti-inflammatory agents and therapeutics.

Another embodiment of the present invention is directed to a method of inhibiting or blocking T-cell mediated chemotaxis in a patient in need of such treatment the method comprising administering to the patient a therapeutically effective amount of at least one compound of the present cathepsin inhibitors (e.g., a compound according to formula I-XXVII) or a pharmaceutically acceptable salt, solvate or ester thereof.

Another embodiment of this invention is directed to a method of treating inflammatory bowel disease in a patient in need of such treatment comprising administering to the patient a therapeutically effective amount of at least one compound according to the present cathepsin inhibitors or a pharmaceutically acceptable salt, solvate or ester thereof.

Another embodiment of this invention is directed to a method of treating or preventing graft rejection in a patient in need of such treatment comprising administering to the patient a therapeutically effective amount of at least one compound according to the present cathepsin inhibitors, or a pharmaceutically acceptable salt, solvate or ester thereof.

Another embodiment of this invention is directed to a method comprising administering to the patient a therapeutically effective amount of: (a) at least one compound according to the present cathepsin inhibitors, or a pharmaceutically acceptable salt, solvate or ester thereof concurrently or sequentially with (b) at least one compound selected from the group consisting of: cyclosporine A, FK-506, FTY720, beta-Interferon, rapamycin, mycophenolate, prednisolone, azathioprine, cyclophosphamide and an antilymphocyte globulin.

Another embodiment of this invention is directed to a method of treating multiple sclerosis in a patient in need of such treatment the method comprising administering to the patient a therapeutically effective amount of: (a) at least one compound according to the present cathepsin inhibitors, or a pharmaceutically acceptable salt, solvate or ester thereof concurrently or sequentially with (b) at least one compound selected from the group consisting of: beta-interferon, glatiramer acetate, glucocorticoids, methotrexate, azothioprine, mitoxantrone, VLA-4 inhibitors and/or CB2-selective inhibitors.

Another embodiment of this invention is directed to a method of treating multiple sclerosis in a patient in need of such treatment the method comprising administering to the patient a therapeutically effective amount of: a) at least one compound according to the present cathepsin inhibitors, or a pharmaceutically acceptable salt, solvate or ester thereof concurrently or sequentially with (b) at least one compound selected from the group consisting of: methotrexate, cyclosporin, leflunimide, sulfasalazine, β-methasone, β-interferon, glatiramer acetate, prednisone, etonercept, and infliximab.

Another embodiment of this invention is directed to a method of treating rheumatoid arthritis in a patient in need of such treatment the method comprising administering to the patient a therapeutically effective amount of: (a) at least one compound according to the present cathepsin inhibitors or a pharmaceutically acceptable salt, solvate or ester thereof concurrently or sequentially with (b) at least one compound selected from the group consisting of: COX-2 inhibitors, COX inhibitors, immunosuppressives, steroids, PDE IV inhibitors, anti-TNF-α compounds, MMP inhibitors, glucocorticoids, chemokine inhibitors, CB2-selective inhibitors, caspase (ICE) inhibitors and other classes of compounds indicated for the treatment of rheumatoid arthritis.

Another embodiment of this invention is directed to a method of treating psoriasis in a patient in need of such treatment the method comprising administering to the patient a therapeutically effective amount of: a) at least one compound according to present cathepsin inhibitors, or a pharmaceutically acceptable salt, solvate or ester thereof concurrently or sequentially with (b) at least one compound selected from the group consisting of: immunosuppressives, steroids, and anti-TNF-α compounds.

Another embodiment of this invention is directed to a method of treating a disease selected from the group consisting of: inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy, type I diabetes, viral meningitis and tumors in a patient in need of such treatment, such method comprising administering to the patient an effective amount of at least one compound according to present cathepsin inhibitors, or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy and cancer in a patient in need of such treatment, such method comprising administering to the patient an effective amount of at least one compound according to the present cathepsin inhibitors, or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses and tuberculoid leprosy, type I diabetes, viral meningitis and cancer in a patient in need of such treatment, such method comprising administering to the patient an effective amount of (a) at least one compound according to the present cathepsin inhibitors, or a pharmaceutically acceptable salt, solvate or ester thereof concurrently or sequentially with (b) at least one medicament selected from the group consisting of: disease modifying antirheumatic drugs; nonsteroidal anti-inflammatory drugs; COX-2 selective inhibitors; COX-1 inhibitors; immunosuppressives; steroids; PDE IV inhibitors, anti-TNF-α compounds, MMP inhibitors, glucocorticoids, chemokine inhibitors, CB2-selective inhibitors, biological response modifiers; anti-inflammatory agents and therapeutics.

When the present invention involves a method of treating a cardiovascular disease, in addition to administering the cathepsin inhibitors of the present invention, the method further comprises administering to the subject in need one or more pharmacological or therapeutic agents or drugs such as cholesterol biosynthesis inhibitors and/or lipid-lowering agents discussed below.

Non-limiting examples of cholesterol biosynthesis inhibitors for use in the compositions, therapeutic combinations and methods of the present invention include competitive inhibitors of HMG CoA reductase, the rate-limiting step in cholesterol biosynthesis, squalene synthase inhibitors, squalene epoxidase inhibitors and mixtures thereof. Non-limiting examples of suitable HMG CoA reductase inhibitors include statins such as lovastatin (for example MEVACOR® which is available from Merck & Co.), pravastatin (for example PRAVACHOL® which is available from Bristol Meyers Squibb), fluvastatin, simvastatin (for example ZOCOR® which is available from Merck & Co.), atorvastatin, cerivastatin, rosuvastatin, rivastatin (sodium 7-(4-fluorophenyl)-2,6-diisopropyl-5-methoxymethylpyridin-3-yl)-3,5-dihydroxy-6-heptanoate, CI-981 and pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Preferred HMG CoA reductase inhibitors include lovastatin, pravastatin and simvastatin.

In another embodiment, the method of treatment comprises administering the present cathepsin inhibitors in combination with one or more cardiovascular agents and one or more cholesterol biosynthesis inhibitors.

In another alternative embodiment, the method treatment of the present invention can further comprise administering nicotinic acid (niacin) and/or derivatives thereof coadministered with or in combination with the cardiovascular agent(s) and sterol absorption inhibitor(s) discussed above.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include niceritrol, nicofuranose and acipimox (5-methyl pyrazine-2-carboxylic acid 4-oxide). Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

In another alternative embodiment, the method of treatment of the present invention can further comprise administering one or more AcylCoA:Cholesterol O-acyltransferase (“ACAT”) Inhibitors, which can reduce LDL and VLDL levels, coadministered with or in combination with the cardiovascular agent(s) and sterol absorption inhibitor(s) discussed above. ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 2000 July; 60(1); 55-93, which is incorporated by reference herein.

In another alternative embodiment, the method of treatment of the present invention can further comprise administering probucol or derivatives thereof (such as AGI-1067 and other derivatives disclosed in U.S. Pat. Nos. 6,121,319 and 6,147,250), which can reduce LDL levels, coadministered with or in combination with the cardiovascular agent(s) and sterol absorption inhibitor(s) discussed above.

In another alternative embodiment, the method of treatment of the present invention can further comprise administering fish oil, which contains Omega 3 fatty acids (3-PUFA), which can reduce VLDL and triglyceride levels, coadministered with or in combination with the cardiovascular agent(s) and sterol absorption inhibitor(s) discussed above. Generally, a total daily dosage of fish oil or Omega 3 fatty acids can range from about 1 to about 30 grams per day in single or 2-4 divided doses.

In another alternative embodiment, the method of treatment of the present invention can further comprise administering natural water soluble fibers, such as psyllium, guar, oat and pectin, which can reduce cholesterol levels, coadministered with or in combination with the cardiovascular agent(s) and sterol absorption inhibitor(s) discussed above. Generally, a total daily dosage of natural water soluble fibers can range from about 0.1 to about 10 grams per day in single or 2-4 divided doses.

In another alternative embodiment, the method of treatment of the present invention can further comprise administering plant sterols, plant stanols and/or fatty acid esters of plant stanols, such as sitostanol ester used in BENECOL® margarine, which can reduce cholesterol levels, coadministered with or in combination with the cardiovascular agent(s) and sterol absorption inhibitor(s) discussed above. Generally, a total daily dosage of plant sterols, plant stanols and/or fatty acid esters of plant stanols can range from about 0.5 to about 20 grams per day in single or 2-4 divided doses.

In another alternative embodiment, the method of treatment of the present invention can further comprise administering antioxidants, such as probucol, tocopherol, ascorbic acid, β-carotene and selenium, or vitamins such as vitamin B₆ or vitamin B₁₂, coadministered with or in combination with the cardiovascular agent(s) and sterol absorption inhibitor(s) discussed above. Generally, a total daily dosage of antioxidants or vitamins can range from about 0.05 to about 10 grams per day in single or 2-4 divided doses.

In another alternative embodiment, the method of treatment of the present invention can further comprise administering one or more bile acid sequestrants (insoluble anion exchange resins), coadministered with or in combination with the cardiovascular agents and sterol absorption inhibitor(s) discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors which bind LDL from plasma to further reduce cholesterol levels in the blood.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

Also useful with the present invention are methods of treatment that can further comprise administering at least one (one or more) activators for peroxisome proliferator-activated receptors (PPAR). These activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARβ and as NUC1, and each of these names refers to the same receptor.

PPARα regulates the metabolism of lipids. PPARα is activated by fibrates and a number of medium and long-chain fatty acids, and it is involved in stimulating β-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include the fibrates discussed above.

Other examples of PPARα activators useful with the practice of the present invention include suitable fluorophenyl compounds as disclosed in U.S. Pat. No. 6,028,109 which is incorporated herein by reference; certain substituted phenylpropionic compounds as disclosed in WO 00/75103 which is incorporated herein by reference; and PPARα activator compounds as disclosed in WO 98/43081 which is incorporated herein by reference.

Non-limiting examples of PPARγ activator include suitable derivatives of glitazones or thiazolidinediones, such as, troglitazone (such as REZULIN® troglitazone (-5-[[4-[3,4-dihydro-6-hydroxy-2,5,7,8-tetramethyl-2H-1-benzopyran-2-yl)methoxy]phenyl]methyl]-2,4-thiazolidinedione)commercially available from Parke-Davis); rosiglitazone (such as AVANDIA® rosiglitazone maleate (-5-[[4-[2-(methyl-2-pyridinylamino)ethoxy]phenyl]methyl]-2,4-thiazolidinedione, (Z)-2-butenedioate) (1:1) commercially available from SmithKline Beecham) and pioglitazone (such as ACTOS™ pioglitazone hydrochloride (5-[[4-[2-(5-ethyl-2-pyridinyl)ethoxy]phenyl]methyl]-2,4-]thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARγ activator compounds disclosed in U.S. Pat. No. 5,994,554 which is incorporated herein by reference.

Other useful classes of PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 & WO 01/12187 which are incorporated herein by reference; and substituted 4-hydroxy-phenylalconic acid compounds as disclosed in WO 97/31907 which is incorporated herein by reference.

PPARδ compounds are useful for, among other things, lowering triglyceride levels or raising HDL levels. Non-limiting examples of PPARδ activators include suitable thiazole and oxazole derivates, such as C.A.S. Registry No. 317318-32-4, as disclosed in WO 01/00603 which is incorporated herein by reference); certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-[difluorophenyl]-1heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; o-anisamide compounds as disclosed in WO 01/21578 which is incorporated herein by reference; and PPAR activator compounds as disclosed in WO 01/40192 which is incorporated herein by reference.

Also useful with the present invention are methods of treatment which further comprise administering hormone replacement agents and compositions. Useful hormone agents and compositions for hormone replacement therapy of the present invention include androgens, estrogens, progestins, their pharmaceutically acceptable salts and derivatives. Combinations of these agents and compositions are also useful.

The cathepsin inhibitors of the present invention are useful in the treatment of central nervous system diseases such as depression, cognitive function diseases and neurodegenerative diseases such as Parkinson's disease, senile dementia as in Alzheimer's disease, and psychoses of organic origin. In particular, the cathepsin inhibitors of the present invention can improve motor-impairment due to neurodegenerative diseases such as Parkinson's disease.

The other agents known to be useful in the treatment of Parkinson's disease which can be administered in combination with the cathepsin inhibitors of the present invention include: L-DOPA; dopaminergic agonists such as quinpirole, ropinirole, pramipexole, pergolide and bromocriptine; MAO-B inhibitors such as deprenyl and selegiline; DOPA decarboxylase inhibitors such as carbidopa and benserazide; and COMT inhibitors such as tolcapone and entacapone.

A preferred dosage for the administration of a compound of the present invention is about 0.001 to 500 mg/kg of body weight/day of a compound of the present invention or a pharmaceutically acceptable salt or ester thereof. An especially preferred dosage is about 0.01 to 25 mg/kg of body weight/day of a compound of the present invention or a pharmaceutically acceptable salt or ester thereof.

The phrases “effective amount” and “therapeutically effective amount” mean that amount of a compound of the present invention, and other pharmacological or therapeutic agents described herein, that will elicit a biological or medical response of a tissue, a system, or a subject (e.g., animal or human) that is being sought by the administrator (such as a researcher, doctor or veterinarian) which includes alleviation of the symptoms of the condition or disease being treated and the prevention, slowing or halting of progression of one or more of the presently claimed diseases. The formulations or compositions, combinations and treatments of the present invention can be administered by any suitable means which produce contact of these compounds with the site of action in the body of, for example, a mammal or human.

For administration of pharmaceutically acceptable salts of the above compounds, the weights indicated above refer to the weight of the acid equivalent or the base equivalent of the therapeutic compound derived from the salt.

As described above, this invention includes combinations comprising an amount of at least one compound of the presently claimed methods or a pharmaceutically acceptable salt or ester thereof, and an amount of one or more additional therapeutic agents listed above (administered together or sequentially) wherein the amounts of the compounds/ treatments result in desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of the present invention and an additional therapeutic agent may be present in fixed amounts (dosage amounts) in a single dosage unit (e.g., a capsule, a tablet and the like).

If formulated as a fixed dose, such combination products employ the compounds of this invention within the dosage range described herein and the other pharmaceutically active agent or treatment within its dosage range. Compounds of the present invention may also be administered sequentially with known therapeutic agents when a combination formulation is inappropriate. The invention is not limited in the sequence of administration; compounds of the present invention may be administered either prior to or after administration of the known therapeutic agent. Such techniques are within the skills of persons skilled in the art as well as attending physicians.

The pharmacological properties of the compounds of this invention may be confirmed by a number of pharmacological assays for measuring HCV viral activity or cathepsin activity, such as are well know to those skilled in the art.

The compositions of the present invention comprise at least one compound of Formulae I to XXVI, as defined above, optionally together with one or more acceptable controlled-release carriers, other adjuvants or vehicles thereof and optionally other therapeutic agents. Each carrier, adjuvant or vehicle must be acceptable in the sense of being compatible with the other ingredients of the composition and not injurious to the mammal in need of treatment.

The compositions of the present invention are formulated and/or administered in such a way to maintain an average Cmin plasma concentration of at least of the compounds of Formulae I to XXVI at or above 10 ng/ml. However, in other embodiments, the average Cmin plasma concentration of the at least one protease inhibitor may be maintained at or above 50 ng/ml, 100 ng/ml, 150 ng/ml or 200 ng, ml. Cmin is generally defined as the minimum concentration of drug in plasma to obtain a predetermined intensity of response. Cmin is a measure of the concentration of drug in blood/plasma and is typically quantified at a time when the drug concentration will be near its lowest level, i.e. before the next predetermined dose of the drug. In vitro studies are typically performed to measure the concentration of a drug needed to inhibit viral replication by 50%, 90% or 95%. These values are known as the IC₅₀, IC₉₀ and IC₉₅, respectively. Generally IC values refer to the concentration of active agent required to inhibit a target virus. Without intending to be bound by theory, it is believed that a minimum average plasma concentration (Cmin) of 10 ng/ml of at least one of the compounds of Formula I to XXVI correlates to at least IC₅₀. Further, it is believed that an average Cmin of 200 ng/ml correlates to at least IC₉₀.

The compositions of the present invention may be formulated with one or more controlled-release carriers to provide the rate controlled-release of any one or more of the components or active ingredients to optimize the therapeutic effects, i.e. HCV inhibitory activity and the like. Suitable dosage formulations for sustained release include, inter alia, layered tablets containing layers of varying disintegration rates or controlled release polymeric matrices impregnated with the active components and shaped in tablet form or capsules containing such impregnated or encapsulated porous polymeric matrices.

Controlled-release is a term known in the medicinal art and is typically used interchangeably with delayed release, slow release, controlled availability, slow acting, extended release, and metered release. Controlled-release is generally defined as the release of an agent from a dosage formulation slowly over a period of time, such as over hours or days. In the present invention, controlled-release is further defined as administering a predetermined dose of at least one of the compounds of Formulae I to XXVI over a predetermined period of time.

As discussed above, the present invention discloses dosage formulations and methods of using the same in which a predetermined dose of at least one of the compounds of Formulae I to XXVI are administered to maintain the average Cmin plasma concentration of the at least one HCV protease inhibitor at or above about 10 ng/ml. However, the average Cmin plasma concentration of the at least one protease inhibitor may be at or above 50 ng/ml, 100 ng/ml, 150 ng/ml or 200 ng/ml. Cmin is generally defined as the minimum concentration of drug in plasma to obtain a predetermined intensity of response. The dosage formulation and method are intended to treat, prevent, and/or ameliorate disorders associated with HCV. The dosage formulation and method are further intended to treat and/or reduce the signs and/or symptoms associated with HCV.

The rate of dissolution of the formulation can range from_to about_mg per hour, preferably from_to about_mg per hour. [Kaly, please provide ranges]. Dissolution can be determined according to standard USP procedures well known to those skilled in the art. A non-limiting example of a suitable procedure for determining dissolution is described in the following table:

(50 mg) Dissolution Procedure

| | |
|---|---|
| Apparatus | USP Apparatus 2 (Paddles) |
| Dissolution | 0.5% SDS in phosphate buffer, pH 6.8, 500 mL for 50 mg |
| Medium | strength |
| Temperature | 37° C. |
| Detection | HPLC with UV detector at 220 nm wavelength |

The dosage formulation has at least one dosage unit, but may contain a plurality of dosage units, ranging from 2-100 dosage units. An oral dosage formulation may be provided, such as one of the following: tablets, capsules, or caplets. A transdermal treatment via a medicated patch may also be used as the controlled-release dosage formulation.

The dosage formulation contains from about 1 mg to about 3000 mg of at least one compound from Formulae I to XXVI discussed herein. The dosage formulation may be administered once a day, twice a day, three times a day, four times a day, or more frequently. In one non-limiting embodiment, 400 mg of HCV protease inhibitor is administered three times a day. However, the dosing schedule may be at from about 100 mg a day, 100 mg twice a day, 200 mg twice a day, 400 mg twice a day, 600 mg twice a day, or 600 mg three times a day. Also, as discussed herein, the amount and frequency of administration of the formulations of the present invention will be regulated according to the judgment of the attending clinician considering such factors as age, condition and size of the patient as well as severity of the symptoms being treated. A typical recommended daily dosage regimen for oral administration can range from about 50 mg/day to about 3000 mg/day, in two to four divided doses.

The quantity of active compound in a unit dose of preparation may be varied or adjusted from about 1 mg to about 1000 mg, or from about 50 mg to about 800 mg, or from about 50 mg to about 600 mg, or from about 50 mg to about 400 mg, or from about 50 mg to about 200 mg according to the particular application. In one embodiment, the dosage formulation contains about 200 mg of the active compound.

The dosage formulation may be administered at a time of day to coincide with the circadian rhythm of the subject being treated. Circadian rhythms are endogenous oscillations that occur with a periodicity of about 24 hours, and are synchronized according to internal biologic clocks related to the sleep-wake cycle. The dosage formulation thus may be administered in one or more discrete dosages over a twenty-four hour time interval in an asymmetric pattern as to dosage amount and/or timing of dosage, wherein the at least one compound is selected from the group consisting of compounds of Formulae I-XXVI, as described above.

As used herein, the term “asymmetric pattern” as to dosing means that at least one dose administered in a 24-hour period differs from the other doses administered in the same time interval in the amount of active ingredient in the dose. The term “asymmetric pattern” as to timing of dosage means that at least one dose in a 24-hour period will be administered after a longer or shorter interval than interval between the previous two doses.

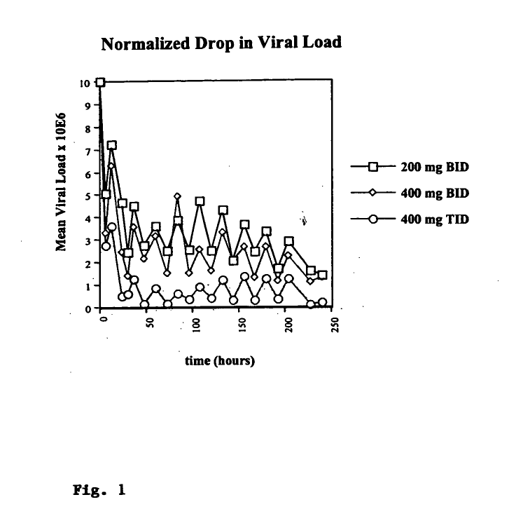

Studies of viral activity in HCV infected patients indicate that viral activity and resulting viral load are influenced by the circadian rhythm of the patient. As shown in FIG. 1, in patients treated with compounds of the present invention the decline in viral load seen with treatment is cyclical, with the viral load declining during cell division in the liver, and increasing at times when no cell division is occurring. During cell division the virus is unable to replicate, and the viral load declines. Thus, in one aspect of the invention, the one or more discrete dosages are adjusted in amount to provide a highest dose or doses at a time or times corresponding to the time interval when replication of the hepatitis-C virus is highest.

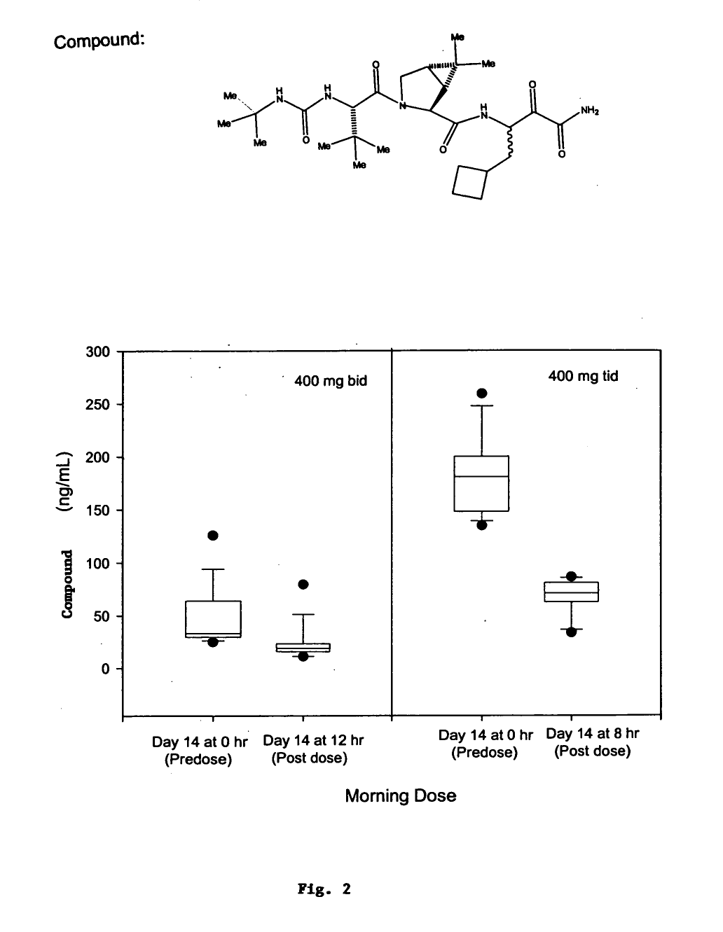

It has been determined that metabolism of compounds of the present invention is also affected by the patient's circadian rhythm. As shown in FIG. 2 (right hand box), plasma levels of the drug are highest in the morning, when measured 8 hours after the previous dose and before the morning dose is administered. Plasma levels 8 hours after the morning dose are much lower, suggesting that metabolism of the drug is faster during the day than at night.

Accordingly, in another aspect of the present invention, the one or more discrete dosages are adjusted in amount to provide a highest dose or doses at a time or times corresponding to the time interval when metabolism of the protease inhibitor is highest. In a preferred embodiment, the one or more discrete dosages is three doses, administered as one dose of 300 mg, one dose of 400 mg, and one dose of 500 mg, each dose administered every 8 hours, wherein the 500 mg dose is administered at a time corresponding to the time interval of highest replication of the hepatitis-C virus and/or highest metabolism of the protease inhibitor. It may also be desirable to provide different patterns of dosage, such as, but not limited to 200, 300, 700; or 200, 200, 300, 500; 200, 200, 200, 600, or other combinations, depending on considerations such as the length of time that highest viral replication is occurring, metabolism of the protease inhibitor and the highest tolerated dose. One skilled in the art can determine the appropriate number of doses and dose amounts without undue experimentation.

Alternatively, and in additional embodiments, the one or more discrete dosages is administered in equal dose amounts but staggered as to timing of administration, to accommodate fluctuations in viral load and/or drug metabolism. For example, if the total desired dose over 24 hours is 1200 mg, it can be administered as a 300 mg/dose, at 8 am, 12 noon, 4 pm, and 8 pm, with a 12-hour interval between the evening dose and the morning dose. This example is non-limiting, and one skilled in the art can easily determine the appropriate number of doses and the timing of administration. In a preferred embodiment, the one or more discrete dosages is at least three doses in equal amounts, administered at unequal time intervals in twenty-four hours. The time intervals of dosage are adjusted to provide administration of one or more doses at a time or times corresponding to the time interval of highest replication of the hepatitis-C virus, or they can be adjusted to provide administration of one or more doses at a time or times corresponding to the time interval of highest metabolism of the protease inhibitor.

As will be understood by one skilled in the art, both the amount of dosage given over a 24-hour period and the timing of administration can be varied in an asymmetric pattern. The asymmetric pattern of dose amount or timing of dosage is adjusted to accommodate variations in viral replication and/or metabolism of the protease inhibitor influenced by the patient's circadian rhythm.

Further, as discussed herein, the dosage formulation may be administered concurrently or sequentially as combination therapy with at least one of an antiviral agent and/or at least one of an immunomodulatory agent that are different from the compounds disclosed in Formulae I to XXVI. Further, the different antiviral agent(s) and/or the immunomodulatory agent(s) may be contained within the dosage formulation with the compounds disclosed in Formulae I to XXVI. As discussed herein, the dosage formulation may contain at least one anti-cancer agent or may be administered concurrently or sequentially with at least one anti-cancer agent.

In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition.

In one embodiment, the dosage formulation has at least one dosage unit, but may contain a plurality of dosage units, ranging from 2-100 dosage units. An oral dosage formulation may be provided, such as one of the following: tablets, capsules, caplets, solutions, suspensions, emulsions, troches, lozenges, effervescent tablets, lollipops, and reconstitutible powders. Further, the dosage formulation may be an injectable dosage formulation for intravenous or subcutaneous administration. Alternatively, the dosage formulation may be an intranasal, aerosol, or suppository preparation. A transdermal preparation may be provided, such as one of the following: creams, lotions, aerosols, emulsions or medicated patches.

The dosage formulation contains from about 1 mg to about 3000 mg of at least one compound from Formulae I to XXVI discussed herein. The dosage formulation may be administered once a day, twice a day, three times a day, four times a day, or more frequently. In one non-limiting embodiment, 400 mg of the compound is administered three times a day. However, the dosing schedule may be at from about 100 mg a day, 100 mg twice a day, 200 mg twice a day, 400 mg twice a day, 600 mg twice a day, or 600 mg three times a day. Also, as discussed herein, the amount and frequency of administration of the formulations of the present invention will be regulated according to the judgment of the attending clinician considering such factors as age, condition and size of the patient as well as severity of the symptoms being treated. A typical recommended daily dosage regimen for oral administration can range from about 50 mg/day to about 3000 mg/day, in two to four divided doses.

The quantity of active compound in a unit dose of preparation may be varied or adjusted from about 1 mg to about 1000 mg, or from about 50 mg to about 800 mg, or from about 50 mg to about 600 mg, or from about 50 mg to about 400 mg, or from about 50 mg to about 200 mg according to the particular application. In one embodiment, the dosage formulation contains about 200 mg of the active compound.

Suitable controlled-release carrier forms include general types now known or heretofore developed in the art. Examples include and are incorporated herein by reference, but are not limited to, hydrophilic polymers as disclosed in U.S. Patent Application Publication No. 2004/0156899, multi-layer release beads as disclosed in U.S. Pat. No. 6,673,367, controlled-release beads as disclosed in U.S. Pat. No. 6,770,295, coated tablets as disclosed in U.S. Pat. Nos. 4,990,535 and 5,100,675, matrix core tablets as disclosed in U.S. Pat. No. 5,314,697, bilayer tablets as disclosed in WO 01/45676, controlled-release beads as disclosed in U.S. Pat. No. 6,630,162, and osmotic dosage formulations as disclosed in U.S. Pat. Nos. 4,777,049, 4,851,229, and 5,178,867.

In one non-limiting embodiment, the controlled-release carrier is a swellable polymer. The swellable polymer is a biocompatible or bioerodible, hydrophilic polymer, preferably a cellulosic polymer. The term “hydrophilic” is generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water. Hydrophilic polymeric carriers are thus compatible with aqueous fluids such as those present in the human body.

The term “polymer” as used herein refers to a molecule containing a plurality of covalently attached monomer units, and includes branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers.

The terms “swellable” and “bioerodible” (or simply “erodible”) are used to refer to the preferred polymers herein, with “swellable” polymers being those that are capable of absorbing water and physically swelling as a result, with the extent to which a polymer can swell being determined by the degree of crosslinking, and “bioerodible” or “erodible” polymers referring to polymers that slowly dissolve and/or gradually hydrolyze in an aqueous fluid, and/or that physically erodes as a result of movement within the stomach or gastrointestinal tract.

Polymers suitable for use in the present invention are those that both swell upon absorption of gastric fluid and gradually erode over a time period of hours. Erosion initiates simultaneously with the swelling process, upon contact of the surface of the dosage formulation with gastric fluid. Erosion reflects the dissolution of the polymer beyond the polymer gel-solution interface where the polymer has become sufficiently dilute that it can be transported away from the dosage formulation by diffusion or convection. This may also depend on the hydrodynamic and mechanical forces present in the gastrointestinal tract during the digestive process. While swelling and erosion occur at the same time, it is preferred herein that drug release should be erosion-controlled, meaning that the selected polymer should be such that complete drug release occurs primarily as a result of erosion rather than swelling and dissolution. However, swelling should take place at a rate that is sufficiently fast to allow the tablet to be retained in the stomach. At minimum, for an erosional gastric retentive dosage formulation, there should be an extended period during which the dosage formulation maintains its size before it is diminished by erosion.

Suitable polymers for use in the present dosage formulations may be linear, branched, dendrimeric, or star polymers, and include synthetic hydrophilic polymers as well as semi-synthetic and naturally occurring hydrophilic polymers. The polymers may be homopolymers or copolymers, if copolymers, either random copolymers, block copolymers or graft copolymers. Synthetic hydrophilic polymers useful herein include, but are not limited to:

polyalkylene oxides, particularly poly(ethylene oxide), polyethylene glycol and poly(ethylene oxide)-poly(propylene oxide) copolymers;

cellulosic polymers;

acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate;

maleic anhydride copolymers;

polymaleic acid;

poly(acrylamides) such as polyacrylamide per se, poly(methacrylamide), poly(dimethylacrylamide), and poly(N-isopropyl-acrylamide);

poly(olefinic alcohol) such as poly(vinyl alcohol);

poly(N-vinyl lactams) such as poly(vinyl pyrrolidone), poly(N-vinyl caprolactam), and copolymers thereof;

polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol;

polyoxyethylated sorbitol and polyoxyethylated glucose;

polyoxazolines, including poly(methyloxazoline) and poly(ethyloxazoline);

polyvinylamines;

polyvinylacetates, including polyvinylacetate per se as well as ethylene-vinyl acetate copolymers, polyvinyl acetate phthalate, and the like;

polyimines, such as polyethyleneimine;

starch and starch-based polymers;

polyurethane hydrogels;

chitosan;

polysaccharide gums;

zein; and

shellac, ammoniated shellac, shellac-acetyl alcohol, and shellac n-butyl stearate.

The term “cellulosic polymer” is used herein to denote a linear polymer of anhydroglucose. Cellulosic polymers that can be used advantageously in the present dosage formulations include, without limitation, hydroxymethylcellulose, hydroxypropylcellulose, hydroxyethylcellulose, hydroxypropyl methylcellulose, methylcellulose, ethylcellulose, cellulose acetate, cellulose acetate phthalate, cellulose acetate trimellitate, hydroxypropyl methylcellulose phthalate, hydroxypropylcellulose phthalate, cellulose hexahydrophthalate, cellulose acetate hexahydrophthalate, carboxymethylcellulose, carboxymethylcellulose sodium, and microcrystalline cellulose. Preferred cellulosic polymers are alkyl-substituted cellulosic polymers that ultimately dissolve in the GI tract in a predictably delayed manner. Preferred alkyl-substituted cellulose derivatives are those substituted with alkyl groups of 1 to 3 carbon atoms each. Examples are methylcellulose, hydroxymethylcellulose, hydroxyethylcellulose, hydroxypropylcellulose, hydroxypropyl methylcellulose, and carboxymethylcellulose and mixtures thereof. In terms of their viscosities, one class of preferred alkyl-substituted celluloses includes those whose viscosity is within the range of about 50 to about 110,000 centipoise as a 2% aqueous solution at 20° C. Another class includes those whose viscosity is within the range of about 800 to about 6,000 centipoise as a 1% aqueous solution at 20° C. Particularly preferred alkyl-substituted celluloses are hydroxyethylcellulose and hydroxypropylmethylcellulose. A presently preferred hydroxyethylcellulose is NATRASOL® 250HX NF (National Formulary), available from Aqualon Company, Wilmington, Del., USA.

Suitable polymers also include naturally occurring hydrophilic polymers such as, by way of example, proteins such as collagen, fibronectin, albumins, globulins, fibrinogen, fibrin and thrombin; aminated polysaccharides, particularly the glycosaminoglycans, e.g., hyaluronic acid, chitin, chondroitin sulfate A, B, or C, keratin sulfate, keratosulfate and heparin; guar gum; xanthan gum; carageenan; alginates; pectin; and activated polysaccharides such as dextran and starches.

The aforementioned list of polymers is not exhaustive, and a variety of other synthetic hydrophilic polymers may be used, as will be appreciated by those skilled in the art.

The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water. Biodegradable segments may be composed of small molecular segments such as ester linkages, anhydride linkages, ortho ester linkages, ortho carbonate linkages, amide linkages, phosphonate linkages, etc.

Any polymer or polymers of the matrix may also be crosslinked, with the degree of crosslinking directly affecting the rate of polymer swelling as well as the erosion rate. That is, a polymer having a higher degree of crosslinking will exhibit less swelling and slower erosion than a polymer having a lower degree of crosslinking. Crosslinked polymers may be prepared using the above-mentioned exemplary polymers using conventional crosslinking procedures (e.g., chemical crosslinking with an added crosslinking agent, photolytically induced crosslinking, etc.), or the polymers may be obtained commercially in crosslinked form.

The water-swellable polymers can be used individually or in combination. Certain combinations will often provide a more controlled release of the drug than their components when used individually. Examples include, but are not limited to, the following: a cellulosic polymer combined with a gum, such as hydroxyethylcellulose or hydroxypropylcellulose combined with xanthan gum; a polyalkylene oxide combined with a gum, such as poly(ethylene oxide) combined with xanthan gum; and a polyalkylene oxide combined with a cellulosic polymer, such as poly(ethylene oxide) combined with hydroxyethylcellulose or hydroxypropylcellulose.

Combinations of different poly(ethylene oxide)s are also contemplated, with polymers of different molecular weights contributing to different dosage formulation characteristics. For example, a very high molecular weight poly(ethylene oxide) such as Polyox® 303 (with a number average molecular weight of 7 million) or Polyox® Coag (with a number average molecular weight of 5 million) may be used to significantly enhance diffusion relative to disintegration release by providing high swelling as well as tablet integrity. Incorporating a lower molecular weight poly(ethylene oxide) such as Polyox® WSR N-60K (number average molecular weight approximately 2 million) with Polyox® 303 and/or Polyox® Coag increases disintegration rate relative to diffusion rate, as the lower molecular weight polymer reduces swelling and acts as an effective tablet disintegrant. Incorporating an even lower molecular weight poly(ethylene oxide) such as Polyox® WSR N-80 (number average molecular weight approximately 200,000) further increases disintegration rate.

The hydrophilicity and water swellability of these polymers cause the drug-containing matrices to swell in size in the gastric cavity due to ingress of water in order to achieve a size that will be retained in the stomach when introduced during the fed mode. These qualities also cause the matrices to become slippery, which provides resistance to peristalsis and further promotes their retention in the stomach. The release rate of a drug from the matrix is primarily dependent upon the rate of water imbibition and the rate at which the drug dissolves and diffuses from the swollen polymer, which in turn is related to the solubility and dissolution rate of the drug, the drug particle size and the drug concentration in the matrix.

The amount of polymer relative to the drug can vary, depending on the drug release rate desired and on the polymer, its molecular weight, and excipients that may be present in the formulation. The amount of polymer will be sufficient however to retain at least about 40% of the drug within the matrix one hour after ingestion (or immersion in the gastric fluid). Preferably, the amount of polymer is such that at least 50% of the drug remains in the matrix one hour after ingestion. More preferably, at least 60%, and most preferably at least 80%, of the drug remains in the matrix one hour after ingestion. In all cases, however, substantially all of the drug will be released from the matrix within about eight hours, and preferably within about six hours, after ingestion, “substantially all” meaning at least 85%, preferably at least 90%.

Higher molecular weight polymers may be preferred to provide a desired extended release profile using the present dosage formations. Suitable molecular weights are generally in the range of about 5,000 to about 20,000,000. For sparingly soluble drugs, the polymers have molecular weights preferably in the range of about 5,000 to about 8,000,000, more preferably in the range of about 10,000 to about 5,000,000. For water-soluble drugs, the polymers preferably have molecular weights of at least about 10,000, but the molecular weight used will vary with the selected polymer. For example, for hydroxypropyl methylcellulose, the minimum molecular weight may be as low as 10,000, while for poly(ethylene oxide)s the molecular weight may be far higher, on the order of 2,000,000 or more.

The swellable polymer used as the controlled-release dosage formation carrier is preferably present in an amount to obtain a weight gain level of the dosage formation from about 1 to 90 percent, or about 2 to 50 percent, or more preferably about 2 to 25 percent. The swellable polymer used as the controlled-release dosage formation carrier is also preferably present at from about 1 to 99 weight percent (wt. %), or about 2 to 98 weight percent (wt. %), or more preferably about 20 to 90 weight percent (wt. %).

The formulations of the present invention comprise at least one HCV protease inhibitor, as defined above, together with one or more pharmaceutically acceptable adjuvants and optionally other therapeutic agents and pharmaceutically acceptable carriers and excipients. Each excipient must be acceptable in the sense of being compatible with the other ingredients of the formulation and not injurious to the mammal in need of treatment.

In yet another embodiment, the present invention discloses methods for preparing the pharmaceutical formulations of the present invention. In the pharmaceutical formulations, the HCV protease inhibitor will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Powders and tablets may be comprised of from about 5 to about 95 percent of the HCV protease inhibitor.

In one embodiment, the adjuvant is at least one pharmaceutically acceptable surfactant or at least one acidifying agent or both. When desired or needed, suitable carriers and other excipients (such as binders, glidents, lubricants, and disintegrants) may also be incorporated in the formulation. These adjuvants, carriers and excipients as well as others are described hereinafter.

Surfactant refers to an adjuvant material that reduces the contact angle of the active drug component and may also be referred to as a wetting agent. Typically, the present HCV protease inhibitors have relatively low solubilities in aqueous systems (as in a mammalian body), such as less than 1 mg/ml. For example, the solubility of a compound of Formula 1a in water is about 0.6 mg/ml. Treatment of diseases requiring high dosages of the present compounds, such as HCV, is enhanced by improving the absorption rate of the compounds thereby improving the extent of absorption of the compounds in a mammal. The surfactant in the pharmaceutical formulations of the present invention enhances wetting of the present compounds and improves the dissolution rate of the compounds to render a greater quantity of the compounds available for absorption than is available in a formulation of the present compounds that does not include a surfactant. Any pharmaceutically acceptable surfactant that improves wetting of the present compounds may be used. Particularly suitable surfactants include sodium lauryl sulfate, stearic acid, monoethanolamine, docusate sodium, sorbitan fatty acid esters, polyoxyethylene sorbitan fatty acid esters, ethoxylated aliphatic alcohols, propylene glycol monocaprylate, glycerol monostearate, medium chain triglycerides, polyoxyethylene alkyl ethers, and polyoxyethylene stearates. In one embodiment, the surfactant is sodium lauryl sulfate. In another embodiment, the surfactant is a polyoxyethylene sorbitan fatty acid ester. In yet another embodiment, the surfactant is PEG-1-PEG-9-lauryl glycol ether. These surfactants may be used alone in or combination in the pharmaceutical formulations of the present invention in a total amount of about 0.1 to about 10% by weight or about 1 to about 5% by weight.

Acidifying agent refers to an adjuvant material that lowers the pH of the formulation. The present compounds are known to generally be most stable at acidic pH. Any pharmaceutically acceptable acidifying agent that improves wetting of the present compounds may be used. Particularly suitable acidifying agents include tartaric acid, ascorbic acid, citric acid, malic acid and succinic acid. In one embodiment, the acidifying agent is tartaric acid. These acidifying agents may be used alone in or combination in the pharmaceutical formulations of the present invention in a total amount of about 0.1 to about 10% by weight or about 1 to 5% by weight.

Carrier refers to a substance that usually makes up the major portion of the composition or dosage formation. Suitable carriers include celluloses such as microcrystalline cellulose; sugars such as lactose, sucrose, mannitol and sorbitol; and starches derived from wheat, corn, rice and potato. The amount of carrier in the formulation can range from about 10 to about 90% by weight of the total formulation, or about 25 to about 75% by weight, or about 30 to about 60% by weight, or about 12 to about 60% by weight. In one embodiment, the carrier is microcrystalline cellulose.

Binders refers to substances that bind or “glue” powders together and make them cohesive by forming granules, thus serving as the “adhesive” in the formulation. Binders add cohesive strength already available in the diluent or bulking agent. Suitable binders include sugars such as lactose, sucrose and corn sweeteners; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; polyethylene glycol; waxes and inorganics such as magnesium aluminum silicate. The amount of binder in the formulation can range from about 10 to about 90% by weight of the total formulation, or about 25 to about 75% by weight, or about 30 to about 60% by weight, or about 12 to about 60% by weight. In one embodiment, the binder is anhydrous lactose.

Glidents refers to material that prevents caking and improves the flow characteristics of granulations, so that flow is smooth and uniform. Suitable glidents include silicon dioxide and talc. The amount of glident in the formulation can range from about 0.1% to about 5% by weight of the total formulation, or from about 0.5 to about 3% by weight.

Lubricants are substances added to the dosage formation to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as boric acid sodium chloride, sodium benzoate, sodium acetate, sodium chloride sodium oleate, polyethylene glycols and d′l-leucine. Lubricants are usually added at the very last step before compression, since they must be present on the surfaces of the granules and in between them and the parts of the tablet press. The amount of lubricant in the formulation can range from about 0.1 to about 10% by weight of the formulation, or from about 0.5 to about 5% by weight.

Disintegrant refers to materials added to the formulation to help it break apart (disintegrate) and release the drug. Suitable disintegrants include starches; “cold water soluble” modified starches such as sodium carboxymethyl starch; natural and synthetic gums such as locust bean, karaya, guar gum, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the formulation, or from about 2 to about 10% by weight.

Coloring agents provide coloration to the formulation or the dosage formation. Such excipients can include food grade dyes and food grade dyes adsorbed onto a suitable adsorbent such as clay or aluminum oxide. The amount of the coloring agent can vary from about 0.1 to about 5% by weight of the formulation, or from about 0.1 to about 1%.

Sweetening agents, flavoring agents, stabilizers, antioxidants and preservatives may also be included where appropriate.

The term pharmaceutical formulation encompasses both the bulk formulation and individual unit dosage formations. The bulk composition is material that has not yet been formed into individual dosage units. An illustrative dosage unit is an oral dosage unit such as tablets, capsules and the like.

The formulations of the present invention may be administered orally or transdermally. Preferably, the pharmaceutical formulation is in a unit dosage formulation. In such form, the preparation is subdivided into suitably sized unit doses containing appropriate quantities of the active components, e.g., an effective amount to achieve the desired purpose. Suitable unit dosage formulations are solids, gels, or fluids. Solid form preparations include powders, tablets, dispersible granules, capsules, cachets and suppositories.

The powders, tablets and capsules may be comprised of from about 5 to about 95 percent active ingredient. Tablets, powders, cachets and capsules can be used as solid dosage formulations suitable for oral administration. Other examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Capsules are special containers or enclosures, often made of methyl cellulose, polyvinyl alcohols, or denatured gelatins or starch for holding or containing the pharmaceutical formulation. Hard shell capsules are typically made of blends of relatively high gel strength bone and pork skin gelatins. The capsule itself may contain small amounts of dyes, opaquing agents, plasticizers and preservatives.

Tablet refers to a compressed or molded solid dosage formulation containing the pharmaceutical formulation. The tablet can be prepared by compression of mixtures or granulations obtained by wet granulation, dry granulation or by compaction.

A gel, such as an oral gel refers to the formulations dispersed or solubilized in a hydrophillic semi-solid matrix.

Suppositories containing the formulations of the present invention may be prepared by melting a low melting wax such as a mixture of fatty acid glycerides such as cocoa butter, and dispersing the components of the formulations homogeneously therein by stirring or similar mixing. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize the therapeutic effects, i.e. HCV inhibitory activity and the like. Suitable dosage formulations for sustained release include layered tablets containing layers of varying disintegration rates or controlled release polymeric matrices impregnated with the active components and shaped in tablet form or capsules containing such impregnated or encapsulated porous polymeric matrices.

Fluid forms may be liquids including solutions, suspensions and emulsions containing the formulations. Non-limiting examples include water or water-propylene glycol solutions for parenteral injections or addition of sweeteners and pacifiers for oral solutions, suspensions and emulsions. Liquid form preparations may also include solutions for intranasal administration.

Also included are aerosol preparations of the present invention that are suitable for inhalation. Aerosols may include solutions and solids in powder form, which may be in combination with a pharmaceutically acceptable carrier such as inert compressed gas, e.g. nitrogen.

Also included are solid form preparations which are intended to be converted, shortly before use, to liquid form preparations for either oral or parenteral administration. Such liquid forms include solutions, suspensions and emulsions. Alternatively, the formulations of the present invention may be prepared in powder blends that can be suspended in water or juices.

Transdermal formulations may take the form of creams, lotions, aerosols and/or emulsions and can be included in a transdermal patch of the matrix or reservoir type as are conventional in the art for this purpose.

Bioavailability refers to the rate and extent to which the active drug ingredient or therapeutic moiety is absorbed into the systemic circulation from an administered dosage formulation as compared to a standard or control.

Conventional methods for preparing tablets and capsules are known. Such methods include dry methods such as direct compression and compression of granulation produced by compaction, or wet methods or other special procedures. In one embodiment, a capsule containing the pharmaceutical formulation of the present invention is produced by blending the active drug component with some excipients, compacting the mixing such as with a roller compactor, milling the compact, blending the milled material with any remaining excipients and filling the final blend into capsules.

In one embodiment, the pharmaceutical formulation of the present is administered orally and is in a unit dosage formulation. In such form, the preparation is subdivided into suitably sized unit doses containing appropriate quantities of the active component, e.g., an effective amount to achieve the desired purpose.

The amount and frequency of administration of the formulations of the present invention will be regulated according to the judgment of the attending clinician considering such factors as age, condition and size of the patient as well as severity of the symptoms being treated. A typical recommended daily dosage regimen for oral administration can range from about 50 mg/day to about 3000 mg/day, in two to four divided doses.

The quantity of active compound in a unit dose of preparation may be varied or adjusted from about 1 mg to about 1000 mg, or from about 50 mg to about 800 mg, or from about 50 mg to about 600 mg, or from about 50 mg to about 400 mg, or from about 50 mg to about 200 mg according to the particular application. In one embodiment, the dosage formulation contains about 200 mg of the active compound.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The following formulation exemplifies some of the dosage formulations of the present invention. In the formulation, the “Active Compound” designates any of the compounds of Formulae I-XXVI, as defined above, or a pharmaceutically acceptable sale, solvate or ester thereof.

### HYPOTHETICAL EXAMPLE

Tablet

| Ingredient | Amount | |
|---|---|---|
| Active Compound | 200-500 | mg |
| Swellable Polymer | 2-75% | |
| Microcrystalline cellulose | 0-60 | wt. % |
| Lactose | 0-60 | wt. % |
| Sodium lauryl sulfate | 0-10 | wt. % |
| Tartaric acid | 0-10 | wt. % |
| Silicon dioxide | 0-3 | wt. % |
| Magnesium stearate | 1-10 | wt. % |
| TOTAL TABLET WEIGHT | 300-1000 | mg |

The powdery Active Compound is blended with some of the ingredients and compacted with a roller compactor to densify the powder. The resulting compact is milled, blended with the remaining ingredients and filled into the capsule.

### EXAMPLE

In one treatment study, forty-five HCV genotype-1 patients who had previously failed to respond to pegylated interferon treatment (HCV-RNA decrease <2 log 10 after 12 weeks of treatment) received oral dosing of the compound of Formula Ia at 100 mg BID, 200 mg BID, 400 mg BID, or 400 mg TID for 14 days. The preliminary mean pharmacokinetic results for Cmin (ng/ml) are shown in Table 1 below:

TABLE 1
Plasma Mean Cmin (ng/ml) at 0 hour (trough) predose

| 100 mg BID | 200 mg BID | 400 mg BID | 400 mg TID |
|---|---|---|---|
| (n = 12) | (n = 12) | (n = 11) | (n = 10) |
| Cmin = 12.7 | Cmin = 46.3 | Cmin = 48.8 | Cmin = 183 |
| ng/ml | ng/ml | ng/ml | ng/ml |

Thus, as shown in Table 1, the dosage formulation and method of administering the compound of Formula la was able to maintain an average Cmin plasma concentration at or above 10 ng/ml.

The Following Experimental Section Applies for the Preparation of the Compounds of Formula XI:

Abbreviations which are used in the descriptions of the schemes, preparations and the examples that follow are:
THF: Tetrahydrofuran
DMF: N,N-Dimethylformamide
EtOAc: Ethyl acetate
AcOH: Acetic acid
HOOBt: 3-Hydroxy-1,2,3-benzotriazin-4(3H)-one
EDCl: 1-(3-dimethylaminopropyl)-3-ethylcarbodiimide hydrochloride
NMM: N-Methylmorpholine
ADDP: 1,1′-(Azodicarbobyl)dipiperidine
DEAD: Diethylazodicarboxylate
MeOH: Methanol
EtOH: Ethanol
Et₂O: Diethyl ether
DMSO: Dimethylsulfoxide
HOBt: N-Hydroxybenzotriazole
PyBrOP: Bromo-tris-pyrrolidinophosphonium hexafluorophosphate
DCM: Dichloromethane
DCC: 1,3-Dicyclohexylcarbodiimide
TEMPO: 2,2,6,6-Tetramethyl-1-piperidinyloxy
Phg: Phenylglycine
Chg: Cyclohexylglycine
Bn: Benzyl
Bzl: Benzyl
Et: Ethyl
Ph: Phenyl
iBoc: isobutoxycarbonyl
iPr: isopropyl
^{t}Bu or Bu^{t}: tert-Butyl
Boc: tert-Butyloxycarbonyl
Cbz: Benzyloxycarbonyl
Cp: Cylcopentyldienyl
Ts: p-toluenesulfonyl
MCPBA: 3-chloroperbenzoic acid.
Me: Methyl
HATU: O-(7-azabenzotriazol-1-yl)-1,1,3,3-tetramethyluronium hexafluorophosphate
DMAP: 4-N,N-Dimethylaminopyridine
Bop: Benzotriazol-1-yl-oxy-tris(dimethylamino)hexafluorophosphate
PCC: Pyridiniumchlorochromate

Other abbreviations are commonly used abbreviations Such as according to the guidelines published by Journal of Organic Chemistry.

General Schemes for Preparation of Target Compounds

Compounds of the present invention were synthesized using the general schemes (Methods A-E) described below.

Method A

Deprotection of the N-Boc functionality of 1.01 under acidic conditions provided the hydrochloride salt 1.02 which was subsequently coupled with N-Boc-tert-leucine under peptide coupling methodology (Louis A Carpino et al. “Preparation of uronium and immonium salts for peptide coupling”, WO 2002094822, pp. 76) to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxyl amide 1.07. Oxidation (Moffatt, or Dess-Martin's) resulted in the target compound 1.08. 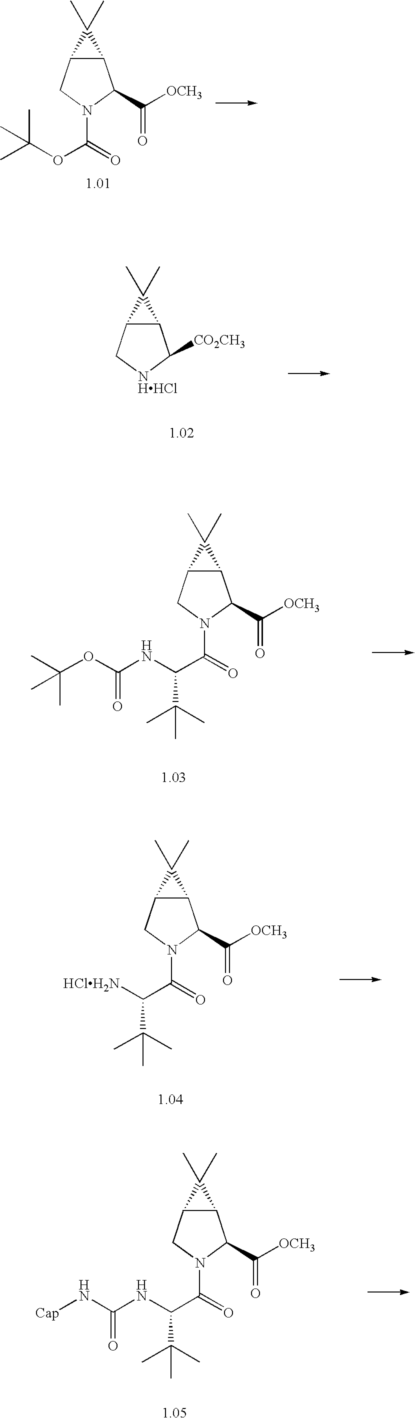 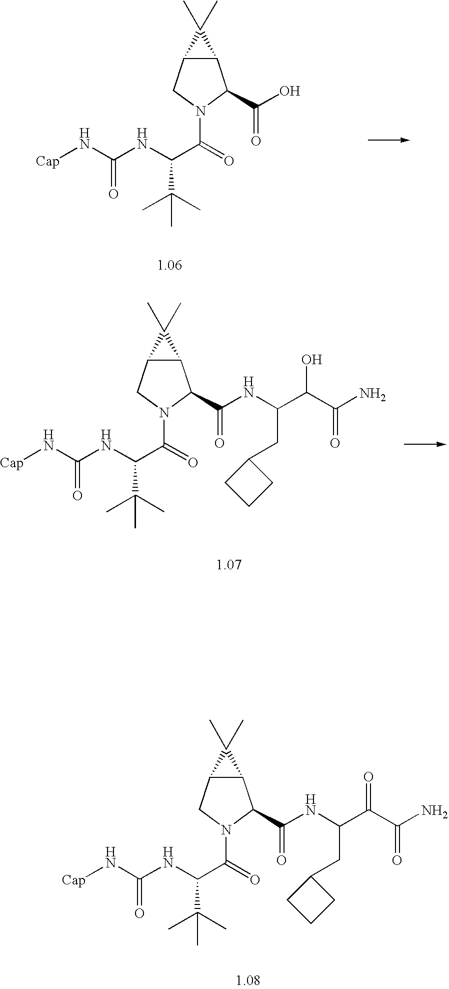
Method B

Peptide coupling of the acid 1.06 with the appropriate P₁-P′ secondary amide moiety afforded the hydroxyl amide 1.09. Oxidation (Moffatt or Dess-Martin's) resulted in the target compound 1.10.

Method C

In another variation, peptide coupling of the N-Boc-P2-P₃-acid 1.03 with the appropriate P₁-P′ amide moiety afforded the hydroxyl amide 1.11. Oxidation (Moffatt or Dess-Martin's) resulted in the keto-amide 1.12. Deprotection of the N-Boc using either formic acid or 4 M HCI in dioxane gave the formate or hydrochloride salt 1.13. Treatment with a suitable isocyanate (or isocyanate equivalent) resulted in the target compound 1.14. 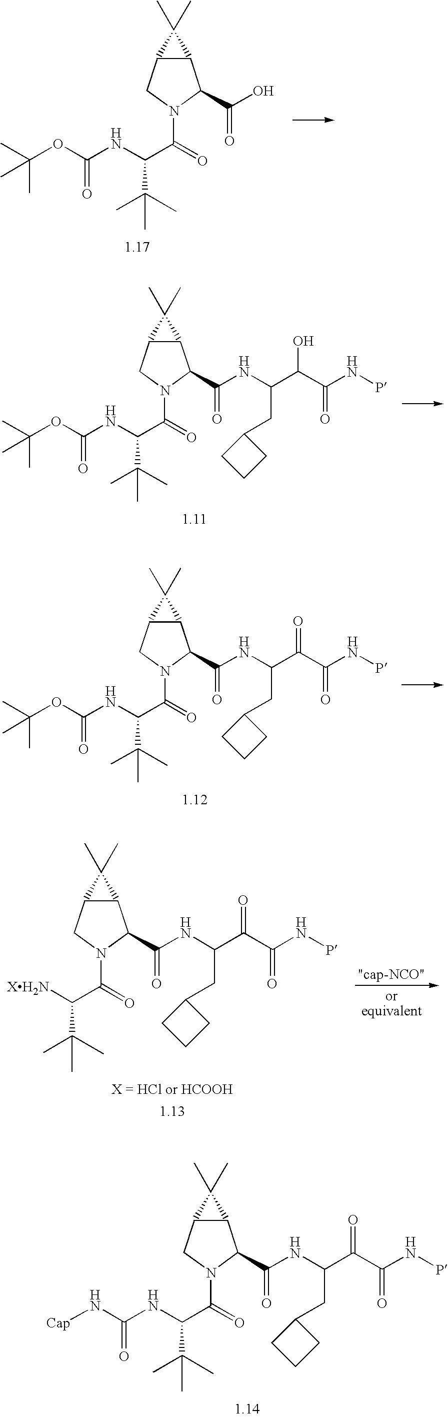
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 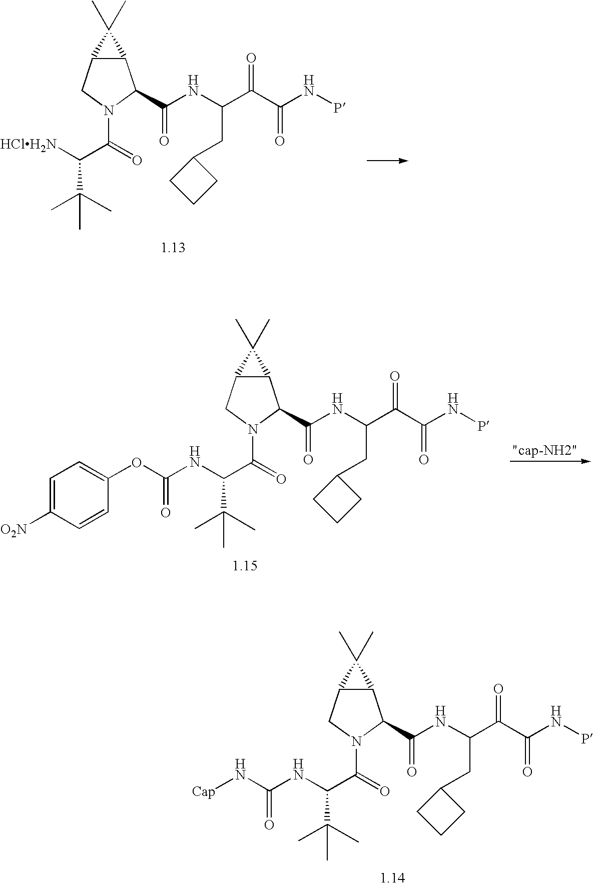
Method E

In yet another variation, the dipeptide hydrochloride salt 1.04 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14. 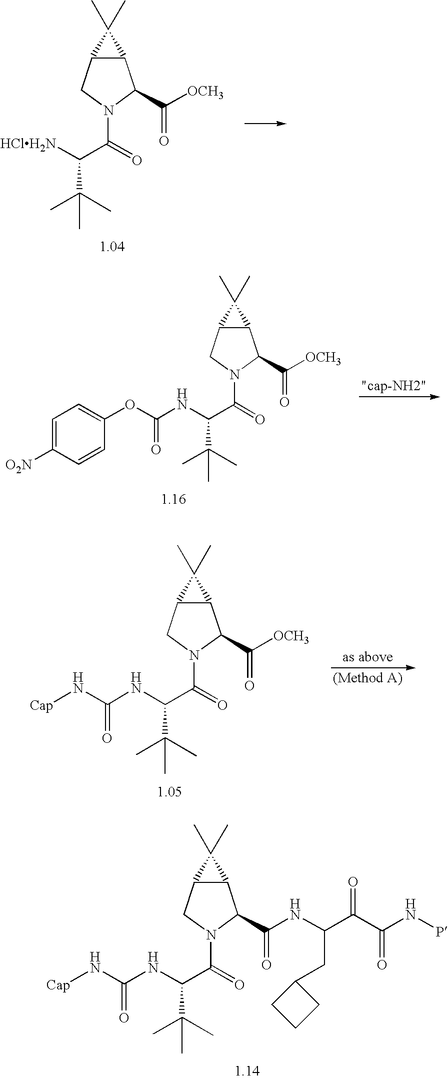
The Following Experimental Section Applies for the Preparation of the Compounds of Formula XII:

Abbreviations which are used in the descriptions of the schemes, preparations and the examples that follow are:
THF: Tetrahydrofuran
DMF: N,N-Dimethylformamide
EtOAc: Ethyl acetate
AcOH: Acetic acid
HOOBt: 3-Hydroxy-1,2,3-benzotriazin-4(3H)-one
EDCl: 1-(3-dimethylaminopropyl)-3-ethylcarbodiimide hydrochloride
NMM: N-Methylmorpholine
ADDP: 1,1′-(Azodicarbobyl)dipiperidine
DEAD: Diethylazodicarboxylate
MeOH: Methanol
EtOH: Ethanol
Et₂O: Diethyl ether
DMSO: Dimethylsulfoxide
HOBt: N-Hydroxybenzotriazole
PyBrOP: Bromo-tris-pyrrolidinophosphonium hexafluorophosphate
DCM: Dichloromethane
DCC: 1,3-Dicyclohexylcarbodiimide
TEMPO: 2,2,6,6-Tetramethyl-1-piperidinyloxy
Phg: Phenylglycine
Chg: Cyclohexylglycine
Bn: Benzyl
Bzl: Benzyl
Et: Ethyl
Ph: Phenyl
iBoc: isobutoxycarbonyl
iPr: isopropyl
^{t}Bu or Bu^{t}: tert-Butyl
Boc: tert-Butyloxycarbonyl
Cbz: Benzyloxycarbonyl
Cp: Cylcopentyldienyl
Ts: p-toluenesulfonyl
Me: Methyl
HATU: O-(7-azabenzotriazol-1-yl)-1,1,3,3-tetramethyluronium hexafluorophosphate
DMAP: 4-N,N-Dimethylaminopyridine
BOP: Benzotriazol-1-yl-oxy-tris(dimethylamino)hexafluorophosphate
PCC: Pyridiniumchlorochromate

### General Schemes for Preparation of Target Compounds

Compounds of the present invention were synthesized using the general schemes (Methods A-E) described below.

Method A:

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxyl amide 1.07. Oxidation (Moffatt or related process—T. T. Tidwell, Synthesis, 1990, 857; or Dess-Martin's—J. Org. Chem., 1983, 48, 4155) resulted in the target compound 1.08. 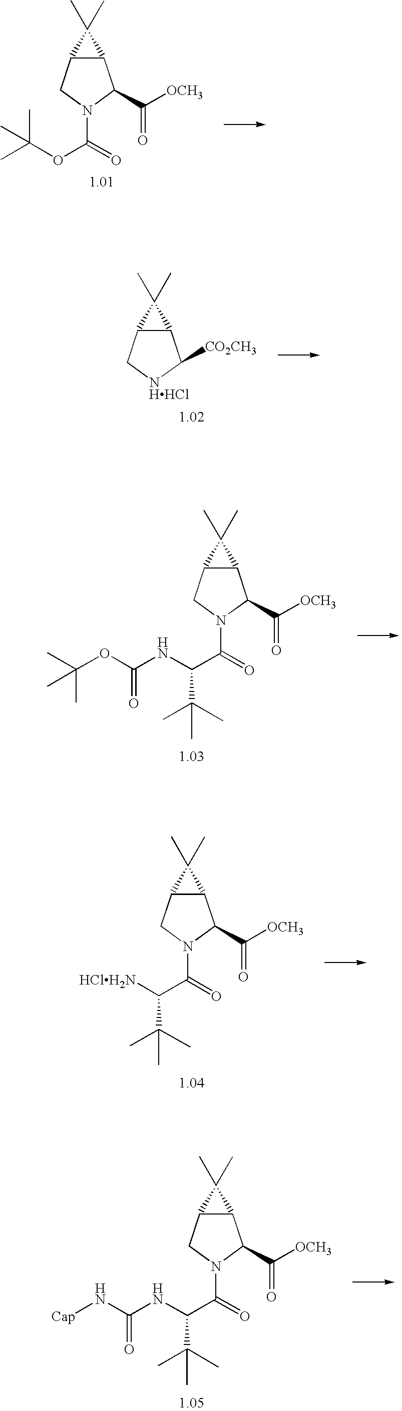 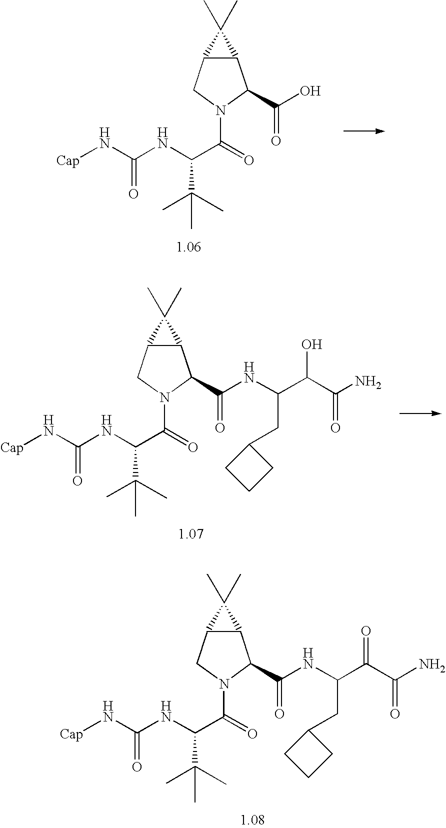
Method B

Peptide coupling of the acid 1.06 with the appropriate P₁-P′ secondary amide moiety afforded the hydroxyl amide 1.09. Oxidation (Moffatt or Dess-Martin's) resulted in the target compound 1.10. 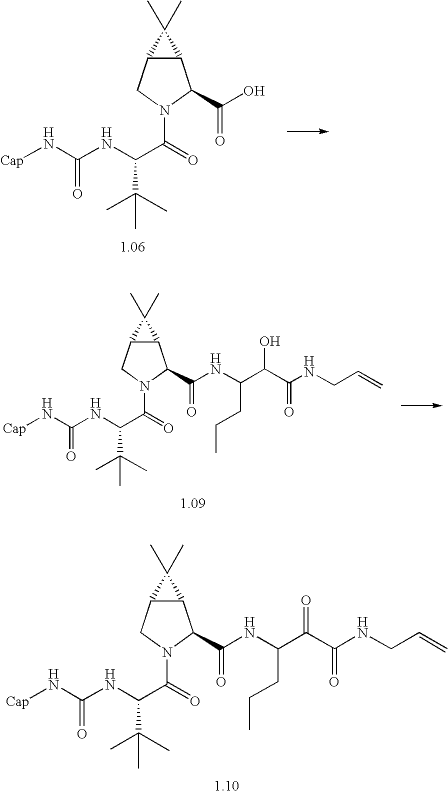
Method C

In another variation, peptide coupling of the N-Boc-P₂-P₃-acid 1.17 with the appropriate P₁-P′ amide moiety afforded the hydroxyl amide 1.11. Oxidation (Moffatt or Dess-Martin's) resulted in the keto amide 1.12. Deprotection of the N-Boc functionality gave the hydrochloride salt 1.13. Treatment with a suitable isocyanate (or isocyanate equivalent) resulted in the target compound 1.14. 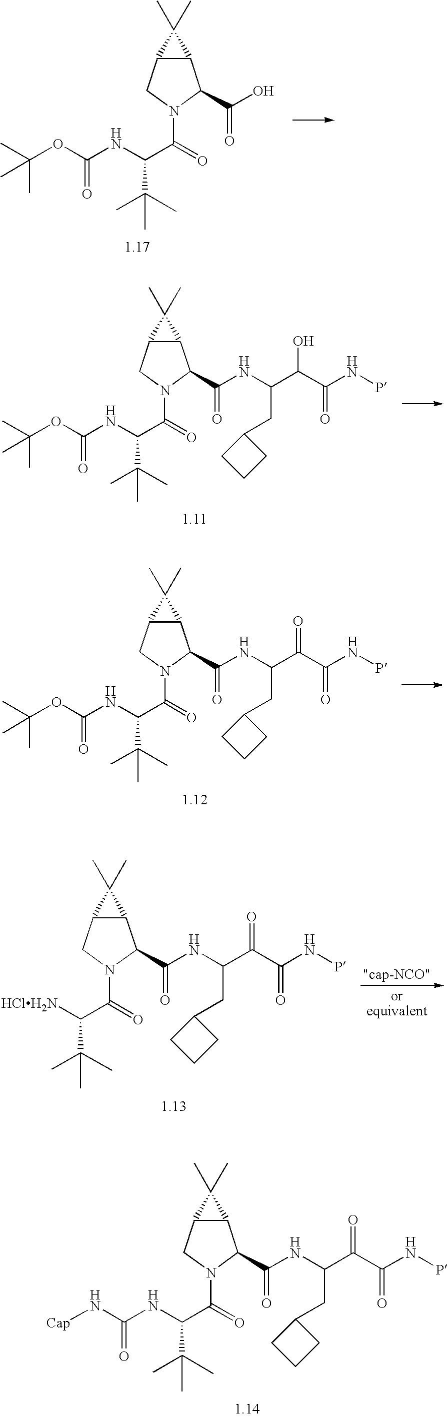
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 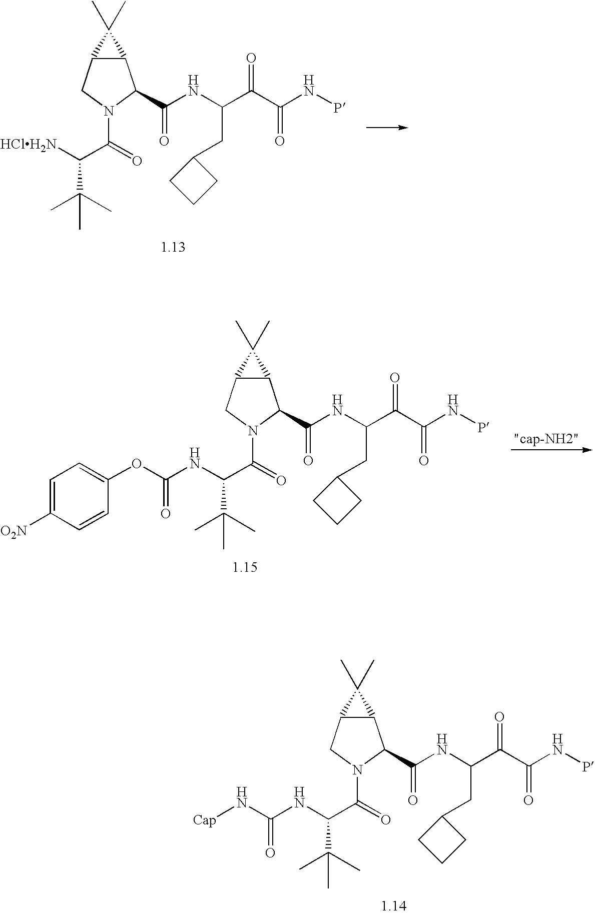
Method E

In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14.

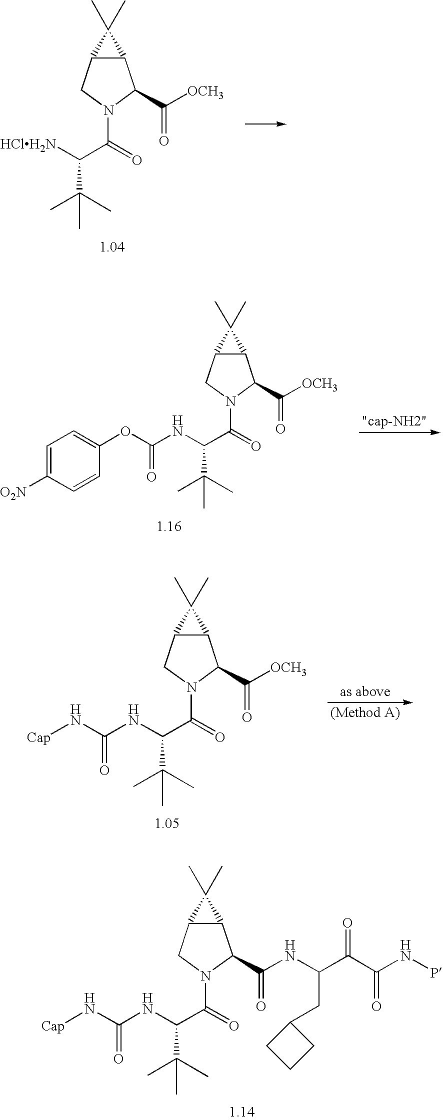

The following experimental section applies for the preparation of the compounds of Formula XIII:

Abbreviations which are used in the descriptions of the schemes, preparations and the examples that follow are:

THF: Tetrahydrofuran
DMF: N,N-Dimethylformamide
EtOAc: Ethyl acetate
AcOH: Acetic acid
HOOBt: 3-Hydroxy-1,2,3-benzotriazin-4(3H)-one
EDCl: 1-(3-dimethylaminopropyl)-3-ethylcarbodiimide hydrochloride
NMM: N-Methylmorpholine
ADDP: 1,1′-(Azodicarbobyl)dipiperidine
DEAD: Diethylazodicarboxylate
DIAD: Diisopropylazodicarboxylate
MeOH: Methanol
EtOH: Ethanol
Et₂O: Diethyl ether
DMSO: Dimethylsulfoxide
HOBt: N-Hydroxybenzotriazole
PyBrOP: Bromo-tris-pyrrolidinophosphonium hexafluorophosphate
DCM: Dichloromethane
DCC: 1,3-Dicyclohexylcarbodiimide
TEMPO: 2,2,6,6-Tetramethyl-1-piperidinyloxy
Phg: Phenylglycine
Chg: Cyclohexylglycine
Bn: Benzyl
Bz: Benzyl
Et: Ethyl
Ph: Phenyl
iBoc: isobutoxycarbonyl
iPr: isopropyl
^{t}Bu or Bu^{t}: tert-Butyl
Boc: tert-Butyloxycarbonyl
Cbz: Benzyloxycarbonyl
Cp: Cylcopentyldienyl
Ts: p-toluenesulfonyl
Me: Methyl
Ms or Mesyl: Methane sulfonyl
HATU: O-(7-azabenzotriazol-1-yl)-1,1,3,3-tetramethyluronium hexafluorophosphate
DMAP: 4-N,N-Dimethylaminopyridine
Bop: Benzotriazol-1-yl-oxy-tris(dimethylamino)hexafluorophosphate
PCC: Pyridiniumchlorochromate
DIBAL-H: diisopropyl aluminum hydride
rt or RT: Room temperature
quant.: Quantitative yield
h or hr: hour
min: minute
TFA: Trifluoroacetic acid

### General Schemes for Preparation of Target Compounds

Compounds of the present invention were synthesized using the general schemes (Methods A-E) described below.

Method A

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxyl amide 1.07. Oxidation (Moffatt or related process—T. T. Tidwell, Synthesis, 1990, 857; or Dess-Martin's periodinane (J. Org. Chem., 1983, 48, 4155) resulted in the target compound 1.08. 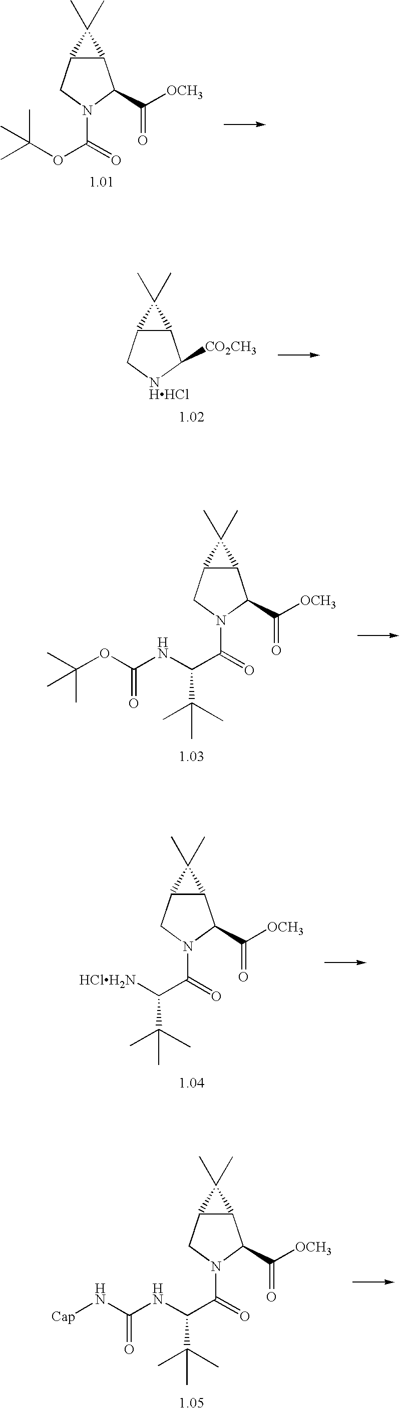 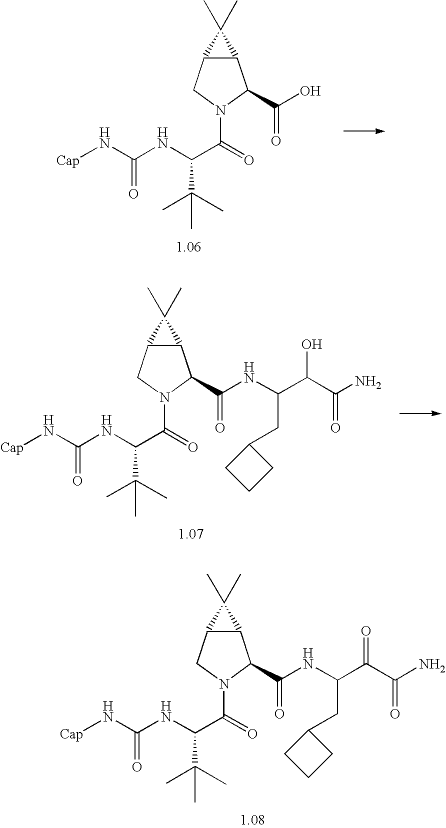
Method B

Peptide coupling of the acid 1.06 with the appropriate P₁-P′ secondary amide moiety afforded the hydroxyl amide 1.09. Oxidation (Moffatt or Dess-Martin's) resulted in the target compound 1.10. 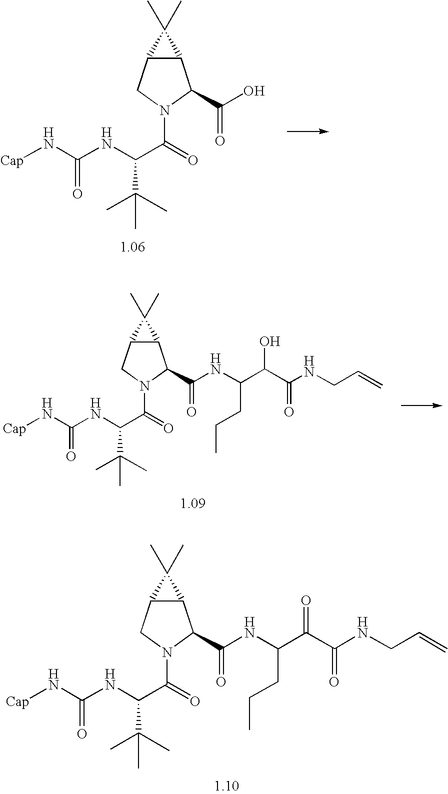
Method C

In another variation, peptide coupling of the N-Boc-P₂-P₃-acid 1.17 with the appropriate P₁-P′ amide moiety afforded the hydroxyl amide 1.11. Oxidation (Moffatt or Dess-Martin's) resulted in the keto amide 1.12. Deprotection of the N-Boc functionality gave the hydrochloride salt 1.13. Treatment with a suitable isocyanate (or isocyanate equivalent) resulted in the target compound 1.14. 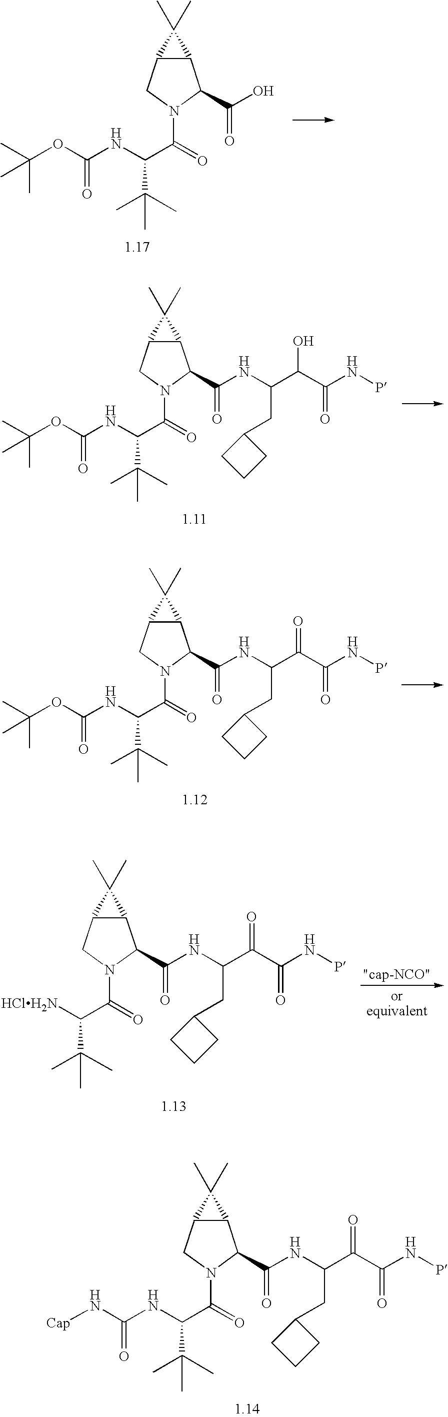
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 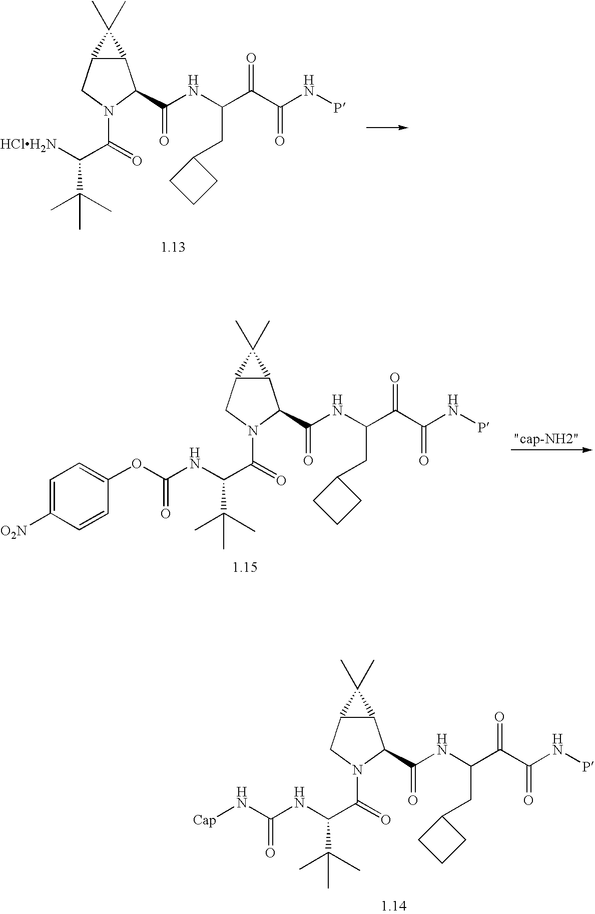
Method E

In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14. 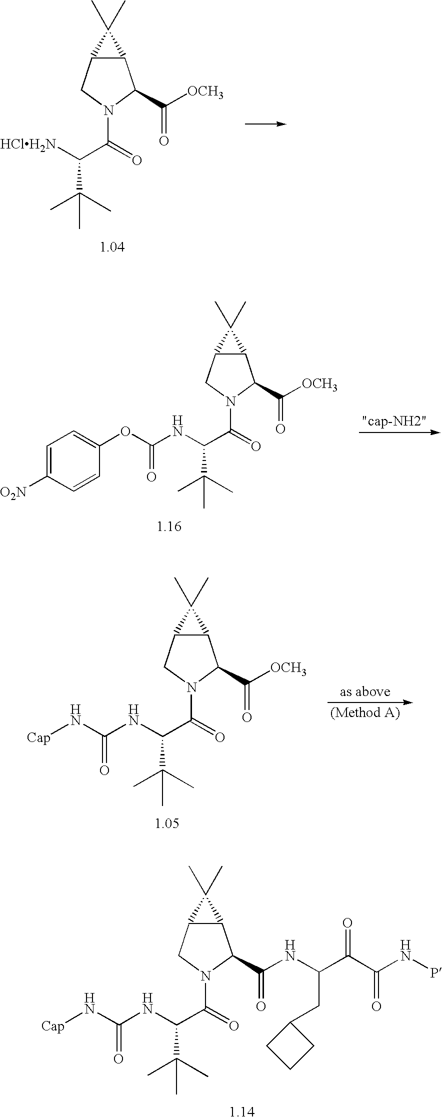
The Following Experimental Section Applies for the Preparation of the Compounds of Formula XIV:

For the procedures described below, the following abbreviations are used:
THF: Tetrahydrofuran
DMF: N,N-Dimethylformamide
EtOAc: Ethyl acetate
AcOH: Acetic acid
HOOBt: 3-Hydroxy-1,2,3-benzotriazin-4(3H)-one
EDCl: 1-(3-dimethylaminopropyl)-3-ethylcarbodiimide hydrochloride
NMM: N-Methylmorpholine
ADDP: 1,1′-(Azodicarbobyl)dipiperidine
DEAD: Diethylazodicarboxylate
MeOH: Methanol
EtOH: Ethanol
Et2O: Diethyl ether
DMSO: Dimethylsulfoxide
HOBt: N-Hydroxybenzotriazole
PyBrOP: Bromo-tris-pyrrolidinophosphonium hexafluorophosphate
DCM: Dichloromethane
DCC: 1,3-Dicyclohexylcarbodiimide
TEMPO: 2,2,6,6-Tetramethyl-1-piperidinyloxy
Phg: Phenylglycine
Chg: Cyclohexylglycine
Bn: Benzyl
Bzl: Benzyl
Et: Ethyl
Ph: Phenyl
DMF-DMA: N,N-Dimethylformamide-dimethylacetal
iBoc: isobutoxycarbonyl
iPr: isopropyl
^{t}Bu or Bu^{t}: tert-Butyl
Boc: tert-Butyloxycarbonyl
Cbz: Benzyloxycarbonyl
Cp: Cylcopentyldienyl
Ts: p-toluenesulfonyl
Me: Methyl
HATU: O-(7-azabenzotriazol-1-yl)-1,1,3,3-tetramethyluronium hexafluorophosphate
DMAP: 4-N,N-Dimethylaminopyridine
BOP: Benzotriazol-1-yl-oxy-tris(dimethylamino)hexafluorophosphate
PCC: Pyridiniumchlorochromate
KHMDS: Potassium Hexamethyldisilazide or Potassium bis(trimethylsilylamide)
NaHMDS: Sodium Hexamethyldisilazide or Sodium bis(trimethylsilylamide)
LiHMDS: Lithium Hexamethyldisilazide or Lithium bis(trimethylsilylamide)
10% Pd/C: 10% Palladium on carbon (by weight).
TG: Thioglycerol
   General Schemes for Preparation of Target Compounds

Compounds of the present invention were synthesized using the general schemes (Methods A-E) described below.

Method A

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxyl amide 1.07. Oxidation (Moffatt oxidation or related process—see, T. T. Tidwell, Synthesis, 1990, 857), or Dess-Martin Periodinane—J. Org. Chem., (1983) 48, 4155) resulted in the target compound 1.08. 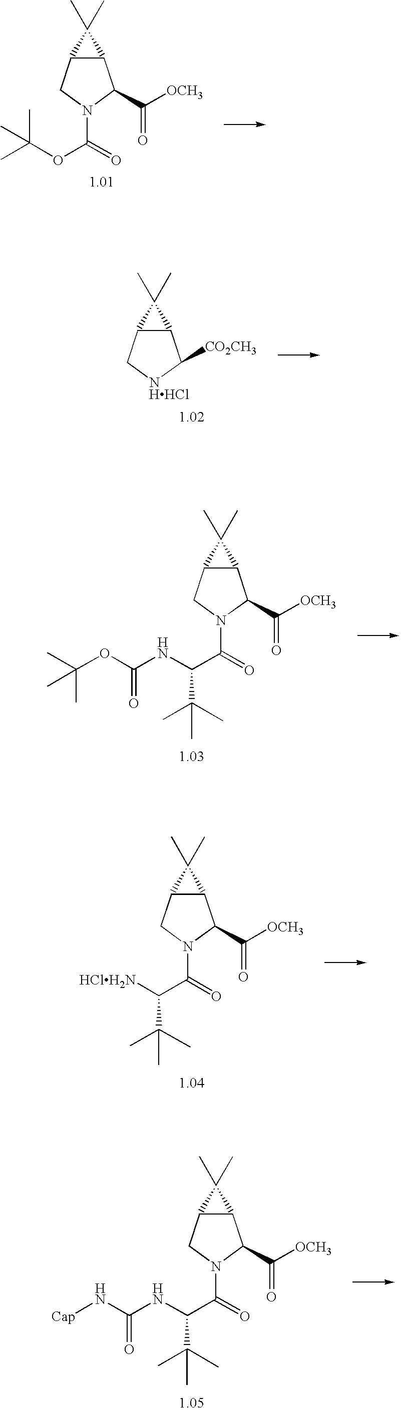 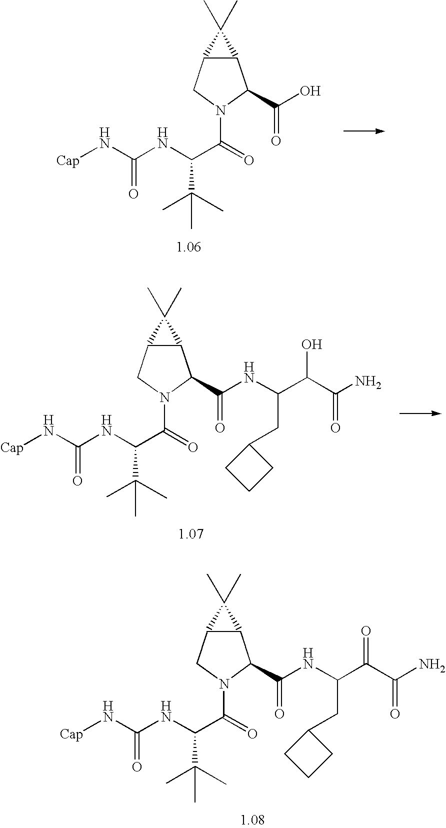
Method B

Peptide coupling of the acid 1.06 with the appropriate P₁-P′ secondary amide moiety afforded the hydroxyl amide 1.09. Oxidation (Moffatt or Dess-Martin's) resulted in the target compound 1.10. 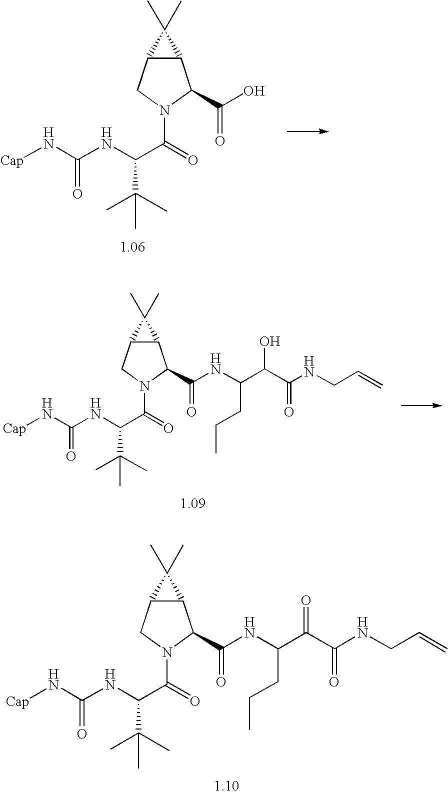
Method C

In another variation, peptide coupling of the N-Boc-P2-P₃-acid 1.17 with the appropriate P₁-P′ amide moiety afforded the hydroxyl amide 1.11. Oxidation (Moffatt or Dess-Martin Periodinane) resulted in the keto amide 1.12. Deprotection of the N-Boc functionality gave the hydrochloride salt 1.13. Treatment with a suitable isocyanate (or isocyanate equivalent) resulted in the target compound 1.14. 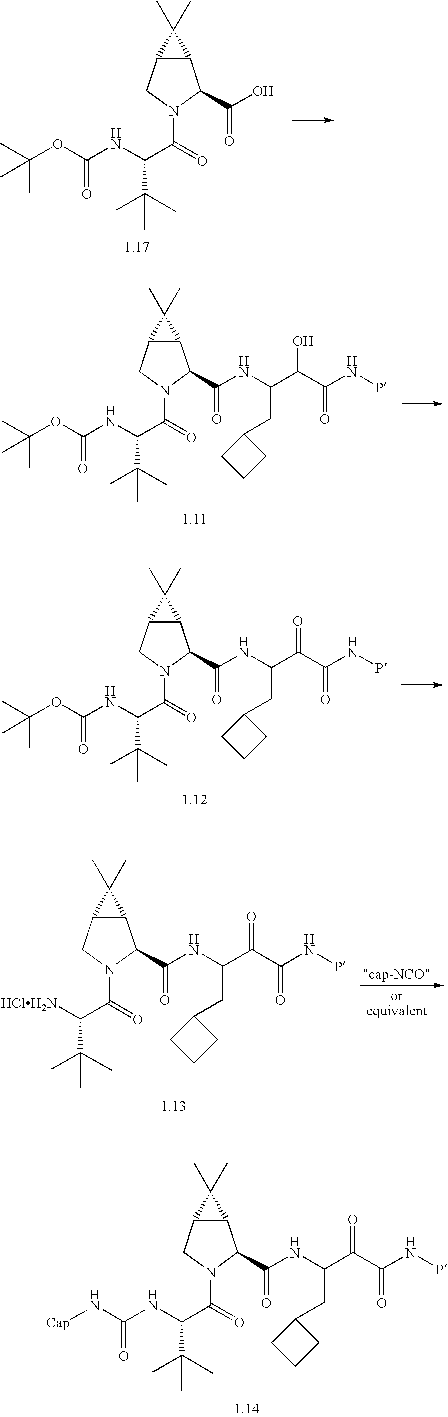
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 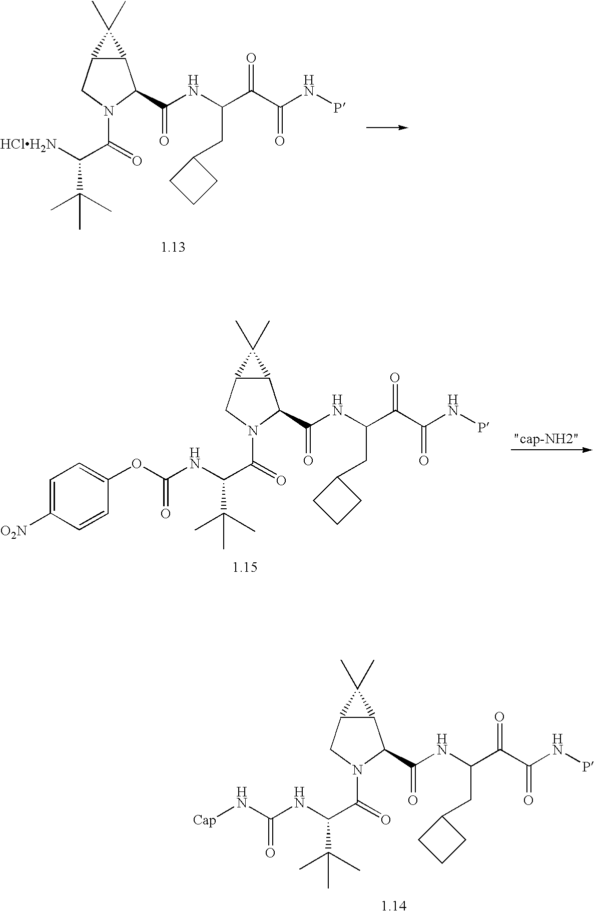
Method E

In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14. 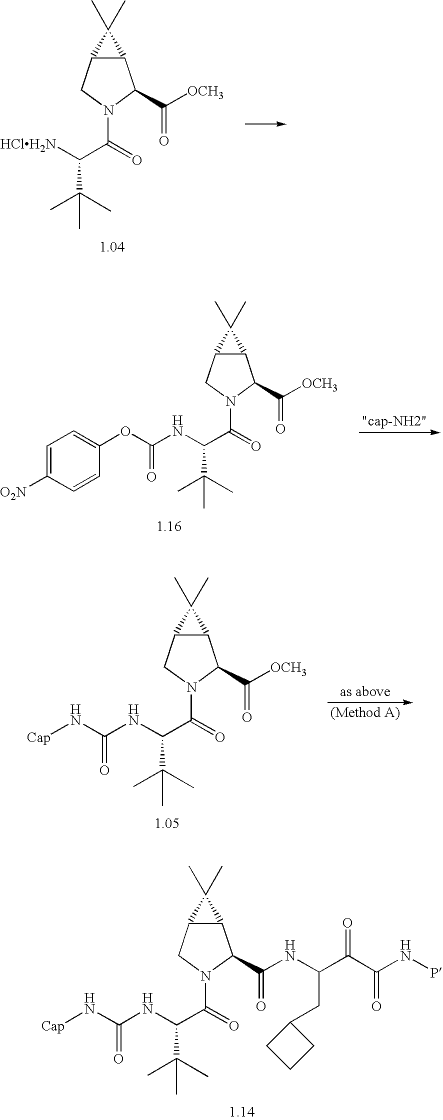
The Following Experimental Section Applies for the Preparation of the Compounds of Formula XV:

For the procedures described below, the following abbreviations are used:
THF: Tetrahydrofuran
DMF: N,N-Dimethylformamide
EtOAc: Ethyl acetate
AcOH: Acetic acid
HOOBt: 3-Hydroxy-1,2,3-benzotriazin-4(3H)-one
EDCl: 1-(3-dimethylaminopropyl)-3-ethylcarbodiimide hydrochloride
NMM: N-Methylmorpholine
ADDP: 1,1′-(Azodicarbobyl)dipiperidine
DEAD: Diethylazodicarboxylate
MeOH: Methanol
EtOH: Ethanol
Et2O: Diethyl ether
DMSO: Dimethylsulfoxide
HOBt: N-Hydroxybenzotriazole
PyBrOP: Bromo-tris-pyrrolidinophosphonium hexafluorophosphate
DCM: Dichloromethane
DCC: 1,3-Dicyclohexylcarbodiimide
TEMPO: 2,2,6,6-Tetramethyl-1-piperidinyloxy
Phg: Phenylglycine
Chg: Cyclohexylglycine
Bn: Benzyl
Bzl: Benzyl
Et: Ethyl
Ph: Phenyl
iBoc: isobutoxycarbonyl
iPr: isopropyl
^{t}Bu or Bu^{t}: tert-Butyl
Boc: tert-Butyloxycarbonyl
Cbz: Benzyloxycarbonyl
Cp: Cylcopentyldienyl
Ts: p-toluenesulfonyl
Me: Methyl
HATU: O-(7-azabenzotriazol-1-yl)-1,1,3,3-tetramethyluronium hexafluorophosphate
DMAP: 4-N,N-Dimethylaminopyridine
BOP: Benzotriazol-1-yl-oxy-tris(dimethylamino)hexafluorophosphate
PCC: Pyridiniumchlorochromate
KHMDS: Potassium Hexamethyldisilazide or Potassium bis(trimethylsilylamide)
NaHMDS: Sodium Hexamethyidisilazide or Sodium bis(trimethylsilylamide)
LiHMDS: Lithium Hexamethyldisilazide or Lithium bis(trimethylsilylamide)
10% Pd/C: 10% Palladium on carbon (by weight).

### Preparative Example 1

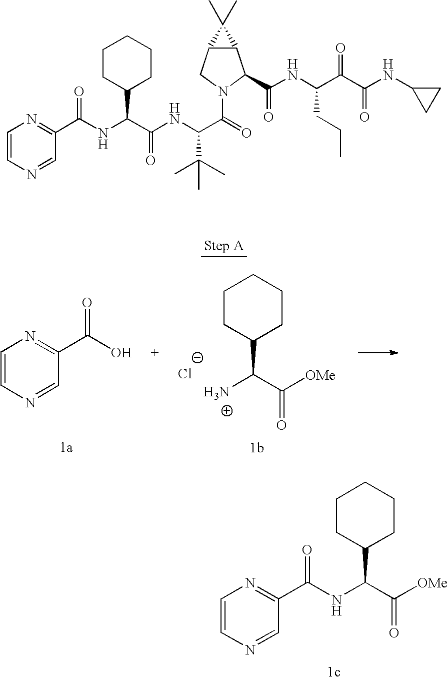

A solution of pyrazinecarboxylic acid 1a (3 g) in 150 mL of dry dichloromethane and 150 mL of dry DMF was stirred at 0° C. and treated with HATU (1.4 eq, 6.03 g). L-cyclohexylglycine hydrochloride 1b (1.2 eq, 6.03 g) was added in small portions. Then, N-methylmorpholine (4 eq, 10 mL, d 0.920) was added dropwise. The reaction mixture was gradually warmed to room temperature and stirred for 20 h. All the volatiles were removed under vacuum and the residue was dissolved in 500 mL of ethyl acetate. The organic layer was washed with water (100 mL), aqueous 1N HCl (100 mL), aqueous saturated sodium bicarbonate solution (100 mL), and brine (100 mL). The organic layer was dried over magnesium sulfate, filtered and concentrated under reduced pressure. The residue was chromatographed on silica gel (gradient: acetone/hexanes; 5:95 to 3:7) to afford the product 1c as a white solid.

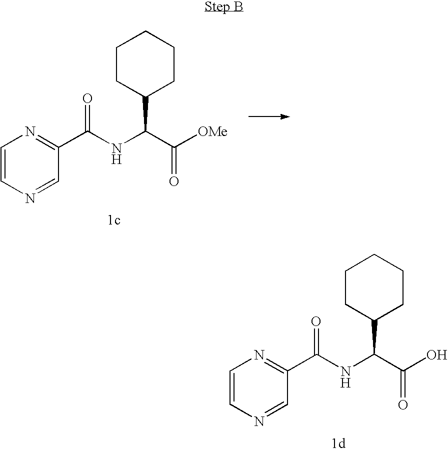

A solution of methyl ester 1c (6.5 g) in 270 mL of a 1:1:1 mixture of THF/MeOH/water was cooled to 0° C. and treated with lithium hydroxide monohydrate (2.5 eq, 2.45 g). The mixture was stirred and monitored by TLC (acetone/hexanes; 2:8). When all the starting material had been consumed, the reaction mixture was treated with 100 mL of aqueous 1N HCl and the mixture was concentrated on the rotavap. Dichloromethane (250 mL) was added and layers separated. The aqueous layer was extracted with dichloromethane (3×80 mL). The combined organic layers were dried over magnesium sulfate, filtered, and concentrated to afford the product 1d as a white solid.

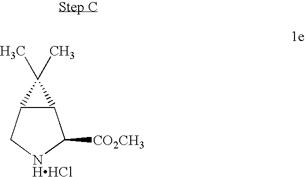

The amino ester 1e was prepared following the method of R. Zhang and J. S. Madalengoitia (J. Org. Chem. 1999, 64, 330), with the exception that the Boc group was cleaved by the reaction of the Boc-protected amino acid with methanolic HCl (4M HCl in dioxane was also employed for the deprotection).
(Note: In a variation of the reported synthesis, the sulfonium ylide was replaced with the corresponding phosphonium ylide).

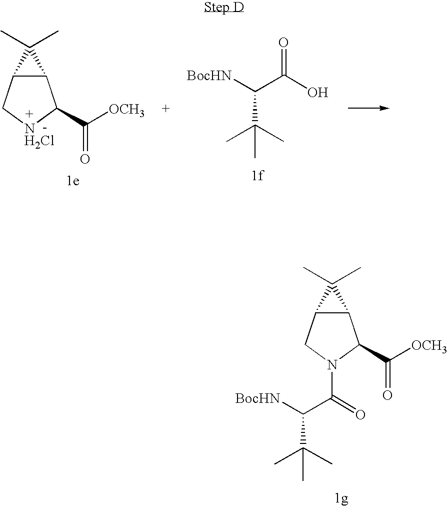

A solution of Boc-tert-Leu 1f (Fluka, 5.0 g, 21.6 mmol) in dry CH₂Cl₂/DMF (50 mL, 1:1) was cooled to 0° C. and treated with the amine hydrochloride le (5.3 g, 25.7 mmol), NMM (6.5 g, 64.8 mmol) and BOP reagent (11.6 g, 25.7 mmol). The reaction was stirred at rt. for 24h, diluted with aqueous HCl (1 M) and extracted with CH₂Cl₂. The combined organic layers were washed with aqueous 1 M HCl, saturated NaHCO₃, brine, dried (MgSO₄), filtered and concentrated in vacuo and purified by chromatography (SiO₂, Acetone/Hexane 1:5) to yield 1g as a colorless solid.

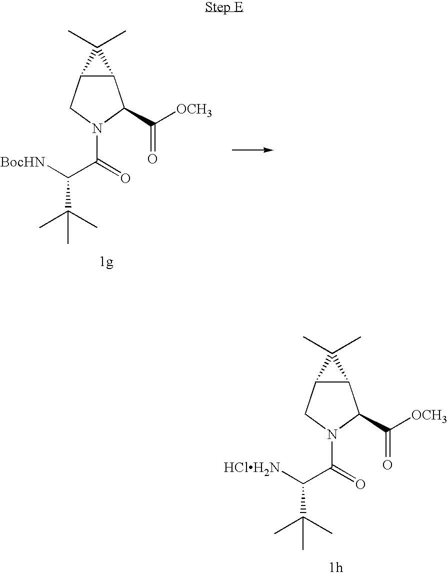

A solution of methyl ester 1g (4.0 g, 10.46 mmol) was dissolved in 4M HCl in dioxane and stirred at rt. for 3 h. The reaction mixture was concentrated in vacuo to obtain the amine hydrochloride salt, 1h which was used without purification.

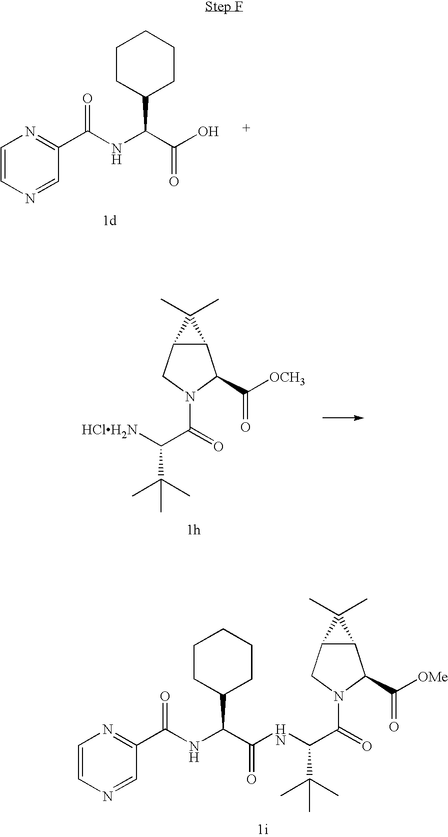

A solution of acid 1d (100 mg) in 5 mL of dry dichloromethane and 5 mL of dry DMF was stirred at 0° C. and treated with HATU (1.4 eq, 202 mg). The amine hydrochloride 1h (1.2 eq, 146 mg) was added. Then, N-methylmorpholine (4 eq, 0.17 mL, d 0.920) was also added. The reaction mixture was stirred at 0° C. overnight. All the volatiles were removed under vacuum and the residue was dissolved in 80 mL of ethyl acetate. The organic layer was washed with water (10 mL), aqueous 1N HCl (10 mL), aqueous saturated sodium bicarbonate solution (10 mL), and brine (10 mL). The organic layer was dried over magnesium sulfate, filtered and concentrated under reduced pressure. The residue was chromatographed on silica gel (gradient: acetone/hexanes; 1:9 to 4:6) to afford the product 1i as a white solid.

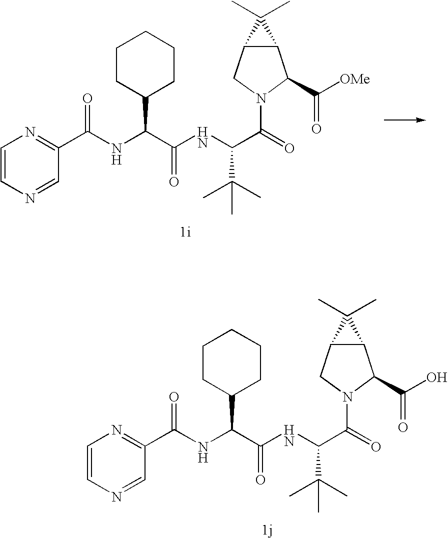

A solution of methyl ester 1i (180 mg) in 9 mL of a 1:1:1 mixture of THF/MeOH/water was cooled to 0° C. and treated with lithium hydroxide monohydrate (2.5 eq, 35 mg). The mixture was stirred and monitored by TLC (acetone/hexanes; 3:7). When all the starting material had been consumed, the reaction mixture was treated with 50 mL of aqueous 1 N HCl and the mixture was concentrated on the rotavap. Dichloromethane (80 mL) was added and layers separated. The aqueous layer was extracted with dichloromethane (3×50 mL). The combined organic layers were dried over magnesium sulfate, filtered, and concentrated to afford the product 1j as a white solid.

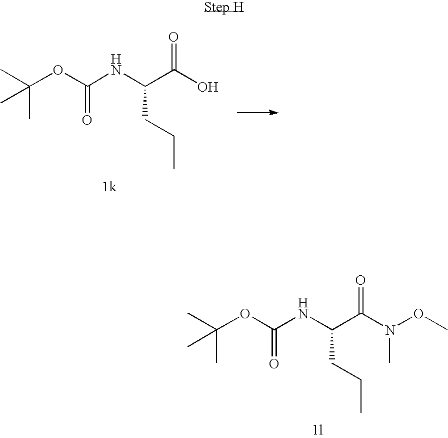

A solution of acid 1k (2 g) in 100 mL of dry dichloromethane and 5 mL of DMF was treated with N,O-dimethylhydroxylamine hydrochloride (1.1 eq, 986 mg), BOP reagent (1.1 eq, 4.47 g), and N-methylmorpholine (3.3 eq, 3.3 mL, d 0.920) in that order. The mixture was heated to 50° C. overnight. The reaction mixture was concentrated to half its volume and diluted with 400 mL of ethyl acetate. The organic layer was washed with water (80 mL), aqueous 1M HCl (80 mL), aqueous saturated sodium bicarbonate solution (80 mL), and brine (80 mL). The organic layer was dried over magnesium sulfate, filtered, and concentrated under reduced pressure. The residue was chromatographed on silica gel (gradient: acetone/hexanes; 5:95 to 3:7) to afford the product 1l as a clear oil.

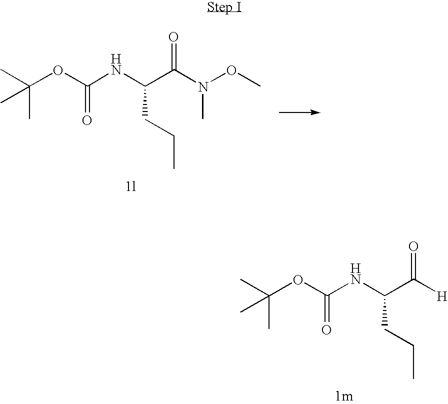

A solution of amide 1l (2.2 g) in 100 mL of dry THF was cooled to ° C. Lithium aluminum hydride solution (1.3 eq) was added dropwise. The cooling bath was removed after 5 min and the mixture was allowed to reach room temperature. TLC analysis (ethyl acetate/hexanes; 2:8) showed that all the starting material had been consumed. The excess LAH was carefully quenched by addition of drops of aqueous saturated sodium hydrogen sulfate. The mixture was diluted with 200 mL of ether and aqueous saturated sodium hydrogen sulfate was added in small portions until a white solid precipitated. The mixture was filtered thru celite and the filtrate was washed with 50 mL of brine. The organic layer was dried over magnesium sulfate, filtered and concentrated. The residue was chromatographed on silica gel (gradient: ethyl acetate/hexanes; 5:95 to 4:6) to afford the aldehyde product 1m as a colorless oil.

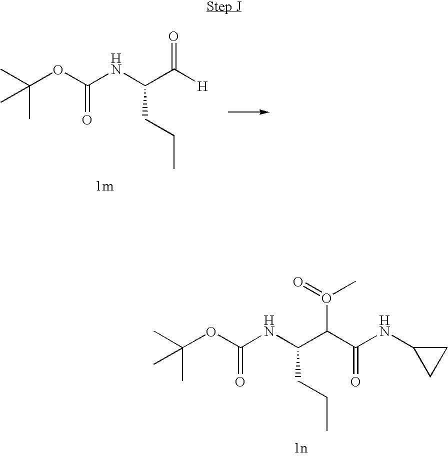

A solution of aldehyde 1m (1.8 g) in 100 mL of dry dichloromethane was treated with isonitrile (1.1 eq, 680 mg) and acetic acid (2 eq, 1.02 mL, d 1.0149). The mixture was stirred overnight. All the volatiles were removed under vacuum and the residue was chromatographed on silica gel (gradient: ethyl acetate/hexanes; 2:8 to 6:4) to afford the product 1n as a white solid.

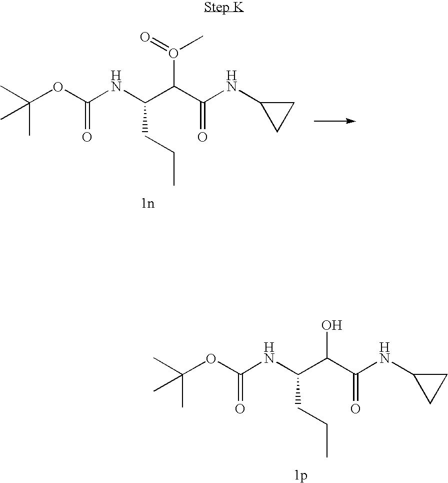

A solution of acetate 1n (1.6 g) in 60 mL of a 1:1:1 mixture of THF/MeOH/water was treated with lithium hydroxide monohydrate and stirred for approximately 1 h until all the starting material had been consumed as determined by TLC analysis (ethyl acetate/hexanes; 1:1). The volatiles were removed in rotavap and the residue was diluted with dichloromethane (150 mL). The layers were separated and the aqueous layer was diluted with 30 mL of aqueous saturated sodium bicarbonate solution and extracted with dichloromethane (3×80 mL). The combined organic layers were dried over magnesium sulfate, filtered and concentrated to afford the product 1p as a white solid.

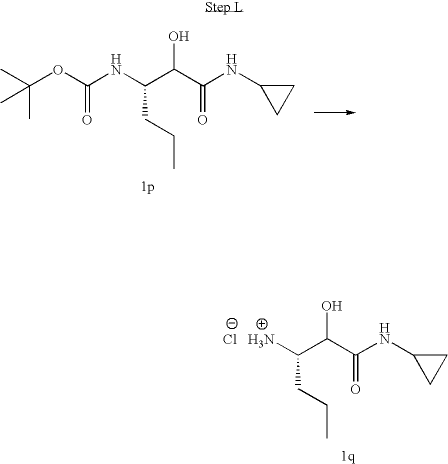

The N-Boc protected amine 1p (1.5 g) was dissolved in 20 mL of 4M HCl in dioxane. The reaction mixture was stirred for about 1 h until all the starting material had been consumed. All the volatiles were removed under vacuum to afford the product 1q as a white solid.

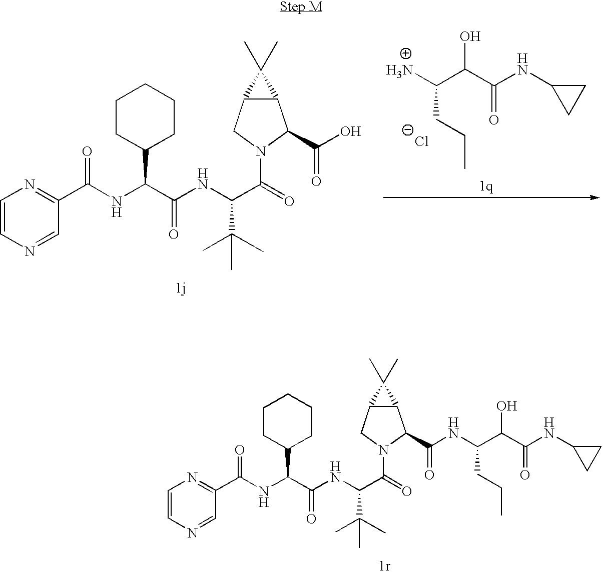

A solution of acid 1j (50 mg) in 2 mL of dry dichloromethane and 2 mL of dry DMF was stirred at 0° C. and treated with HATU (1.4 eq, 52 mg). The amine hydrochloride 1q (1.2 eq, 26 mg) was added. Then, N-methylmorpholine (4 eq, 0.042 mL, d 0.920) was also added. The reaction mixture was stirred at 0° C. overnight. All the volatiles were removed under vacuum and the residue was dissolved in 80 mL of ethyl acetate. The organic layer was washed with water (10 mL), aqueous 1N HCl (10 mL), aqueous saturated sodium bicarbonate solution (10 mL), and brine (10 mL). The organic layer was dried over magnesium sulfate, filtered and concentrated under reduced pressure. The product 1r was used without further purification.

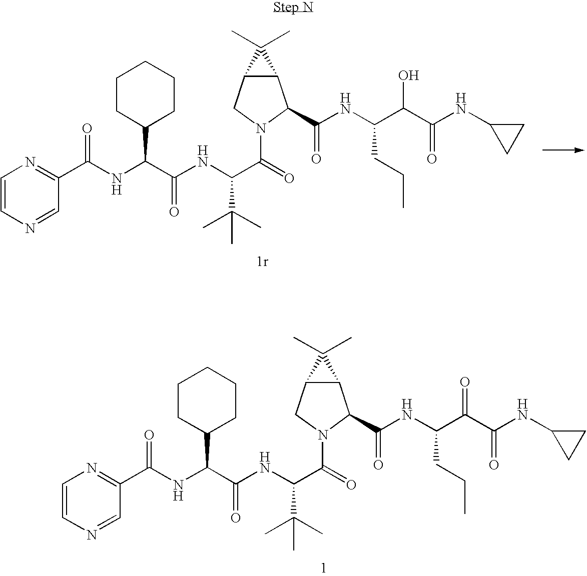

A solution of alcohol 1r (65 mg) in 5 mL of dry dichloromethane was treated with Dess-Martin periodinane (3 eq, 121 mg). Reaction mixture was stirred at room temperature for 45 min. The mixture was treated with aqueous 1M sodium thiosulfate solution (10 mL) and aqueous saturated sodium bicarbonate solution (10 mL) and stirred for 15 min. The mixture was extracted with dichloromethane (3×20 mL). The combined organic layers were dried over magnesium sulfate, filtered, and concentrated. The residue was chromatographed on silica gel (gradient: acetone/hexanes; 2:8 to 5:5) to afford the product 1 as a white solid.

One skilled in the art would understand that other suitable compounds of Formula XV can be prepared in a similar manner to that disclosed above.

The Following Experimental Section Applies for the Preparation of the Compounds of Formula XVI:

### Preparative Example A

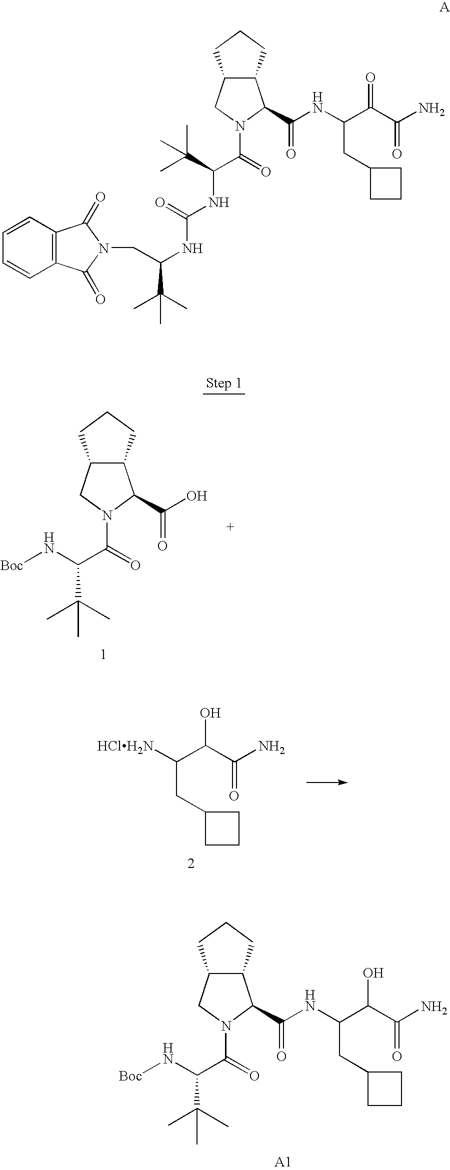

A solution of acid 1 (255 mg) in 5 mL of dry dichloromethane and 5 mL of dry DMF was stirred at 0° C. and treated with HATU (368 mg). The amine hydrochloride 2 (201 mg) was added followed by addition of N-methylmorpholine (0.42 mL). The reaction mixture was gradually warmed to room temperature and stirred overnight. All the volatiles were removed under vacuum and the residue was taken into 100 mL of ethyl acetate. The organic layer was washed with aqueous 1N HCl (15 mL), aqueous saturated NaHCO3 (15 mL), water (15 mL), brine (15 mL), dried over MgSO4, filtered, and concentrated under reduced pressure to afford the desired product A1. No further purification was carried out for the product.

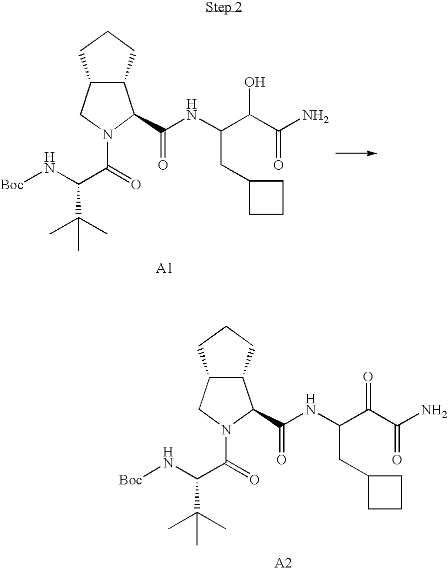

A solution of A1 (360 mg) in 20 mL of a 1:1 mixture of toluene/DMSO was treated with EDCl (1.3 g) and dichloroacetic acid (0.42 mL, d 1.563). Reaction mixture was stirred at room temperature for about 3 h. The reaction mixture was diluted with dichloromethane (100 mL) and washed with aqueous saturated NaHCO₃ (15 mL), aqueous 1N HCl (15 mL), and brine (15 mL). The organic layer was dried over magnesium sulfate, filtrated, and concentrated under reduced pressure. The residue was chromatographed on silica gel (gradient: acetone/hexanes; 2:8 to 5:5) to afford the product A2 in 84% yield.

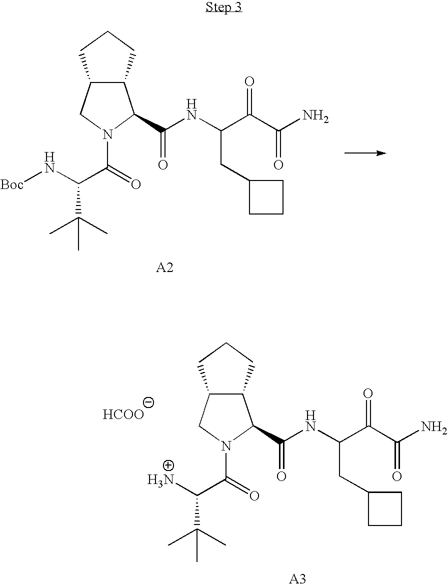

The N-Boc protected amine A2 was treated with 10 mL of formic acid. The resulting solution was stirred for 2 h. All the volatiles were removed under reduced pressure. No further purification was done for the product A3.

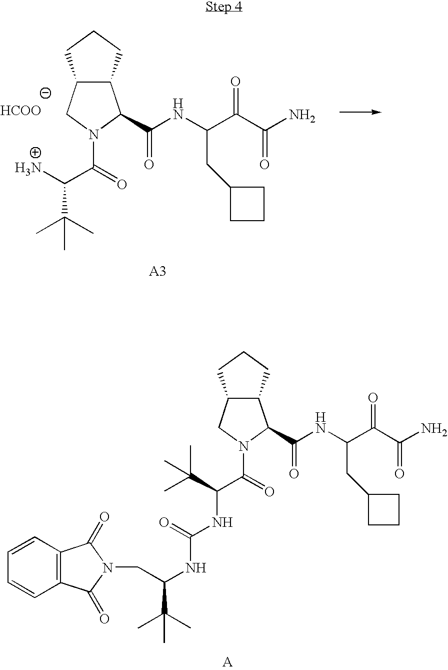

To a solution of the amine salt A3 in 1 mL of dry methylene chloride was added N-methylmorpholine (0.037 mL, d 0.920). The resulting solution was cooled in an ice-water bath and a solution of isocyanate in toluene (2.5 mL of a 0.135M soln) was slowly added. The mixture was stirred for 2 h (temp 0 to 25° C.). The reaction mixture was diluted with 60 mL of dichloromethane and washed with 15 mL of aqueous 1N HCl. Aqueous layer was back extracted with dichloromethane (2×20 mL). Combined organic layers were dried over magnesium sulfate, filtered and concentrated under reduced pressure. The residue was chromatographed on Silica gel (gradient: acetone/hexanes; 1:9 to 6:4) to give the product A (15 mg) as a white solid in 20% yield. HRMS (FAB) calcd for C₃₇H₅₃N₆O₇ [M+H] 693.3976. found 693.3987.

One skilled in the art would understand that other suitable compounds of Formula XVI can be prepared in a similar manner to that disclosed above.

The Following Experimental Section Applies for the Preparation of the Compounds of Formula XVII:

Abbreviations which are used in the descriptions of the schemes, preparations and the examples that follow are:
THF: Tetrahydrofuran
DMF: N,N-Dimethylformamide
EtOAc: Ethyl acetate
AcOH: Acetic acid
HOOBt: 3-Hydroxy-1,2,3-benzotriazin-4(3H)-one
EDCl: 1-(3-dimethylaminopropyl)-3-ethylcarbodiimide hydrochloride
NMM: N-Methylmorpholine
ADDP: 1,1′-(Azodicarbobyl)dipiperidine
DEAD: Diethylazodicarboxylate
MeOH: Methanol
EtOH: Ethanol
Et2O: Diethyl ether
DMSO: Dimethylsulfoxide
HOBt: N-Hydroxybenzotriazole
PyBrOP: Bromo-tris-pyrrolidinophosphonium hexafluorophosphate
DCM: Dichloromethane
DCC: 1,3-Dicyclohexylcarbodiimide
TEMPO: 2,2,6,6-Tetramethyl-1-piperidinyloxy
Phg: Phenylglycine
Chg: Cyclohexylglycine
Bn: Benzyl
Bzl: Benzyl
Et: Ethyl
Ph: Phenyl
iBoc: isobutoxycarbonyl
iPr: isopropyl
^{t}Bu or Bu^{t}: tert-Butyl
Boc: tert-Butyloxycarbonyl
Cbz: Benzyloxycarbonyl
Cp: Cylcopentyldienyl
Ts: p-toluenesulfonyl
Me: Methyl
HATU: O-(7-azabenzotriazol-1-yl)-1,1,3,3-tetramethyluronium hexafluorophosphate
DMAP: 4-N,N-Dimethylaminopyridine
BOP : Benzotriazol-1-yl-oxy-tris(dimethylamino)hexafluorophosphate
PCC: Pyridiniumchlorochromate
KHMDS: Potassium Hexamethyldisilazide or Potassium bis(trimethylsilylamide)
NaHMDS: Sodium Hexamethyldisilazide or Sodium bis(trimethylsilylamide)
LiHMDS: Lithium Hexamethyldisilazide or Lithium bis(trimethylsilylamide)
10% Pd/C: 10% Palladium on carbon (by weight).
TG: Thioglycerol
   General Schemes for Preparation of Target Compounds

Compounds of the present invention were synthesized using the general schemes (Methods A-E) described below.

Method A

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxyl amide 1.07. Oxidation (Moffatt oxidation or related process—see, T. T. Tidwell, Synthesis, 1990, 857), or Dess-Martin Periodinane—J. Org. Chem., (1983) 48, 4155) resulted in the target compound 1.08. 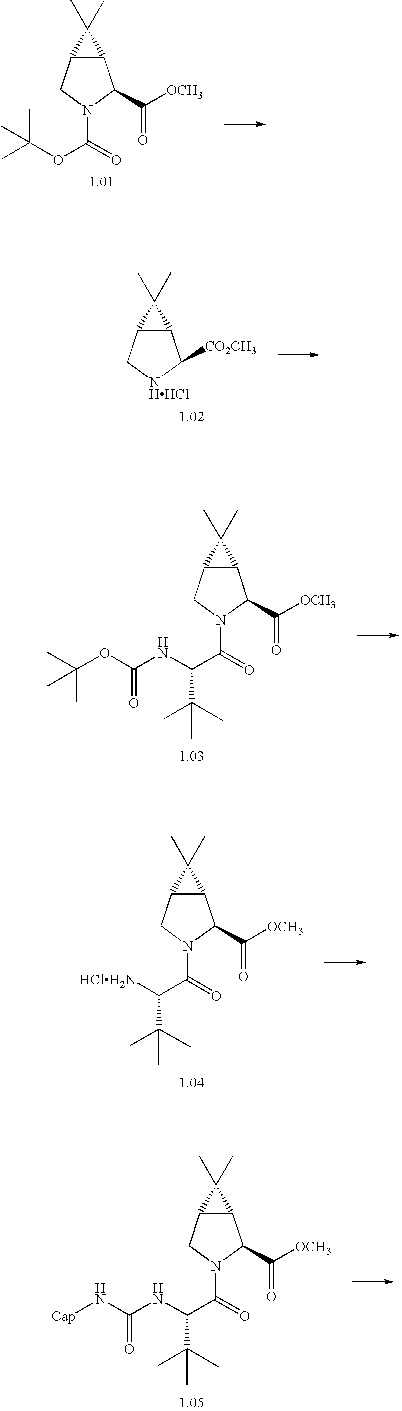 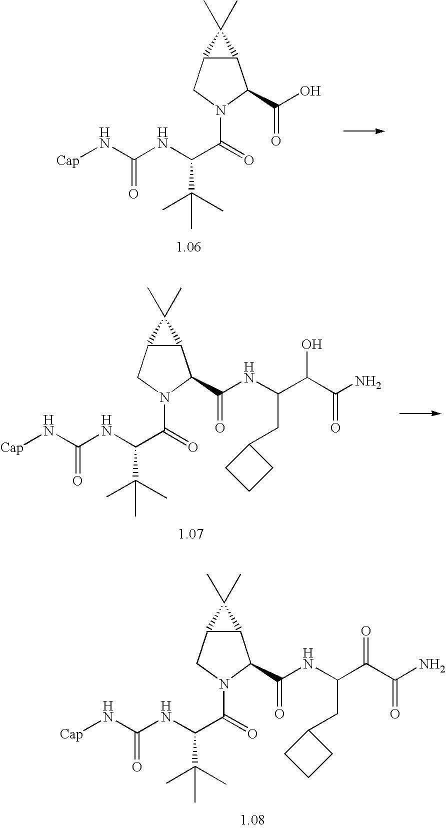
Method B

Peptide coupling of the acid 1.06 with the appropriate P₁-P′ secondary amide moiety afforded the hydroxyl amide 1.09. Oxidation (Moffatt or Dess-Martin's) resulted in the target compound 1.10. 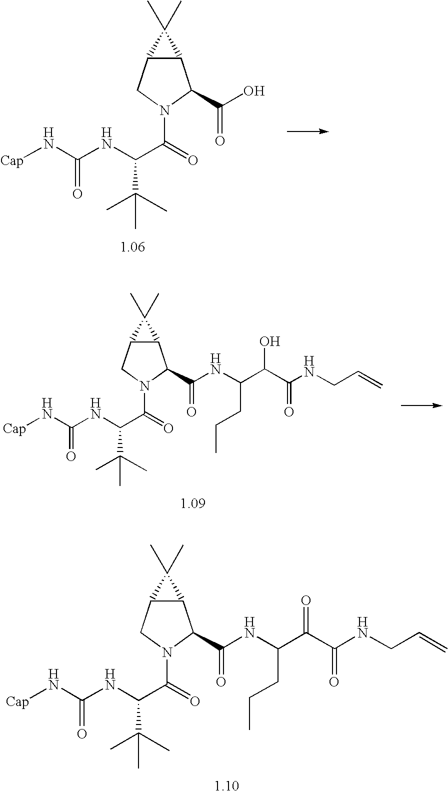
Method C

In another variation, peptide coupling of the N-Boc-P2-P₃-acid 1.17 with the appropriate P₁-P′ amide moiety afforded the hydroxyl amide 1.11. Oxidation (Moffatt or Dess-Martin Periodinane) resulted in the keto amide 1.12. Deprotection of the N-Boc functionality gave the hydrochloride salt 1.13. Treatment with a suitable isocyanate (or isocyanate equivalent) resulted in the target compound 1.14. 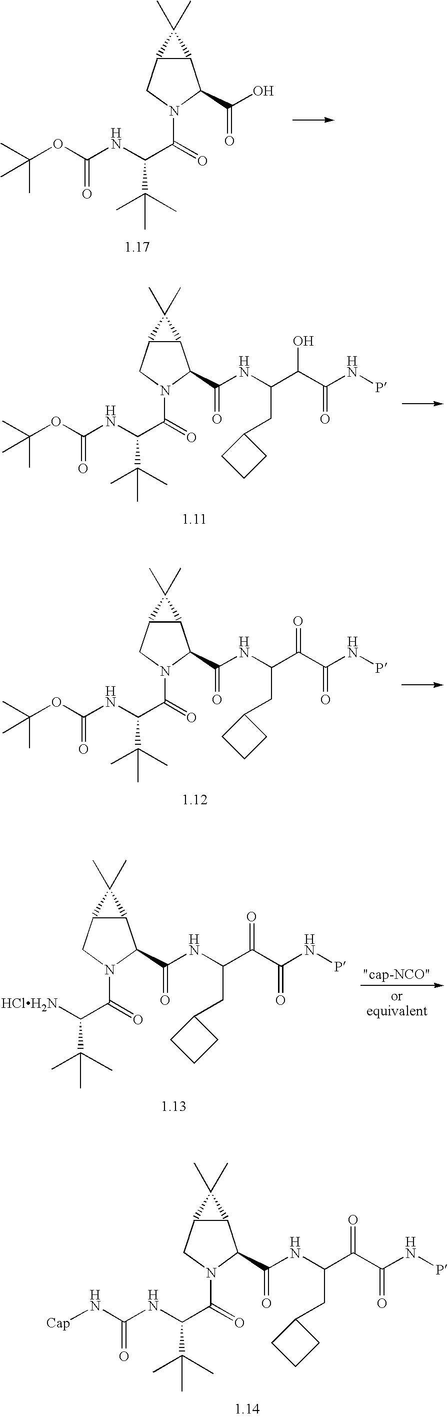
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 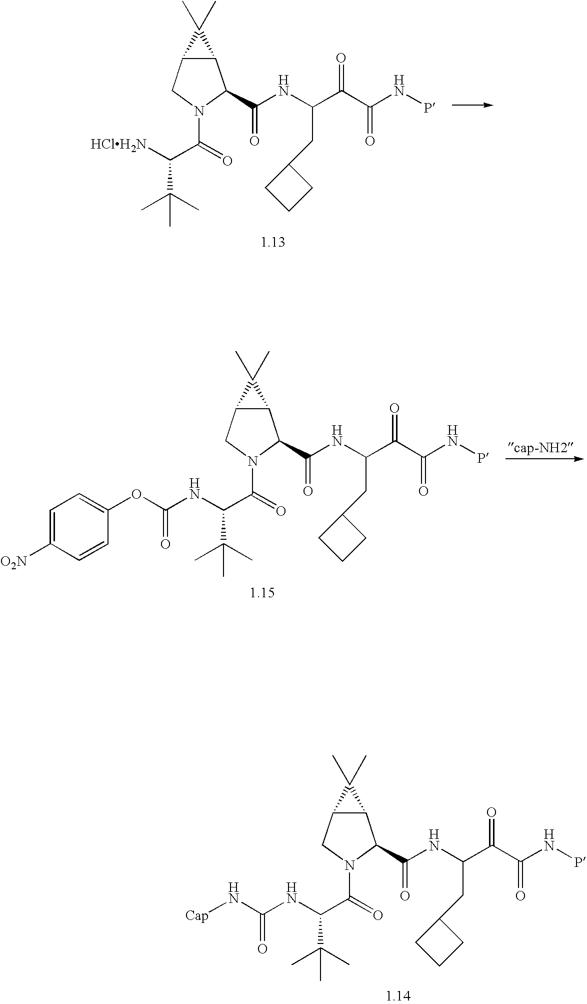
Method E

In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14. 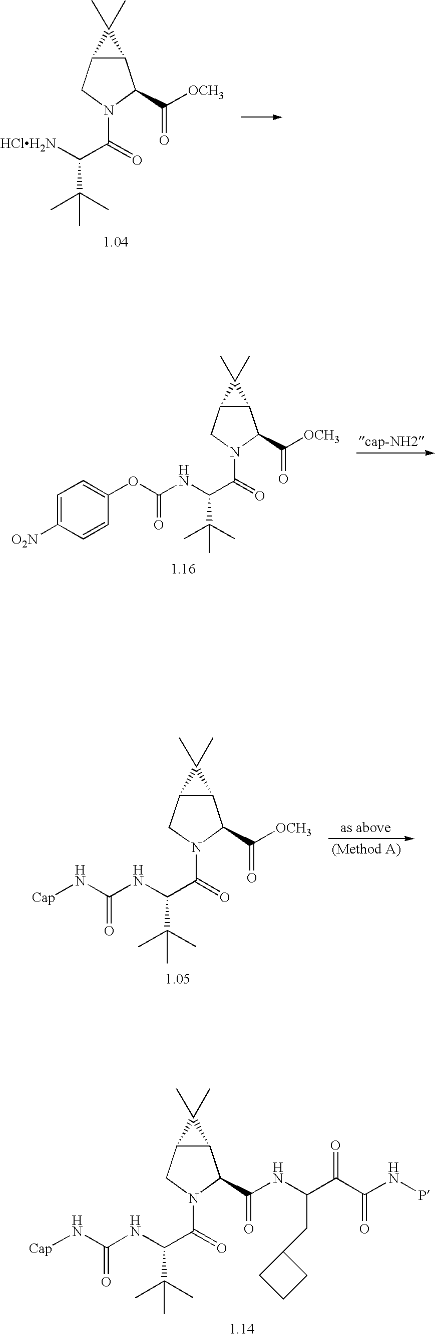
The Following Experimental Section Applies for the Preparation of the Compounds of Formula XVIII:

### Example 3

### Preparation of Compound of Formula 3

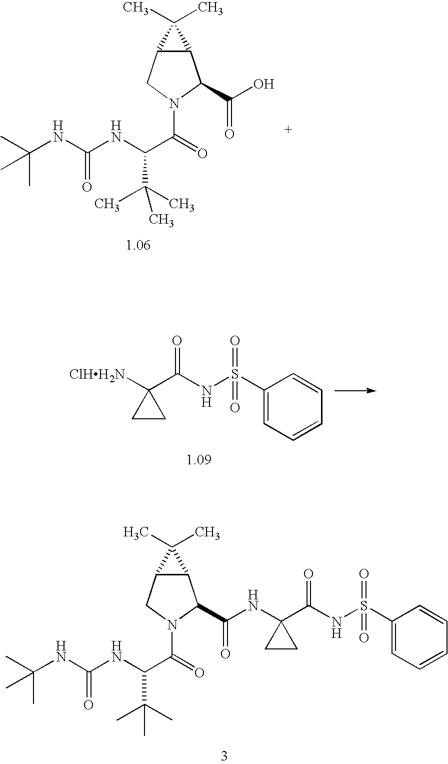

To a cooled solution (0° C.) of the intermediates 1.06 (75.0 mg, 0.2 mmol) and 1.09 (100.0 mg, 0.36 mmol) in DMF (5.0 mL) was added HATU (Aldrich, 76.05 mg, 0.20 mmol), followed by DIPEA (0.102 mL, 6 mmol). The reaction mixture was stirred for two days then warmed up to room temperature, diluted with ethyl acetate (40.0 mL), washed with 5% KH₂PO₄ containing 0.05 vol. of 1M H₃PO₄ and brine. Organic layer was dried over MgSO₄, filtered and concentrated to dryness. Residue was purified over silica gel using acetone —CH₂Cl₂ (1:9 to 1:1) to get 8.0 mg of product of formula 3 (6.5% yield); LCMS : (590.1).

One skilled in the art would understand that other suitable compounds of Formula XVIII can be prepared in a similar manner to that disclosed above.

The Following Experimental Section Applies for the Preparation of the Compounds of Formula XIX:

### Synthesis of Preparative Examples

### Synthesis of Example 101

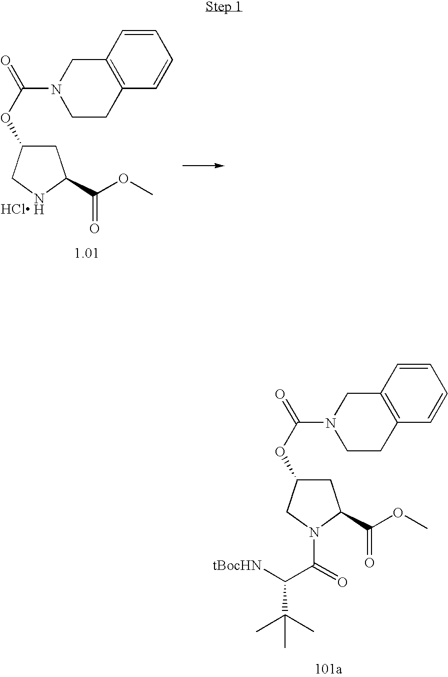

To a stirred solution of the proline derivative 1.01 (3.66 mmol, prepared as described above) in dichloromethane (20 mL) and DMF (15 mL) at 0° C. was added L-boc-tert-leucine (930 mg, 4.03 mmol), DIPEA (2.02 mL, 10.98 mmol) and HATU (1.8 g, 4.76 mmol). After 15 minutes at that temperature, the reaction flask was stored in the freezer (−20° C.), overnight (16 hr). The reaction mixture was diluted with dichloromethane (80 mL) and washed with saturated sodium bicarbonate solution (80 mL), 10% aq. citric acid solution (80 mL), brine (80 mL), dried (Na₂SO₄), filtered and concentrated. The crude material was purified by silica chromatography using 25/75 to 50/50 EtOAc/hexanes to provide 1.77 g of the required material, 101a. LC-MS: 518.1 (M+H)⁺.

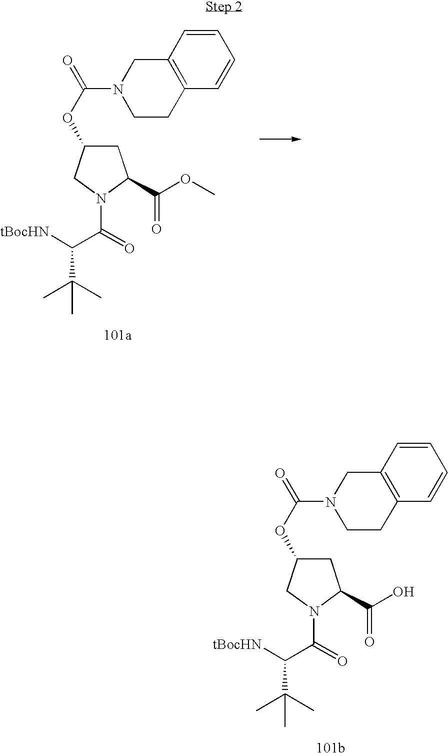

To a solution of the methyl ester 101a (1.21 g, 2.34 mmol) in THF (10 mL) and MeOH (5 mL) was added aq. 1M LiOH solution (5 mL). The reaction mixture was stirred at RT for 4 h. It was then concentrated, diluted with water (50 mL) and acidified with solid citric acid (pH approximately 3) when white solid material crashed out. This solid was filtered off, washed with water and dried in vacuo to afford 970 mg of 101b. LC-MS: 504.1 (M+H)⁺.

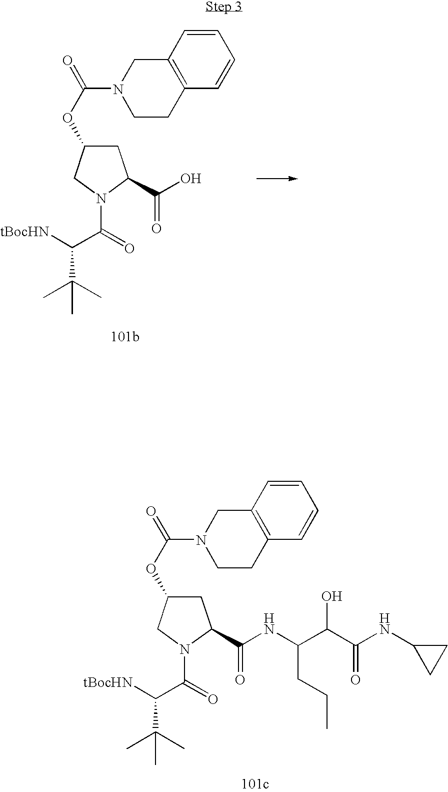

The acid 101b (503 mg, 1 mmol) was coupled with intermediate 13.06 (334 mg, 1.5 mmol) using essentially procedure described above (Step 1, preparation of 101a) to provide 101c which was used without purification. MS: 672.37 (M+H)⁺.

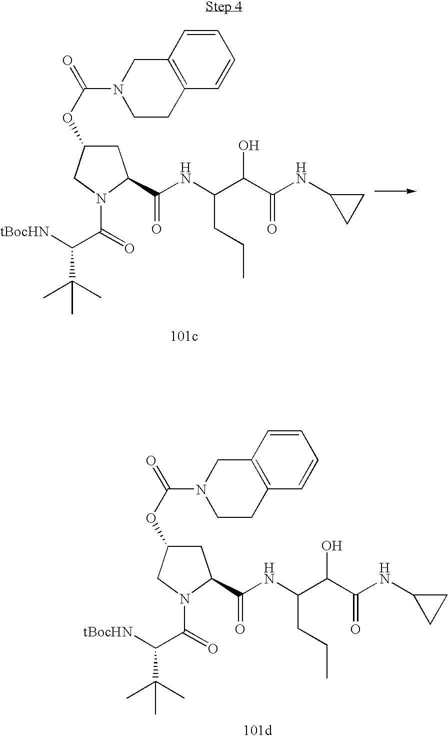

To a solution of the hydroxyl compound 101c from above in dichloromethane (15 mL) was added Dess-Martin's periodinane (848 mg, 2 mmol) and the reaction mixture was stirred at RT for 5 h. At this time, the reaction mixture was diluted with dichloromethane (30 mL) and washed with 1:1 mixture of aq. 10% sodium thiosulfate solution and saturated sodium bicarbonate solution (2×25 mL each), brine (50 mL), dried (Na₂SO₄), filtered and concentrated. The crude material was purified by silica chromatography using 15/85 to 50/50 acetone/hexanes to provide 410 mg of the required material, 101d. LC-MS: 670.2 (M+H)⁺.

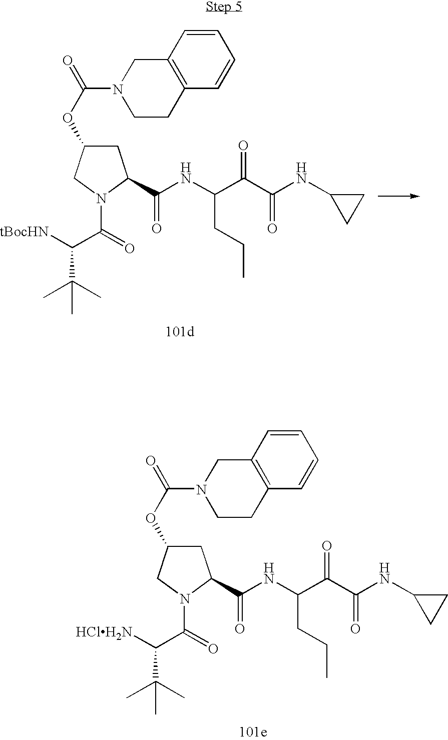

Deprotection of the N-boc functionality of 101d to provide the required material 101e was carried out as described for intermediate 1.01, Step 3 (reaction time =2 h). LC-MS: 570.1 (M+H)⁺.

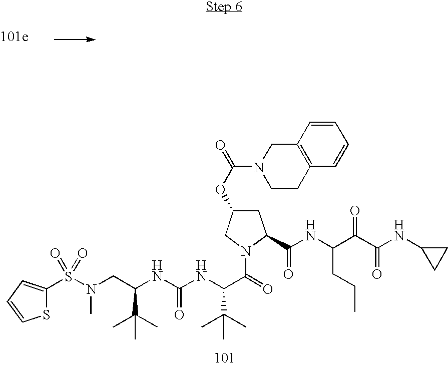

To a solution of the amine salt 101e (60 mg, 0.1 mmol) in dichloromethane (2 mL) at 0° C. was added DIPEA (0.06 mL, 0.3 mmol) followed by the isocyanate intermediate 65.01 (0.25 M solution in toluene, 0.8 mL, 0.2 mmol). After 15 minutes at that temperature, the reaction flask was stored in the freezer (−20° C.), overnight (16 hr). The reaction mixture was diluted with dichloromethane (20 mL) and washed with saturated ammonium chloride solution (20 mL), brine (20 mL), dried (Na₂SO₄), filtered and concentrated. The crude material was purified by silica chromatography using 15/85 to 50/50 acetone/hexanes to provide the required compound 101 (53 mg); LC-MS: 872.2 (M+H)⁺.

One skilled in the art would understand that other suitable compounds of Formula XIX can be prepared in a similar manner to that disclosed above.

The Following Experimental Section Applies for the Preparation of the Compounds of Formulae Ia, Ib and Ic:

Abbreviations:

Abbreviations which are used in the descriptions of the schemes, preparations and the examples that follow are:
THF: Tetrahydrofuran
DMF: N,N-Dimethylformamide
EtOAc: Ethyl acetate
AcOH: Acetic acid
HOOBt: 3-Hydroxy-1,2,3-benzotriazin-4(3H)-one
EDCl: 1-(3-dimethylaminopropyl)-3-ethylcarbodiimide hydrochloride
NMM: N-Methylmorpholine
MeOH: Methanol
EtOH: Ethanol
Et2O: Diethyl ether
DMSO: Dimethylsulfoxide
K^{t}BuO: Potassium tert-butoxide
DCM: Dichloromethane
Chg: Cyclohexylglycine
Bn: Benzyl
Et: Ethyl
Ph: Phenyl
iPr: isopropyl
^{t}Bu or Bu^{t}: tert-Butyl
Boc: tert-Butyloxycarbonyl
Cbz: Benzyloxycarbonyl
HATU: O-(7-azabenzotriazol-1-yl)-1,1,3,3-tetramethyluronium hexafluorophosphate
BOP: Benzotriazol-1-yl-oxy-tris(dimethylamino)hexafluorophosphate
10% Pd/C: 10% Palladium on carbon (by weight).

### Example

### Synthesis of (1R,5S)-N-[3-Amino-1-(Cyclobutylmethyl)-2,3-Dioxopropyl]-3-[2(S)-[[[(1,1-Dimethylethyl)Amino]Carbonyl]Amino]-3,3-Dimethyl-1-Oxobutyl]-6,6-Dimethyl-3-Azabicyclo[3.1.0]Hexan-2(S)-Carboxamide (Structure Ia)

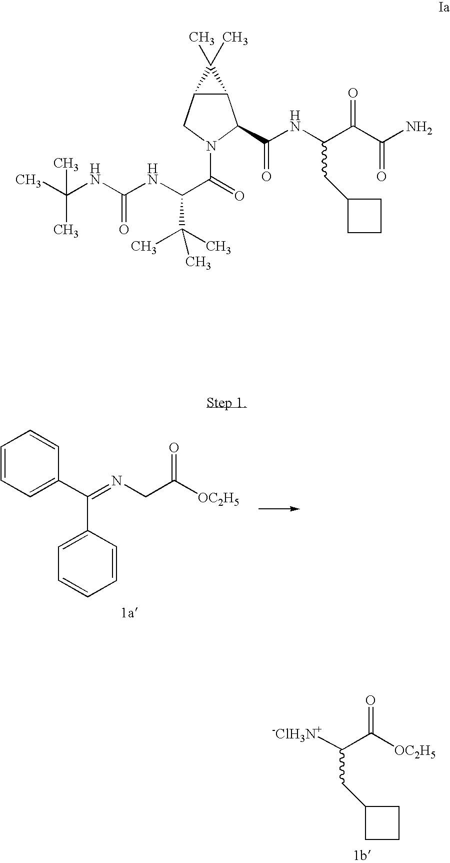

A stirred solution of the ketimime 1a′ (50 g, 187.1 mmol, available from Aldrich Chemical Company, Milwaukee, Wis.) under N₂ in dry THF (400 mL) was cooled to −78° C. and treated with 1 M solution of K-^{t}BuO (220 mL, 1.15 equiv.) in THF. The reaction mixture was warmed to 0° C. and stirred for 1 h and treated with bromomethylcyclobutane (28 mL, 249 mmol). The reaction mixture was stirred at room temperature for 48 h and concentrated in vacuo. The residue was dissolved in Et₂O (300 mL) and treated with aq. HCl (2 M, 300 mL) The resulting solution was stirred at room temperature for 5 h and extracted with Et₂O (1 L). The aqueous layer was made basic to pH˜12-14 with aq. NaOH (50%) and extracted with CH₂Cl₂ (3×300 mL). The combined organic layers were dried (MgSO₄), filtered, and concentrated to give pure amine (1b′, 18 g) as a colorless oil.

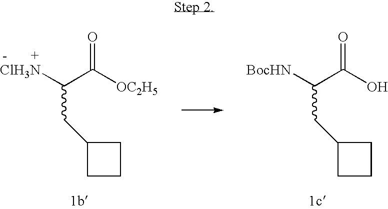

A solution of the amine 1b′ (18g, 105.2 mmol) at 0° C. in CH₂Cl₂ (350 mL) was treated with di-tert-butyidicarbonate (23 g, 105.4 mmol) and stirred at rt. for 12 h. After the completion of the reaction (TLC), the reaction mixture was concentrated in vacuo and the residue was dissolved in THF/H₂O (200 ml, 1:1) and treated with LiOH.H₂O (6.5 g, 158.5 mmol) and stirred at room temperature for 3 h. The reaction mixture was concentrated and the basic aqueous layer was extracted with Et₂O. The aqueous layer was acidified with conc. HCl to pH˜1-2 and extracted with CH₂Cl₂. The combined organic layers were dried (MgSO₄), filtered, and concentrated in vacuo to yield 1c′ as a colorless viscous oil which was used for next step without any further purification.

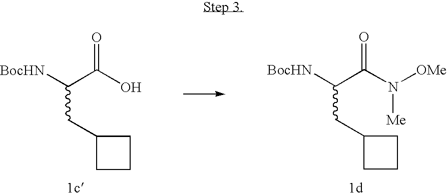

A solution of the acid 1c′ (15.0 g, 62 mmol) in CH₂Cl₂ (250 mL) was treated with BOP reagent (41.1 g, 93 mmol), N-methylmorpholine (27 mL), N,O-dimethyl hydroxylamine hydrochloride (9.07 g, 93 mmol) and stirred overnight at rt. The reaction mixture was diluted with 1 N aq. HCl (250 mL), and the layers were separated and the aqueous layer was extracted with CH₂Cl₂ (3×300 ml). The combined organic layers were dried (MgSO₄), filtered, concentrated in vacuo and purified by chromatography (SiO₂, EtOAc/Hex 2:3) to yield the amide 1d (15.0 g) as a colorless solid.

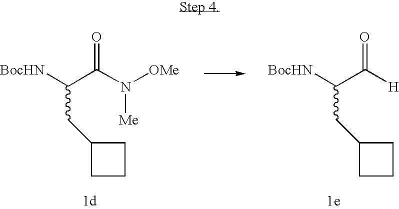

A solution of the amide 1d (15 g, 52.1 mmol) in dry THF (200 mL) was treated dropwise with a solution of LiAlH₄ (1 M, 93 mL, 93 mmol) at 0° C. The reaction mixture was stirred at room temperature for 1 h and carefully quenched at 0° C. with a solution of KHSO₄ (10% aq.) and stirred for 0.5 h. The reaction mixture was diluted with aq. HCl (1 M, 150 mL) and extracted with CH₂Cl₂ (3×200 mL), The combined organic layers were washed with aq. HCl (1 M), saturated NaHCO₃, brine, and dried (MgSO₄). The mixture was filtered and concentrated in vacuo to yield le as viscous colorless oil (14 g).

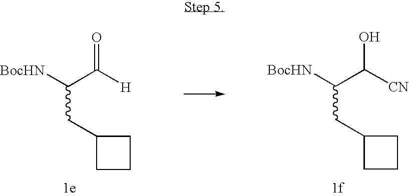

A solution of the aldehyde 1e (14 g, 61.6 mmol) in CH₂Cl₂ (50 mL), was treated with Et₃N (10.73 mL, 74.4 mmol), and acetone cyanohydrin (10.86 g, 127.57 mmol) and stirred at room temperature for 24 hrs. The reaction mixture was concentrated in vacuo and diluted with aq. HCl (1 M, 200 mL) and extracted into CH₂Cl₂ (3×200 mL). The combined organic layer were washed with H₂O, brine, dried (MgSO₄), filtered, concentrated in vacuo and purified by chromatography (SiO₂, EtOAc/Hex 1:4) to yield 1f (10.3 g) as a colorless liquid as a mixture of diastereomers.

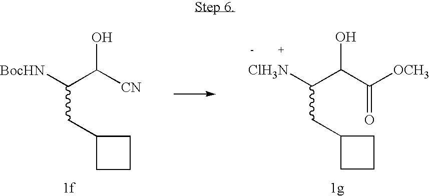

Methanol saturated with HCl*, prepared by bubbling HCl gas to CH₃OH (700 ml) at 0° C., was treated with cyanohydrin 1f and heated to reflux for 24 h. The reaction was concentrated in vacuo to yield 1g, which was used in the next step without purification.
* Alternatively 6M HCl prepared by addition of AcCl to dry methanol can also be used.

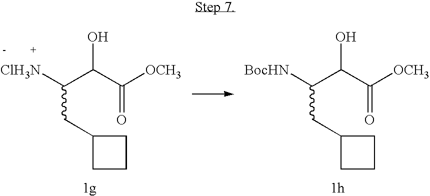

A solution of the amine hydrochloride 1g in CH₂Cl₂ (200 mL) was treated with Et₃N (45.0 mL, 315 mmol) and Boc₂O (45.7g, 209 mmol) at −78° C. The reaction mixture was then stirred at room temperature overnight and diluted with HCl (2 M, 200 mL) and extracted into CH₂Cl₂. The combined organic layers were dried (MgSO₄) filtered, concentrated in vacuo and purified by chromatography (EtOAc/Hex 1:4) to yield hydroxy ester 1 h.

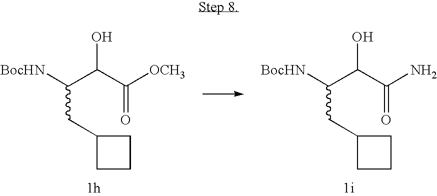

A solution of methyl ester 1 h (3g, 10.5 mmol) in THF/H₂O (1:1) was treated with LiOH.H₂O (645 mg, 15.75 mmol) and stirred at rt. for 2 h. The reaction mixture was acidified with aq HCl (1 M, 15 mL) and concentrated in vacuo. The residue was dried in vacuum.

A solution of the acid in CH₂Cl₂ (50 mL) and DMF (25 mL) was treated with NH₄Cl (2.94 g, 5.5 mmol), EDCl (3.15 g, 16.5 mmol), HOOBt (2.69 g, 16.5 mmol), and NMM (4.4 g, 44 mmol). The reaction mixture was stirred at room temperature for 3 d. The solvents were removed under vacuo and the residue was diluted with aq. HCl (250 mL) and extracted with CH₂Cl₂. The combined organic layers were washed with aq. saturated NaHCO₃, dried (MgSO₄) filtered concentrated in vacuo to obtain 1i, which was used as it is in the following steps. (Alternatively 1i can also be obtained directly by the reaction of 1f (4.5 g, 17.7 mmol) with aq. H₂O₂ (10 mL), LiOH.H₂O (820 mg, 20.8 mmol) at 0° C. in 50 mL of CH₃OH for 0.5 h.)

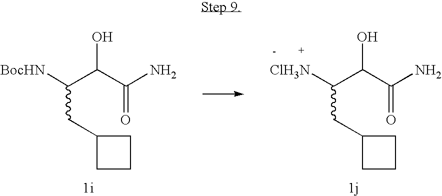

A solution of 1i obtained in the previous step was dissolved in 4 N HCl in dioxane and stirred at rt. for 2 h. The reaction mixture was concentrated in vacuo to give 1j as a solid, which was used without further purification.

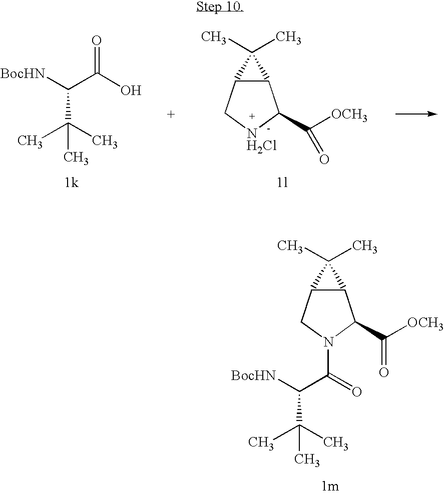

The amino ester 1l was prepared following the method of R. Zhang and J. S. Madalengoitia (J. Org. Chem. 1999, 64, 330), with the exception that the Boc group was cleaved by the reaction of the Boc-protected amino acid with methanolic HCl.

A solution of Boc-tert-Lue 1k (Fluka, 5.0 g 21.6 mmol) in dry CH₂Cl₂/DMF (50 mL, 1:1) was cooled to 0° C. and treated with the amine 1l (5.3 g, 25.7 mmol), NMM (6.5 g, 64.8 mmol) and BOP reagent (11.6 g, 25.7 mmol). The reaction was stirred at rt. for 24 hrs, diluted with aq. HCl (1 M) and extracted with CH₂Cl₂. The combined organic layers were washed with HCl (aq, 1 M), saturated NaHCO₃, brine, dried (MgSO₄), filtered and concentrated in vacuo and purified by chromatography (SiO₂, acetone/hexane 1:5) to yield 1m as a colorless solid.

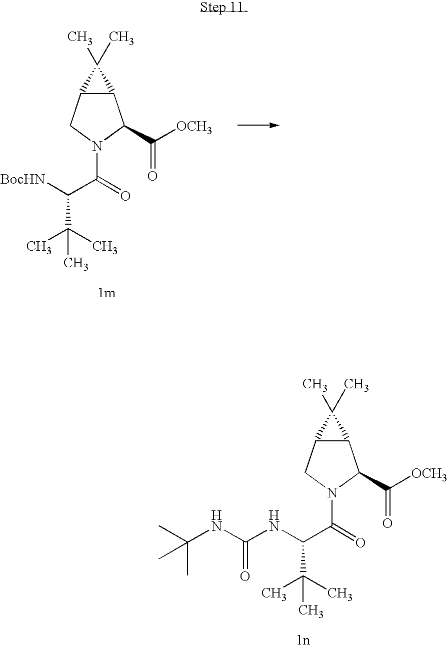

A solution of methyl ester 1m (4.0 g, 10.46 mmol) was dissolved in HCl (4 M solution in dioxane) and stirred at rt. for 3 h. The reaction mixture was concentrated in vacuo to obtain the amine hydrochloride salt used in the next step without further purification.

A solution of the amine hydrochloride salt (397 mg, 1.24 mmol) in CH₂Cl₂ (10 mL) was cooled to −78° C. and treated with tert-butyl isocyanate (250 mg, 2.5 mmol) and stirred at rt. overnight. The reaction mixture was concentrated in vacuo and the residue was diluted with aq. HCl (1M) and extracted with CH₂Cl₂. The combined organic layers were washed with aq. HCl (1M), saturated NaHCO₃ and brine. The organic layers were dried, filtered and concentrated in vacuo and the residue was purified by chromatography (SiO₂, acetone/Hex 1:4) to yield 1n as a colorless solid.

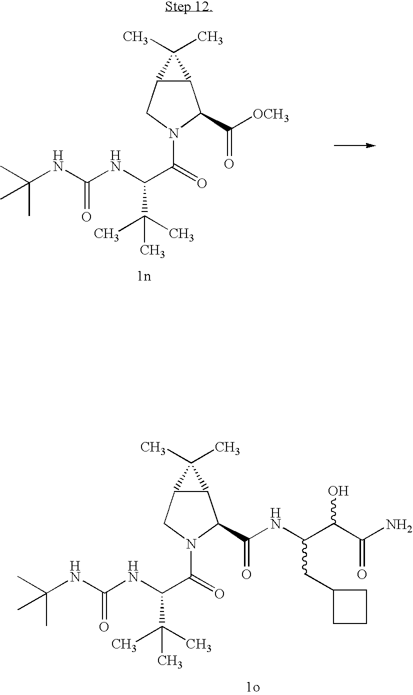

A solution of methyl ester 1n (381 mg, 1.0 mmol) in THF/H₂O (1:1, 5 mL) was treated with LiOH.H₂O (62 mg, 1.5 mmol) and stirred at rt. for 3 h. The reaction mixture was acidified with aq. HCl and concentrated in vacuo to obtain the free acid.

A solution of acid (254.9 mg, 0.69 mmol) in DMF/CH₂Cl₂ (1:1, 5.0 mL) was treated with amine 1j (159 mg, 0.763 mmol), EDCl (199 mg, 1.04 mmol), HOOBt (169.5 mg, 1.04 mmol) and NMM (280 mg, 2.77 mmol) at −20° C. The reaction mixture was stirred at −20° C. for 48 h and concentrated in vacuo. The residue was diluted with aq. 1 M HCl and extracted with EtOAc, The combined organic layers were extracted with aq. NaHCO₃, aq. HCl, brine, dried (MgSO₄) filtered, concentrated in vacuo to obtain 1o (470 mg) as a tan colored solid that was used in the next reaction without further purification.

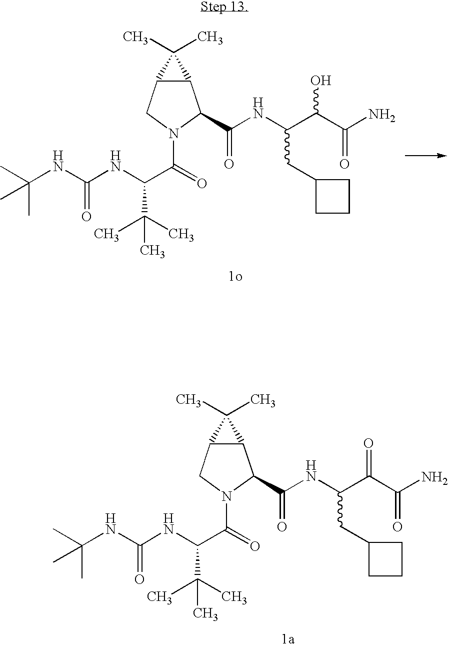

A solution of amide 1o (470 mg, 0.9 mmol) in toluene and DMSO (1:1 20 mL) at 0° C. was treated with EDCl (1.72 g, 9.0 mmol) and dichloroacetic acid (0.37 mL, 4.5 mmol) and stirred at 0° C. for 4 hrs. The reaction mixture was diluted with CH₂Cl₂, and washed with saturated NaHCO₃, and brine. The organic layer was dried (MgSO₄), filtered, concentrated, in vacuo and purified by chromatography (SiO₂, acetone/hexanes 3:7) to yield 1a as a colorless solid.
Separation of the Compound of Formula 1 into Diastereomers of Formulas Ib and Ic:

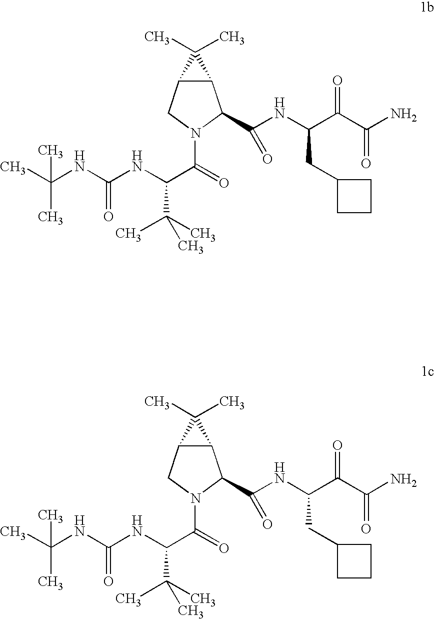

### Preparative HPLC Condition for Separation

COLUMN USED: NORMAL PHASE YMC DIOL-NP COLUMN 120 Å, S-10/20; 50 mm×500 mm I.D/length
SOLVENT A: Hexanes
SOLVENT B: To make 4 L of solvent (1.7 L Isopropanol +300 mL of CH₃CN+2 L of CH₂Cl₂)
HPLC CONDITIONS: 12% of Solvent B/88% of Solvent A
FLOW: 120 mL/min

Procedure: 1 g of compound 1a was dissolved in 10 mL of CH₂Cl₂/25 mL of Hexanes and injected into the column. It was eluted with 120 mL/min and two peaks were independently collected and concentrated. The solid residue was further dried in high vacuum and analyzed by analytical HPLC. Since the polar (second isomer) contained 2.6% of nonpolar diastereomer (First isomer), it was purified once more to isolate the pure diastereomers.

### Analytical Conditions for Analysis of Diastereomeric Purity

COLUMN USED: NORMAL PHASE YMC DIOL-NP COLUMN 200 Å, S-5 □M; 150 mm×3 mm length/I.D
SOLVENT A: Hexanes
SOLVENT B: To make 4 L of solvent (1.7 L Isopropanol +300 mL of CH₃CN+2 L of CH₂Cl₂)
HPLC CONDITIONS: 8.5% of Solvent B/91.5% of Solvent A
FLOW: 0.7 mL/min
Rt Nonpolar isomer (compound Ib)=13.2 min Polar isomer (compound Ic)=16.1 min

2.5 mg of compound in 1 mL was used and 20 μL was injected and analyzed with a U.V detector at λ=254 nm.

Analytical Data for Compounds 2 and 3.

Compound 3 [Polar Diastereomer]

¹H NMR (d₆-dmso, 500 MHz): δ 8.26 (d, 1 H, J=7.0 Hz), 8.00 (s, 1 H), 7.75 (s, 1 H), 5.96 (s, 1 H), 5.84 (d, 1 H, J=10 Hz), 4.96 (m, 1 H), 4.28 (s, 1 H), 4.11 (d, 1 H, J=11 Hz), 3.94 (d, 1H, J=10 Hz), 3.73 (dd, 1 H, J=10 & 5 Hz), 2.48 (m, 1 H), 1.95 (m, 2 H), 1.61 (m, 1 H), 1.59 (m, 1 H), 1.77(m, 1 H), 1.57 (m, 1 H), 1.74 (m, 2 H), 1.42 (dd, 1 H, J=7.5 & 5 Hz), 1.28 (d, 1 H, J=7.5 Hz), 1.17 (s, 9 H), 1.01 (s, 3 H), 0.90 (s, 9 H), 0.85 (s, 3 H). ¹³C NMR (d₆-dmso, 125 MHz): δ 197.8, 170.9, 170.8, 162.8, 157.4, 59.1, 56.8, 51.8, 48.9, 47.4, 36.7, 34.0, 32.0, 30.6, 29.1, 27.8, 27.3, 27.1, 26.4, 26.1, 18.5, 17.7, 12.5. MS [FAB] 520 (55), 421 (100), 308 (75), 213 (90). HRMS calcd for C₂₇H₄₆O₅N₅ [M+1]⁺520.3499. observed: 520.3505.

Compound 2 [Non-Polar Diastereomer]

¹H NMR (d₆-dmso, 500 MHz): δ 8.15 (d, 1 H, J=7.0 Hz), 7.96 (s, 1 H), 7.74 (s, 1 H), 5.96 (s, 1 H), 5.86 (d, 1 H, J=10 Hz), 4.85 (m, 1 H), 4.27 (s, 1H), 4.13 (d, 1 H, J=11.0 Hz), 3.97 (d, 1H, J=10 Hz), 3.76 (dd, 1 H, J=10 & 5 Hz), 2.36 (m, 1 H), 1.97 (m, 2 H), 1.60 (m, 2 H), 1.78 (m, 1 H), 1.64 (m, 1 H), 1.75 (m, 2 H), 1.44 (dd, 1 H, J=7.5 & 5 Hz), 1.27 (d, 1 H, J=7.5 Hz), 1.17 (s, 9 H), 1.00 (s, 3 H), 0.89 (s, 9 H), 0.82 (s, 3 H). ¹³C NMR (d₆-dmso125 MHz): δ 197.1, 171.1, 170.7, 163.0, 157.3, 59.4, 56.9, 52.1, 48.9, 47.4, 36.6, 34.0, 32.1, 30.5, 29.1, 27.9, 27.4, 26.8, 26.4, 26.1, 18.5, 17.8, 12.4. MS [FAB] 520 (40), 421 (100), 308 (60), 213 (65). HRMS calcd. for C₂₇H₄₆O₅N₅ [M+1]⁺520.3499. observed: 520.3514.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Each document (including granted patents, published patent applications, and nonpatent publications such as journal articles) referred to in this application is incorporated in its entirety by reference for all purposes.

What is claimed is:

1. A method for preventing, ameliorating or treating one or more symptoms of Hepatitis C virus (HCV) in a subject, wherein the method comprises administering to a subject in need of such treatment a dosage formulation containing at least one HCV protease inhibitor in a pharmaceutically effective amount thereof, wherein the dosage formulation is capable of maintaining an average Cmin plasma concentration of the at least one HCV protease inhibitor at or above 10 ng/ml,
wherein the at least one HCV protease inhibitor is selected from the group consisting of compounds of Formulae I to XXVI below:
a. Formula I 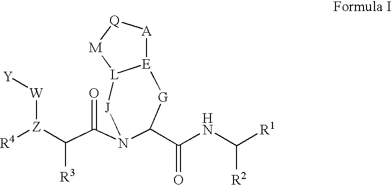
or a pharmaceutically acceptable salt, solvate or ester thereof,
wherein in Formula I above:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is H, OH, OR⁸, NR⁹R¹⁰, CF₃, C₂F₅, C₃F₇, CF₂R⁶, R⁶, or COR⁷ wherein R⁷ is H, OH, OR⁸, CHR⁹R¹⁰, or NR⁹R¹⁰, wherein R⁶, R⁸, R⁹ and R¹⁰ are independently selected from the group consisting of H, alkyl, aryl, heteroalkyl, heteroaryl, cycloalkyl, cycloalkyl, arylalkyl, heteroarylalkyl, [CH(R^{1′})]ₚCOOR¹¹, [CH(R^{1′})]ₚCONR¹²R¹³, [CH(R^{1′})]ₚSO₂R¹¹, [CH(R^{1′})]ₚCOR¹¹, [CH(R^{1′})]ₚCH(OH)R¹¹, CH(R^{1′})CONHCH(R²)COOR¹¹, CH(R^{1′})CONHCH(R^{2′})CONR¹²R¹³, CH(R^{1′})CONHCH(R²)R′, CH(R^{1′})CONHCH(R^{2′})CONHCH(R^{3′})COOR¹¹, CH(R^{1′})CONHCH(R^{2′})CONHCH(R^{3′})CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹ and CH(R^{1′})CONHCH(R^{2′})CONHCH(R^{3′})CONHCH(R^{4′})CONHCH(R^{5′}) CONR¹²R¹³, wherein R^{1′}, R^{2′}, R^{3′}, R^{4′}, R^{5′}, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;
Z is selected from O, N, CH or CR;
W maybe present or absent, and if W is present, W is selected from C═O, C═S, C(═N—CN), or SO₂;
Q maybe present or absent, and when Q is present, Q is CH, N, P, (CH₂)ₚ, (CHR)ₚ, (CRR′)ₚ, O, NR, S, or SO₂; and when Q is absent, M may be present or absent;
when Q and M are absent, A is directly linked to L;
A is O, CH₂, (CHR)ₚ, (CHR—CHR′)ₚ, (CRR′)ₚ, NR, S, SO₂ or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;
L may be present or absent, and when L is present, L is CH, CR, O, S or NR; and when L is absent, then M may be present or absent; and if M is present with L being absent, then M is directly and independently linked to E, and J is directly and independently linked to E;
M may be present or absent, and when M is present, M is O, NR, S, SO₂, (CH₂)ₚ, (CHR)ₚ(CHR—CHR′)ₚ, or (CRR′)ₚ;
p is a number from 0 to 6; and
R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen;
(cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring
b. Formula II 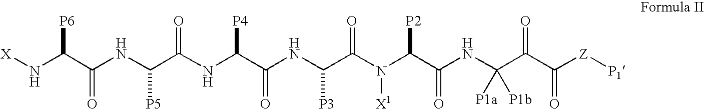
or a pharmaceutically acceptable salt, solvate or ester thereof;
wherein in Formula II above:
Z is O, NH or NR¹²;
X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylalkylcarbonyl, arylcarbonyl, heteroarylcarbonyl, alkoxycarbonyl, heterocyclyloxycarbonyl, aryloxycarbonyl, heteroaryloxycarbonyl, alkyaminocarbonyl, heterocyclylaminocarbonyl, arylaminocarbonyl, or heteroarylaminocarbonyl moiety, with the proviso that X may be additionally optionally substituted with R¹² or R¹³;
X¹ is H; C₁-C₄ straight chain alkyl; C₁-C₄ branched alkyl or; CH₂-aryl (substituted or unsubstituted);
R¹² is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R may be additionally optionally substituted with R¹³,
R¹³ is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³,
P1a, P1b, P2, P3, P4, P5, and P6 are independently:
H; C1-C10 straight or branched chain alkyl; C2-C10 straight or branched chain alkenyl;
C3-C8 cycloalkyl, C3-C8 heterocyclic; (cycloalkyl)alkyl or (heterocyclyl)alkyl wherein said cycloalkyl is made up of 3 to 8 carbon atoms, and zero to 6 oxygen, nitrogen, sulfur, or phosphorus atoms, and said alkyl is of 1 to 6 carbon atoms;
aryl, heteroaryl, arylalkyl, or heteroarylalkyl, wherein said alkyl is of 1 to 6 carbon atoms;
wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and
P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R¹³;
c. Formula III 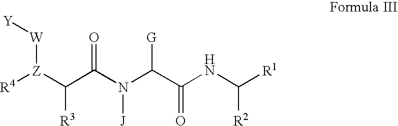
or a pharmaceutically acceptable salt, solvate or ester thereof;
wherein in Formula III above:
G, J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe additionally optionally substituted with X¹¹ or X¹²;
X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²; R¹ is COR⁵ or B(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl,
CH(R¹′)COOR¹¹, CH(R¹′)CONR¹²R¹³, CH(R¹′)CONHCH(R²′)COOR¹¹, CH(R^{1′})CONHCH(R²′)CONR¹²R¹³, CH(R¹′)CONHCH(R²′)R′, CH(R¹′)CONHCH(R²′)CONHCH(R³′)COOR¹¹, CH(R¹′)CONHCH(R²′)CONHCH(R³′)CONR¹²R¹³, CH(R¹′)CONHCH(R²′)CONHCH(R³′)CONHCH(R⁴′)COOR¹¹, CH(R¹′)CONHCH(R²′)CONHCH(R³′)CONHCH(R⁴′)CONR¹²R¹³, CH(R¹′)CONHCH(R²′)CONHCH(R³′)CONHCH(R⁴′)CONHCH(R⁵′)COOR¹¹, and CH(R¹′)CONHCH(R²′)CONHCH(R³′)CONHCH(R⁴′)CONHCH(R⁵′)CONR¹²R¹³, wherein R¹′, R²′, R³′, R⁴′, R⁵′, R¹¹, R¹², R¹³, and R′ may be the same or different and are independently selected from a group consisting of H, alkyl, aryl, heteroalkyl, heteroaryl, cycloalkyl, alkyl-aryl, alkyl-heteroaryl, aryl-alkyl and heteroaralkyl;
Z is selected from O, N, or CH;
W maybe present or absent, and if W is present, W is selected from C═O, C═S, or SO₂; and
R, R′, R², R³ and R⁴ are independently selected from the group consisting of H; C1-C10 alkyl; C2-C10 alkenyl; C3-C8 cycloalkyl; C3-C8 heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate;
d. Formula IV 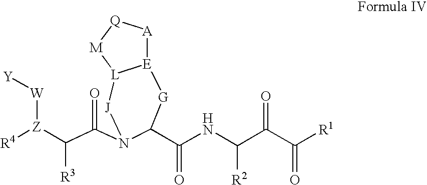
or a pharmaceutically acceptable salt, solvate or ester thereof;
wherein in Formula IV above:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is selected from the following structures: 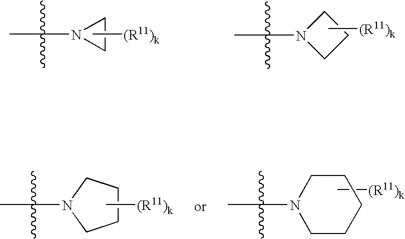
wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino,arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹≠H) maybe optionally substituted with X¹¹ or X¹²;
Z is selected from O, N, CH or CR;
W may be present or absent, and if W is present, W is selected from C═O, C═S, C(═N—CN), or S(O₂);
Q may be present or absent, and when Q is present, Q is CH, N, P, (CH₂)ₚ, (CHR)ₚ, (CRR′)ₚ, O, N(R), S, or S(O₂); and when Q is absent, M may be present or absent;
when Q and M are absent, A is directly linked to L;
A is O, CH₂, (CHR)ₚ, (CHR—CHR′)ₚ, (CRR′)ₚ, N(R), S, S(O₂) or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J may be present or absent, and when J is present, J is (CH₂)ₚ, (CHR)ₚ, or (CRR′)ₚ, S(O₂), NH, N(R) or O; and when J is absent, G is present and E is directly linked to N shown in Formula I as linked to J;
L may be present or absent, and when L is present, L is CH, C(R), O, S or N(R); and when L is absent, then M may be present or absent; and if M is present with L being absent, then M is directly and independently linked to E, and J is directly and independently linked to E;
M may be present or absent, and when M is present, M is O, N(R), S, S(O₂), (CH₂)ₚ, (CHR)ₚ(CHR—CHR′)ₚ, or (CRR′)ₚ;
p is a number from 0 to 6; and
R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered cyclic ring structure or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring;
e. Formula V 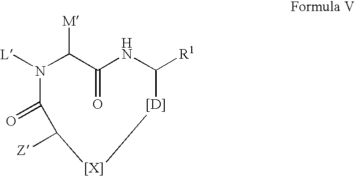
or a pharmaceutically acceptable salt, solvate or ester thereof,
wherein in Formula V above:
(1) R¹ is —C(O)R⁵ or —B(OR)₂;
(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;
(3) R⁷ is H, —OH, —OR⁸, or —CHR⁹R¹⁰;
(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R^{1′})CH(R^{1′})C(O)OR¹¹, [CH(R^{1′})]ₚC(O)OR¹¹, —[CH(R^{1′})]ₚC(O)NR¹²R¹³, —[CH(R^{1′})]ₚS(O₂)R¹¹, —[CH(R^{1′})]ₚC(O)R¹¹, —[CH(R^{1′})]ₚS(O₂)NR¹²R¹³, CH(R^{1′})C(O)N(H)CH(R^{2′})(R′), CH(R^{1′})CH(R^{1′})C(O)NR¹²R¹³, —CH(R^{1′})CH(R^{1′})S(O₂)R¹¹, —CH(R^{1′})CH(R^{1′})S(O₂)NR¹²R¹³, —CH(R^{1′})CH(R^{1′})C(O)R¹¹, —[CH(R^{1′})]ₚCH(OH)R¹¹, —CH(R^{1′})C(O)N(H)CH(R^{2′})C(O)OR¹¹, C(O)N(H)CH(R^{2′})C(O)OR¹¹, —C(O)N(H)CH(R^{2′})C(O)R¹¹, CH(R^{1′})C(O)N(H)CH(R^{2′})C(O)NR¹²R¹³, —CH(R^{1′})C(O)N(H)CH(R^{2′})R′, CH(R^{1′})C(O)N(H)CH(R^{2′})C(O)N(H)CH(R^{3′})C(O)OR¹¹, CH(R^{1′})C(O)N(H)CH(R^{2′})C(O)CH(R^{3′})NR¹²R¹³, CH(R^{1′})C(O)N(H)CH(R^{2′})C(O)N(H)CH(R^{3′})C(O)NR¹²R¹³, CH(R^{1′})C(O)N(H)CH(R^{2′})C(O)N(H)CH(R^{3′})C(O)N(H)CH(R^{4′})C(O)OR¹¹, CH(R^{1′})C(O)N(H)CH(R^{2′})C(O)N(H)CH(R^{3′})C(O)N(H)CH(R^{4′})C(O)NR¹²R¹³, CH(R^{1′})C(O)N(H)CH(R²)C(O)N(H)CH(R^{3′})C(O)N(H)CH(R^{4′})C(O)N(H)CH(R^{5′})C(O)OR¹¹, and CH(R^{1′})C(O)N(H)CH(R^{2′})C(O)N(H)CH(R^{3′})C(O)N(H)CH(R^{4′})C(O)N(H)CH(R^{5′})C(O)NR¹²R¹³; wherein R^{1′}, R^{2′}, R^{3′}, R^{4′}, R^{5′}, R¹¹, R¹² and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl; or
R¹² and R¹³ are linked together wherein the combination is cycloalkyl, heterocycloalkyl, ary or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain;
or L′ and M′ are linked together to form a ring structure wherein the portion of structural Formula 1 represented by 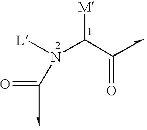
is represented by structural Formula 2: 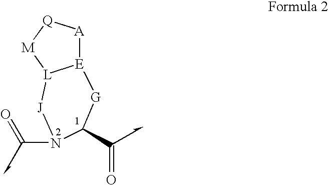
wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;
L is present or absent, and when L is present, L is C(H) or C(R); when L is absent, M is present or absent; if M is present with L being absent, then M is directly and independently linked to E, and J is directly and independently linked to E;
G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;
Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent being selected from —OR, —CH(R)(R′), S(O)₀₋₂R or —NRR′ or (iii) absent; when both Q and M are absent, A is either directly linked to L, or A is an independent substituent on E, said independent substituent bing selected from —OR, —CH(R)(R′), S(O)₀₋₂R or —NRR′ or A is absent;
A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, O, OR, N(R), S, S(O₂), (CH₂)ₚ, (CHR)ₚ(CHR—CHR′)ₚ, or (CRR′)ₚ; or M is linked to A to form an alicyclic, aliphatic or heteroalicyclic bridge;
(8) Z′ is represented by the structural Formula 3: 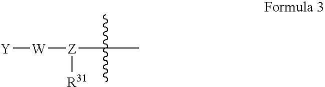
wherein in Formula 3, Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
Z is O, N, C(H) or C(R);
R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
W may be present or absent, and if W is present, W is C(═O), C(═S), C(═N—CN), or S(O₂);
(9) X is represented by structural Formula 4: 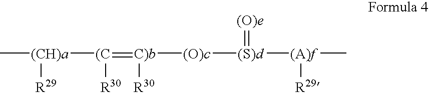
wherein in Formula 4, a is 2, 3, 4, 5, 6, 7, 8 or 9;
b, c, d, e and fare 0, 1, 2, 3, 4 or 5;
A is C, N, S or O;
R²⁹ and R^{29′} are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y¹ and Y₂ can be the same or different and are independently selected from the group consisting of hydrogen, alkyl, aryl, and aralkyl; or
R²⁹ and R^{29′} are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;
(10) D is represented by structural Formula 5: 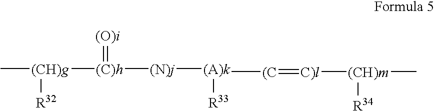
wherein in Formula 5, R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R³² and R³⁴ are linked together such that the combination forms a portion of a cycloalkyl group;
g is 1, 2, 3, 4, 5, 6, 7, 8 or 9;
h, i, j, k, l and m are 0, 1, 2, 3, 4 or 5; and
A is C, N, S or O,
(11) provided that when structural Formula 2: 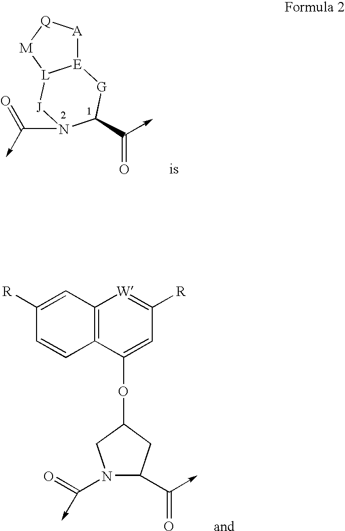
W′ is CH or N, both the following conditional exclusions (i) and (ii) apply:
conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆ alkyl or C₃₋₆ cycloalkyl; and
conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈ alkyl, C₃₋₆ cycloalkyl, C_{6 to 10} aryl or C₇₋₁₆ aralkyl;
f. Formula VI 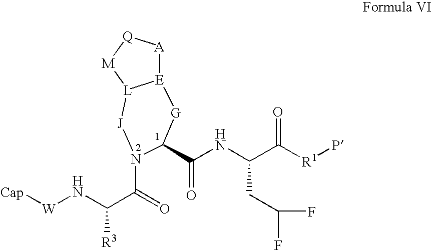
or a pharmaceutically acceptable salt, solvate or ester of said compound, wherein in Formula VI above:
Cap and P′ are independently H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino can be unsubstituted or optionally independently substituted with one or two substituents which can be the same or different and are independently selected from X¹ and X²;
X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;
X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;
W may be present or absent, and when W is present W is C(═O), C(═S), C(═NH), C(═N—OH), C(═N—CN), S(O) or S(O₂);
Q maybe present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R′), S(O)₀₋₂R or —NRR′; when both Q and M are absent, A is either directly linked to L, or A is an independent substituent on E, selected from —OR, CH(R)(R′), —S(O)₀₋₂R or —NRR′;
A is present or absent and if present A is —O—, —O(R)CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either Q and E are connected by a bond or Q is an independent substituent on M;
E is present or absent and if present E is CH, N, C(R);
G may be present or absent, and when G is present, G is (CH₂)ₚ, (CHR)ₚ, or (CRR′)ₚ; when G is absent, J is present and E is directly connected to the carbon atom marked position 1;
J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
L may be present or absent, and when L is present, L is CH, N, or CR; when L is absent, M is present or absent; if M is present with L being absent, then M is directly and independently linked to E, and J is directly and independently linked to E;
M may be present or absent, and when M is present, M is O, N(R), S, S(O₂), (CH₂)ₚ, (CHR)ₚ, (CHR—CHR′)ₚ, or (CRR′)ₚ;
p is a number from 0 to 6;
R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;
R and R′ in (CRR′) can be linked together such that the combination forms a cycloalkyl or heterocyclyl moiety; and
R¹ is N(R) or O;
g. Formula VII 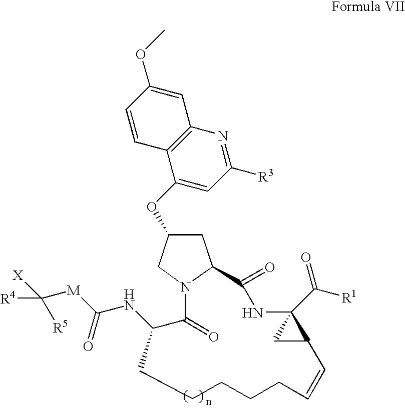
or a pharmaceutically acceptable salt, solvate or ester thereof, wherein in Formula VII above:
M is O, N(H), or CH₂;
n is 0-4;
R¹ is —OR⁶, —NR⁶R⁷ or 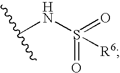
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 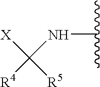
is represented by 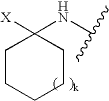
where k is 0 to 2;
X is selected from the group consisting of: 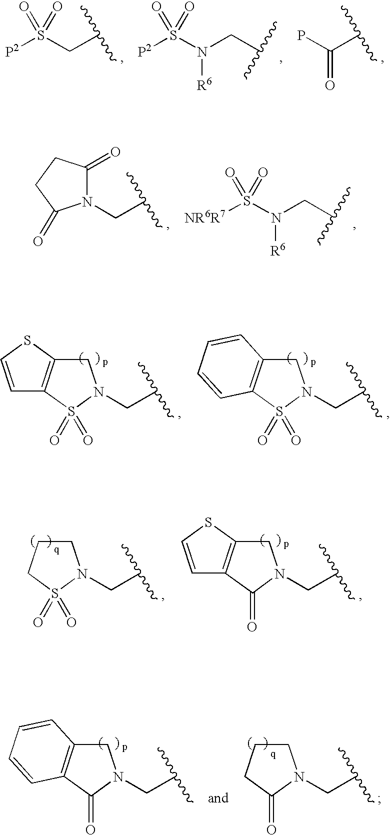
where p is 1 to 2, q is 1-3 and p² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 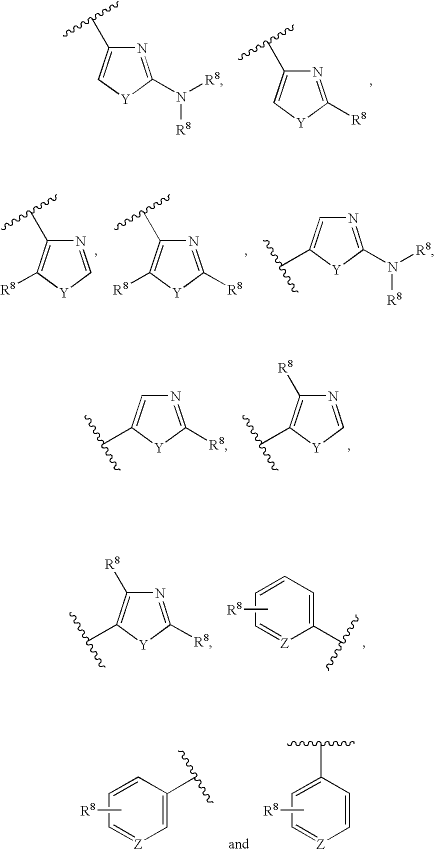
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
h. Formula VIII 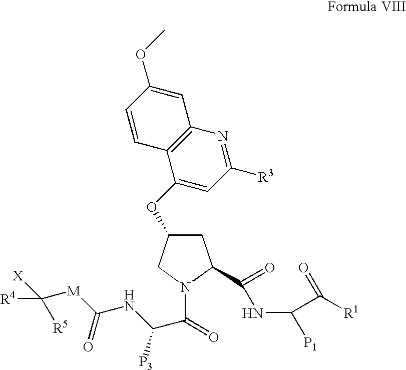
or a pharmaceutically acceptable salt, solvate or ester thereof, wherein in Formula VIII above,
M is O, N(H), or CH₂;
R¹ is —OR⁶, —NR⁶R⁷ or 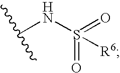
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
P₁ is selected from the group consisting of alkyl, alkenyl, alkynyl, cycloalkyl haloalkyl;
P₃ is selected from the group consisting of alkyl, cycloalkyl, aryl and cycloalkyl fused with aryl;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 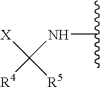
is represented by 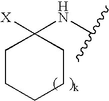
where k is 0 to 2;
X is selected from the group consisting of: 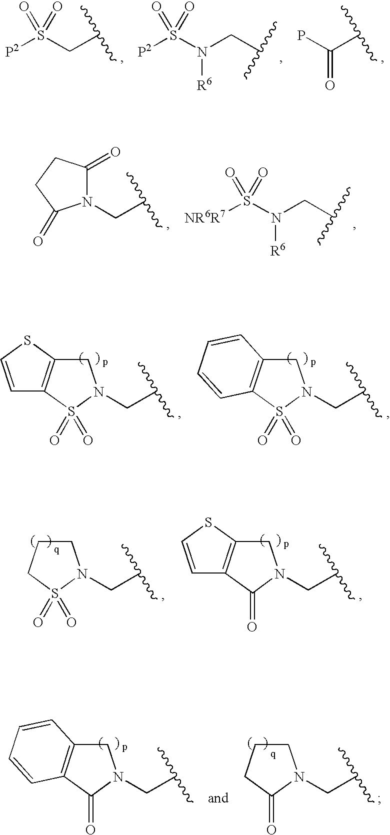
where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 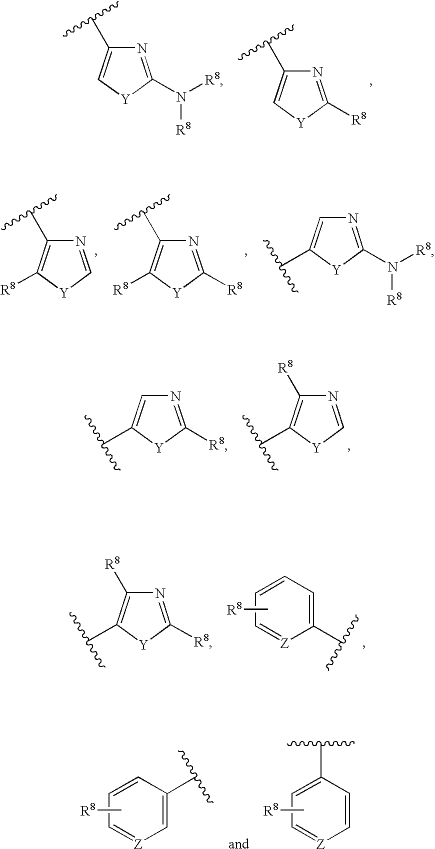
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
i. Formula IX 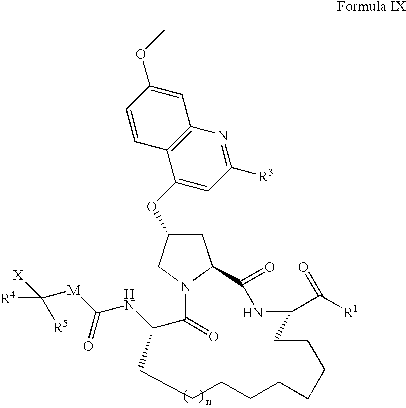
or a pharmaceutically acceptable salt, solvate or ester thereof, wherein in Formula IX above,
M is O, N(H), or CH₂;
n is 0-4;
R¹ is —OR⁶, —NR⁶R⁷ or 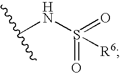
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 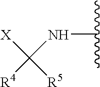
is represented by 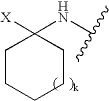
where k is 0 to 2;
X is selected from the group consisting of: 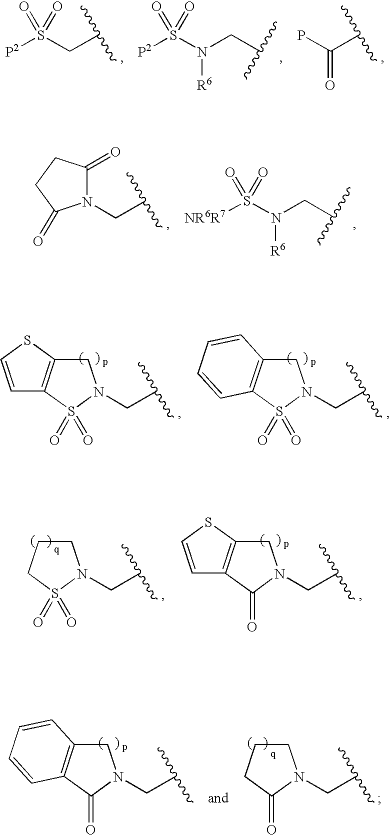
where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 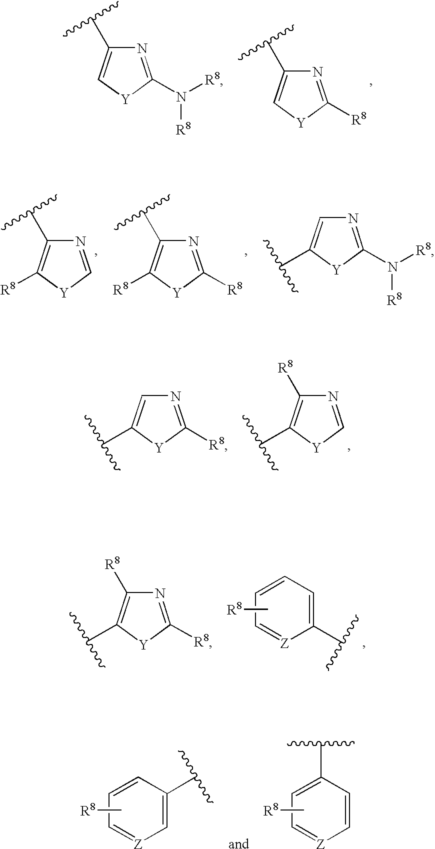
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
j. Formula X 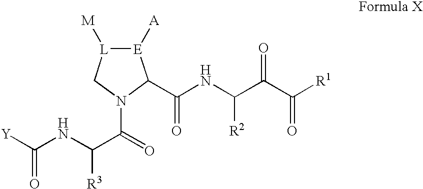
or a pharmaceutically acceptable salt, solvate or ester thereof;
wherein in Formula X above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 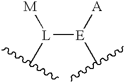
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 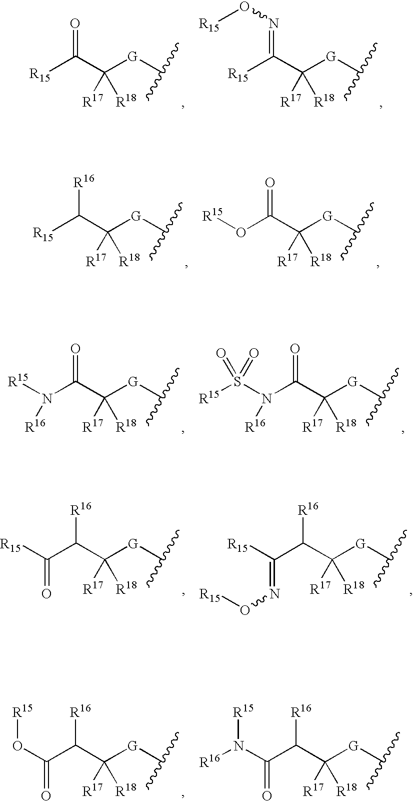
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
k. Formula XI 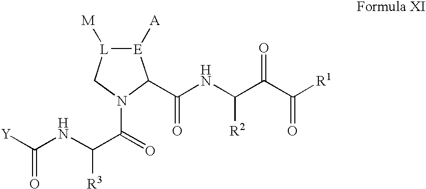
or a pharmaceutically acceptable salt, solvate or ester thereof; wherein in Formula XI above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, NR⁹R¹⁰, SR, SO₂R, and halo; or A and M are connected to each other (in other words, A-E-L-M taken together) such that the moiety: 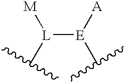
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;
Y is selected from the following moieties: 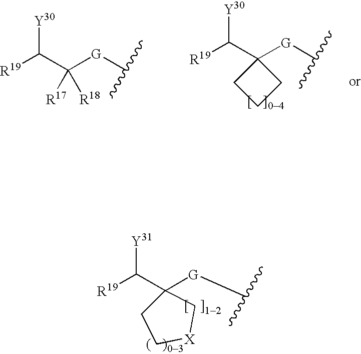
wherein y³⁰ and Y³¹ are selected from 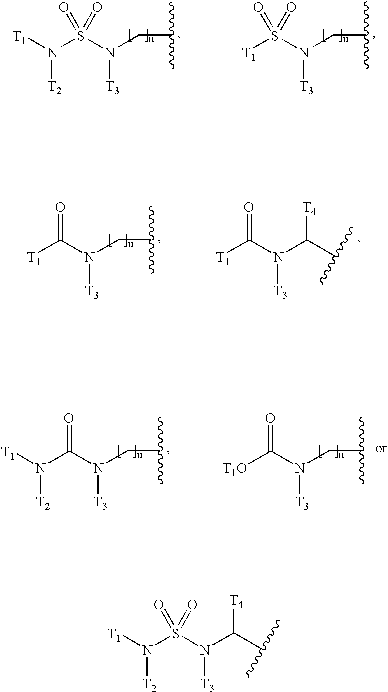
where u is a number 0-6;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, T₃ and T₄ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
l. Formula XII 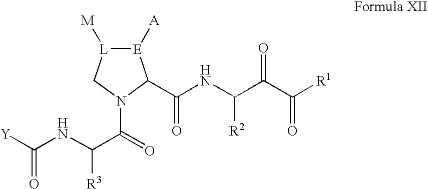
or a pharmaceutically acceptable salt, solvate or ester thereof; wherein in Formula XII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 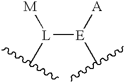
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 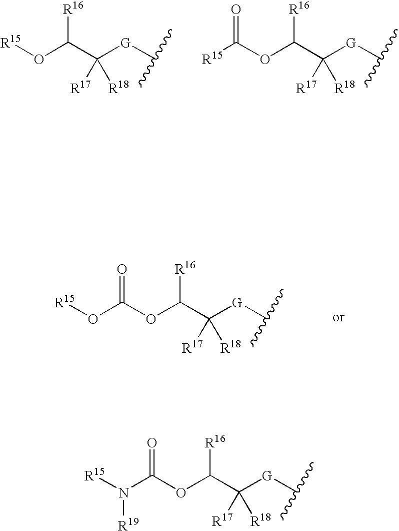
wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro;
m. Formula XIII 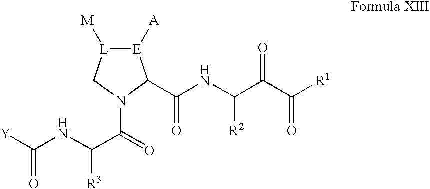
or a pharmaceutically acceptable salt, solvate or ester thereof; wherein in Formula XIII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other (in other words, A-E-L-M taken together) such that the moiety: 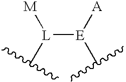
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 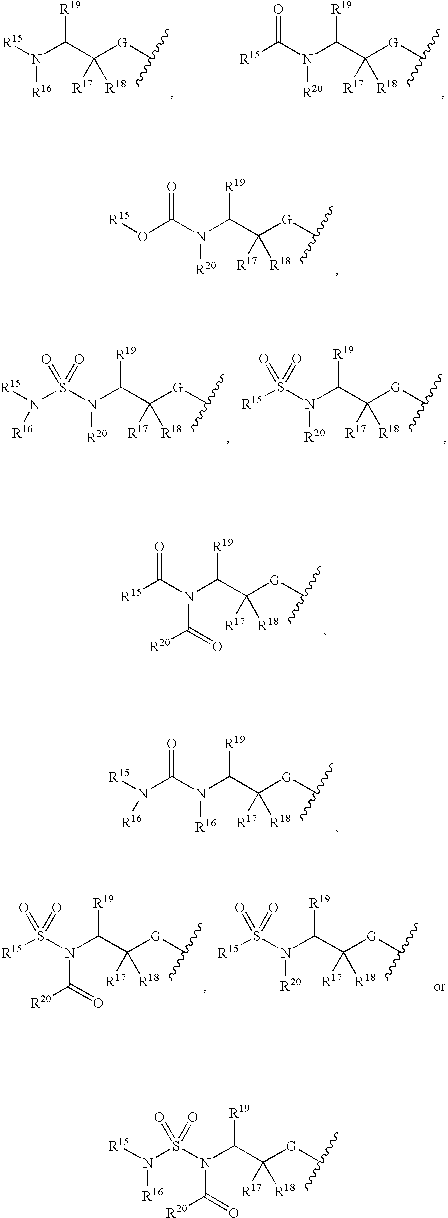
wherein G is NH or O, and R¹⁵, R¹⁶, R¹⁷, R¹⁸, R¹⁹ and R²⁰ can be the same or different, each being independently selected from the group consisting of H, C₁-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
n. Formula XIV 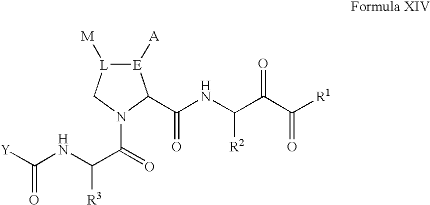
or a pharmaceutically acceptable salt, solvate or ester thereof; wherein in Formula XIV above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo;
or A and M are connected to each other such that the moiety: 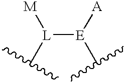
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;
and Y is selected from the following moieties: 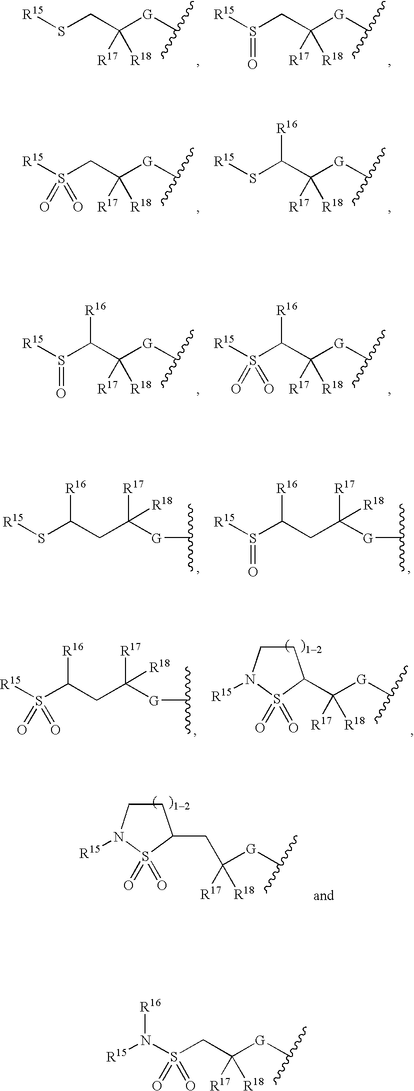
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro;
o. Formula XV 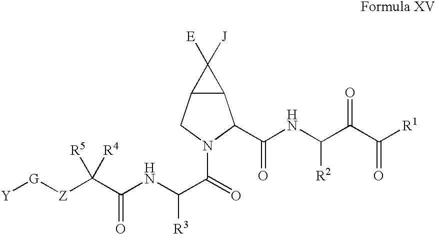
or a pharmaceutically acceptable salt, solvate or ester thereof; wherein in Formula XV above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, aryl-, heteroalkyl-, heteroaryl-, cycloalkyl-, cycloalkyl-, arylalkyl-, and heteroarylalkyl;
E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;
Z is N(H), N(R), or O, with the proviso that when Z is O, G is present or absent and if G is present with Z being O, then G is C(═O);
G maybe present or absent, and if G is present, G is C(═O) or S(O₂), and when G is absent, Z is directly connected to Y;
Y is selected from the group consisting of: 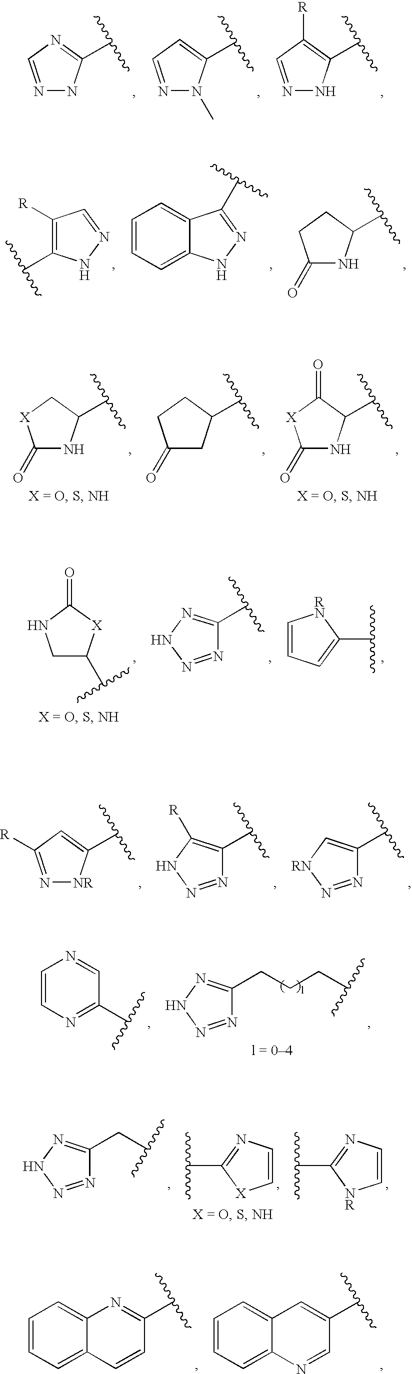 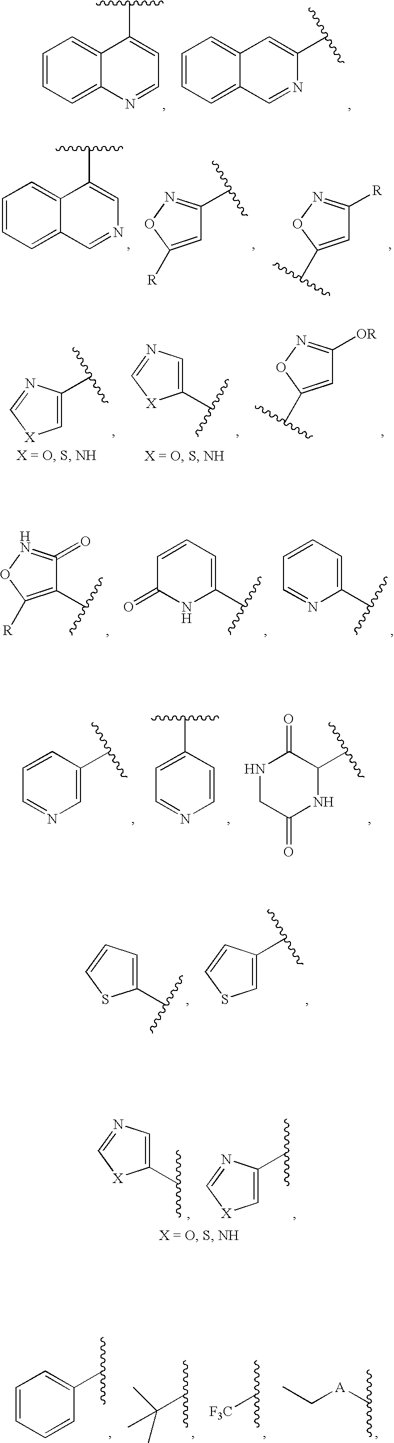 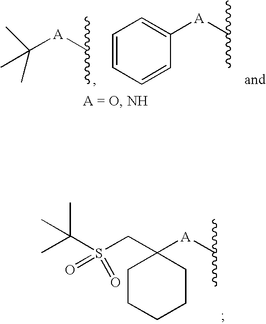
R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;
wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
p. Formula XVI 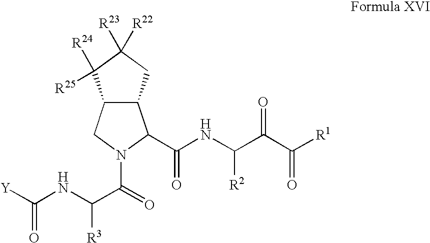
or a pharmaceutically acceptable salt, solvate or ester thereof; wherein in Formula XVI above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 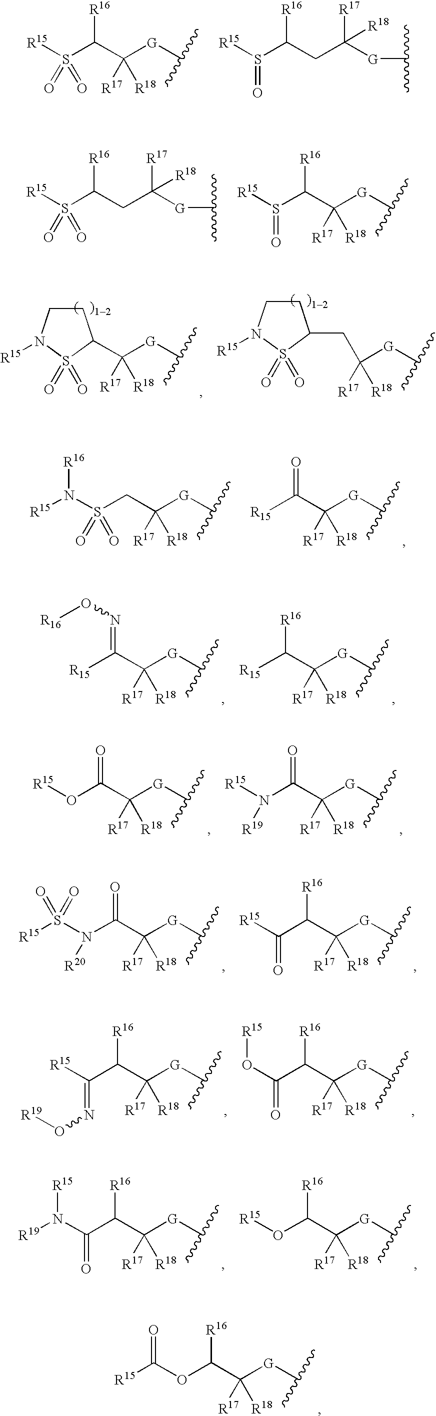 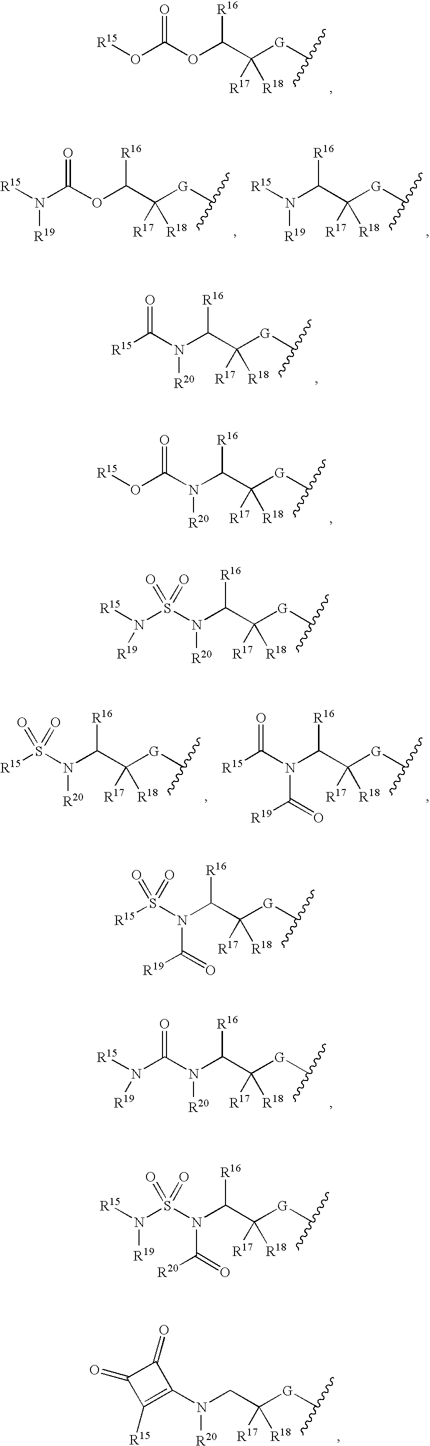 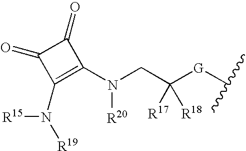
wherein G is NH or O and R¹⁵, R¹⁶, R¹⁷, R¹⁸, R¹⁹, R²⁰, R²¹, R²², R²³, R²⁴ and R²⁵ can be the same or different, each being independently selected from the group consisting of H, alkyl, heteroalkyl, alkenyl, heteroalkenyl alkynyl, heteroalkynyl, cycloalkyl, heterocyclyl, aryl, arylalkyl, heteroalkyl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
q. Formula XVII 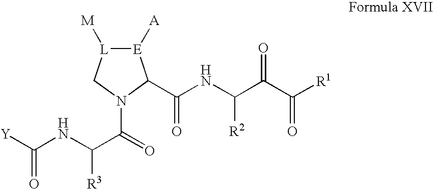
or a pharmaceutically acceptable salt, solvate or ester thereof; wherein in Formula XVII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 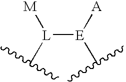
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
Y is selected from the following moieties: 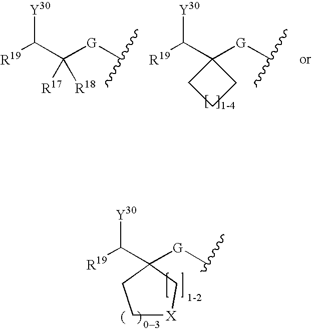
wherein Y³⁰ is selected from 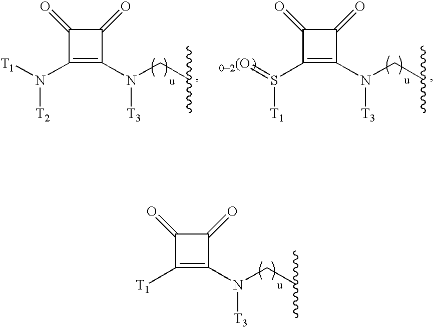
where u is a number 0-1;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
r. Formula XVIII 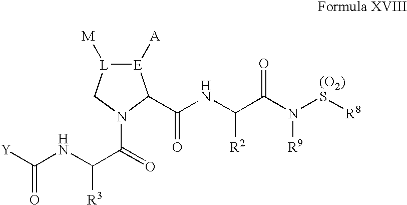
or a pharmaceutically acceptable salt, solvate or ester thereof, wherein in Formula XVIII above:
R⁸ is selected from the group consisting of alkyl-, aryl-, heteroalkyl-, heteroaryl-, cycloalkyl-, heterocyclyl-, arylalkyl-, heteroarylalkyl-, and heterocyclylalkyl;
R⁹ is selected from the group consisting of H, alkyl, alkenyl, alkynyl, aryl and cycloalkyl;
A and M can be the same or different, each being independently selected from R, OR, N(H)R, N(RR′), SR, S(O₂)R, and halo; or A and M are connected to each other (in other words, A-E-L-M taken together) such that the moiety: 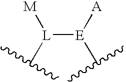
shown above in Formula I forms either a three, four, five, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl;
R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;
Y is selected from the following moieties: 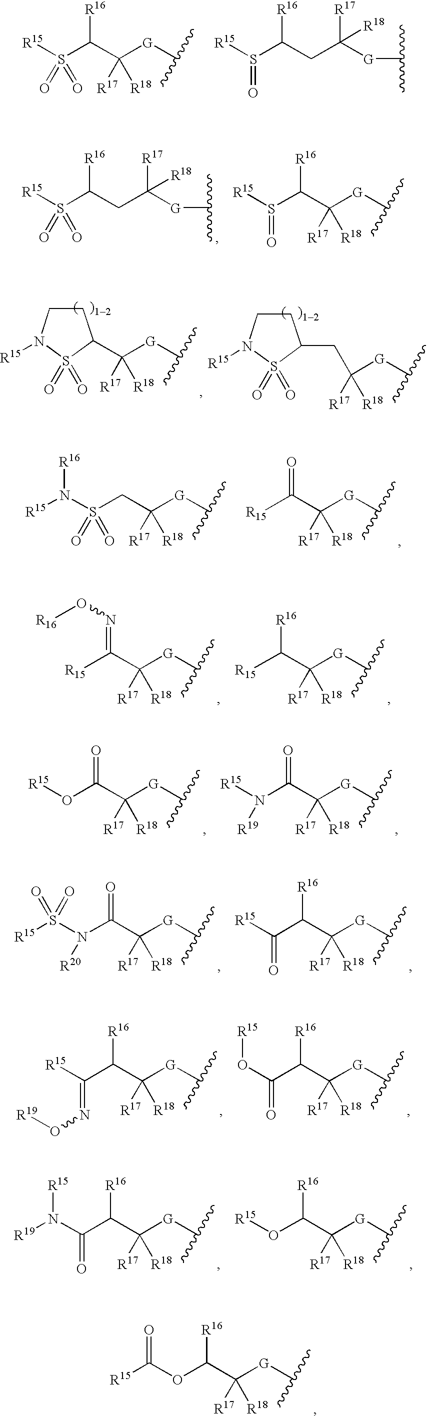 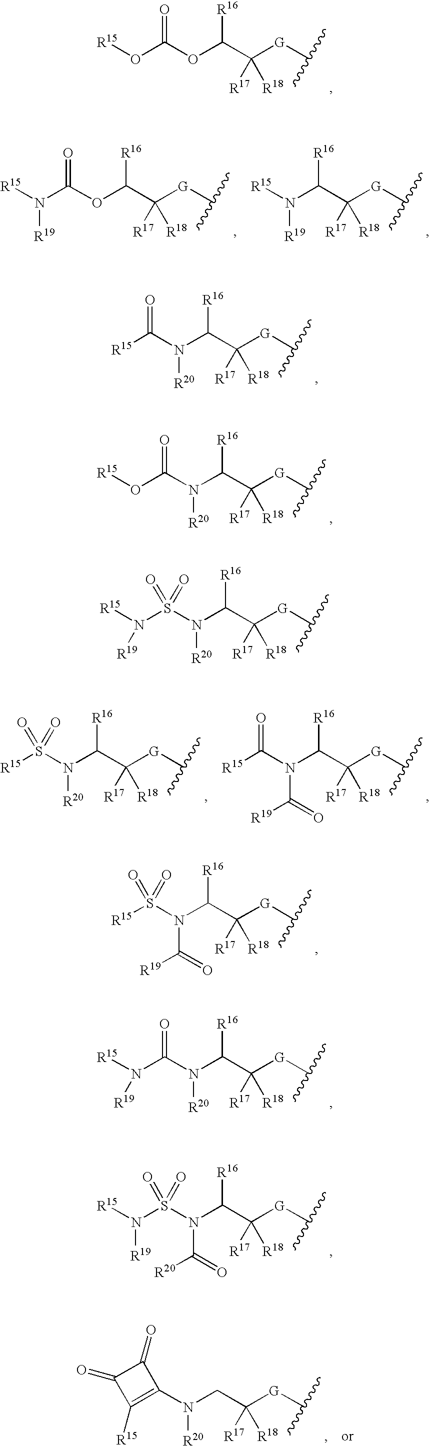 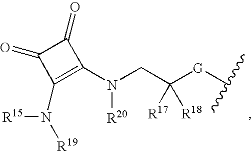
wherein G is NH or O; and R¹⁵, R¹⁶, R¹⁷, R¹⁸, R¹⁹ and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl, spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
s. Formula XIX 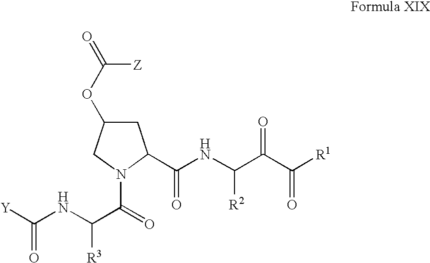
wherein in Formula XIX above:
Z is selected from the group consisting of a heterocyclyl moiety,
N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N(heteroaryl)₂;
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 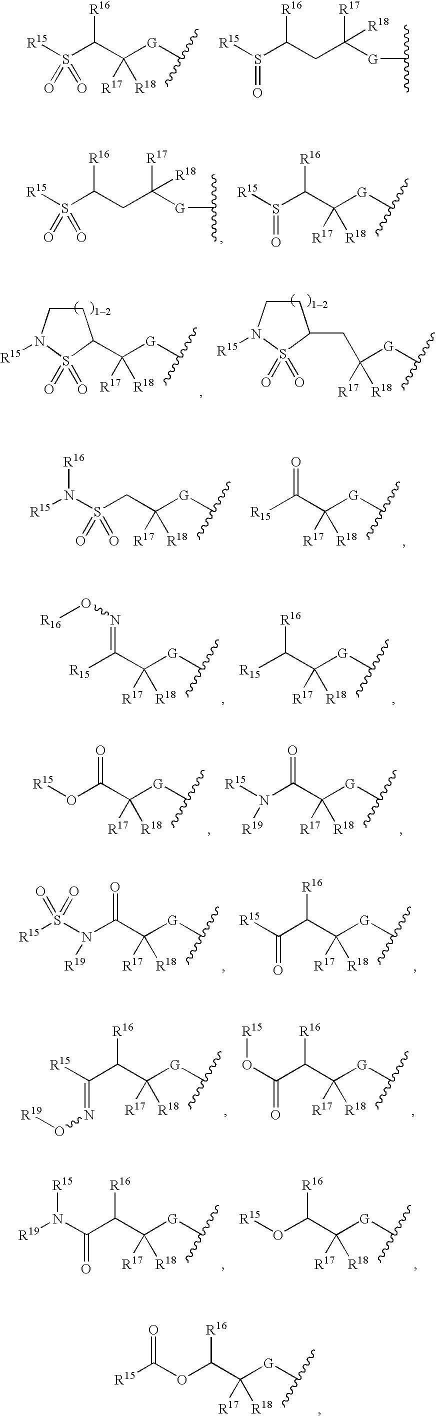 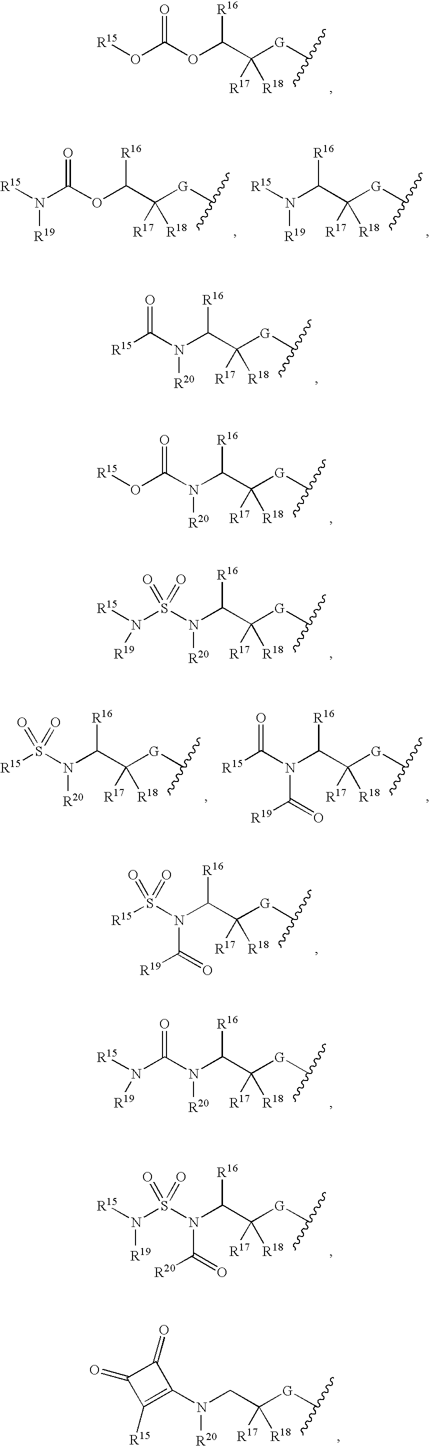 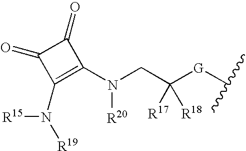
wherein G is NH or O; and R¹⁵, R¹⁶, R¹⁷, R¹⁸, R¹⁹, R²⁰ and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
t. Formula XX 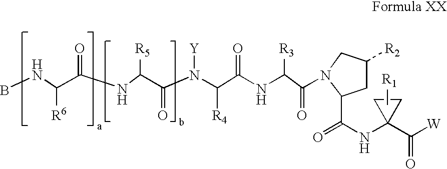
or a pharmaceutically acceptable salt, solvate or ester thereof; wherein in Formula XX above:
a is 0 or 1; b is 0 or 1; Y is H or C₁₋₆ alkyl;
B is H, an acyl derivative of formula R₇—C(O)— or a sulfonyl of formula R₇—SO2 wherein
R7 is (i) C₁₋₁₀ alkyl optionally substituted with carboxyl, C₁₋₆ alkanoyloxy or C₁₋₆ alkoxy;
(ii) C₃₋₇ cycloalkyl optionally substituted with carboxyl, (C₁₋₆ alkoxy)carbonyl or phenylmethoxycarbonyl;
(iii) C₆ or C₁₀ aryl or C₇₋₁₆ aralkyl optionally substituted with C₁₋₆ alkyl, hydroxy, or amino optionally substituted with C₁₋₆ alkyl; or
(iv) Het optionally substituted with C₁₋₆ alkyl, hydroxy, amino optionally substituted with C₁₋₆ alkyl, or amido optionally substituted with C₁₋₆ alkyl;
R₆, when present, is C₁₋₆ alkyl substituted with carboxyl;
R₅, when present, is C₁₋₆ alkyl optionally substituted with carboxyl;
R₄ is C₁₋₁₀ alkyl, C₃₋₇ cycloalkyl or C₄₋₁₀ (alkylcycloalkyl);
R₃ is C₁₋₁₀ alkyl, C₃₋₇ cycloalkyl or C₄₋₁₀ (alkylcycloalkyl);
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri-substituted with R₂₁,
or R₂₀ is Het or (lower alkyl)-Het optionally mono-, di- or tri-substituted with R₂₁, wherein each R₂₁ is independently C₁₋₆ alkyl; C₁₋₆ alkoxy; amino optionally mono- or di-substituted with C₁₋₆ alkyl; sulfonyl; NO₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;
wherein R₂₂ is C₁₋₆alkyl; C₁₋₆ alkoxy; amino optionally mono- or di-substituted with C₁₋₆ alkyl; sulfonyl; NO₂; OH; SH; halo; haloalkyl; carboxyl; amide or (lower alkyl)amide;
R₁ is C₁₋₆ alkyl or C₂₋₆ alkenyl optionally substituted with halogen; and
W is hydroxy or a N-substituted amino;
u. Formula XXl: 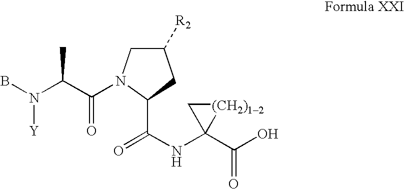
or a pharmaceutically acceptable salt, solvate or ester thereof; wherein in Formula XXI above:
B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl of formula R₄—O—C(O)—; an amide of formula R₄—N(R₅)—C(O)—; a thioamide of formula R₄—N(R₅)—C(S)—; or a sulfonyl of formula R₄—SO₂ wherein
R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;
(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;
(iii) amino optionally mono- or di-substituted with C₁₋₆ alkyl; amido; or (lower alkyl)amide;
(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or
(v) Het or (lower alkyl)-Het, both optionally substituted with C₁₋₆ alkyl, hydroxy, amido, (lower alkyl) amide, or amino optionally mono- or di-substituted with C₁₋₆ alkyl;
R₅ is H or C₁₋₆ alkyl;
with the proviso that when R₄ is an amide or a thioamide, R₄ is not (ii) a cycloalkoxy;
Y is H or C₁₋₆ alkyl;
R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,
or R₂₀ is Het or (lower alkyl)-Het, both optionally mono-, di- or tri-substituted with R₂₁,
wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;
wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;
R1 is H; C₁₋₆ alkyl, C₃₋₇ cycloalkyl, C₂₋₆ alkenyl, or C₂₋₆ alkynyl, all optionally substituted with halogen;
v. Formula XXII: 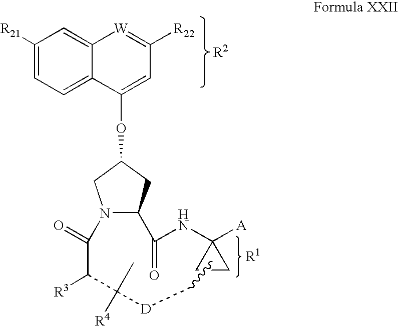
or a pharmaceutically acceptable salt, solvate or ester thereof; wherein in Formula XXII above:
W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂, wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵, wherein each R²⁵ is independently: H, C₁₋₆ alkyl or C₃₋₆ cycloalkyl;
or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹, wherein
R⁴¹ is H, C₁₋₆ alkyl, C₃₋₆ cycloalkyl or —C(O)—R⁴²,
wherein R⁴² is C₁₋₆ alkyl, C₃₋₆ cycloalkyl or C_{6 or 10} aryl;
R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C 1-6 thioalkyl, and A is an amide of formula —C(O)—NH—R⁵, wherein R⁵ is selected from the group consisting of: C₁₋₈ alkyl, C₃₋₆ cycloalkyl, C_{6 or 10} aryl and C₇₋₁₆ aralkyl;
or A is a carboxylic acid;
w. Formula XXIII: 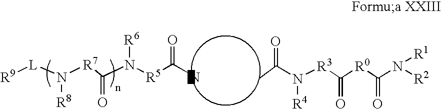
a pharmaceutically acceptable salt, solvate or ester thereof; wherein in Formula XXIII above:
R⁰ is a bond or difluoromethylene;
R¹ is hydrogen, optionally substituted aliphatic group, optionally substituted cyclic group or optionally substituted aromatic group;
R² and R⁹ are each independently optionally substituted aliphatic group, optionally substituted cyclic group or optionally substituted aromatic group;
R3, R5 and R7 are each independently:
optionally substituted (1,1- or 1,2-)cycloalkylene; or
optionally substituted (1,1- or 1,2-) heterocyclylene; or
methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or;
R4, R6, R8 and R¹⁰ are each independently hydrogen or optionally substituted aliphatic group; 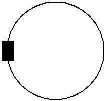
is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-(N(R⁸)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰OC(O)—, —S(O)₂—, or —NR¹⁰S(O)₂—; and n is 0 or 1, provided
when 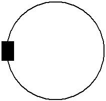
is substituted 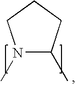
then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴ is optionally substituted aliphatic;
x. Formula XXIV: 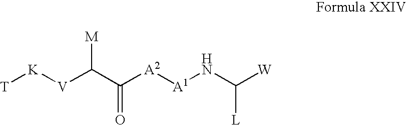
or a pharmaceutically acceptable salt, solvate or ester thereof; wherein in Formula XXIV above:
W is: 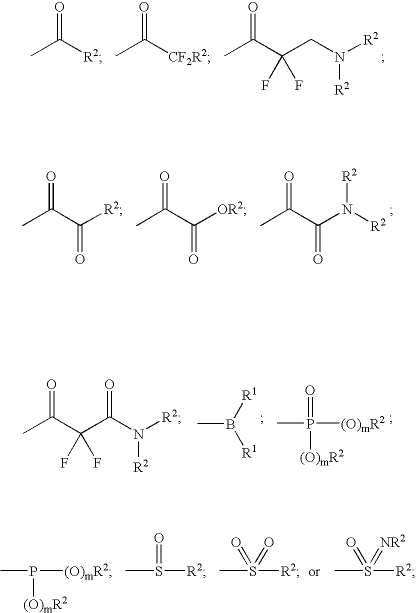
m is 0 or 1;
each R¹ is hydroxy, alkoxy, or aryloxy, or each R¹ is an oxygen atom and together with the boron, to which they are each bound, form a 5-7 membered ring, wherein the ring atoms are carbon, nitrogen, or oxygen;
each R² is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl, or two R² groups, which are bound to the same nitrogen atom, form together with that nitrogen atom, a 5-7 membered monocyclic heterocyclic ring system; wherein any R² carbon atom is optionally substituted with J;
J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;
J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoaikyl, halo, cyano, nitro, formyl, sulfonyl, or sulfonamido;
L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;
A¹ is a bond;
R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;
X is a bond, —C(H)(R7)-, —O—, —S—, or —N(R8)-;
R⁷ is hydrogen, alkyl, alkenyl, aryl, aralkyl, heterocyclyl, heterocyclylalkyl, heteroaryl, or heteroaralkyl, and is optionally substituted with 1-3 J groups;
R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R¹⁴, —SO₂R¹⁴, or carboxamido, and is optionally substititued with 1-3 J groups; or R⁸ and Z, together with the atoms to which they are bound, form a nitrogen containing mono- or bicyclic ring system optionally substituted with 1-3 J groups;
R¹⁴ is alkyl, aryl, aralkyl, heterocyclyl, heterocyclyalkyl, heteroaryl, or heteroaralkyl;
Y is a bond, —CH₂—, —C(O)—, —C(O)C(O)—, —S(O)—, —S(O)₂—, or —S(O)(NR⁷)—, wherein R⁷ is as defined above;
Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;
A² is a bond or 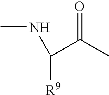
R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;
V is a bond, —CH₂—, —C(H)(R¹¹)—, —O—, —S—, or —N(R¹¹)—;
R¹¹ is hydrogen or C₁₋₃ alkyl;
K is a bond, —O—, —S—, —C(O)—, —S(O)—, —S(O)₂—, or —S(O)(NR¹¹)—, wherein R¹¹ is as defined above;
T is —R¹², -alkyl-R¹², -alkenyl-R¹², -alkynyl-R¹², —OR¹², —N(R¹²)2, —C(O)R¹², —C(═NOalkyl)R¹², or 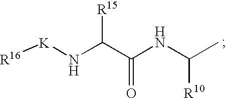
R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;
R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;
R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and
R¹⁶ is hydrogen, alkyl, aryl, heteroaryl, cycloalkyl, or heterocyclyl;
y. Formula XXV: 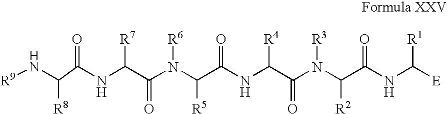
or a pharmaceutically acceptable salt, solvate or ester thereof;
wherein in Formula XVII above:
E represents CHO or B(OH)2;
R¹ represents lower alkyl, halo-lower alkyl, cyano-lower alkyl, lower alkylthio-lower alkyl, aryl-lower alkylthio-lower alkyl, aryl-lower alkyl, heteroaryllower alkyl, lower alkenyl or lower alkynyl;
R² represents lower alkyl, hydroxy-lower alkyl, carboxylower alkyl, aryl-lower alkyl, aminocarbonyl-lower alkyl or lower cycloalkyl-lower alkyl; and
R³ represents hydrogen or lower alkyl;
or R² and R³ together represent di- or trimethylene optionally substituted by hydroxy;
R4 represents lower alkyl, hydroxy-lower alkyl, lower cycloalkyl-lower alkyl, carboxy-lower alkyl, aryllower alkyl, lower alkylthio-lower alkyl, cyano-lower alkylthio-lower alkyl, aryl-lower alkylthio-lower alkyl, lower alkenyl, aryl or lower cycloalkyl;
R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;
R⁶ represents hydrogen or lower alkyl;
R⁷ represent lower alkyl, hydroxydower alkyl, carboxylower alkyl, aryl-iower alkyl, lower cycloalkyl-lower alkyl or lower cycloalkyl;
R⁸ represents lower alkyl, hydroxy-lower alkyl, carboxylower alkyl or aryl-lower alkyl; and
R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl; and
z. Formula XXVI: 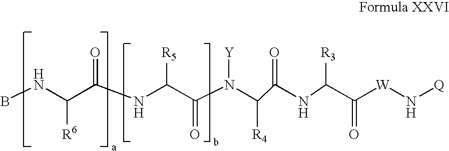
or a pharmaceutically acceptable salt, solvate or ester thereof; wherein in Formula XXVI above
B is an acyl derivative of formula R₁₁, —C(O)— wherein R₁₁ is Cl-10 alkyl optionally substituted with carboxyl; or R₁₁ is C₆ or C₁₀ aryl or C₇₋₁₆ aralkyl optionally substituted with a C₁₋₆ alkyl;
a is 0 or 1;
R₆, when present, is carboxy(lower)alkyl;
b is 0 or 1;
R₅, when present, is C₁₋₆ alkyl, or carboxy(lower)alkyl;
Y is H or C₁₋₆ alkyl;
R₄ is C₁₋₁₀ alkyl; C₃₋₁₀ cycloalkyl;
R₃ is C1-10 alkyl; C₃₋₁₀ cycloalkyl;
W is a group of formula: 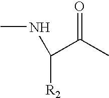
wherein R₂ is C₁₋₁₀ alkyl or C₃₋₇ cycloalkyl optionally substituted with carboxyl; C₆ or C₁₀ aryl; or C₇₋₁₆ aralkyl; or
W is a group of formula: 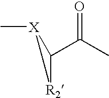
wherein X is CH or N; and
R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:
cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: O, S, and N; or
R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: O, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂. OH, SH, halo, carboxyl or carboxy(lower)alkyl; C₆ or C₁₀ aryl, or heterocycle; said second ring optionally containing at least one heteroatom selected independently from the group consisting of: O, S, and N;
Q is a group of the formula: 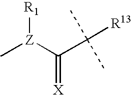
wherein Z is CH or N;
X is O or S;
R₁ is H, C₁₋₆ alkyl or C₁₋₆ alkenyl both optionally substituted with thio or halo;
and
when Z is CH, then R₁₃ is H; CF₃; CF₂CF₃; CH₂—R₁₄; CH(F)—R₁₄; CF₂—R₁₄; NR₁₄R₁₄′; S—R₁₄; or CO—NH—R₁₄ wherein R₁₄ and R₁₄′ are independently hydrogen, cyclic C₃₋₁₀ alkyl or acyclic C₁₋₁₀ alkyl or cyclic C₃₋₁₀ alkenyl or acyclic C₂₋₁₀ alkenyl, said alkyl or alkenyl optionally substituted with NH₂, OH, SH, halo or carboxyl; said alkyl or alkenyl optionally containing at least one heteroatom selected independently from the group consisting of: O, S, and N; or
R₁₄ and R₁₄′ are independently C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: O, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: O, S, and N;
or R₁₄ and R₁₄′ are independently C₁₋₄ alkyl which when joined together with N form a 3 to 6-membered nitrogen-containing ring which is optionally fused with a further C₃₋₇ cycloalkyl, C₆ or C₁₀ aryl or heterocycle;
with the proviso that when Z is CH, then R₁₃ is not an α-amino acid or an ester thereof;
when Z is N, then R₁₃ is H; carboxy; C₁₋₆ alkyl optionally substituted with carboxy; CH₂—R₁₄; CHR₁₄R₁₄′; CH(F)—R₁₄; O—R₁₄; NR₁₄R₁₄′ or S—R₁₄ wherein R₁₄ and R₁₄′ are as defined above; or
Q is a phosphonate group of the formula: 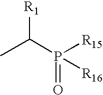
wherein R₁₅ and R₁₆ are independently C₆₋₂₀ aryloxy; and R₁ is as defined above.

2. A method for modulating the activity of Hepatitis C virus (HCV) protease in a subject, wherein the method comprises administering to a subject in need of such treatment a dosage formulation containing at least one HCV protease inhibitor in a pharmaceutically effective amount thereof, wherein the dosage formulation is capable of maintaining an average Cmin plasma concentration of the at least one HCV protease inhibitor at or above 10 ng/ml,
wherein the at least one HCV protease inhibitor is selected from the group consisting of compounds of Formulae I to XXVI below:
a. Formula I 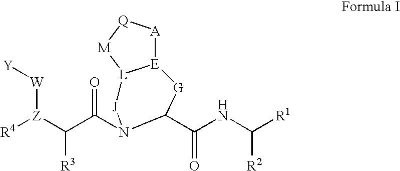
or a pharmaceutically acceptable salt, solvate or ester thereof,
wherein in Formula I above:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is H, OH, OR⁸, NR⁹R¹⁰, CF₃, C₂F₅, C₃F₇, CF₂R⁶, R⁶, or COR⁷ wherein R⁷ is H, OH, OR⁸, CHR⁹R¹⁰, or NR⁹R¹⁰, wherein R⁶, R⁸, and R¹⁰ are independently selected from the group consisting of H, alkyl, aryl, heteroalkyl, heteroaryl, cycloalkyl, cycloalkyl, arylalkyl, heteroarylalkyl, [CH(R^{1′})]ₚCOOR¹¹, [CH(R^{1′})]ₚCONR¹²R¹³, [CH(R^{1′})]ₚSO₂R¹¹, [CH(R^{1′})]ₚCOR¹¹, [CH(R^{1′})]ₚCH(OH)R¹¹, CH(R^{1′})CONHCH(R²)COOR¹¹, CH(R^{1′})CONHCH(R^{2′})CONR¹²R¹³, CH(R^{1′})CONHCH(R²)R′, CH(R^{1′})CONHCH(R^{2′})CONHCH(R^{3′})COOR¹¹, CH(R^{1′})CONHCH(R^{2′})CONHCH(R^{3′})CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹ and CH(R^{1′})CONHCH(R^{2′})CONHCH(R^{3′})CONHCH(R^{4′})CONHCH(R^{5′}) CONR¹²R¹³, wherein R^{1′}, R^{2′}, R^{3′}, R^{4′}, R^{5′}, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;
Z is selected from O, N, CH or CR;
W maybe present or absent, and if W is present, W is selected from C═O, C═S, C(═N—CN), or SO₂;
Q maybe present or absent, and when Q is present, Q is CH, N, P, (CH₂)ₚ, (CHR)ₚ, (CRR′)ₚ, O, NR, S, or SO₂; and when Q is absent, M may be present or absent;
when Q and M are absent, A is directly linked to L;
A is O, CH₂, (CHR)ₚ, (CHR—CHR′)ₚ, (CRR′)ₚ, NR, S, SO₂ or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;
L may be present or absent, and when L is present, L is CH, CR, O, S or NR; and when L is absent, then M may be present or absent; and if M is present with L being absent, then M is directly and independently linked to E, and J is directly and independently linked to E;
M may be present or absent, and when M is present, M is O, NR, S, SO₂, (CH₂)ₚ, (CHR)ₚ(CHR—CHR′)ₚ, or (CRR′)ₚ;
p is a number from 0 to 6; and
R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen;
(cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring
b. Formula II 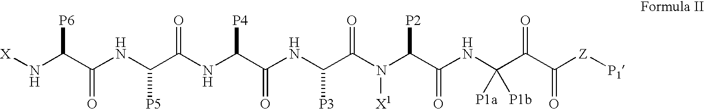
or a pharmaceutically acceptable salt, solvate or ester thereof;
wherein in Formula II above:
Z is O, NH or NR¹²;
X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylalkylcarbonyl, arylcarbonyl, heteroarylcarbonyl, alkoxycarbonyl, heterocyclyloxycarbonyl, aryloxycarbonyl, heteroaryloxycarbonyl, alkyaminocarbonyl, heterocyclylaminocarbonyl, arylaminocarbonyl, or heteroarylaminocarbonyl moiety, with the proviso that X may be additionally optionally substituted with R¹² or R¹³;
X¹ is H; C₁-C₄ straight chain alkyl; C₁-C₄ branched alkyl or; CH₂-aryl (substituted or unsubstituted);
R¹² is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R¹² may be additionally optionally substituted with R¹³.
R¹³ is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³. P1 a, P1b, P2, P3, P4, P5, and P6 are independently:
H; C1-C10 straight or branched chain alkyl; C2-C10 straight or branched chain alkenyl;
C3-C8 cycloalkyl, C3-C8 heterocyclic; (cycloalkyl)alkyl or (heterocyclyl)alkyl, wherein said cycloalkyl is made up of 3 to 8 carbon atoms, and zero to 6 oxygen, nitrogen, sulfur, or phosphorus atoms, and said alkyl is of 1 to 6 carbon atoms;
aryl, heteroaryl, arylalkyl, or heteroarylalkyl, wherein said alkyl is of 1 to 6 carbon atoms;
wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and
P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R¹³;
h. Formula III 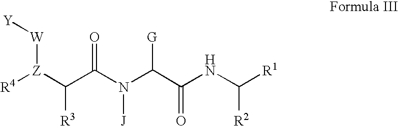
or a pharmaceutically acceptable salt, solvate or ester thereof;
wherein in Formula III above:
G, J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe additionally optionally substituted with X¹¹ or X¹²;
X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl, CH(R¹′)COOR¹¹, CH(R¹′)CONR¹²R¹³, CH(R¹′)CONHCH(R²′)COOR¹¹, CH(R¹′)CONHCH(R²′)CONR¹²R¹³, CH(R¹′)CONHCH(R²′)R′, CH(R¹′)CONHCH(R²′)CONHCH(R³′)COOR¹¹, CH(R¹′)CONHCH(R²′)CONHCH(R³′)CONR¹²R¹³, CH(R¹′)CONHCH(R²′)CONHCH(R³′)CONHCH(R⁴′)COOR¹¹, CH(R¹′)CONHCH(R²′)CONHCH(R³′)CONHCH(R⁴′)CONR¹²R¹³, CH(R¹′)CONHCH(R²′)CONHCH(R³′)CONHCH(R⁴′)CONHCH(R⁵′)COO R¹¹, and CH(R¹′)CONHCH(R²′)CONHCH(R³′)CONHCH(R⁴′)CONHCH(R⁵′)CONR¹²R¹³, wherein R¹′, R²′, R³′, R⁴′, R⁵′, R¹¹, R¹², R¹³, and R′ may be the s or different and are independently selected from a group consisting of H, alkyl, aryl, heteroalkyl, heteroaryl, cycloalkyl, alkyl-aryl, alkyl-heteroaryl, aryl-alkyl and heteroaralkyl;
Z is selected from O, N, or CH;
W maybe present or absent, and if W is present, W is selected from C═O, C═S, or SO₂; and
R, R′, R², R³ and R⁴ are independently selected from the group consisting of H; C1-C10 alkyl; C2-C10 alkenyl; C3-C8 cycloalkyl; C3-C8 heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate;
i. Formula IV 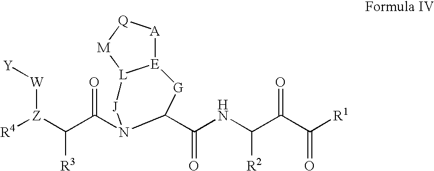
or a pharmaceutically acceptable salt, solvate or ester thereof;
wherein in Formula IV above:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is selected from the following structures: 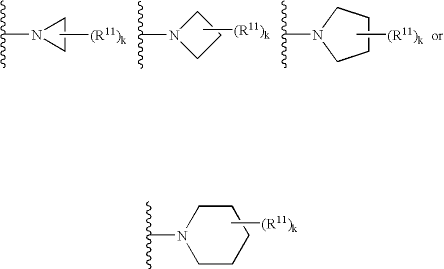
wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino,arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹≠H) maybe optionally substituted with X¹¹ or X¹²;
Z is selected from O, N, CH or CR;
W may be present or absent, and if W is present, W is selected from C═O, C═S, C(═N—CN), or S(O₂);
Q may be present or absent, and when Q is present, Q is CH, N, P, (CH₂)ₚ, (CHR)ₚ, (CRR′)ₚ, O, N(R), S, or S(O₂); and when Q is absent, M may be present or absent;
when Q and M are absent, A is directly linked to L;
A is O, CH₂, (CHR)ₚ, (CHR—CHR′)ₚ, (CRR′)ₚ, N(R), S, S(O₂) or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J may be present or absent, and when J is present, J is (CH₂)ₚ, (CHR)ₚ, or (CRR′)ₚ, S(O₂), NH, N(R) or O; and when J is absent, G is present and E is directly linked to N shown in Formula I as linked to J;
L may be present or absent, and when L is present, L is CH, C(R), O, S or N(R); and
when L is absent, then M may be present or absent; and if M is present with L being absent, then M is directly and independently linked to E, and J is directly and independently linked to E;
M may be present or absent, and when M is present, M is O, N(R), S, S(O₂), (CH₂)ₚ, (CHR)ₚ (CHR—CHR′)ₚ, or (CRR′)ₚ;
p is a number from 0 to 6; and
R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered cyclic ring structure or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring;
j. Formula V 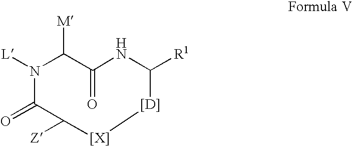
or a pharmaceutically acceptable salt, solvate or ester thereof,
wherein in Formula V above:
(1) R¹ is —C(O)R⁵ or —B(OR)₂;
(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;
(3) R⁷ is H, —OH, —OR⁸,or —CHR⁹R¹⁰;
(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R^{1′})CH(R^{1′})C(O)OR¹¹,[CH(R^{1′})]ₚC(O)OR¹¹,—[CH(R^{1′})]ₚC(O)NR¹²R¹³,—[CH(R^{1′})]ₚ S(O₂)R¹¹, —[CH(R^{1′})]ₚC(O)R¹¹, —[CH(R^{1′})]ₚS(O₂)NR¹²R¹³, CH(R^{1′})C(O)N(H)CH(R²′)(R′), CH(R^{1′})CH(R^{1′})C(O)NR¹²R¹³, —CH(R^{1′})CH(R^{1′})S(O₂)R¹¹, —CH(R^{1′})CH(R^{1′})S(O₂)NR¹²R¹³, —CH(R^{1′})CH(R^{1′})C(O)R¹¹, —[CH(R^{1′})]ₚCH(OH)R¹¹,—CH(R^{1′})C(O)N(H)CH(R^{2′})C(O)OR¹¹, C(O)N(H)CH(R^{2′})C(O)OR¹¹,—C(O)N(H)CH(R^{2′})C(O)R¹¹, CH(R^{1′})C(O)N(H)CH(R^{2′})C(O) NR¹²R¹³,—CH(R^{1′})C(O)N(H)CH(R^{2′})R′, CH(R^{1′})C(O)N(H)CH(R^{2′})C(O)N(H)CH(R^{3′})C(O)OR¹¹, CH(R^{1′})C(O)N(H)CH(R^{2′})C(O)CH(R^{3′})NR¹²R¹³, CH(R^{1′})C(O)N(H)CH(R^{2′})C(O)N(H)CH(R^{3′})C(O)NR¹²R¹³, CH(R^{1′})C(O)N(H)CH(R^{2′})C(O)N(H)CH(R^{3′})C(O)N(H)CH(R^{4′})C(O)OR¹¹, CH(R^{1′})C(O)N(H)CH(R^{2′})C(O)N(H)CH(R^{3′})C(O)N(H)CH(R^{4′})C(O)NR¹²R¹³, CH(R^{1′})C(O)N(H)CH(R²)C(O)N(H)CH(R^{3′})C(O)N(H)CH(R^{4′})C(O)N(H)CH(R^{5′})C(O)OR¹¹, and CH(R^{1′})C(O)N(H)CH(R^{2′})C(O)N(H)CH(R^{3′})C(O)N(H)CH(R^{4′})C(O)N(H)CH(R^{5′})C(O)NR¹²R¹³;
wherein R¹, R^{2′}, R^{3′}, R^{4′}, R^{5′}, R¹¹, R¹² and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl; or
R¹² and R¹³ are linked together wherein the combination is cycloalkyl, heterocycloalkyl, ary or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain;
or L′ and M′ are linked together to form a ring structure wherein the portion of structural Formula 1 represented by 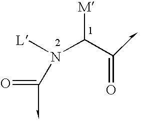
is represented by structural Formula 2: 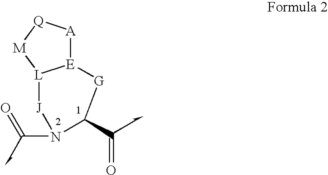
wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;
L is present or absent, and when L is present, L is C(H) or C(R); when L is absent, M is present or absent; if M is present with L being absent, then M is directly and independently linked to E, and J is directly and independently linked to E;
G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;
Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent being selected from —OR, —CH(R)(R′), S(O)₀₋₂R or —NRR′ or (iii) absent; when both Q and M are absent, A is either directly linked to L, or A is an independent substituent on E, said independent substituent bing selected from —OR, —CH(R)(R′), S(O)₀₋₂R or —N RR′ or A is absent;
A is present or absent and if present A is 0, O(R), (CH₂)p, (CHR)P , (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, O, OR, N(R), S, S(O₂), (CH₂)ₚ, (CHR)ₚ (CHR—CHR′)ₚ, or (CRR′)ₚ; or M is linked to A to form an alicyclic, aliphatic or heteroalicyclic bridge;
(8) Z′ is represented by the structural Formula 3: 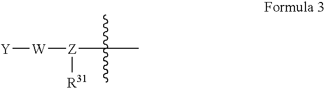
wherein in Formula 3, Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹¹ is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
Z is O, N, C(H) or C(R);
R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
W may be present or absent, and if W is present, W is C(═O), C(═S), C(═N—CN), or S(O₂);
(9) X is represented by structural Formula 4: 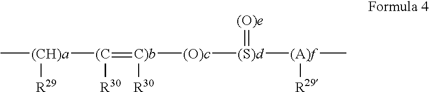
wherein in Formula 4, a is 2, 3, 4, 5, 6, 7, 8 or 9;
b, c, d, e and f are 0, 1, 2, 3, 4 or 5;
A is C, N, S or O;
R²⁹ and R^{29′} are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R²⁹ and R^{29′} are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;
(10) D is represented by structural Formula 5: 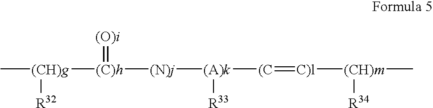
wherein in Formula 5, R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R³² and R³⁴ are linked together such that the combination forms a portion of a cycloalkyl group;
g is 1, 2, 3, 4, 5, 6, 7, 8 or 9;
h, i, j, k, l and m are 0, 1, 2, 3, 4 or 5; and
A is C, N, S or O,
(11) provided that when structural Formula 2: 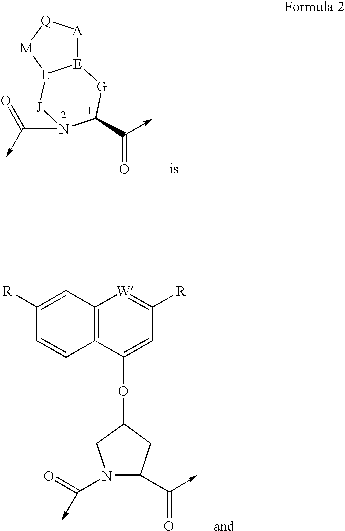
W′ is CH or N, both the following conditional exclusions (i) and (ii) apply:
conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆ alkyl or C₃₋₆ cycloalkyl; and
conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈ alkyl, C₃₋₆ cycloalkyl, C_{6 to 10} aryl or C₇₋₁₆ aralkyl;
k. Formula VI 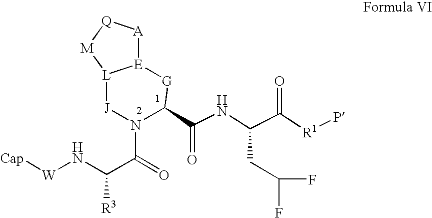
or a pharmaceutically acceptable salt, solvate or ester of said compound, wherein in Formula VI above:
Cap and P′ are independently H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino can be unsubstituted or optionally independently substituted with one or two substituents which can be the same or different and are independently selected from X¹ and X²;
X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;
X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;
W may be present or absent, and when W is present W is C(═O), C(═S), C(═NH), C(═N—OH), C(═N—CN), S(O) or S(O₂);
Q maybe present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R′), S(O)₀₋₂R or —NRR′; when both Q and M are absent, A is either directly linked to L, or A is an independent substituent on E, selected from —OR, CH(R)(R′), —S(O)₀₋₂R or —NRR′;
A is present or absent and if present A is —O—, —O(R) CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either Q and E are connected by a bond or Q is an independent substituent on M;
E is present or absent and if present E is CH, N, C(R);
G may be present or absent, and when G is present, G is (CH₂)ₚ, (CHR)ₚ, or (CRR′)ₚ; when G is absent, J is present and E is directly connected to the carbon atom marked position 1;
J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
L may be present or absent, and when L is present, L is CH, N, or CR; when L is absent, M is present or absent; if M is present with L being absent, then M is directly and independently linked to E, and J is directly and independently linked to E;
M may be present or absent, and when M is present, M is O, N(R), S, S(O₂), (CH₂)ₚ, (CHR)ₚ, (CHR—CHR′)ₚ, or (CRR′)ₚ;
p is a number from 0 to 6;
R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;
R and R′ in (CRR′) can be linked together such that the combination forms a cycloalkyl or heterocyclyl moiety; and
R¹ is N(R) or O;
I. Formula VII 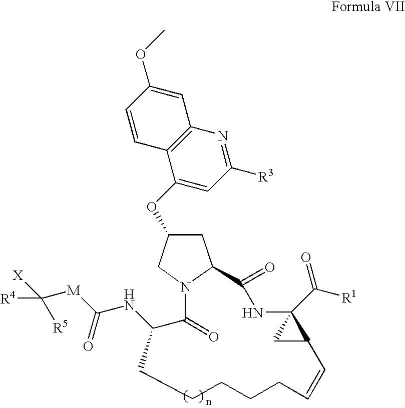
or a pharmaceutically acceptable salt, solvate or ester thereof, wherein in Formula VII above:
M is O, N(H), or CH₂;
n is 0-4;
R¹ is —OR⁶, —NR⁶R⁷ or 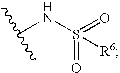
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7- membered ring such that the moiety 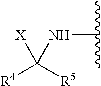
is represented by 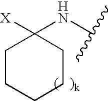
where k is 0 to 2;
X is selected from the group consisting of: 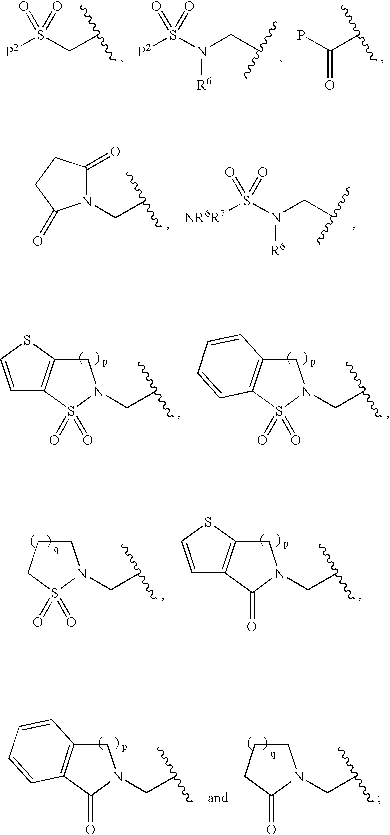
where p is 1 to 2, q is 1-3 and p² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 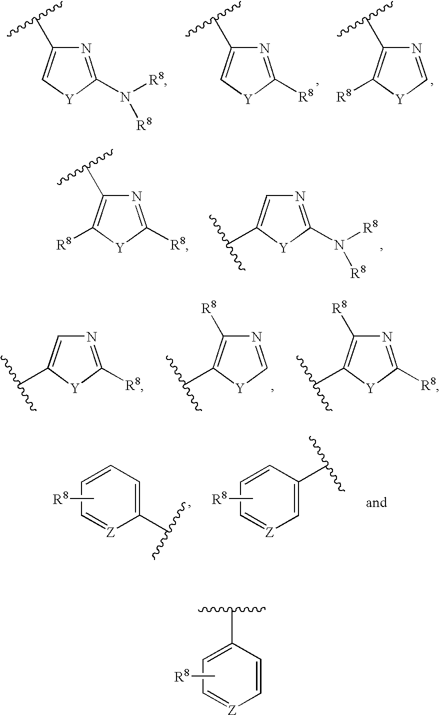
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
h. Formula VIII 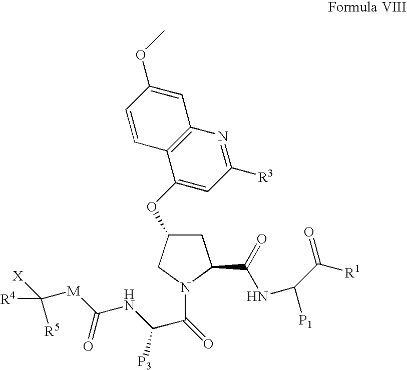
or a pharmaceutically acceptable salt, solvate or ester thereof, wherein in Formula VIII above,
M is O, N(H), or CH₂;
R¹ is —OR⁶, —NR⁶R⁷ or 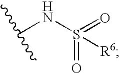
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
P₁ is selected from the group consisting of alkyl, alkenyl, alkynyl, cycloalkyl haloalkyl;
P₃ is selected from the group consisting of alkyl, cycloalkyl, aryl and cycloalkyl fused with aryl;
R⁴ and R⁵ can be the same or different, each being independently selected from the group consisting of H, alkyl, aryl and cycloalkyl; or alternatively R⁴ and R⁵ together form part of a cyclic 5- to 7- membered ring such that the moiety 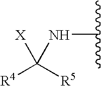
is represented by 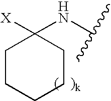
where k is 0 to 2;
X is selected from the group consisting of: 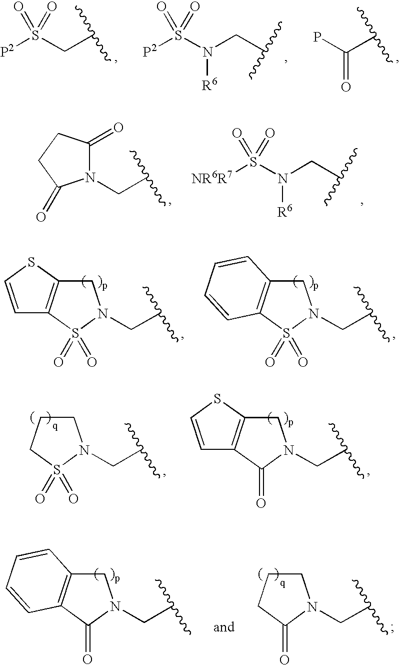
where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 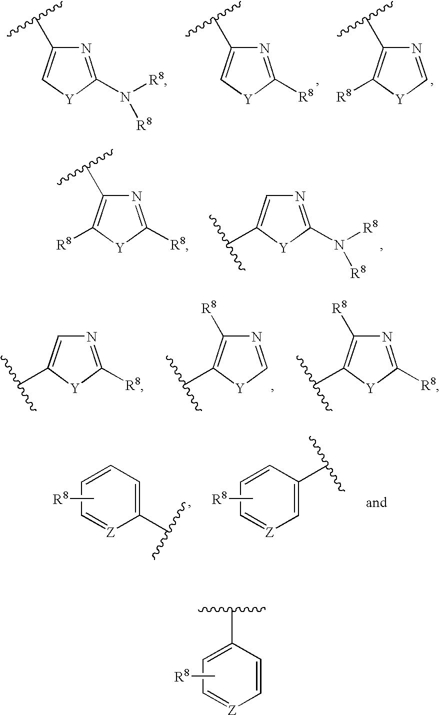
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
i. Formula IX 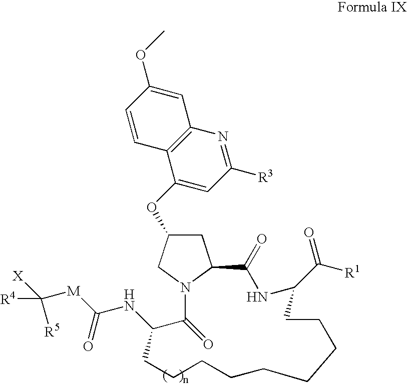
or a pharmaceutically acceptable salt, solvate or ester thereof, wherein in Formula IX above,
M is O, N(H), or CH₂;
n is 0-4;
R¹ is —R⁶, —NR⁶R⁷ or 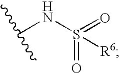
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7- membered ring such that the moiety 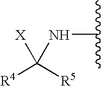
is represented by 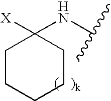
where k is 0 to 2;
X is selected from the group consisting of: 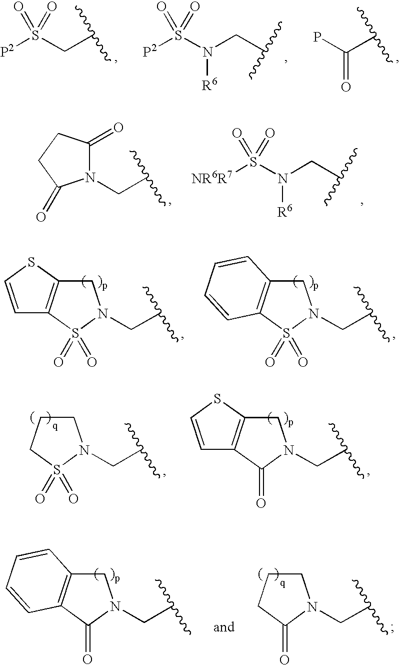
where p is 1 to 2, q is 1 to 3 and P² iS alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 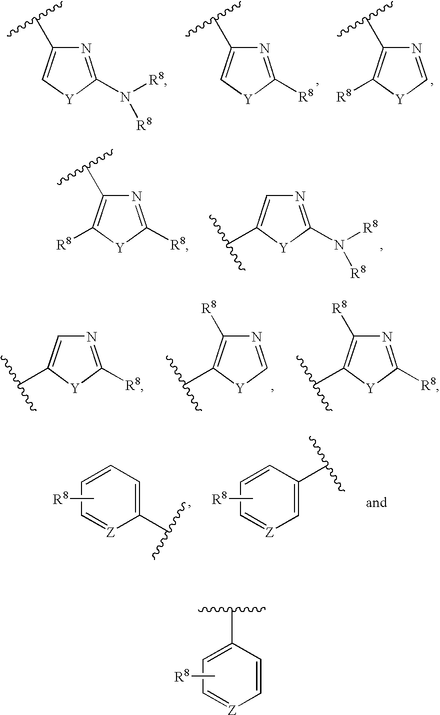
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
r. Formula X 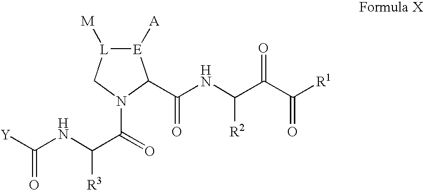
or a pharmaceutically acceptable salt, solvate or ester thereof;
wherein in Formula X above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 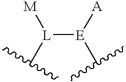
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl )alkyl-, (heterocyclyl )alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 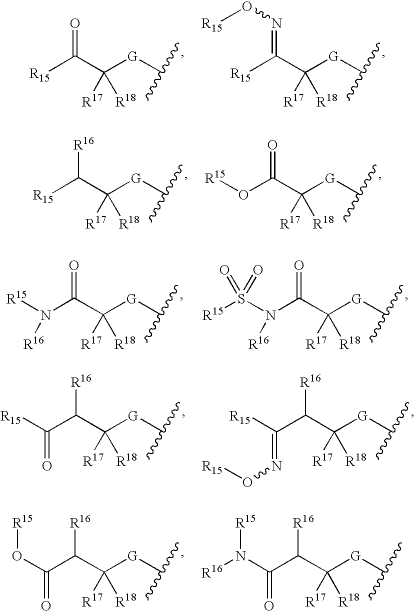
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
s. Formula XI 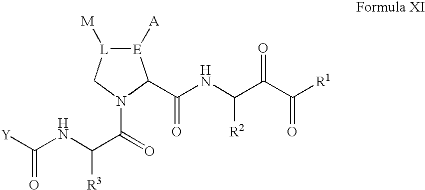
or a pharmaceutically acceptable salt, solvate or ester thereof; wherein in Formula XI above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, NR⁹R¹⁰, SR, SO₂R, and halo; or A and M are connected to each other (in other words, A-E-L-M taken together) such that the moiety: 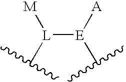
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;
Y is selected from the following moieties: 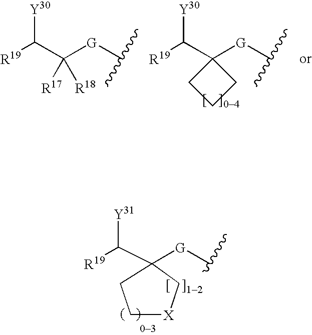
wherein Y³⁰ and Y³¹ are selected from 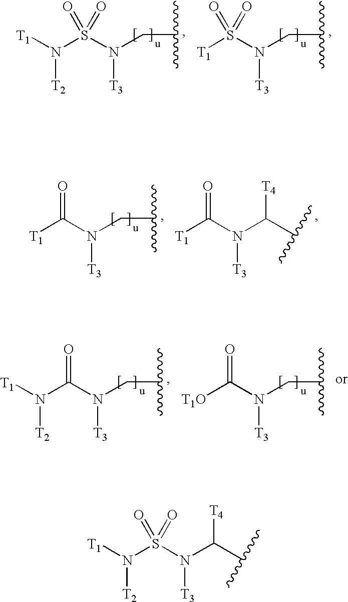
where u is a number 0-6;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, T₃ and T₄ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
t. Formula XII 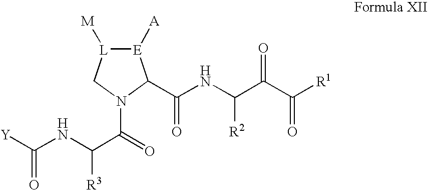
or a pharmaceutically acceptable salt, solvate or ester thereof; wherein in Formula XII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 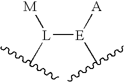
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 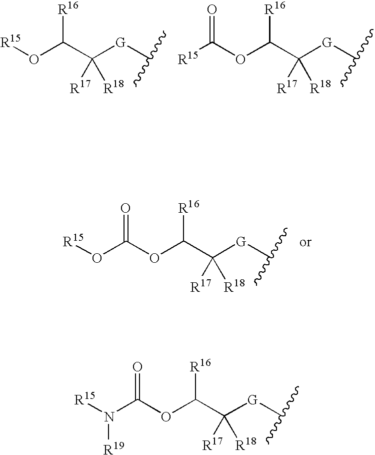
wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro;
u. Formula XIII 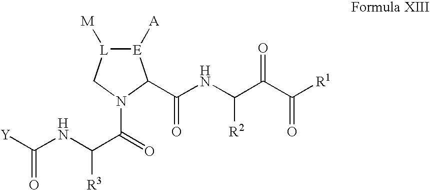
or a pharmaceutically acceptable salt, solvate or ester thereof; wherein in Formula XIII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other (in other words, A-E-L-M taken together) such that the moiety: 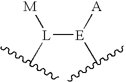
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 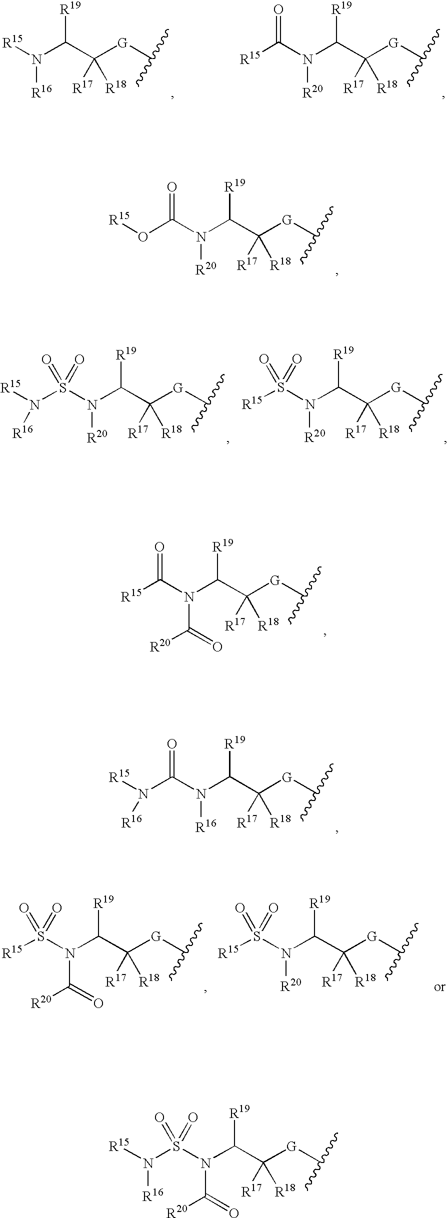
wherein G is NH or O, and R¹⁵, R¹⁶, R¹⁷, R¹⁸ R¹⁹ and R²⁰ can be the same or different, each being independently selected from the group consisting of H, C-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
v. Formula XIV 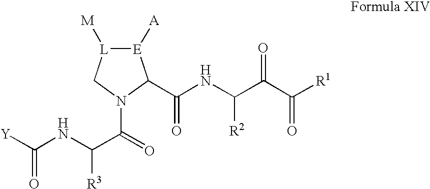
or a pharmaceutically acceptable salt, solvate or ester thereof; wherein in Formula XIV above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo;
or A and M are connected to each other such that the moiety: 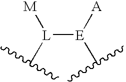
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;
and Y is selected from the following moieties: 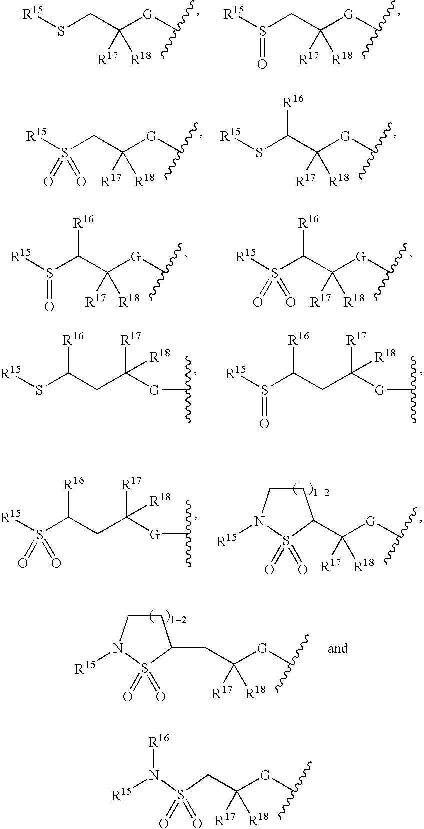
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro;
w. Formula XV 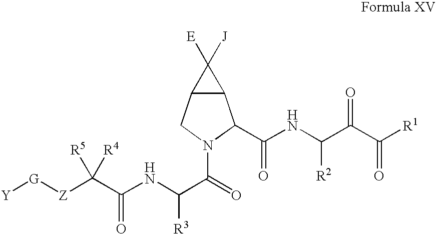
or a pharmaceutically acceptable salt, solvate or ester thereof; wherein in Formula XV above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, aryl-, heteroalkyl-, heteroaryl-, cycloalkyl-, cycloalkyl-, arylalkyl-, and heteroarylalkyl;
E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;
Z is N(H), N(R), or O, with the proviso that when Z is O, G is present or absent and if G is present with Z being O, then G is C(═O);
G maybe present or absent, and if G is present, G is C(═O) or S(O₂), and when G is absent, Z is directly connected to Y;
Y is selected from the group consisting of: 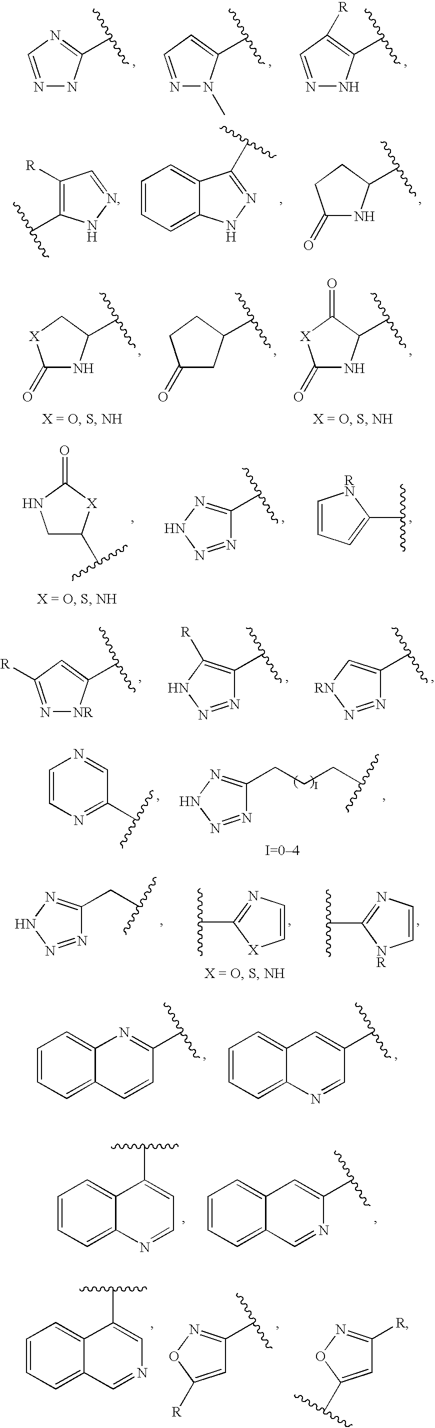 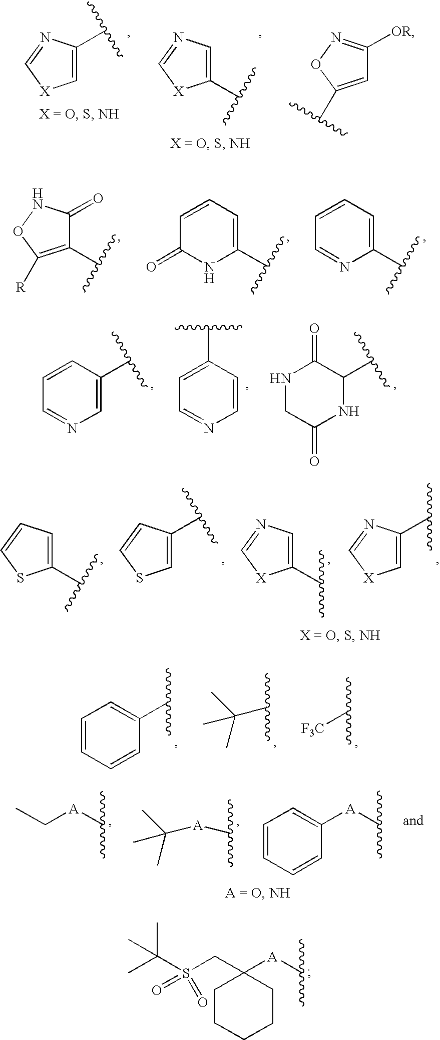
R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;
wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
x. Formula XVI 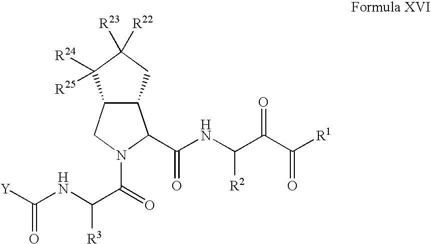
or a pharmaceutically acceptable salt, solvate or ester thereof; wherein in Formula XVI above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 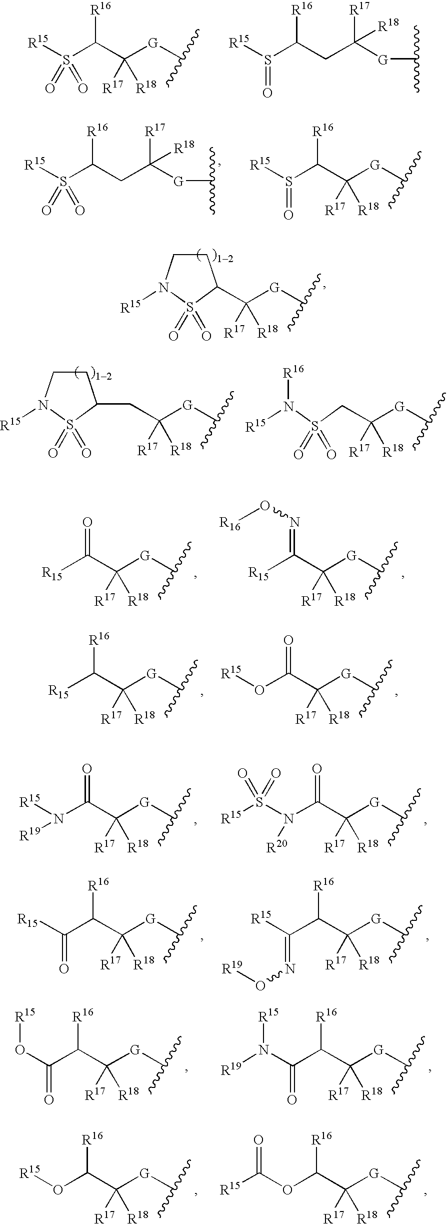 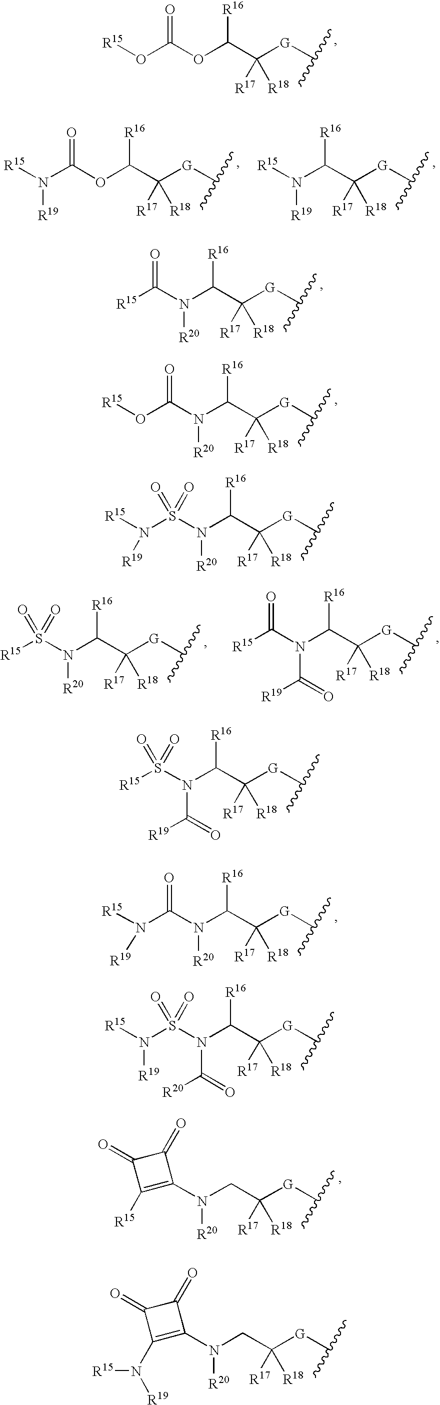
wherein G is NH or O; and R¹⁵, R¹⁶, R¹⁷, R¹⁸, R¹⁹, R²⁰, R²¹, R²², R²³, R²⁴, and R²⁵ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
y. Formula XVII 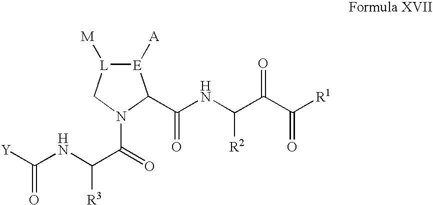
or a pharmaceutically acceptable salt, solvate or ester thereof; wherein in Formula XVII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 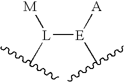
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
Y is selected from the following moieties: 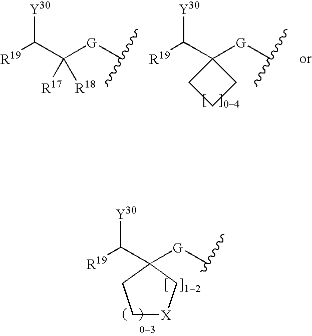
wherein Y³⁰ is selected from 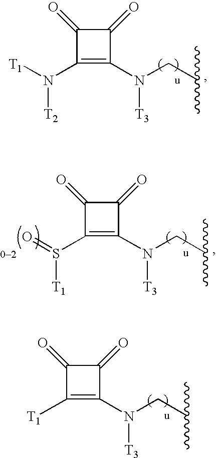
where u is a number 0-1;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
r. Formula XVIII 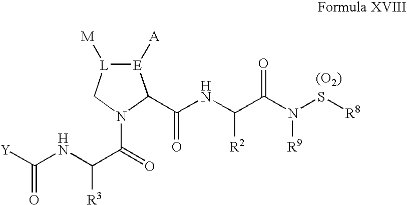
or a pharmaceutically acceptable salt, solvate or ester thereof, wherein in Formula XVIII above:
R⁸ is selected from the group consisting of alkyl-, aryl-, heteroalkyl-, heteroaryl-, cycloalkyl-, heterocyclyl-, arylalkyl-, heteroarylalkyl- , and heterocyclylalkyl;
R⁹ is selected from the group consisting of H, alkyl, alkenyl, alkynyl, aryl and cycloalkyl;
A and M can be the same or different, each being independently selected from R, OR, N(H)R, N(RR′), SR, S(O₂)R, and halo; or A and M are connected to each other (in other words, A-E-L-M taken together) such that the moiety: 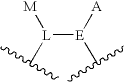
shown above in Formula I forms either a three, four, five, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl;
R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;
Y is selected from the following moieties: 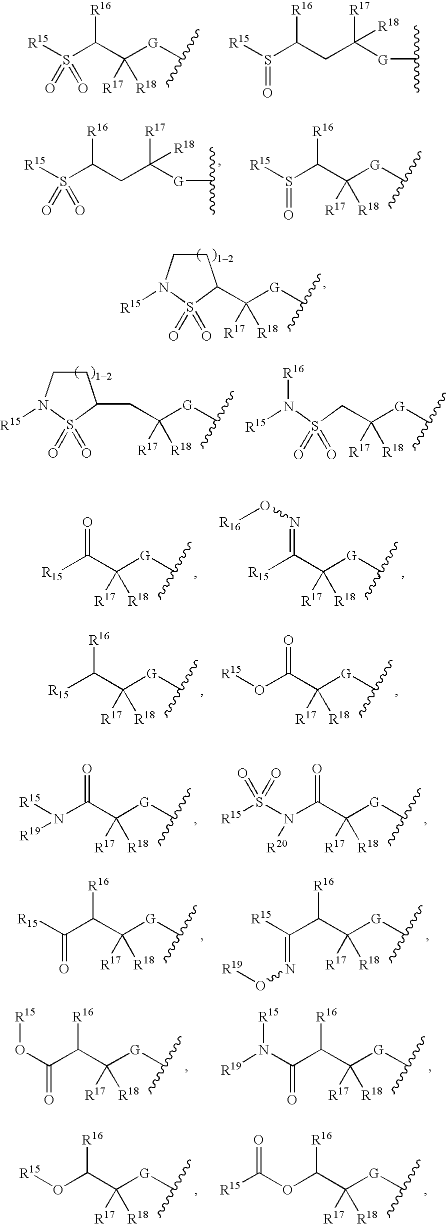 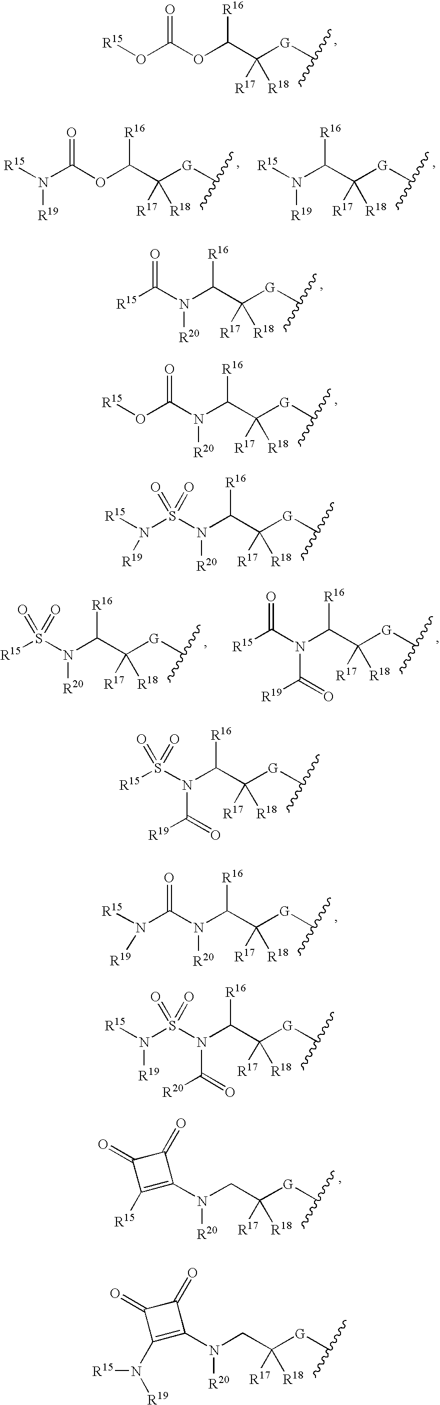
wherein G is NH or O; and R¹⁵, R¹⁶, R¹⁷, R¹⁸, R¹⁹ and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
s. Formula XIX 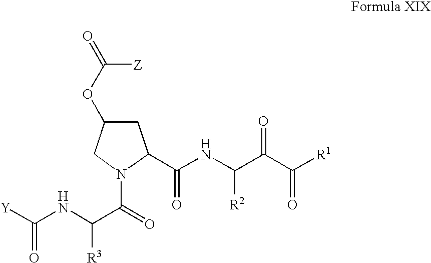
wherein in Formula XIX above:
Z is selected from the group consisting of a heterocyclyl moiety,
N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N(heteroaryl)₂;
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 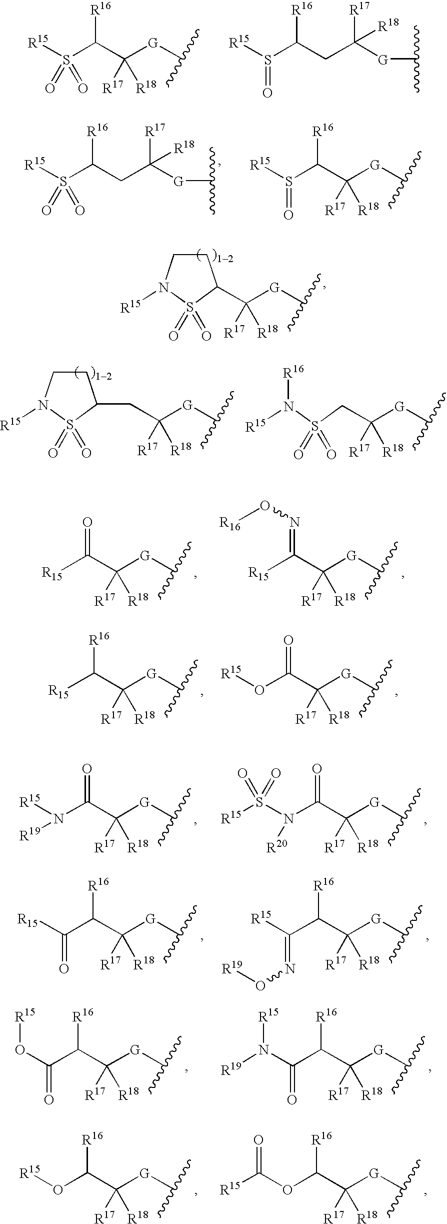 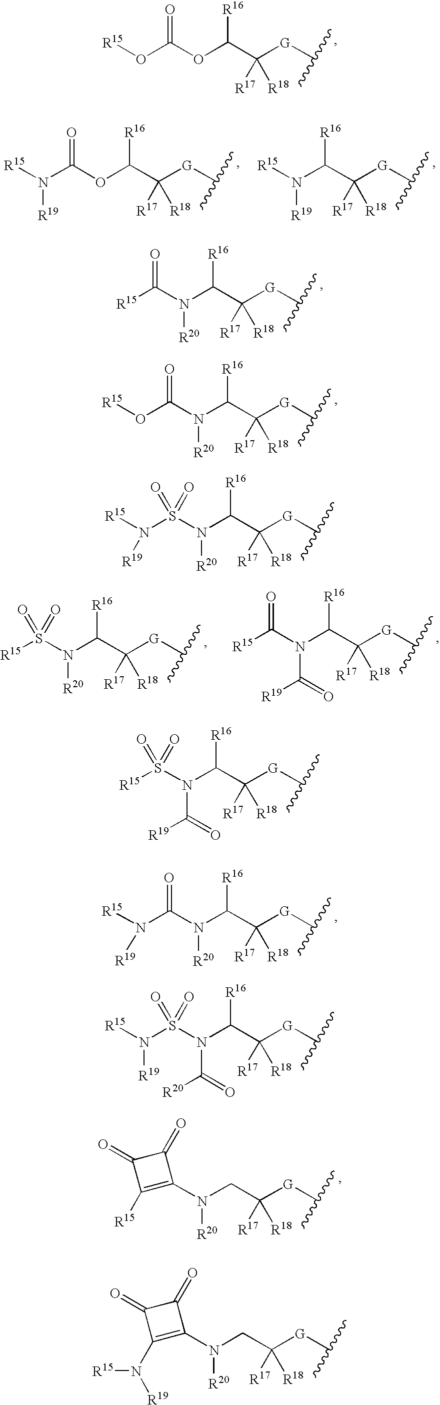
wherein G is NH or O; and R¹⁵, R¹⁶, R¹⁷, R¹⁸, R¹⁹, R²⁰ and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
u. Formula XX 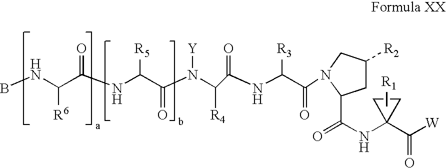
or a pharmaceutically acceptable salt, solvate or ester thereof; wherein in Formula XX above:
a is 0 or 1; b is 0 or 1; Y is H or C₁₋₆ alkyl;
B is H, an acyl derivative of formula R₇—C(O)— or a sulfonyl of formula R₇—SO2 wherein
R7 is (i) C₁₋₁₀ alkyl optionally substituted with carboxyl, C₁₋₆ alkanoyloxy or C₁₋₆ alkoxy;
(ii) C₃₋₇ cycloalkyl optionally substituted with carboxyl, (C₁₋₆ alkoxy)carbonyl or phenylmethoxycarbonyl;
(iii) C₆ or C₁₀ aryl or C₇₋₁₆ aralkyl optionally substituted with C₁₋₆ alkyl, hydroxy, or amino optionally substituted with C₁₋₆ alkyl; or
(iv) Het optionally substituted with C₁₋₆ alkyl, hydroxy, amino optionally substituted with C₁₋₆ alkyl, or amido optionally substituted with C₁₋₆ alkyl;
R₆, when present, is C₁₋₆ alkyl substituted with carboxyl;
R₅, when present, is C₁₋₆ alkyl optionally substituted with carboxyl;
R₄ is C₁₋₁₀ alkyl, C₃₋₇ cycloalkyl or C₄₋₁₀ (alkylcycloalkyl);
R₃ is C₁₋₁₀ alkyl, C₃₋₇ cycloalkyl or C₄₋₁₀ (alkylcycloalkyl);
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri-substituted with R₂₁,
or R₂₀ is Het or (lower alkyl)-Het optionally mono-, di- or tri-substituted with R₂₁, wherein each R₂₁ is independently C₁₋₆ alkyl; C₁₋₆alkoxy; amino optionally mono- or di-substituted with C₁₋₆ alkyl; sulfonyl; NO₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;
wherein R₂₂ is C₁₋₆alkyl; C₁₋₆ alkoxy; amino optionally mono- or di-substituted with C₁₋₆ alkyl; sulfonyl; NO₂; OH; SH; halo; haloalkyl; carboxyl; amide or (lower alkyl)amide;
R₁ is C₁₋₆ alkyl or C₂₋₆ alkenyl optionally substituted with halogen; and
W is hydroxy or a N-substituted amino;
u. Formula XXI: 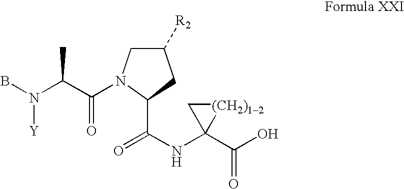
or a pharmaceutically acceptable salt, solvate or ester thereof; wherein in Formula XXI above:
B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl of formula R₄—O—C(O)—; an amide of formula R₄—N(R₅)—C(O)—; a thioamide of formula R₄—N(R₅)—C(S)—; or a sulfonyl of formula R₄—SO2 wherein
R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;
(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;
(iii) amino optionally mono- or di-substituted with C₁₋₆ alkyl; amido; or (lower alkyl)amide;
(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or
(v) Het or (lower alkyl)-Het, both optionally substituted with C₁₋₆ alkyl, hydroxy, amido, (lower alkyl) amide, or amino optionally mono- or di-substituted with C₁₋₆ alkyl;
R₅ is H or C₁₋₆ alkyl;
with the proviso that when R₄ is an amide or a thioamide, R₄ is not (ii) a cycloalkoxy;
Y is H or C₁₋₆ alkyl;
R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,
or R₂₀ is Het or (lower alkyl)-Het, both optionally mono-, di- or tri-substituted with R₂₁,
wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;
wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;
R₁ is H; C₁₋₆ alkyl, C₃₋₇ cycloalkyl, C₂₋₆ alkenyl, or C₂₋₆ alkynyl, all optionally substituted with halogen;
v. Formula XXII: 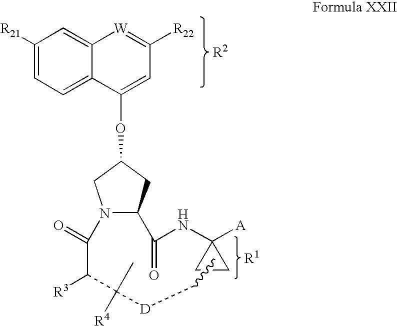
or a pharmaceutically acceptable salt, solvate or ester thereof; wherein in Formula XXII above:
W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂, wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵, wherein each R²⁵ is independently: H, C₁₋₆ alkyl or C₃₋₆ cycloalkyl;
or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹, wherein
R⁴¹ is H, C₁₋₆ alkyl, C₃₋₆ cycloalkyl or —C(O)—R⁴²,
wherein R⁴² is C₁₋₆ alkyl, C₃₋₆ cycloalkyl or C_{6 or 10} aryl;
R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C 1-6 thioalkyl, and
A is an amide of formula —C(O)—NH—R⁵, wherein R⁵ is selected from the group consisting of: C₁₋₈ alkyl, C₃₋₆ cycloalkyl, C_{6 or 10} aryl and C₇₋₁₆ aralkyl;
or A is a carboxylic acid;
w. Formula XXIII: 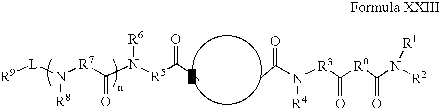
a pharmaceutically acceptable salt, solvate or ester thereof; wherein in Formula XXIII above:
R⁰ is a bond or difluoromethylene;
R¹ is hydrogen, optionally substituted aliphatic group, optionally substituted cyclic group or optionally substituted aromatic group;
R² and R⁹ are each independently optionally substituted aliphatic group, optionally substituted cyclic group or optionally substituted aromatic group;
R3, R5 and R7 are each independently:
optionally substituted (1,1- or 1,2-)cycloalkylene; or
optionally substituted (1,1- or 1,2-) heterocyclylene; or
methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or;
R4, R 6, R8 and R¹⁰ are each independently hydrogen or optionally substituted aliphatic group; 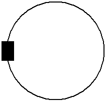
is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-(N(R⁸)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰OC(O)—, —S(O)₂—, or —NR¹⁰S(O)₂—; and n is 0 or 1, provided
when 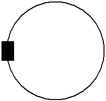
is substituted 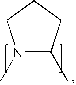
then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴ is optionally substituted aliphatic;
x. Formula XXIV: 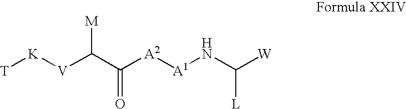
or a pharmaceutically acceptable salt, solvate or ester thereof; wherein in Formula XXIV above:
W is: 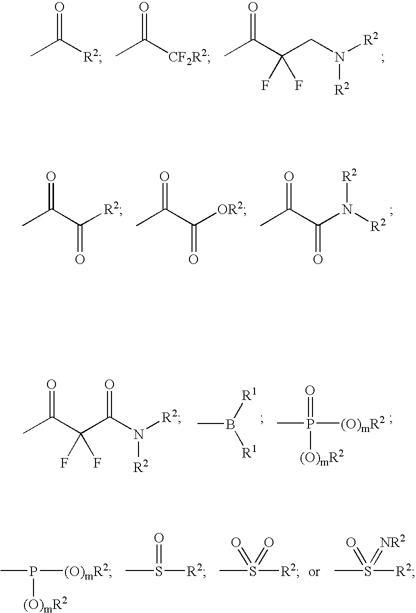
m is 0 or 1;
each R¹ is hydroxy, alkoxy, or aryloxy, or each R¹ is an oxygen atom and together with the boron, to which they are each bound, form a 5-7 membered ring, wherein the ring atoms are carbon, nitrogen, or oxygen;
each R² is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl, or two R² groups, which are bound to the same nitrogen atom, form together with that nitrogen atom, a 5-7 membered monocyclic heterocyclic ring system; wherein any R² carbon atom is optionally substituted with J;
J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;
J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoaikyl, halo, cyano, nitro, formyl, sulfonyl, or sulfonamido;
L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;
A¹ is a bond;
R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;
X is a bond, —C(H)(R7)-, —O—, —S—, or —N(R8)-;
R⁷ is hydrogen, alkyl, alkenyl, aryl, aralkyl, heterocyclyl, heterocyclylalkyl, heteroaryl, or heteroaralkyl, and is optionally substititued with 1-3 J groups;
R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R⁴, —SO₂R⁴, or carboxamido, and is optionally substititued with 1-3 J groups; or R⁸ and Z, together with the atoms to which they are bound, form a nitrogen containing mono- or bicyclic ring system optionally substituted with 1-3 J groups;
R¹⁴ is alkyl, aryl, aralkyl, heterocyclyl, heterocyclyalkyl, heteroaryl, or heteroaralkyl;
Y is a bond, —CH₂—, —C(O)—, —C(O)C(O)—, —S(O)—, —S(O)₂—, or —S(O)(NR⁷)—, wherein R⁷ is as defined above;
Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;
A² is a bond or 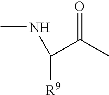
R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;
V is a bond, —CH₂—, —C(H)(R¹¹)—, —O—, —S—, or —N(R¹¹)—;
R¹¹ is hydrogen or C₁₋₃ alkyl;
K is a bond, —O—, —S—, —C(O)—, —S(O)—, —S(O)₂—, or —S(O)(NR¹¹)—, wherein R¹¹ is as defined above;
T is —R¹², -alkyl-R¹², -alkenyl-R¹², -alkynyl-R¹², —OR¹², —N(R¹²)2, —C(O)R¹², —C(═NOalkyl)R¹², or 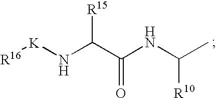
R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;
R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;
R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and
R¹⁶ is hydrogen, alkyl, aryl, heteroaryl, cycloalkyl, or heterocyclyl;
y. Formula XXV: 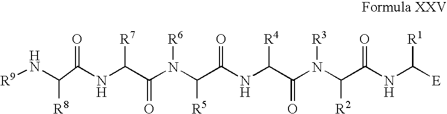
or a pharmaceutically acceptable salt, solvate or ester thereof;
wherein in Formula XVII above:
E represents CHO or B(OH)2;
R¹ represents lower alkyl, halo-lower alkyl, cyano-lower alkyl, lower alkylthio-lower alkyl, aryl-lower alkylthio-lower alkyl, aryl-lower alkyl, heteroaryllower alkyl, lower alkenyl or lower alkynyl;
R² represents lower alkyl, hydroxy-lower alkyl, carboxylower alkyl, aryl-lower alkyl, aminocarbonyl-lower alkyl or lower cycloalkyl-lower alkyl; and
R³ represents hydrogen or lower alkyl;
or R² and R³ together represent di- or trimethylene optionally substituted by hydroxy;
R4 represents lower alkyl, hydroxy-lower alkyl, lower cycloalkyl-lower alkyl, carboxy-lower alkyl, aryllower alkyl, lower alkylthio-lower alkyl, cyano-lower alkylthio-lower alkyl, aryl-lower alkylthio-lower alkyl, lower alkenyl, aryl or lower cycloalkyl;
R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;
R⁶ represents hydrogen or lower alkyl;
R⁷ represent lower alkyl, hydroxydower alkyl, carboxylower alkyl, aryl-iower alkyl, lower cycloalkyl-lower alkyl or lower cycloalkyl;
R⁸ represents lower alkyl, hydroxy-lower alkyl, carboxylower alkyl or aryl-lower alkyl; and
R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl; and
z. Formula XXVI: 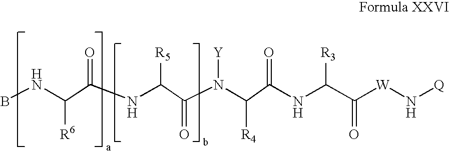
or a pharmaceutically acceptable salt, solvate or ester thereof; wherein in Formula XXVI above
B is an acyl derivative of formula R₁₁—C(O)— wherein R₁₁ is Cl-10 alkyl optionally substituted with carboxyl; or R₁₁ is C₆ or C₁₀ aryl or C₇₋₁₆ aralkyl optionally substituted with a C₁₋₆ alkyl;
a is 0 or 1;
R₆, when present, is carboxy(lower)alkyl;
b is 0 or 1;
R₅, when present, is C₁₋₆ alkyl, or carboxy(lower)alkyl;
Y is H or C₁₋₆ alkyl;
R₄ is C₁₋₁₀ alkyl; C₃₋₁₀ cycloalkyl;
R₃ is C1-10 alkyl; C₃₋₁₀ cycloalkyl;
W is a group of formula: 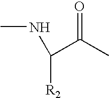
wherein R₂ is C₁₋₁₀ alkyl or C₃₋₇ cycloalkyl optionally substituted with carboxyl; C₆ or C₁₀ aryl; or C₇₋₁₆ aralkyl; or
W is a group of formula: 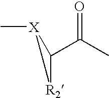
wherein X is CH or N; and
R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:
cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: O, S, and N; or
R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: O, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl; C₆ or C₁₀ aryl, or heterocycle; said second ring optionally containing at least one heteroatom selected independently from the group consisting of: O, S, and N;
Q is a group of the formula: 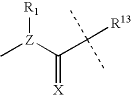
wherein Z is CH or N;
X is O or S;
R₁ is H, C₁₋₆ alkyl or C₁₋₆ alkenyl both optionally substituted with thio or halo; and
when Z is CH, then R₁₃ is H; CF₃; CF₂CF₃; CH₂—R₁₄; CH(F)—R₁₄; CF₂—R₁₄; NR₁₄R₁₄′; S—R₁₄; or CO—NH—R₁₄ wherein R₁₄ and R₁₄′ are independently hydrogen, cyclic C₃₋₁₀ alkyl or acyclic C₁₋₁₀ alkyl or cyclic C₃₋₁₀ alkenyl or acyclic C₂₋₁₀ alkenyl said alkyl or alkenyl optionally substituted with NH₂, OH, SH, halo or carboxyl; said alkyl or alkenyl optionally containing at least one heteroatom selected independently from the group consisting of: O, S, and N; or
R₁₄ and R₁₄′ are independently C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: O, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: O, S, and N;
or R₁₄ and R₁₄′ are independently C₁₋₄ alkyl which when joined together with N form a 3 to 6-membered nitrogen-containing ring which is optionally fused with a further C₃₋₇ cycloalkyl, C₆ or C₁₀ aryl or heterocycle;
with the proviso that when Z is CH, then R₁₃ is not an α-amino acid or an ester thereof;
when Z is N, then R₁₃ is H; carboxy; C₁₋₆ alkyl optionally substituted with carboxy; CH₂—R₁₄; CHR₁₄R₁₄′; CH(F)—R₁₄; O—R₁₄; NR₁₄R₁₄′ or S—R₁₄ wherein R₁₄ and R₁₄′ are as defined above; or
Q is a phosphonate group of the formula: 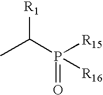
wherein R₁₅ and R₁₆ are independently C₆₋₂₀ aryloxy; and R₁ is as defined above.

3. A method for treating diseases or disorders associated with cathepsin activity and/or for inhibiting cathepsin activity in a subject, wherein the method comprises administering to a subject in need of such treatment at least one HCV protease inhibitor in a pharmaceutically effective amount thereof, wherein the dosage formulation is capable of maintaining an average Cmin plasma concentration of the at least one HCV protease inhibitor at or above 10 ng/ml,
wherein the at least one HCV protease inhibitor is selected from the group consisting of compounds of Formulae I to XXVI below:
a. Formula I 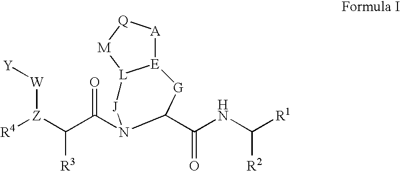
or a pharmaceutically acceptable salt, solvate or ester thereof,
wherein in Formula I above:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is H, OH, OR⁸, NR⁹R¹⁰, CF₃, C₂F₅, C₃F₇, CF₂R⁶, R⁶, or COR⁷ wherein R⁷ is H, OH, OR⁸, CHR⁹R¹⁰, or NR⁹R¹⁰, wherein R⁶, R⁸, and R¹⁰ are independently selected from the group consisting of H, alkyl, aryl, heteroalkyl, heteroaryl, cycloalkyl, cycloalkyl, arylalkyl, heteroarylalkyl, [CH(R^{1′})]ₚCOOR¹¹, [CH(R^{1′})]ₚCONR¹²R¹³, [CH(R^{1′})]ₚSO₂R¹¹, [CH(R^{1′})]ₚCOR¹¹, [CH(R^{1′})]ₚCH(OH)R¹¹, CH(R^{1′})CONHCH(R²)COOR¹¹, CH(R^{1′})CONHCH(R^{2′})CONR¹²R¹³, CH(R^{1′})CONHCH(R²)R′, CH(R^{1′})CONHCH(R^{2′})CONHCH(R^{3′})COOR¹¹, CH(R^{1′})CONHCH(R^{2′})CONHCH(R^{3′})CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹ and CH(R^{1′})CONHCH(R^{2′})CONHCH(R^{3′})CONHCH(R^{4′})CONHCH(R^{5′}) CONR¹²R¹³, wherein R^{1′}, R^{2′}, R^{3′}, R^{4′}, R^{5′}, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;
Z is selected from O, N, CH or CR;
W maybe present or absent, and if W is present, W is selected from C═O, C═S, C(═N—CN), or SO₂;
Q maybe present or absent, and when Q is present, Q is CH, N, P, (CH₂)ₚ, (CHR)ₚ, (CRR′)ₚ, O, NR, S, or SO₂; and when Q is absent, M may be present or absent; when Q and M are absent, A is directly linked to L;
A is O, CH₂, (CHR)ₚ, (CHR—CHR′)ₚ, (CRR′)ₚ, NR, S, SO₂ or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;
L may be present or absent, and when L is present, L is CH, CR, O, S or NR; and when L is absent, then M may be present or absent; and if M is present with L being absent, then M is directly and independently linked to E, and J is directly and independently linked to E;
M may be present or absent, and when M is present, M is O, NR, S, SO₂, (CH₂)ₚ, (CHR)ₚ(CHR—CHR′)ₚ, or (CRR′)ₚ;
p is a number from 0 to 6; and
R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen;
(cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring
b. Formula II 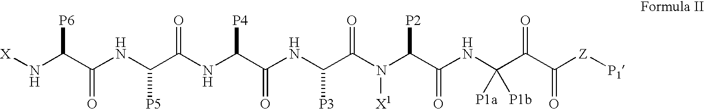
or a pharmaceutically acceptable salt, solvate or ester thereof;
wherein in Formula II above:
Z is O, NH or NR¹²;
X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylalkylcarbonyl, arylcarbonyl, heteroarylcarbonyl, alkoxycarbonyl, heterocyclyloxycarbonyl, aryloxycarbonyl, heteroaryloxycarbonyl, alkyaminocarbonyl, heterocyclylaminocarbonyl, arylaminocarbonyl, or heteroarylaminocarbonyl moiety, with the proviso that X may be additionally optionally substituted with R¹² or R¹³;
X¹ is H; C₁-C₄ straight chain alkyl; C₁-C₄ branched alkyl or; CH₂-aryl (substituted or unsubstituted);
R¹² is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R¹² may be additionally optionally substituted with R¹³.
R¹³ is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³.
P1a, P1b, P2, P3, P4, P5, and P6 are independently:
H; C1-C10 straight or branched chain alkyl; C2-C10 straight or branched chain alkenyl;
C3-C8 cycloalkyl, C3-C8 heterocyclic; (cycloalkyl)alkyl or (heterocyclyl)alkyl, wherein said cycloalkyl is made up of 3 to 8 carbon atoms, and zero to 6 oxygen, nitrogen, sulfur, or phosphorus atoms, and said alkyl is of 1 to 6 carbon atoms;
aryl, heteroaryl, arylalkyl, or heteroarylalkyl, wherein said alkyl is of 1 to 6 carbon atoms;
wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and
P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R¹³;
m. Formula III 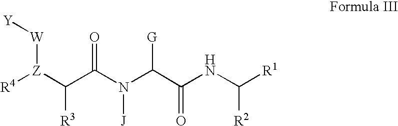
or a pharmaceutically acceptable salt, solvate or ester thereof;
wherein in Formula III above:
G, J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe additionally optionally substituted with X¹¹ or X¹²;
X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl, CH(R^{1′})COOR¹¹, CH(R^{1′})CONR¹²R¹³, CH(R^{1′})CONHCH(R^{2′})COOR¹¹, CH(R^{1′})CONHCH(R^{2′})CONR¹²R¹³, CH(R^{1′})CONHCH(R^{2′})R′, CH(R^{1′})CONHCH(R^{2′})CONHCH(R^{3′})COOR¹¹, CH(R^{1′})CONHCH(R^{2′})CONHCH(R^{3′})CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹, and CH(R^{1′})CONHCH(R^{2′})CONHCH(R^{3′})CONHCH(R^{4′})CONHCH(R^{5′})CONR¹²R¹³, wherein R^{1′}, R^{2′}, R^{3′}, R^{4′}, R^{5′}, R¹¹, R¹², R¹³, and R′ may be the same or different and are independently selected from a group consisting of H, alkyl, aryl, heteroalkyl, heteroaryl, cycloalkyl, alkyl-aryl, alkyl-heteroaryl, aryl-alkyl and heteroaralkyl;
Z is selected from O, N, or CH;
W maybe present or absent, and if W is present, W is selected from C═O, C═S, or SO₂; and
R, R′, R², R³ and R⁴ are independently selected from the group consisting of H; C1-C10 alkyl; C2-C10 alkenyl; C3-C8 cycloalkyl; C3-C8 heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate;
n. Formula IV 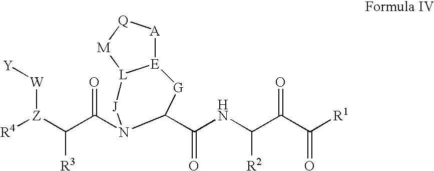
or a pharmaceutically acceptable salt, solvate or ester thereof;
wherein in Formula IV above:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is selected from the following structures: 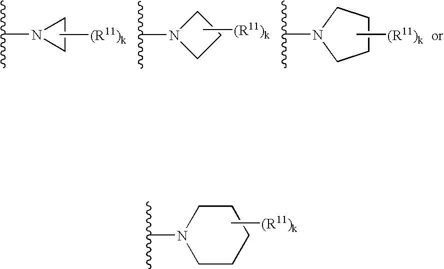
wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino,arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹≠H) maybe optionally substituted with X¹¹ or X¹²;
Z is selected from O, N, CH or CR;
W may be present or absent, and if W is present, W is selected from C═O, C═S, C(═N—CN), or S(O₂);
Q may be present or absent, and when Q is present, Q is CH, N, P, (CH₂)ₚ, (CHR)ₚ, (CRR′)ₚ, O, N(R), S, or S(O₂); and when Q is absent, M may be present or absent; when Q and M are absent, A is directly linked to L;
A is O, CH₂, (CHR)ₚ,(CHR—CHR′)ₚ, (CRR′)ₚ, N(R), S, S(O₂) or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J may be present or absent, and when J is present, J is (CH₂)ₚ, (CHR)ₚ, or (CRR′)ₚ, S(O₂), NH, N(R) or O; and when J is absent, G is present and E is directly linked to N shown in Formula I as linked to J;
L may be present or absent, and when L is present, L is CH, C(R), O, S or N(R); and when L is absent, then M may be present or absent; and if M is present with L being absent, then M is directly and independently linked to E, and J is directly and independently linked to E;
M may be present or absent, and when M is present, M is O, N(R), S, S(O₂), (CH₂)ₚ, (CHR)ₚ(CHR—CHR′)ₚ, or (CRR′)ₚ;
p is a number from 0 to 6; and
R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered cyclic ring structure or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring;
o. Formula V 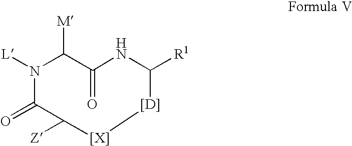
or a pharmaceutically acceptable salt, solvate or ester thereof,
wherein in Formula V above:
(1) R¹ is —C(O)R⁵ or —B(OR)₂;
(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;
(3) R⁷ is H, —OH, —OR⁸,or —CHR⁹R¹⁰;
(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R^{1′})CH(R^{1′})C(O)OR¹¹, [CH(R^{1′})]ₚC(O)OR¹¹, —[CH(R^{1′})]ₚC(O)NR¹²R¹³, —[CH(R^{1′})]ₚS(O₂)R¹¹, —[CH(R^{1′})]ₚC(O)R¹¹, —[CH(R^{1′})]ₚS(O₂)NR¹²R¹³, CH(R^{1′})C(O)N(H)CH(R^{2′})(R′), CH(R^{1′})CH(R^{1′})C(O)NR¹²R¹³, —CH(R^{1′})CH(R^{1′})S(O₂)R¹¹, —CH(R^{1′})CH(R^{1′})S(O₂)NR¹²R¹³, —CH(R^{1′})CH(R^{1′})C(O)R¹¹, —[CH(R^{1′})]ₚCH(OH)R¹¹, —CH(R^{1′})C(O)N(H)CH(R^{2′})C(O)OR¹¹, C(O)N(H)CH(R^{2′})C(O)OR¹¹, —C(O)N(H)CH(R^{2′})C(O)R¹¹, CH(R^{1′})C(O)N(H)CH(R^{2′})C(O)NR¹²R¹³, —CH(R^{1′})C(O)N(H)CH(R^{2′})R′, CH(R^{1′})C(O)N(H)CH(R^{2′})C(O)N(H)CH(R^{3′})C(O)OR¹¹, CH(R^{1′})C(O)N(H)CH(R^{2′})C(O)CH(R^{3′})NR¹²R¹³, CH(R^{1′})C(O)N(H)CH(R^{2′})C(O)N(H)CH(R^{3′})C(O)NR¹²R¹³, CH(R^{1′})C(O)N(H)CH(R^{2′})C(O)N(H)CH(R^{3′})C(O)N(H)CH(R^{4′})C(O)OR¹¹, CH(R^{1′l )C(O)N(H)CH(R}^{2′})C(O)N(H)CH(R^{3′})C(O)N(H)CH(R^{4′})C(O)NR¹²R¹³, CH(R^{1′})C(O)N(H)CH(R²)C(O)N(H)CH(R^{3′})C(O)N(H)CH(R^{4′})C(O)N(H)CH(R^{5′})C(O)OR¹¹, and CH(R^{1′})C(O)N(H)CH(R^{2′})C(O)N(H)CH(R^{3′})C(O)N(H)CH(R^{4′})C(O)N(H)CH(R^{5′})C(O)NR¹²R^{R}³;
wherein R^{1′}, R^{2′}, R^{3′}, R^{4′}, R^{5′}, R¹¹, R¹² and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl; or
R¹² and R¹³ are linked together wherein the combination is cycloalkyl, heterocycloalkyl, ary or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain;
or L′ and M′ are linked together to form a ring structure wherein the portion of structural Formula 1 represented by 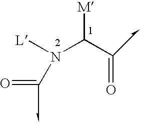
is represented by structural Formula 2: 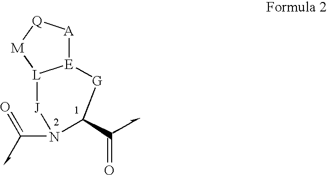
wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;
L is present or absent, and when L is present, L is C(H) or C(R); when L is absent, M is present or absent; if M is present with L being absent, then M is directly and independently linked to E, and J is directly and independently linked to E;
G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;
Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent being selected from —OR, —CH(R)(R′), S(O)₀₋₂R or —NRR′ or (iii) absent; when both Q and M are absent, A is either directly linked to L, or A is an independent substituent on E, said independent substituent bing selected from —OR, —CH(R)(R′), S(O)₀₋₂R or —NRR′ or A is absent;
A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, O, OR, N(R), S, S(O₂), (CH₂)ₚ, (CHR)ₚ (CHR—CHR′)ₚ, or (CRR′)ₚ; or M is linked to A to form an alicyclic, aliphatic or heteroalicyclic bridge;
(8) Z′ is represented by the structural Formula 3: 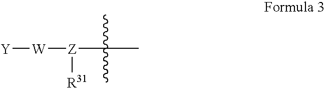
wherein in Formula 3, Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹¹ is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
Z is O, N, C(H) or C(R);
R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
W may be present or absent, and if W is present, W is C(═O), C(═S), C(═N—CN), or S(O₂);
(9) X is represented by structural Formula 4: 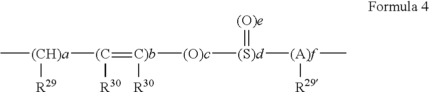
wherein in Formula 4, a is 2, 3, 4, 5, 6, 7, 8 or 9;
b, c, d, e and f are 0, 1, 2, 3, 4 or 5;
A is C, N, S or O;
R²⁹ and R^{29′} are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R²⁹ and R^{29′}are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;
(10) D is represented by structural Formula 5: 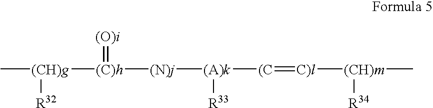
wherein in Formula 5, R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R³² and R³⁴ are linked together such that the combination forms a portion of a cycloalkyl group;
g is 1, 2, 3, 4, 5, 6, 7, 8 or 9;
h, i, j, k, l and m are 0, 1, 2, 3, 4 or 5; and
A is C, N, S or O,
(11) provided that when structural Formula 2: 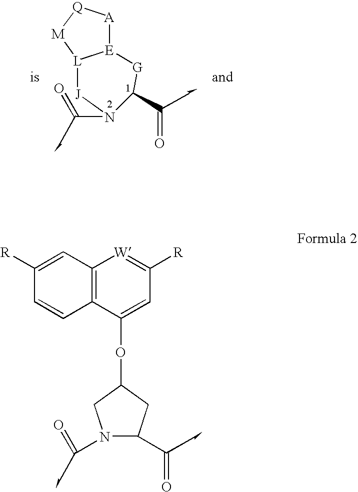
W′ is CH or N, both the following conditional exclusions (i) and (ii) apply:
conditional exclusion (i): Z′ is not —NH—R³⁶, wherein R³⁶ is H, C_{6 or 10} aryl, heteroaryl,
—C(O)—R³⁷, —C(O)—OR³⁷ or —C(O)—NHR³⁷, wherein R³⁷ is C₁₋₆ alkyl or C₃₋₆ cycloalkyl; and
conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈ alkyl, C₃₋₆ cycloalkyl, C_{6 to 10} aryl or C₇₋₁₆ aralkyl;
p. Formula VI 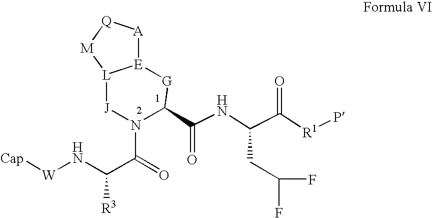
or a pharmaceutically acceptable salt, solvate or ester of said compound, wherein in Formula VI above:
Cap and P′ are independently H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino can be unsubstituted or optionally independently substituted with one or two substituents which can be the same or different and are independently selected from X¹ and X²;
X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;
X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;
W may be present or absent, and when W is present W is C(═O), C(═S), C(═NH), C(═N—OH), C(═N—CN), S(O) or S(O₂);
Q maybe present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R′), S(O)₀₋₂R or —NRR′; when both Q and M are absent, A is either directly linked to L, or A is an independent substituent on E, selected from —OR, CH(R)(R′), —S(O)₀₋₂R or —NRR′;
A is present or absent and if present A is —O—, —O(R) CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either Q and E are connected by a bond or Q is an independent substituent on M;
E is present or absent and if present E is CH, N, C(R);
G may be present or absent, and when G is present, G is (CH₂)ₚ, (CHR)ₚ, or (CRR′)ₚ; when G is absent, J is present and E is directly connected to the carbon atom marked position 1;
J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
L may be present or absent, and when L is present, L is CH, N, or CR; when L is absent, M is present or absent; if M is present with L being absent, then M is directly and independently linked to E, and J is directly and independently linked to E;
M may be present or absent, and when M is present, M is O, N(R), S, S(O₂), (CH₂)ₚ, (CHR)ₚ, (CHR—CHR′)ₚ, or (CRR′)ₚ;
p is a number from 0 to 6;
R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;
R and R′ in (CRR′) can be linked together such that the combination forms a cycloalkyl or heterocyclyl moiety; and
R¹ is N(R) or O;
q. Formula VII 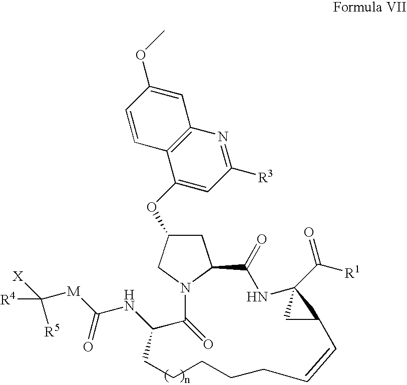
or a pharmaceutically acceptable salt, solvate or ester thereof, wherein in Formula VII above:
M is O, N(H), or CH₂;
n is 0-4;
R¹ is —OR⁶, —NR⁶R⁷ or 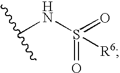
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 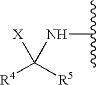
is represented by 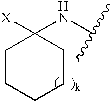
where k is 0 to 2;
X is selected from the group consisting of: 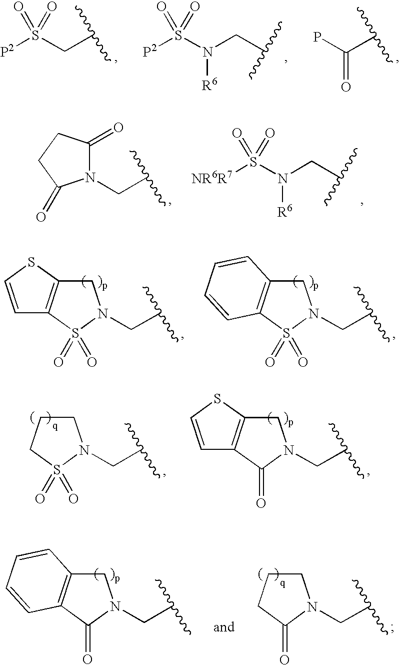
where p is 1 to 2, q is 1-3 and p² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 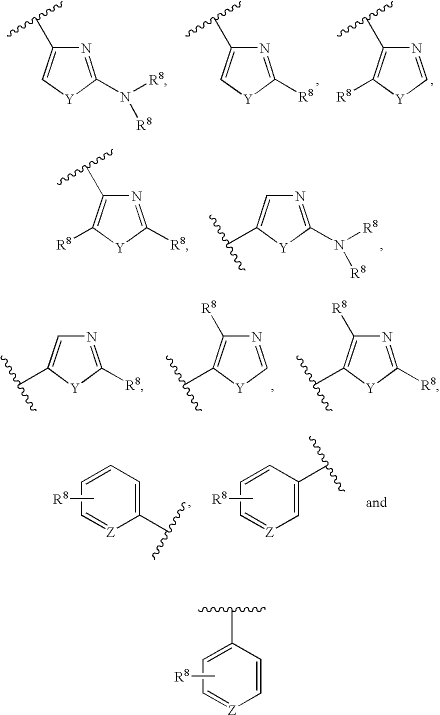
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
h. Formula VIII 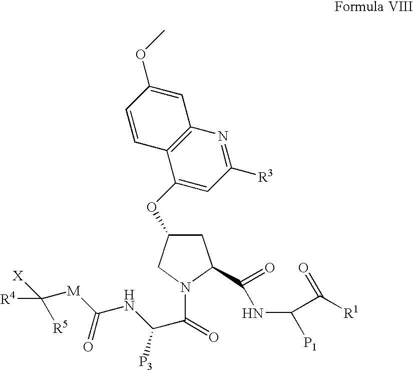
or a pharmaceutically acceptable salt, solvate or ester thereof, wherein in Formula VIII above,
M is O, N(H), or CH₂;
R¹ is —OR⁶, —NR⁶R⁷ or 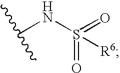
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
P₁ is selected from the group consisting of alkyl, alkenyl, alkynyl, cycloalkyl haloalkyl;
P₃ is selected from the group consisting of alkyl, cycloalkyl, aryl and cycloalkyl fused with aryl;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 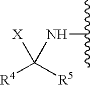
is represented by 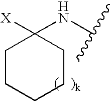
where k is 0 to 2;
X is selected from the group consisting of: 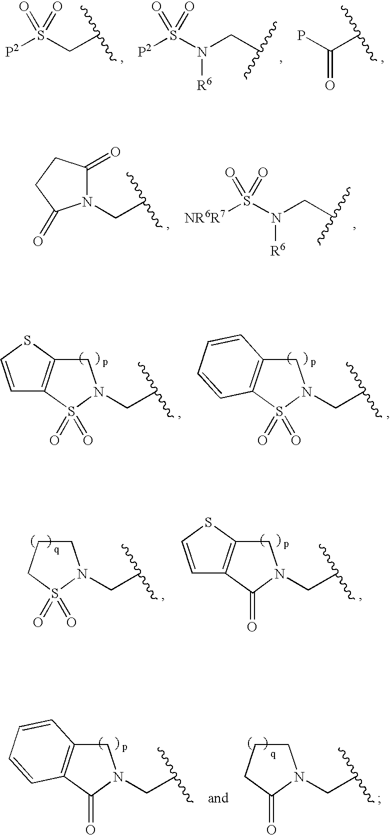
where p is 1 to 2, q is 1 to 3 and p² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 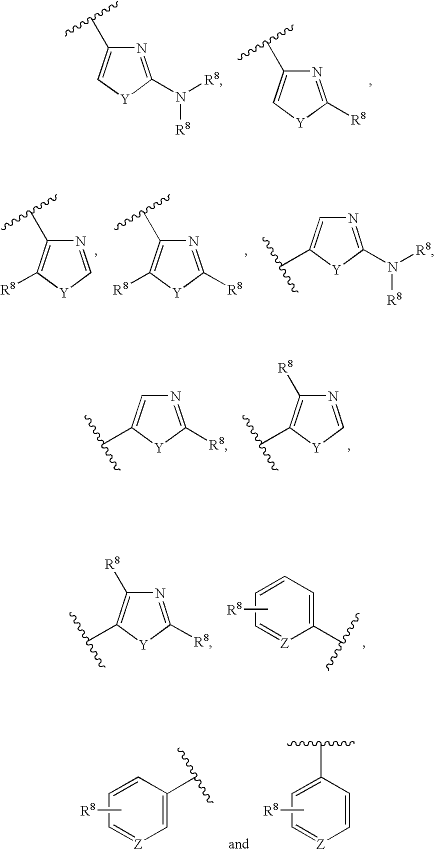
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
i. Formula IX 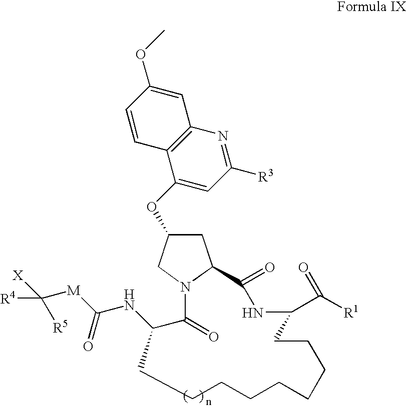
or a pharmaceutically acceptable salt, solvate or ester thereof, wherein in Formula IX above,
M is O, N(H), or CH₂;
n is 0-4;
R¹ is —OR⁶, —NR⁶R⁷ or 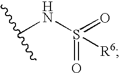
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 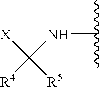
is represented by 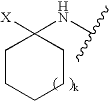
where k is 0 to 2;
X is selected from the group consisting of: 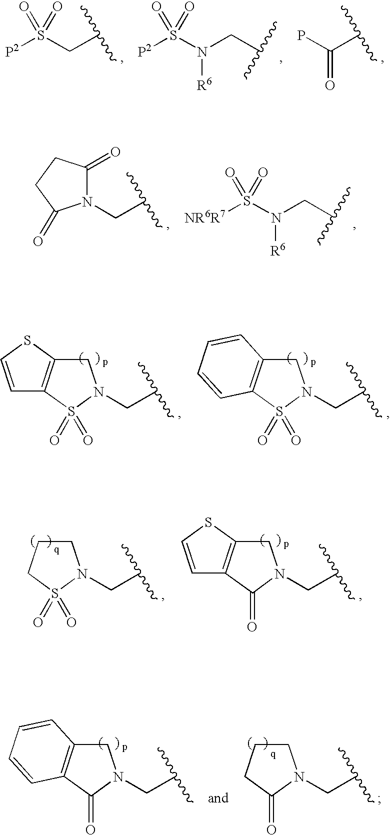
where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 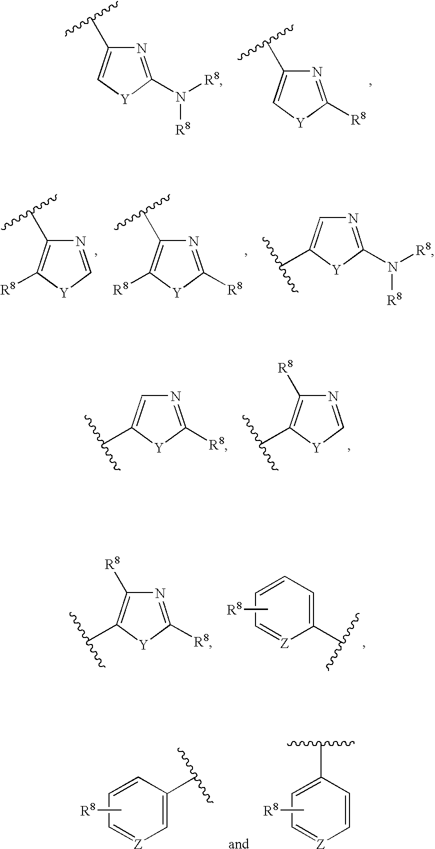
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy;
z. Formula X 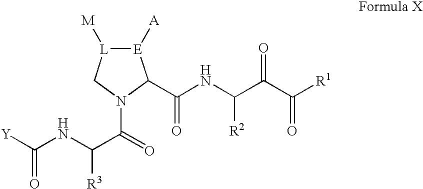
or a pharmaceutically acceptable salt, solvate or ester thereof;
wherein in Formula X above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 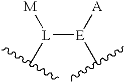
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R² and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 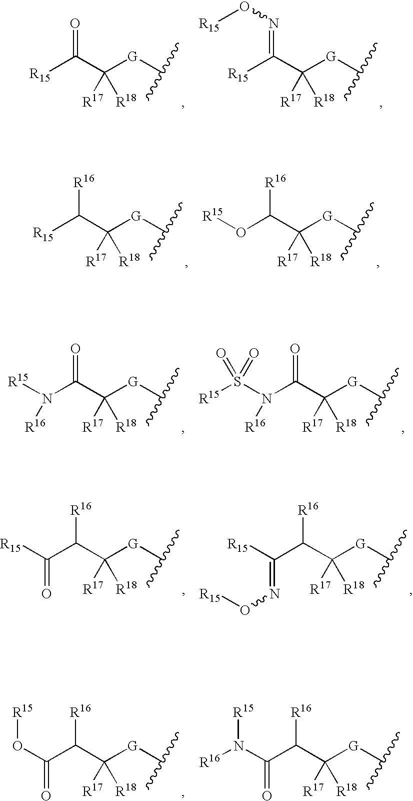
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
aa. Formula XI 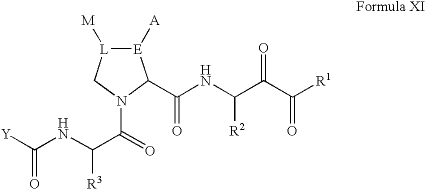
or a pharmaceutically acceptable salt, solvate or ester thereof; wherein in Formula XI above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, NR⁹R¹⁰, SR, SO₂R, and halo; or A and M are connected to each other (in other words, A-E-L-M taken together) such that the moiety: 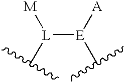
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R² and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;
Y is selected from the following moieties: 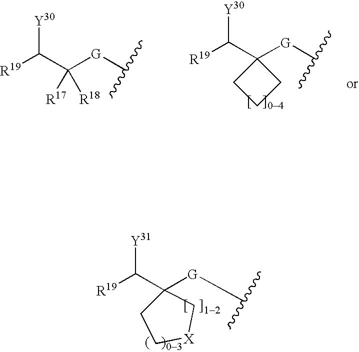
wherein Y³⁰ and Y³¹ are selected from 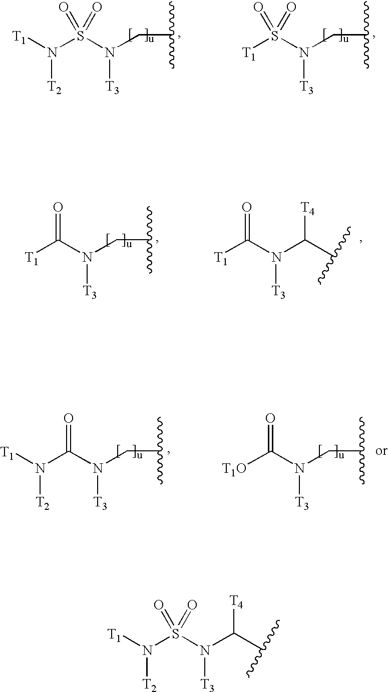
where u is a number 0-6;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, T₃ and T₄ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
bb. Formula XII 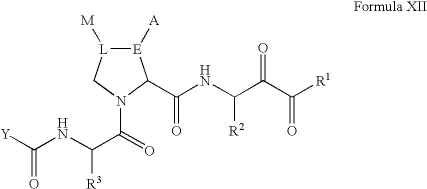
or a pharmaceutically acceptable salt, solvate or ester thereof; wherein in Formula XII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 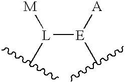
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 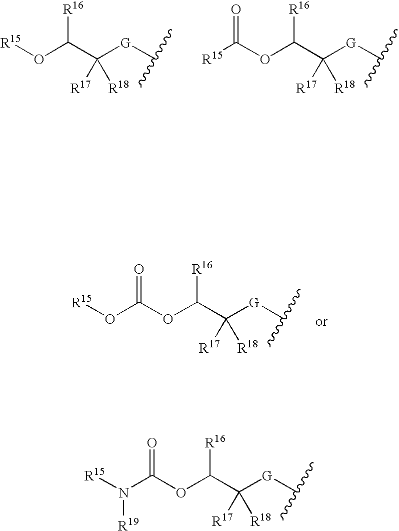
wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro;
cc. Formula XIII 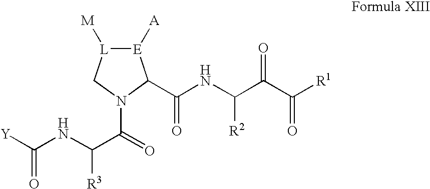
or a pharmaceutically acceptable salt, solvate or ester thereof; wherein in Formula XIII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other (in other words, A-E-L-M taken together) such that the moiety: 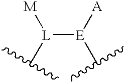
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 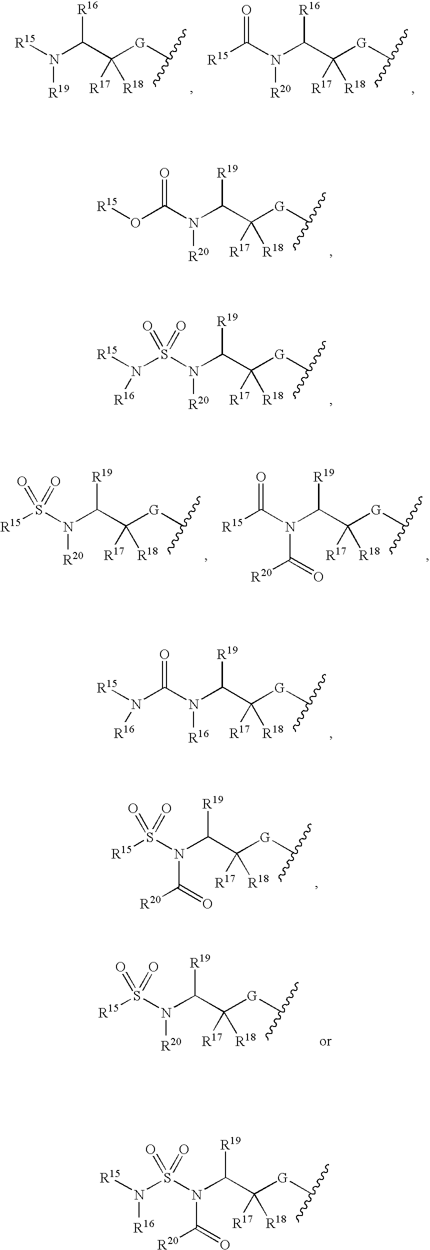
wherein G is NH or O, and R¹⁵, R¹⁶, R¹⁷,R¹⁸, R¹⁹ and R²⁰ can be the same or different, each being independently selected from the group consisting of H, C₁-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
dd. Formula XIV 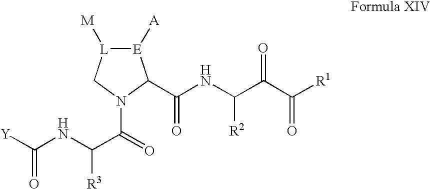
or a pharmaceutically acceptable salt, solvate or ester thereof; wherein in Formula XIV above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo;
or A and M are connected to each other such that the moiety: 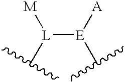
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected 5 from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;
and Y is selected from the following moieties: 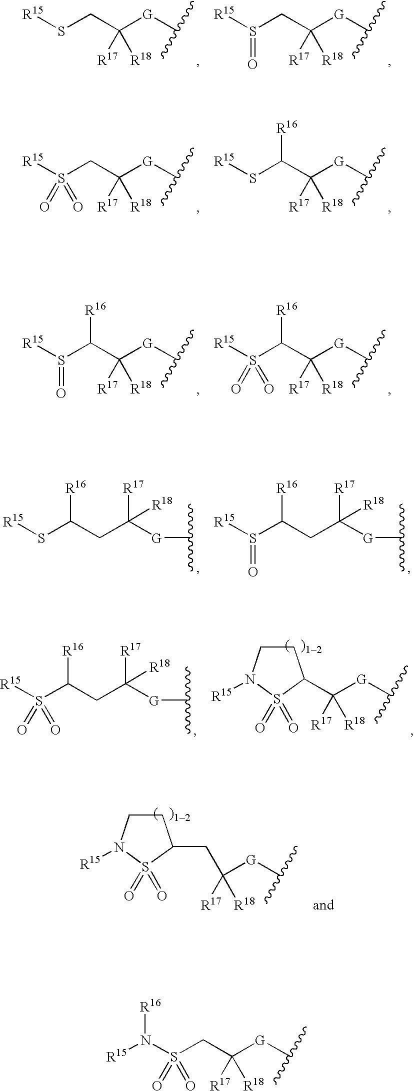
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro;
ee. Formula XV 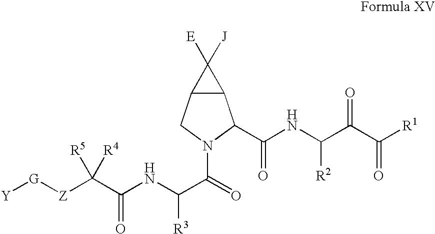
or a pharmaceutically acceptable salt, solvate or ester thereof; wherein in Formula XV above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, aryl-, heteroalkyl-, heteroaryl-, cycloalkyl-, cycloalkyl-, arylalkyl-, and heteroarylalkyl;
E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;
Z is N(H), N(R), or O, with the proviso that when Z is O, G is present or absent and if G is present with Z being O, then G is C(═O);
G maybe present or absent, and if G is present, G is C(═O) or S(O₂), and when G is absent, Z is directly connected to Y;
Y is selected from the group consisting of: 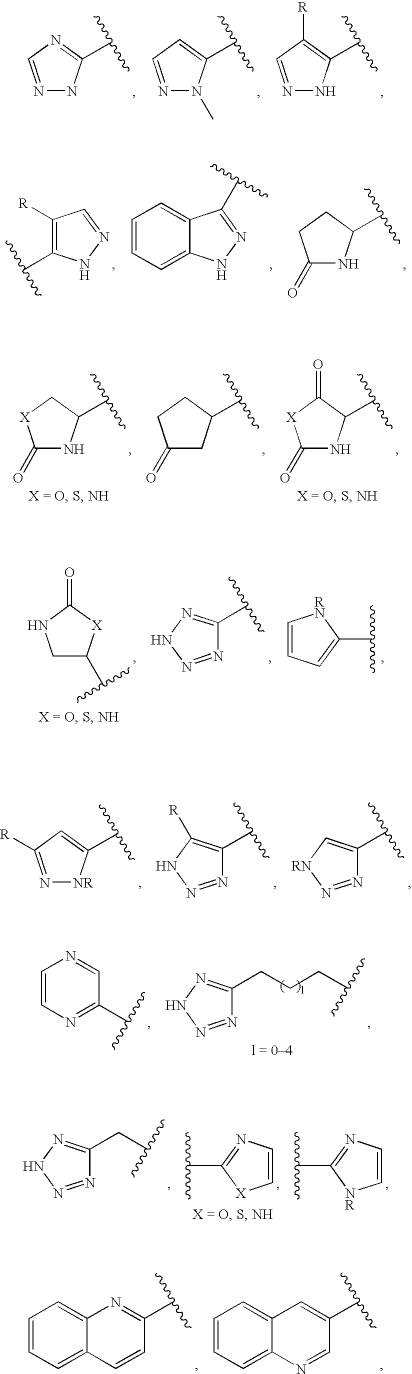 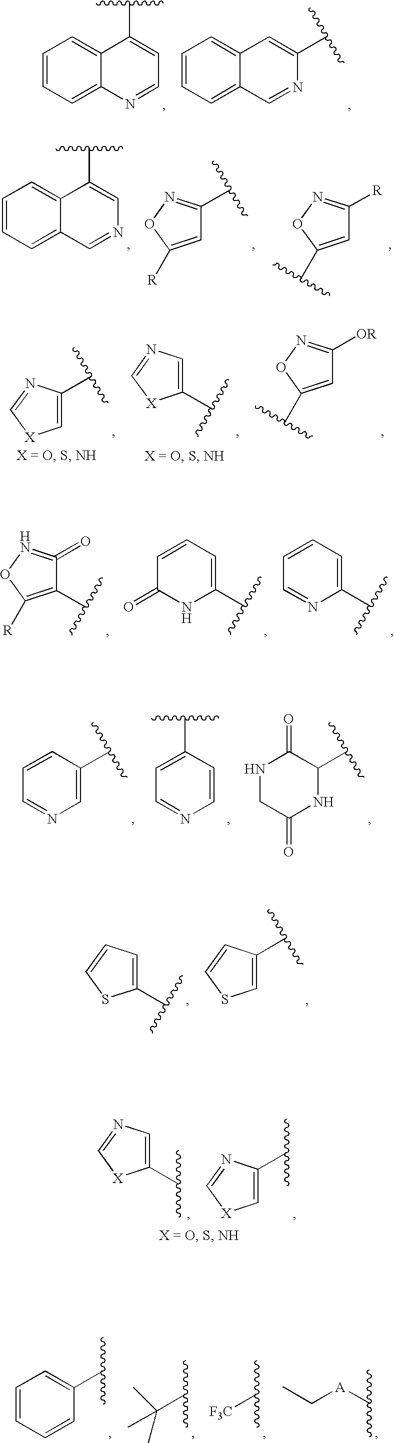 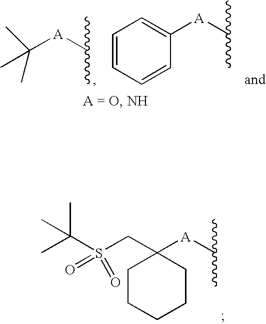
R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;
wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
ff. Formula XVI 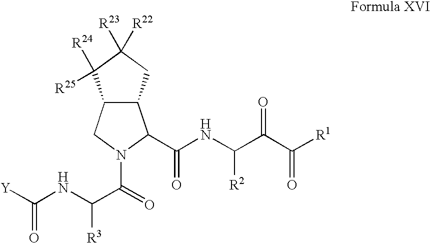
or a pharmaceutically acceptable salt, solvate or ester thereof; wherein in Formula XVI above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 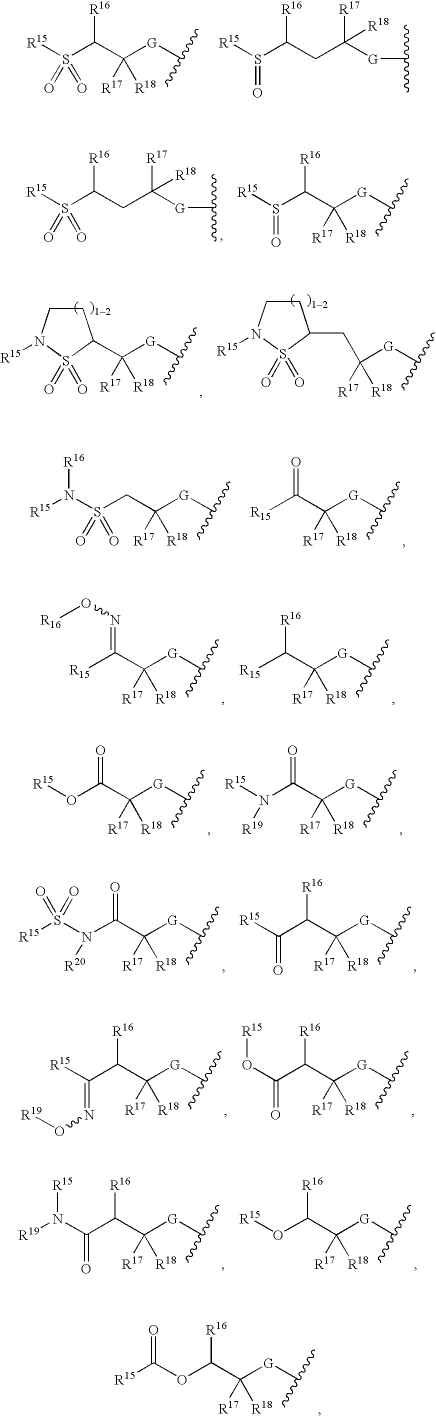 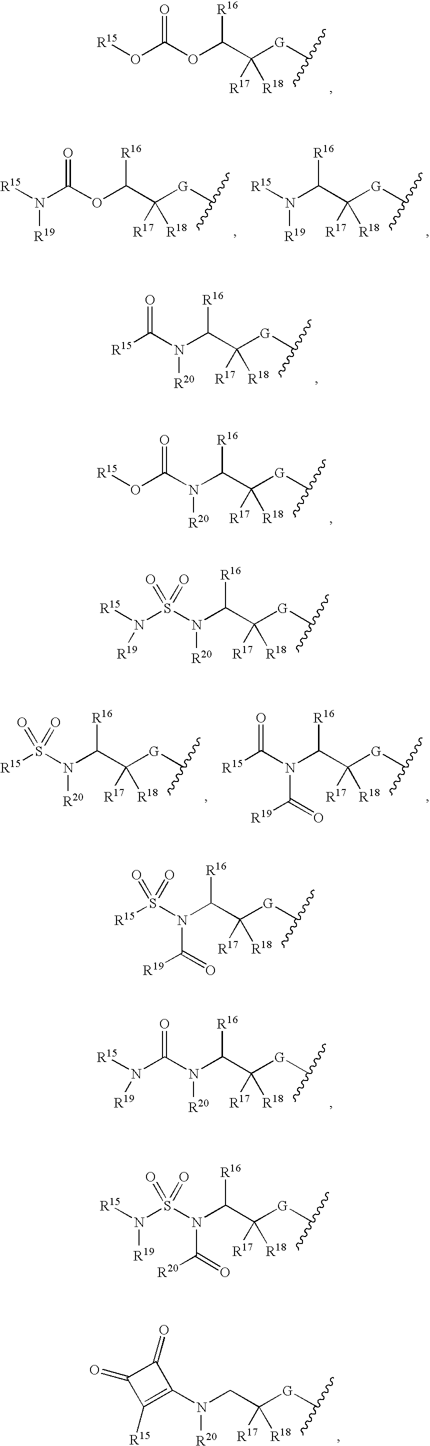 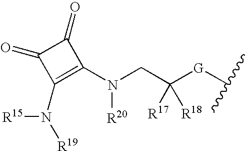
wherein G is NH or O; and R¹⁵, R¹⁶, R¹⁷, R¹⁸, R¹⁹, R²⁰, R²¹, R²², R²³, R²⁴ and R²⁵ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
gg. Formula XVII 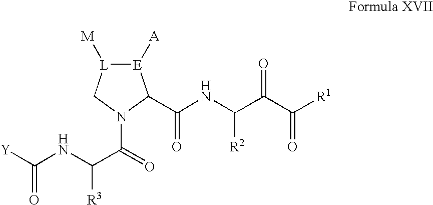
or a pharmaceutically acceptable salt, solvate or ester thereof; wherein in Formula XVII above:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other such that the moiety: 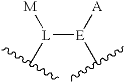
shown above in Formula I forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
Y is selected from the following moieties: 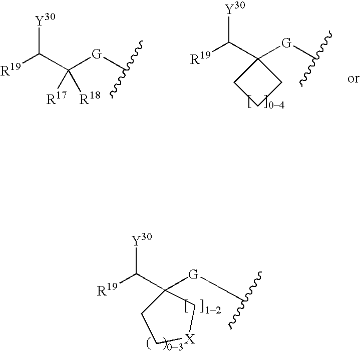
wherein Y³⁰ is selected from 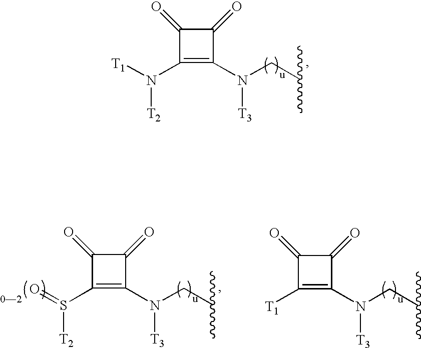
where u is a number 0-1;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
r. Formula XVIII 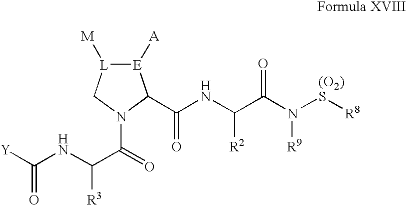
or a pharmaceutically acceptable salt, solvate or ester thereof, wherein in Formula XVIII above:
R⁸ is selected from the group consisting of alkyl-, aryl-, heteroalkyl-, heteroaryl-, cycloalkyl-, heterocyclyl-, arylalkyl-, heteroarylalkyl- , and heterocyclylalkyl;
R⁹ is selected from the group consisting of H, alkyl, alkenyl, alkynyl, aryl and cycloalkyl;
A and M can be the same or different, each being independently selected from R, OR, N(H)R, N(RR′), SR, S(O₂)R, and halo; or A and M are connected to each other (in other words, A-E-L-M taken together) such that the moiety: 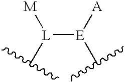
shown above in Formula I forms either a three, four, five, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl;
R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;
Y is selected from the following moieties: 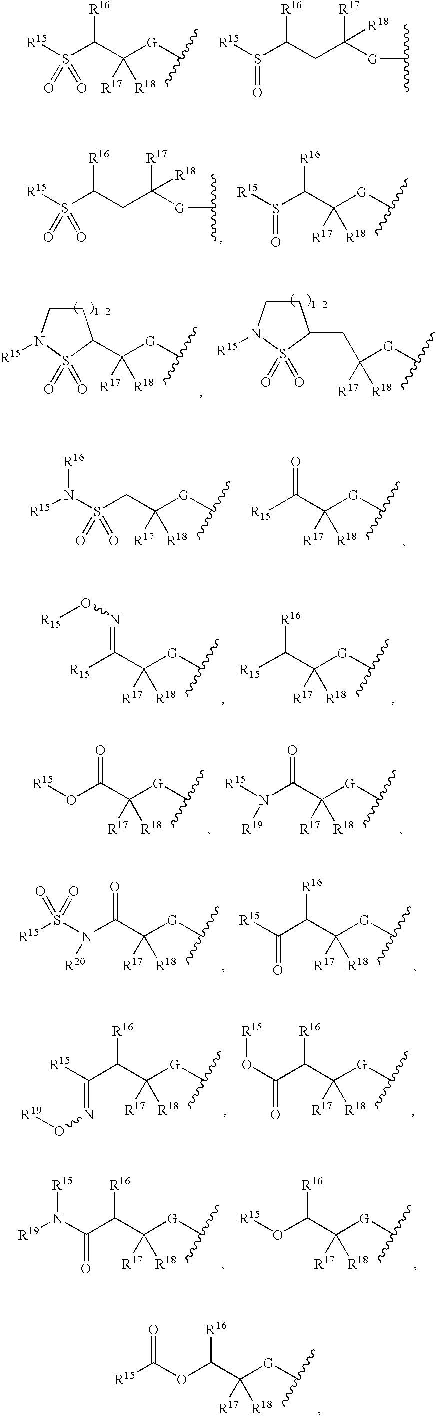 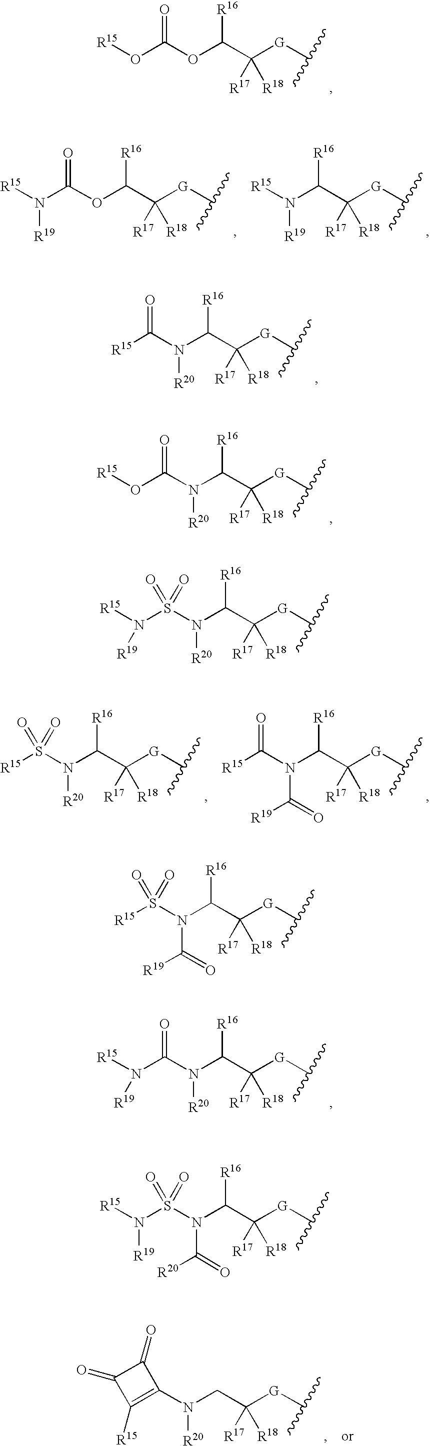 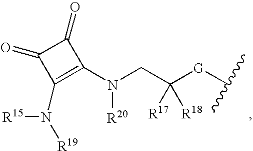
wherein G is NH or O; and R¹⁵, R¹⁶, R¹⁷, R¹⁸, R¹⁹ and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl, spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
s. Formula XIX 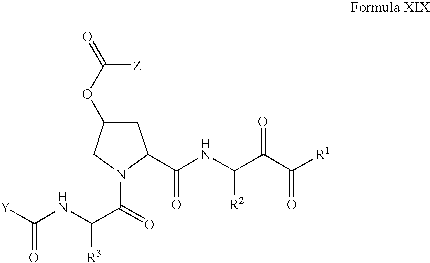
wherein in Formula XIX above:
Z is selected from the group consisting of a heterocyclyl moiety,
N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N(heteroaryl)₂;
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 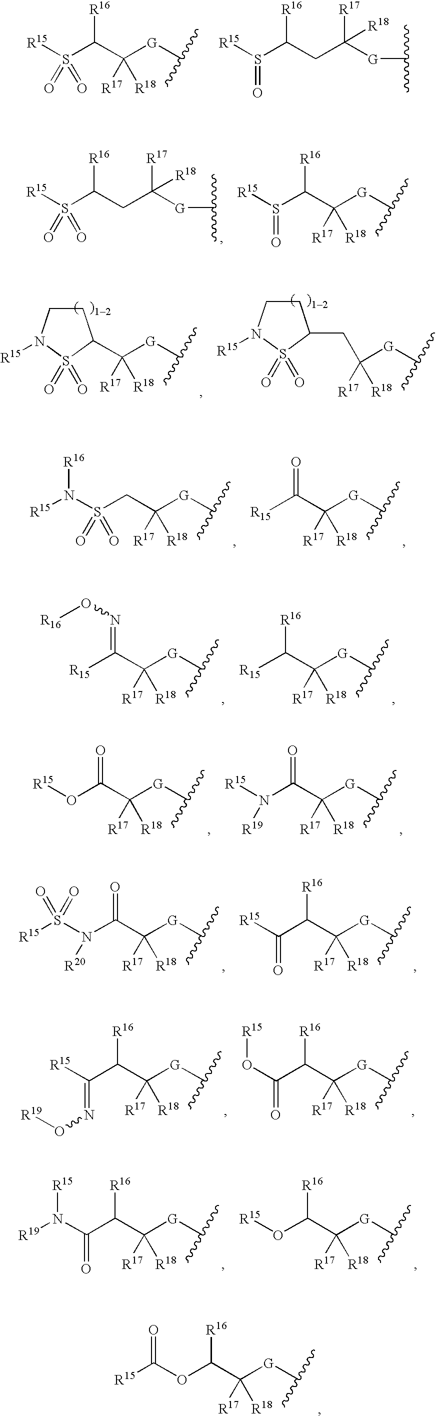 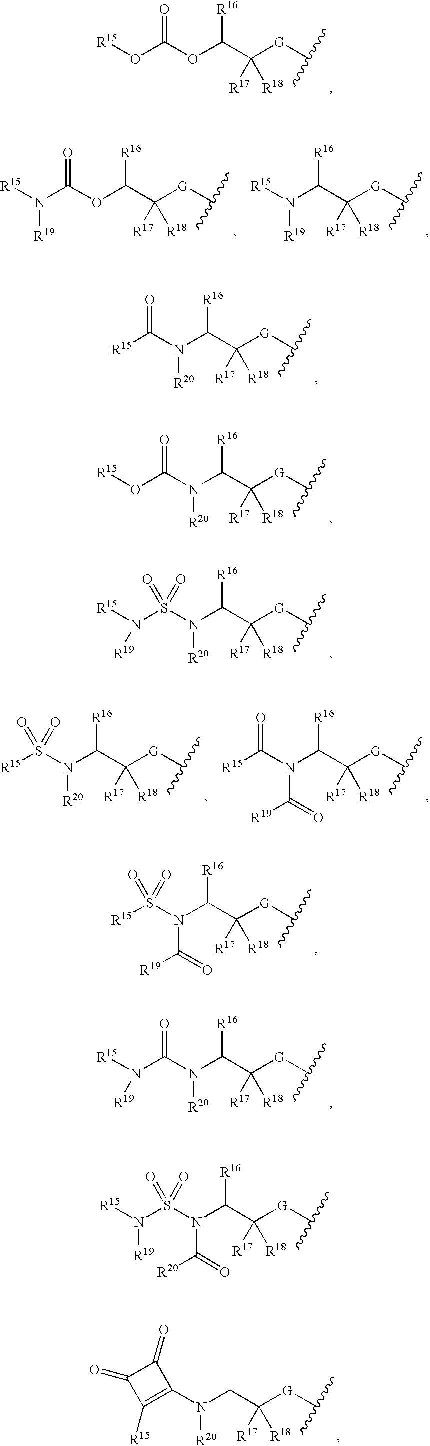 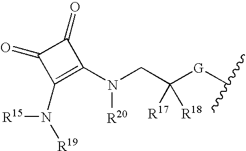
wherein G is NH or O; and R¹⁵, R¹⁶, R¹⁷, R¹⁸, R¹⁹, R²⁰ and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro;
v. Formula XX 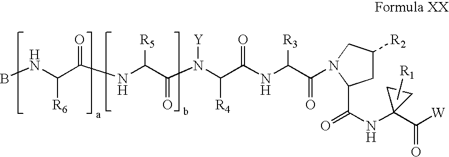
or a pharmaceutically acceptable salt, solvate or ester thereof; wherein in Formula XX above:
a is 0 or 1; b is 0 or 1; Y is H or C₁₋₆ alkyl;
B is H, an acyl derivative of formula R₇—C(O)— or a sulfonyl of formula R₇—SO2 wherein
R7 is (i) C_{I-10} alkyl optionally substituted with carboxyl, C₁₋₆ alkanoyloxy or C₁₋₆ alkoxy;
(ii) C₃₋₇ cycloalkyl optionally substituted with carboxyl, (C_{I-6} alkoxy)carbonyl or phenylmethoxycarbonyl;
(iii) C₆ or C₁₀ aryl or C₇₋₁₆ aralkyl optionally substituted with C₁₋₆ alkyl, hydroxy, or amino optionally substituted with C₁₋₆ alkyl; or
(iv) Het optionally substituted with C₁₋₆ alkyl, hydroxy, amino optionally substituted with C₁₋₆ alkyl, or amido optionally substituted with C₁₋₆ alkyl;
R₆, when present, is C₁₋₆ alkyl substituted with carboxyl;
R₅, when present, is C₁₋₆ alkyl optionally substituted with carboxyl;
R₄ is C₁₋₁₀ alkyl, C₃₋₇ cycloalkyl or C₄₋₁₀ (alkylcycloalkyl);
R₃ is C₁₋₁₀ alkyl, C₃₋₇ cycloalkyl or C₄₋₁₀ (alkylcycloalkyl);
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri-substituted with R₂₁,
or R₂₀ is Het or (lower alkyl)-Het optionally mono-, di- or tri-substituted with R₂₁, wherein each R₂₁ is independently C₁₋₆ alkyl; C₁₋₆ alkoxy; amino optionally mono- or di-substituted with C₁₋₆ alkyl; sulfonyl; NO₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;
wherein R₂₂ is C₁₋₆ alkyl; C₁₋₆ alkoxy; amino optionally mono- or di-substituted with C₁₋₆ alkyl; sulfonyl; NO₂; OH; SH; halo; haloalkyl; carboxyl; amide or (lower alkyl)amide;
R₁ is C₁₋₆ alkyl or C₂₋₆ alkenyl optionally substituted with halogen; and
W is hydroxy or a N-substituted amino;
u. Formula XXI: 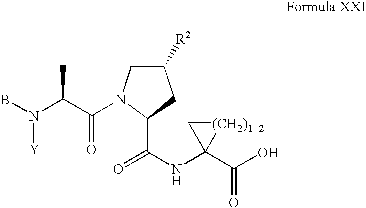
or a pharmaceutically acceptable salt, solvate or ester thereof; wherein in Formula XXI above:
B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl of formula R₄—O—C(O)—; an amide of formula R₄—N(R₅)—C(O)—; a thioamide of formula R₄—N(R₅)—C(S)—; or a sulfonyl of formula R₄—SO2 wherein
R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;
(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;
(iii) amino optionally mono- or di-substituted with C₁₋₆ alkyl; amido; or (lower alkyl)amide;
(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or
(v) Het or (lower alkyl)-Het, both optionally substituted with C₁₋₆ alkyl, hydroxy, amido, (lower alkyl) amide, or amino optionally mono- or di-substituted with C₁₋₆ alkyl;
R₅ is H or C₁₋₆ alkyl;
with the proviso that when R₄ is an amide or a thioamide, R₄ is not (ii) a cycloalkoxy;
Y is H or C₁₋₆ alkyl;
R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,
or R₂₀ is Het or (lower alkyl)-Het, both optionally mono-, di- or tri-substituted with R₂₁,
wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;
wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;
R1 is H; C₁₋₆ alkyl, C₃₋₇ cycloalkyl, C₂₋₆ alkenyl, or C₂₋₆ alkynyl, all optionally substituted with halogen;
v. Formula XXII: 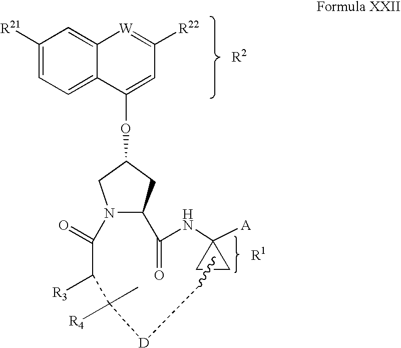
or a pharmaceutically acceptable salt, solvate or ester thereof; wherein in Formula XXII above:
W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂, wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵, wherein each R²⁵ is independently: H, C₁₋₆ alkyl or C₃₋₆ cycloalkyl; or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹, wherein
R⁴¹ is H, C₁₋₆ alkyl, C₃₋₆ cycloalkyl or —C(O)—R⁴²,
wherein R⁴² is C₁₋₆ alkyl, C₃₋₆ cycloalkyl or C_{6 or 10} aryl;
R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C 1-6 thioalkyl, and
A is an amide of formula —C(O)—NH—R⁵, wherein R⁵ is selected from the group consisting of: C₁₋₈ alkyl, C₃₋₆ cycloalkyl, C_{6 or 10} aryl and C₇₋₁₆ aralkyl;
or A is a carboxylic acid;
w. Formula XXIII: 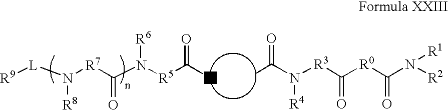
a pharmaceutically acceptable salt, solvate or ester thereof; wherein in Formula XXIII above:
R⁰ is a bond or difluoromethylene;
R¹ is hydrogen, optionally substituted aliphatic group, optionally substituted cyclic group or optionally substituted aromatic group;
R² and R⁹ are each independently optionally substituted aliphatic group, optionally substituted cyclic group or optionally substituted aromatic group;
R3, R5 and R7 are each independently:
optionally substituted (1,1- or 1,2-)cycloalkylene; or
optionally substituted (1,1- or 1,2-) heterocyclylene; or
methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or;
R4, R 6, R8 and R¹⁰ are each independently hydrogen or optionally substituted aliphatic group; 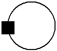
is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-(N(R⁸)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰OC(O)—, -S(O)₂—, or —NR¹⁰S(O)₂—; and n is 0 or 1, provided
when 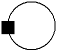
is substituted 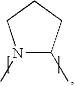
then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴ is optionally substituted aliphatic;
x. Formula XXIV: 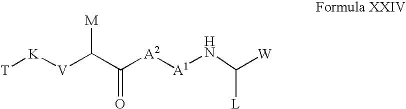
or a pharmaceutically acceptable salt, solvate or ester thereof; wherein in Formula XXIV above:
W is: 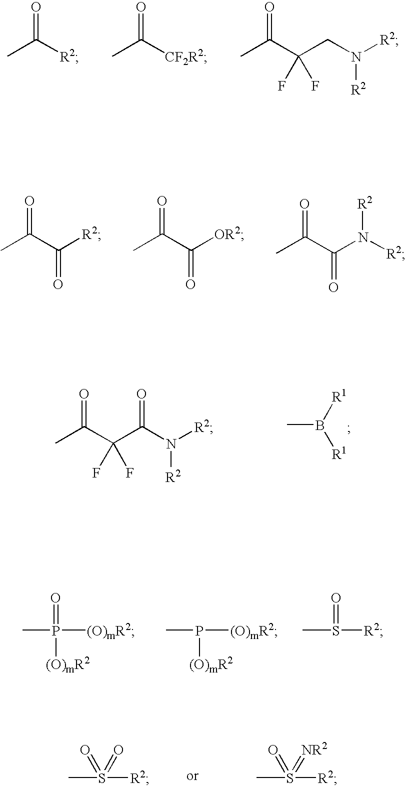
m is 0 or 1;
each R¹ is hydroxy, alkoxy, or aryloxy, or each R¹ is an oxygen atom and together with the boron, to which they are each bound, form a 5-7 membered ring, wherein the ring atoms are carbon, nitrogen, or oxygen;
each R² is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl, or two R² groups, which are bound to the same nitrogen atom, form together with that nitrogen atom, a 5-7 membered monocyclic heterocyclic ring system; wherein any R² carbon atom is optionally substituted with J;
J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;
J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoaikyl, halo, cyano, nitro, formyl, sulfonyl, or sulfonamido;
L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;
A¹ is a bond;
R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;
X is a bond, —C(H)(R7)-, —O—, —S—, or —N(R8)-;
R⁷ is hydrogen, alkyl, alkenyl, aryl, aralkyl, heterocyclyl, heterocyclylalkyl, heteroaryl, or heteroaralkyl, and is optionally substititued with 1-3 J groups;
R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R⁴, —SO₂R⁴, or carboxamido, and is optionally substititued with 1-3 J groups; or R⁸ and Z, together with the atoms to which they are bound, form a nitrogen containing mono- or bicyclic ring system optionally substituted with 1-3 J groups;
R¹⁴ is alkyl, aryl, aralkyl, heterocyclyl, heterocyclyalkyl, heteroaryl, or heteroaralkyl;
Y is a bond, —CH₂—, —C(O)—, —C(O)C(O)—, —S(O)—, —S(O)₂—, or —S(O)(NR⁷)—, wherein R⁷ is as defined above;
Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;
A² is a bond or 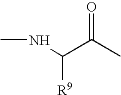
R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;
V is a bond, —CH₂—, —C(H)(R¹¹)—, —O—, —S—, or —N(R¹¹)—;
R¹¹ is hydrogen or C₁₋₃ alkyl;
K is a bond, —O—, —S—, —C(O)—, —S(O)—, —S(O)₂—, or —S(O)(NR¹¹)—, wherein R¹¹ is as defined above;
T is —R¹², -alkyl-R¹², -alkenyl-R¹², -alkynyl-R¹², —OR¹², —N(R¹²)2, —C(O)R¹², —C(═NOalkyl)R¹², or 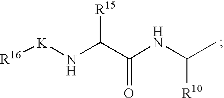
R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;
R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;
R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and
R¹⁶ is hydrogen, alkyl, aryl, heteroaryl, cycloalkyl, or heterocyclyl;
y. Formula XXV: 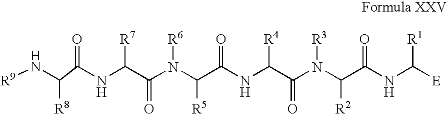
or a pharmaceutically acceptable salt, solvate or ester thereof;
wherein in Formula XVII above:
E represents CHO or B(OH)2;
R¹ represents lower alkyl, halo-lower alkyl, cyano-lower alkyl, lower alkylthio-lower alkyl, aryl-lower alkylthio-lower alkyl, aryl-lower alkyl, heteroaryllower alkyl, lower alkenyl or lower alkynyl;
R² represents lower alkyl, hydroxy-lower alkyl, carboxylower alkyl, aryl-lower alkyl, aminocarbonyl-lower alkyl or lower cycloalkyl-lower alkyl; and
R³ represents hydrogen or lower alkyl;
or R² and R³ together represent di- or trimethylene optionally substituted by hydroxy;
R4 represents lower alkyl, hydroxy-lower alkyl, lower cycloalkyl-lower alkyl, carboxy-lower alkyl, aryllower alkyl, lower alkylthio-lower alkyl, cyano-lower alkylthio-lower alkyl, aryl-lower alkylthio-lower alkyl, lower alkenyl, aryl or lower cycloalkyl;
R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;
R⁶ represents hydrogen or lower alkyl;
R⁷ represent lower alkyl, hydroxydower alkyl, carboxylower alkyl, aryl-iower alkyl, lower cycloalkyl-lower alkyl or lower cycloalkyl;
R⁸ represents lower alkyl, hydroxy-lower alkyl, carboxylower alkyl or aryl-lower alkyl; and
R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl; and
z. Formula XXVI: 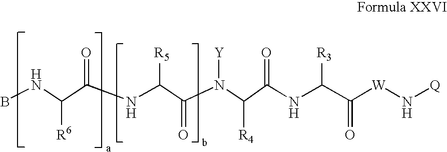
or a pharmaceutically acceptable salt, solvate or ester thereof; wherein in Formula XXVI above
B is an acyl derivative of formula R₁₁—C(O)— wherein R₁₁ is CI-10 alkyl optionally substituted with carboxyl; or R₁₁ is C₆ or C₁₀ aryl or C₇₋₁₆ aralkyl optionally substituted with a C₁₋₆ alkyl;
a is 0 or 1;
R₆, when present, is carboxy(lower)alkyl;
b is 0 or 1;
R₅, when present, is C₁₋₆alkyl, or carboxy(lower)alkyl;
Y is H or C₁₋₆ alkyl;
R₄ is C₁₋₁₀ alkyl; C₃₋₁₀ cycloalkyl;
R₃ is C1-10 alkyl; C₃₋₁₀ cycloalkyl;
W is a group of formula: 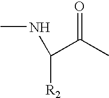
wherein R₂ is C₁₋₁₀ alkyl or C₃₋₇ cycloalkyl optionally substituted with carboxyl; C₆ or C₁₀ aryl; or C₇₋₁₆ aralkyl; or
W is a group of formula: 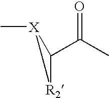
wherein X is CH or N; and
R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:
cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: O, S, and N; or
R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: O, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂. OH, SH, halo, carboxyl or carboxy(lower)alkyl; C₆ or C₁₀ aryl, or heterocycle; said second ring optionally containing at least one heteroatom selected independently from the group consisting of: O, S, and N;
Q is a group of the formula: 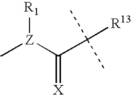
wherein Z is CH or N;
X is O or S;
R₁ is H, C₁₋₆ alkyl or C₁₋₆ alkenyl both optionally substituted with thio or halo; and
when Z is CH, then R₁₃ is H; CF₃; CF₂CF₃; CH₂—R₁₄; CH(F)—R₁₄; CF₂—R₁₄; NR₁₄R₁₄′; S—R₁₄; or CO—NH—R₁₄ wherein R₁₄ and R₁₄′ are independently hydrogen, cyclic C₃₋₁₀ alkyl or acyclic C₁₋₁₀ alkyl or cyclic C₃₋₁₀ alkenyl or acyclic C₂₋₁₀ alkenyl said alkyl or alkenyl optionally substituted with NH₂, OH, SH, halo or carboxyl; said alkyl or alkenyl optionally containing at least one heteroatom selected independently from the group consisting of: O, S, and N; or
R₁₄ and R₁₄′ are independently C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: O, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: O, S, and N;
or R₁₄ and R₁₄′ are independently C₁₋₄ alkyl which when joined together with N form a 3 to 6-membered nitrogen-containing ring which is optionally fused with a further C₃₋₇ cycloalkyl, C₆ or C₁₀ aryl or heterocycle;
with the proviso that when Z is CH, then R₁₃ is not an α-amino acid or an ester thereof;
when Z is N, then R₁₃ is H; carboxy; C₁₋₆ alkyl optionally substituted with carboxy; CH₂—R₁₄; CHR₁₄R₁₄′; CH(F)—R₁₄; O—R₁₄; NR₁₄R₄′ or S—R₁₄ wherein R₁₄ and R₁₄′ are as defined above; or
Q is a phosphonate group of the formula: 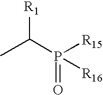
wherein R₁₅ and R₁₆ are independently C₆₋₂₀ aryloxy; and R₁ is as defined above.

4. The method of any of claims 1 or 2, wherein the at least one compound treats, prevents, and/or ameliorates disorders associated with HCV.

5. The method of any of claims 1 or 2, wherein the at least one compound treats and/or reduces signs and/or symptoms associated with HCV.

6. The method of any of claims 1 to 5, wherein the compound is selected from the group consisting of: 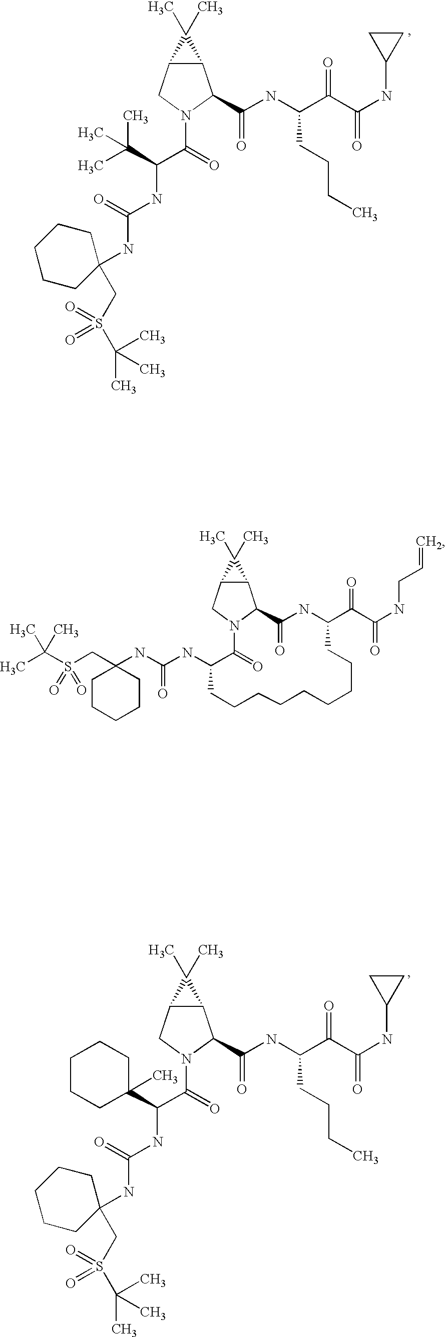 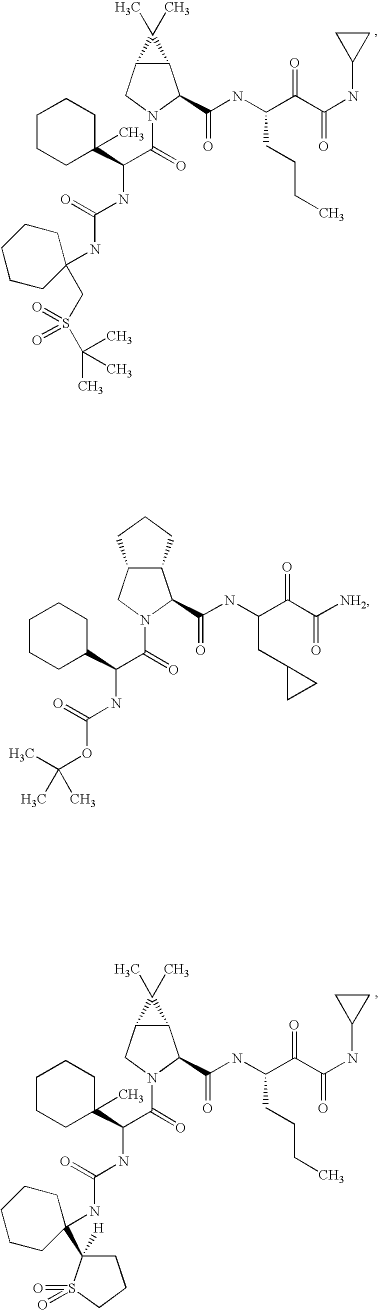 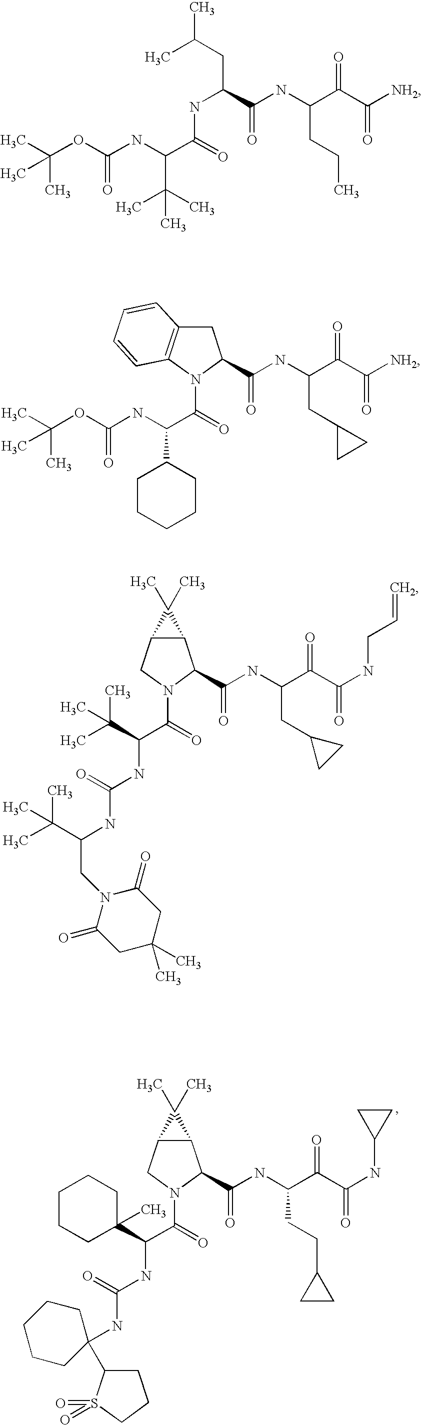 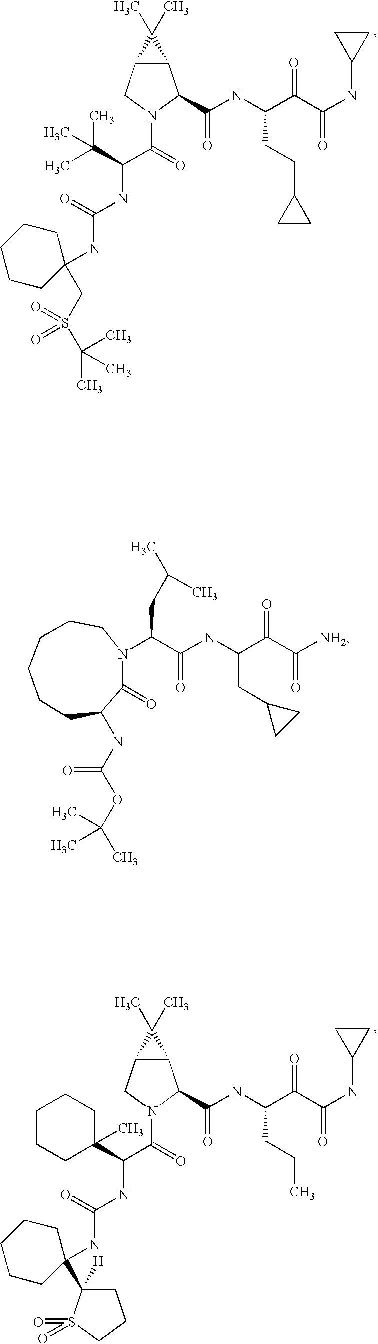 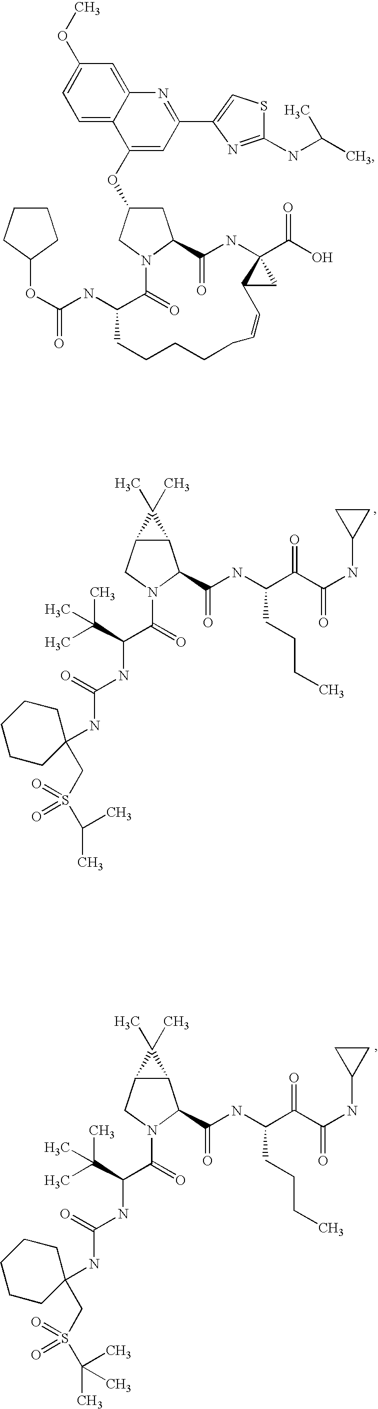 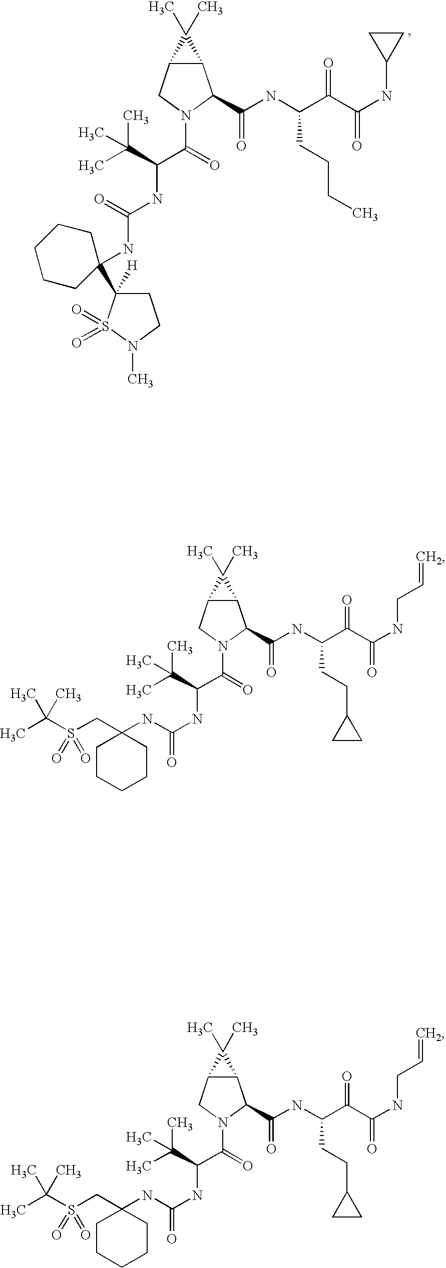 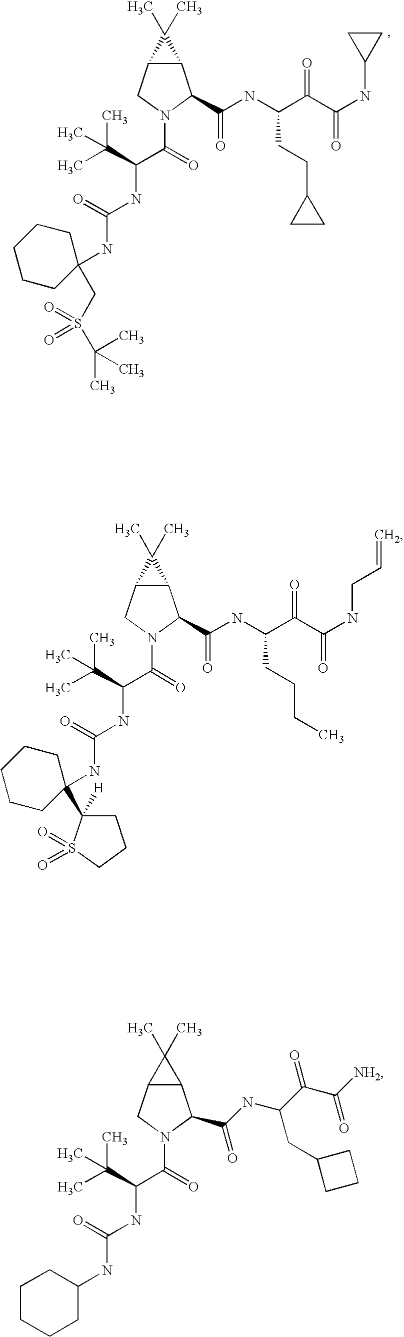 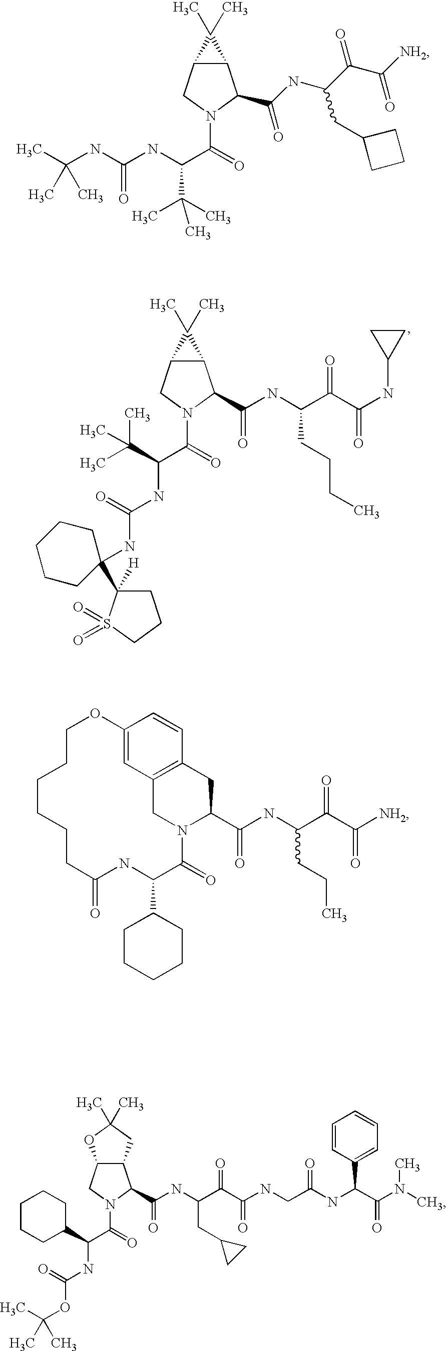 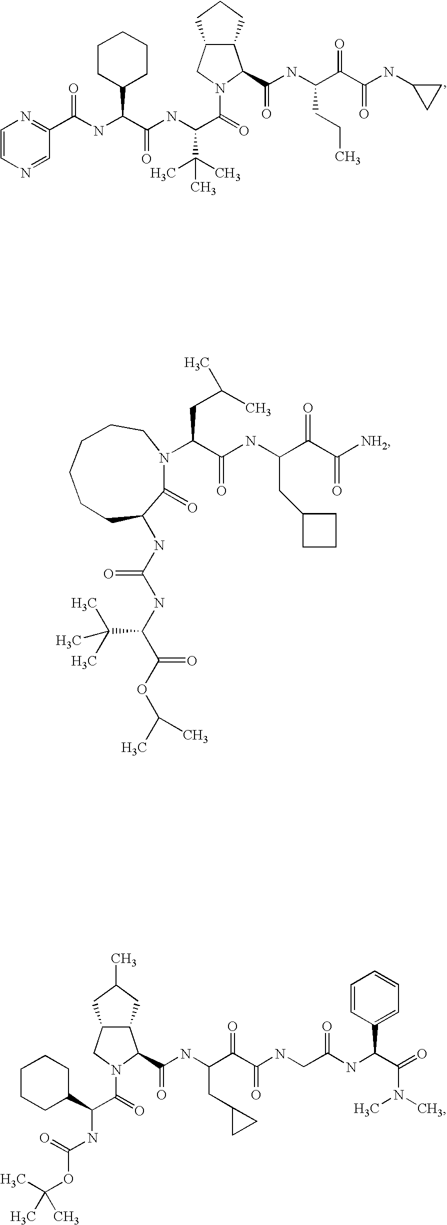 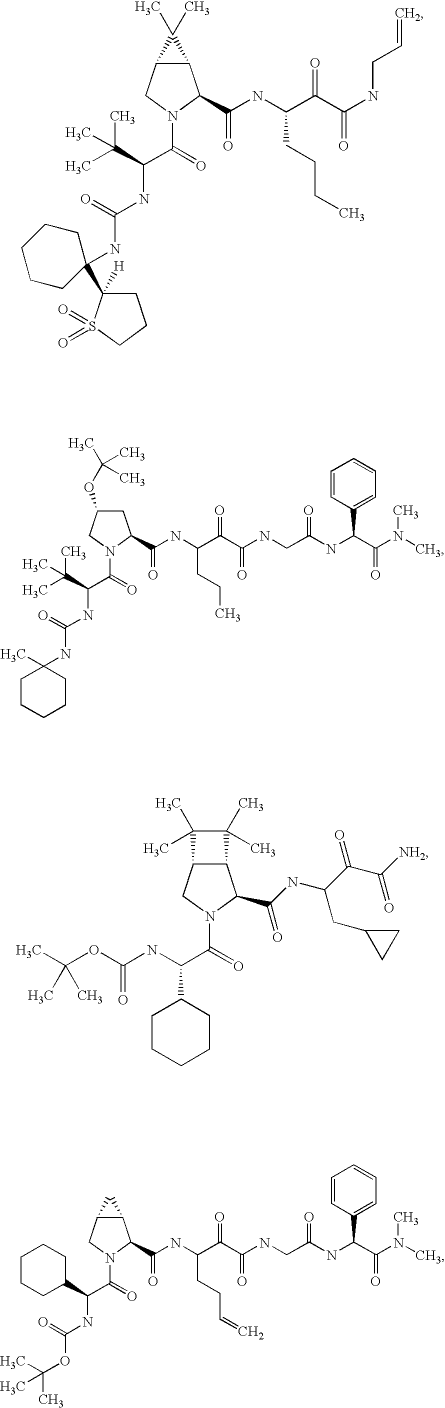 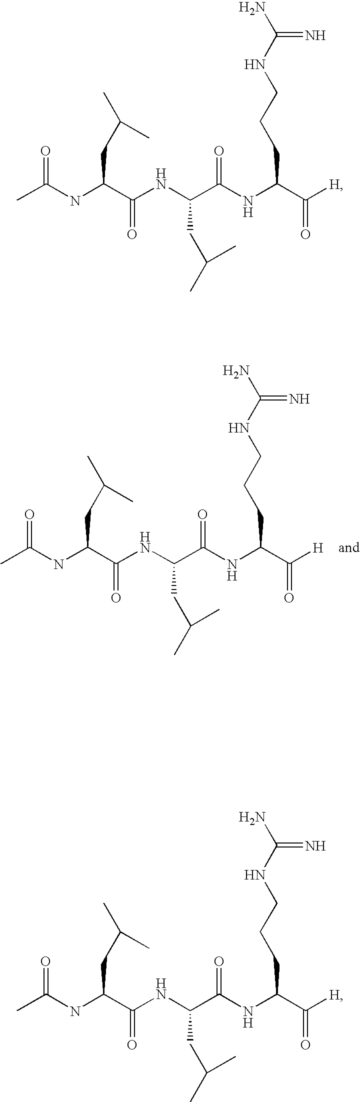
or a pharmaceutically acceptable salt, solvate or ester thereof.

7. The method of any of claims 1 to 5, wherein the compound is selected from the group consisting of: 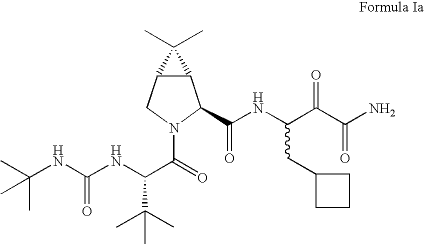
and pharmaceutically acceptable salts or solvates thereof.

8. The method of any of claims 1 to 5, wherein the compound is selected from the group consisting of: 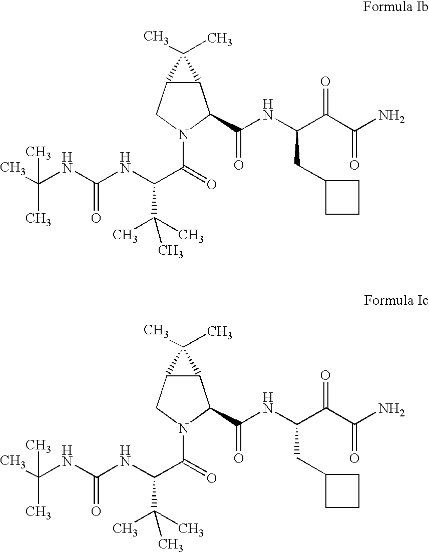
and pharmaceutically acceptable salts or solvates thereof.

9. The method of any of claims 1 to 5, wherein said dosage form comprises at least one dosage unit.

10. The method of claim 9, wherein said dosage form comprises a plurality of dosage units.

11. The method of claim 10, wherein said dosage form comprises from 2-100 dosage units.

12. The method of any of claims 1 to 5, wherein the dosage form is capable of maintaining an average Cmin plasma concentration of the at least one HCV protease inhibitor at or above about 50 ng/ml.

13. The method of any of claims 1 to 5, wherein the dosage form is capable of maintaining an average Cmin plasma concentration of the at least one HCV protease inhibitor at or above about 100 ng/ml.

14. The method of any of claims 1 to 5, wherein the dosage form is capable of maintaining an average Cmin plasma concentration of the at least one HCV protease inhibitor at or above about 150 ng/ml.

15. The method of any of claims 1 to 5, wherein the dosage form is capable of maintaining an average Cmin plasma concentration of the at least one HCV protease inhibitor at or above about 200 ng/ml.

16. The method of any of claims 1 to 5, wherein the dosage form is an oral dosage form.

17. The method of claim 16, wherein the oral dosage form is selected from the group consisting of tablets, capsules, caplets, solutions, suspensions, emulsions, troches, lozenges, effervescent tablets, lollipops, and reconstitutible powders.

18. The method of any of claims 1 to 5, wherein the dosage form is an injectable dosage form for intravenous or subcutaneous administration.

19. The method of any of claims 1 to 5, wherein the dosage form is an intranasal dosage form.

20. The method of any of claims 1 to 5, wherein the dosage form is an aerosol preparation.

21. The method of any of claims 1 to 5, wherein the dosage form is a suppository.

22. The method of any of claims 1 to 5, wherein the dosage form is a transdermal preparation.

23. The method of claim 22, wherein the transdermal preparation is selected from the group consisting of creams, lotions, aerosols, emulsions, and medicated patches.

24. The method of any of claims 1 to 5, wherein the dosage form contains from about 1 mg to about 3000 mg of the at least one compound.

25. The method of any of claims 1 or 5, wherein the dosage form is administered in an asymmetric pattern to coincide with a circadian rhythm of the subject.

26. The method of any of claims 1 to 5, wherein the at least one compound is administered as combination therapy with at least one of an antiviral agent which is different from the at least one compound and/or an immunomodulatory agent.

27. The method of claim 26, wherein the at least one of an antiviral agent which is different from the at least one compound and/or the immunomodulatory agent are administered concurrently or sequentially with the at least one compound.

28. The method claim 26, wherein the at least one of an antiviral agent which is different from the at least one compound and/or the immunomodulatory agent are a part of the dosage form.

29. The method of claim 26, wherein the at least one of an antiviral agent which is different from the at least one compound and/or the immunomodulatory agent are selected from the group consisting of ribavirin, levovirin, VP 50406, ISIS 14803, Heptazyme, VX 497, Thymosin, Maxamine, mycophenolate mofetil, interferon, and mixtures thereof.

30. The method of claim 29, wherein the interferon is selected from the group consisting of interferon-alpha, PEG-interferon alpha conjugates and consensus interferon.

31. The method of any of claims 1 to 5, further comprising at least one anti-cancer agent.
